(19) United States
(12) Patent Application Publication
(10) Pub. No.: US 2021/0391548 A1
(43) Pub. Date: Dec. 16, 2021

(54) ORGANIC LIGHT-EMITTING DEVICE

(71) Applicant: SAMSUNG ELECTRONICS CO., LTD., Suwon-si (KR)

(72) Inventors: Kyuyoung Hwang, Anyang-si (KR); Ohyun Kwon, Seoul (KR); Hyun Koo, Seoul (KR); Youngjae Park, Suwon-si (KR); Kum Hee Lee, Suwon-si (KR); Aram Jeon, Suwon-si (KR); Whail Choi, Seoul (KR); Byoungki Choi, Hwaseong-si (KR)

(21) Appl. No.: 17/381,374

(22) Filed: Jul. 21, 2021

Related U.S. Application Data
(63) Continuation of application No. 15/387,137, filed on Dec. 21, 2016.

(30) Foreign Application Priority Data
May 27, 2016 (KR) 10-2016-0065691

(51) Int. Cl. H01L 51/00; C09K 11/06; C09K 11/02; C07D 251/24; C07D 213/16; C07D 239/26; C07D 209/86; C07D 401/14

(52) U.S. Cl. CPC H01L 51/0094; H01L 51/5016; C09K 11/025; C07D 251/24; H01L 51/0054; H01L 51/0067; C07D 213/16; C07D 239/26; C07D 209/86; H01L 51/0072; C07D 401/14; H01L 51/0085; H01L 51/0084; C09K 2211/1007; C09K 2211/1029; C09K 2211/185; C09K 2211/1088; C09K 2211/1033; H01L 2251/5384; C09K 11/06

(57) ABSTRACT

An organic light-emitting device including a first electrode, a second electrode facing the first electrode, and an organic layer disposed between the first electrode and the second electrode, wherein the organic layer includes an emission layer, wherein the organic layer further includes i) an organometallic compound represented by Formula 1, and ii) at least one selected from a first compound represented by Formula 51, a second compound represented by Formula 61, a third compound represented by Formula 81, and a fourth compound represented by Formula 91, wherein Formulae 1, 51, 61, 81, and 91 are the same as described in the specification.

### CROSS-REFERENCE TO RELATED APPLICATION

This is a continuation application of U.S. application Ser. No. 15/387,137, filed on Dec. 21, 2016, which claims priority to Korean Patent Application No. 10-2016-0065691, filed on May 27, 2016, in the Korean Intellectual Property Office, the contents of which are incorporated herein in their entirety by reference.

### BACKGROUND

### 1. Field

One or more embodiments relate to an organic light-emitting device.

### 2. Description of the Related Art

Organic light-emitting devices (OLEDs) are self-emission devices that have wide viewing angles, high contrast ratios, and short response times. In addition, the OLEDs display excellent brightness, driving voltage, and response speed characteristics, and produce full-color images.

An example of such organic light-emitting devices includes an anode, a cathode, and an organic layer disposed between the anode and the cathode, wherein the organic layer includes an emission layer. A hole transport region may be disposed between the anode and the emission layer, and an electron transport region may be disposed between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. These excitons transition from an excited state to a ground state, thereby generating light.

Various types of organic light emitting devices are known. However, there still remains a need in OLEDs having low driving voltage, high efficiency, high brightness, and long lifespan.

### SUMMARY

One or more embodiments include a high-quality organic light-emitting device.

Additional aspects will be set forth in part in the description which follows and, in part, will be apparent from the description, or may be learned by practice of the presented embodiments.

According to one or more embodiments, an organic light-emitting device includes:

a first electrode;

a second electrode facing the first electrode; and

an emission layer disposed between the first electrode and the second electrode, the emission layer including an emission layer,

wherein the organic layer includes:

i) an organometallic compound represented by Formula 1; and

ii) at least one selected from a first compound represented by Formula 51, a second compound represented by Formula 61, a third compound represented by Formula 81, and a fourth compound represented by Formula 91:

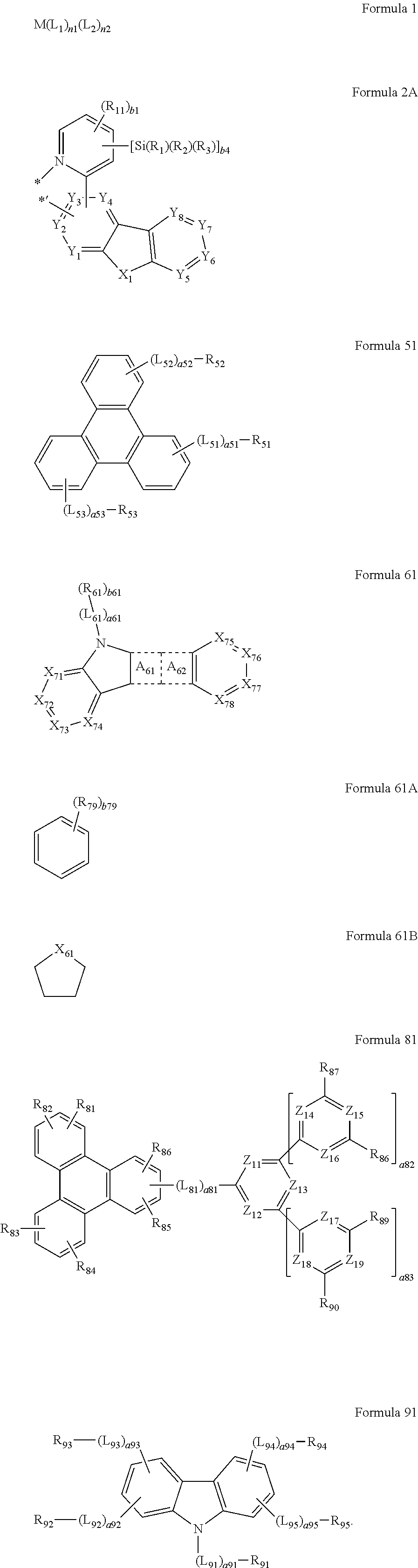

M in Formula 1 may be selected from iridium (Ir), platinum (Pt), osmium (Os), titanium (Ti), zirconium (Zr), hafnium (Hf), europium (Eu), terbium (Tb), thulium (Tm), and rhodium (Rh),

in Formula 1, L₁ may be a ligand represented by Formula 2A, and n1 may be 1, 2, or 3, wherein when n1 is two or more, two or more groups L₁ may be identical to or different from each other,

in Formula 1, L₂ may be selected from a monovalent organic ligand, a divalent organic ligand, a trivalent organic ligand, and a tetravalent organic ligand, and n2 may be 0, 1, 2, 3, or 4, wherein when n2 is two or more, two or more groups L₂ may be identical to or different from each other,

L₁ and L₂ in Formula 1 may be different from each other,

R₁ to R₃ in Formula 2A may each independently be selected from a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, and —Si(Q₅₁)(Q₅₂)(Q₅₃),

X₁ in Formula 2A may be selected from O, S, S(═O)₂, N(R₂₁), and Si(R₂₂)(R₂₃),

in Formula 2A, Y₁ may be N, C(R₄₁), or C linked to a pyridine ring, Y₂ may be N, C(R₄₂), or C linked to a pyridine ring, Y₃ may be N, C(R₄₃), or C linked to a pyridine ring, Y₄ may be N, C(R₄₄), or C linked to a pyridine ring, Y₅ may be N or C(R₄₅), Y₆ may be N or C(R₄₆), Y₇ may be N or C(R₄₇), Y₈ may be N or C(R₄₅), and one selected from Y₁ to Y₄ may be C linked to a pyridine ring,

R₁₁, R₂₁ to R₂₃, and R₄₁ to R₄₈ in Formula 2A may each independently be selected from hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), and —P(═O)(Q₈)(Q₉),

two or more groups R₁₁ may optionally be linked to form a saturated or unsaturated C₄-C₆₀ ring,

two or more selected from R₄₁ to R₄₄ may optionally be linked to form a saturated or unsaturated C₄-C₆₀ ring,

two or more selected from R₄₅ to R₄₈ may optionally be linked to form a saturated or unsaturated C₄-C₆₀ ring,

in Formula 2A, b1 may be an integer selected from 0 to 3, and b4 may be an integer selected from 1 to 4,

* and *′ in Formula 2A each indicate a binding site to M in Formula 1,

ring A₈₁ in Formula 61 may be represented by Formula 61A,

ring A62 in Formula 61 may be represented by Formula 61B,

X₆₁ in Formula 61B may be N-[(L₆₂)ₐ₆₂-(R₆₂)_{b62}], S, O, S(═O), S(═O)₂, C(═O), C(R₆₃)(R₆₄), Si(R₆₃)(R₆₄), P(R₆₃), P(═O)(R₆₃), or C═N(R₆₃),

in Formula 61, X₇₁ may be C(R₇₁) or N, X₇₂ may be C(R₇₂) or N, X₇₃ may be C(R₇₃) or N, X₇₄ may be C(R₇₄) or N, X₇₅ may be C(R₇₅) or N, X₇₆ may be C(R₇₆) or N, X₇₇ may be C(R₇₇) or N, and X₇₈ may be C(R₇₈) or N,

L₅₁ to L₅₃, L₈₁, L₆₂, and L₉₁ to L₉₅ in Formulae 51, 61, 61B, and 91 may each independently be selected from a single bond, a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,

a51 to a53, a61, a62, and a91 to a95 in Formulae 51, 61, 61B, and 91 may each independently be an integer selected from 1 to 5,

R₅₁ to R₅₃, R₈₁ to R₆₄, R₇₁ to R₇₉, and R₉₁ to R₉₅ in Formulae 51, 61, 61A, 61B, and 91 may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁₁)(Q₁₂), —Si(Q₁₃)(Q₁₄)(Q₁₅), and —B(Q₁₆)(Q₁₇),

b61, b62, and b79 in Formulae 61A and 61B may each independently be an integer selected from 0 to 3,

in Formula 81, Z₁₁ may be N or C(Rₐ), Z₁₂ may be N or C(R_{b}), Z₁₃ may be N or C(R_{c}), Z₁₄ may be N or C(R_{d}), Z₁₅ may be N or C(Rₑ), Z₁₀ may be N or C(R_{f}), Z₁₇ may be N or C(R_{g}), Z₁₈ may be N or C(Rₕ), Z₁₉ may be N or C(Rᵢ), and at least one selected from Z₁₁ to Z₁₉ may be N,

R₈₁ to R₉₀ and Rₐ to Rᵢ in Formula 81 may each independently be selected from hydrogen, deuterium, a substituted or unsubstituted C₁-C₁₀ alkyl group, and a substituted or unsubstituted C₆-C₁₂ aryl group,

the number of 6-membered rings substituted with a triphenylene core in Formula 81 may be six or less,

L₈₁ in Formula 81 may be selected from a substituted or unsubstituted phenylene group, a substituted or unsubstituted biphenylene group, and a substituted or unsubstituted terphenylene group,

a81 to a83 in Formula 81 may each independently be 0 or 1,

in Formula 81, a81+a82+a83≥1,

at least one selected from L₉₁ to L₉₅ and R₉₁ to R₉₅ in Formula 91 may include a carbazole ring or a triphenylene ring, and

at least one substituent selected from a substituent(s) of the substituted C₁-C₂₀ alkylene group, the substituted C₂-C₂₀ alkenylene group, the substituted C₃-C₁₀ cycloalkylene group, the substituted C₁-C₁₀ heterocycloalkylene group, the substituted C₃-C₁₀ cycloalkenylene group, the substituted C₁-C₁₀ heterocycloalkenylene group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, the substituted monovalent non-aromatic condensed heteropolycyclic group, the substituted phenylene group, the substituted biphenylene group, and the substituted terphenylene group may be selected from:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and

—N(Q₃₁)(Q₃₂), —Si(Q₃₃)(Q₃₄)(Q₃₅), and —B(Q₃₆)(Q₃₇),

wherein Q₁ to Q₉, Q₁₁ to Q₁₉, Q₂₁ to Q₂₉, to Q₃₉, and Q₅₁ to Q₅₃ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

### BRIEF DESCRIPTION OF THE DRAWINGS

These and/or other aspects will become apparent and more readily appreciated from the following description of the embodiments, taken in conjunction with the accompanying drawing in which:

FIG. 1 is a schematic cross-sectional view of an organic light-emitting device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present disclosure. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

An organic light-emitting device according to an embodiment may include:

a first electrode;

a second electrode facing the first electrode; and

an organic layer disposed between the first electrode and the second electrode,

wherein the organic layer includes an emission layer.

The organic layer may include:

i) an organometallic compound represented by Formula 1; and

ii) at least one selected from a first compound represented by Formula 51, a second compound represented by Formula 61, a third compound represented by Formula 81, and a fourth compound represented by Formula 91:

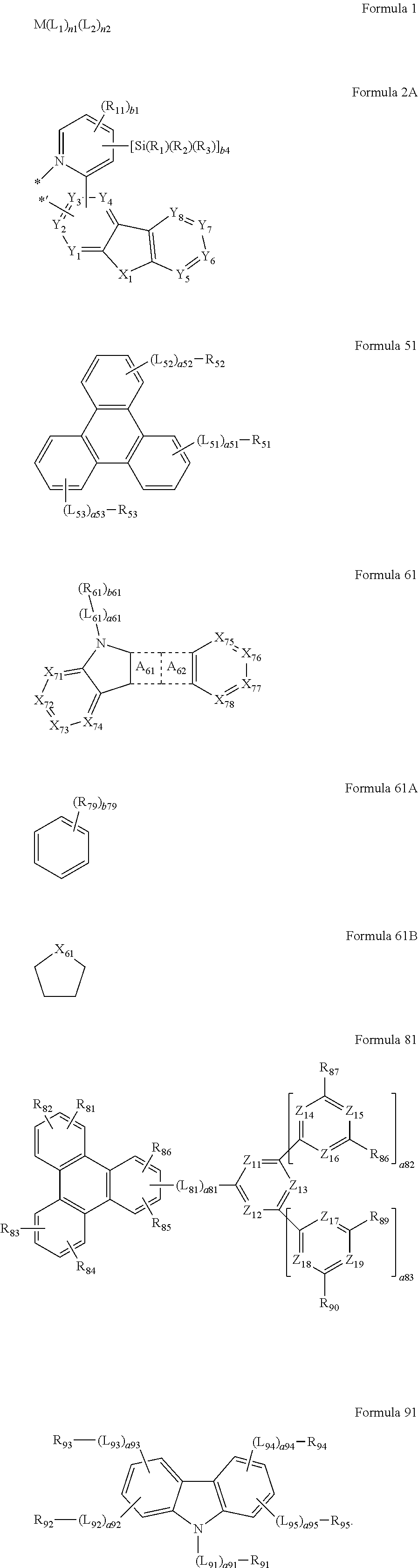

Hereinafter, the compounds will be sequentially described.

### Organometallic Compound Represented by Formula 1

L₂ in Formula 1 may be selected from a monovalent organic ligand, a divalent organic ligand, a trivalent organic ligand, and a tetravalent organic ligand, and n2 may be 0, 1, 2, 3, or 4, wherein when n2 is two or more, two or more groups L₂ may be identical to or different from each other.

L₁ and L₂ in Formula 1 may be different from each other.

In one or more embodiments, n1 in Formula 1 may be 1.

In one or more embodiments, the organometallic compound represented by Formula 1 may be an electrically neutral compound, not a salt consisting of an ionic pair.

In one or more embodiments, in Formula 1, M may be Ir and the sum of n1 and n2 may be 3; or M may be Pt and the sum of n1 and n2 may be 2, and the organometallic compound represented by Formula 1 may be electrically neutral.

R₁ to R₃ in Formula 2A may each independently be selected from a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, and —Si(Q₅₁)(Q₅₂)(Q₅₃).

In one or more embodiments, R₁ to R₃ in Formula 2A may each independently be selected from:

a C₁-C₂₀ alkyl group, a C₁-C₂₀ alkoxy group, and —Si(Q₅₁)(Q₅₂)(Q₅₃);

a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group; and

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group,

wherein Q₅₁ to Q₅₃ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium, a C₁-C₁₀ alkyl group, and a phenyl group,

but embodiments are not limited thereto.

In one or more embodiments, R₁ and R₃ in Formula 2A may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, a naphthyl group, and —Si(Q₅₁)(Q₅₂)(Q₅₃); and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium, a C₁-C₁₀ alkyl group, and a phenyl group,

wherein Q₅₁ to Q₅₃ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium, a C₁-C₁₀ alkyl group, and a phenyl group.

In one or more embodiments, Q₅₁ to Q₅₃ may each independently be a methyl group or an ethyl group.

In one or more embodiments, in Formula 2A,

R₁ to R₃ may all be identical to one another;

R₁ and R₃ may be identical to each other and R₂ and R₁ may be different from each other; or

R₁ to R₃ may all be different from one another.

X₁ in Formula 2A may be selected from O, S, S(═O)₂, N(R₂₁), and Si(R₂₂)(R₂₃).

In one or more embodiments, X₁ in Formula 2A may be selected from O, S, and N(R₂₁).

In one or more embodiments, X₁ in Formula 2A may be O, but is not limited thereto.

R₁₁, R₂₁ to R₂₃, and R₄₁ to R₄₈ in Formula 2A may each independently be selected from hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), and —P(═O)(Q₈)(Q₉).

In one or more embodiments, R₁₁, R₂₁ to R₂₃, and R₄₁ to R₄₈ in Formula 2A may each independently be selected from:

hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₁₄ aryl group, a C₁-C₁₄ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₁₄ aryl group, a C₁-C₁₄ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₁₄ aryl group, a C₁-C₁₄ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₁₄ aryl group, a C₁-C₁₄ heteroaryl group, a monovalent non-aromatic condensed polycyclic group, and a monovalent non-aromatic condensed heteropolycyclic group; and

—N(Q₁)(Q₂), —Si(Q₃)(Q₄)(Q₅), —B(Q₆)(Q₇), and —P(═O)(Q₈)(Q₉),

wherein Q₁ to Q₉ may each independently be selected from a substituted or unsubstituted C₁-C₂₀ alkyl group, a substituted or unsubstituted C₁-C₂₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₁₄ aryl group, a substituted or unsubstituted C₁-C₁₄ heteroaryl group, a substituted or unsubstituted monovalent non-aromatic condensed polycyclic group, and a substituted or unsubstituted monovalent non-aromatic condensed heteropolycyclic group.

In one or more embodiments, to R₂₃, and R₄₁ to R₄₈ in Formula 2A may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group; and

—B(Q₆)(Q₇) and —P(═O)(Q₈)(Q₉),

wherein Q₆ to Q₉ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium, a C₁-C₁₀ alkyl group, and a phenyl group,

but embodiments are not limited thereto.

In one or more embodiments, R₁₁, R₂₁ to R₂₃, and R₄₁ to R₄₈ in Formula 2A may each independently be selected from:

hydrogen, deuterium, —F, a cyano group, a nitro group, —SF₅, a methyl group, an ethyl group, an n-propyl group, an iso-propyl group, an n-butyl group, an iso-butyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group;

a methyl group, an ethyl group, an n-propyl group, an iso-propyl group, an n-butyl group, an iso-butyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group, each substituted with at least one selected from deuterium, —F, —CD₃, —CD₂H, —CDH₂, —CF₃, —CF₂H, —CFH₂, a cyano group, a nitro group, a C₁-C₁₀ alkyl group, a C₁-C₁₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group; and —B(Q₆)(Q₇) and —P(═O)(Q₈)(Q₉),

wherein Q₆ to Q₉ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium, a C₁-C₁₀ alkyl group, and a phenyl group.

In one or more embodiments, R₁₁, R₂₁ to R₂₃, and R₄₁ to R₄₈ in Formula 2A may each independently be selected from hydrogen, deuterium, —F, a cyano group, a nitro group, —SF₅, —CH₃, —CD₃, —CD₂H, —CDH₂, —CF₃, —CF₂H, —CFH₂, groups represented by Formulae 9-1 to 9-17, and groups represented by Formulae 10-1 to 10-30, but embodiments are not limited thereto:

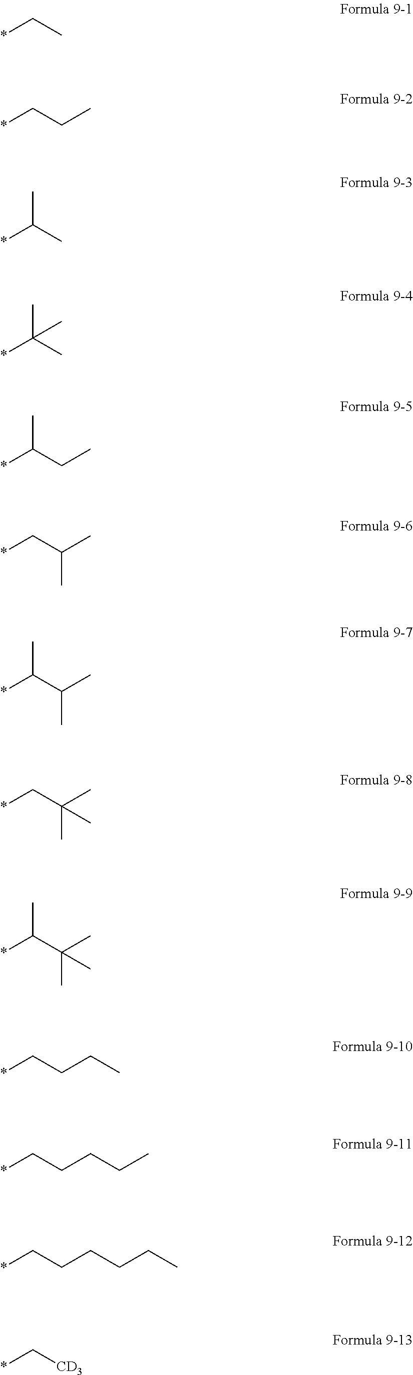

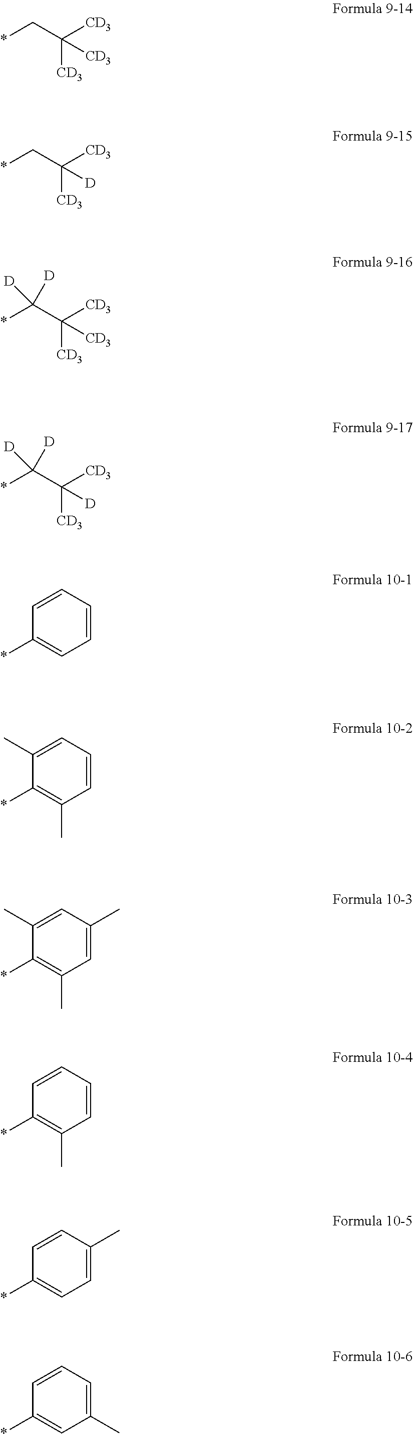

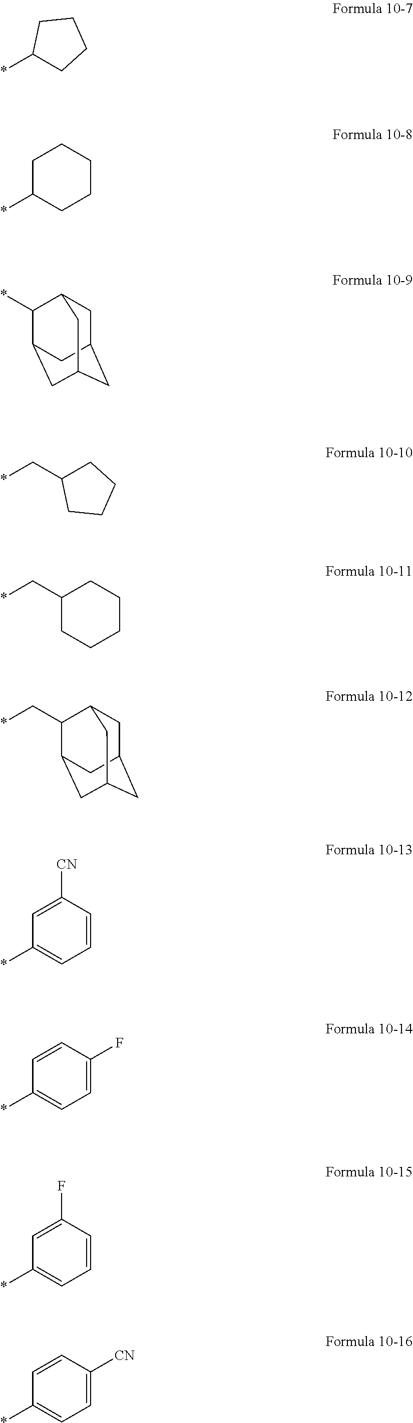

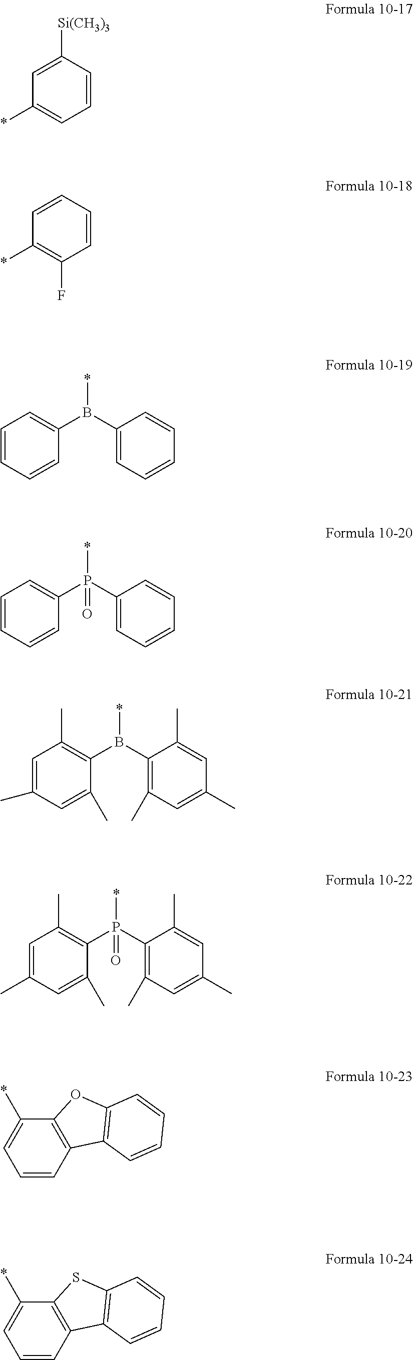

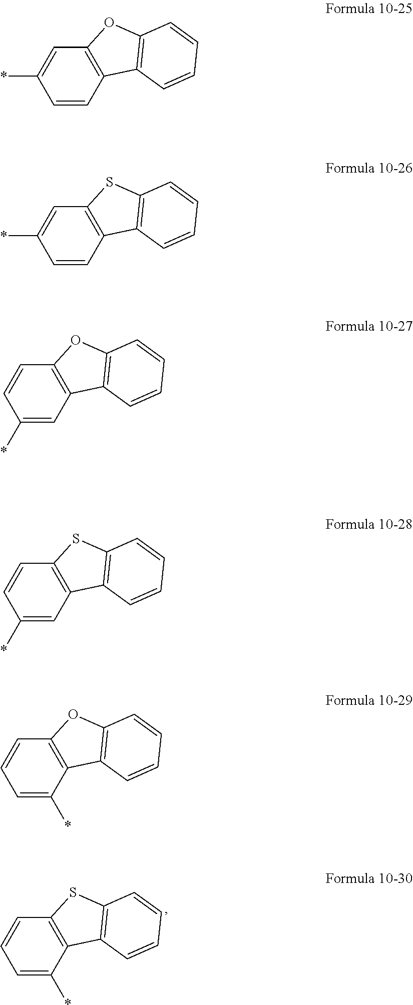 [SMILES: *c1ccc2c(c1)oc1ccccc12 ; *c1ccc2c(c1)sc1ccccc12 ; *c1ccc2oc3ccccc3c2c1 ; *c1ccc2sc3ccccc3c2c1 ; *c1cccc2oc3ccccc3c12 ; *c1cccc2sc3ccccc3c12]

wherein * in Formulae 9-1 to 9-17 and 10-1 to 10-30 indicates a binding site to a neighboring atom.

In Formula 2A, b1 may be an integer selected from 0 to 3, and b4 may be an integer selected from 1 to 4.

In one or more embodiments, R₂₁ to R₂₃ in X₁ of Formula 2A may each independently be selected from:

a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyridinyl group, a pyrimidinyl group, and a triazinyl group; and

a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyridinyl group, a pyrimidinyl group, and a triazinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, and a naphthyl group.

In Formula 2A, b1 indicates the number of groups R₁₁, wherein when b1 is two or more 2, two or more groups R₁₁ may be identical to or different from each other, and b4 indicates the number of groups —Si(R₁)(R₂)(R₃), wherein when b4 is two or more, two or more groups —Si(R₁)(R₂)(R₃) may be identical to or different from each other.

In one or more embodiments, in Formula 2A, b1 may be 0, 1, or 2, and b4 may be 1 or 2, but embodiments are not limited thereto.

In one or more embodiments, in Formula 2A, b1 may be 0, 1, or 2, and b4 may be 1, but embodiments of the present disclosure are not limited thereto.

in Formula 2A, two or more groups R₁₁ may optionally be linked to form a saturated or unsaturated C₄-C₆₀ ring (for example, a cyclopentane ring, a cyclohexane ring, an adamantane ring, a norbornane ring, a benzene ring, a pyridine ring, a pyrimidine ring, a naphthalene ring, a pyrene ring, a chrysene ring, or the like), two or more selected from R₄₁ to R₄₄ may optionally be linked to form a saturated or unsaturated C₄-C₆₀ ring (for example, a cyclopentane ring, a cyclohexane ring, an adamantane ring, a norbornane ring, a benzene ring, a pyridine ring, a pyrimidine ring, a naphthalene ring, a pyrene ring, a chrysene ring, or the like), and two or more selected from R₄₅ to R₄₈ may optionally be linked to form a saturated or unsaturated C₄-C₆₀ ring (for example, a cyclopentane ring, a cyclohexane ring, an adamantane ring, a norbornane ring, a benzene ring, a pyridine ring, a pyrimidine ring, a naphthalene ring, a pyrene ring, a chrysene ring, or the like).

In one or more embodiments, two groups R₁₁ in Formula 2A may be linked to form a substituted or unsubstituted cyclohexane or a substituted or unsubstituted benzene ring, but embodiments are not limited thereto.

In one or more embodiments, Y₁ to Y₈ in Formula 2A may not all be N.

In one or more embodiments, Y₁ or Y₃ in Formula 2A may be N.

In one or more embodiments, Y₅ or Y₆ in Formula 2A may be N.

In one or more embodiments, one or two selected from Y₃, Y₅, and Y₆ in Formula 2A may be N.

In one or more embodiments, L₁ in Formula 1 may be selected from ligands represented by Formulae 2A-1 to 2A-16:

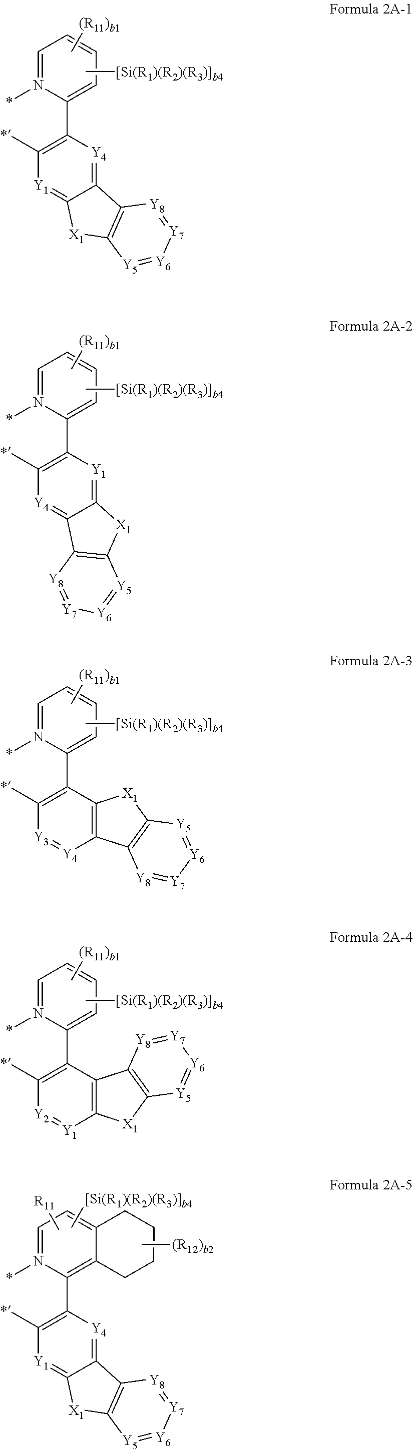

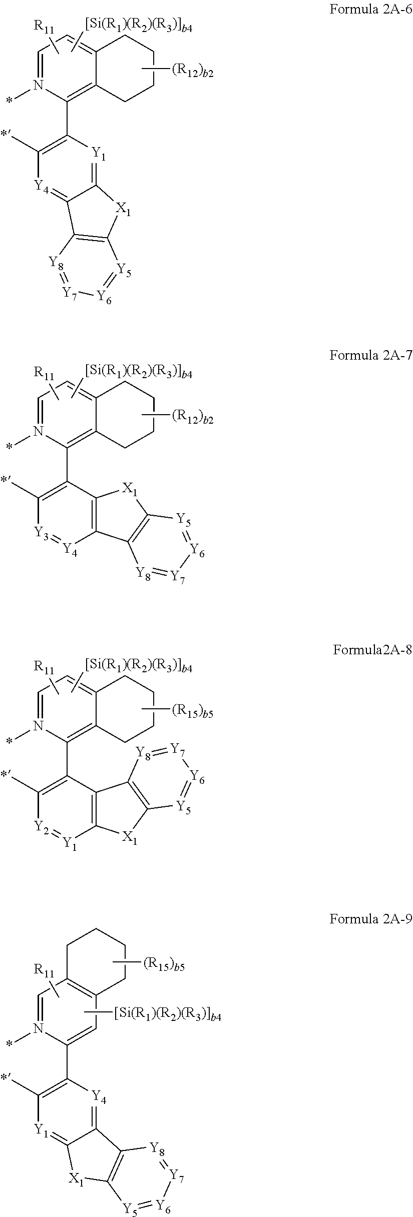

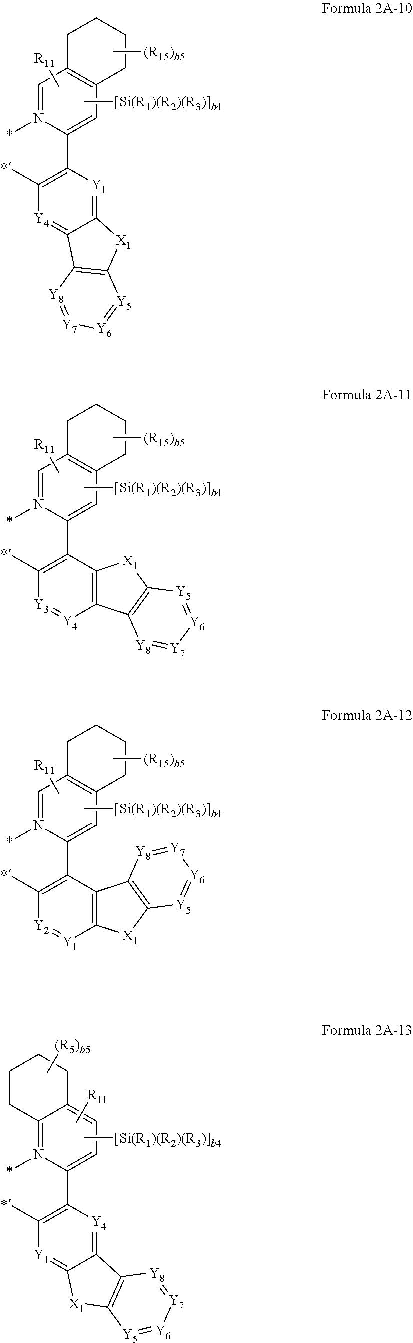

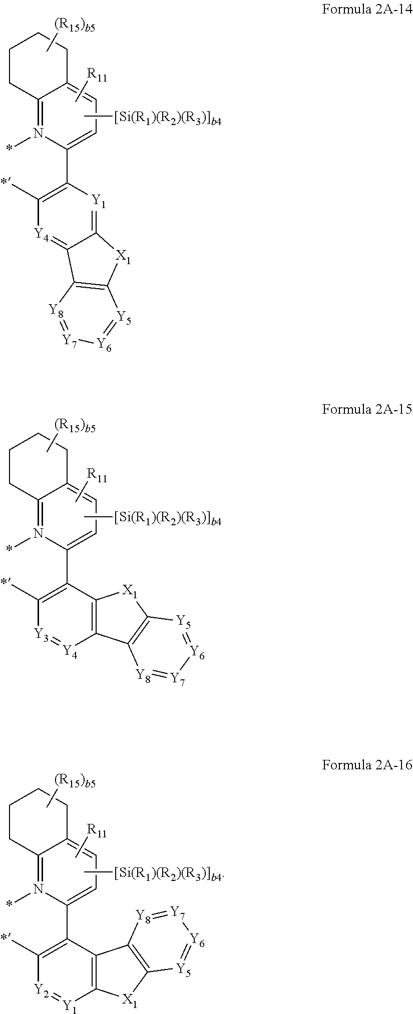

In Formulae 2A-1 to 2A-16, R₁ to R₃, Y₁ to Y₈, R₁₁, b1, and b4 are the same as described above, R₁₅ is the same as described above in connection with R₁₁, b5 may be an integer selected from 0 to 8, and * and *′ each indicate a binding site to M in Formula 1.

In one or more embodiments,

in Formulae 2A-1 to 2A-16, Y₁ may be C(R₄₁), Y₂ may be C(R₄₁), Y₃ may be C(R₄₃), Y₄ may be C(R₄₄), Y₅ may be C(R₄₅), Y₆ may be C(R₄₅), Y₇ may be C(R₄₇), and Y₈ may be C(R₄₅);

in Formulae 2A-1, 2A-2, 2A-4, 2A-5, 2A-6, 2A-8, 2A-9, 2A-10, 2A-12, 2A-13, 2A-14, and 2A-16, Y₁ may be N, Y₄ may be C(R₄₄), Y₅ may be C(R₄₅), Y₆ may be C(R₄₆), Y₇ may be C(R₄₇), and Y₈ may be C(R₄₅);

in Formulae 2A-1, 2A-2, 2A-4, 2A-5, 2A-6, 2A-8, 2A-9, 2A-10, 2A-12, 2A-13, 2A-14, and 2A-16, Y₁ may be C(R₄₁), Y₄ may be C(R₄₄), Y₅ may be N, Y₆ may be C(R₄₆), Y₇ may be C(R₄₇), and Y₈ may be C(R₄₅);

in Formulae 2A-1, 2A-2, 2A-4, 2A-5, 2A-6, 2A-8, 2A-9, 2A-10, 2A-12, 2A-13, 2A-14, and 2A-16, Y₁ may be C(R₄₁), Y₄ may be C(R₄₄), Y₅ may be C(R₄₅), Y₆ may be N, Y₇ may be C(R₄₇), and Y₈ may be C(R₄₅);

in Formulae 2A-1, 2A-2, 2A-4, 2A-5, 2A-6, 2A-8, 2A-9, 2A-10, 2A-12, 2A-13, 2A-14, and 2A-16, Y₁ may be N, Y₄ may be C(R₄₄), Y₅ may be N, Y₆ may be C(R₄₆), Y₇ may be C(R₄₇), and Y₈ may be C(R₄₅);

in Formulae 2A-1, 2A-2, 2A-4, 2A-5, 2A-6, 2A-8, 2A-9, 2A-10, 2A-12, 2A-13, 2A-14, and 2A-16, Y₁ may be N, Y₄ may be C(R₄₄), Y₅ may be C(R₄₅), Y₆ may be N, Y₇ may be C(R₄₇), and Y₈ may be C(R₄₅);

in Formulae 2A-3, 2A-7, 2A-11, and 2A-15, Y₃ may be N, Y₄ may be C(R₄₄), Y₅ may be C(R₄₅), Y₆ may be C(R₄₆), Y₇ may be C(R₄₇), and Y₈ may be C(R₄₅);

in Formulae 2A-3, 2A-7, 2A-11, and 2A-15, Y₃ may be C(R₄₃), Y₄ may be C(R₄₄), Y₅ may be N, Y₆ may be C(R₄₆), Y₇ may be C(R₄₇), and Y₈ may be C(R₄₅);

in Formulae 2A-3, 2A-7, 2A-11, and 2A-15, Y₃ may be C(R₄₃), Y₄ may be C(R₄₄), Y₅ may be C(R₄₅), Y₆ may be N, Y₇ may be C(R₄₇), and Y₈ may be C(R₄₅);

in Formulae 2A-3, 2A-7, 2A-11, and 2A-15, Y₃ may be N, Y₄ may be C(R₄₄), Y₅ may be N, Y₆ may be C(R₄₆), Y₇ may be C(R₄₇), and Y₈ may be C(R₄₅); or

in Formulae 2A-3, 2A-7, 2A-11, and 2A-15, Y₃ may be N, Y₄ may be C(R₄₄), Y₅ may be C(R₄₅), Y₆ may be N, Y₇ may be C(R₄₇), and Y₈ may be C(R₄₅), but embodiments are not limited thereto.

A maximum emission wavelength may be reduced in the following order: i) an organometallic compound including a ligand represented by Formula 2A-4, 2A-8, 2A-12, or 2A-16, ii) an organometallic compound including a ligand represented by Formula 2A-2, 2A-6, 2A-10, or 2A-14, iii) an organometallic compound including a ligand represented by Formula 2A-3, 2A-7, 2A-11, or 2A-15, and iv) an organometallic compound including a ligand represented by Formula 2A-1, 2A-5, 2A-9, or 2A-13. That is, the maximum emission wavelength of the organometallic compound including the ligand represented by Formula 2A-1, 2A-5, 2A-9, or 2A-13 in Formula 2A-1 to 2A-16 is lowest.

In one or more embodiments, L₁ in Formula 1 may be selected from ligands represented by Formulae 2AA-1, 2AA-2, 2AA-3, 2AA-4, and 2AB. For example, when L₁ in Formula 1 is selected from ligands represented by Formulae 2AA-1 and 2AB, the organic light-emitting device including the organometallic compound represented by Formula 1 may have high efficiency and long lifespan characteristics:

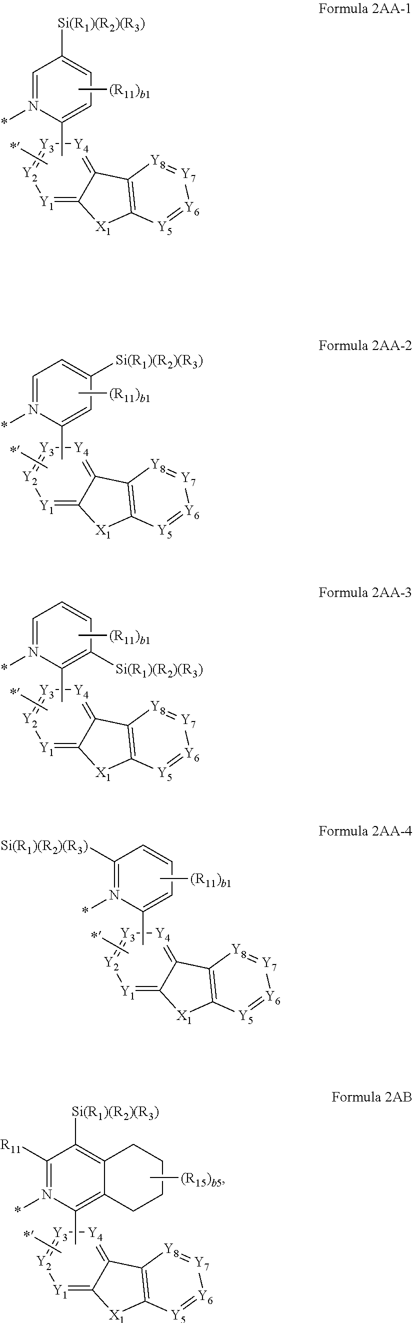

wherein, in Formulae 2AA-1, 2AA-2, 2AA-3, 2AA-4, and 2AB, R₁ to R₃, Y₁ to Y₈, R₁₁, and b1 are the same as described above, R₁₅ is the same as described above in connection with R₁₁, b5 may be an integer selected from 0 to 8, and * and *′ each indicate a binding site to M in Formula 1.

In one or more embodiments, in Formula 2A-1 to 2A-16, 2AA-1, 2AA-2, 2AA-3, 2AA-4, and 2AB,

R₁ to R₃ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, a naphthyl group, and —Si(Q₅₁)(Q₅₂)(Q₅₃); and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium, a C₁-C₁₀ alkyl group, and a phenyl group,

wherein Q₅₁ to Q₅₃ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium, a C₁-C₁₀ alkyl group, and a phenyl group,

X₁ may be selected from O, S, S(═O)₂, N(R₂₁), and Si(R₂₂)(R₂₃),

Y₁ to Y₈ are the same as described above,

R₁₁, R₁₅, R₂₁ to R₂₃, and R₄₁ to R₄₈ may each independently be selected from:

hydrogen, deuterium, —F, a cyano group, a nitro group, —SF₅, a methyl group, an ethyl group, an n-propyl group, an iso-propyl group, an n-butyl group, an iso-butyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group;

a methyl group, an ethyl group, an n-propyl group, an iso-propyl group, an n-butyl group, an iso-butyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group, each substituted with at least one selected from deuterium, —F, —CD₃, —CD₂H, —CDH₂, —CF₃, —CF₂H, —CFH₂, a cyano group, a nitro group, a C₁-C₁₀ alkyl group, a C₁-C₁₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group; and

—B(Q₆)(Q₇) and —P(═O)(Q₈)(Q₉),

wherein Q₆ to Q₉ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium and a C₁-C₁₀ alkyl group,

b1 may be an integer selected from 0 to 3,

b4 may be 1 or 2, and

b5 may be an integer selected from 0 to 8, but embodiments are not limited thereto.

In one or more embodiments, L₁ in Formula 1 may be selected from ligands represented by Formulae 2A(1) and 2A(40):

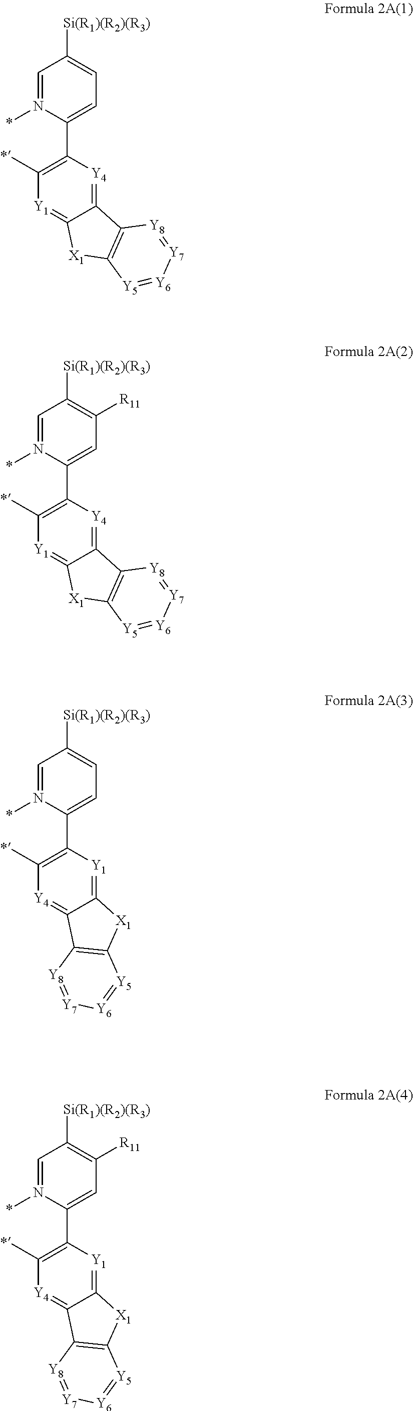

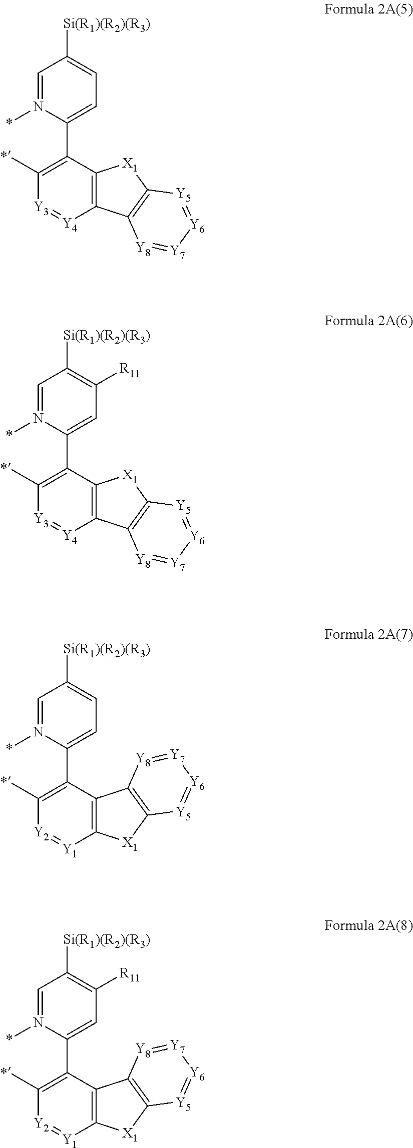

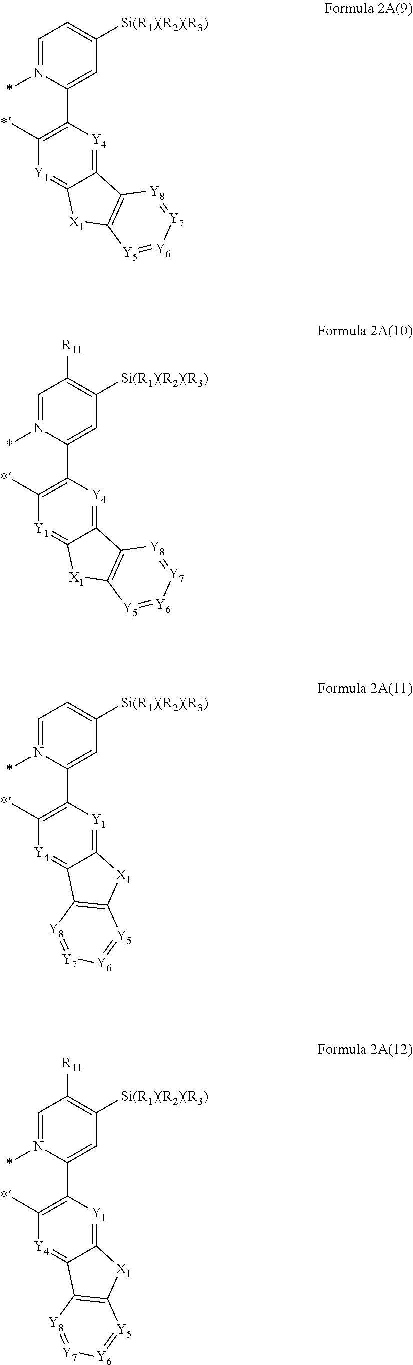

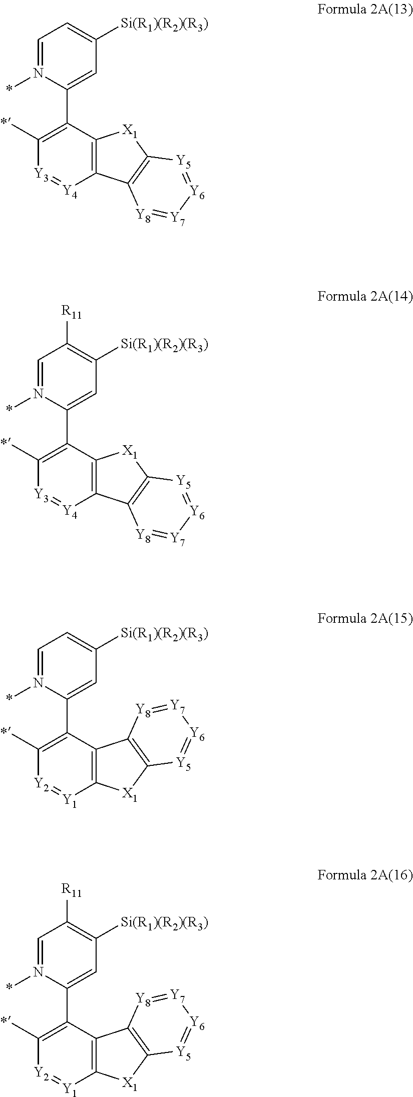

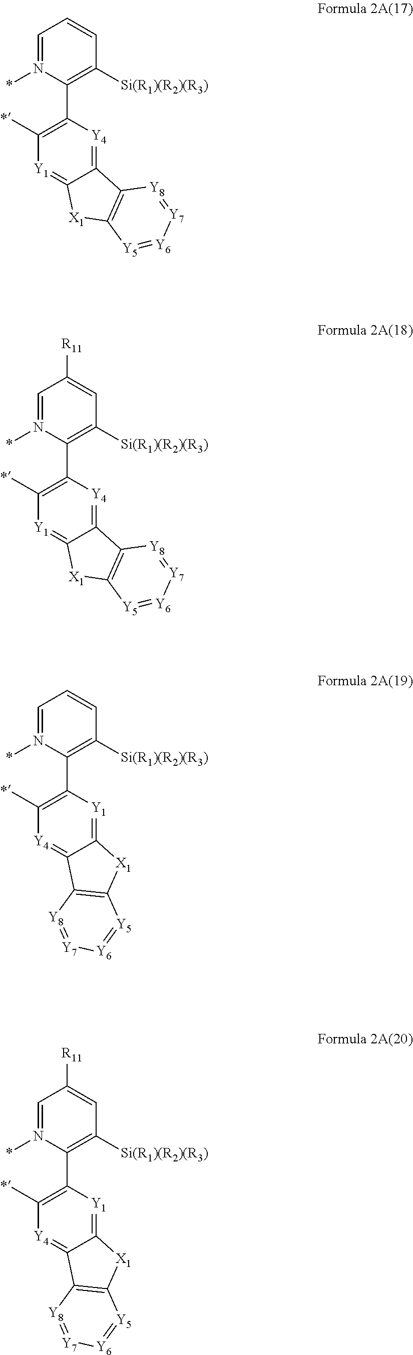

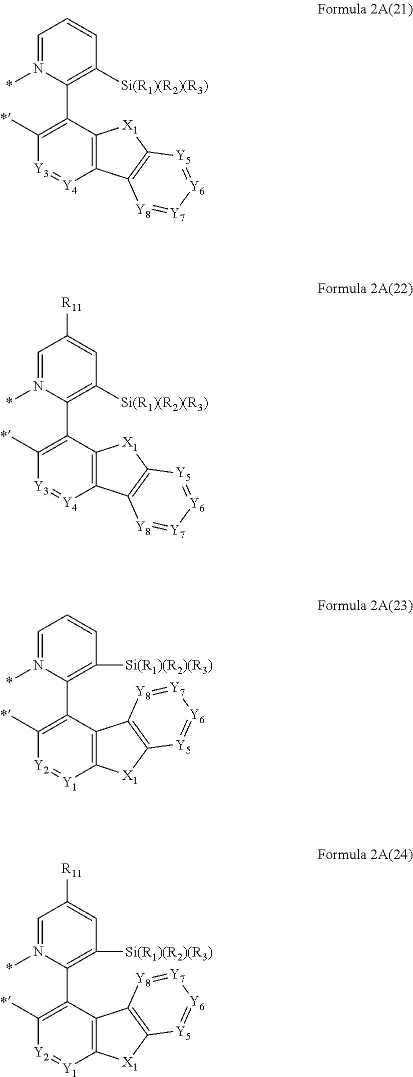

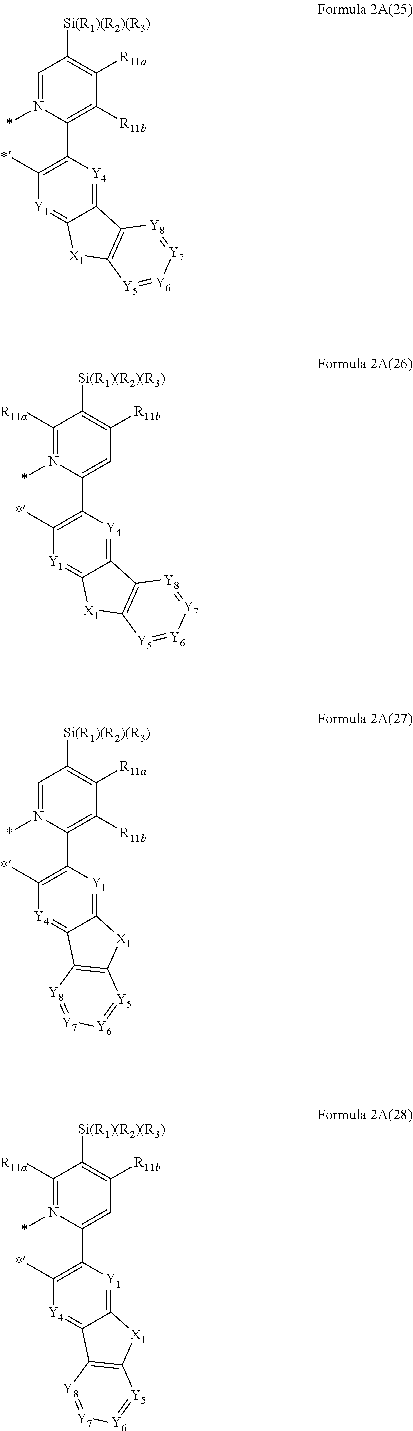

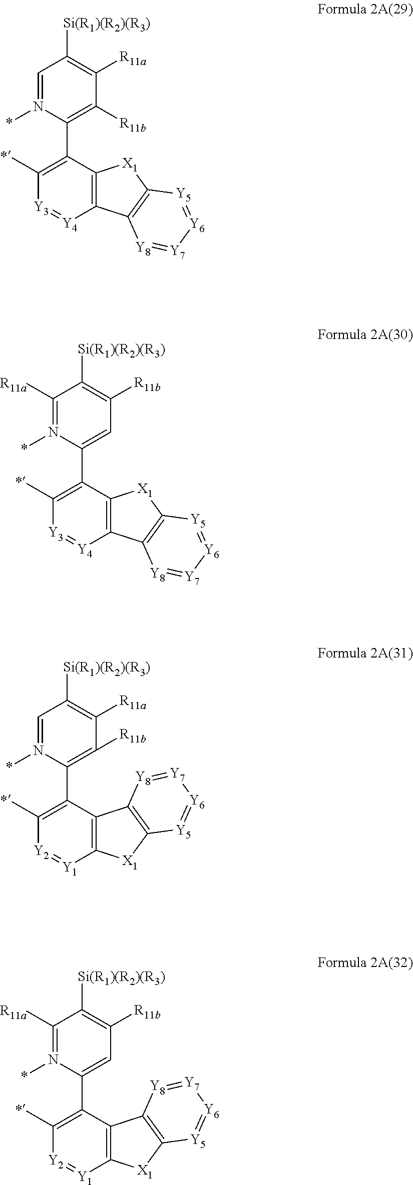

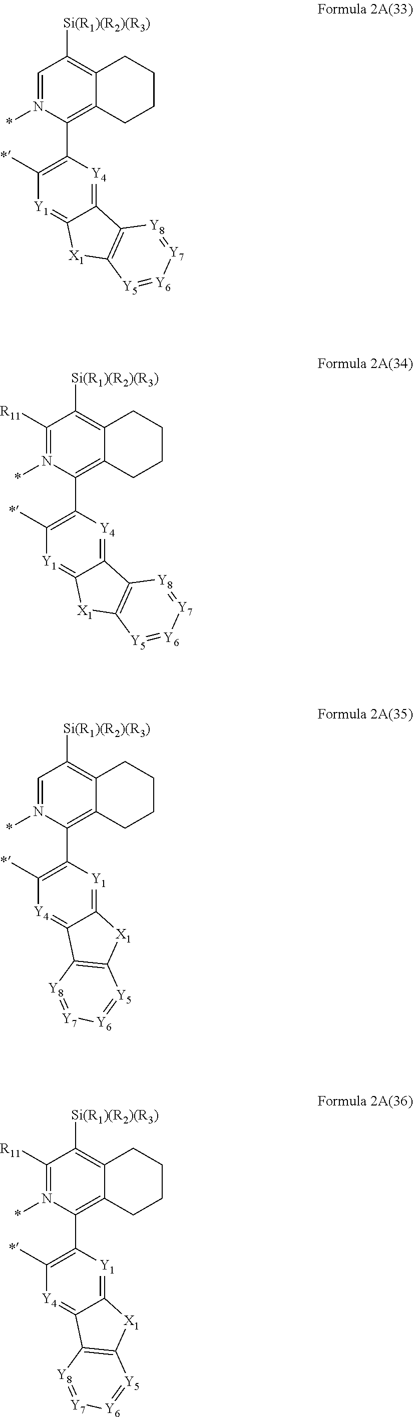

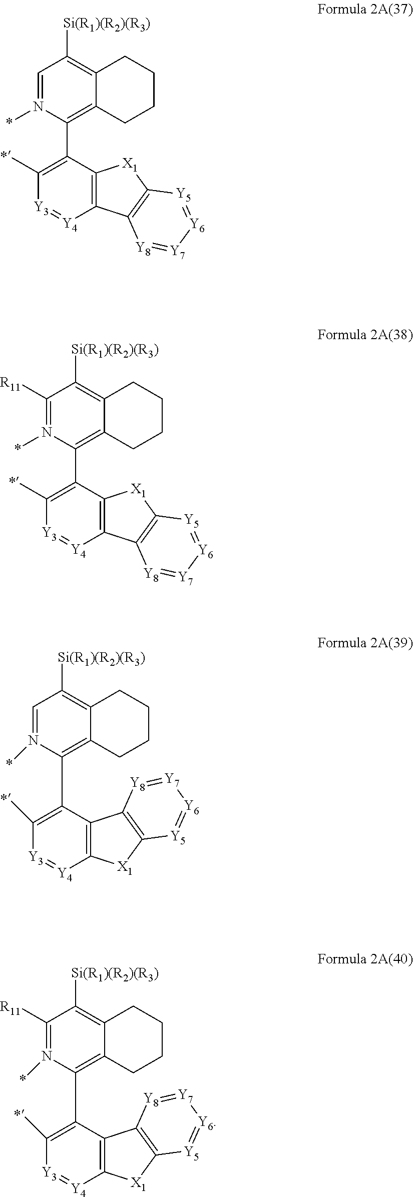

In Formulae 2A(1) to 2A(40), R₁ to R₃, Y₁ to Y₈, and R₁₁ are the same as described above, R₁₁ₐ and R_{11b} are the same as described above in connection with R₁₁, and * and *′ each indicate a binding site to M in Formula 1, wherein R₁₁, R₁₁ₐ, and R_{11b} may not be hydrogen.

In one or more embodiments,

in Formulae 2A(1) to 2A(40), Y₁ may be C(R₄₁), Y₂ may be C(R₄₁), Y₃ may be C(R₄₃), Y₄ may be C(R₄₄), Y₅ may be C(R₄₅), Y₆ may be C(R₄₈), Y₇ may be C(R₄₇), and Y₈ may be C(R₄₅);

in Formulae 2A(1) to 2(4), 2A(7) to 2A(12), 2A(15) to 2A(20), 2A(23) to 2A(28), and 2A(31) to 2A(36), Y₁ may be N, Y₄ may be C(R₄₄), Y₅ may be C(R₄₅), Y₆ may be C(R₄₆), Y₇ may be C(R₄₇), and Y₈ may be C(R₄₅);

in Formulae 2A(1) to 2(4), 2A(7) to 2A(12), 2A(15) to 2A(20), 2A(23) to 2A(28), and 2A(31) to 2A(36), Y₁ may be C(R₄₁), Y₄ may be C(R₄₄), Y₅ may be N, Y₆ may be C(R₄₆), Y₇ may be C(R₄₇), and Y₈ may be C(R₄₈);

in Formulae 2A(1) to 2(4), 2A(7) to 2A(12), 2A(15) to 2A(20), 2A(23) to 2A(28), and 2A(31) to 2A(36), Y₁ may be C(R₄₁), Y₄ may be C(R₄₄), Y₅ may be C(R₄₅), Y₆ may be N, Y₇ may be C(R₄₇), and Y₈ may be C(R₄₈);

in Formulae 2A(1) to 2(4), 2A(7) to 2A(12), 2A(15) to 2A(20), 2A(23) to 2A(28), and 2A(31) to 2A(36), Y₁ may be N, Y₄ may be C(R₄₄), Y₅ may be N, Y₆ may be C(R₄₆), Y₇ may be C(R₄₇), and Y₈ may be C(R₄₈);

in Formulae 2A(1) to 2(4), 2A(7) to 2A(12), 2A(15) to 2A(20), 2A(23) to 2A(28), and 2A(31) to 2A(36), Y₁ may be N, Y₄ may be C(R₄₄), Y₅ may be C(R₄₅), Y₆ may be N, Y₇ may be C(R₄₇), and Y₈ may be C(R₄₈);

in Formulae 2A(5), 2A(6), 2A(13), 2A(14), 2A(21), 2A(22), 2A(29), 2A(30), 2A(37), and 2A(38), Y₃ may be N, Y₄ may be C(R₄₄), Y₅ may be C(R₄₅), Y₆ may be C(R₄₈), Y₇ may be C(R₄₇), and Y₈ may be C(R₄₈);

in Formulae 2A(5), 2A(6), 2A(13), 2A(14), 2A(21), 2A(22), 2A(29), 2A(30), 2A(37), and 2A(38), Y₃ may be C(R₄₃), Y₄ may be C(R₄₄), Y₅ may be N, Y₆ may be C(R₄₈), Y₇ may be C(R₄₇), and Y₈ may be C(R₄₈);

in Formulae 2A(5), 2A(6), 2A(13), 2A(14), 2A(21), 2A(22), 2A(29), 2A(30), 2A(37), and 2A(38), Y₃ may be C(R₄₃), Y₄ may be C(R₄₄), Y₅ may be C(R₄₅), Y₆ may be N, Y₇ may be C(R₄₇), and Y₈ may be C(R₄₈);

in Formulae 2A(5), 2A(6), 2A(13), 2A(14), 2A(21), 2A(22), 2A(29), 2A(30), 2A(37), and 2A(38), Y₃ may be N, Y₄ may be C(R₄₄), Y₅ may be N, Y₆ may be C(R₄₆), Y₇ may be C(R₄₇), and Y₈ may be C(R₄₈); or

in Formulae 2A(5), 2A(6), 2A(13), 2A(14), 2A(21), 2A(22), 2A(29), 2A(30), 2A(37), and 2A(38), Y₃ may be N, Y₄ may be C(R₄₄), Y₅ may be C(R₄₅), Y₆ may be N, Y₇ may be C(R₄₇), and Y₈ may be C(R₄₈), but embodiments are not limited thereto.

In one or more embodiments, in Formulae 2A(1) and 2A(40),

R₁ to R₃ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, a naphthyl group, and —Si(Q₅₁)(Q₅₂)(Q₅₃); and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium, a C₁-C₁₀ alkyl group, and a phenyl group,

wherein Q₅₁ to Q₅₃ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium, a C₁-C₁₀ alkyl group, and a phenyl group,

X₁ may be selected from O, S, S(═O)₂, N(R₂₁), and Si(R₂₂)(R₂₃),

Y₁ to Y₈ are the same as described above, and

R₁₁, R₁₁ₐ, R_{11b}, R₂₁ to R₂₃, and R₄₁ to R₄₈ may each independently be selected from:

hydrogen, deuterium, —F, a cyano group, a nitro group, —SF₅, a methyl group, an ethyl group, an n-propyl group, an iso-propyl group, an n-butyl group, an iso-butyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group;

a methyl group, an ethyl group, an n-propyl group, an iso-propyl group, an n-butyl group, an iso-butyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group, each substituted with at least one selected from deuterium, —F, —CD₃, —CD₂H, —CDH₂, —CF₃, —CF₂H, —CFH₂, a cyano group, a nitro group, a C₁-C₁₀ alkyl group, a C₁-C₁₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group; and

—B(Q₆)(Q₇) and —P(═O)(Q₈)(Q₉),

(provided that R₁₁, R₁₁ₐ, and R_{11b} in Formulae 2A(1) to 2A(40) are not hydrogen),

wherein Q₆ to Q₉ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium and a C₁-C₁₀ alkyl group, but embodiments are not limited thereto.

In one or more embodiments, in Formulae 2A(1) to 2A(40),

R₁ to R₃ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, a naphthyl group, and —Si(Q₅₁)(Q₅₂)(Q₅₃); and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium, a C₁-C₁₀ alkyl group, and a phenyl group,

wherein Q₅₁ to Q₅₃ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium, a C₁-C₁₀ alkyl group, and a phenyl group,

X₁ may be O, S, S(═O)₂, N(R₂₁), and Si(R₂₂)(R₂₃),

Y₁ to Y₈ are the same described above,

R₁₁, R₁₁ₐ, R_{11b}, and R₄₁ to R₄₈ may be selected from deuterium, —F, a cyano group, a nitro group, —SF₅, —CH₃, —CD₃, —CD₂H, —CDH₂, —CF₃, —CF₂H, —CFH₂, groups represented by Formulae 9-1 to 9-17, and groups represented by Formulae 10-1 to 10-30,

R₂₁ to R₂₃ may each independently be selected from:

a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyridinyl group, a pyrimidinyl group, and a triazinyl group; and

a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyridinyl group, a pyrimidinyl group, and a triazinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, and a naphthyl group,

but embodiments are not limited thereto.

L₂ in Formula 1 may be selected from ligands represented by Formulae 3A to 3G:

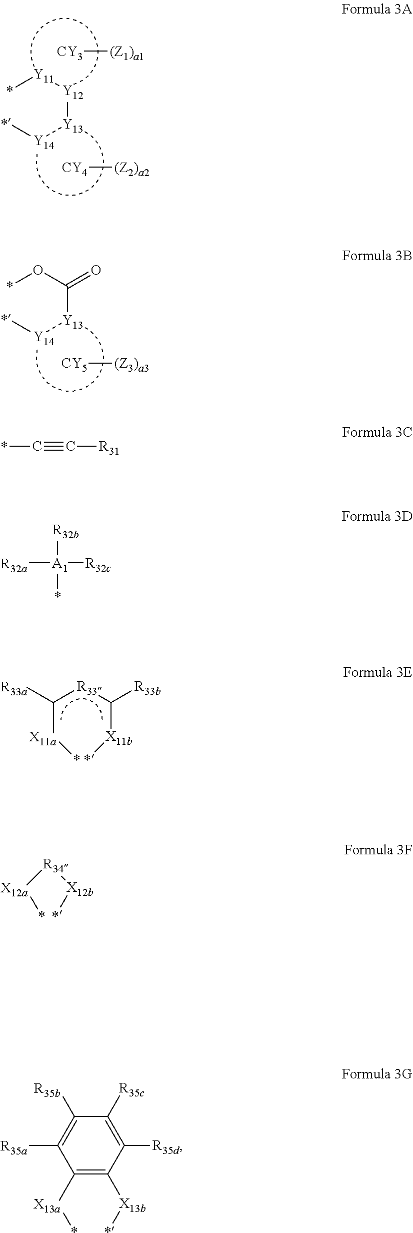

wherein, in Formulae 3A to 3G,

Y₁₁ to Y₁₀ may each independently be carbon (C) or nitrogen (N), Y₁₁ and Y₁₂ may be linked via a single bond or a double bond, Y₁₃ and Y₁₄ may be linked via a single bond or a double bond, and Y₁₅ and Y₁₀ may be linked via a single bond or a double bond,

CY₃ to CY₅ may each independently be selected from a C₅-C₆₀ cyclic group and a C₂-C₆₀ heterocyclic group (which may be a monocyclic group or a polycyclic group), and CY₃ and CY₄ may optionally be additionally linked via an organic linking group,

a1 to a3 may each independently be an integer selected from 1 to 5, A₁ may be P or As,

X₁₁ₐ, X_{11b}, X₁₂ₐ, X_{12b}, X₁₃ₐ, and X_{13b} may each independently be selected from N, O, N(R₃₄), P(R₃₅)(R₃₆), and As(R₃₇)(R₃₈) (provided that X₁₂ₐ, X_{12b}, X₁₃ₐ, and X_{13b} are neither N nor O);

R_{33″} and R_{34″} may each independently be selected from a single bond, a double bond, a substituted or unsubstituted C₁-C₅ alkylene group, a substituted or unsubstituted C₂-C₅ alkenylene group, and a substituted or unsubstituted C₆-C₁₀ arylene group;

Z₁ to Z₃, R₃₁, R₃₂ₐ, R_{32b}, R_{32c}, R₃₃ₐ, R_{33b}, R₃₄ to R₃₈, R₃₅ₐ, R_{35b}, R_{35c}, and R_{35d} may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), and —P(═O)(Q₈)(Q₉),

* and *′ each indicate a binding site to M in Formula 1, and

at least one substituent selected from a substituent(s) of the substituted C₁-C₅ alkylene group, the substituted C₂-C₅ alkenylene group, the substituted C₆-C₁₀ arylene group, the substituted C₁-C₆₀ alkyl group, the substituted C₂-C₈₀ alkenyl group, the substituted C₂-C₈₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and

—N(Q₃₁)(Q₃₂), —Si(Q₃₃)(Q₃₄)(Q₃₅), —B(Q₃₆)(Q₃₇), and —P(═O)(Q₃₈)(Q₃₉),

wherein Q₁ to Q₉, Q₁₁ to Q₁₉, to Q₂₉, and to Q₃₉ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

In one or more embodiments, CY₃ to CY₅ in Formulae 3A and 3B may each independently be selected from a benzene group, a naphthalene group, a fluorene group, a spiro-bifluorene group, an indene group, a pyrrole group, a thiophene group, a furan group, an imidazole group, a pyrazole group, a thiazole group, an isothiazole group, an oxazole group, an isoxazole group, a pyridine group, a pyrazine group, a pyrimidine group, a pyridazine group, a quinoline group, an isoquinoline group, a benzoquinoline group, a quinoxaline group, a quinazoline group, a carbazole group, a benzimidazole group, a benzofuran group, a benzothiophene group, an isobenzothiophene group, a benzoxazole group, an isobenzoxazole group, a triazole group, a tetrazole group, an oxadiazole group, a triazine group, a dibenzofuran group, a dibenzothiophene group, and a 5,6,7,8-tetrahydroisoquinoline group, but embodiments are not limited thereto.

In one or more embodiments, L₂ in Formula 1 may be selected from ligands represented by Formulae 3-1 to 3-111, but are not limited thereto:

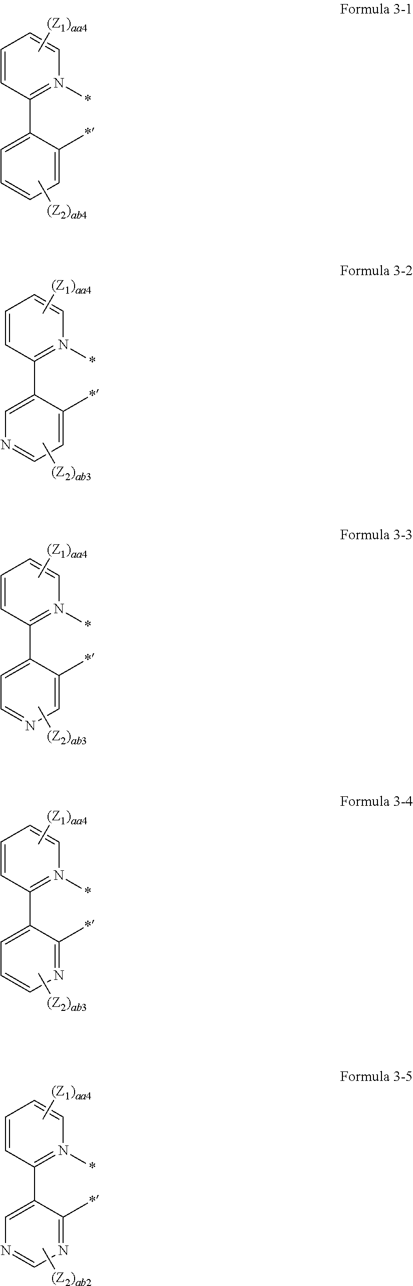

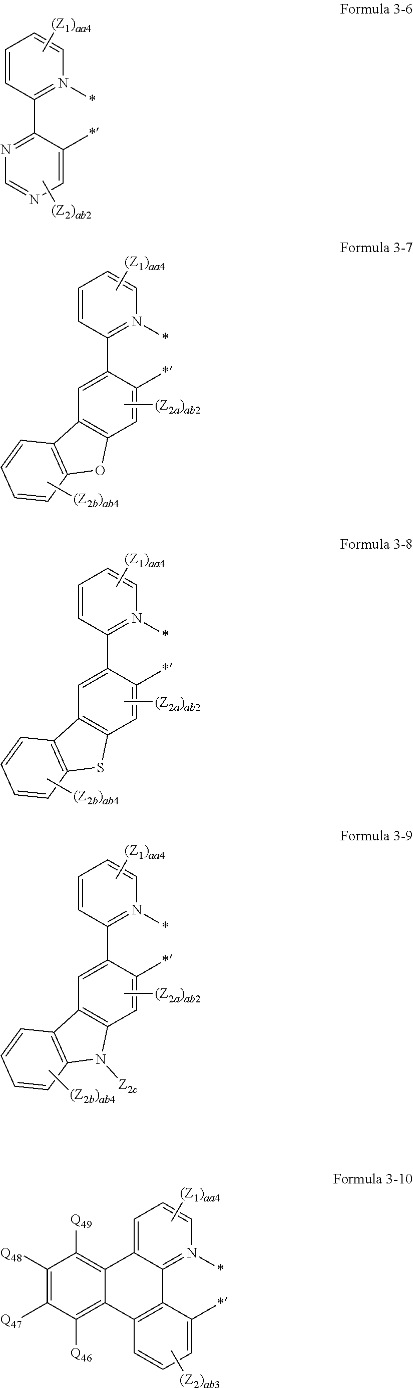

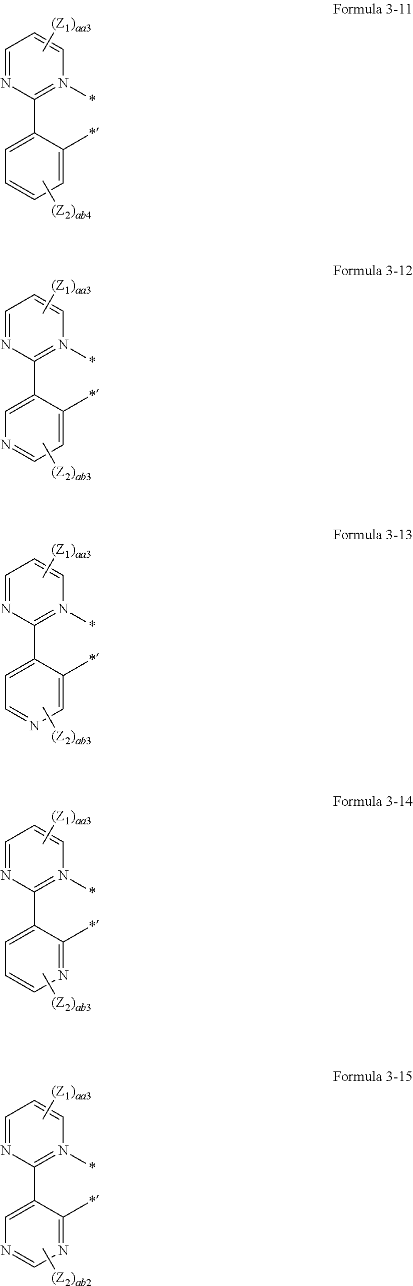

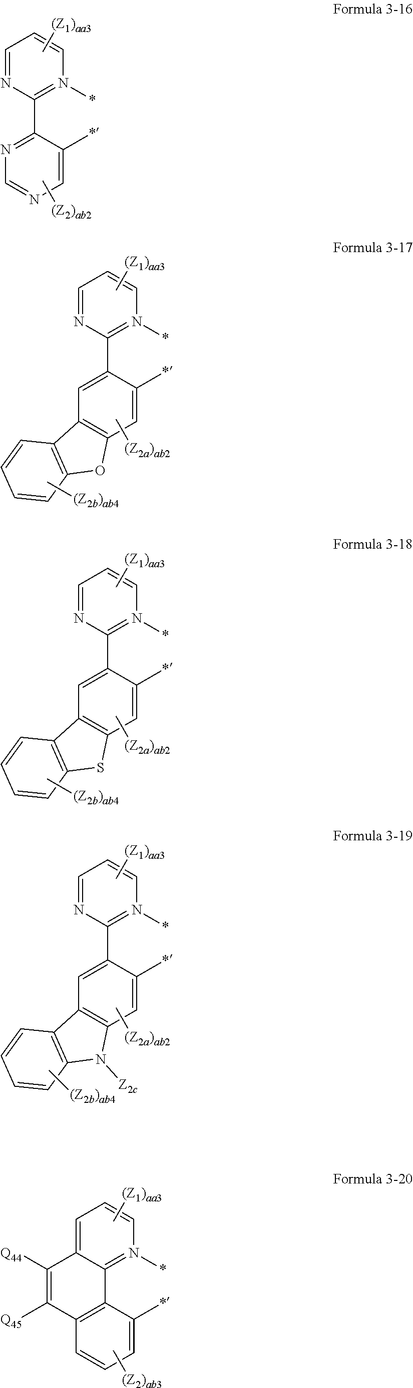

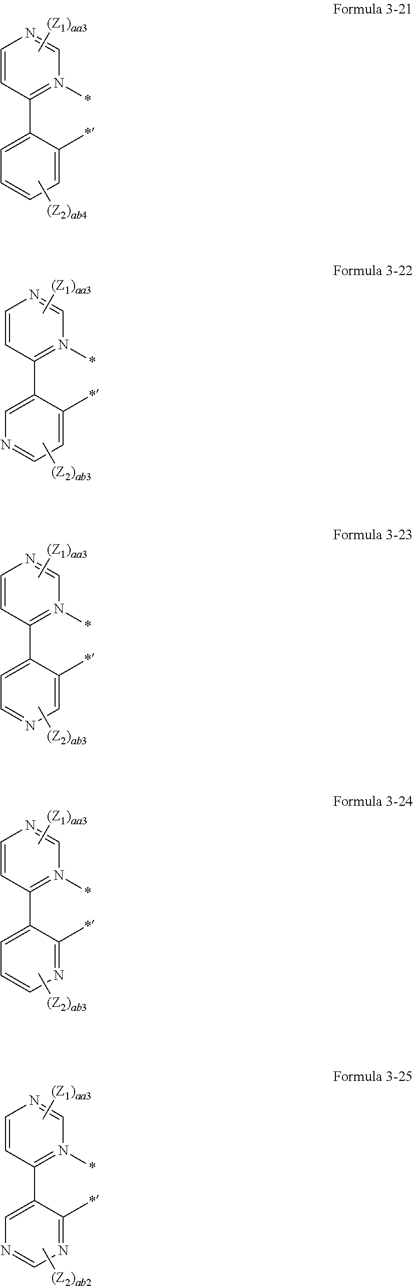

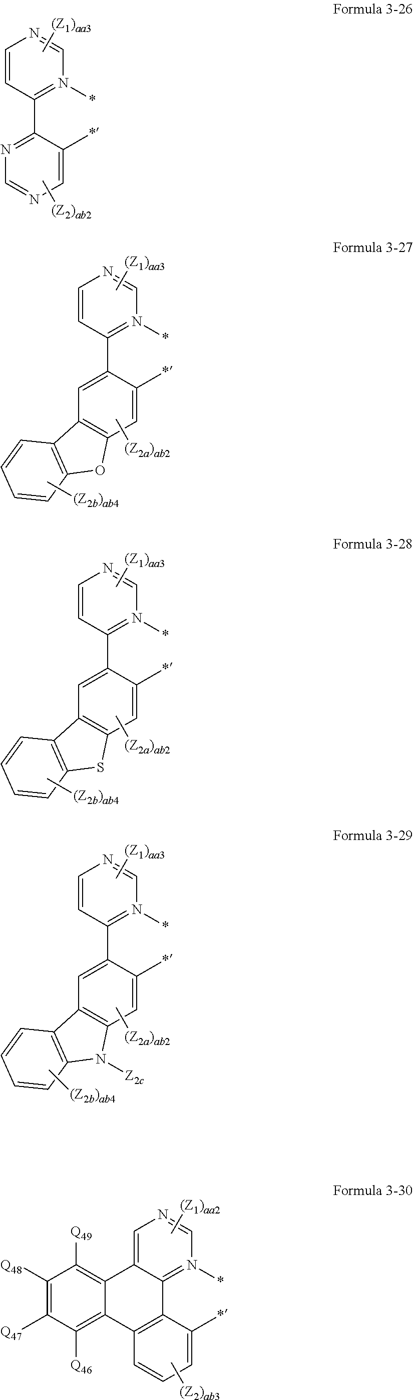

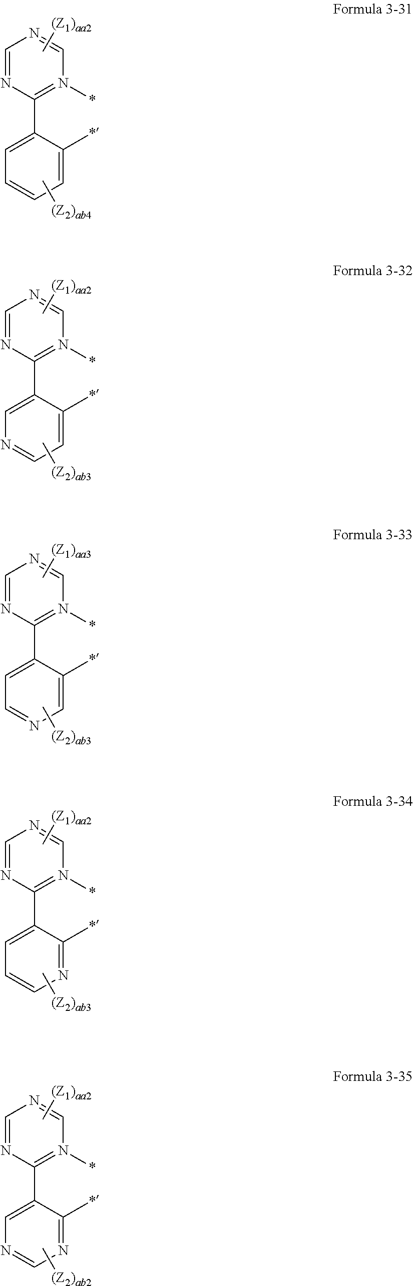

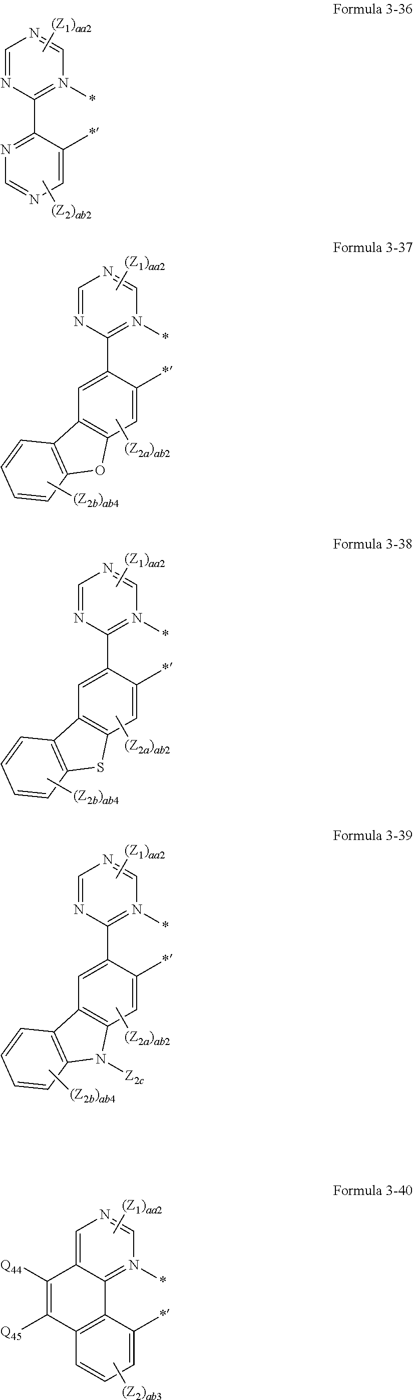

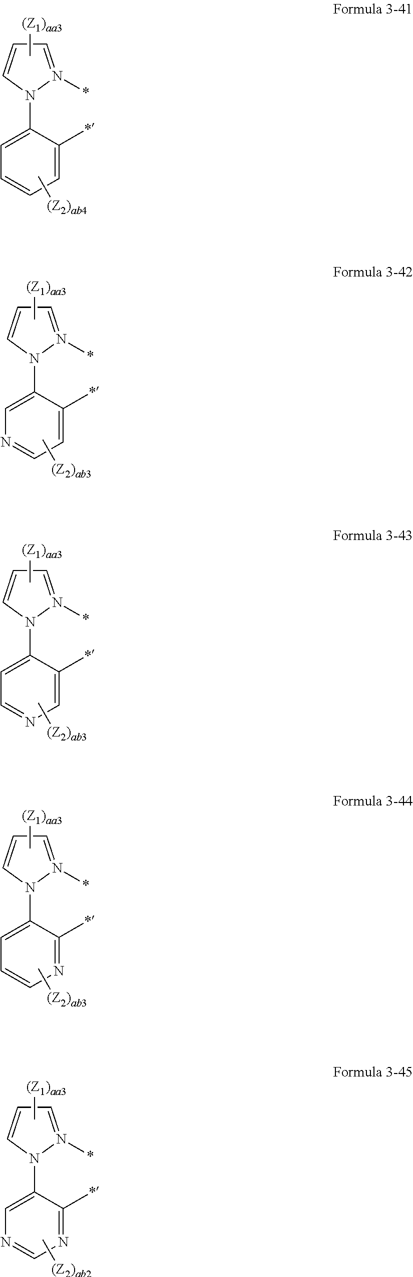

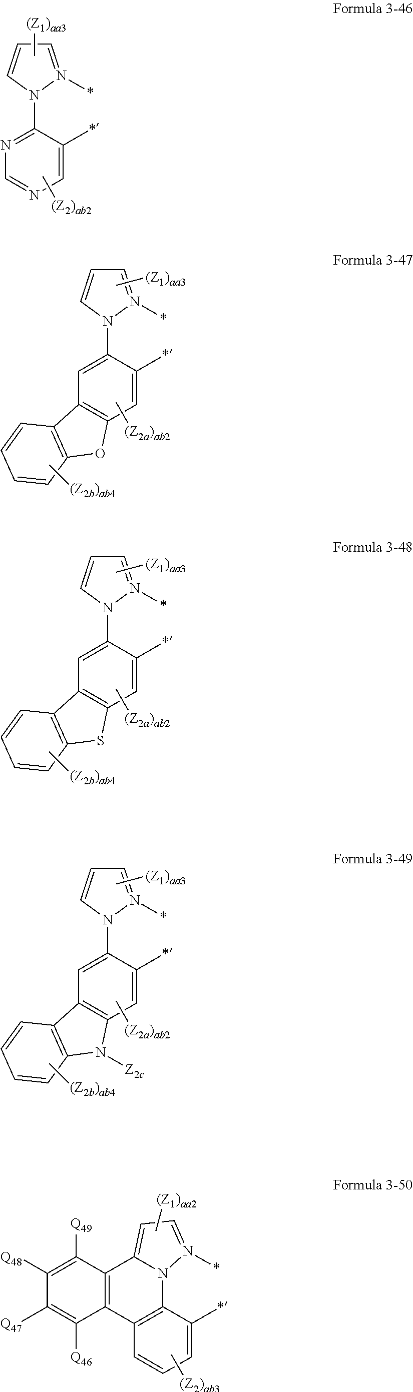

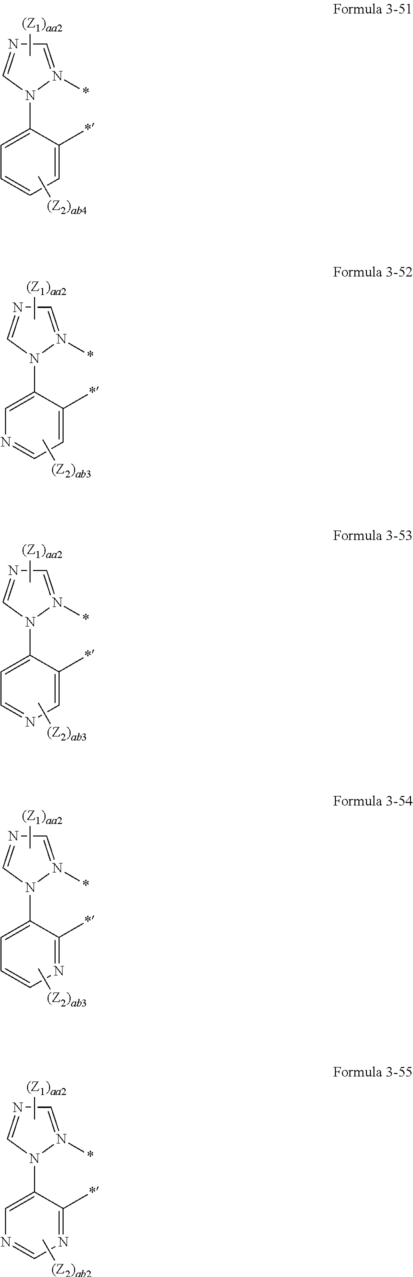

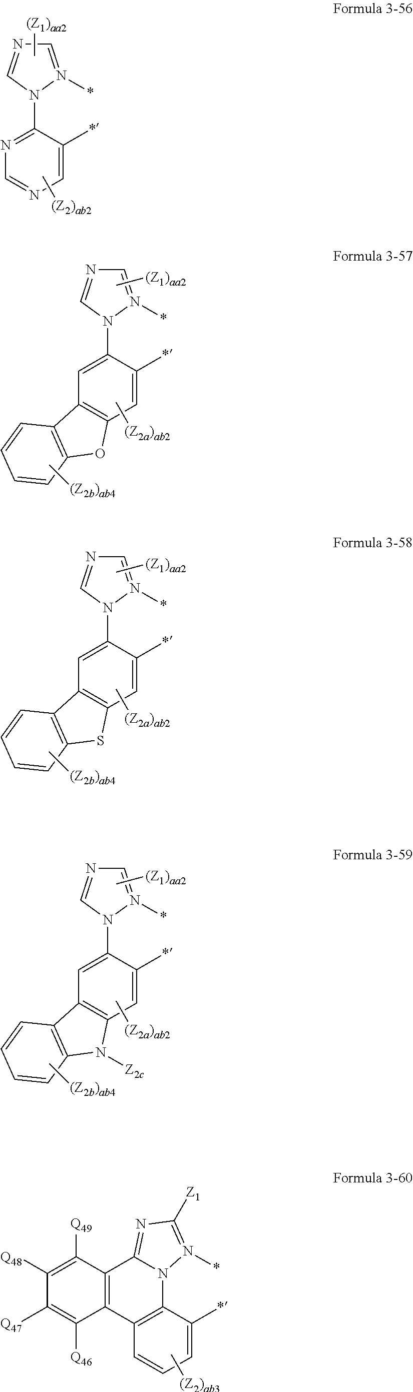

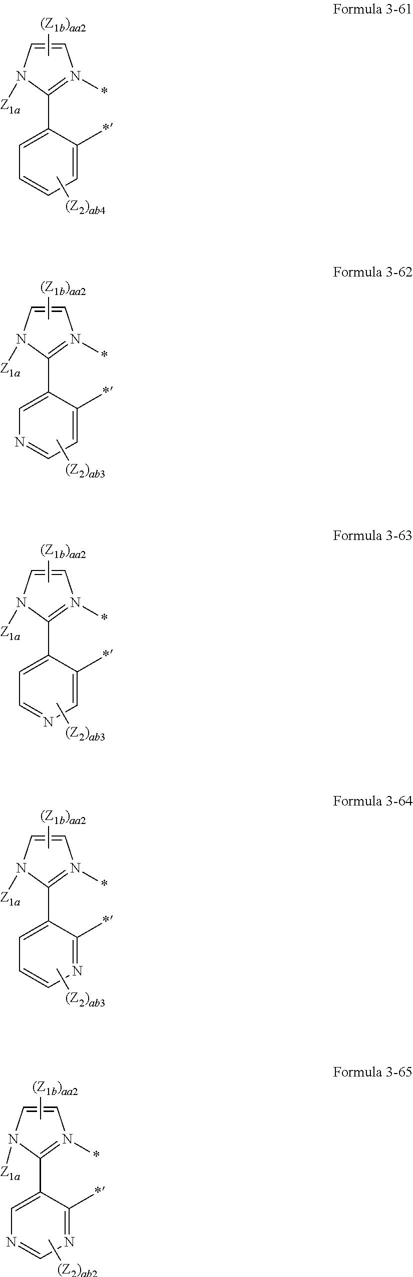

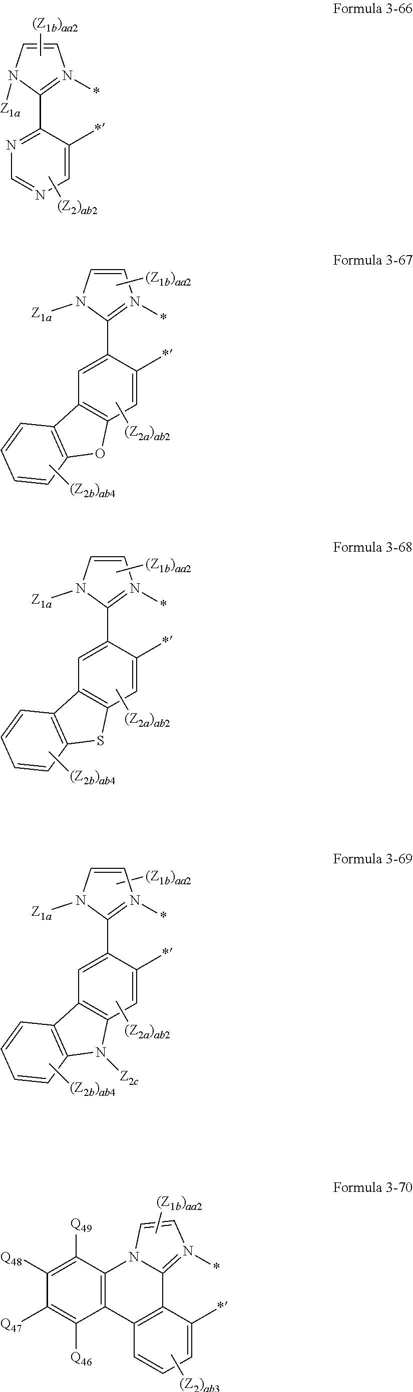

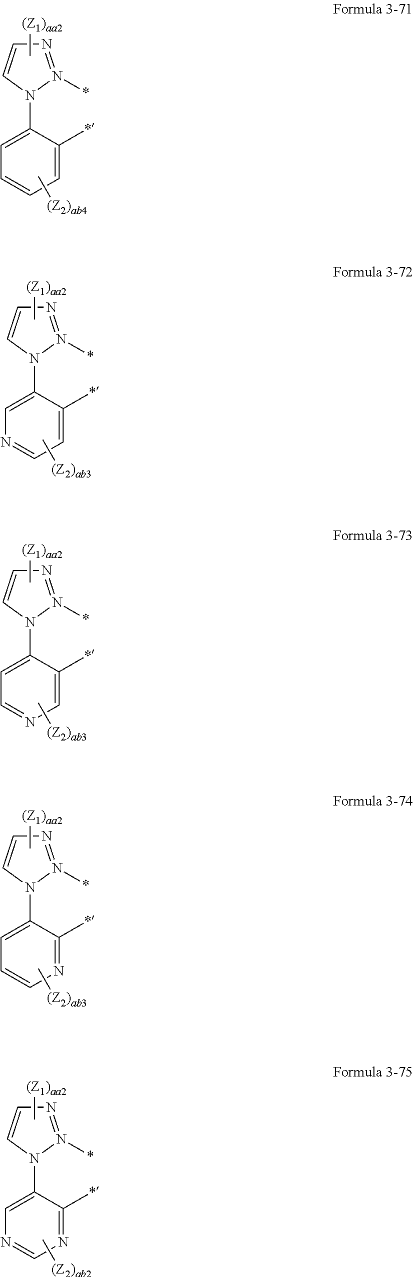

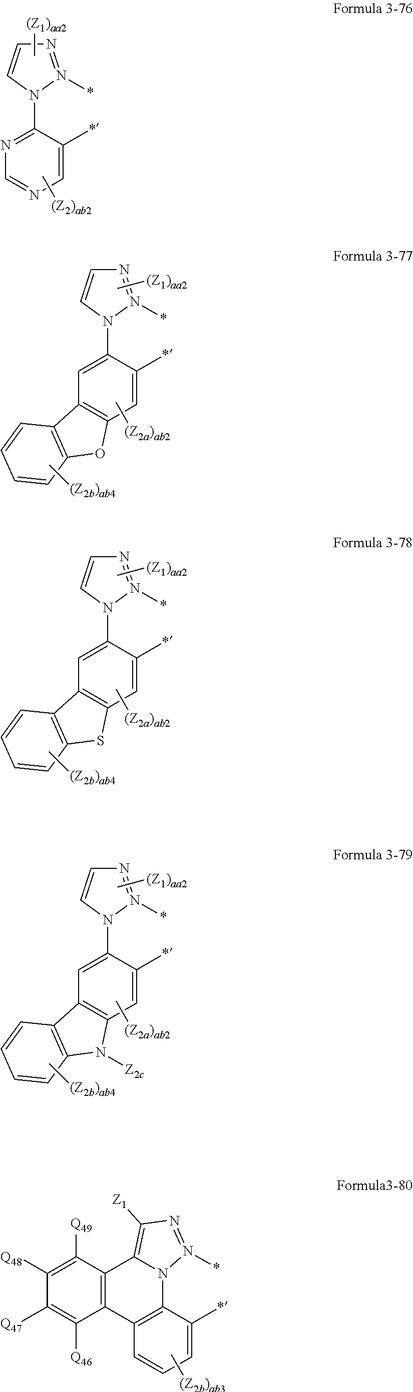

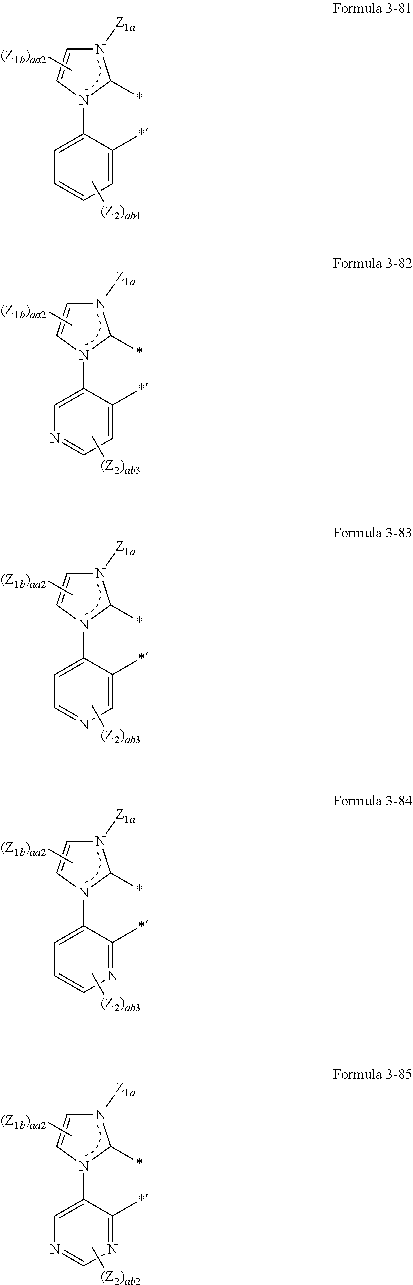

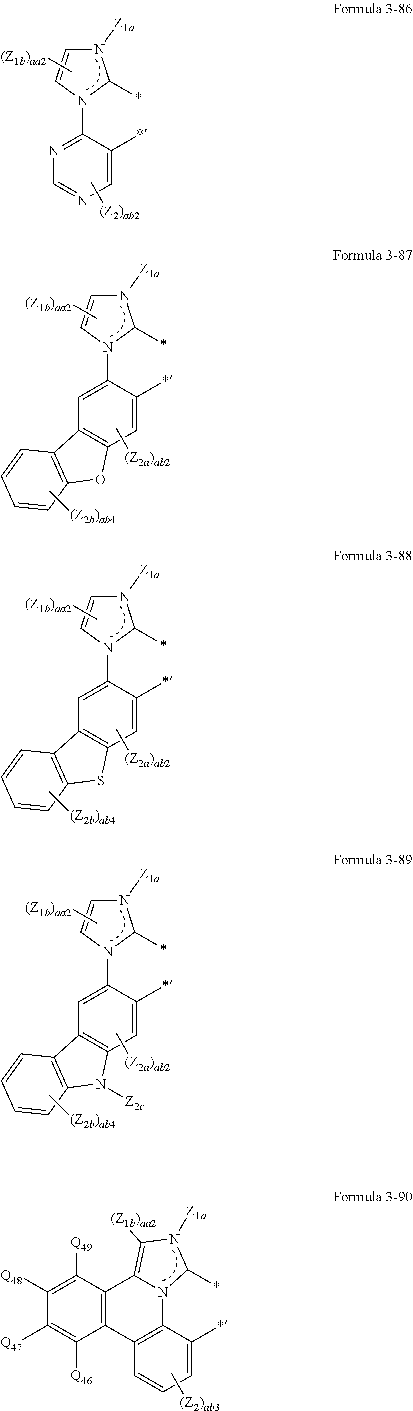

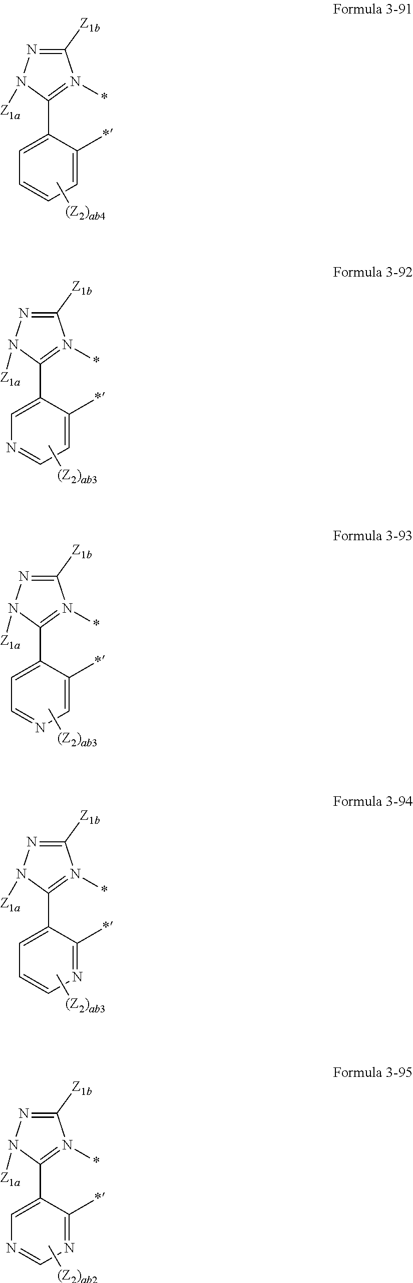

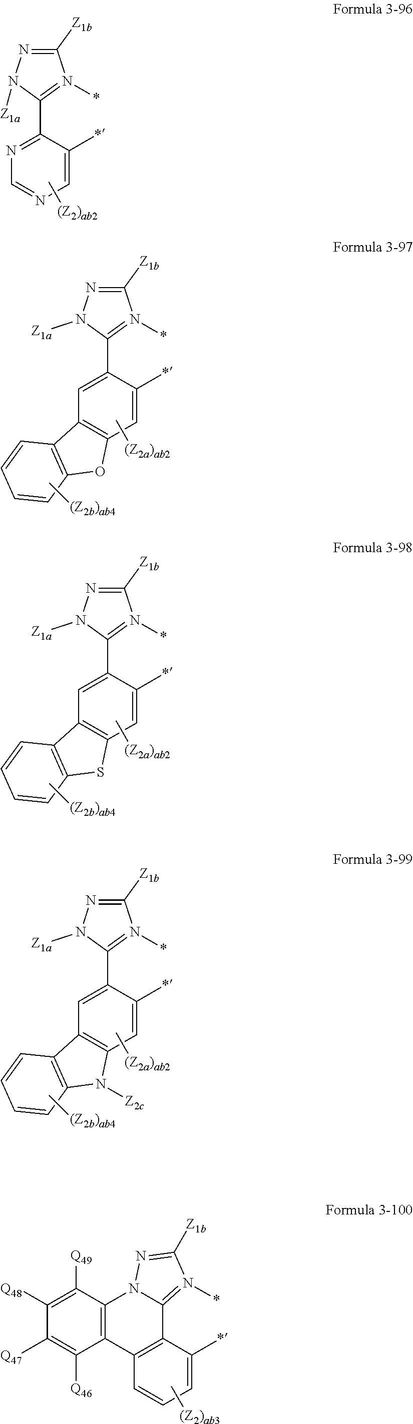

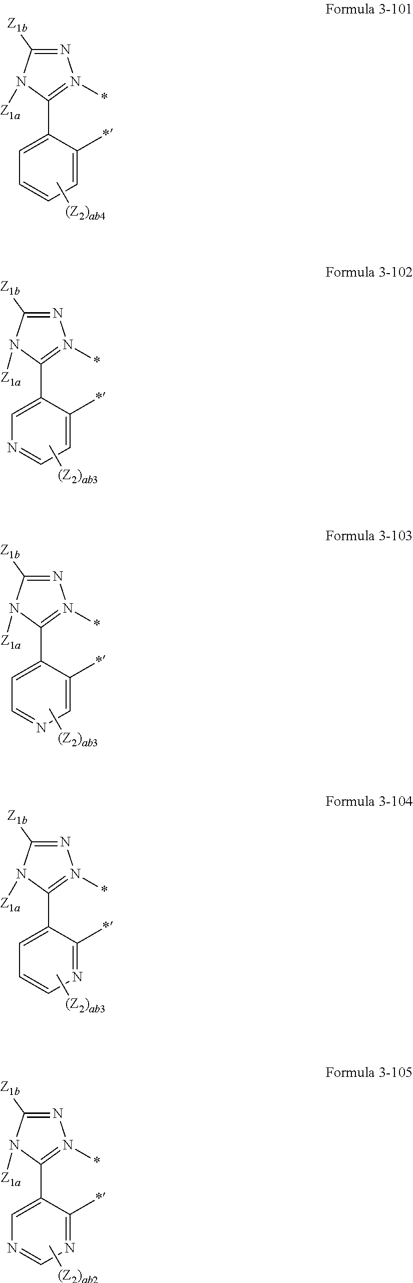

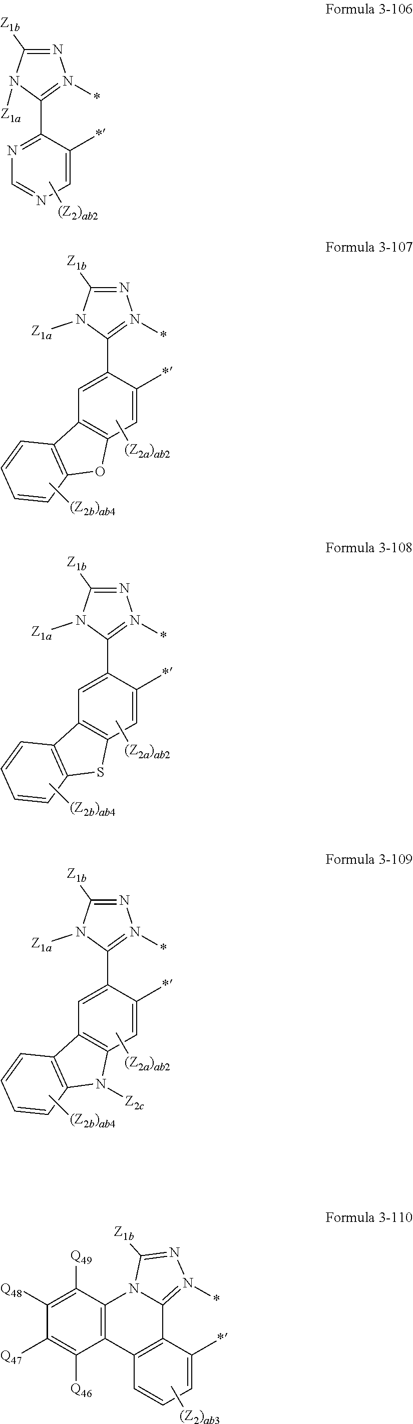

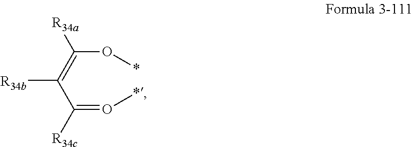

wherein, in Formulae 3-1 to 3-111,

Z₁, Z₂, Z₁ₐ, Z_{1b}, Z₂ₐ, Z_{2b}, Z_{2c}, R₃₄ₐ, R_{34b}, and R_{34c} may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group; and

—N(Q₁)(Q₂), —Si(Q₃)(Q₄)(Q₅), —B(Q₆)(Q₇), and —P(═O)(Q₈)(Q₉),

wherein Q₁ to Q₉ and Q₄₄ to Q₄₉ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium and a C₁-C₁₀ alkyl group,

aa2 and ab2 may each independently be 1 or 2,

aa3 and ab3 may each independently be selected from 1, 2, and 3,

aa4 and ab4 may each independently be selected from, 1, 2, 3, and 4, and

* and *′ each indicate a binding site to M in Formula 1.

Z₁, Z₂, Z₁ₐ, Z_{1b}, Z₂ₐ, Z_{2b}, Z_{2c}, R₃₄ₐ, R_{34b}, and R_{34c} in Formulae 3-1 to 3-111 may each independently be selected from:

hydrogen, deuterium, —F, a cyano group, a nitro group, —SF₅, a methyl group, an ethyl group, an n-propyl group, an iso-propyl group, an n-butyl group, an iso-butyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group;

a methyl group, an ethyl group, an n-propyl group, an iso-propyl group, an n-butyl group, an iso-butyl group, a sec-butyl group, a tert-butyl group, an n-pentyl group, an isopentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group, each substituted with at least one selected from deuterium, —F, —CD₃, —CD₂H, —CDH₂, —CF₃, —CF₂H, —CFH₂, a cyano group, a nitro group, a C₁-C₁₀ alkyl group, a C₁-C₁₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group; and

—N(Q₁)(Q₂), —Si(Q₃)(Q₄)(Q₅), —B(Q₆)(Q₇), and —P(═O)(Q₈)(Q₉),

wherein Q₁ to Q₉ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium and a C₁-C₁₀ alkyl group,

but embodiments are not limited thereto.

In one or more embodiments, L₂ in Formula 1 may be selected from ligands represented by Formulae 3-1 (1) to 3-1 (59) and 3-111:

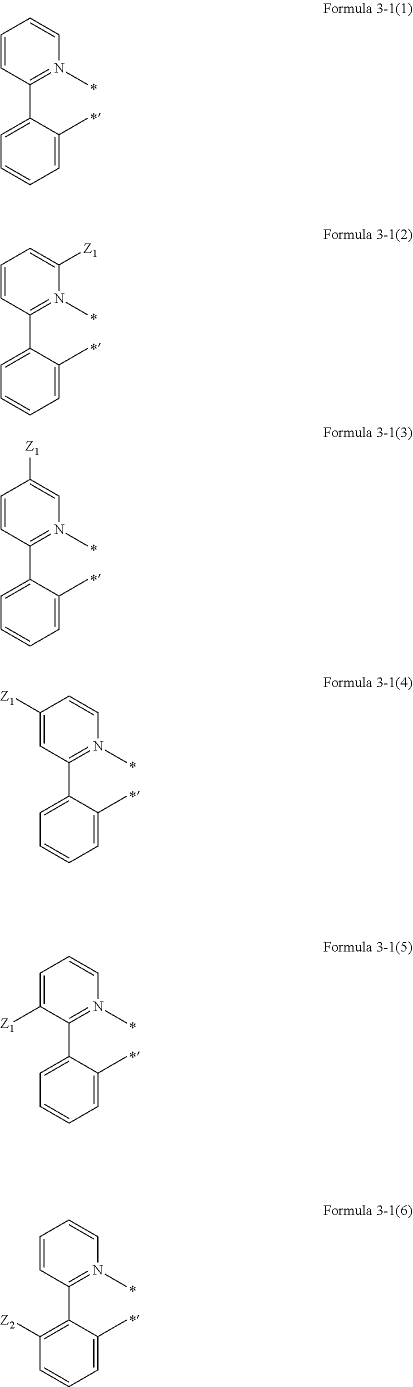

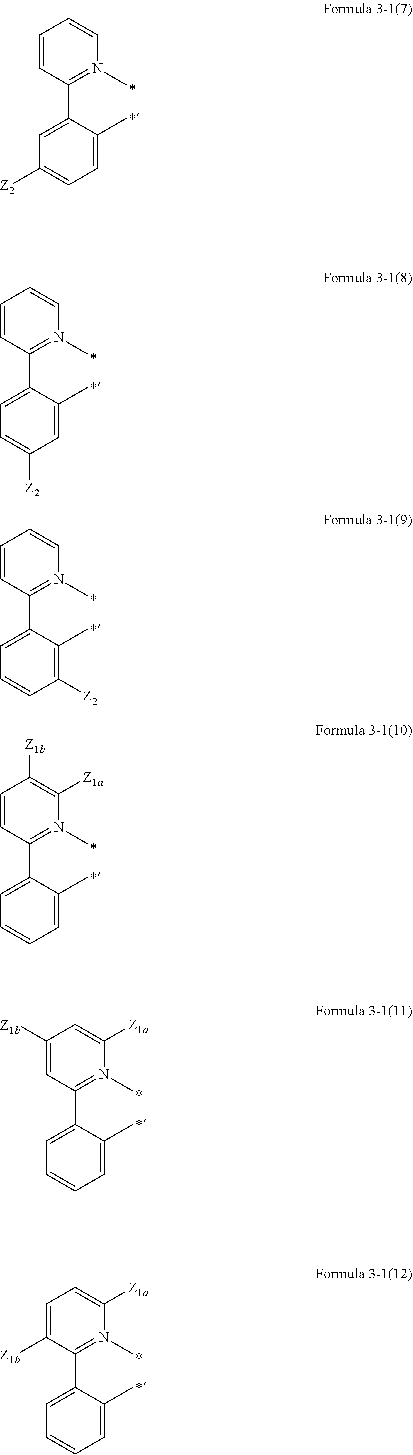

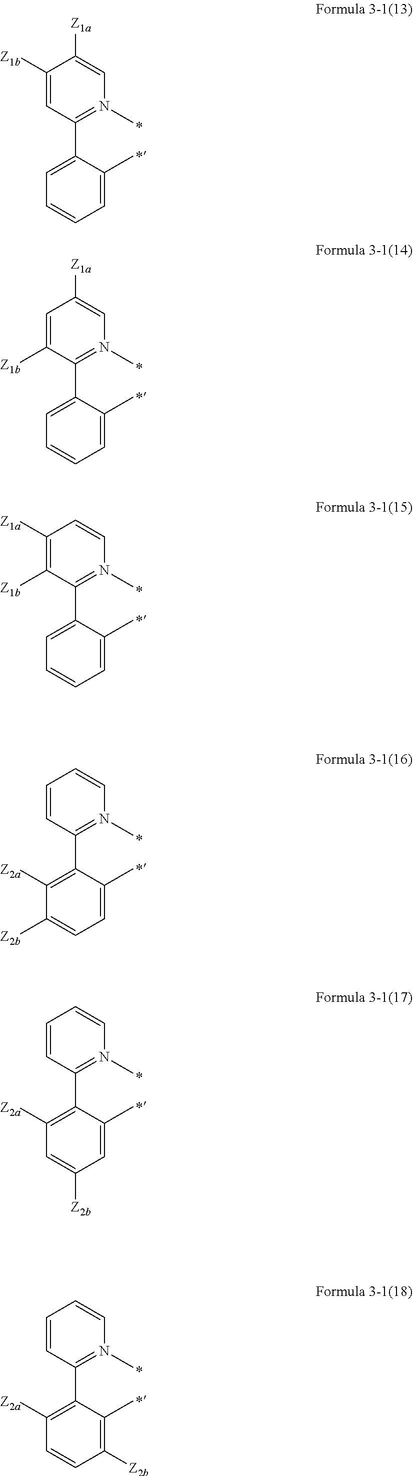

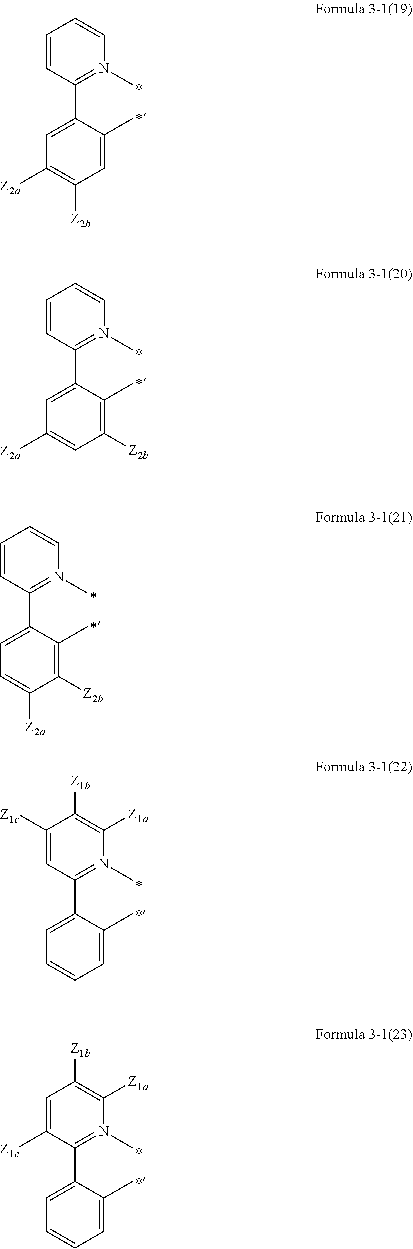

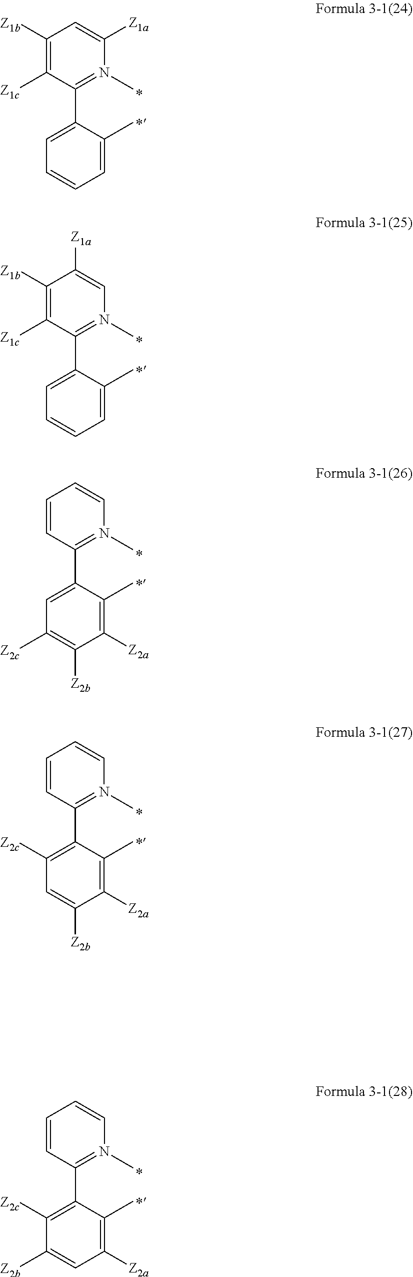

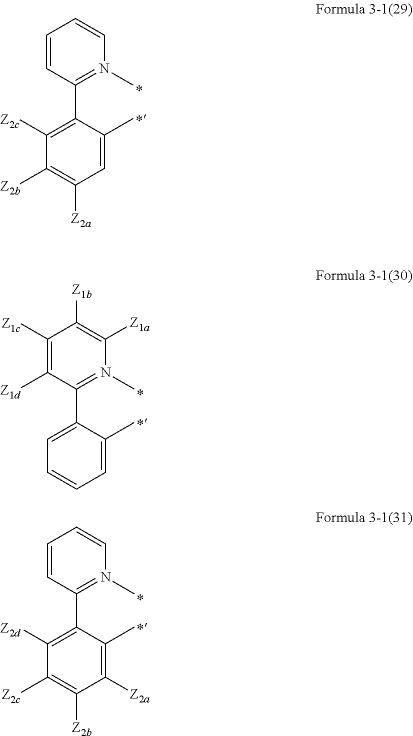

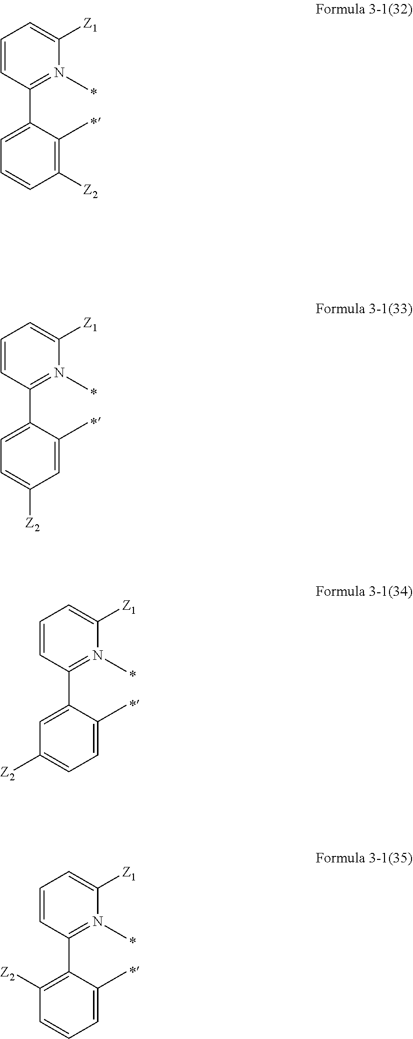

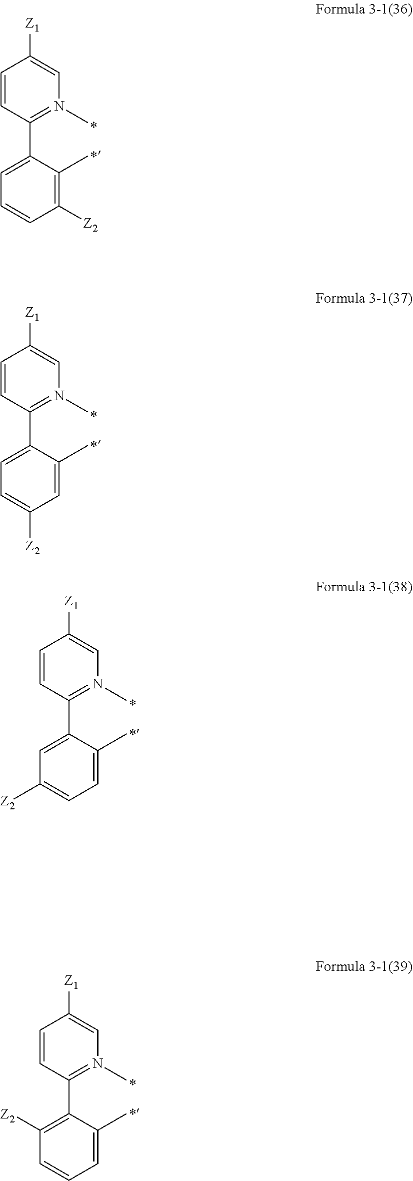

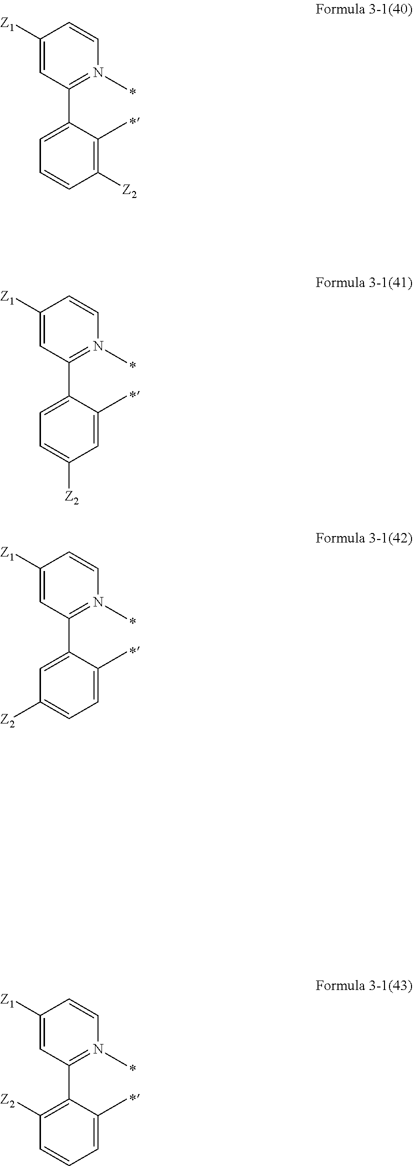

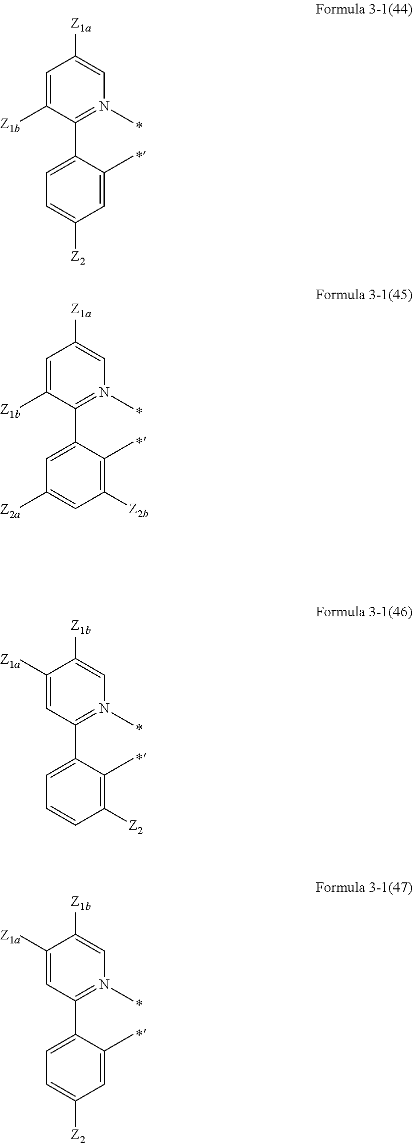

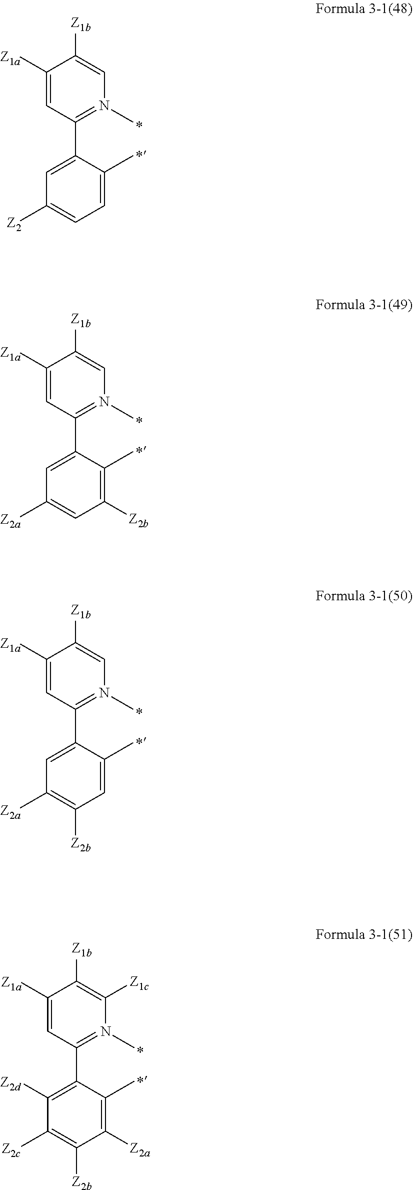

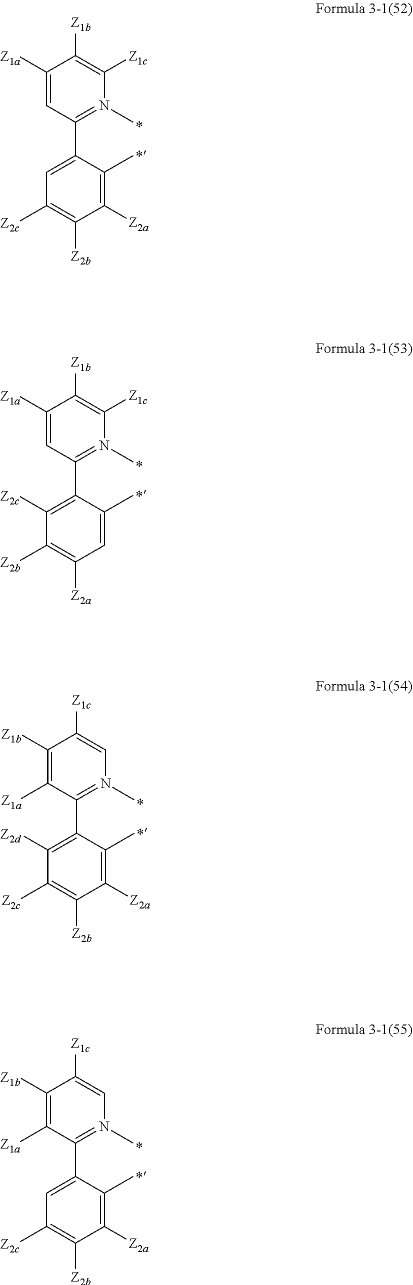

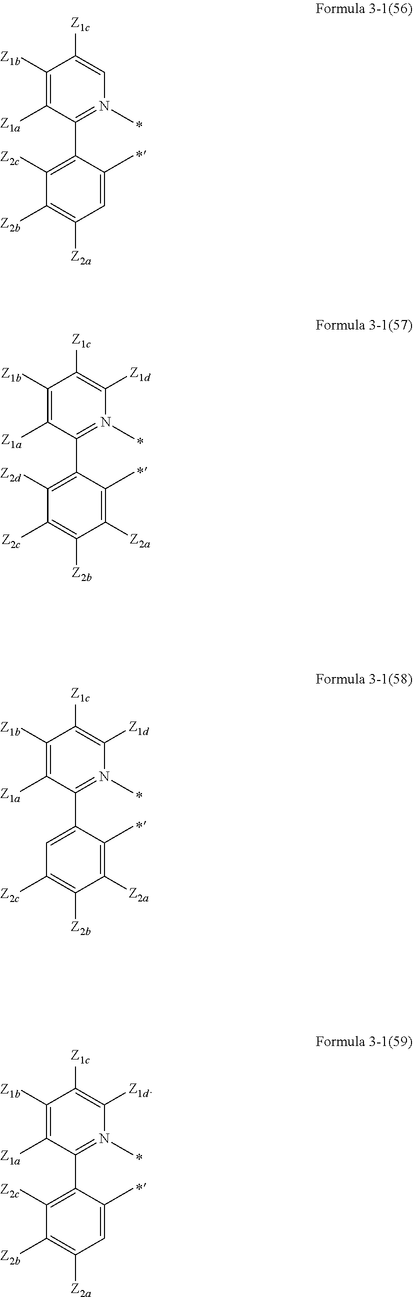

In one or more embodiments, in Formulae 3-1(1) to 3-1(59) and 3-111,

Z₁, Z₂, Z₁ₐ, Z_{1b}, Z_{1c}, Z_{1d}, Z₂ₐ, Z_{2b}, Z_{2c}, Z_{2d}, R₃₄ₐ, R_{34b}, and R_{34c} may each independently be selected from deuterium, —F, a cyano group, a nitro group, —SF₅, —CH₃, —CD₃, —CD₂H, —CDH₂, —CF₃, —CF₂H, —CFH₂, —Si(Q₃)(Q₄)(Q₅), groups represented by Formulae 9-1 to 9-17, and groups represented by Formulae 10-1 to 10-30,

wherein Q₃ to Q₅ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium and a C₁-C₁₀ alkyl group, and

* and *′ each indicate a binding site to M in Formula 1.

In one or more embodiments, in Formula 1, M may be Ir and n1+n2 may be 3; or M may Pt and n1+n2 may be 2, the organometallic compound represented by Formula 1 may be electrically neutral, L₁ in Formula 1 may be selected from ligands represented by Formulae 2A-1 to 2A-16, 2AA-1, 2AA-2, 2AA-3, 2AA-4, and 2AB, but embodiments are not limited thereto.

In one or more embodiments, in Formula 1, M may be Ir and n1+n2 may be 3; or M may be Pt and n1+n2 may be 2, the organometallic compound represented by Formula 1 may be electrically neutral, and L₁ in Formula 1 may be selected from ligands represented by Formulae 2A-1, 2A-5, 2AA-1, and 2AB, but embodiments are not limited thereto.

In one or more embodiments, in Formula 1, M may be Ir and n1+n2 may be 3; or M may be Pt and n1+n2 may be 2, the organometallic compound represented by Formula 1 may be electrically neutral, L₁ in Formula 1 may be selected from ligands represented by Formulae 2A-1, 2A-5, 2AA-1, and 2AB, and L₂ may be selected from ligands represented by Formulae 3-1 to 3-111 (for example, ligands represented by Formulae 3-1(1) to 3-1 (59) and 3-111), but embodiments are not limited thereto.

In one or more embodiments, in Formula 1, M may be Ir and the sum of n1 and n2 may be 3; or M may be Pt and the sum of n1 and n2 may be 2, the organometallic compound represented by Formula 1 may be electrically neutral, and L₁ in Formula 1 may be selected from ligands represented by Formulae 2A(1) to 2A(40), but embodiments are not limited thereto.

In one or more embodiments, in Formula 1, M may be Ir and the sum of n1 and n2 may be 3; or M may be Pt and the sum of n1 and n2 may be 2, the organometallic compound represented by Formula 1 may be electrically neutral, L₁ in Formula 1 may be selected from ligands represented by Formulae 2A(1) to 2A(40), and L₂ in Formula 1 is selected from ligands represented by Formulae 3-1 to 3-111 (for example, ligands represented by Formulae 3-1(1) to 3-1(59) and 3-111), but embodiments are not limited thereto.

In one or more embodiments, the organometallic compound represented by Formula 1 may be one selected from Compounds 1 to 564, but is not limited thereto.

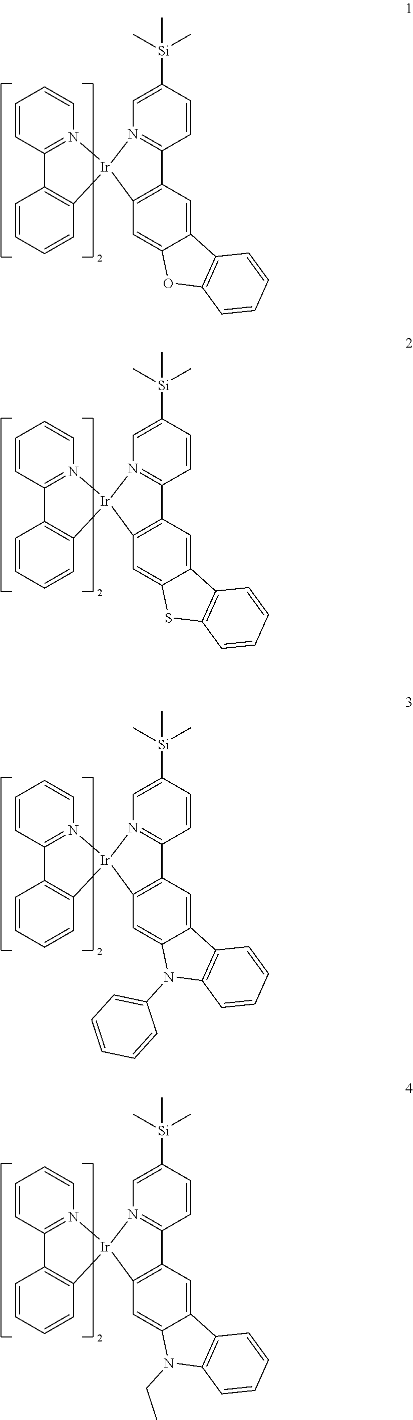

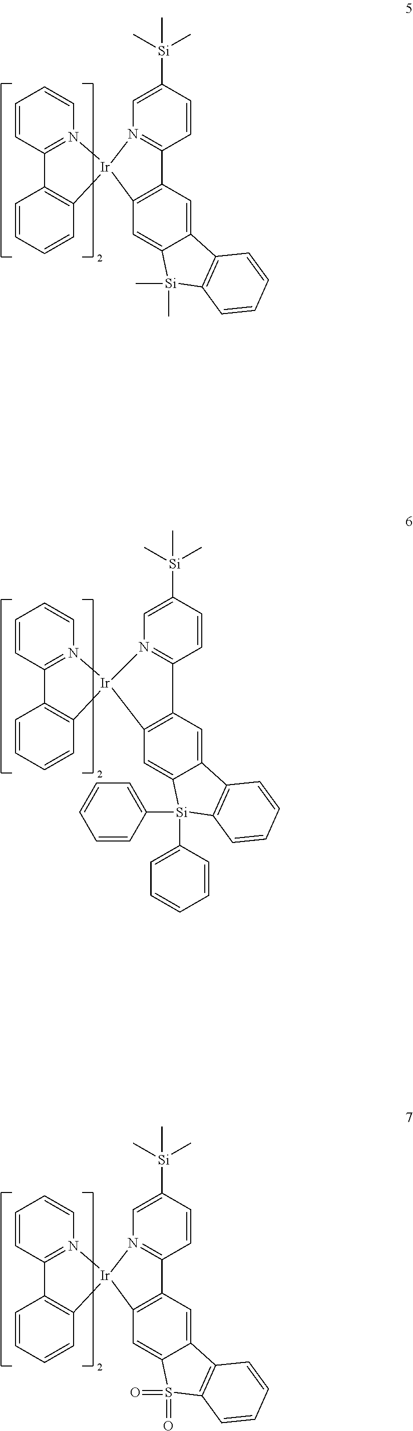

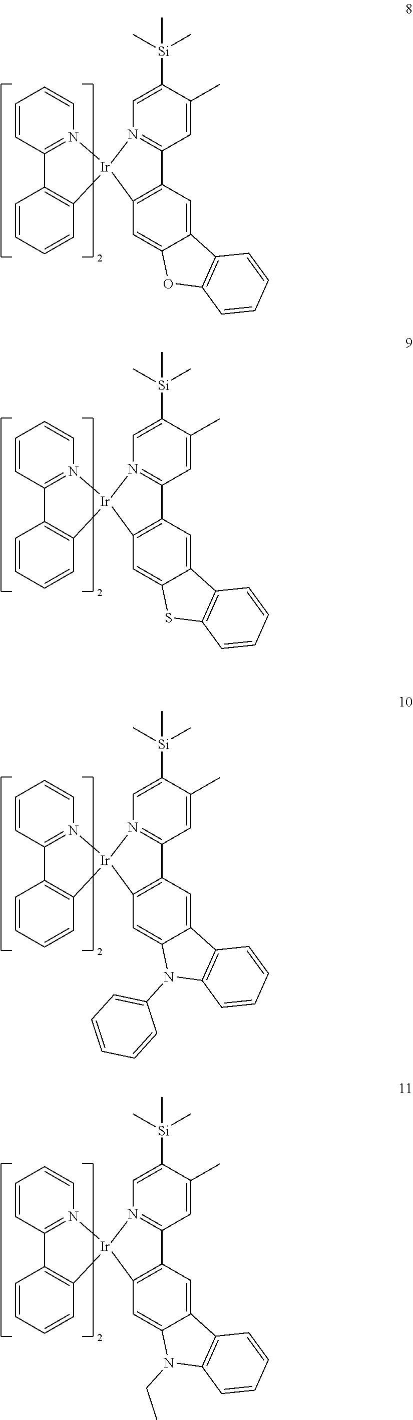

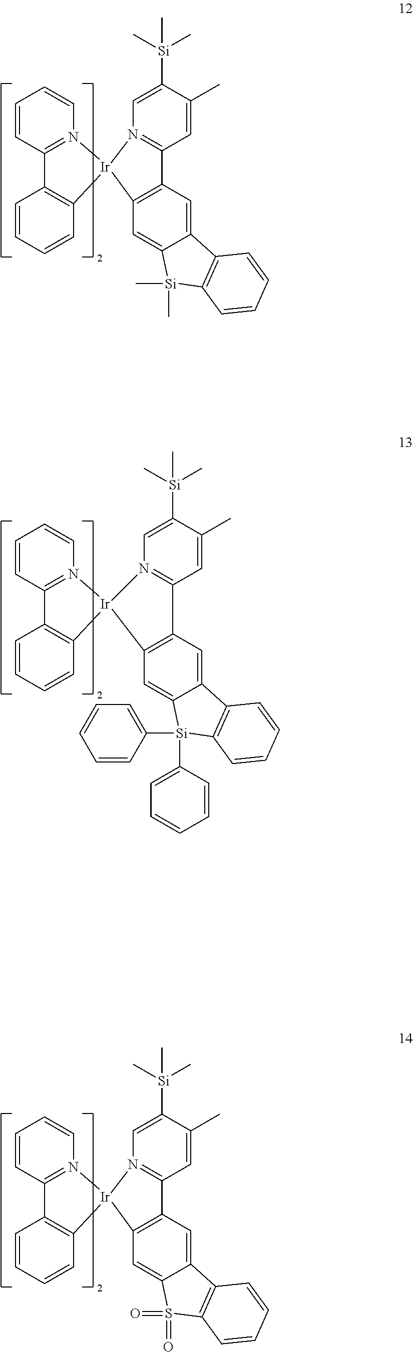

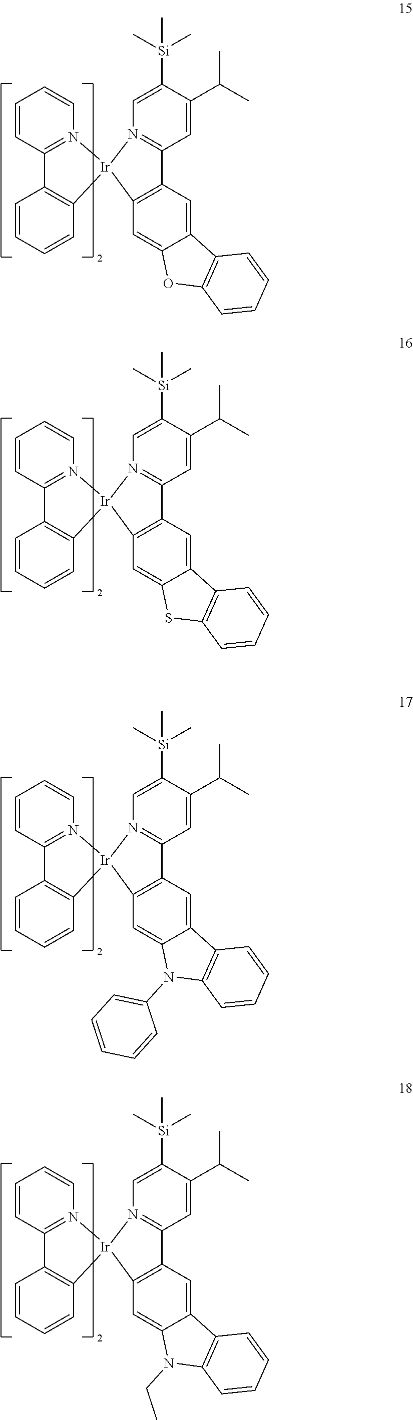

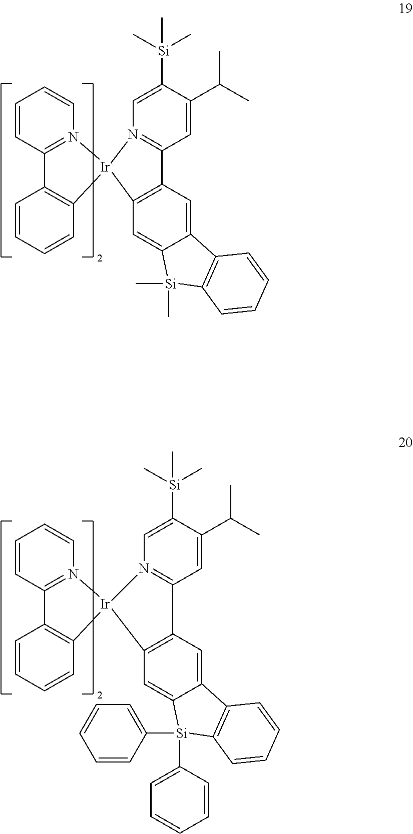

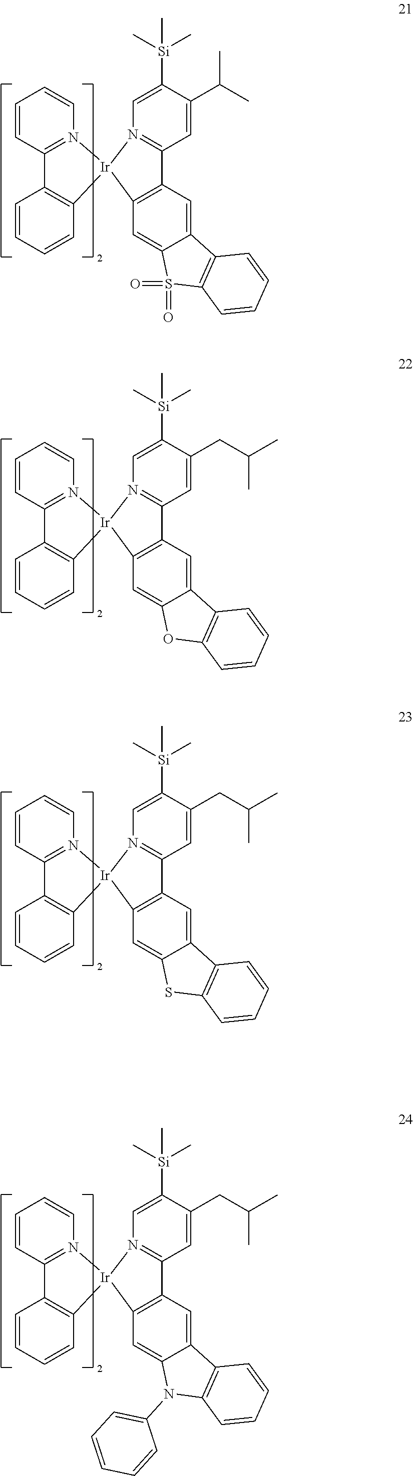

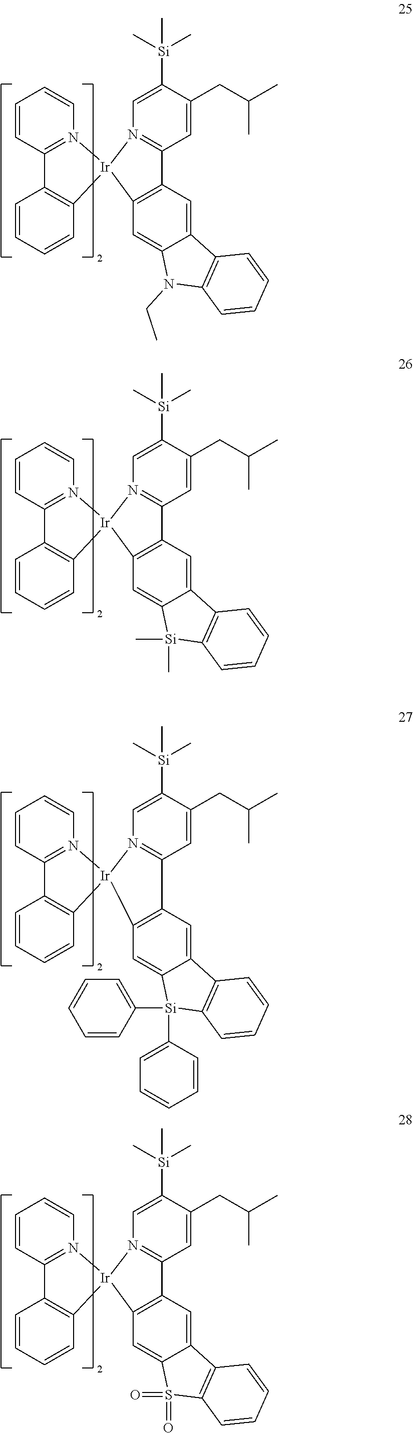

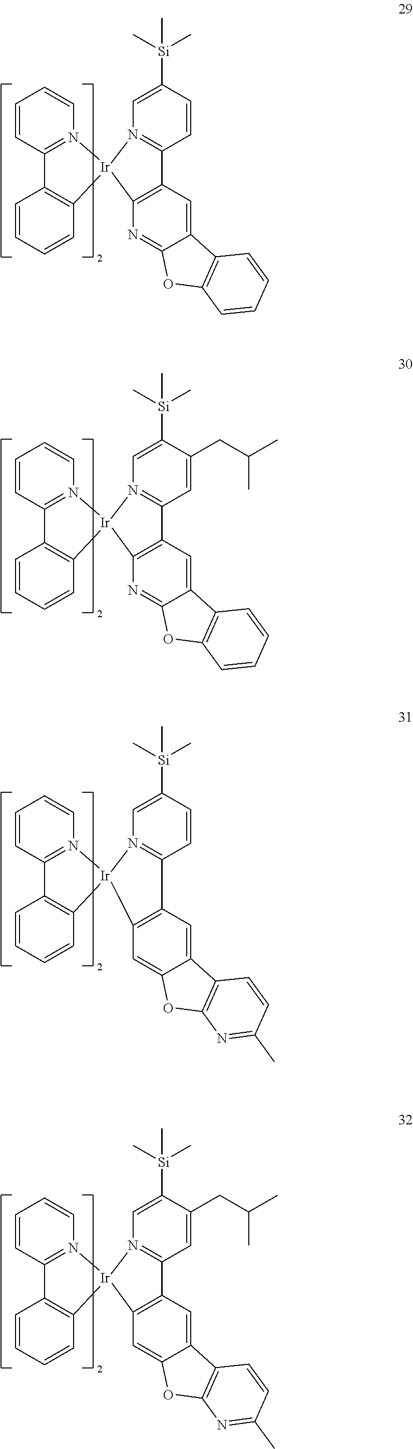

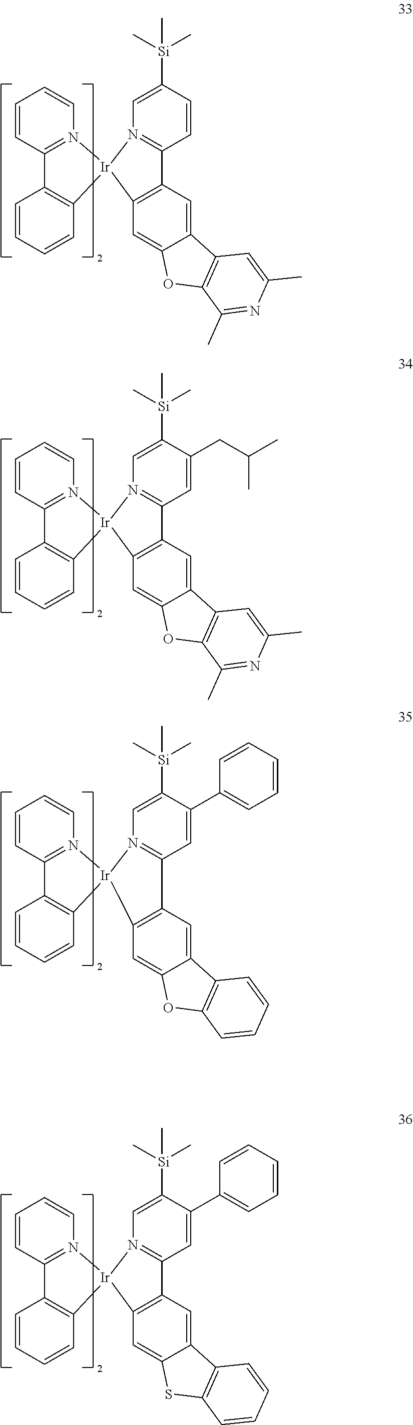

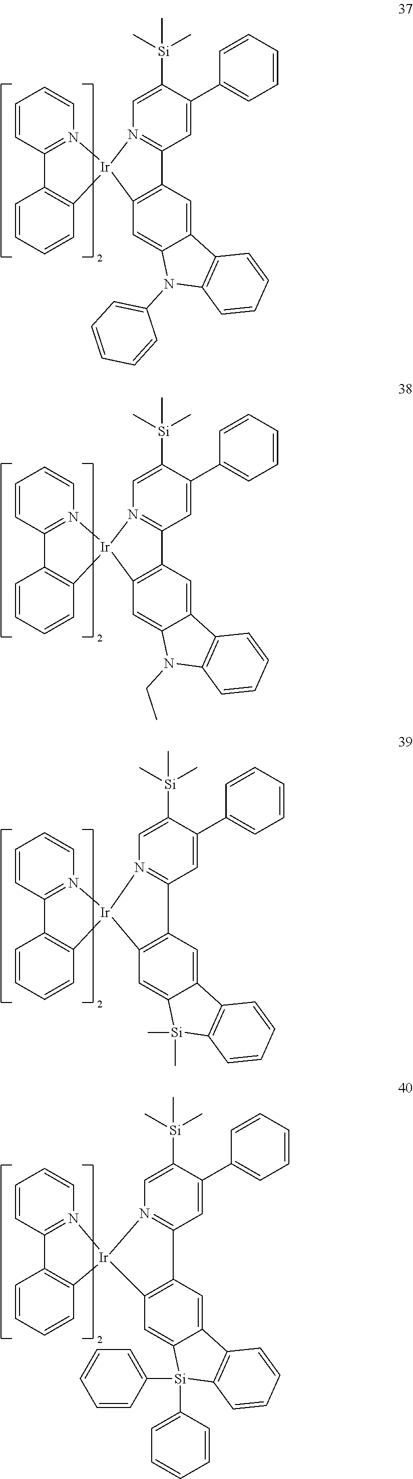

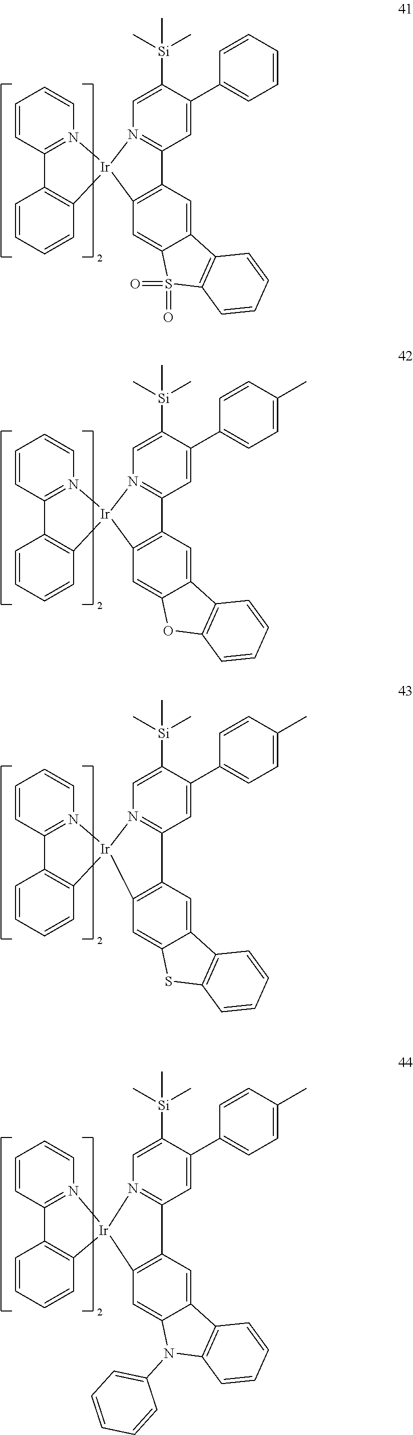

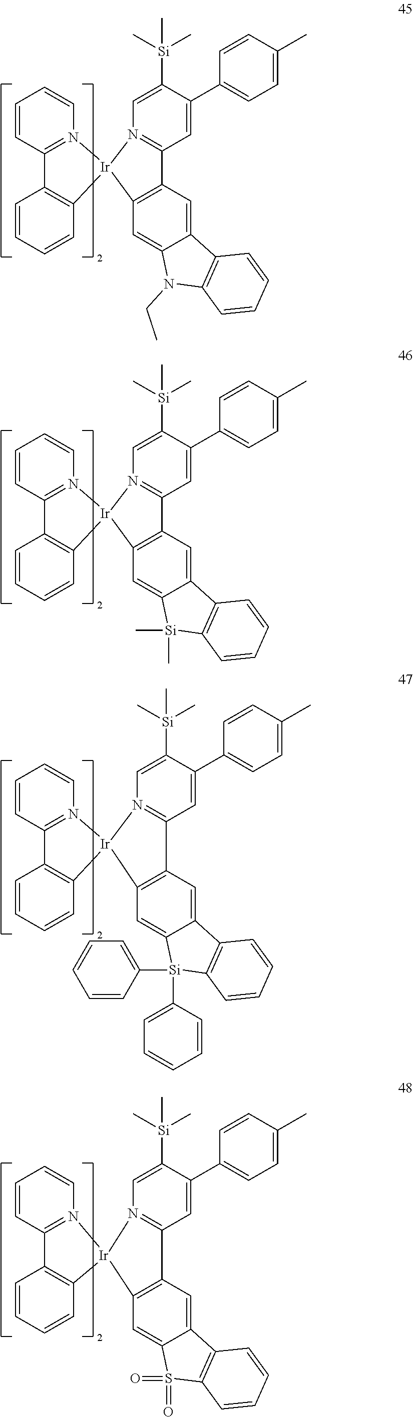

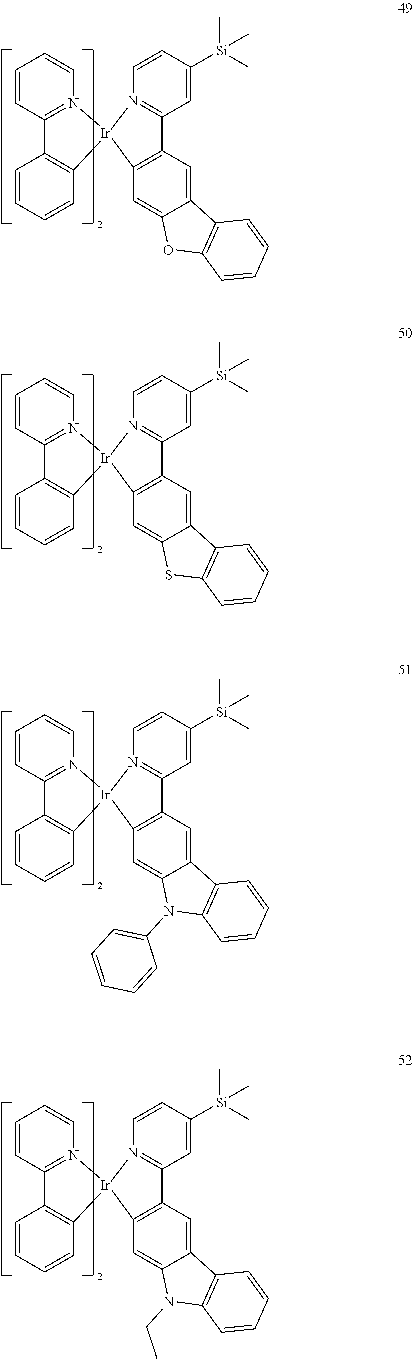

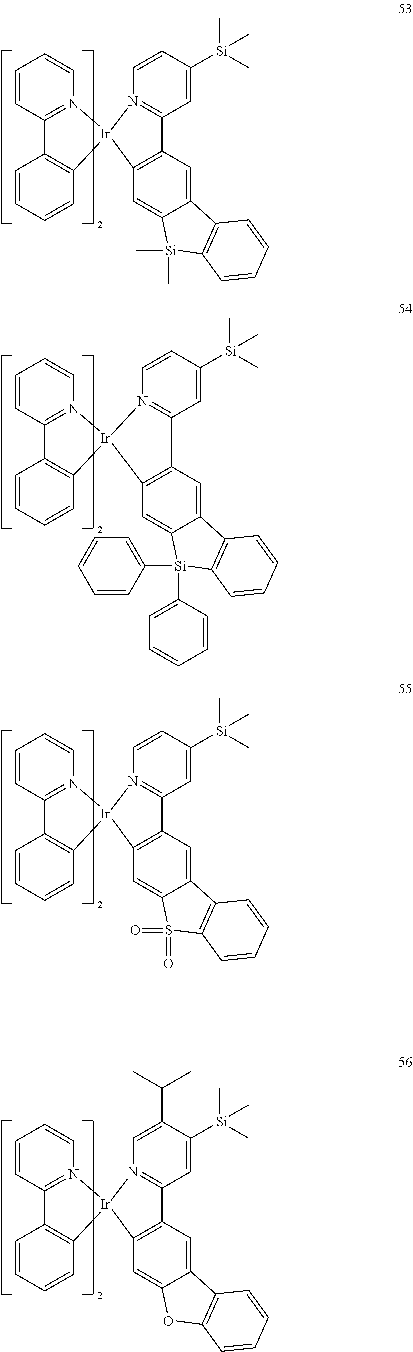

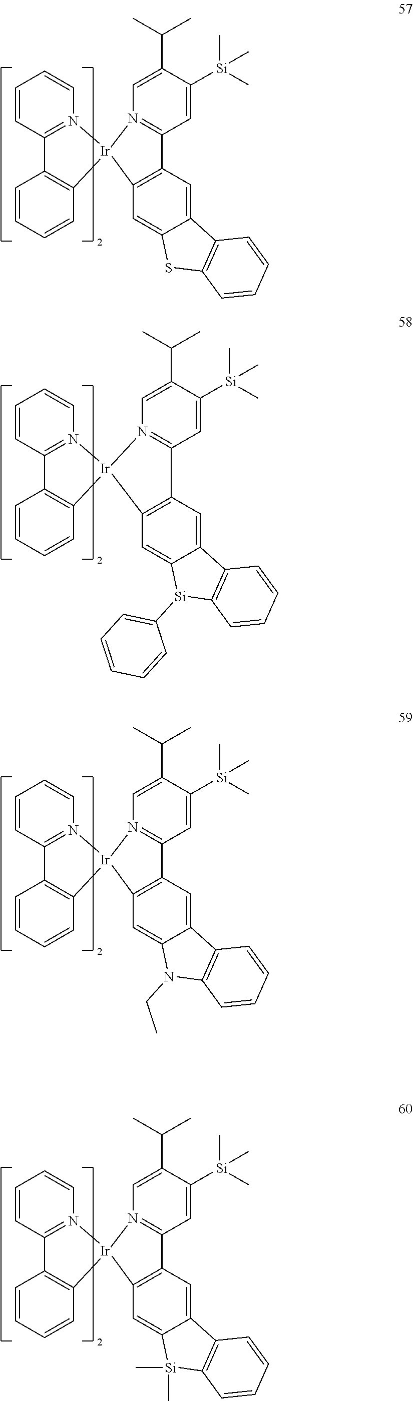

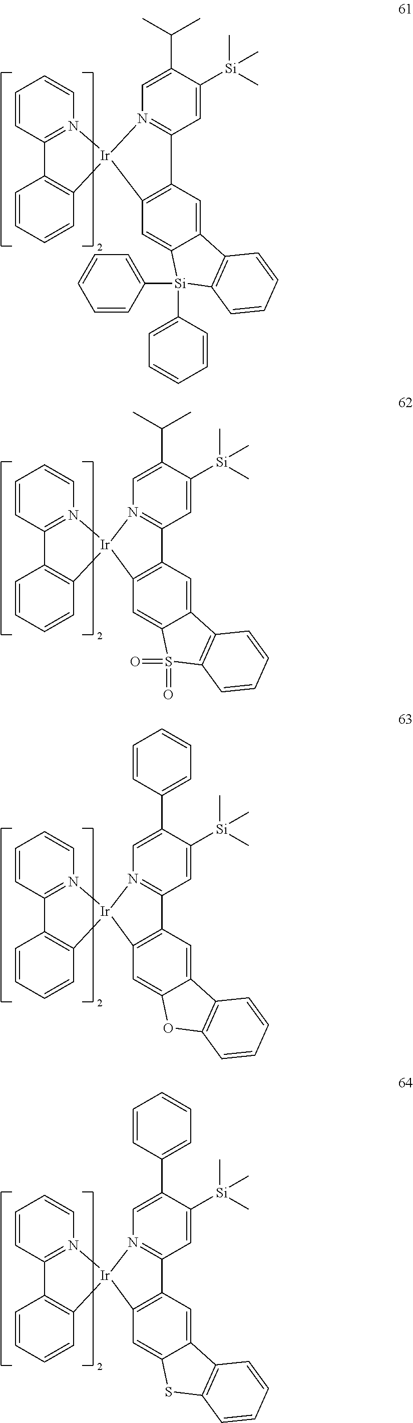

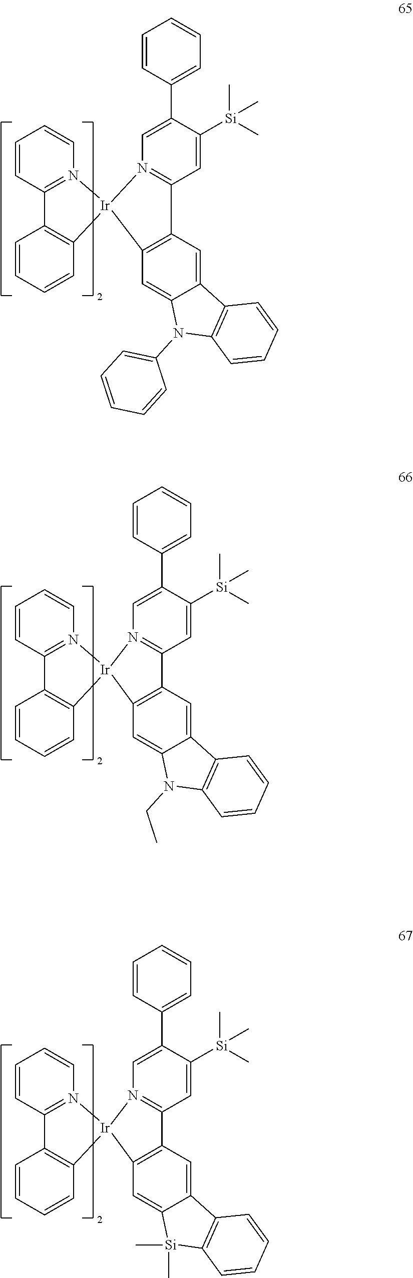

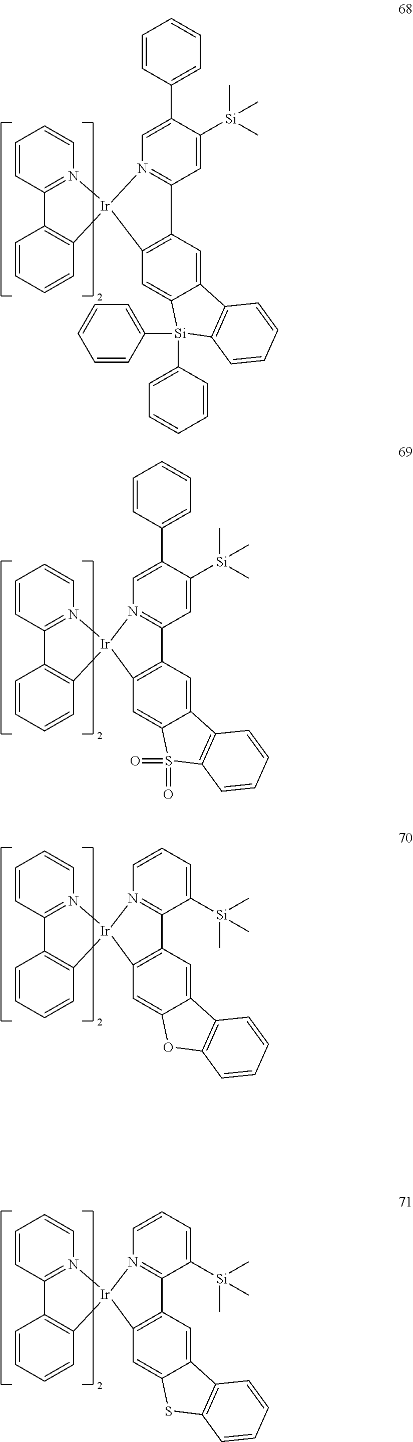

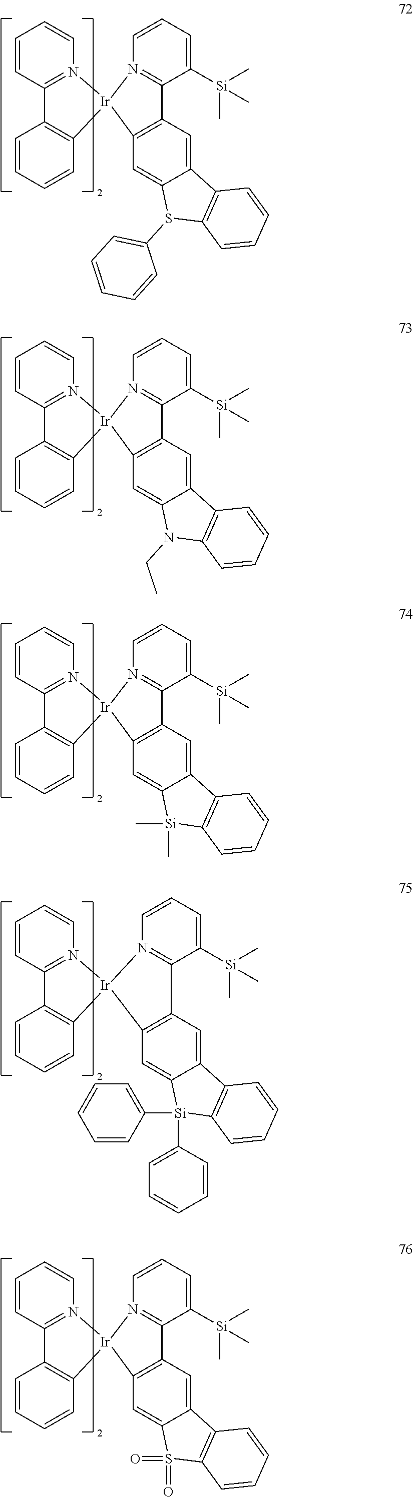

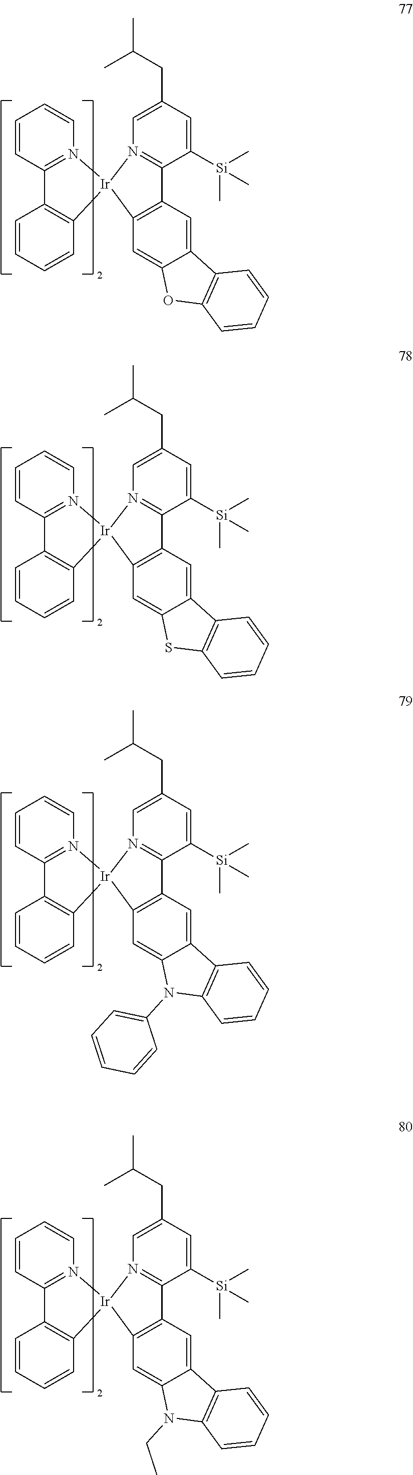

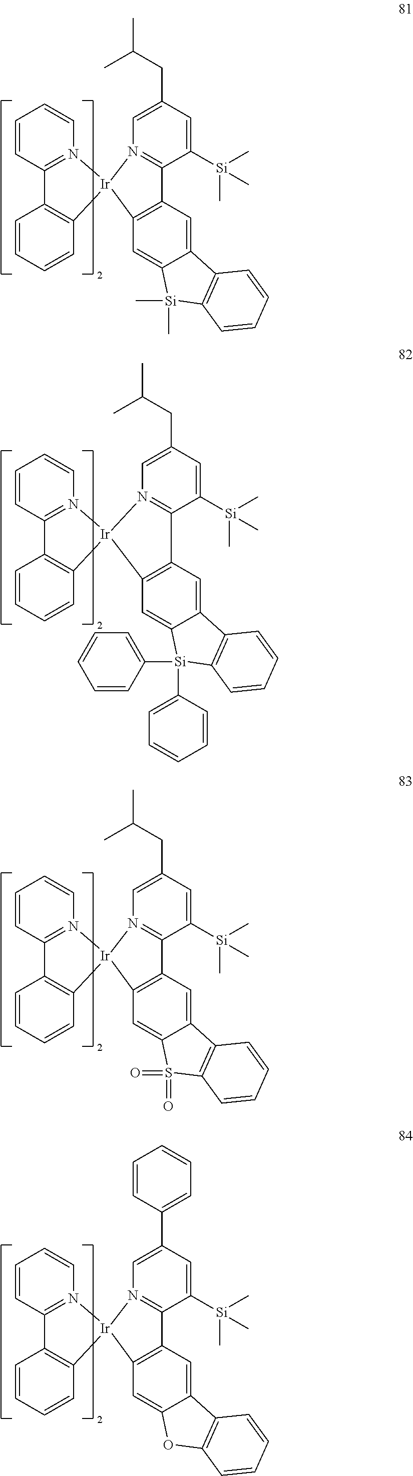

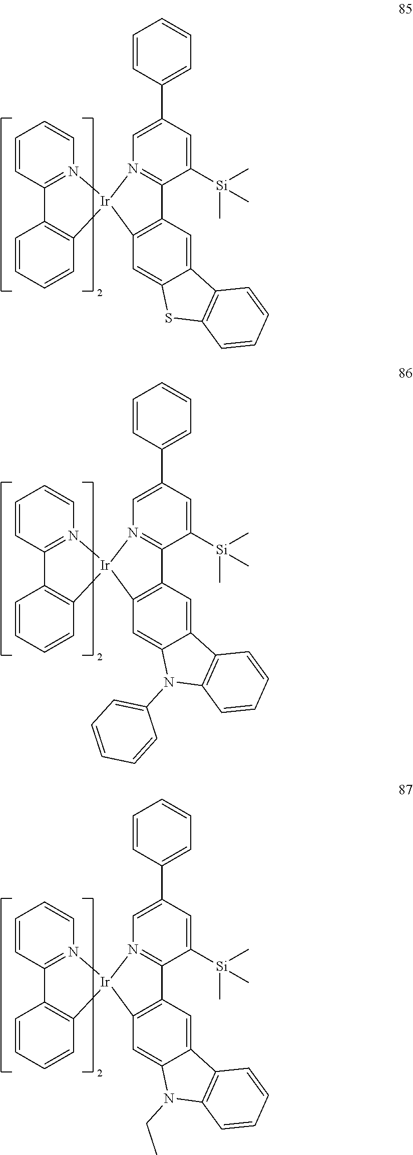

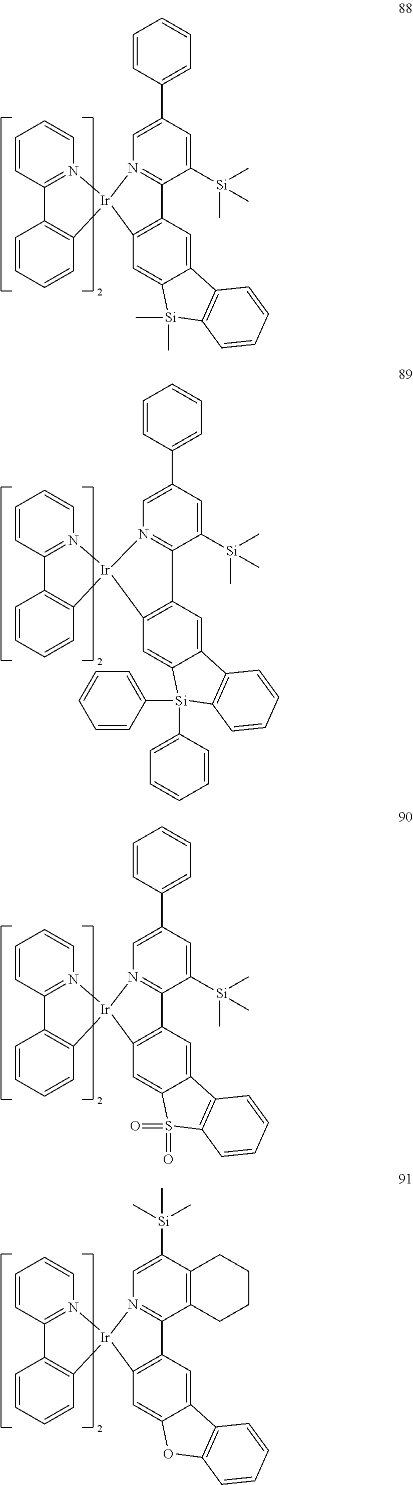

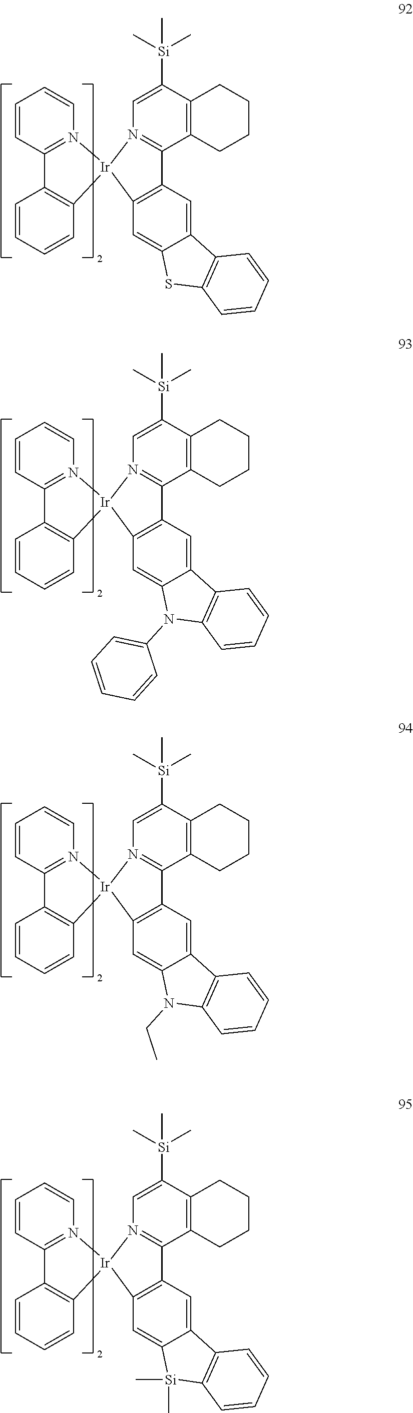

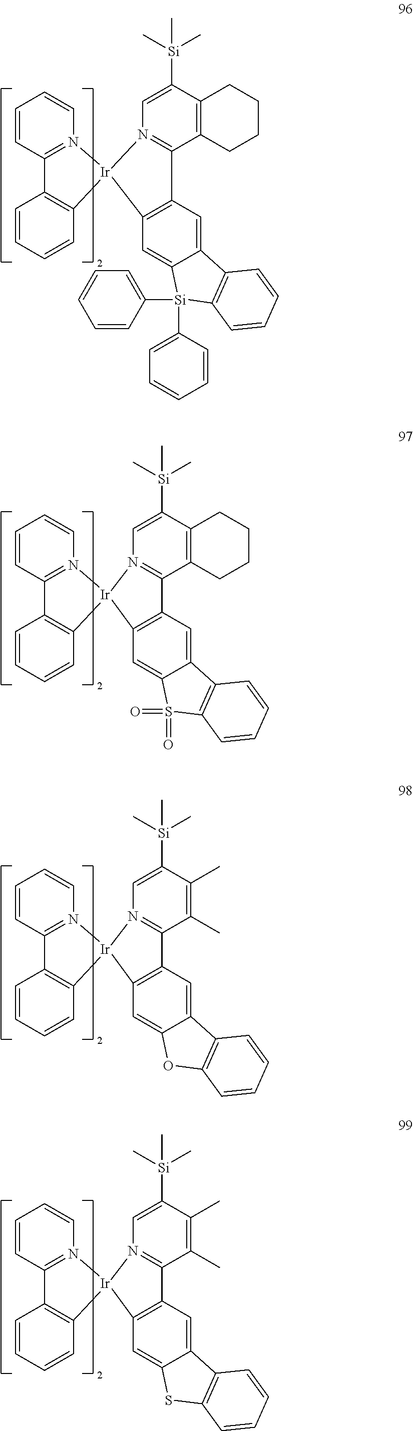

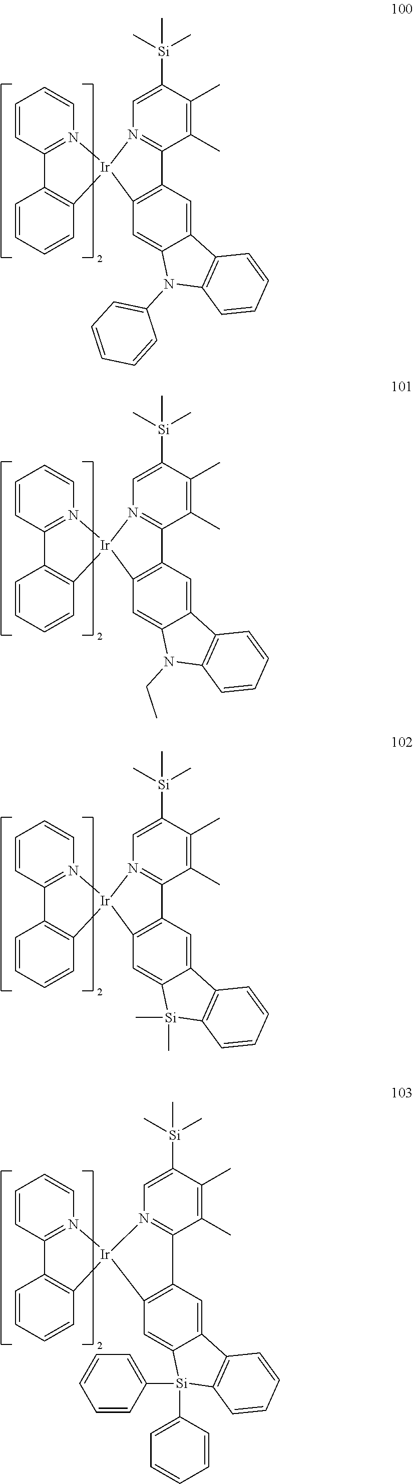

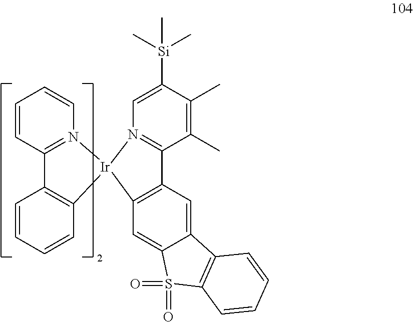

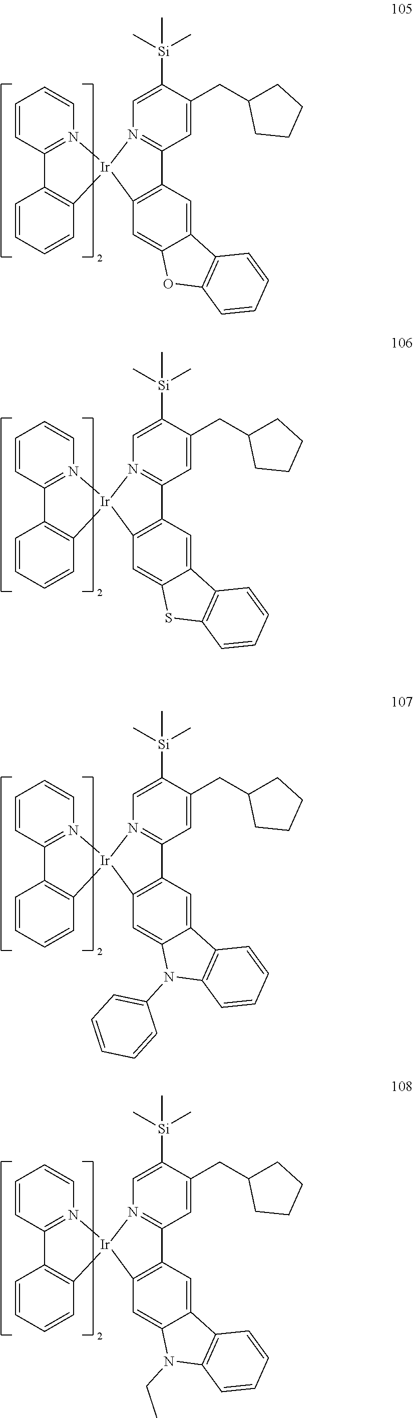

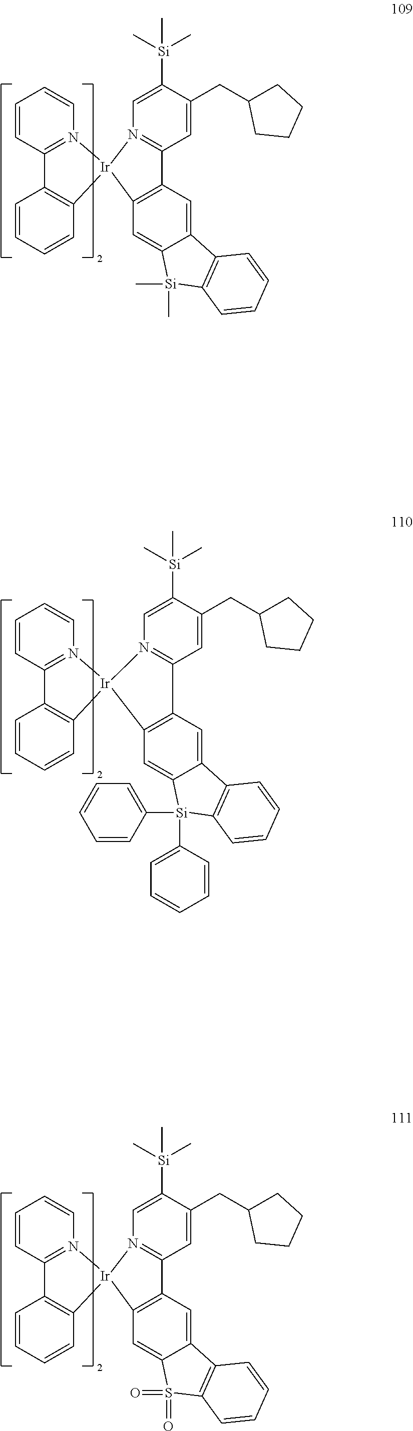

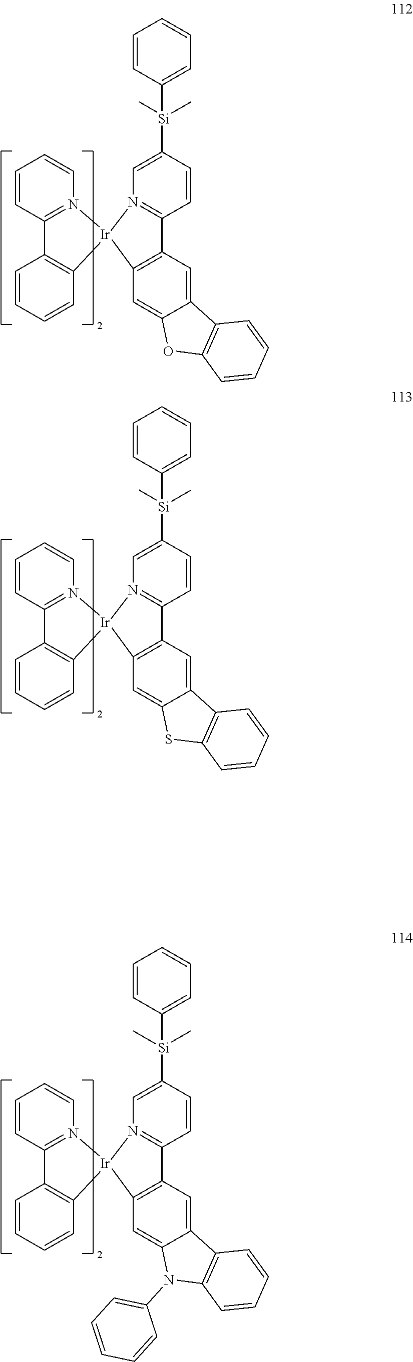

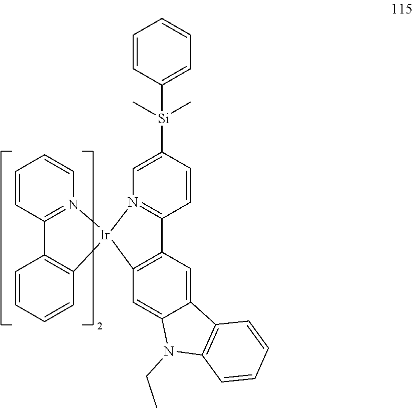

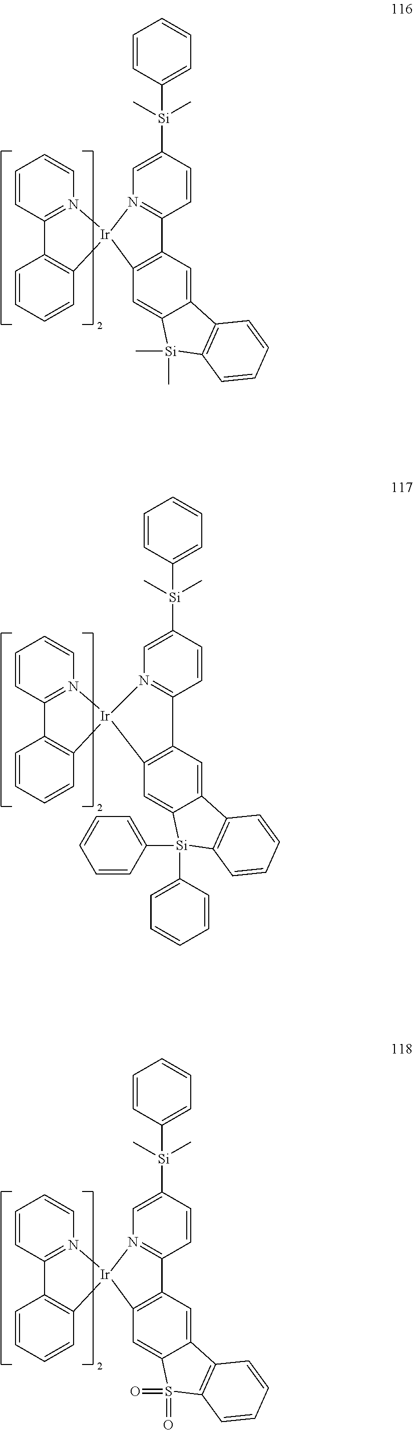

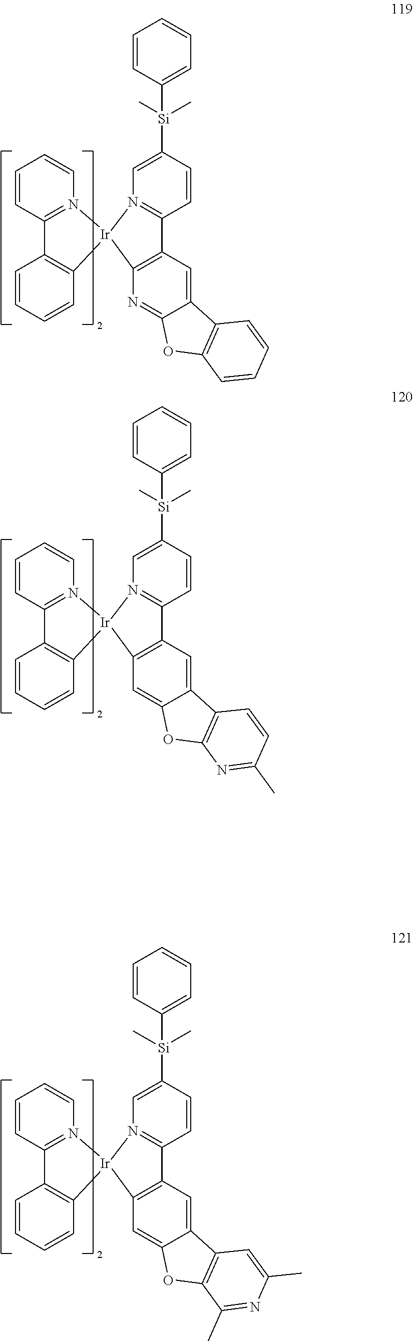

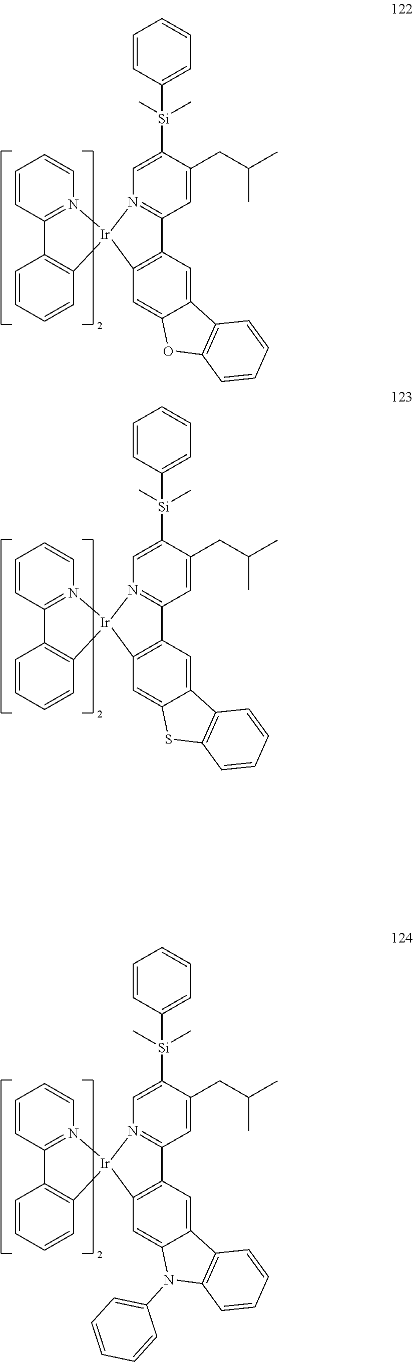

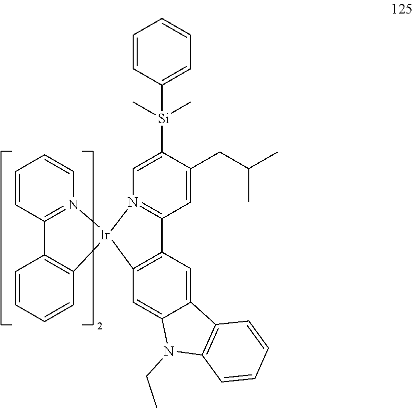

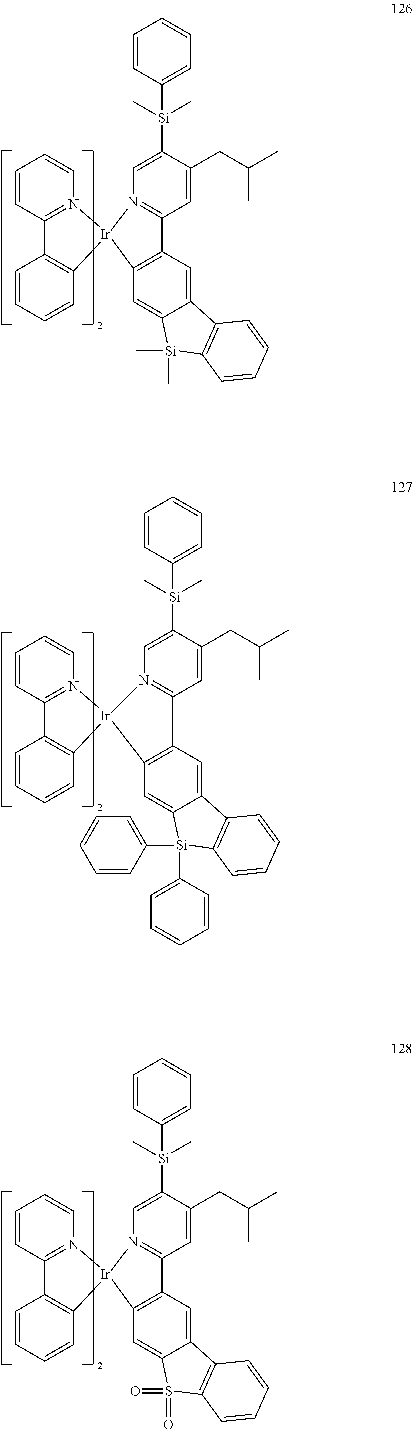

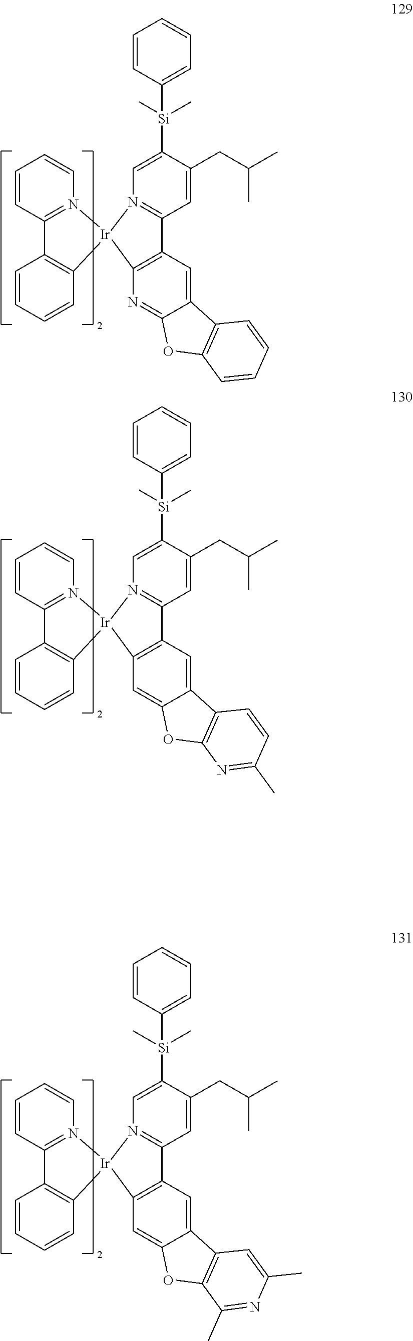

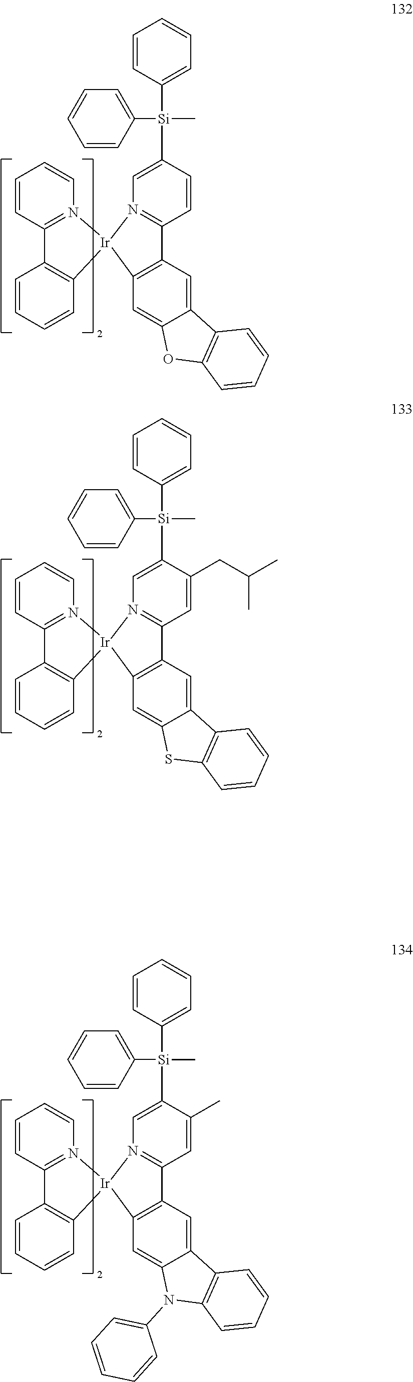

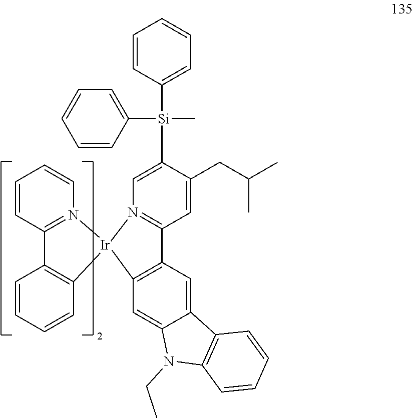

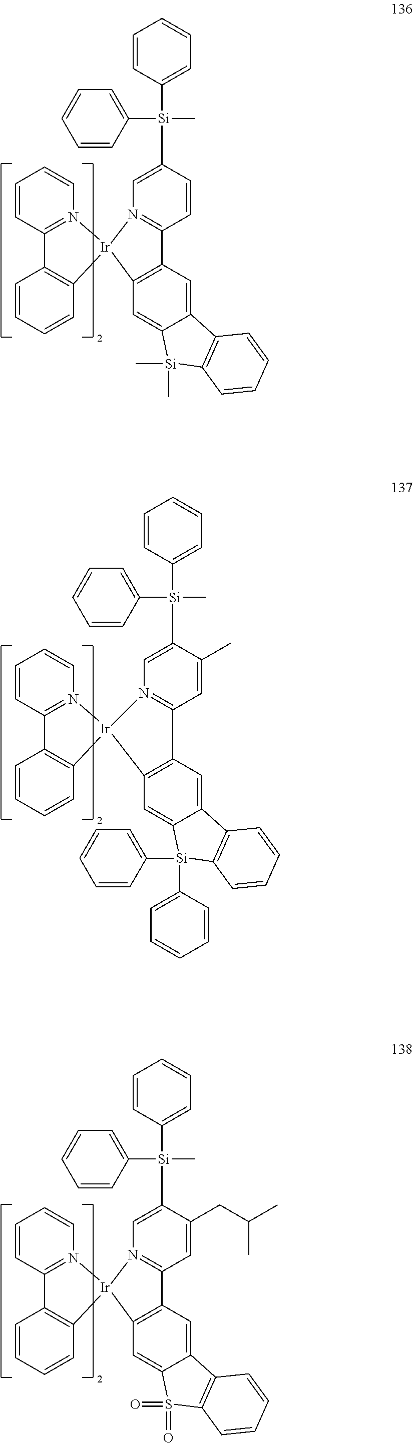

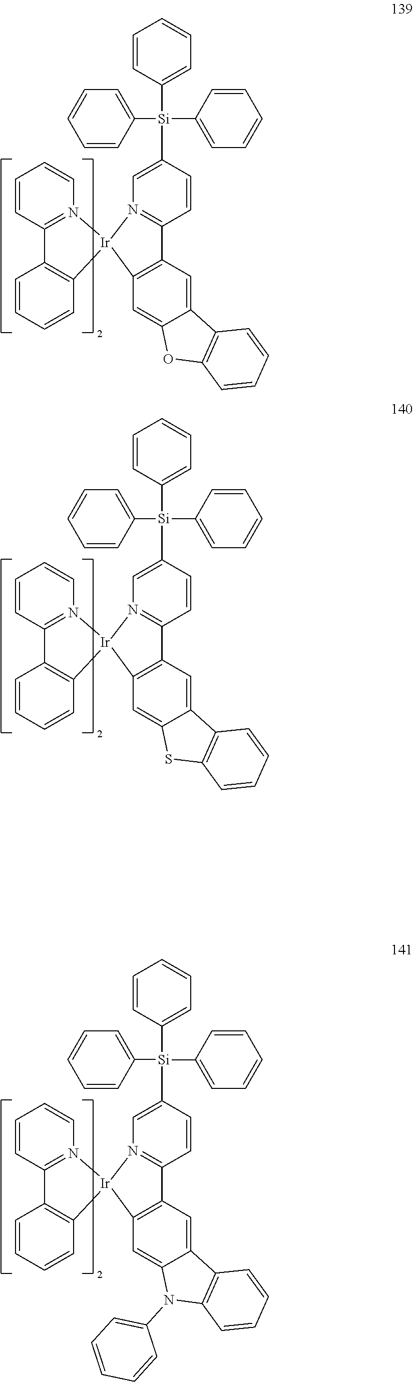

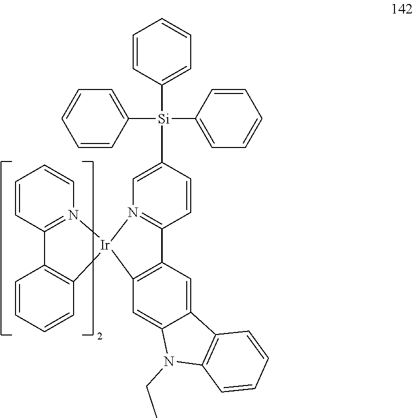

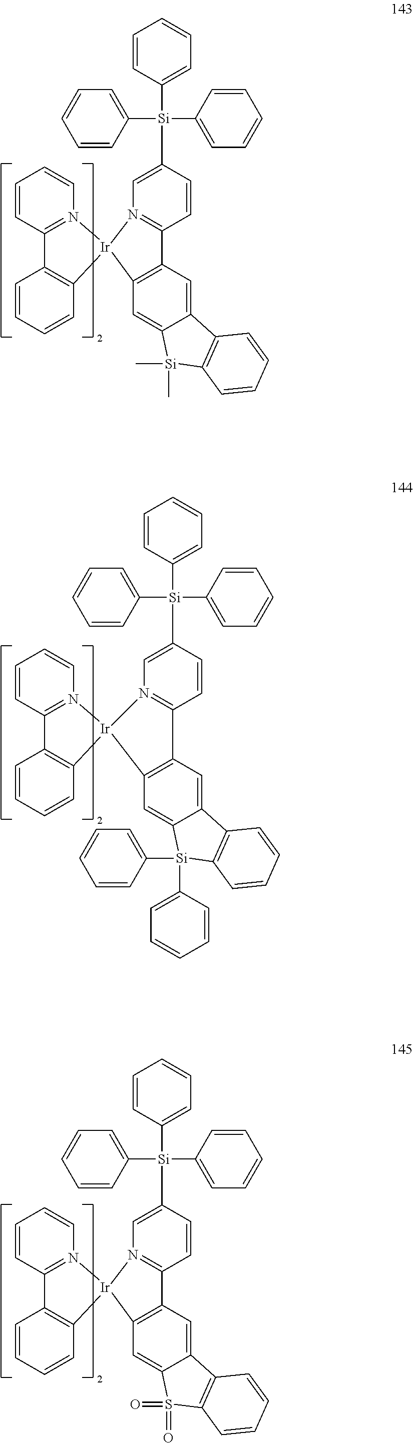

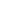

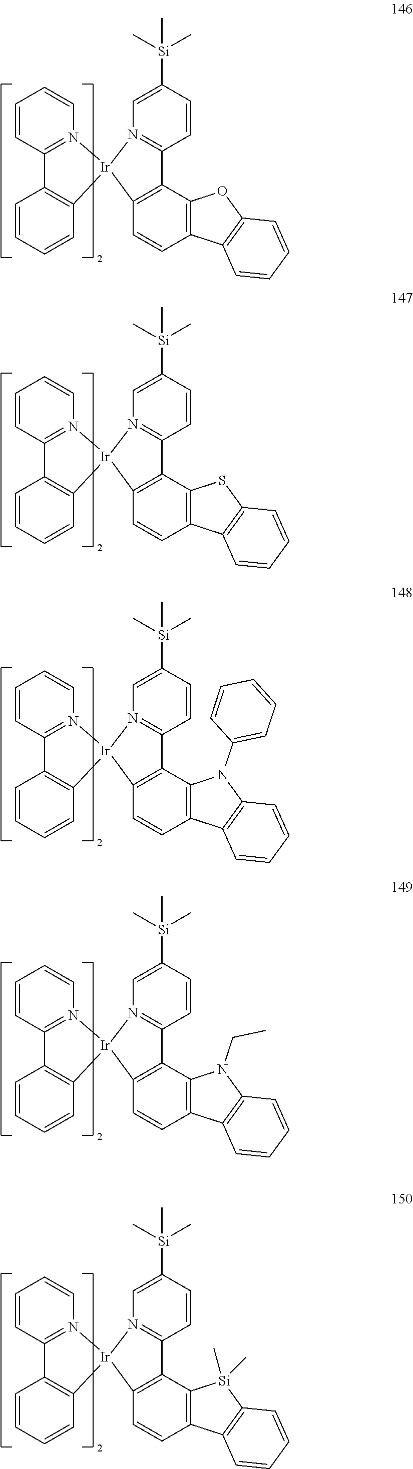

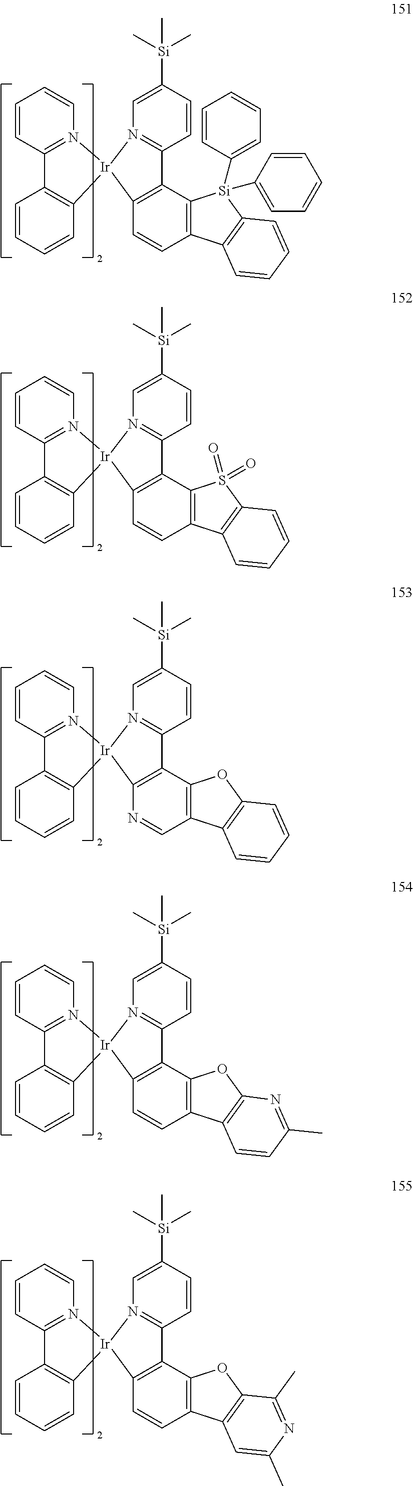

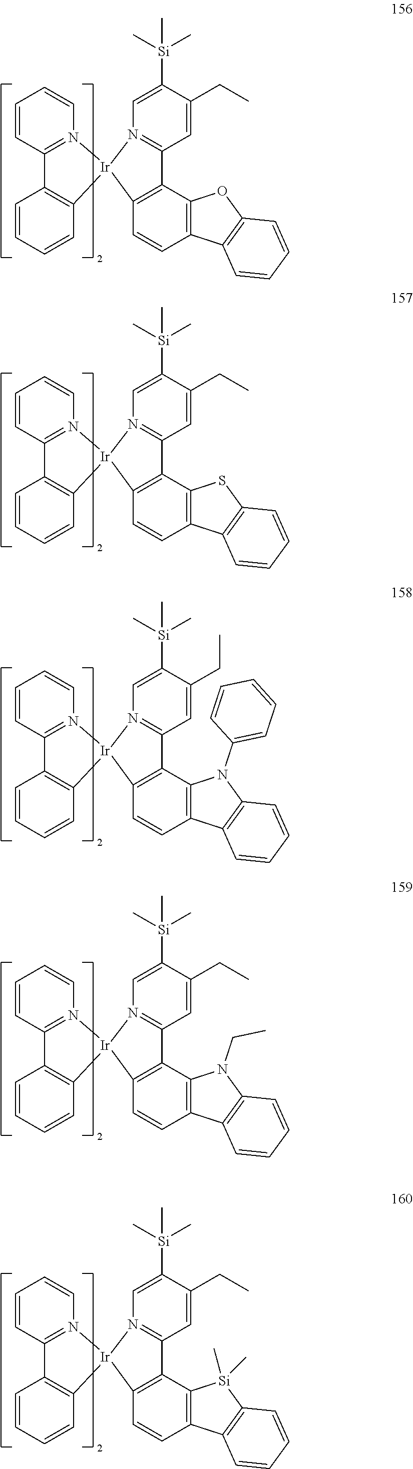

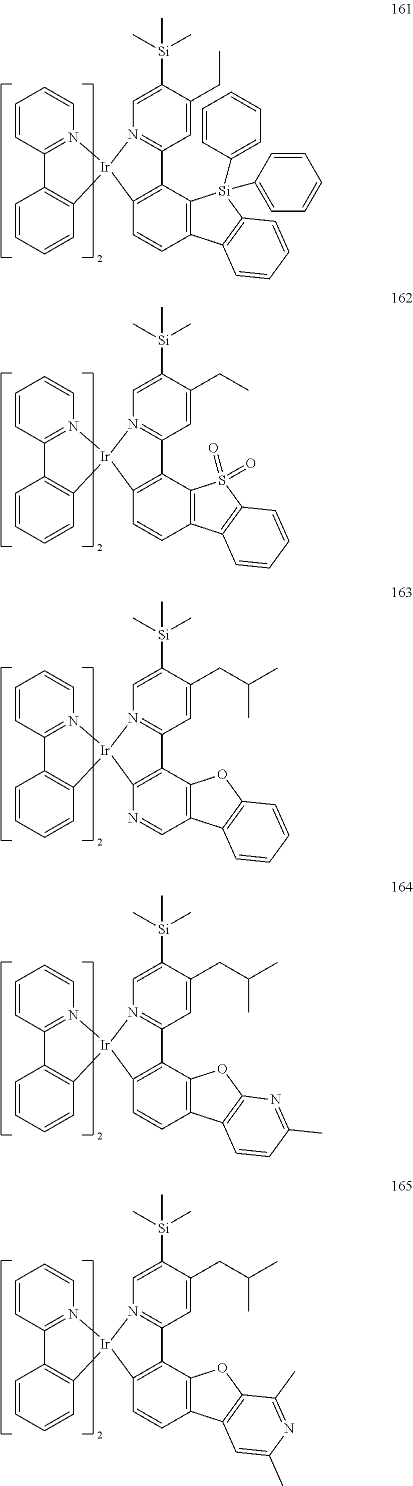

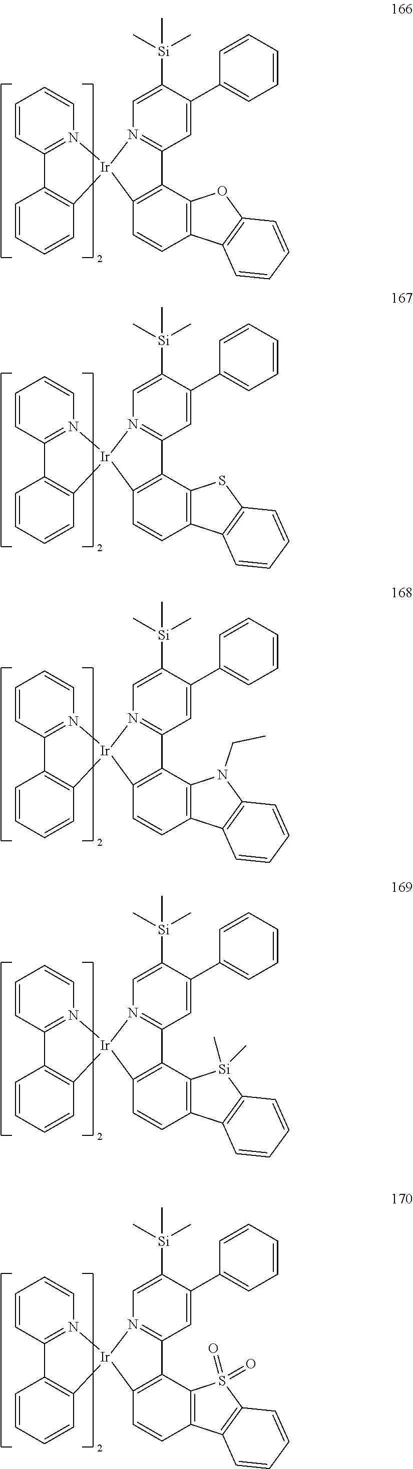

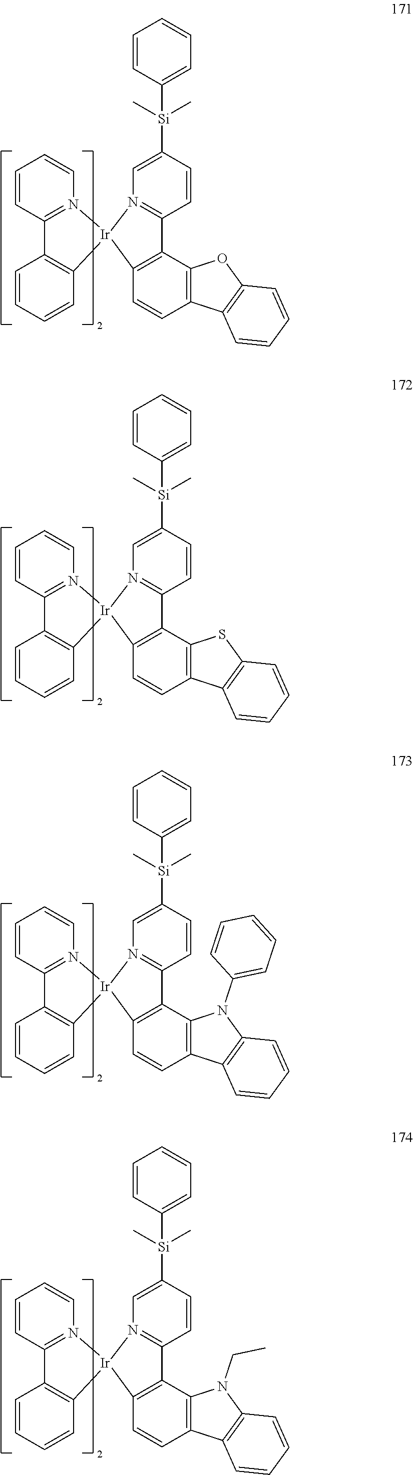

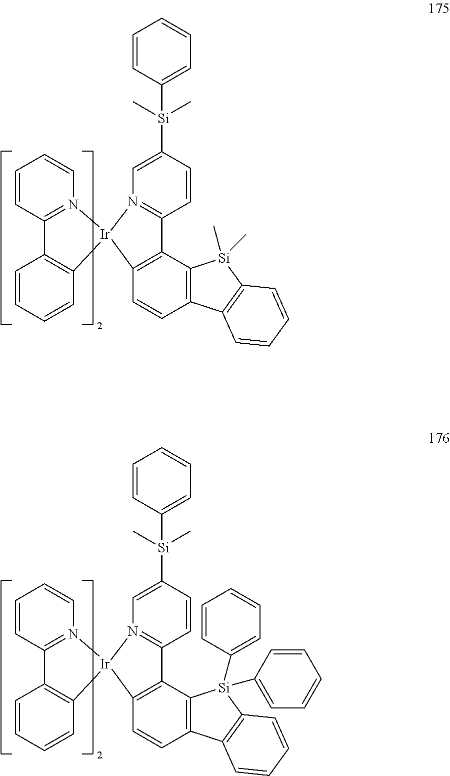

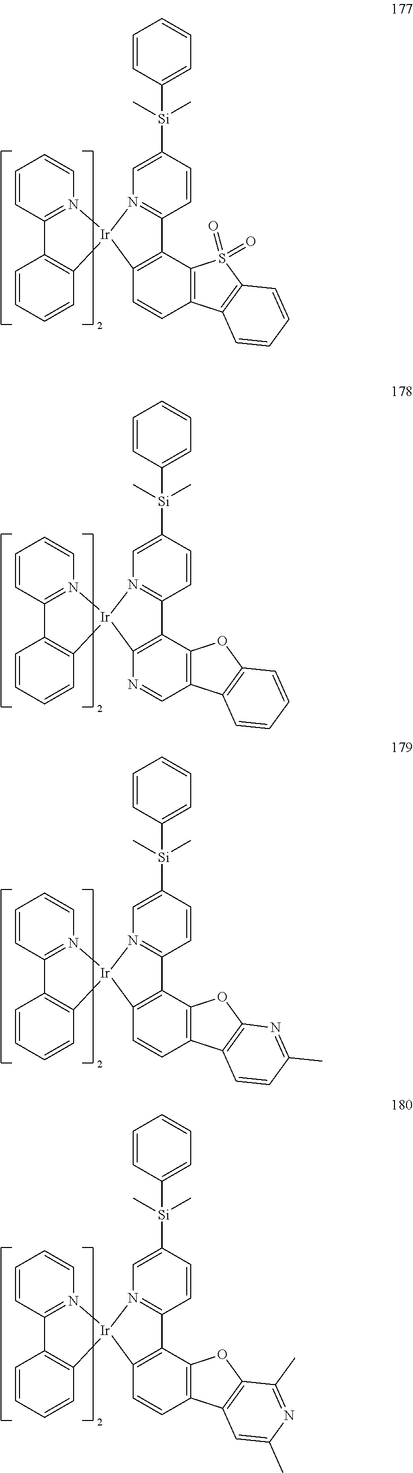

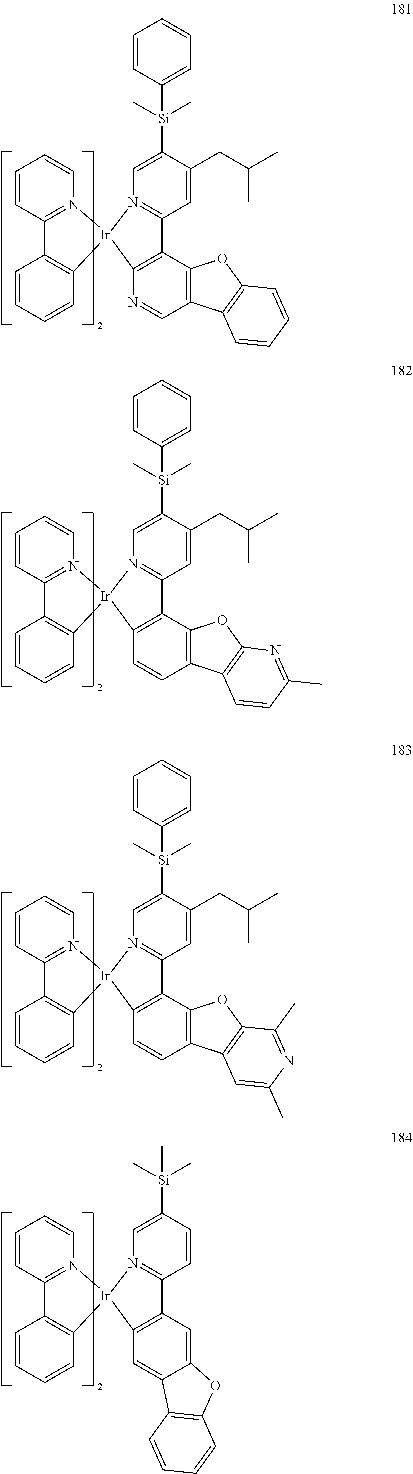

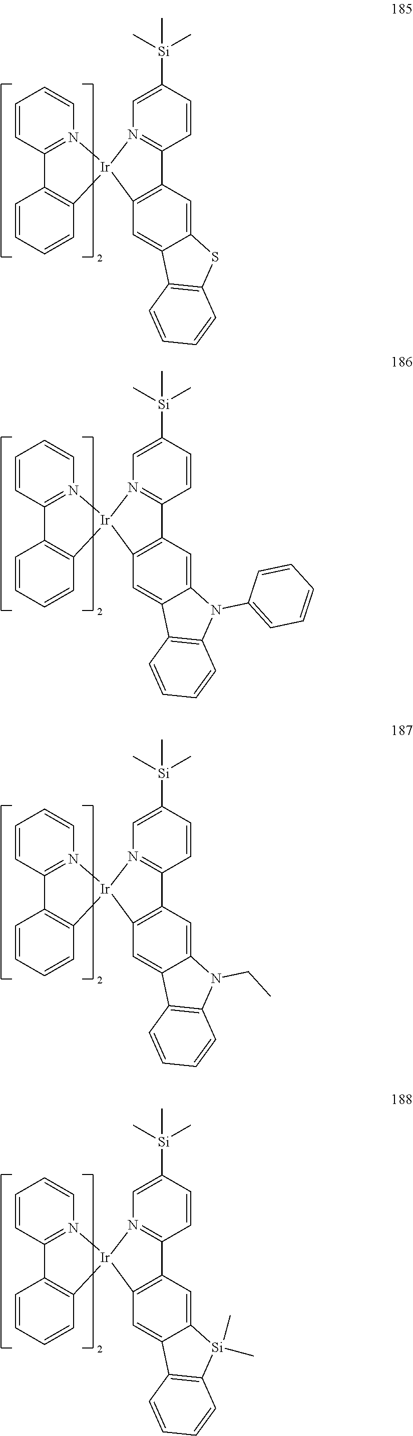

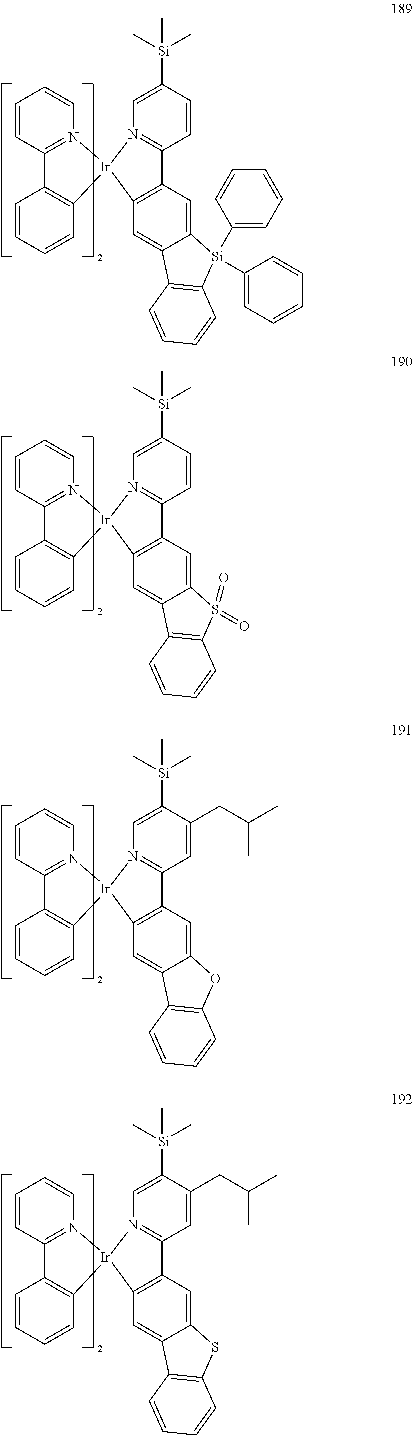

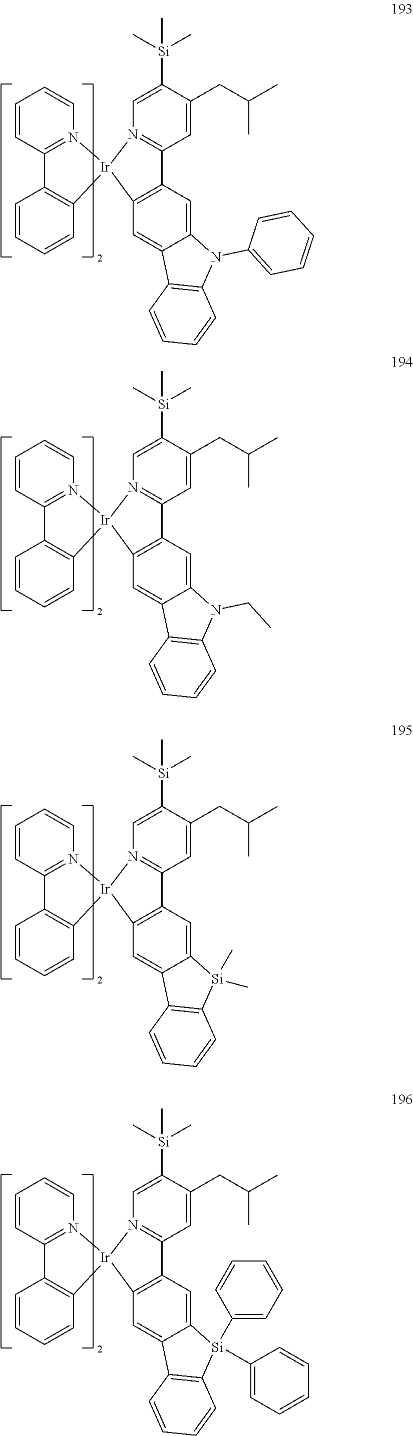

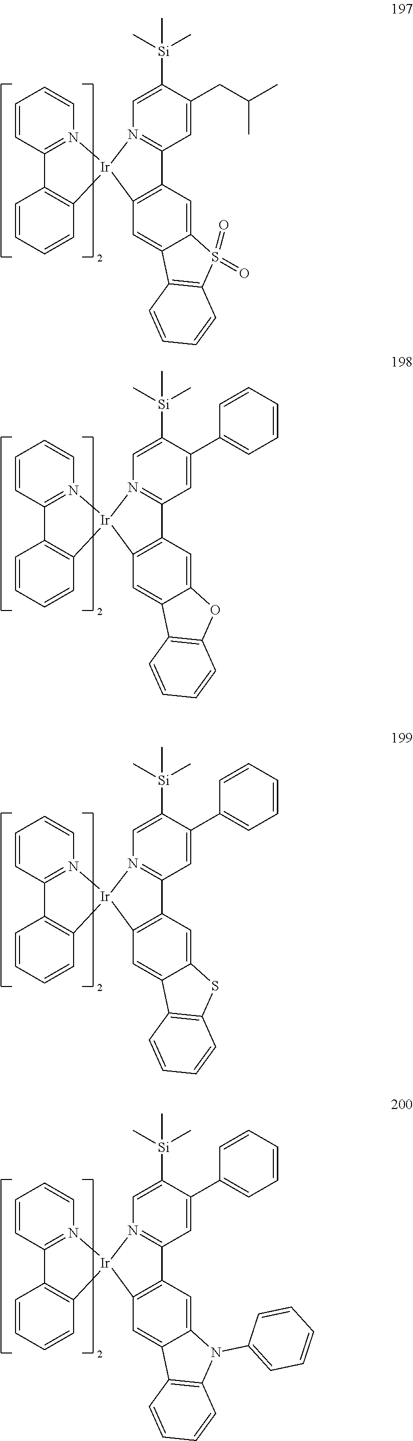

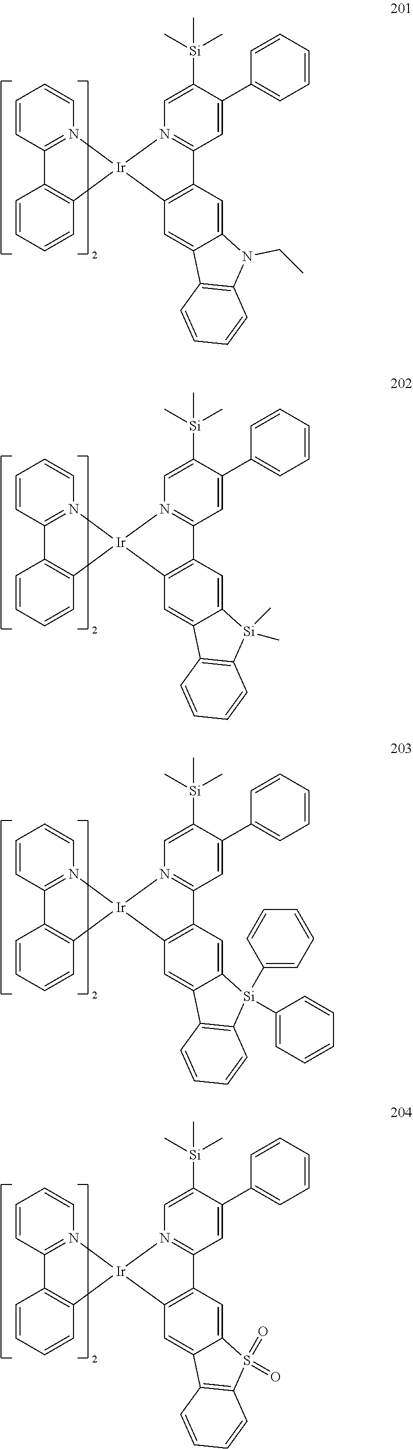

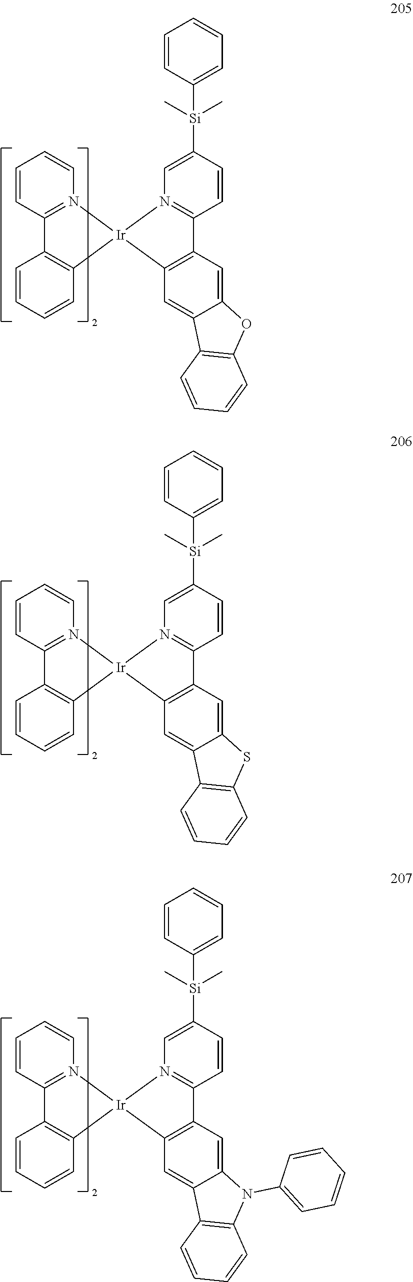

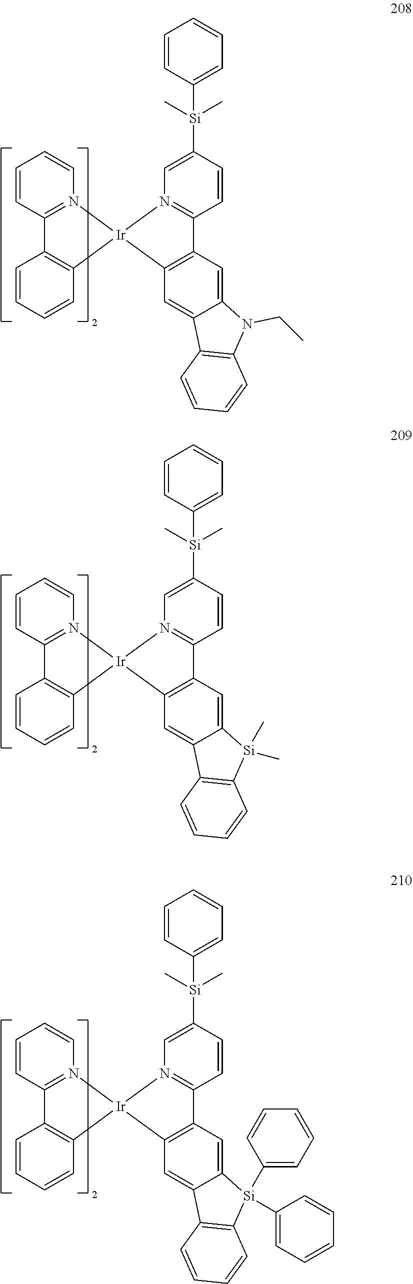

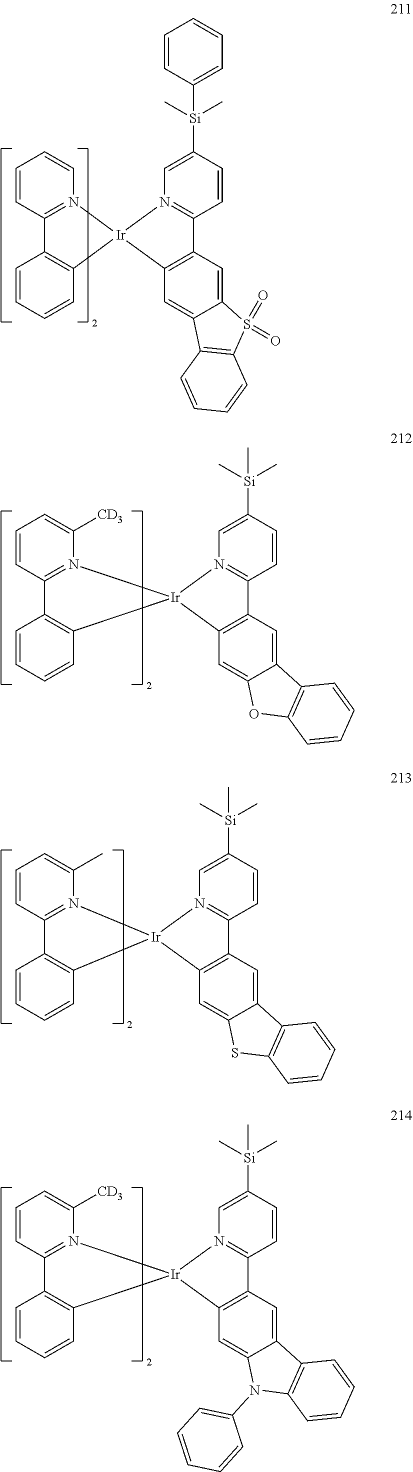

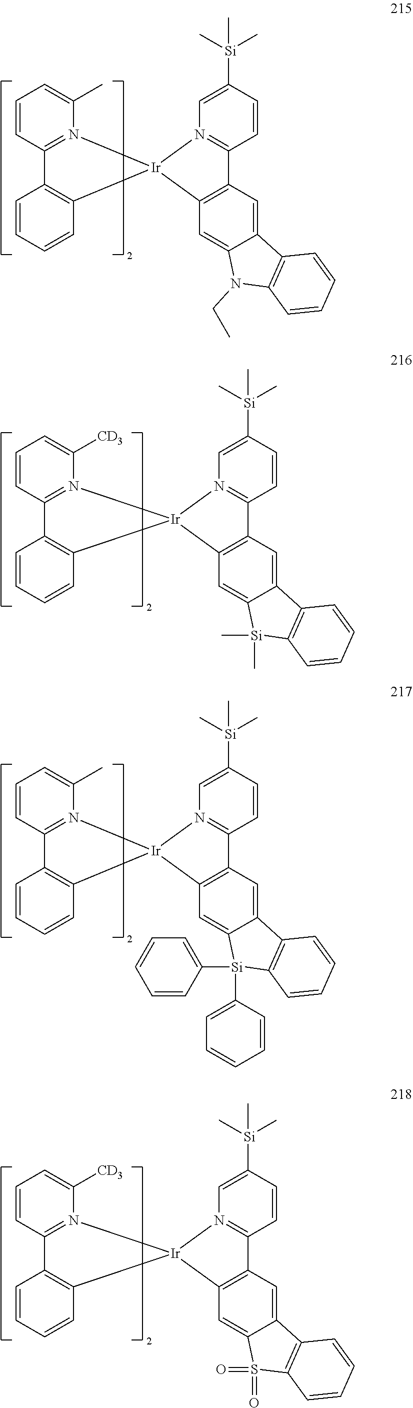

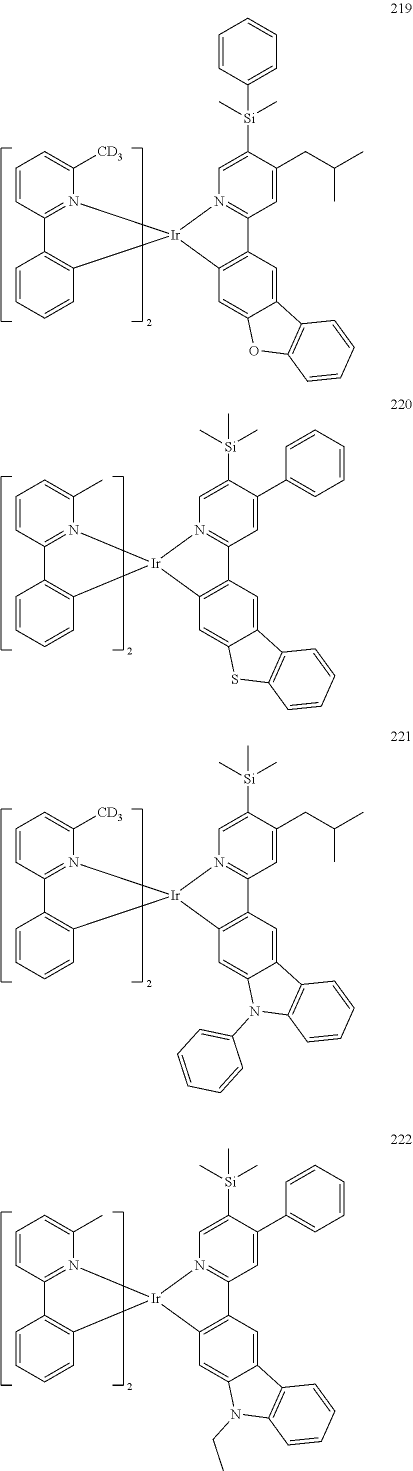

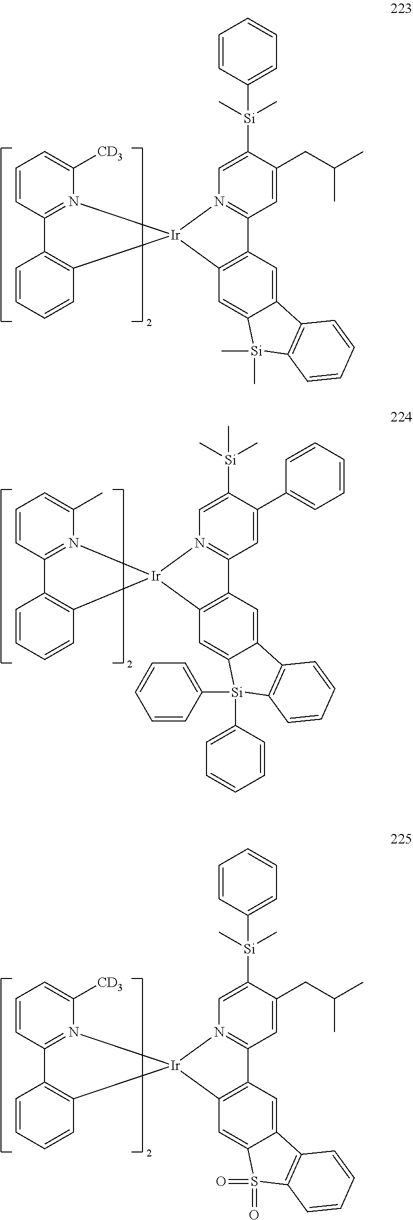

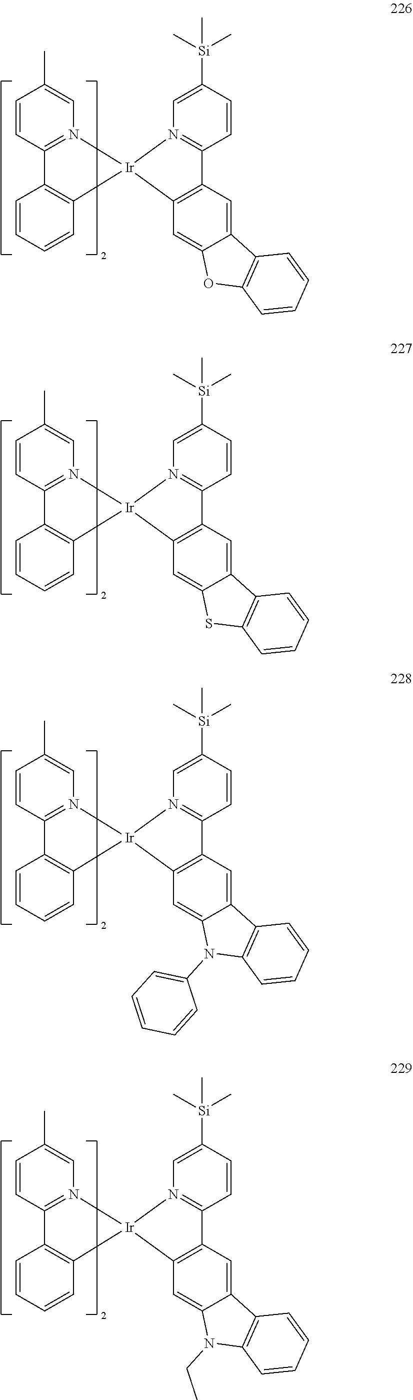

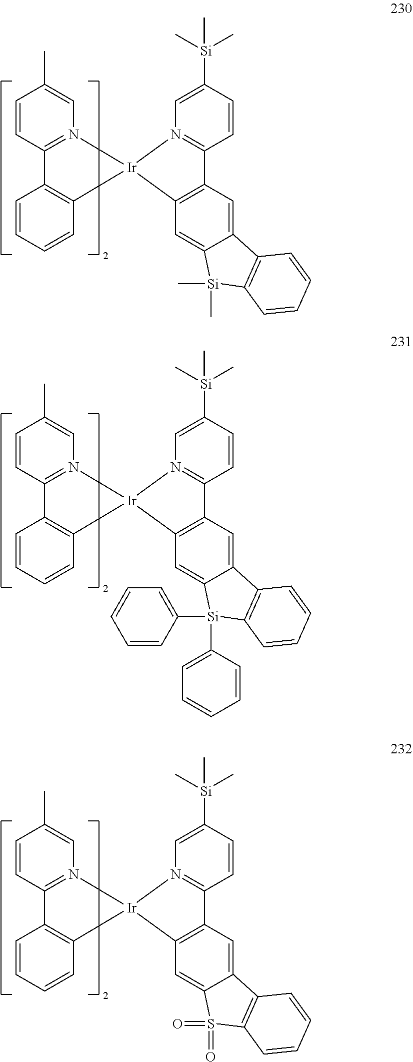

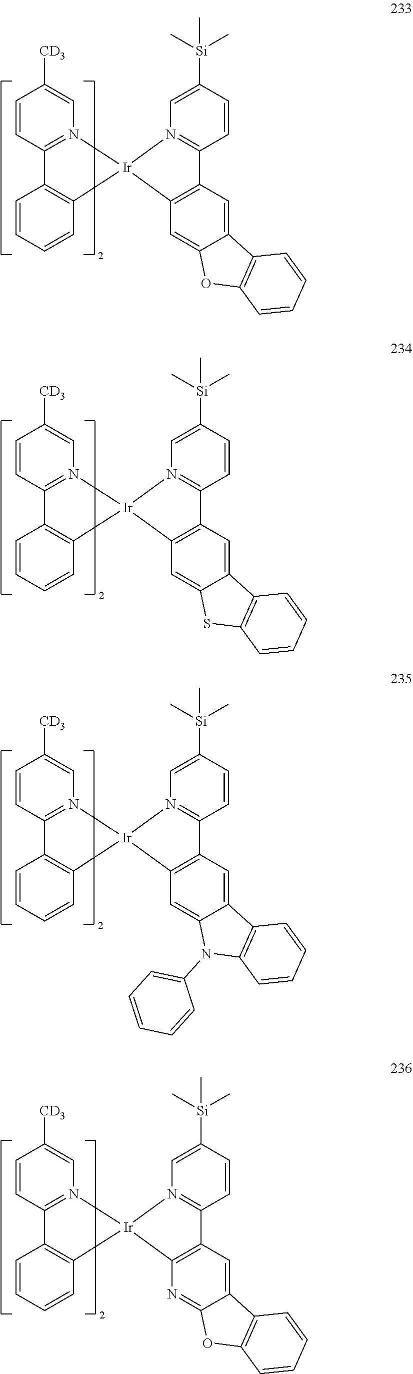

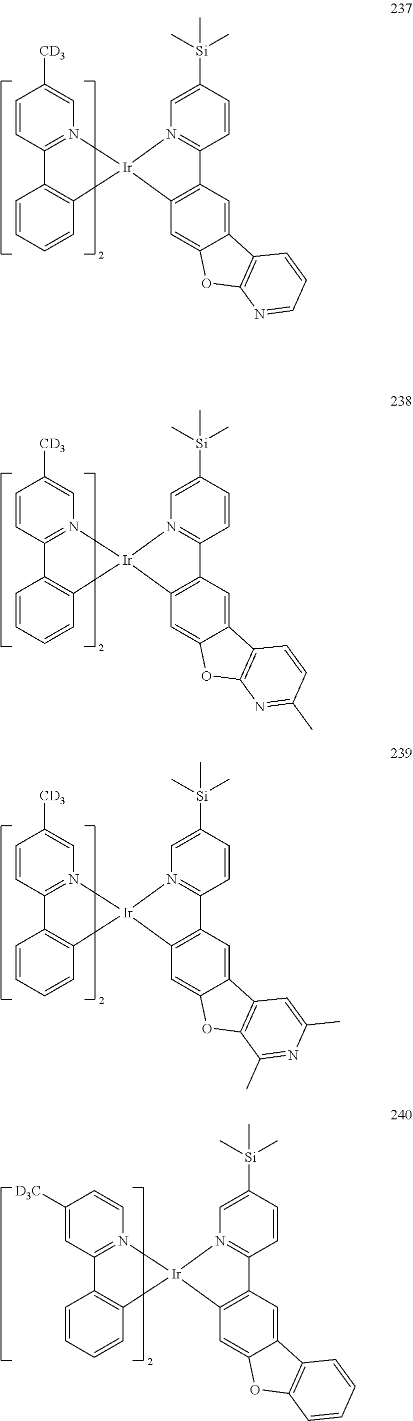

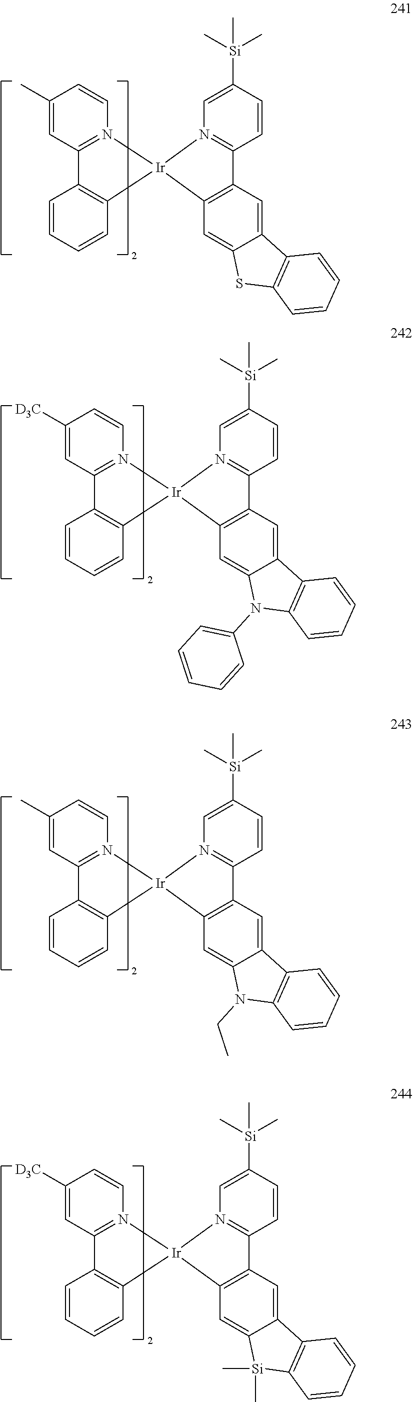

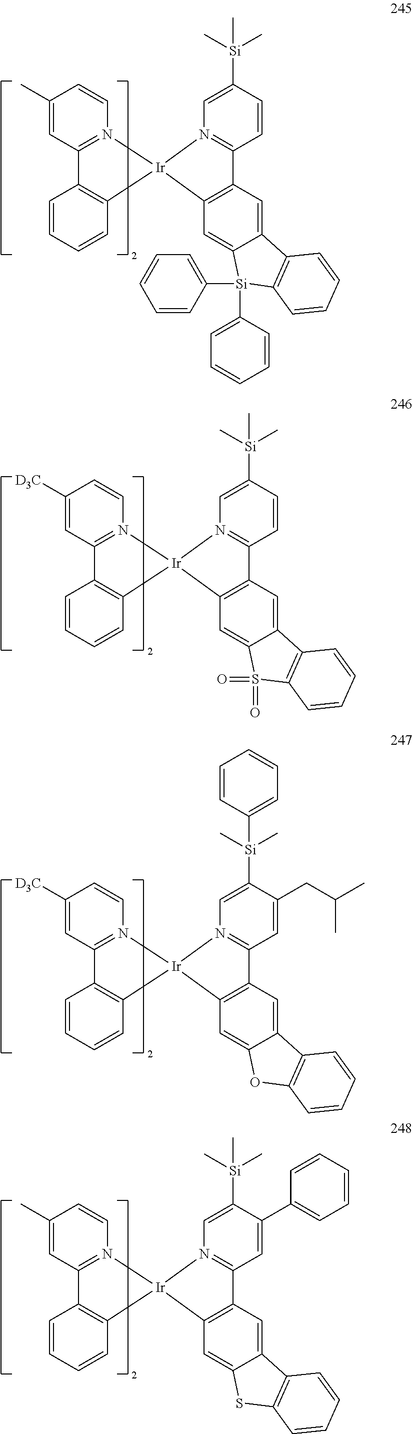

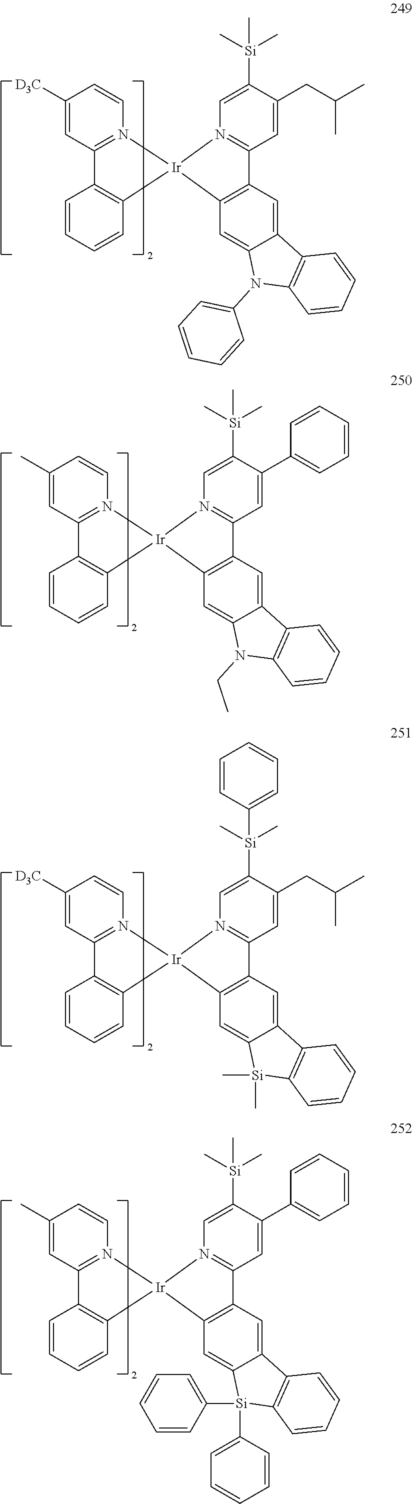

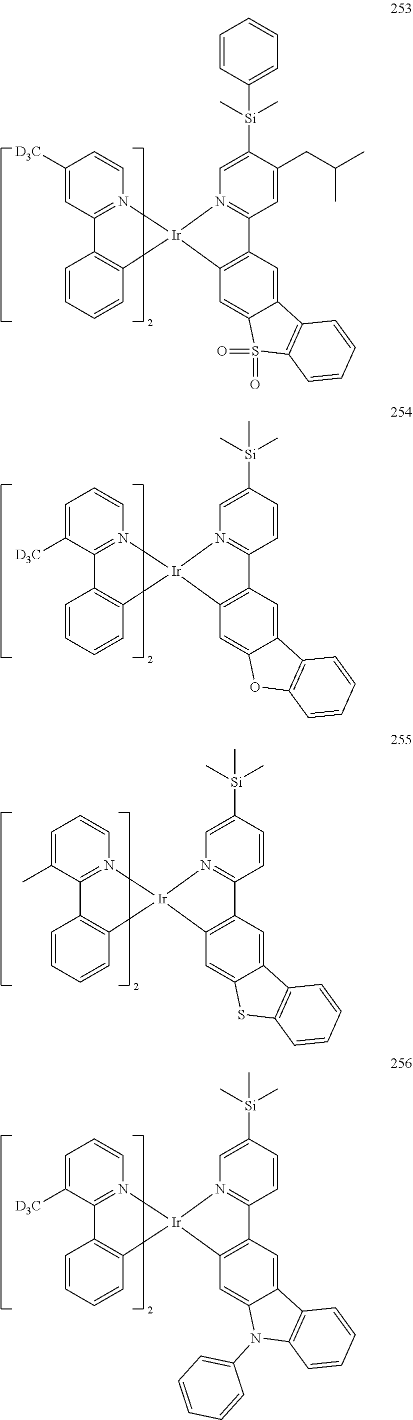

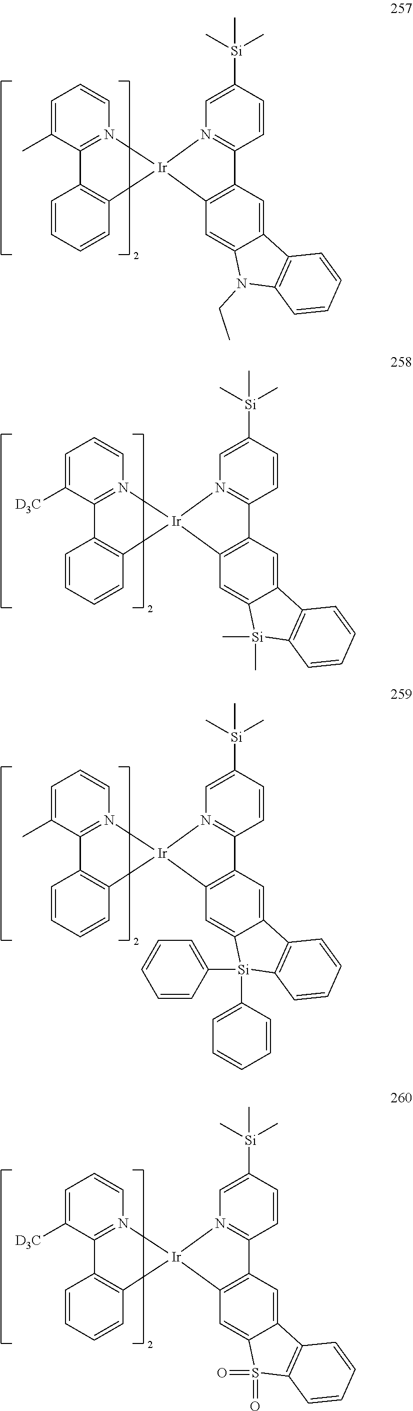

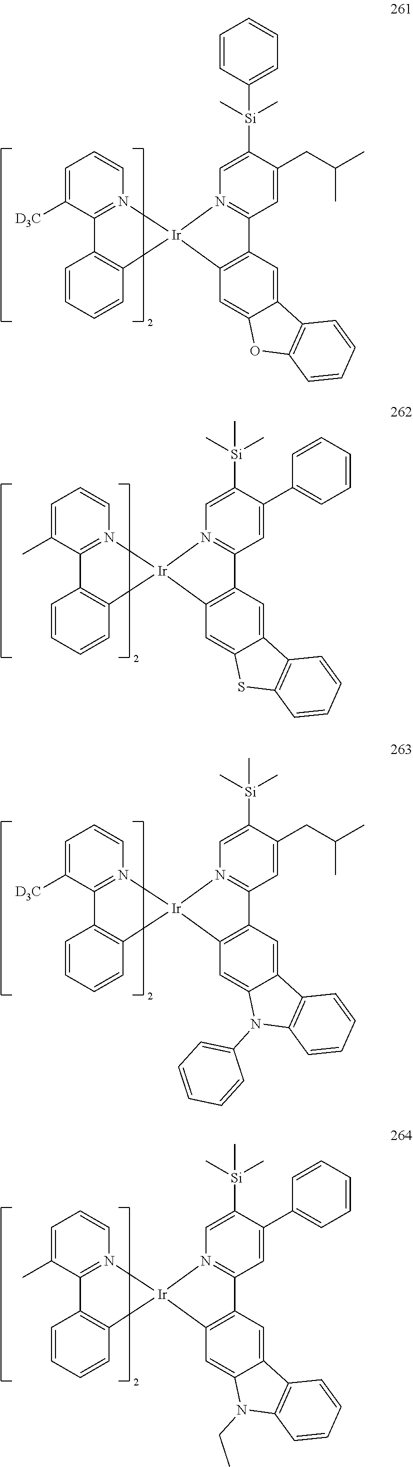

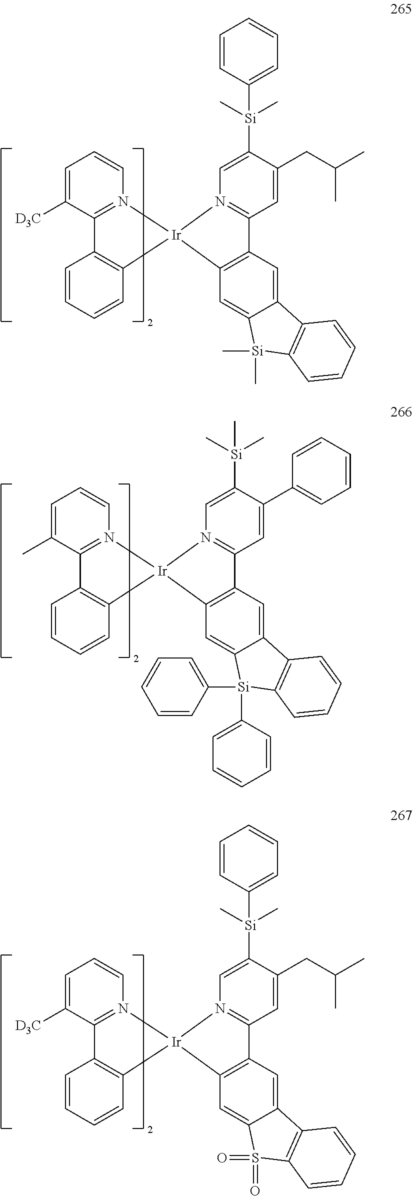

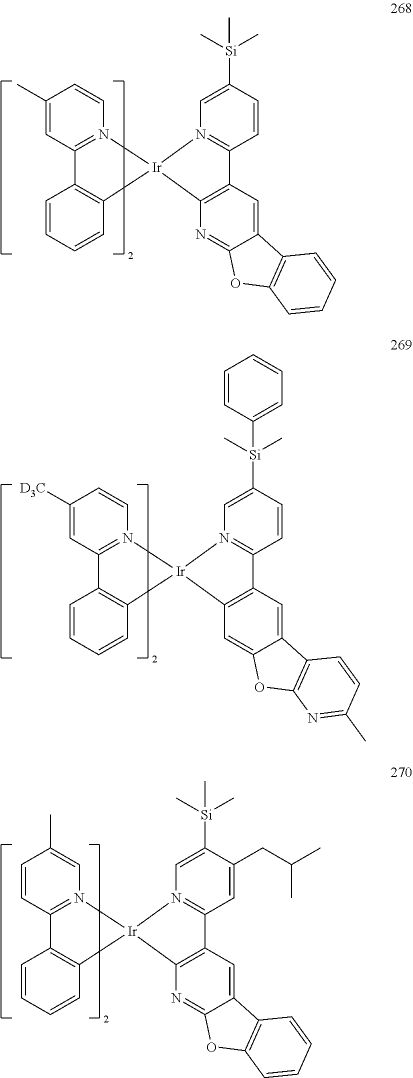

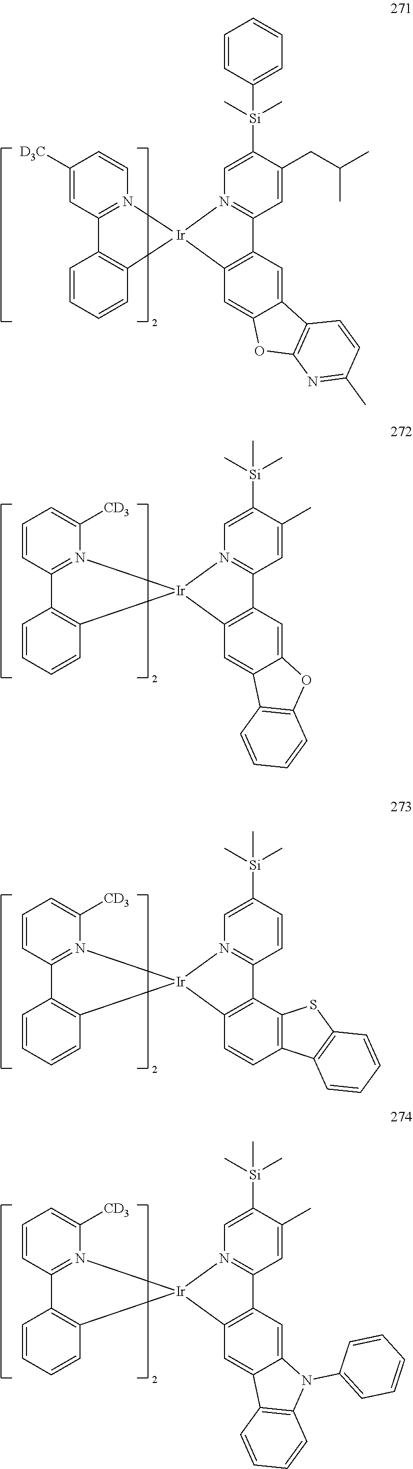

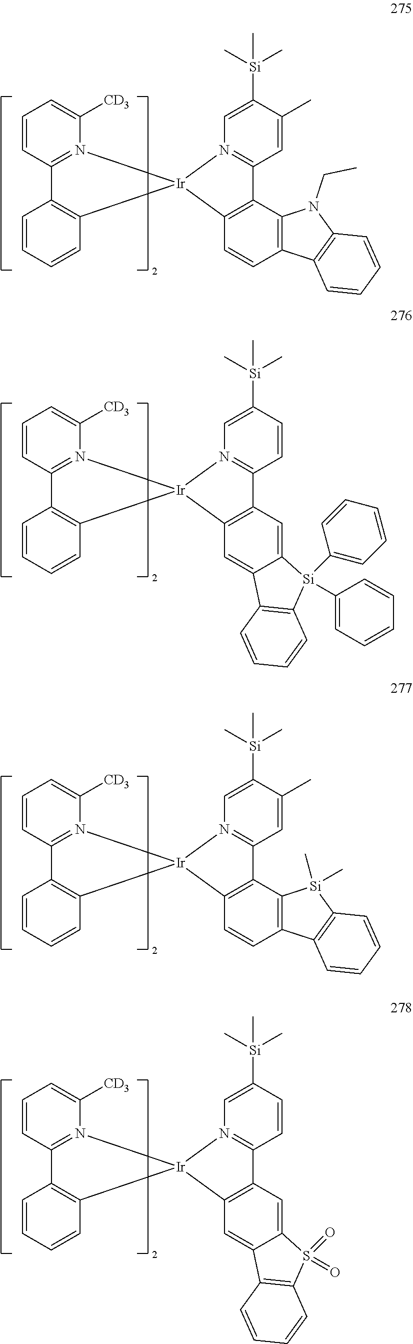

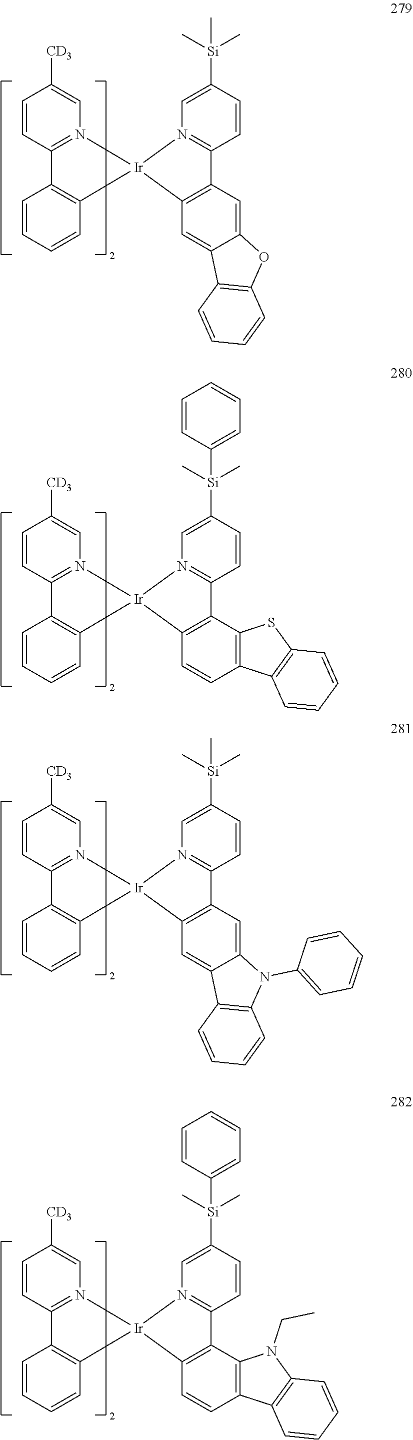

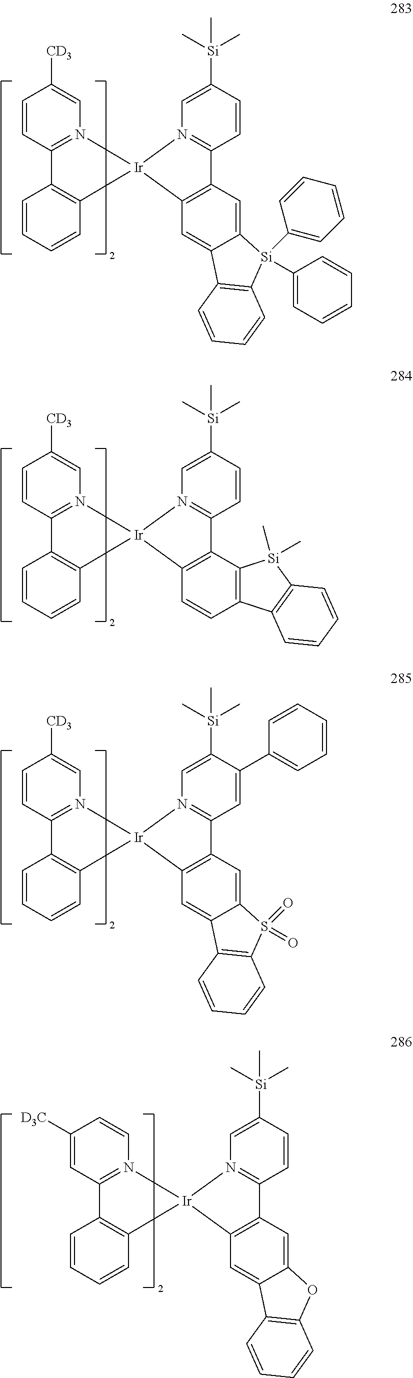

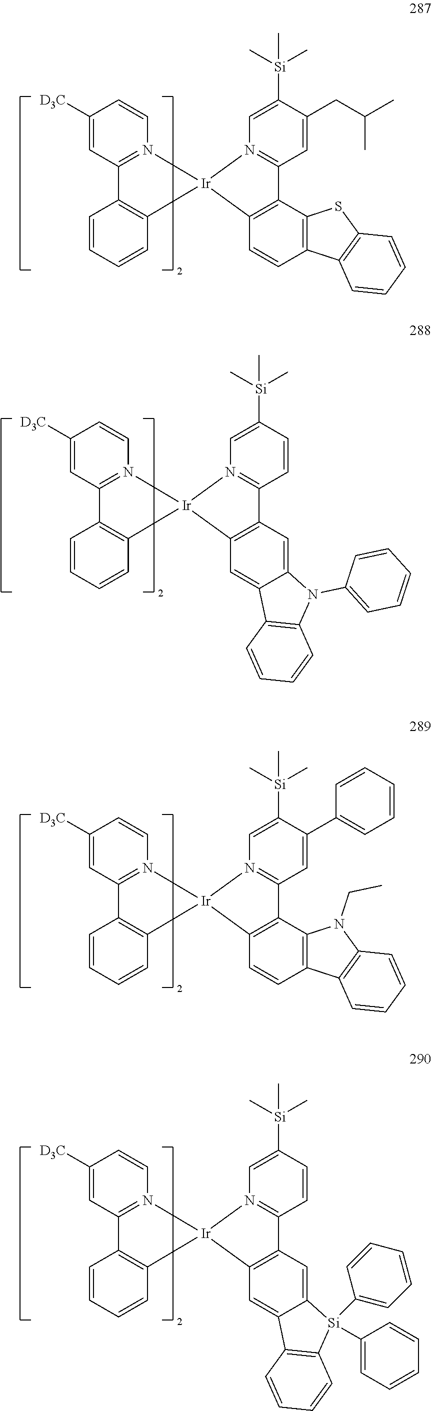

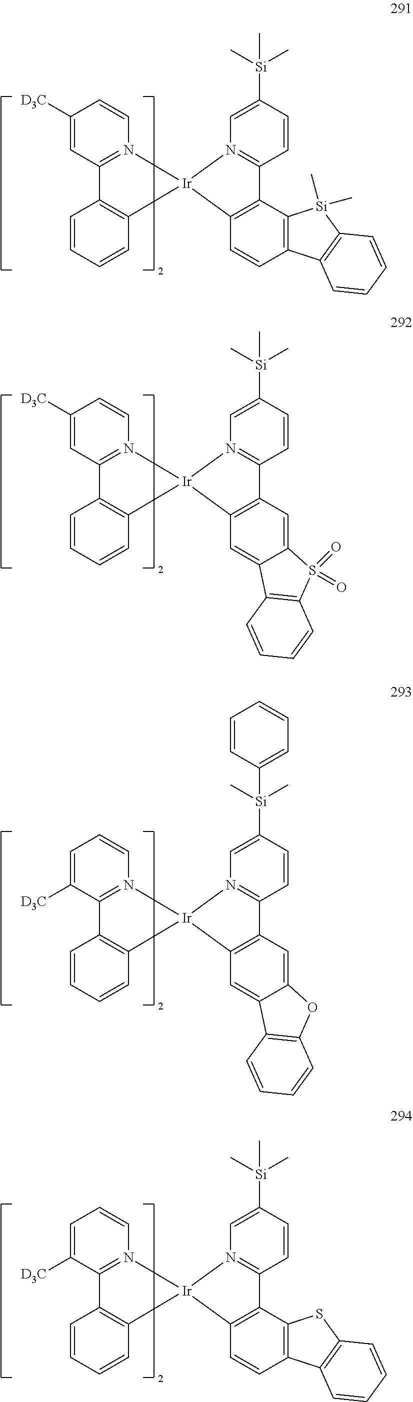

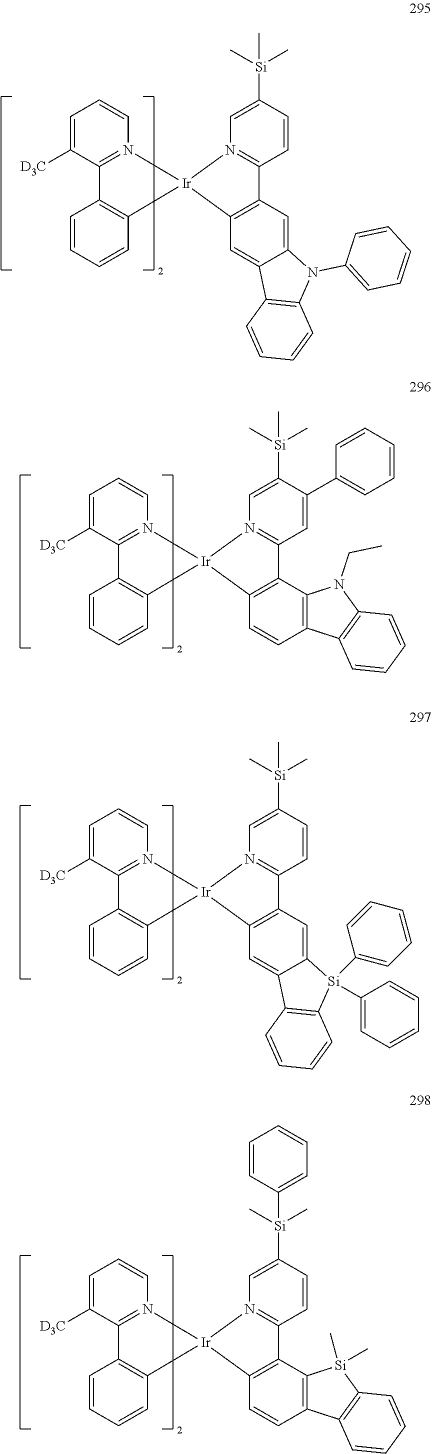

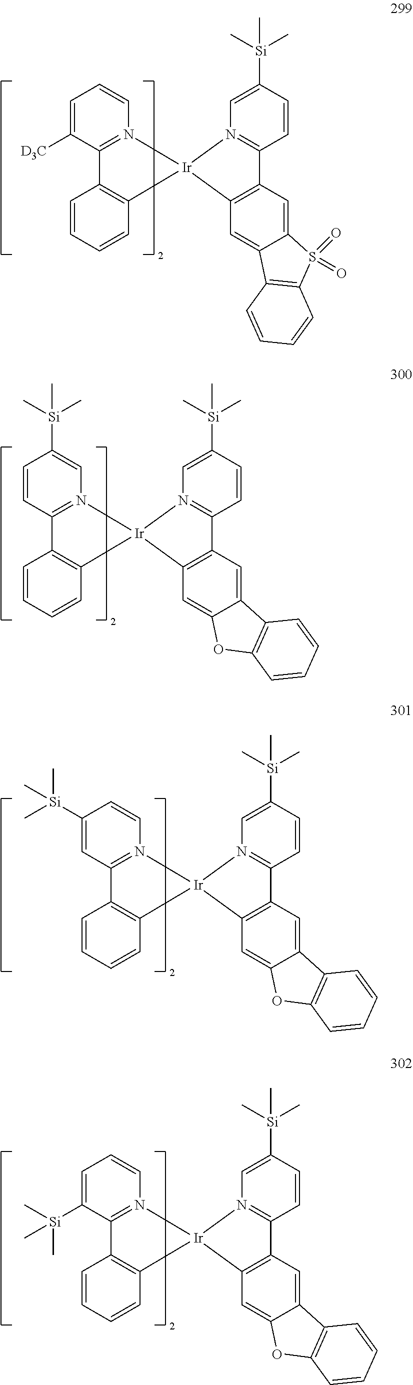

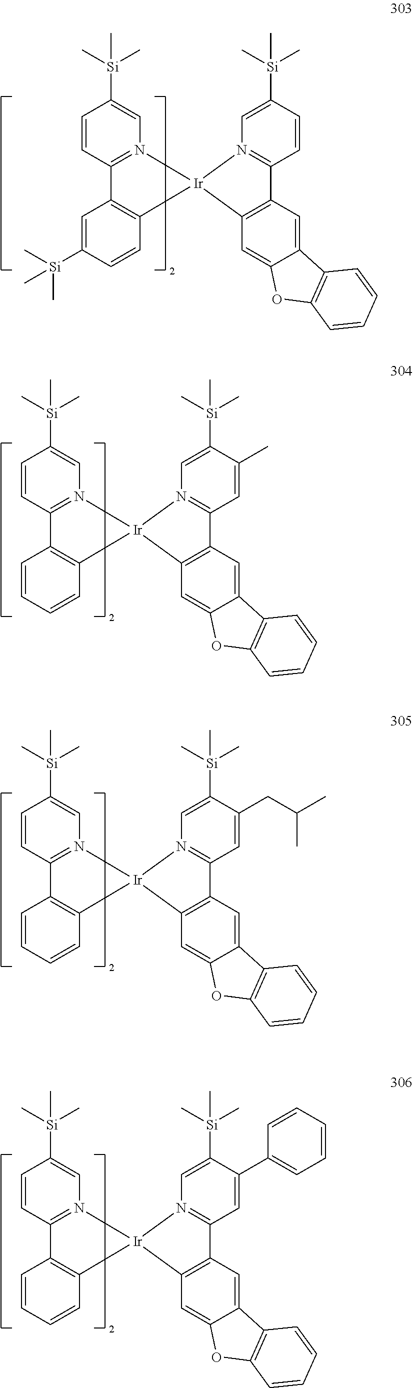

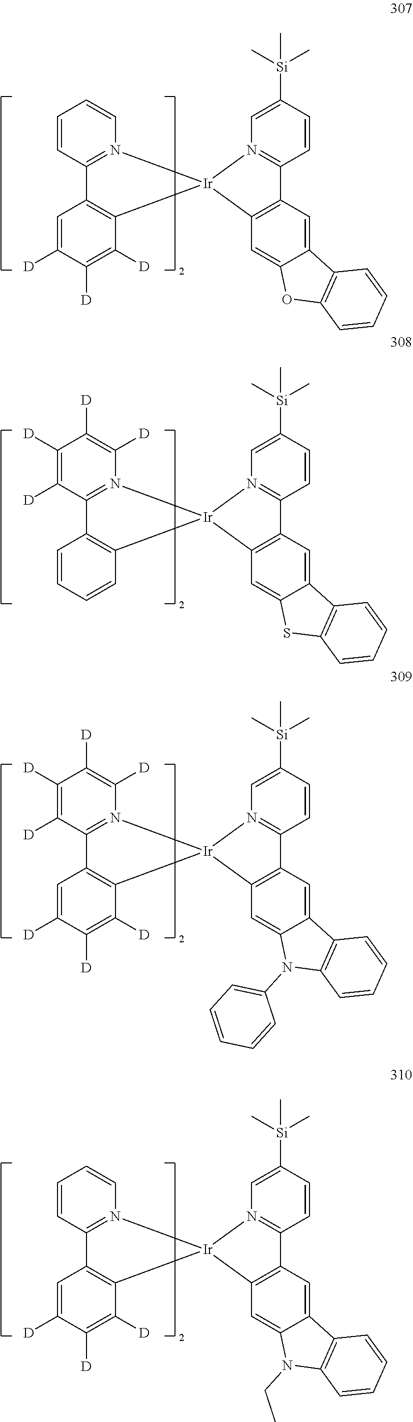

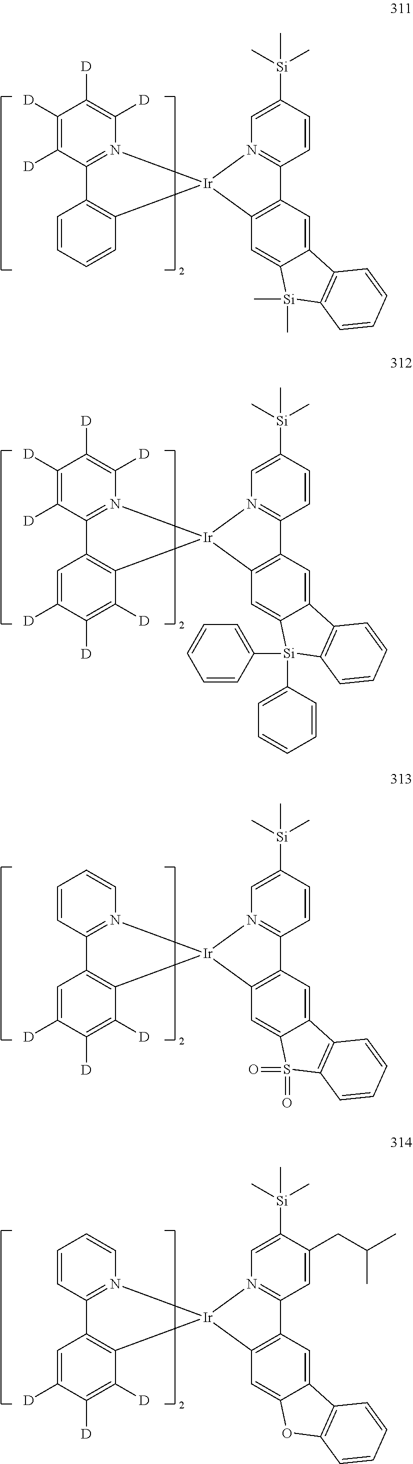

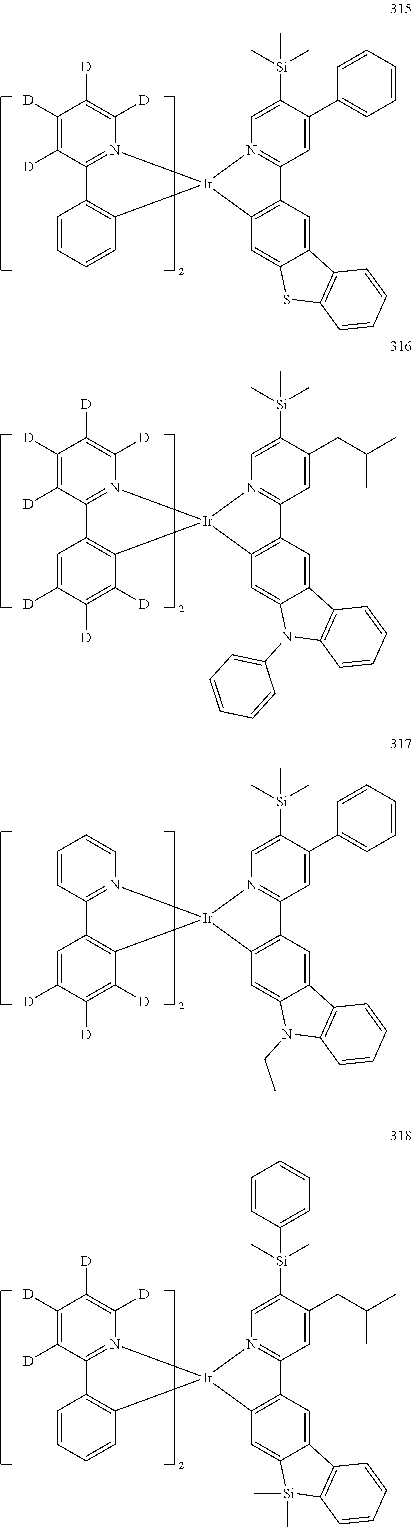

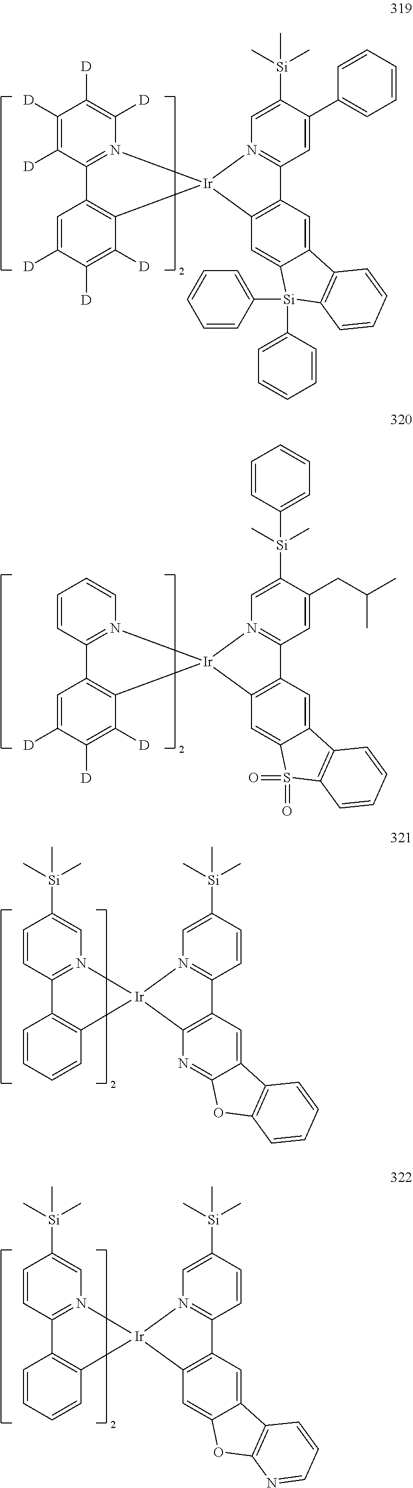

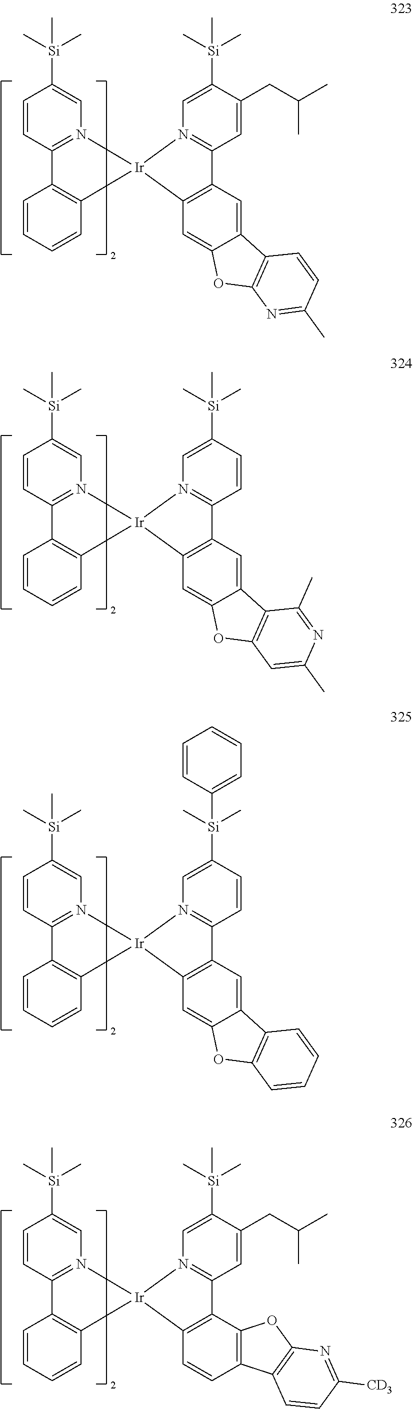

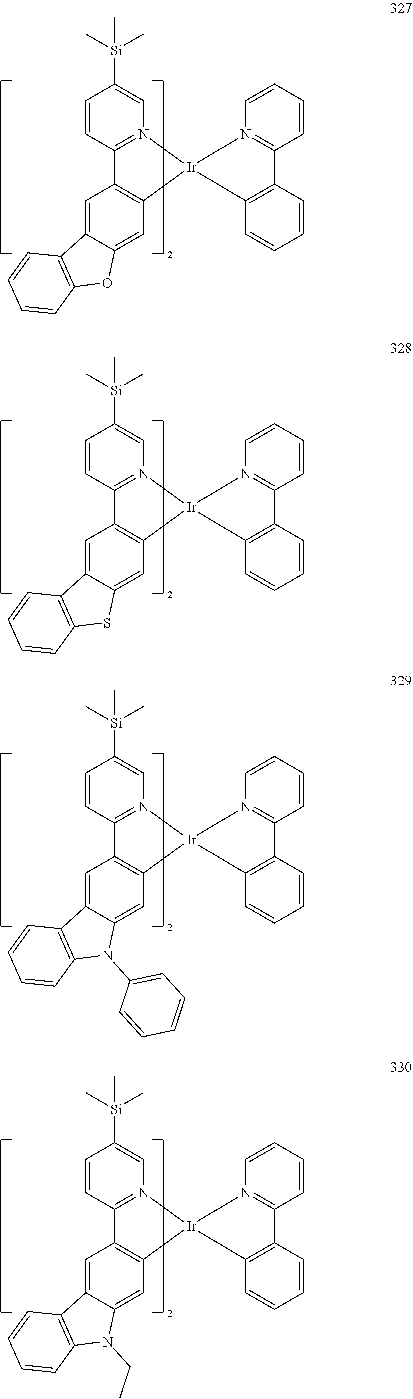

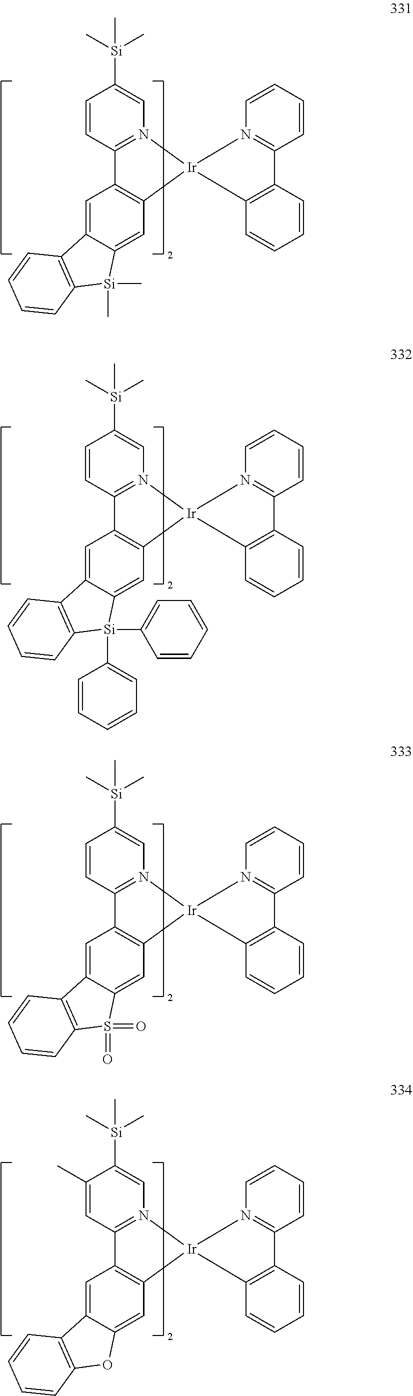

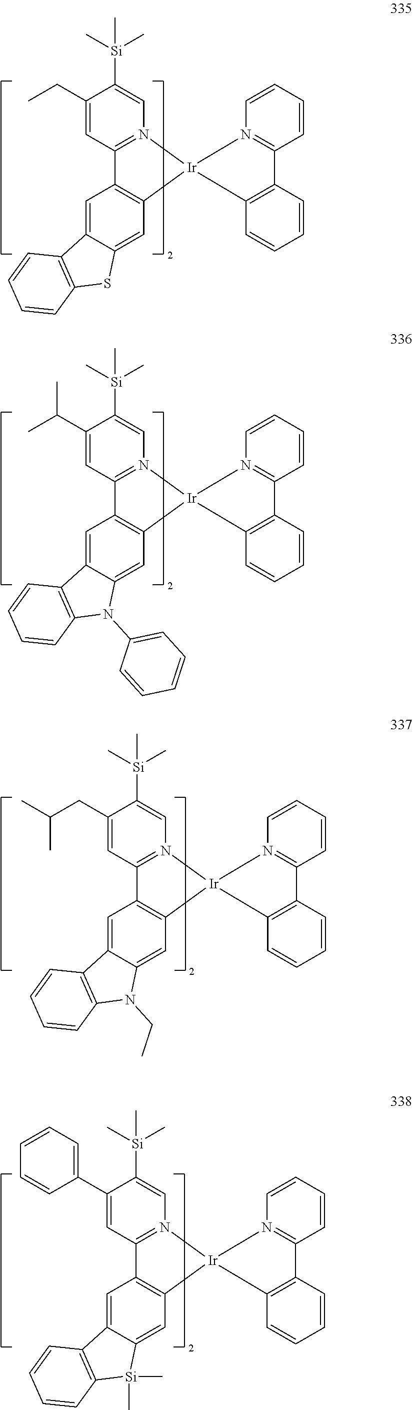

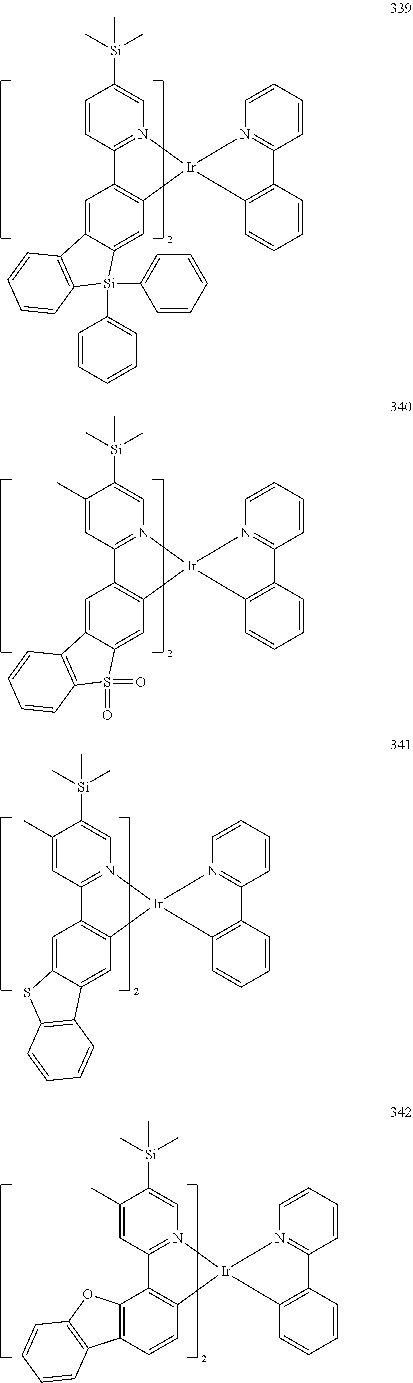

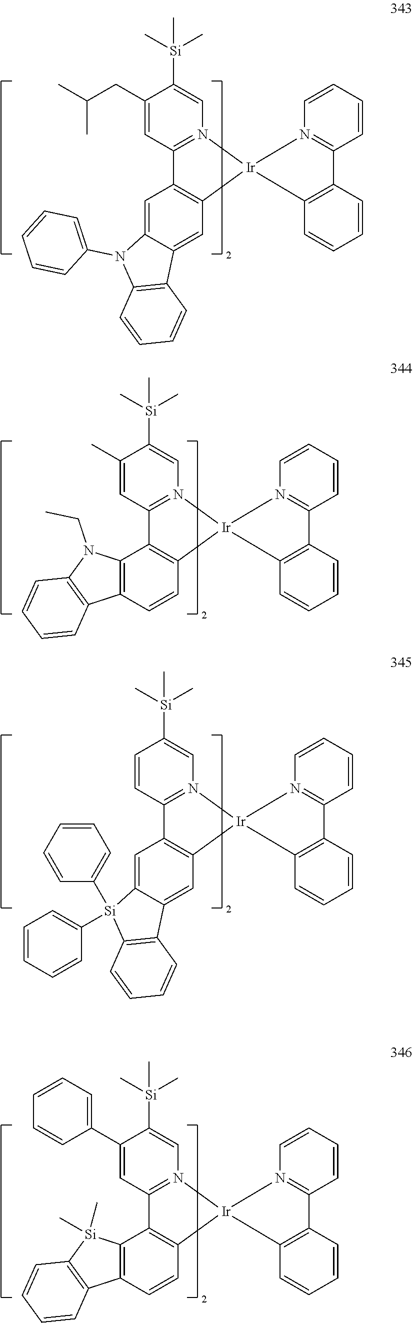

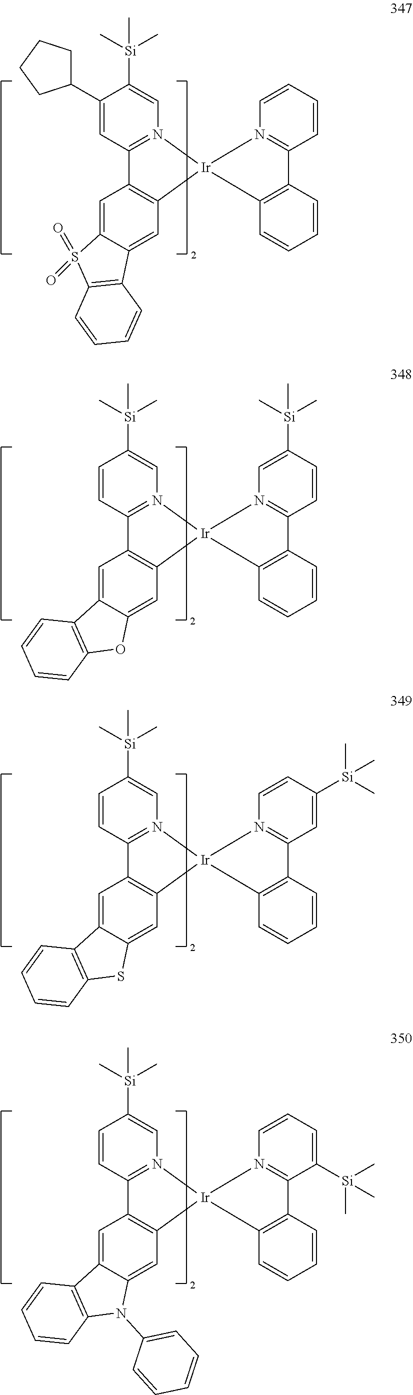

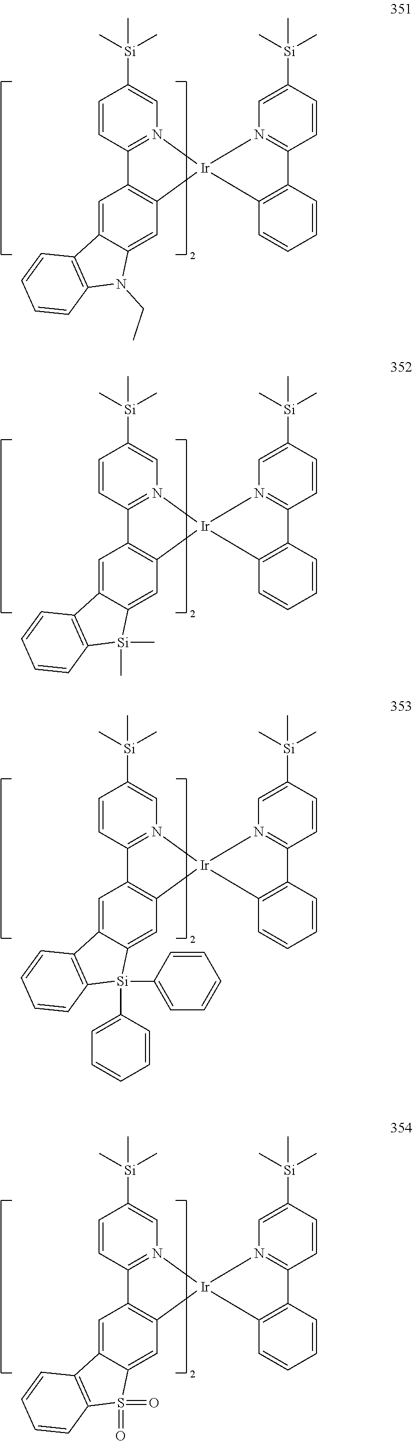

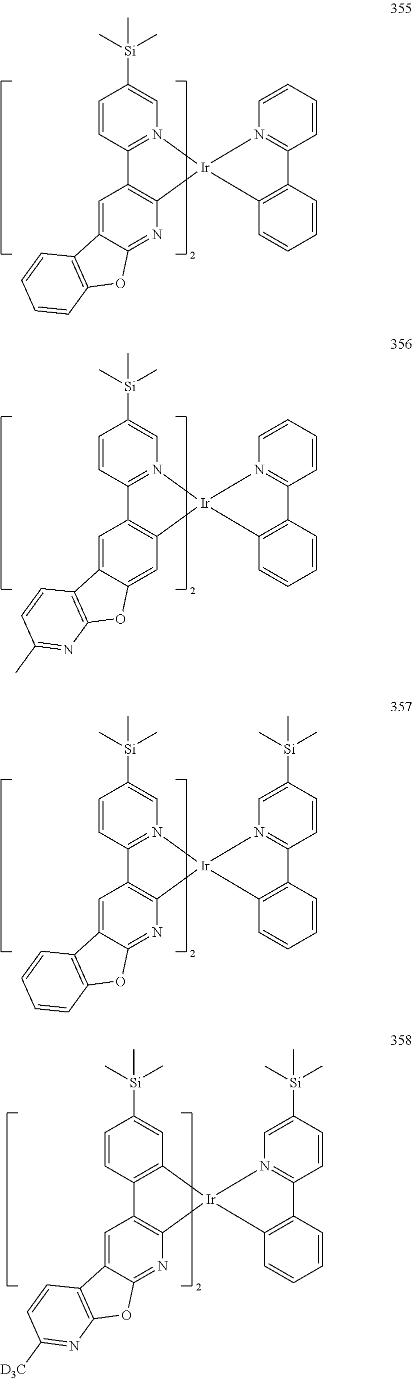

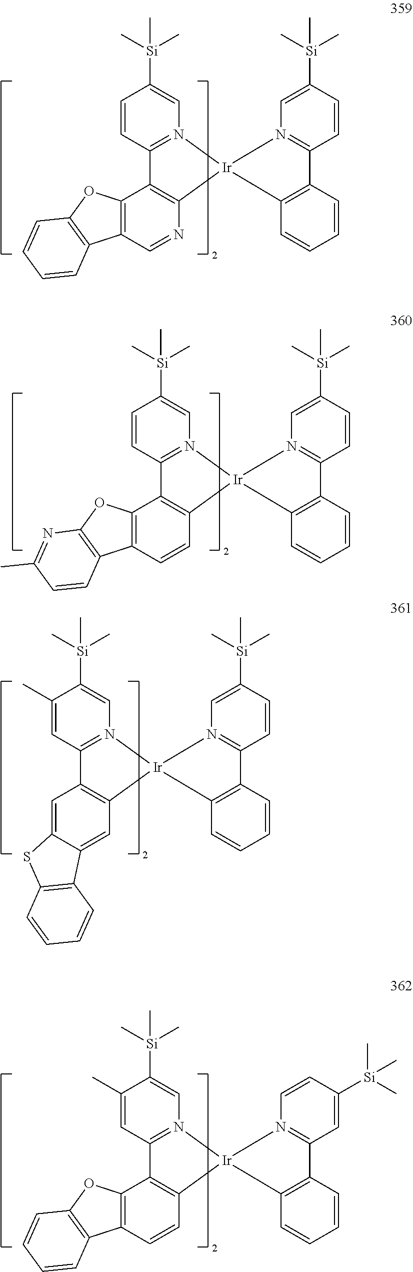

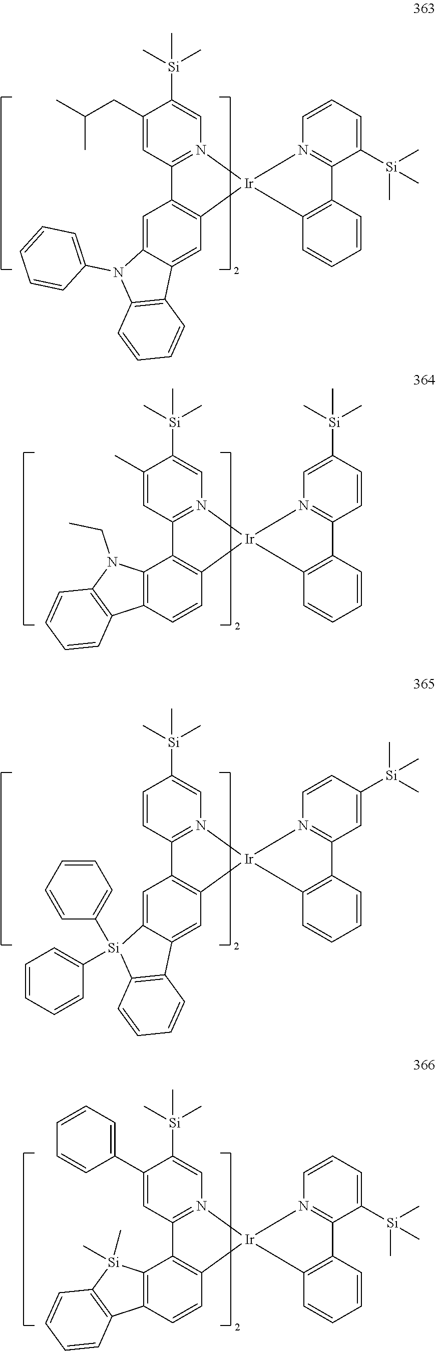

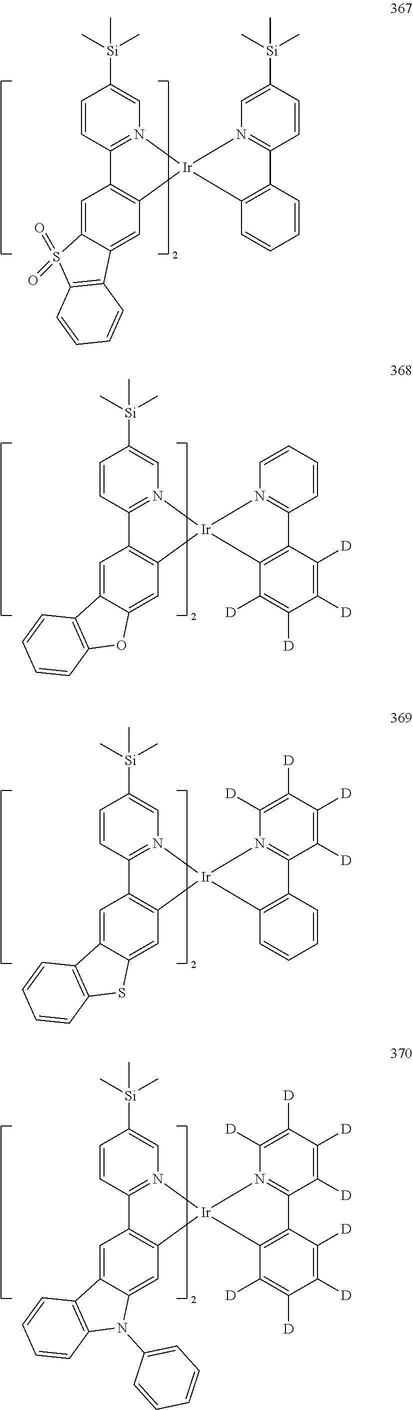

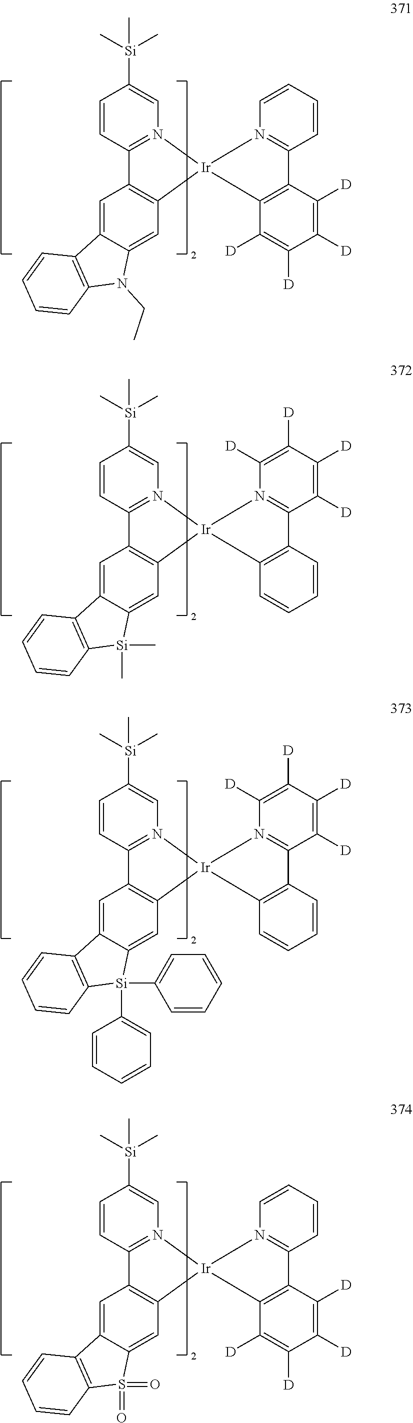

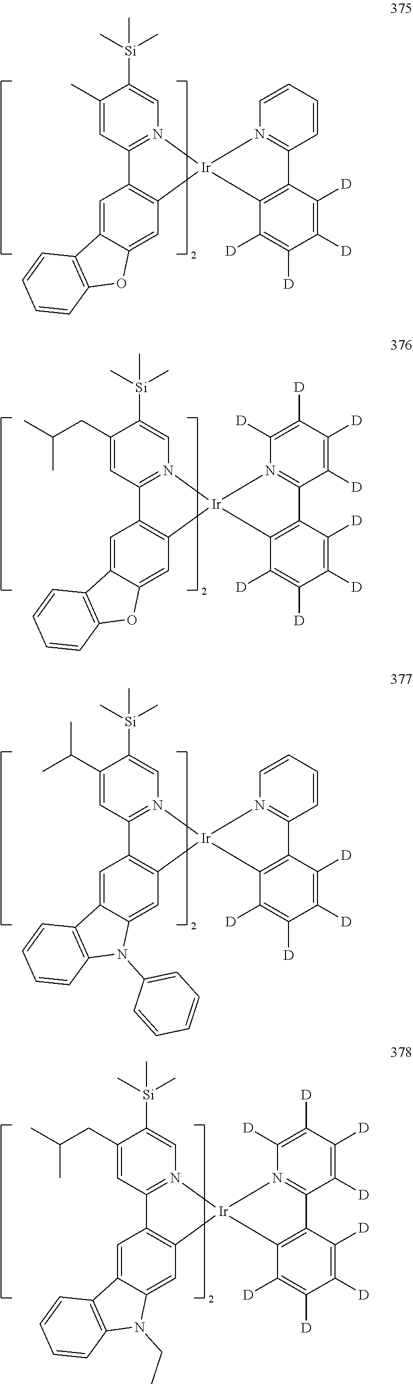

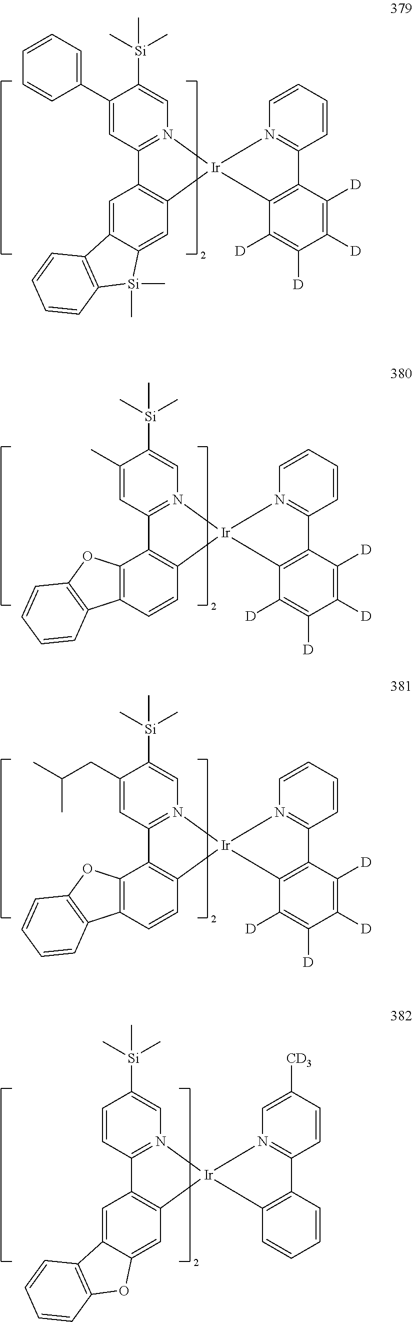

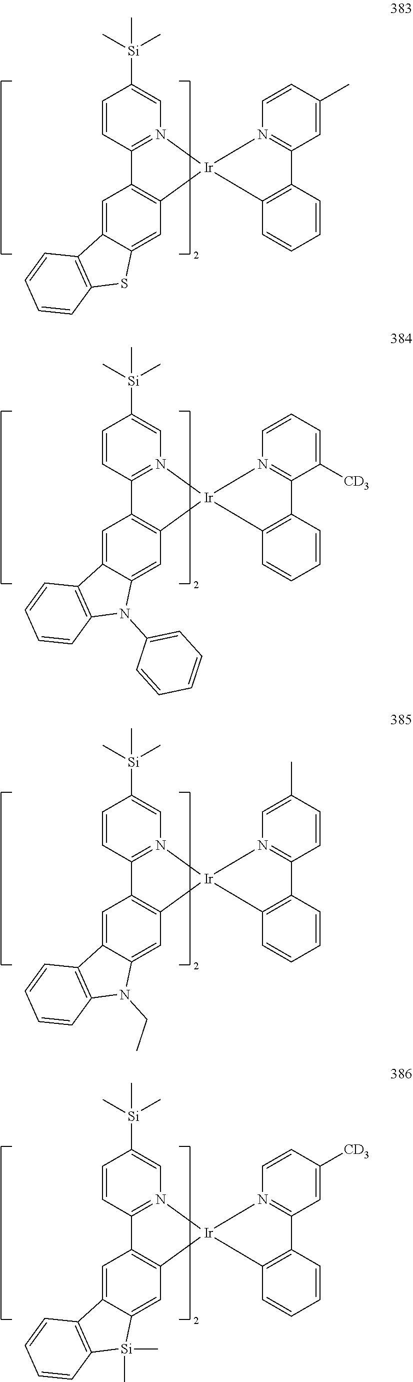

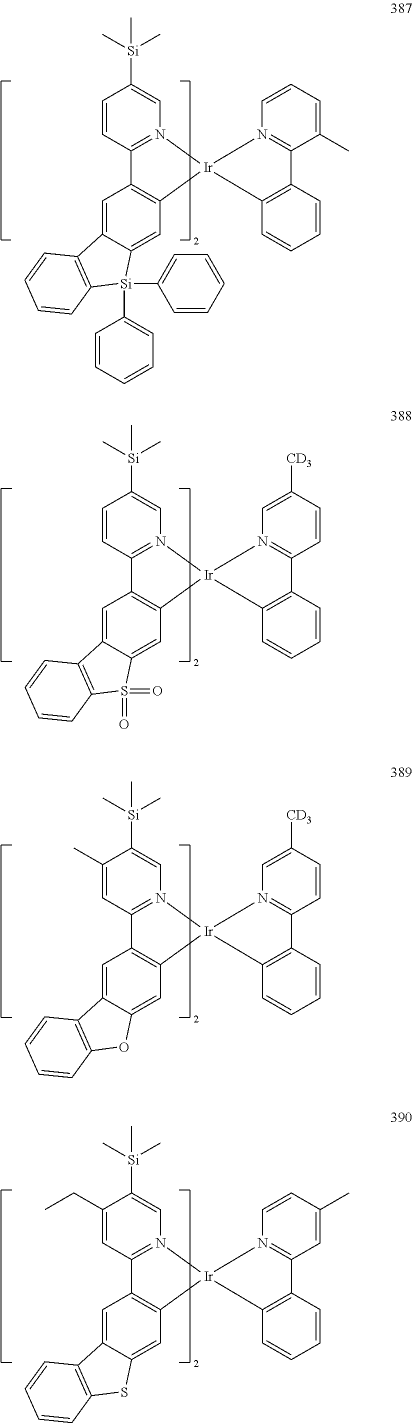

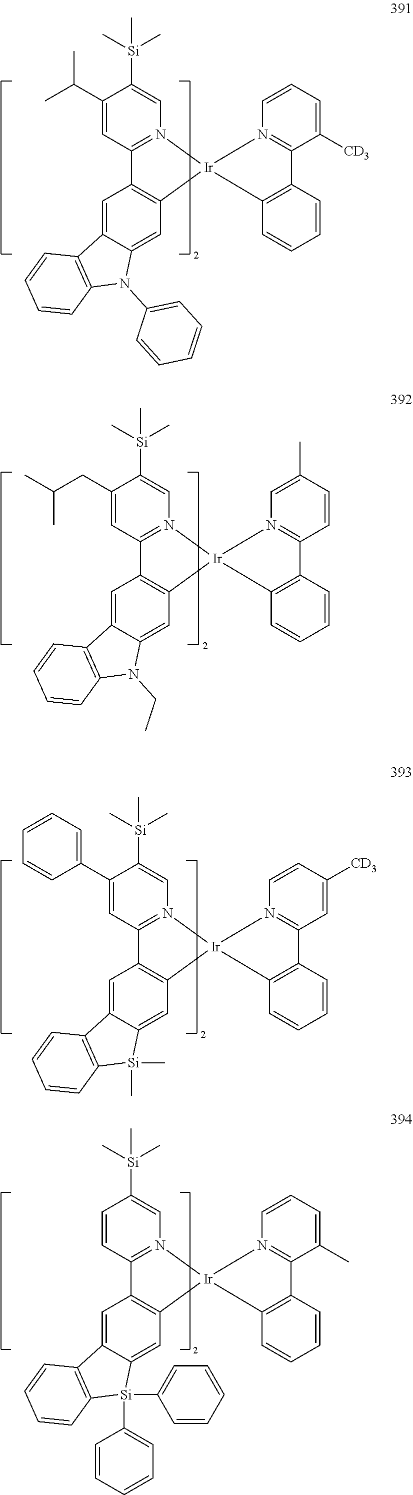

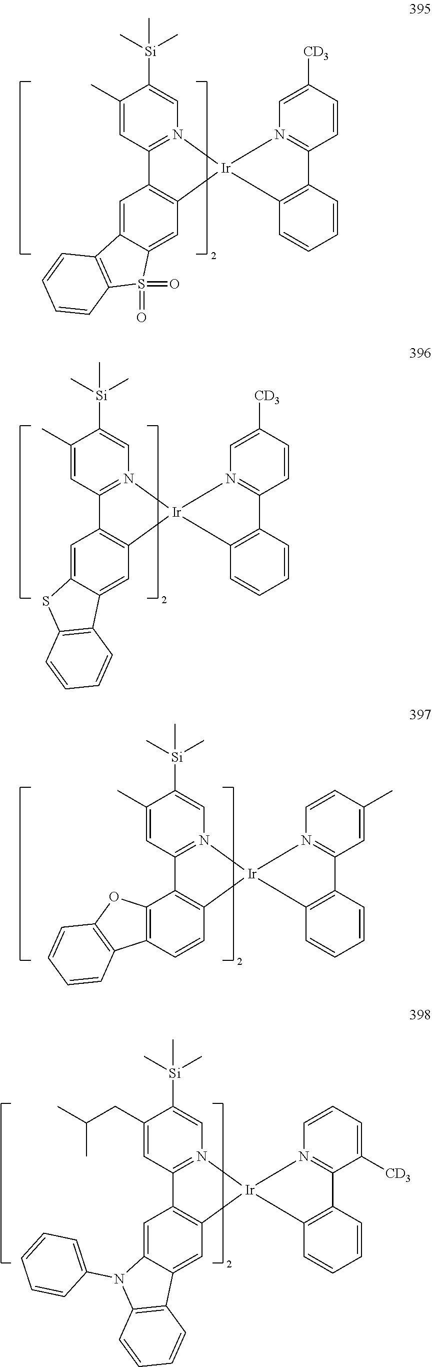

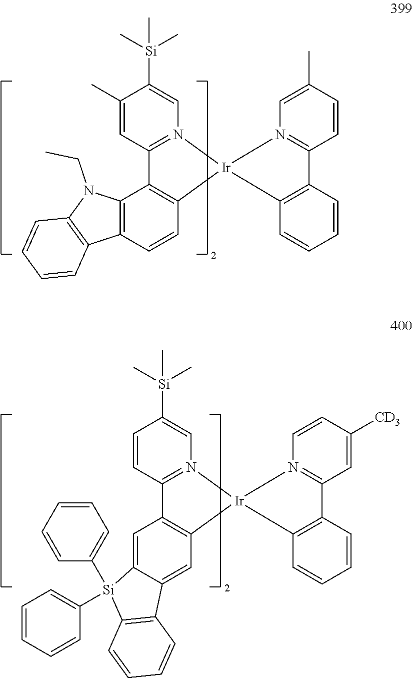

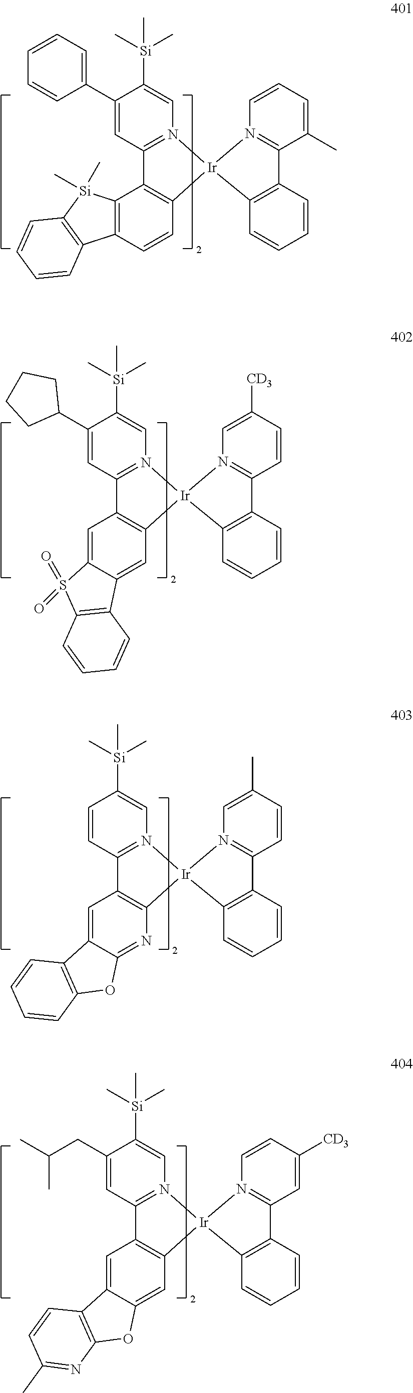

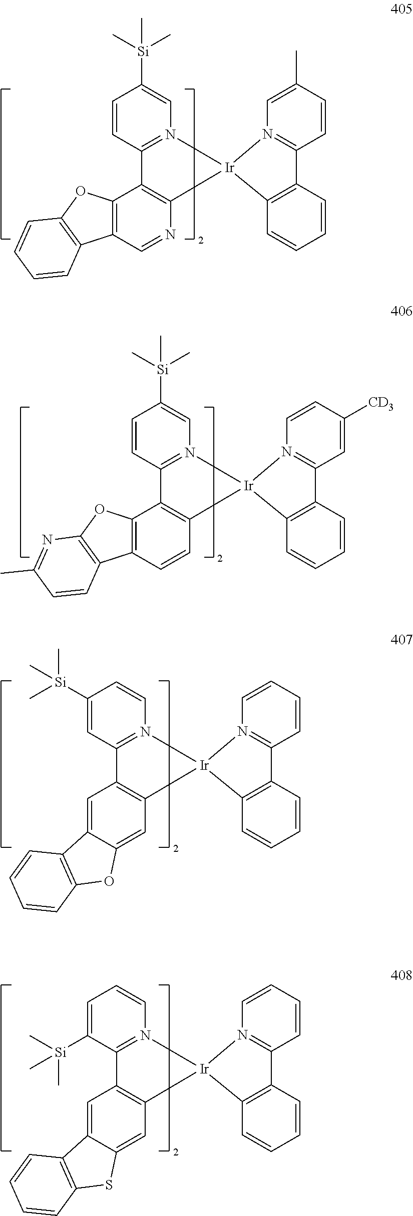

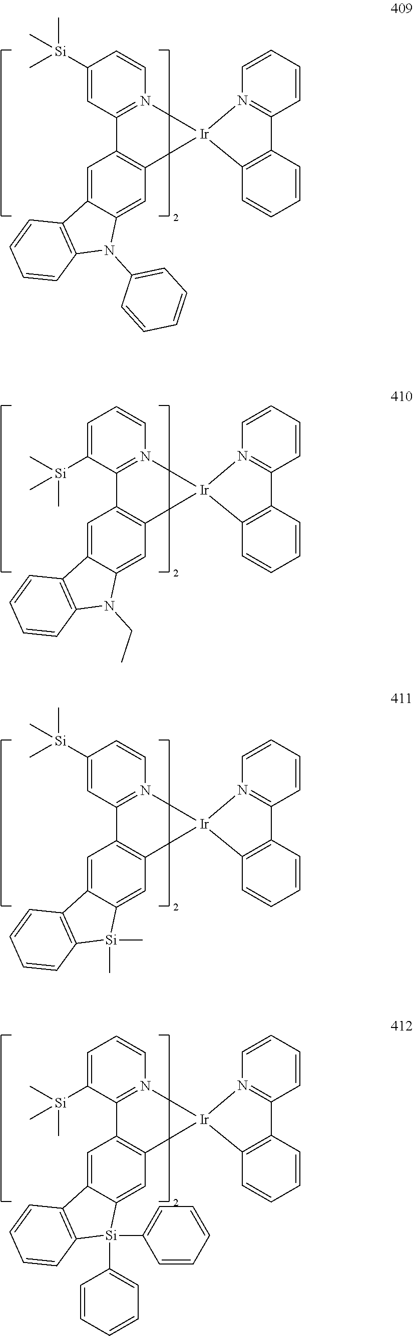

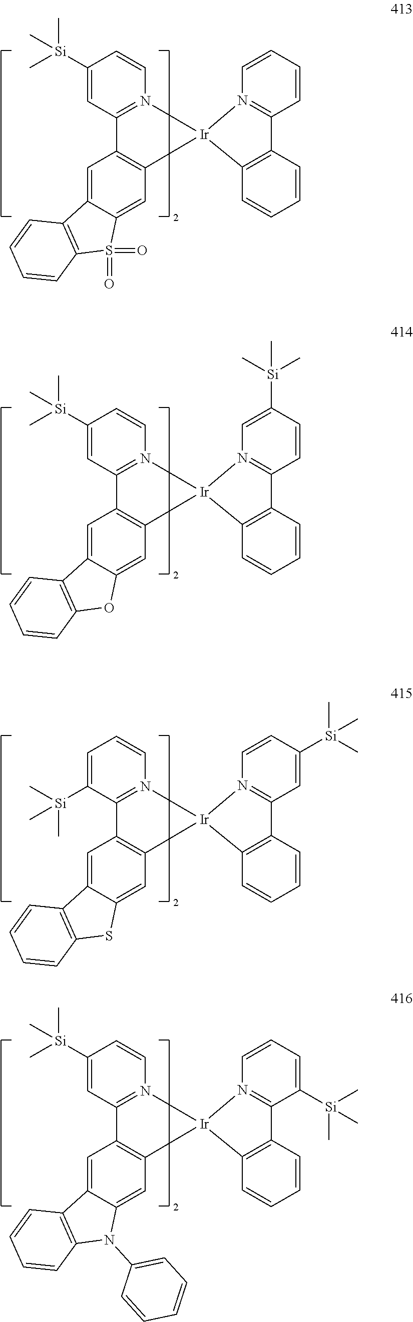

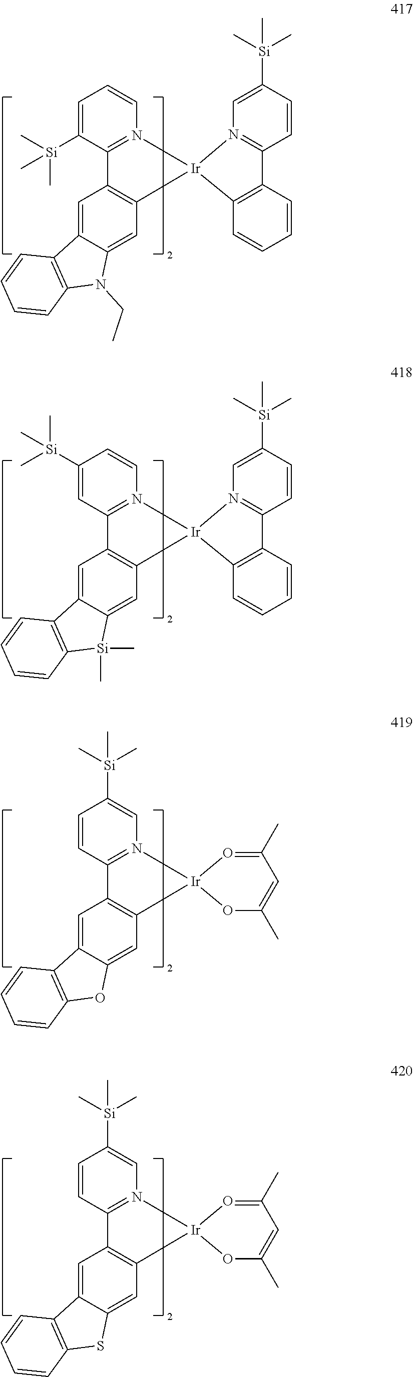

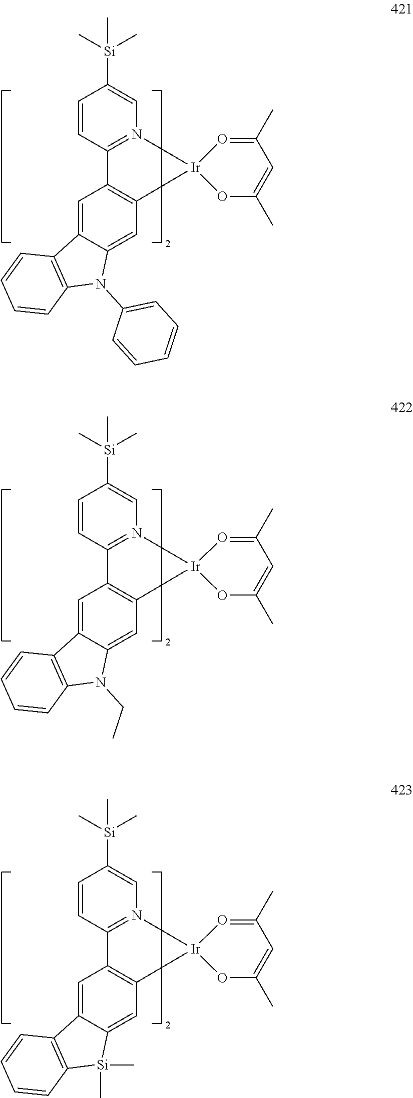

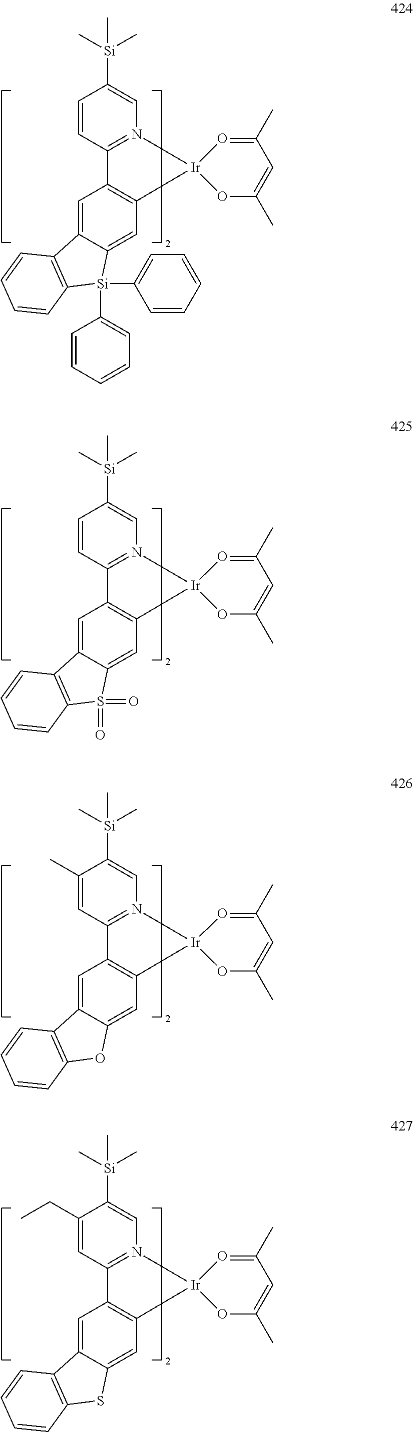

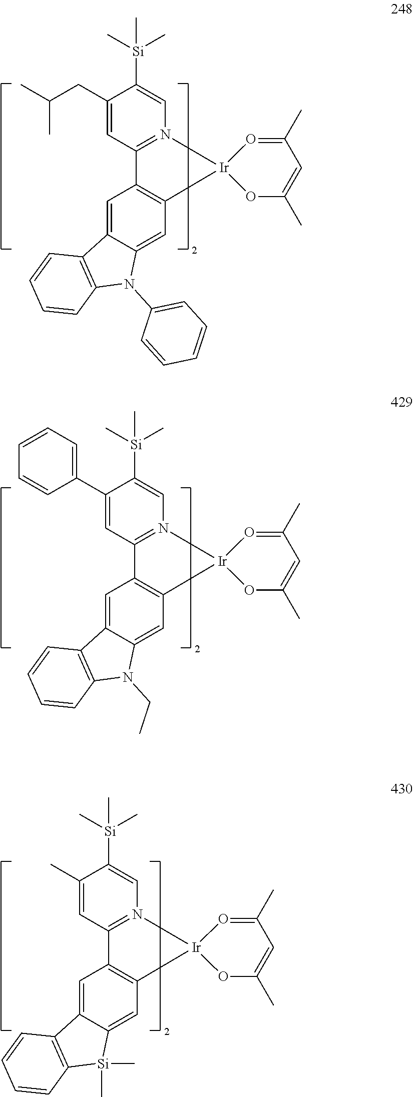

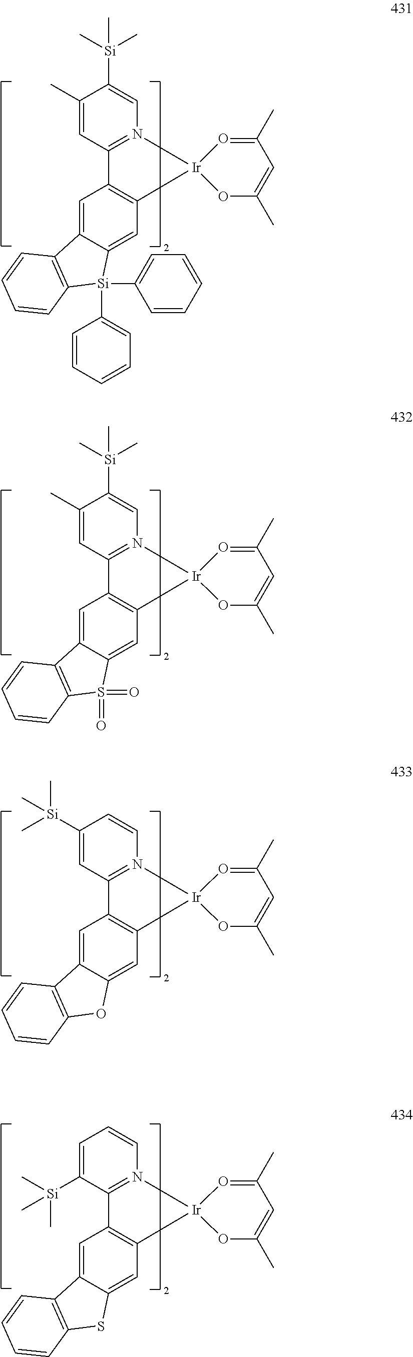

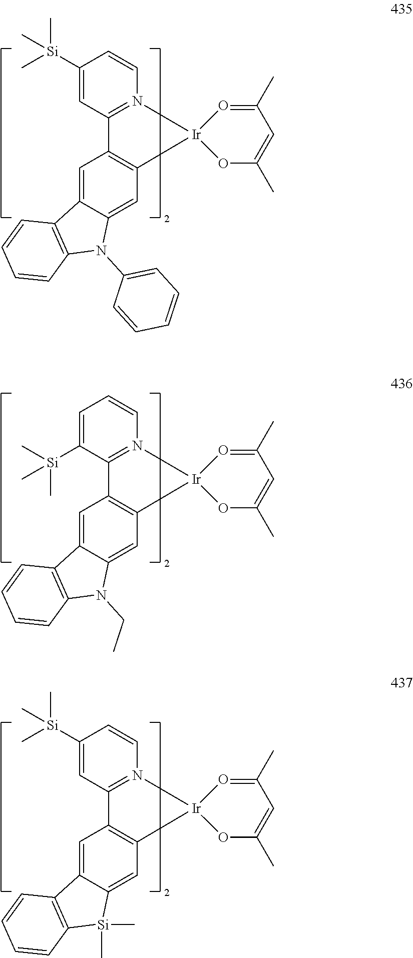

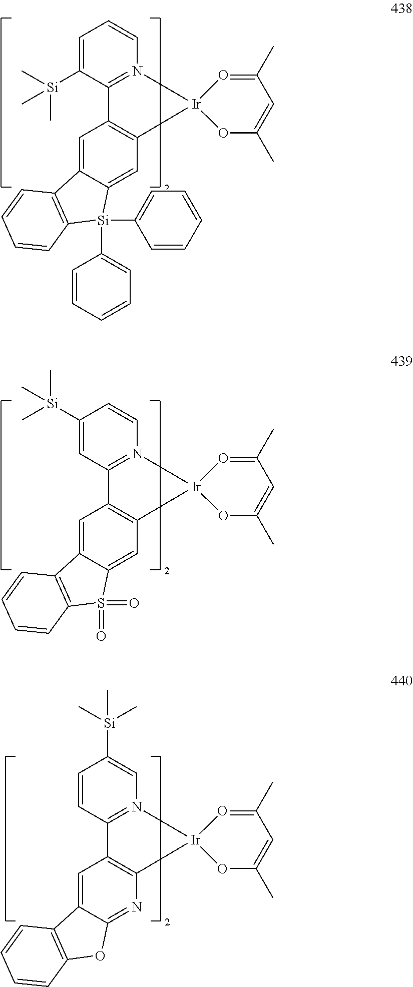

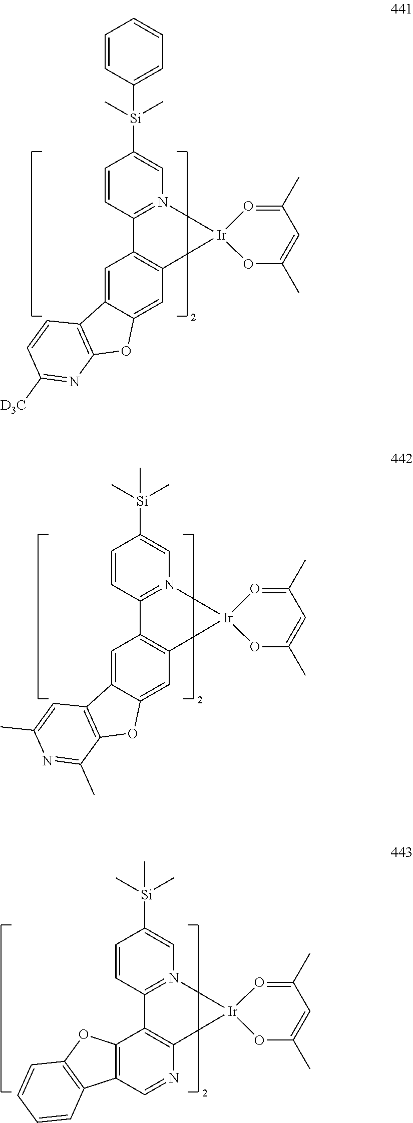

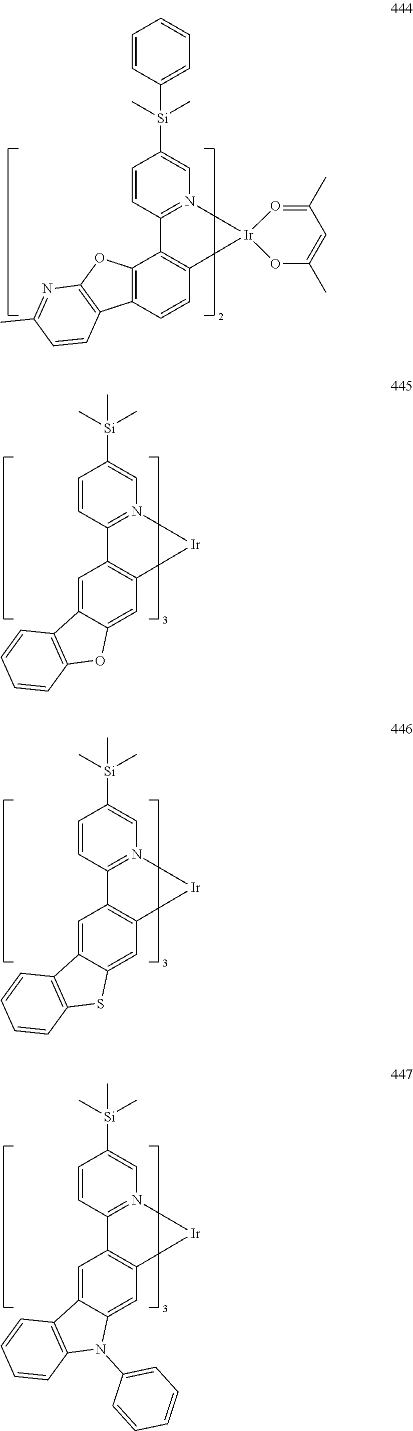

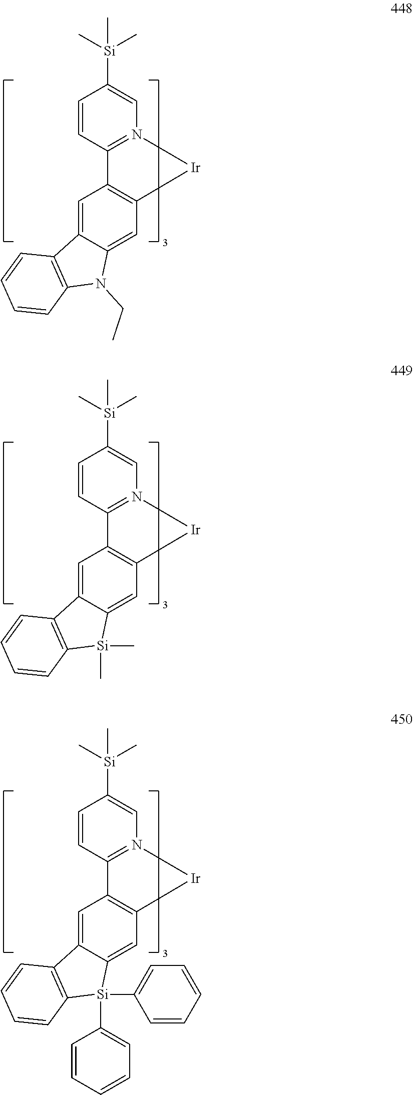

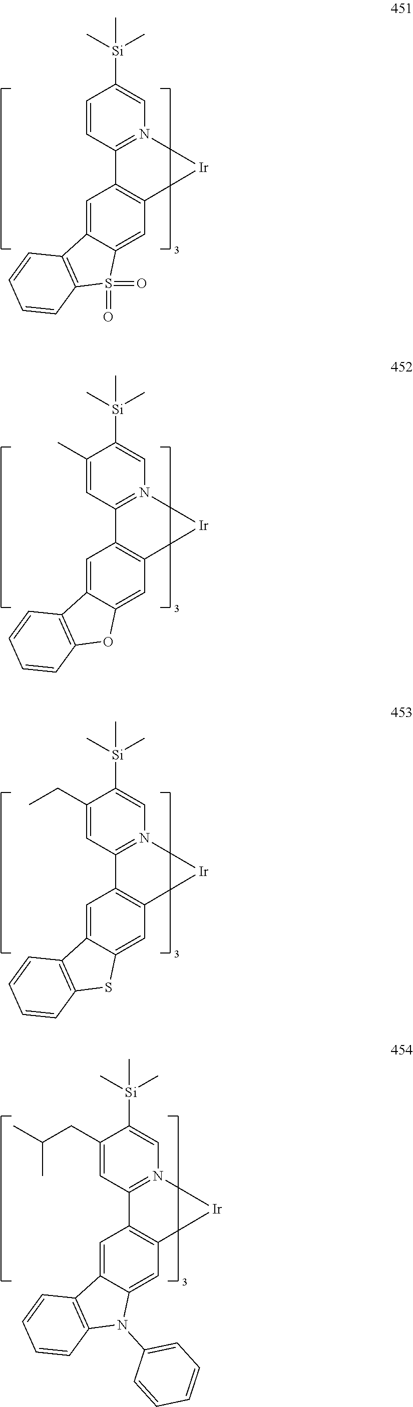

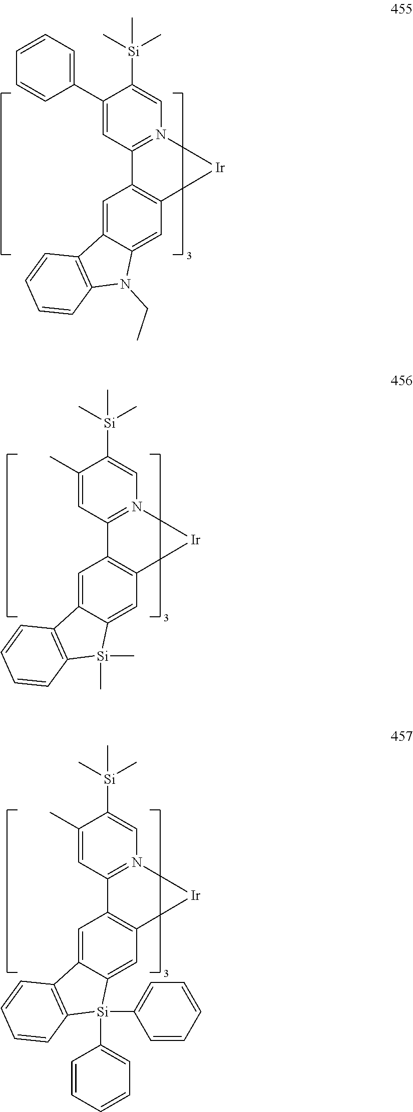

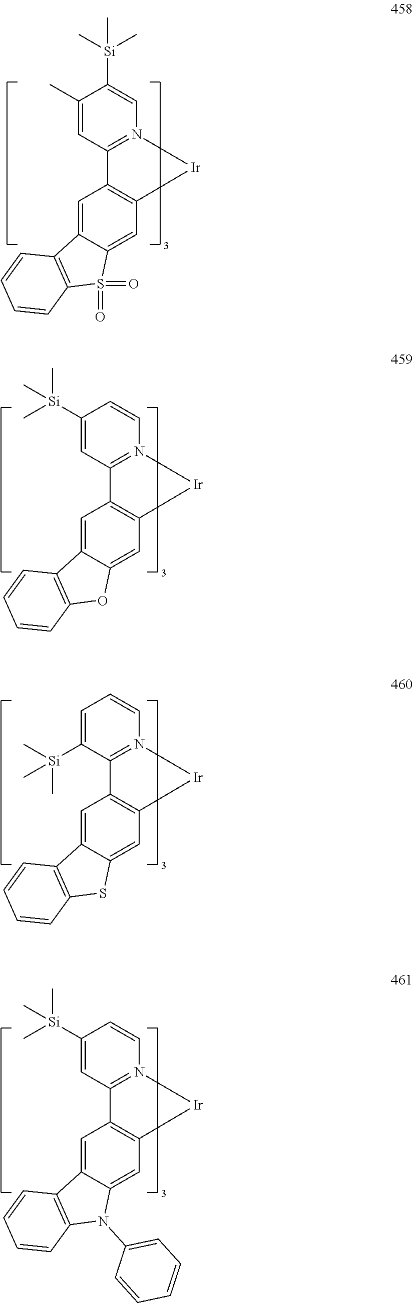

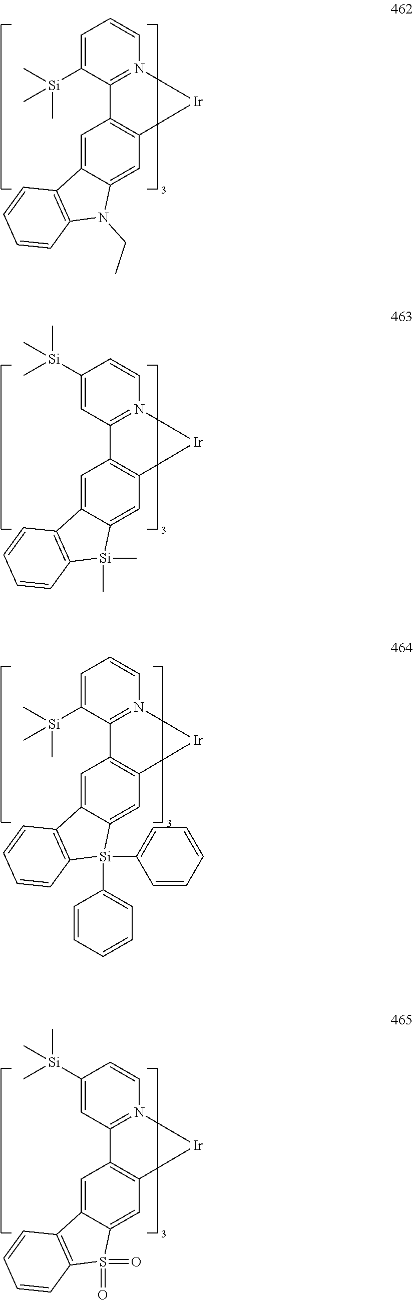

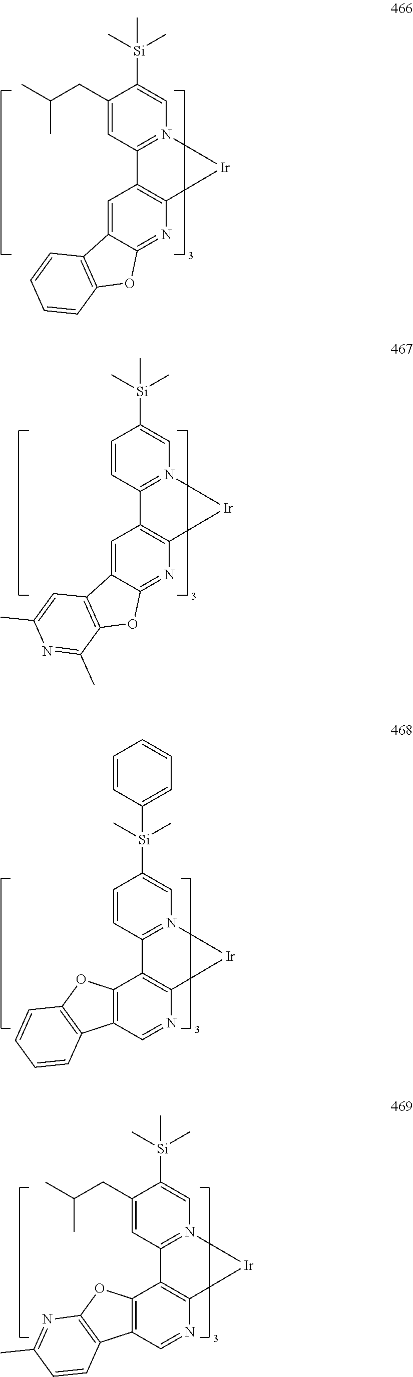

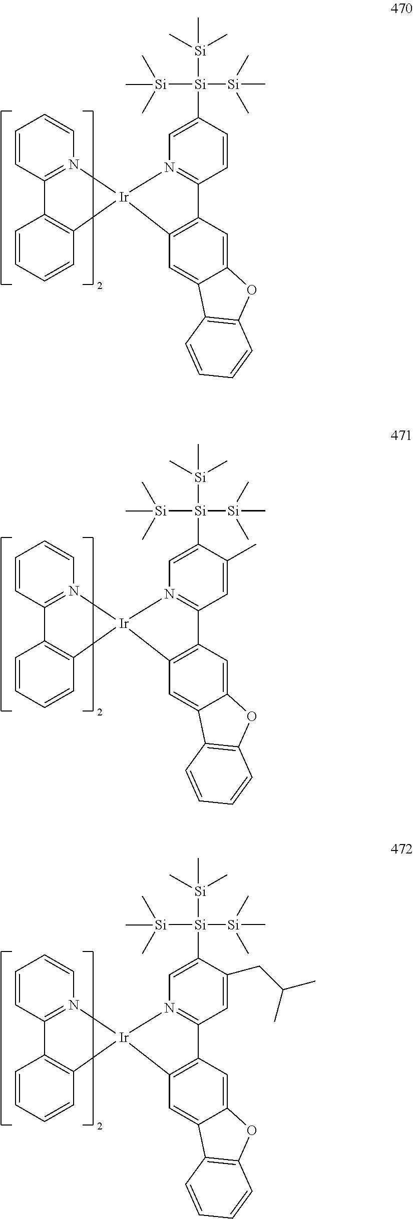

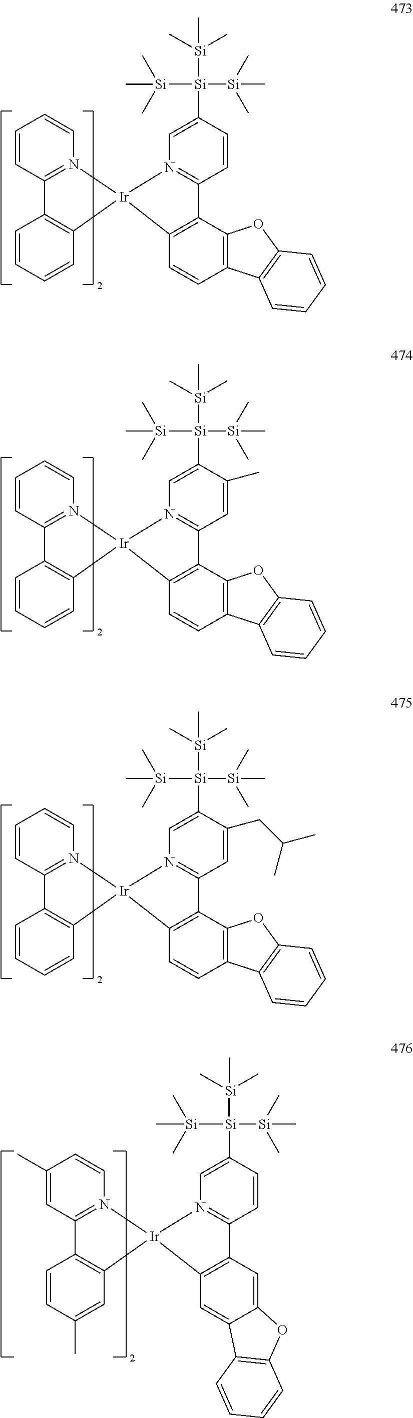

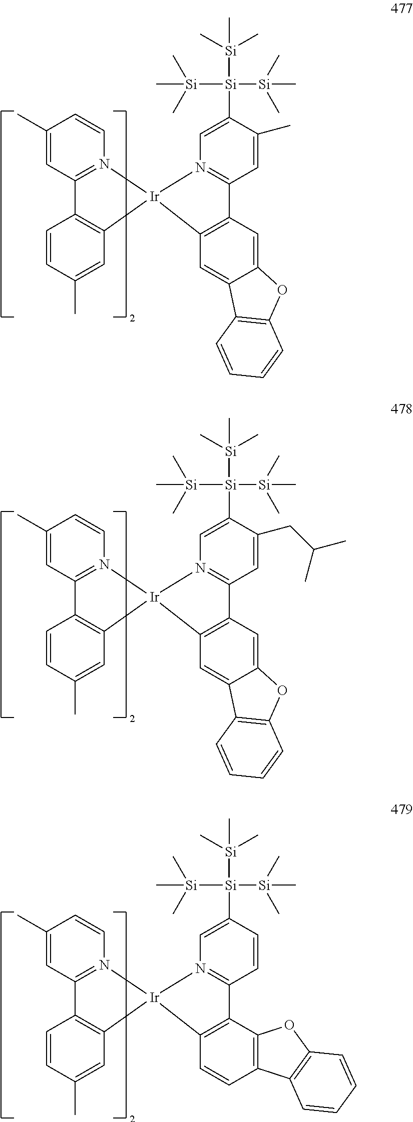

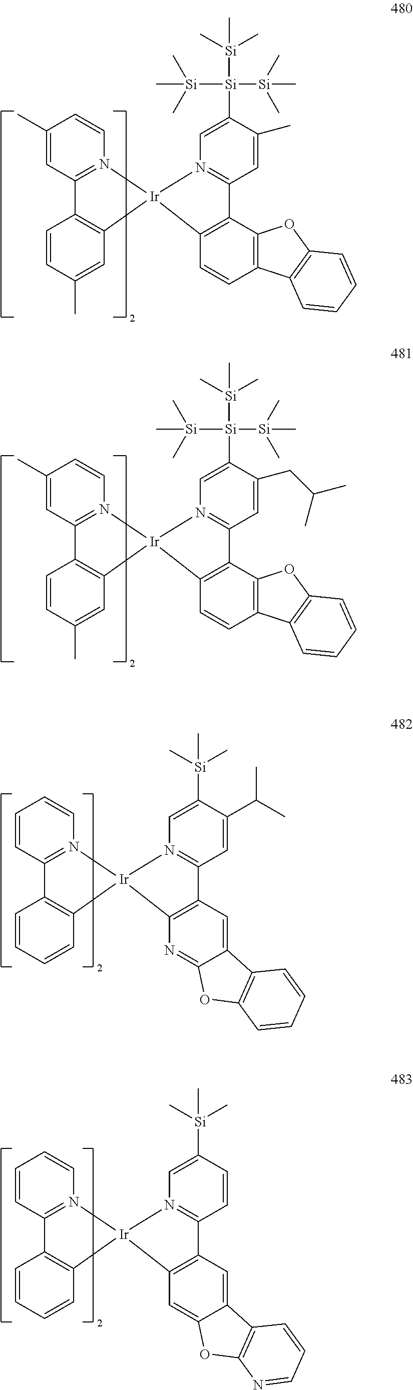

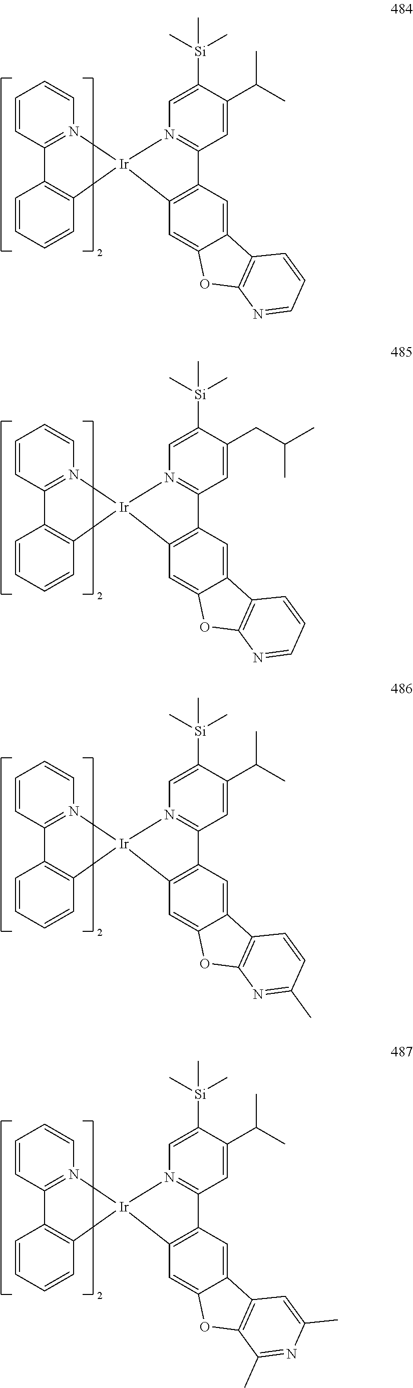

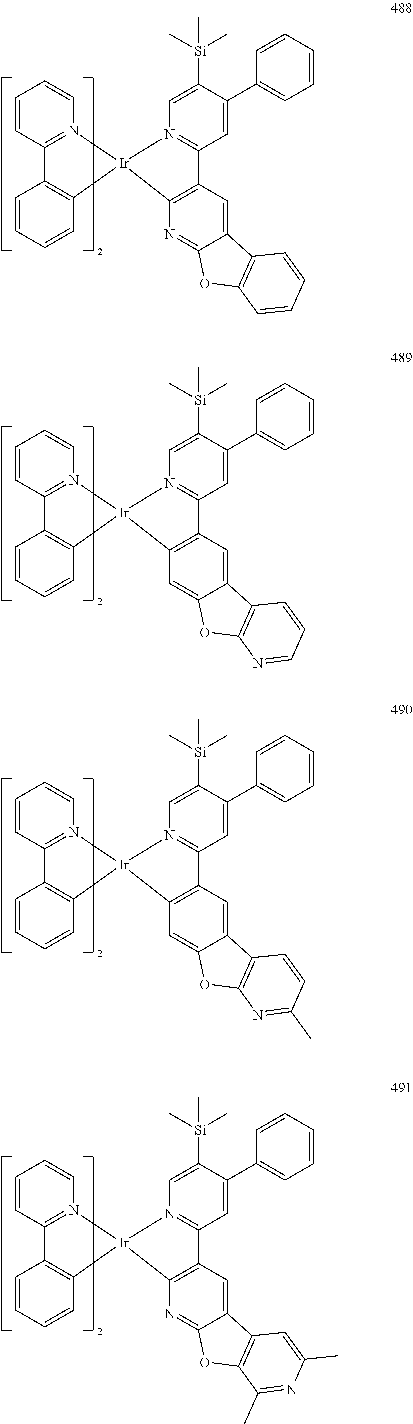

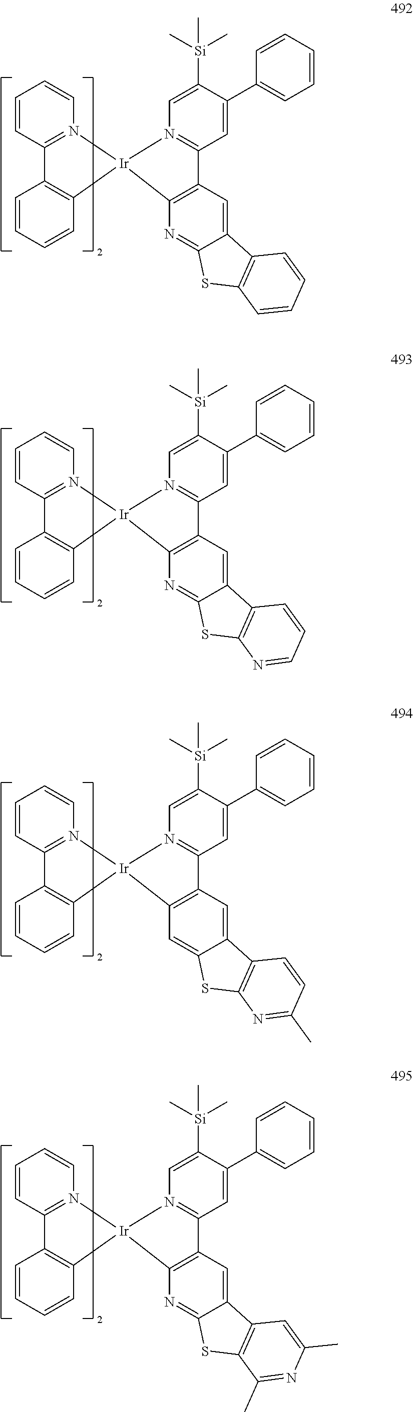

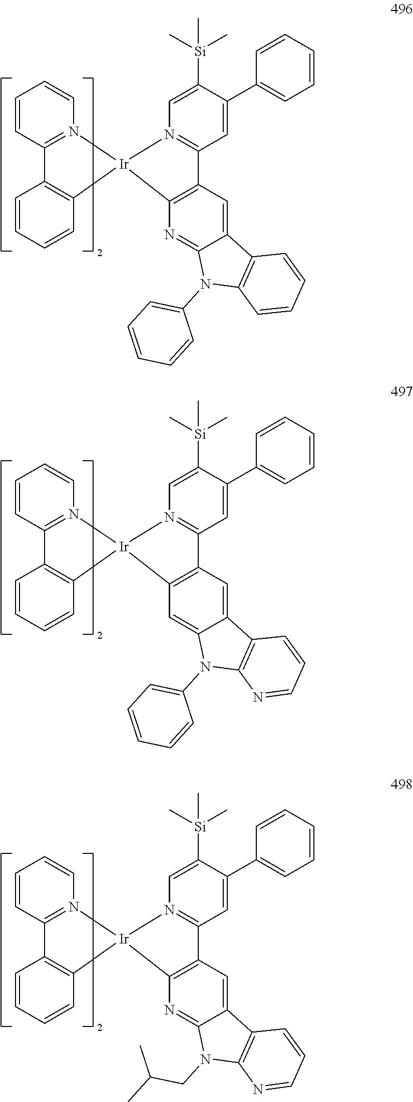

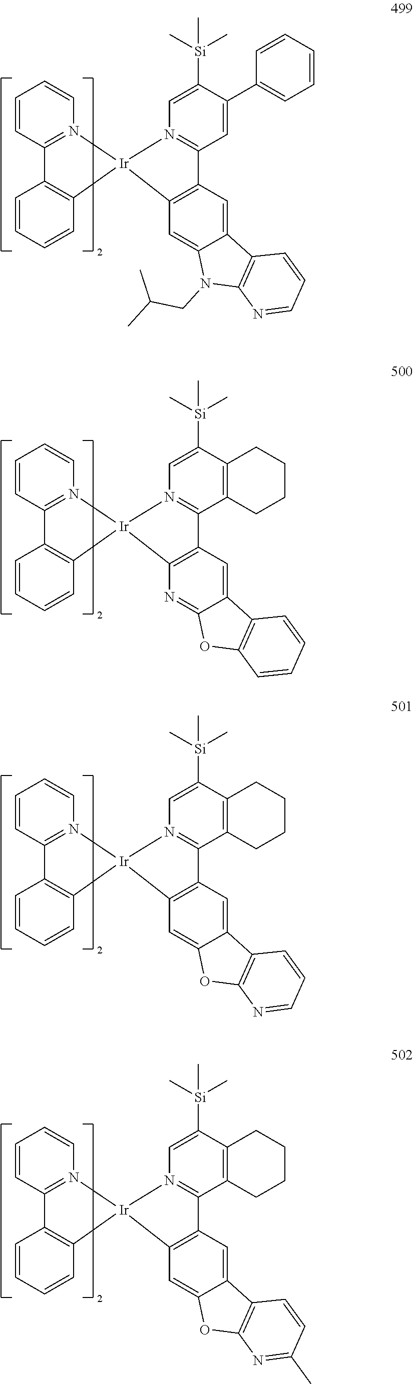

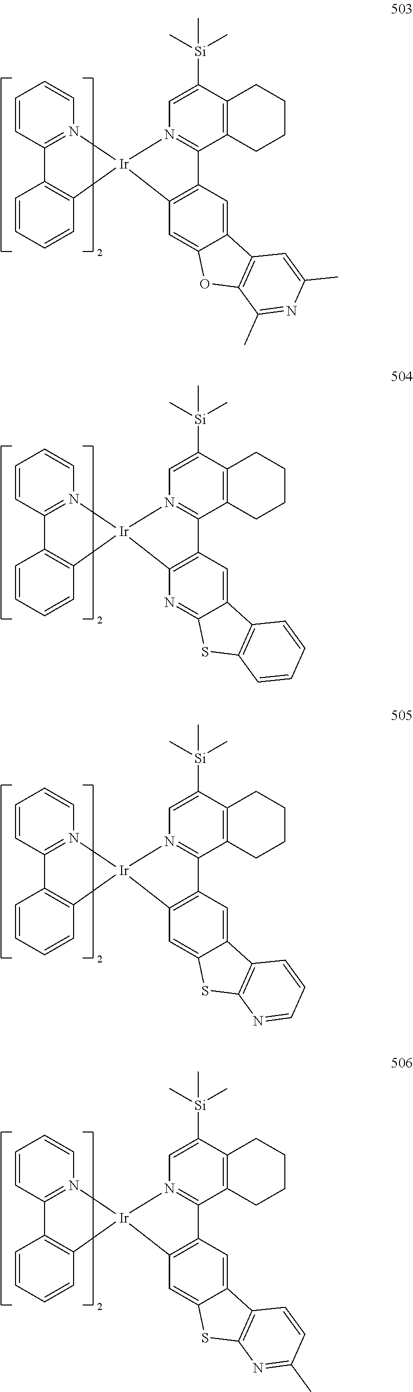

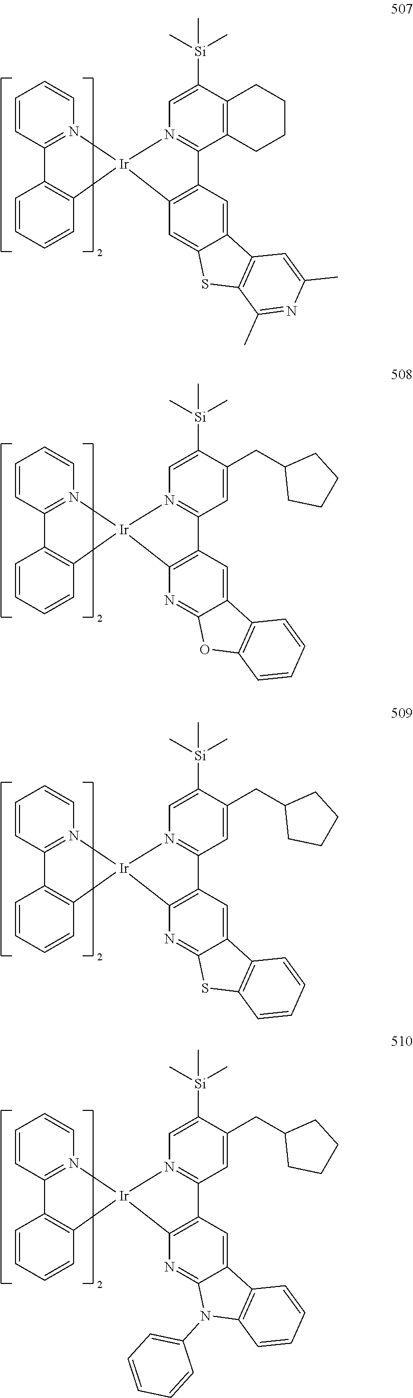

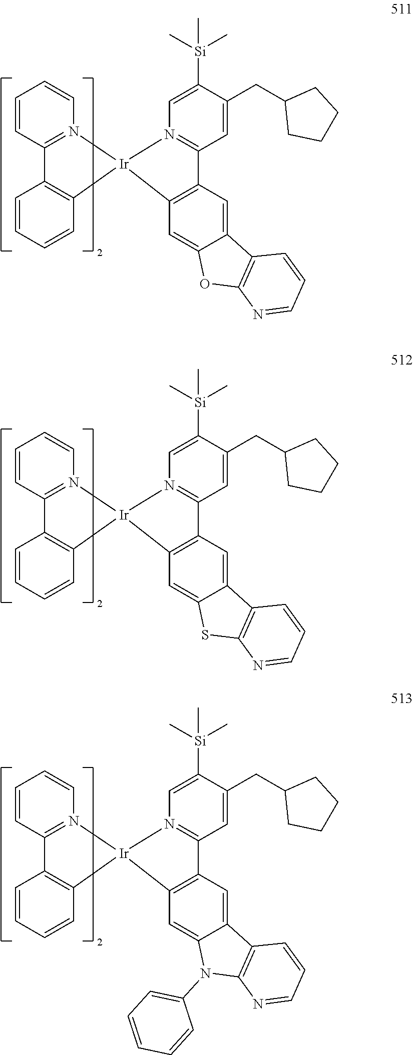

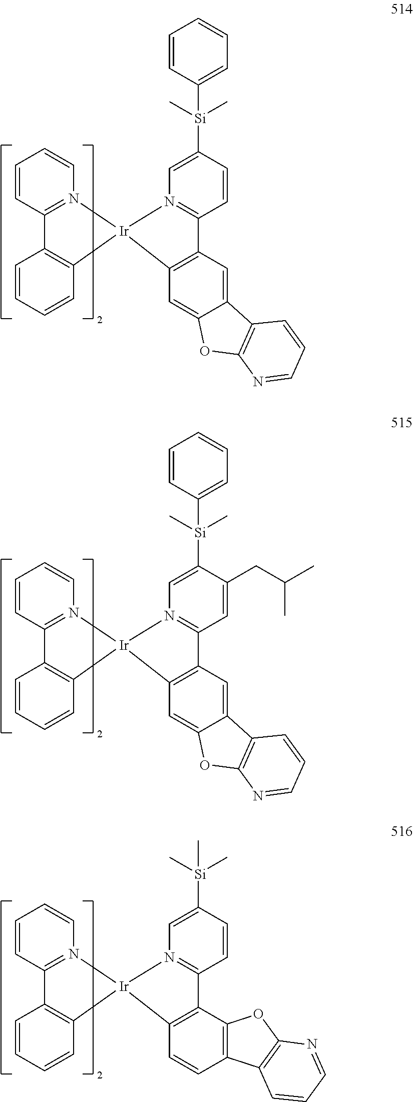

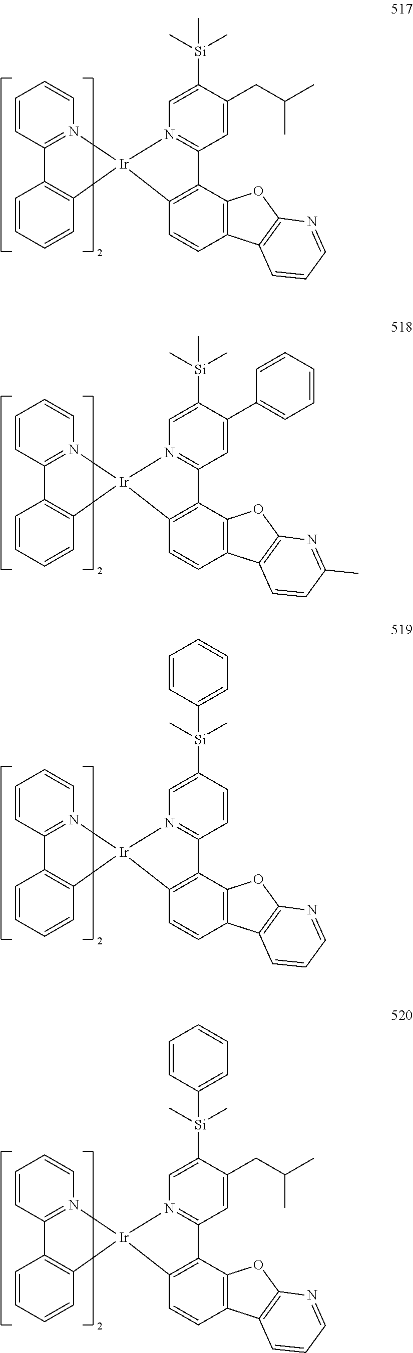

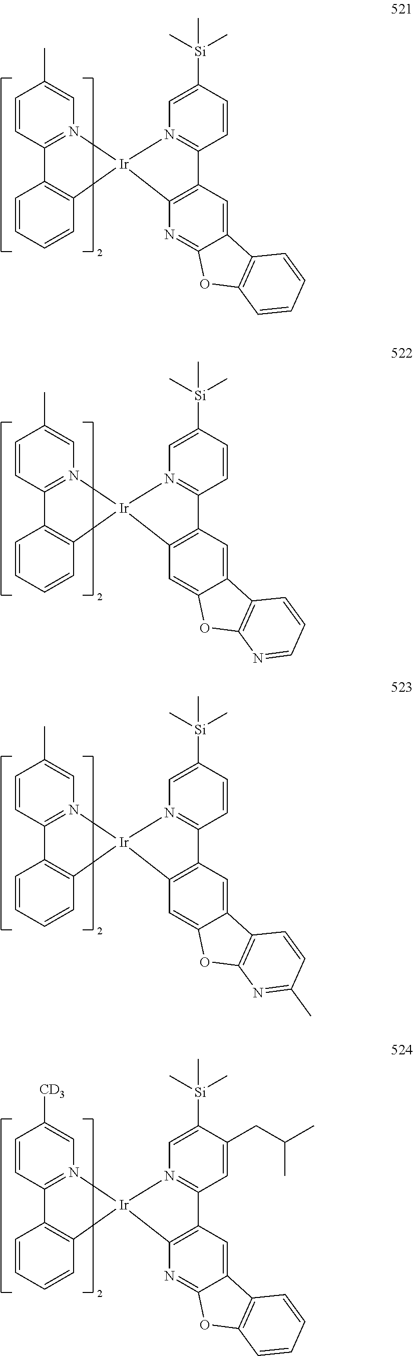

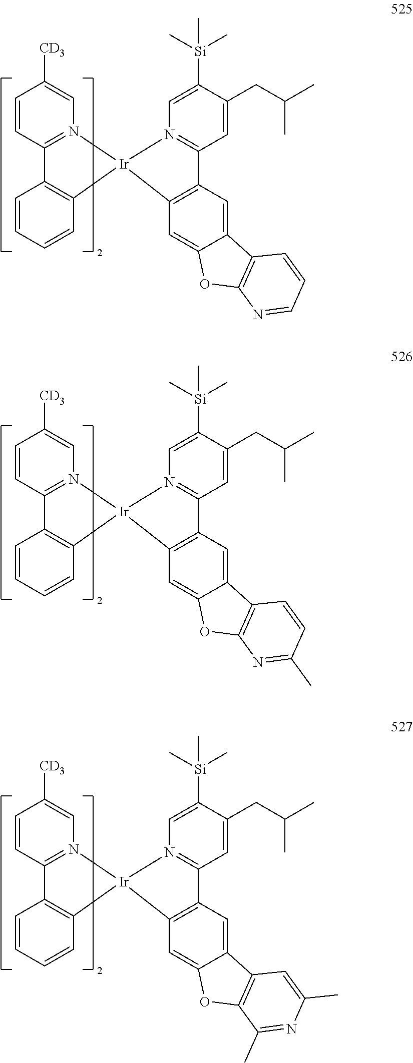

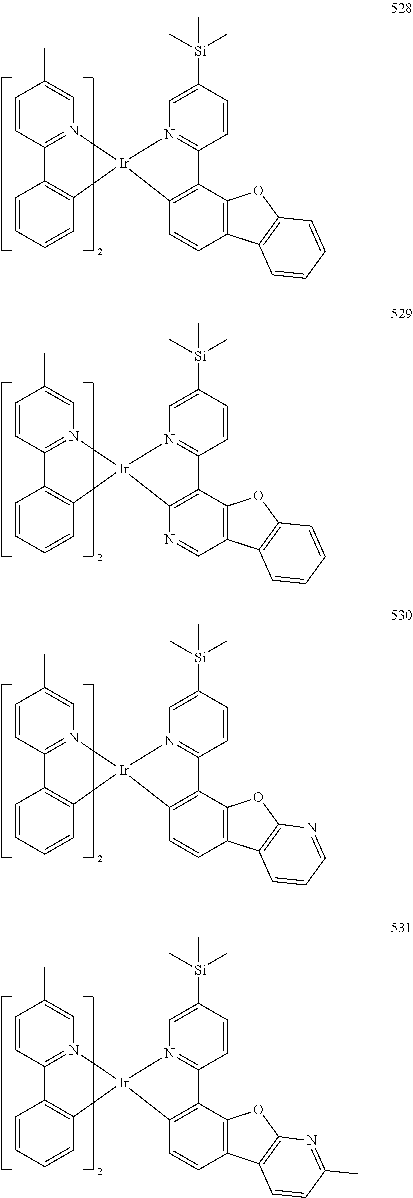

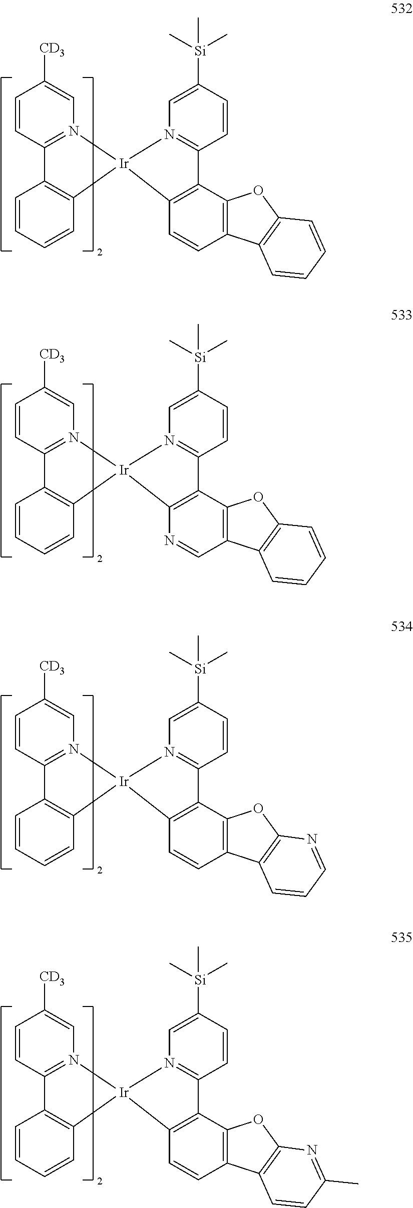

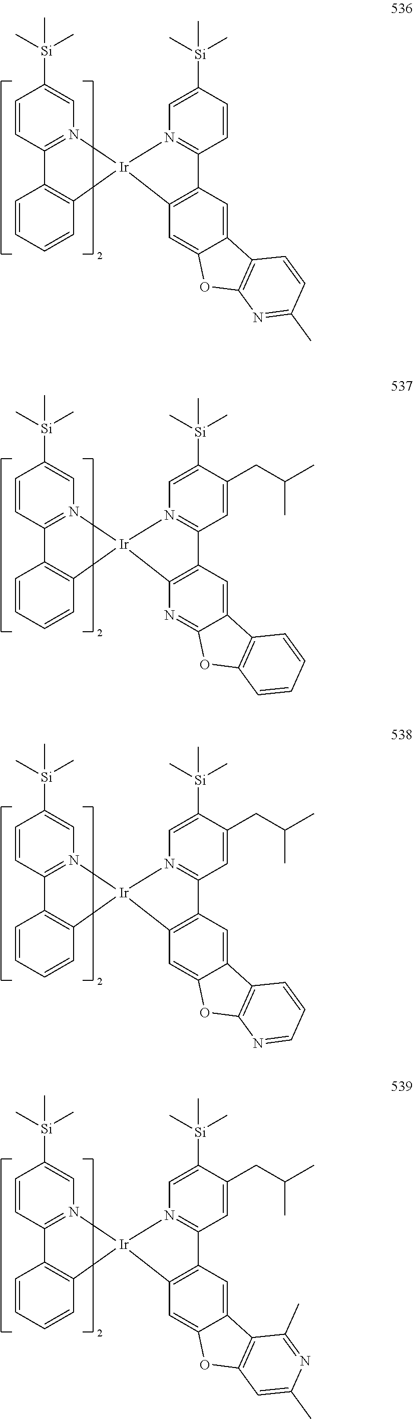

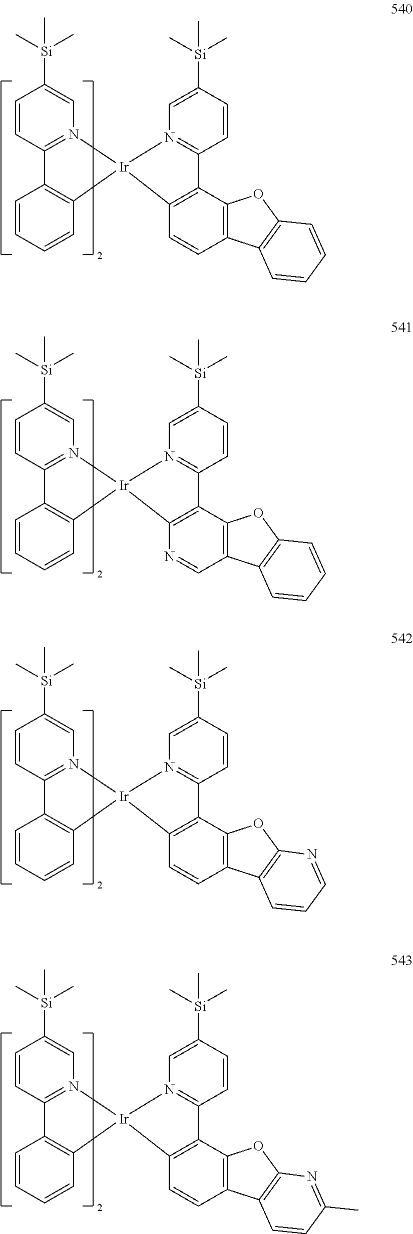

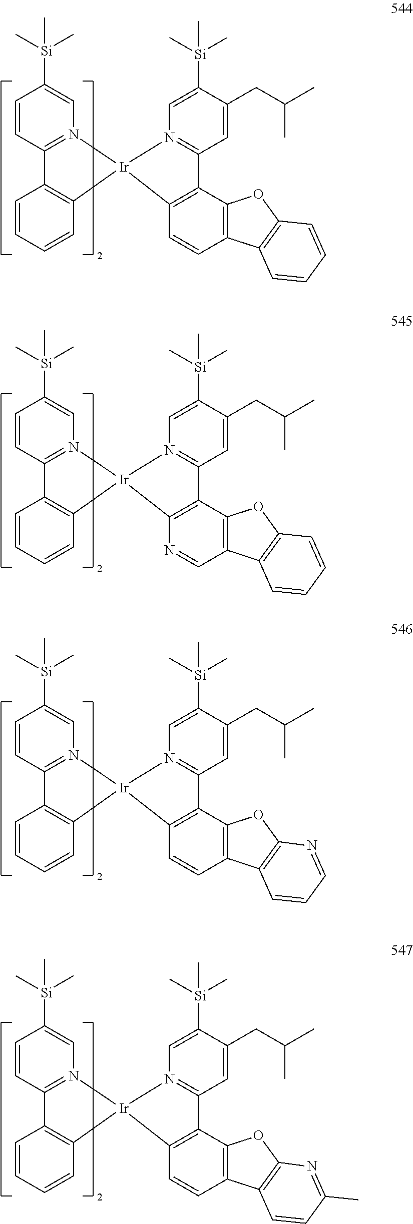

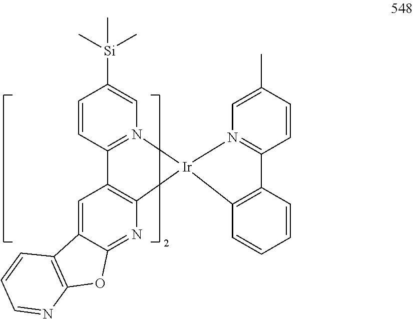

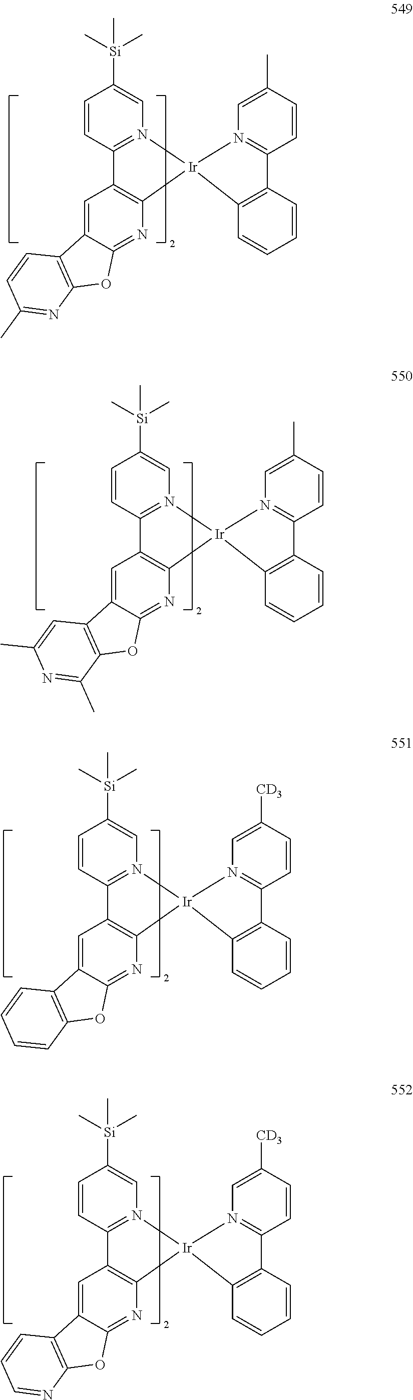

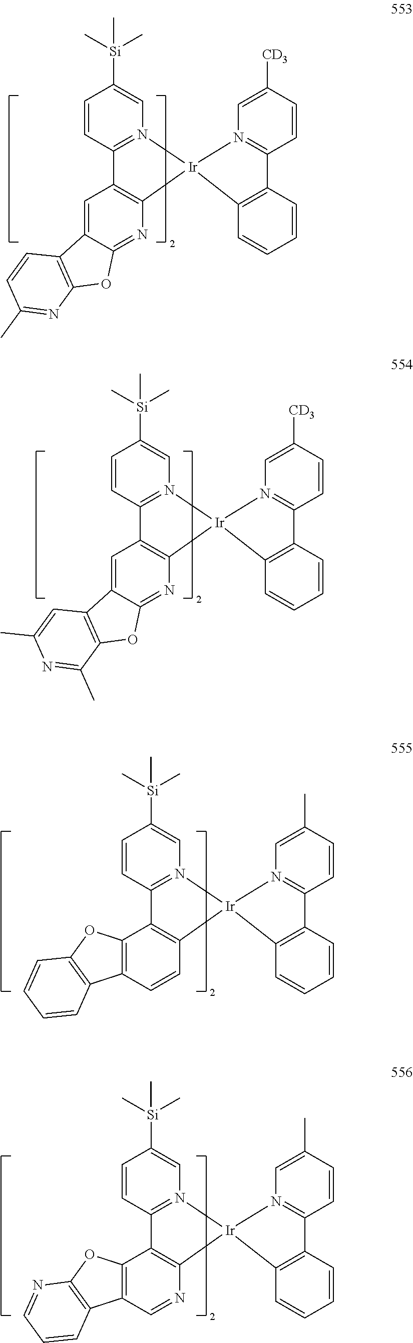

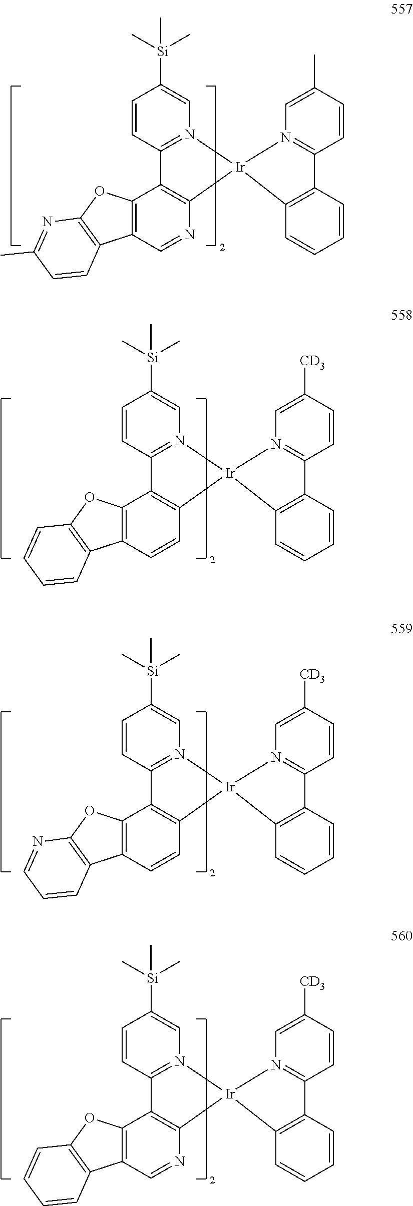

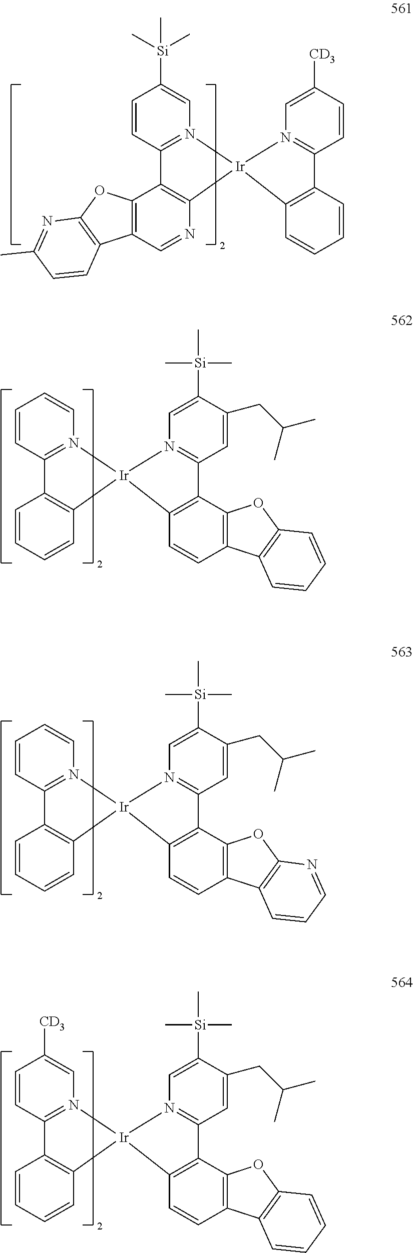

L₁ in the organometallic compound represented by Formula 1 may be selected from ligands represented by Formula 2A, and b4 in Formula 2A may be one or more. That is, a substituent of a pyridine-based ring in Formula 2A may include at least one —Si(R₁)(R₂)(R₃). Since —Si(R₁)(R₂)(R₃) increases a spin density of a metal M of Formula 1, the organic light-emitting device including the organometallic compound represented by Formula 1, which includes the ligand represented by Formula 2A, may have high efficiency.

X₁ in Formula 2A may be selected from O, S, S(═O)₂, N(R₂₁), and Si(R₂₂)(R₂₃). Thus, charge mobility of the organometallic compound including the ligand represented by Formula 2A is improved and energy level adjustment is facilitated, thereby improved the efficiency and lifespan of the organic light-emitting device including the organometallic compound.

For example, the highest occupied molecular orbital (HOMO) energy level, lowest unoccupied molecular orbital (LUMO) energy level, singlet (S₁) energy level, and triplet (T₁) energy level of Compounds 1, 2, 8, 22, 139, 146, 304, 305, 321, 323, 327, and 419 were evaluated by a density functional theory (DFT) method of a Gaussian program (the structure was optimized at B3LYP, 6-31G(d,p) level). Results thereof are shown in Table 1.

TABLE 1

| | | | S₁ energy | T₁ energy |
|---|---|---|---|---|
| Compound No. | HOMO (eV) | LUMO (eV) | level (eV) | level (eV) |
| Compound 1 | −4.842 | −1.262 | 2.887 | 2.616 |
| Compound 2 | −4.874 | −1.309 | 2.869 | 2.591 |
| Compound 8 | −4.812 | −1.249 | 2.875 | 2.608 |
| Compound 22 | −4.812 | −1.250 | 2.889 | 2.633 |
| Compound 139 | −4.827 | −1.283 | 2.867 | 2.589 |
| Compound 146 | −4.829 | −1.261 | 2.872 | 2.510 |
| Compound 304 | −4.781 | −1.267 | 2.837 | 2.586 |
| Compound 305 | −4.773 | −1.244 | 2.852 | 2.600 |
| Compound 321 | −4.856 | −1.369 | 2.803 | 2.555 |
| Compound 323 | −4.820 | −1.281 | 2.849 | 2.591 |
| Compound 327 | −4.863 | −1.304 | 2.887 | 2.614 |
| Compound 419 | −4.756 | −1.331 | 2.753 | 2.525 |

Compound 1 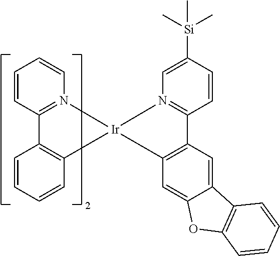
Compound 2 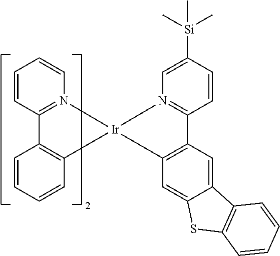
Compound 8 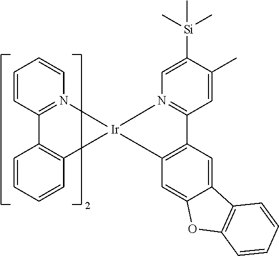
Compound 22 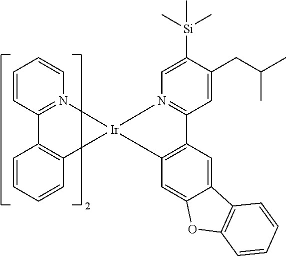
Compound 139 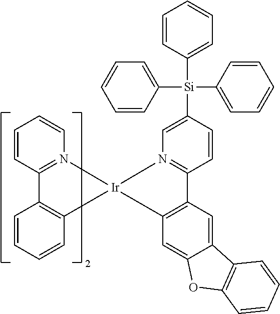
Compound 146 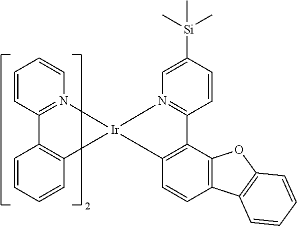
Compound 304 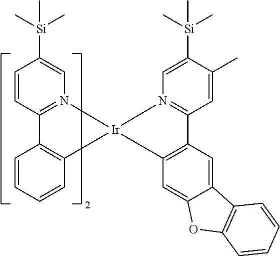
Compound 305 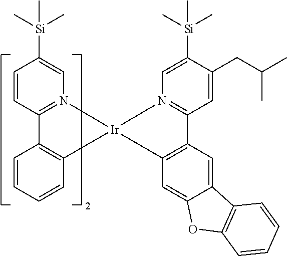
Compound 321 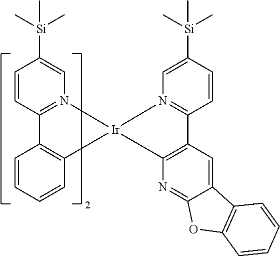
Compound 323 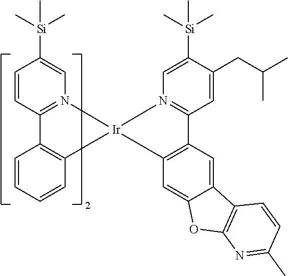
Compound 327 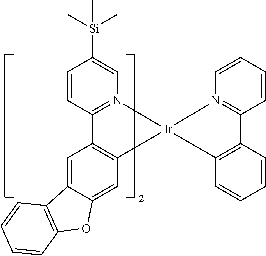
Compound 419

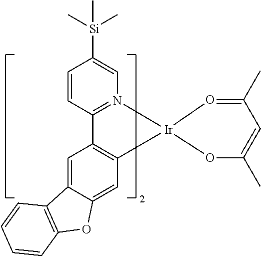

### First, Second, and Fourth Compounds Respectively Represented by Formulae 51, 61, and 91

Ring A₆₁ in Formula 61 may be represented by Formula 61A, and ring A₆₂ in Formula 61 may be represented by Formula 61B.

In Formula 61, ring A₆₁ may be represented by Formula 61A, and ring A₆₂ may be represented by Formula 61B. Ring A₆₁ may be condensed with a neighboring 5-membered ring and ring A₆₂, while sharing carbon therewith, and ring A₆₂ may be condensed with ring A₆₁ and a neighboring 6-membered ring, while sharing carbon therewith.

X₆₁ in Formula 61B may be N-[(L₆₂)ₐ₆₂-(R₆₂)_{b62}], S, O, S(═O), S(═O)₂, C(═O), C(R₆₃)(R₆₄), Si(R₆₃)(R₆₄), P(R₆₃), P(═O)(R₆₃), or C═N(R₆₃).

In Formula 61, X₇₁ may be C(R₇₁) or N, X₇₂ may be C(R₇₂) or N, X₇₃ may be C(R₇₃) or N, X₇₄ may be C(R₇₄) or N, X₇₅ may be C(R₇₅) or N, X₇₆ may be C(R₇₆) or N, X₇₇ may be C(R₇₇) or N, and X₇₈ may be C(R₇₈) or N.

In one or more embodiments, in Formula 61, X₇₁ may be C(R₇₁), X₇₂ may be C(R₇₂), X₇₃ may be C(R₇₃), X₇₄ may be C(R₇₄), X₇₅ may be C(R₇₅), X₇₆ may be C(R₇₆), X₇₇ may be C(R₇₇), and X₇₃ may be C(R₇₃).

L₅₁ to L₅₃, L₆₁, L₆₂, and L₉₁ to L₉₅ in Formulae 51, 61, 61B, and 91 may each independently be selected from a single bond, a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group.

In one or more embodiments, L₅₁ to L₅₃, L₆₁, L₆₂, and to L₉₅ in Formulae 51, 61, 61B, and 91 may each independently be selected from:

a single bond, a cyclopentylene group, a cyclohexylene group, a cyclopentenylene group, a cyclohexenylene group, a cycloheptenylene group, a phenylene group, a pentalenylene group, an indenylene group, a naphthylene group, an azulenylene group, a heptalenylene group, an indacenylene group, an acenaphthylene group, a benzoindenylene group, a fluorenylene group, a spiro-bifluorenylene group, a benzofluorenylene group, a dibenzofluorenylene group, a phenalenylene group, a phenanthrenylene group, an anthracenylene group, a fluoranthrenylene group, a triphenylenylene group, a pyrenylene group, a chrysenylene group, a naphthacenylene group, a picenylene group, a perylenylene group, a pentaphenylene group, a hexacenylene group, an imidazolylene group, a pyrazolylene group, a pyridinylene group, a pyrazinylene group, a pyrimidinylene group, a pyridazinylene group, a triazinylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzoxazolylene group, a benzimidazolylene group, a thiazolylene group, an isothiazolylene group, a benzothiazolylene group, an isoxazolylene group, an oxazolylene group, a triazolylene group, a tetrazolylene group, an oxadiazolylene group, an imidazopyrimidinylene group, an imidazopyridinylene group, a pyrrolylene group, a furanylene group, a thiophenylene group, a silolylene group, an isoindolylene group, an indolylene group, a benzofuranylene group, a benzothiophenylene group, a benzosilolylene group, a carbazolylene group, a dibenzofuranylene group, a dibenzothiophenylene group, a dibenzosilolylene group, a benzocarbazolylene group, a benzonaphthofuranylene group, a benzonaphthothiophenylene group, a benzonaphthosilolylene group, a pyridoindolylene group, a benzofuropyridinylene group, a benzothienopyridinylene group, a pyrimidoindolylene group, a benzofuropyrimidinylene group, a benzothienopyrimidinylene group, a phenoxazinylene group, a pyridobenzooxazinylene group, and a pyridobenzothiazinylene group; and

a cyclopentylene group, a cyclohexylene group, a cyclopentenylene group, a cyclohexenylene group, a cycloheptenylene group, a phenylene group, a pentalenylene group, an indenylene group, a naphthylene group, an azulenylene group, a heptalenylene group, an indacenylene group, an acenaphthylene group, a benzoindenylene group, a fluorenylene group, a spiro-bifluorenylene group, a benzofluorenylene group, a dibenzofluorenylene group, a phenalenylene group, a phenanthrenylene group, an anthracenylene group, a fluoranthrenylene group, a triphenylenylene group, a pyrenylene group, a chrysenylene group, a naphthacenylene group, a picenylene group, a perylenylene group, a pentaphenylene group, a hexacenylene group, an imidazolylene group, a pyrazolylene group, a pyridinylene group, a pyrazinylene group, a pyrimidinylene group, a pyridazinylene group, a triazinylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzoxazolylene group, a benzimidazolylene group, a thiazolylene group, an isothiazolylene group, a benzothiazolylene group, an isoxazolylene group, an oxazolylene group, a triazolylene group, a tetrazolylene group, an oxadiazolylene group, an imidazopyrimidinylene group, an imidazopyridinylene group, a pyrrolylene group, a furanylene group, a thiophenylene group, a silolylene group, an isoindolylene group, an indolylene group, a benzofuranylene group, a benzothiophenylene group, a benzosilolylene group, a carbazolylene group, a dibenzofuranylene group, a dibenzothiophenylene group, a dibenzosilolylene group, a benzocarbazolylene group, a benzonaphthofuranylene group, a benzonaphthothiophenylene group, a benzonaphthosilolylene group, a pyridoindolylene group, a benzofuropyridinylene group, a benzothienopyridinylene group, a pyrimidoindolylene group, a benzofuropyrimidinylene group, a benzothienopyrimidinylene group, a phenoxazinylene group, a pyridobenzooxazinylene group, and a pyridobenzothiazinylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a pyridinylphenyl group, a naphthyl group, an anthracenyl group, a pyrenyl group, a phenanthrenyl group, a fluorenyl group, a triphenylenyl group, a pyridinyl group, a phenylpyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, a carbazolyl group, a dibenzofuranyl group, a dibenzothiophenyl group, and —Si(Q₃₃)(Q₃₄)(Q₃₅),

wherein Q₃₃ to Q₃₅ may each independently be selected from a C₁-C₂₀ alkyl group, a C₁-C₂₀ alkoxy group, a phenyl group, a biphenyl group, a terphenyl group, a pyridinylphenyl group, a naphthyl group, a pyridinyl group, and a phenylpyridinyl group.

In one or more embodiments, L₅₁ to L₅₃, L₆₁, L₆₂, and L₉₁ to L₉₅ in Formulae 51, 61, 61B, and 91 may each independently be selected from a single bond and groups represented by Formulae 4-1 to 4-36, but embodiments are not limited thereto:

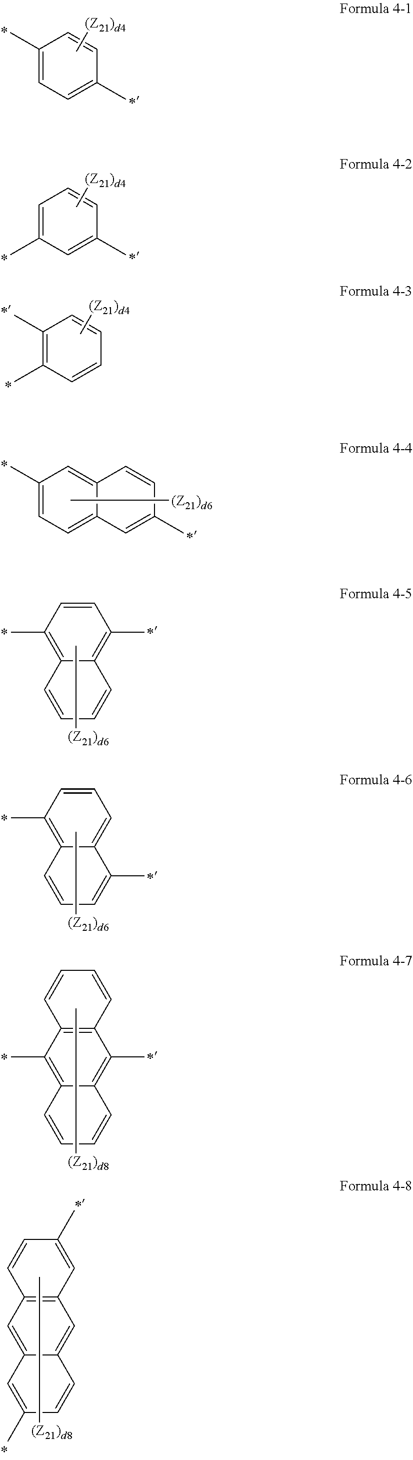

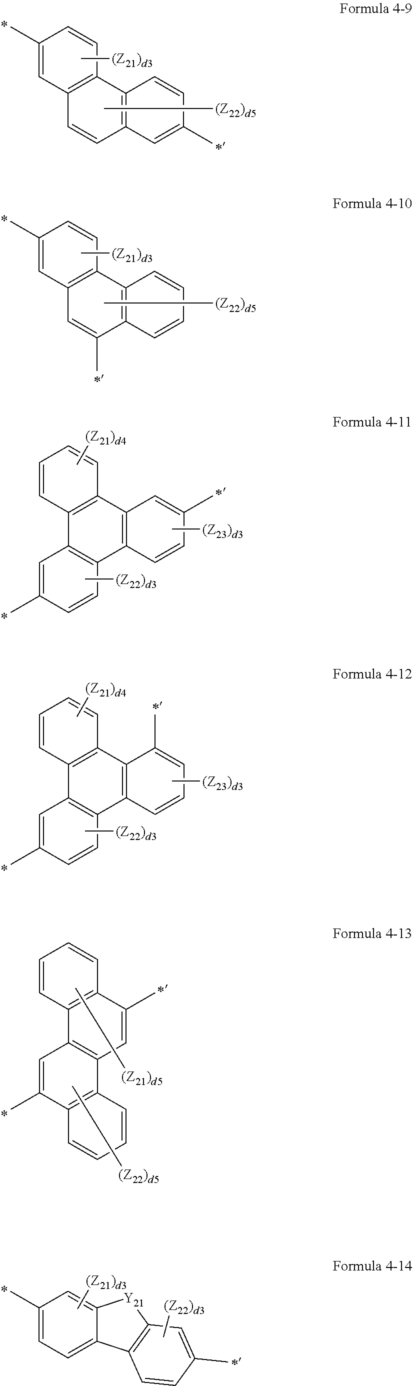

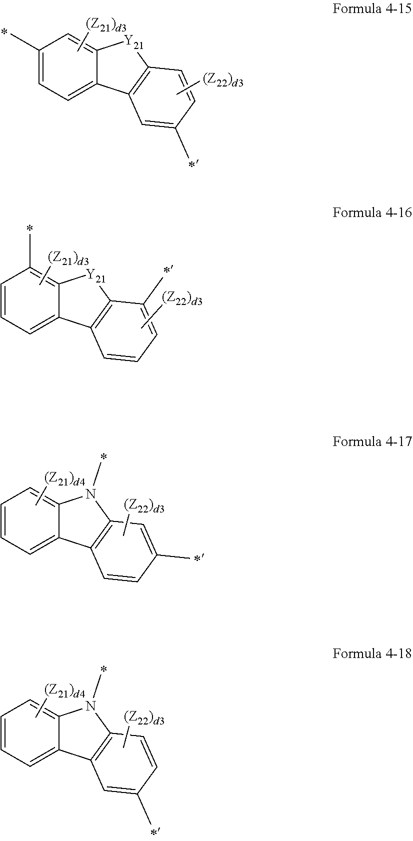

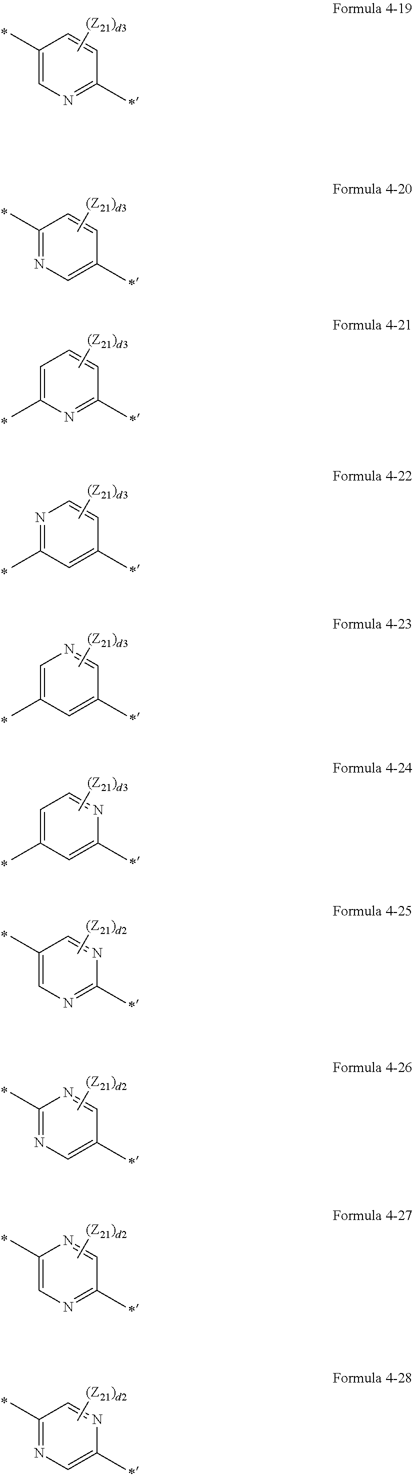

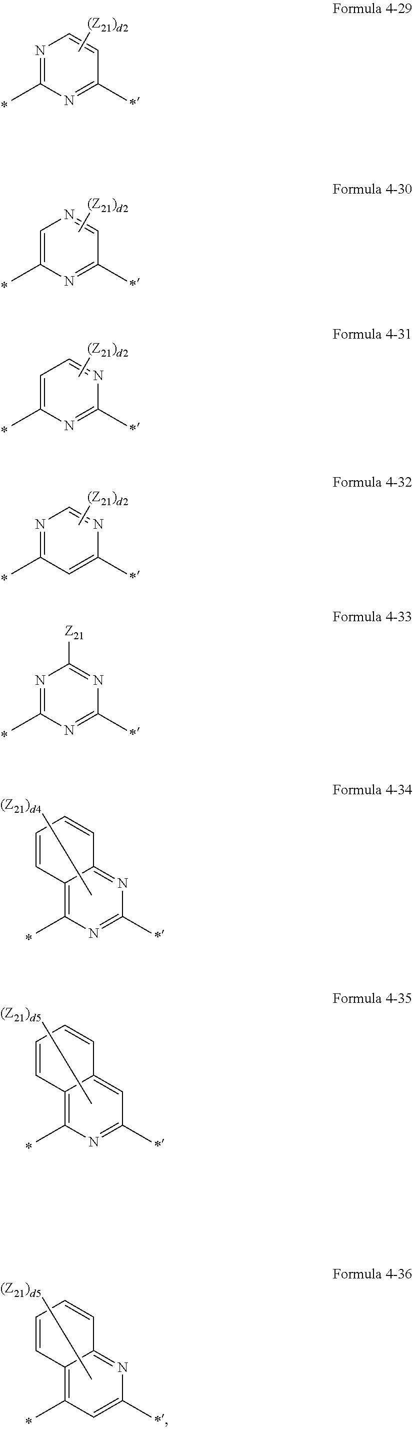

wherein, in Formulae 4-1 to 4-36,

Y₂₁ may be O, S, N(Z₂₃), C(Z₂₃)(Z₂₄), or Si(Z₂₃)(Z₂₄),

Z₂₁ to Z₂₄ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a pyridinylphenyl group, a naphthyl group, an anthracenyl group, a pyrenyl group, a phenanthrenyl group, a fluorenyl group, a triphenylenyl group, a pyridinyl group, a phenylpyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, a carbazolyl group, a dibenzofuranyl group, a dibenzothiophenyl group, and —Si(Q₃₃)(Q₃₄)(Q₃₅),

wherein Q₃₃ to Q₃₅ may each independently be selected from a C₁-C₂₀ alkyl group, a C₁-C₂₀ alkoxy group, a phenyl group, a biphenyl group, a terphenyl group, a pyridinylphenyl group, a naphthyl group, a pyridinyl group, and a phenylpyridinyl group,

d2 may be an integer selected from 0 to 2,

d3 may be an integer selected from 0 to 3,

d4 may be an integer selected from 0 to 4,

d5 may be an integer selected from 0 to 5,

d6 may be an integer selected from 0 to 6,

d8 may be an integer selected from 0 to 8, and

* and *′ each indicate a binding site to a neighboring atom.

a51 to a53, a61, a62, and a91 to a95 in Formulae 51, 61, 61B, and 91 respectively indicate the number of groups L₅₁ to L₅₃, L₆₁, L₆₂, and L₉₁ to L₉₅, and may each independently be an integer selected from 1 to 5.

In one or more embodiments, a51 to a53, a61, a62, and a91 to a95 in Formulae 51, 61, 61B, and 91 may each independently be 1, 2, or 3, or may each independently be 1 or 2, but embodiments are not limited thereto.

When a51 in Formula 51 is two or more, two or more groups L₅₁ may be identical to or different from each other. a52 to a53, a61, a62 and a91 to a95 are the same as described above in connection with a51.

R₅₁ to R₅₃, R₆₁ to R₆₄, R₇₁ to R₇₉, and R₉₁ to R₉₅ in Formulae 51, 61, 61A, 61B, and 91 may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁₁)(Q₁₂), —Si(Q₁₃)(Q₁₄)(Q₁₅), and —B(Q₁₆)(Q₁₇).

In one or more embodiments, L₅₁ to L₅₃, L₆₁, L₆₂, and to L₉₅ in Formulae 51, 61, 61B, and 91 may each independently be selected from:

a single bond, a phenylene group, a naphthylene group, a benzoindenylene group, a fluorenylene group, a spiro-bifluorenylene group, a benzofluorenylene group, a dibenzofluorenylene group, a phenanthrenylene group, an anthracenylene group, a fluoranthrenylene group, a triphenylenylene group, a pyrenylene group, a chrysenylene group, a pyridinylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a phenanthridinylene group, a phenanthrolinylene group, an imidazopyrimidinylene group, an imidazopyridinylene group, a pyrrolylene group, a furanylene group, a thiophenylene group, a silolylene group, an isoindolylene group, an indolylene group, a benzofuranylene group, a benzothiophenylene group, a benzosilolylene group, a carbazolylene group, a dibenzofuranylene group, a dibenzothiophenylene group, a dibenzosilolylene group, a benzocarbazolylene group, a benzonaphthofuranylene group, a benzonaphthothiophenylene group, and a benzonaphthosilolylene group; and

a single bond, a phenylene group, a naphthylene group, a benzoindenylene group, a fluorenylene group, a spiro-bifluorenylene group, a benzofluorenylene group, a dibenzofluorenylene group, a phenanthrenylene group, an anthracenylene group, a fluoranthrenylene group, a triphenylenylene group, a pyrenylene group, a chrysenylene group, a pyridinylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a phenanthridinylene group, a phenanthrolinylene group, an imidazopyrimidinylene group, an imidazopyridinylene group, a pyrrolylene group, a furanylene group, a thiophenylene group, a silolylene group, an isoindolylene group, an indolylene group, a benzofuranylene group, a benzothiophenylene group, a benzosilolylene group, a carbazolylene group, a dibenzofuranylene group, a dibenzothiophenylene group, a dibenzosilolylene group, a benzocarbazolylene group, a benzonaphthofuranylene group, a benzonaphthothiophenylene group, and a benzonaphthosilolylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a pyridinylphenyl group, a naphthyl group, an anthracenyl group, a pyrenyl group, a phenanthrenyl group, a fluorenyl group, a triphenylenyl group, a pyridinyl group, a phenylpyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, a carbazolyl group, a dibenzofuranyl group, a dibenzothiophenyl group, and —Si(Q₃₃)(Q₃₄)(Q₃₅),

but embodiments are not limited thereto.

R₅₁ to R₅₃, R₆₁ to R₆₄, R₇₁ to R₇₉, and R₉₁ to R₉₅ in Formulae 51, 61, 61A, 61B, and 91 may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;

a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a benzoindenyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, an imidazolyl group, a pyrazolyl group, a pyridinyl group, a pyrazinyl group, a pyrimidinyl group, a pyridazinyl group, a triazinyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a thiazolyl group, an isothiazolyl group, a benzothiazolyl group, an isoxazolyl group, an oxazolyl group, a triazolyl group, a tetrazolyl group, an oxadiazolyl group, an imidazopyrimidinyl group, an imidazopyridinyl group, a pyrrolyl group, a furanyl group, a thiophenyl group, a silolyl group, an isoindolyl group, an indolyl group, a benzofuranyl group, a benzothiophenyl group, a benzosilolyl group, a carbazolyl group, a dibenzofuranyl group, a dibenzothiophenyl group, a dibenzosilolyl group, a benzocarbazolyl group, a benzonaphthofuranyl group, a benzonaphthothiophenyl group, a benzonaphthosilolyl group, a pyridoindolyl group, a benzofuropyridinyl group, a benzothienopyridinyl group, a pyrimidoindolyl group, a benzofuropyrimidinyl group, a benzothienopyrimidinyl group, a phenoxazinyl group, a pyridobenzooxazinyl group, and a pyridobenzothiazinyl group;

a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a benzoindenyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, an imidazolyl group, a pyrazolyl group, a pyridinyl group, a pyrazinyl group, a pyrimidinyl group, a pyridazinyl group, a triazinyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a thiazolyl group, an isothiazolyl group, a benzothiazolyl group, an isoxazolyl group, an oxazolyl group, a triazolyl group, a tetrazolyl group, an oxadiazolyl group, an imidazopyrimidinyl group, an imidazopyridinyl group, a pyrrolyl group, a furanyl group, a thiophenyl group, a silolyl group, an isoindolyl group, an indolyl group, a benzofuranyl group, a benzothiophenyl group, a benzosilolyl group, a carbazolyl group, a dibenzofuranyl group, a dibenzothiophenyl group, a dibenzosilolyl group, a benzocarbazolyl group, a benzonaphthofuranyl group, a benzonaphthothiophenyl group, a benzonaphthosilolyl group, a pyridoindolyl group, a benzofuropyridinyl group, a benzothienopyridinyl group, a pyrimidoindolyl group, a benzofuropyrimidinyl group, a benzothienopyrimidinyl group, a phenoxazinyl group, a pyridobenzooxazinyl group and a pyridobenzothiazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a pyridinylphenyl group, a naphthyl group, an anthracenyl group, a pyrenyl group, a phenanthrenyl group, a fluorenyl group, a triphenylenyl group, a pyridinyl group, a phenylpyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, a carbazolyl group, a dibenzofuranyl group, a dibenzothiophenyl group, and —Si(Q₃₃)(Q₃₄)(Q₃₅); and

—Si(Q₁₃)(Q₁₄)(Q₁₅),

wherein Q₁₃ to Q₁₅ and Q₃₃ to Q₃₅ may each independently be selected from a C₁-C₂₀ alkyl group, a C₁-C₂₀ alkoxy group, a phenyl group, a biphenyl group, a terphenyl group, a pyridinylphenyl group, a naphthyl group, a pyridinyl group, and a phenylpyridinyl group.

In one or more embodiments, R₅₁ to R₅₃, R₆₁ to R₆₄, R₇₁ to R₇₉, and R₉₁ to R₉₅ in Formulae 51, 61, 61A, 61B, and 91 may each independently be selected from groups represented by Formulae 5-1 to 5-15, 6-1 to 6-6, and 7-1 to 7-50, but embodiments are not limited thereto:

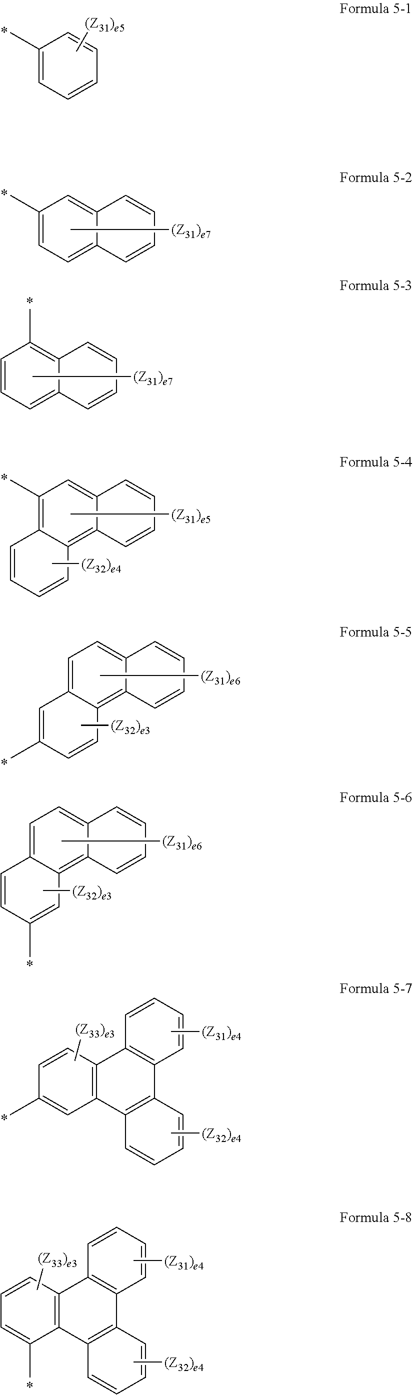

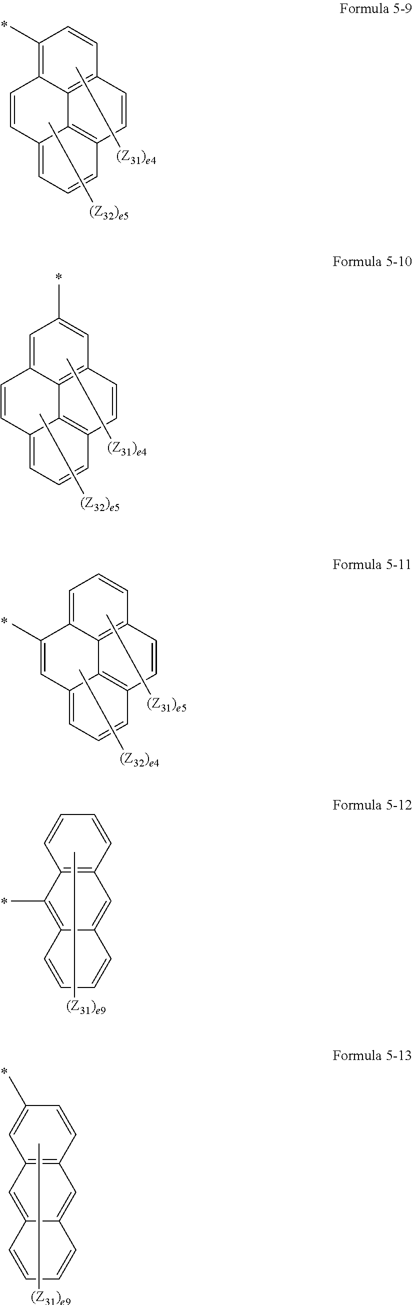

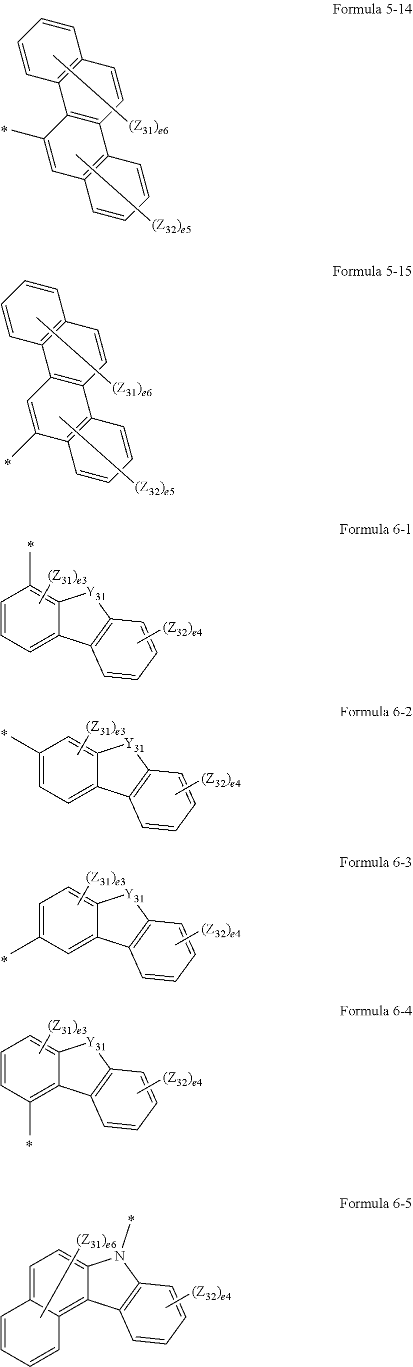

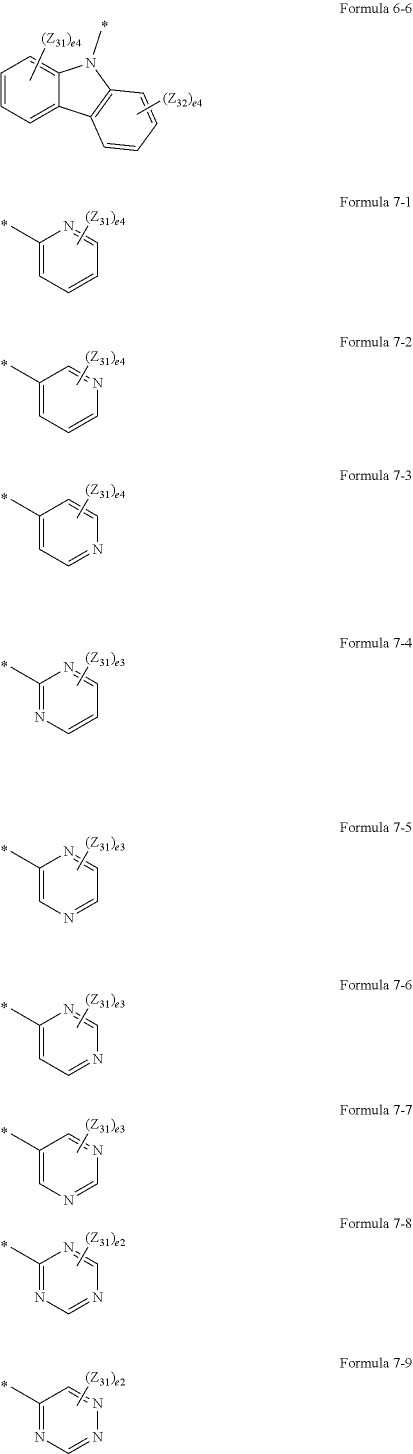

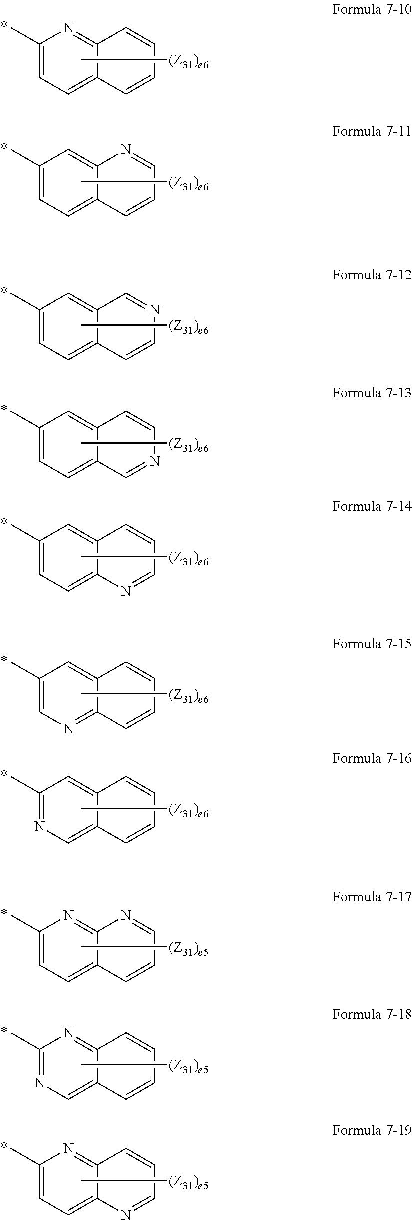

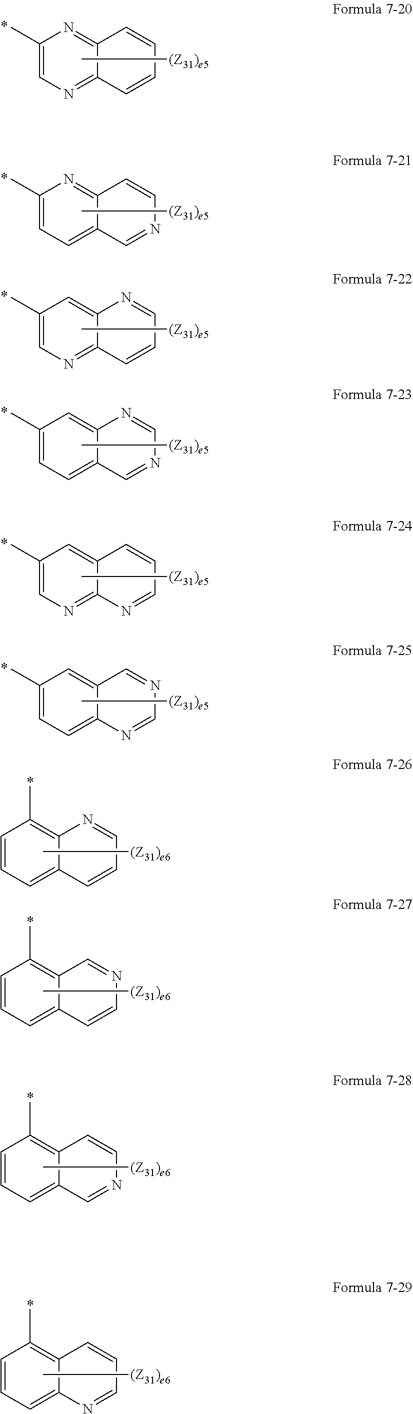

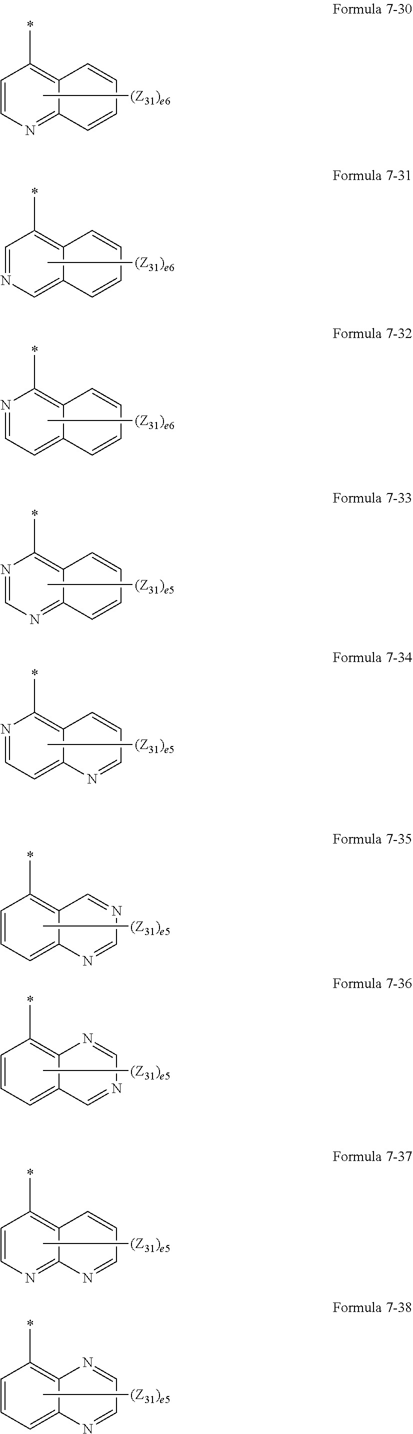

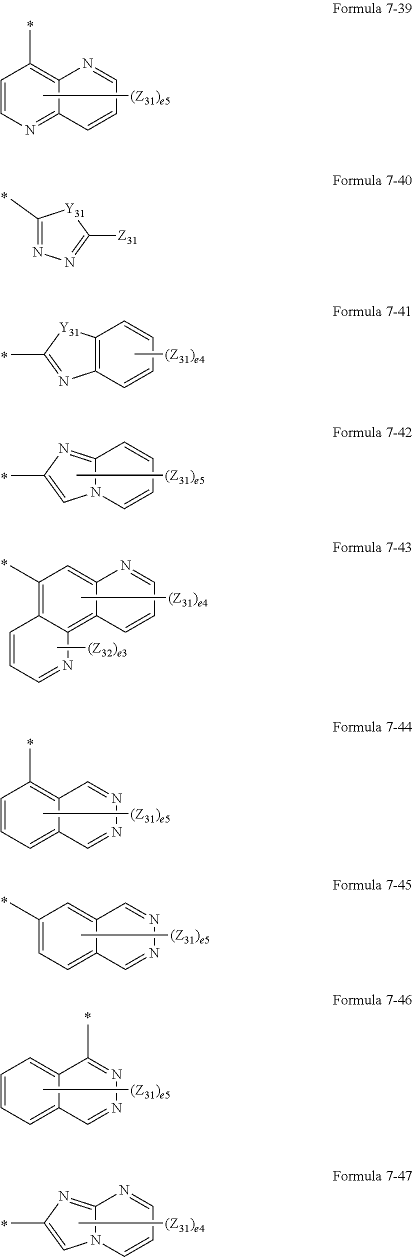

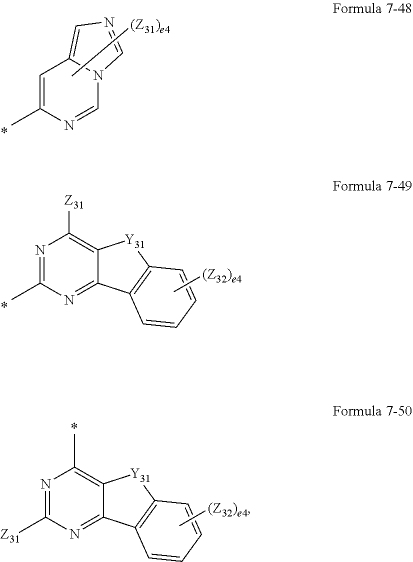

wherein, in Formulae 5-1 to 5-15, 6-1 to 6-6, and 7-1 to 7-50,

Y₃₁ may be O, S, N(Z₃₃), C(Z₃₃)(Z₃₄), or Si(Z₃₃)(Z₃₄),

Z₃₁ to Z₃₄ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a pyridinylphenyl group, a naphthyl group, an anthracenyl group, a pyrenyl group, a phenanthrenyl group, a fluorenyl group, a triphenylenyl group, a pyridinyl group, a phenylpyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, a carbazolyl group, a dibenzofuranyl group, a dibenzothiophenyl group, and —Si(Q₃₃)(Q₃₄)(Q₃₅),

wherein Q₃₃ to Q₃₅ may each independently be selected from a C₁-C₂₀ alkyl group, a C₁-C₂₀ alkoxy group, a phenyl group, a biphenyl group, a terphenyl group, a pyridinylphenyl group, a naphthyl group, a pyridinyl group, and a phenylpyridinyl group,

e2 may be an integer selected from 0 to 2,

e3 may be an integer selected from 0 to 3,

e4 may be an integer selected from 0 to 4,

e5 may be an integer selected from 0 to 5,

e6 may be an integer selected from 0 to 6,

e7 may be an integer selected from 0 to 7,

e9 may be an integer selected from 0 to 9, and

* indicates a binding site to a neighboring atom.

b61, b62, and b79 in Formulae 61A and 61B respectively indicate the number of groups R₆₁, R₆₂, and R₇₉, and may each independently be an integer selected from 0 to 3. In one or more embodiments, b61 and b62 may be 1 or 2, but embodiments are not limited thereto.

In one or more embodiments, in Formula 51,

L₅₁ to L₅₃ may each independently be selected from a single bond and groups represented by Formulae 4-1 to 4-18,

a51 to a53 may each independently be 1 or 2,

R₅₁ to R₅₃ may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group; and

groups represented by Formulae 5-1 to 5-15 and 6-1 to 6-6, and

at least one selected from R₅₁ to R₅₃ may be selected from groups represented by Formulae 6-1 to 6-6.

In one or more embodiments, X₆₁ in Formula 61 may be N-[(L₆₂)ₐ₆₂-(R₆₂)_{b62}].

In one or more embodiments, in Formula 61,

X₆₁ may be N-[(L₆₂)ₐ₆₂-(R₆₂)_{b62}],

at least one selected from L₆₁ and L₆₂ may be selected from groups represented by Formulae 4-19 to 4-36,

a61 and a62 may each independently be 1 or 2,

at least one selected from R₆₁ and R₆₂ may be selected from groups represented by Formulae 7-1 to 7-50,

b61 and b62 may be 1, but embodiments are not limited thereto.

In one or more embodiments, in Formula 61,

X₆₁ may be N-[(L₆₂)ₐ₆₂-(R₆₂)_{b62}],

at least one selected from R₆₁ and R₆₂ may be selected from groups represented by Formulae 6-1 to 6-6, and

b61 and b62 may be 1, but embodiments are not limited thereto.

At least one selected from to L₉₅ and R₉₁ to R₉₅ in Formula 91 may include a carbazole ring or a triphenylene ring. That is, the fourth compound represented by Formula 91 essentially includes at least one carbazole ring and at least one triphenylene ring.

In one or more embodiments,

the first compound may be represented by one selected from Formulae 51-1 to 51-3,

the second compound may be represented by one selected from Formulae 61-1 to 61-6,

the fourth compound may be represented by one selected from Formulae 91-1 to 91-14, but embodiments are not limited thereto:

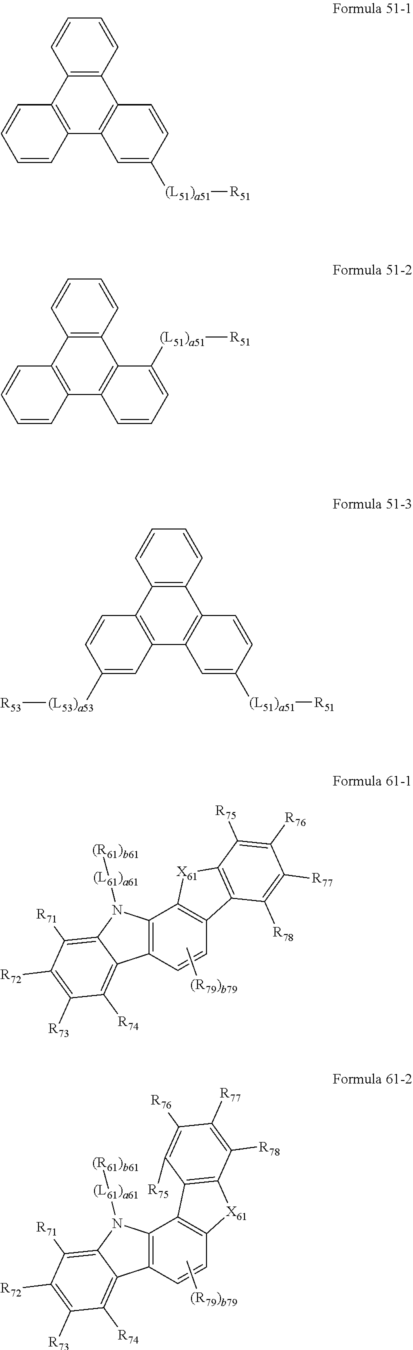

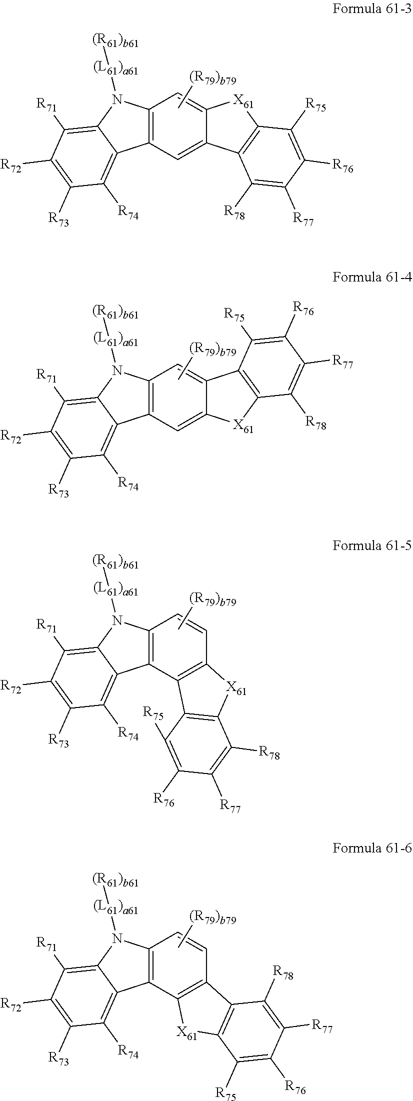

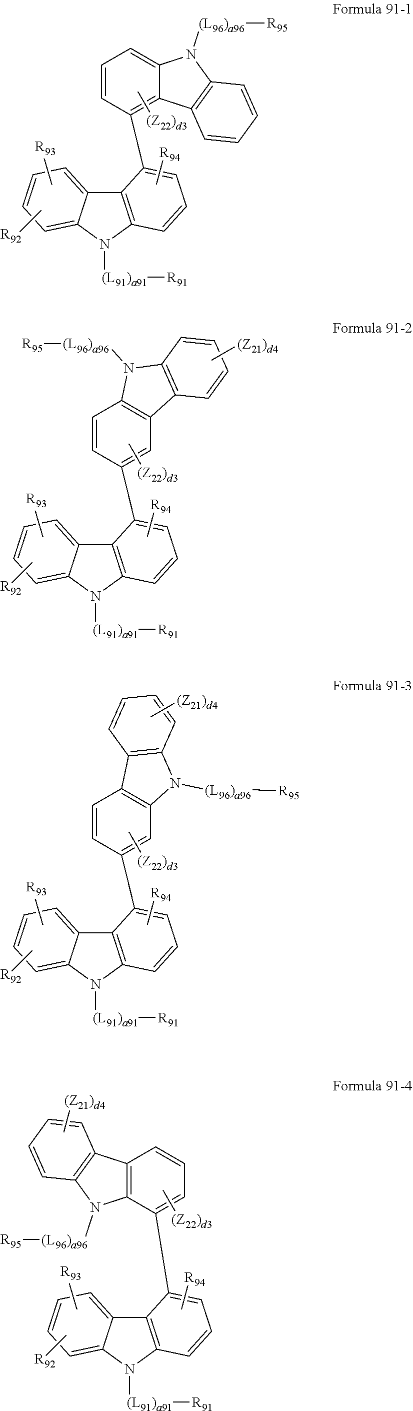

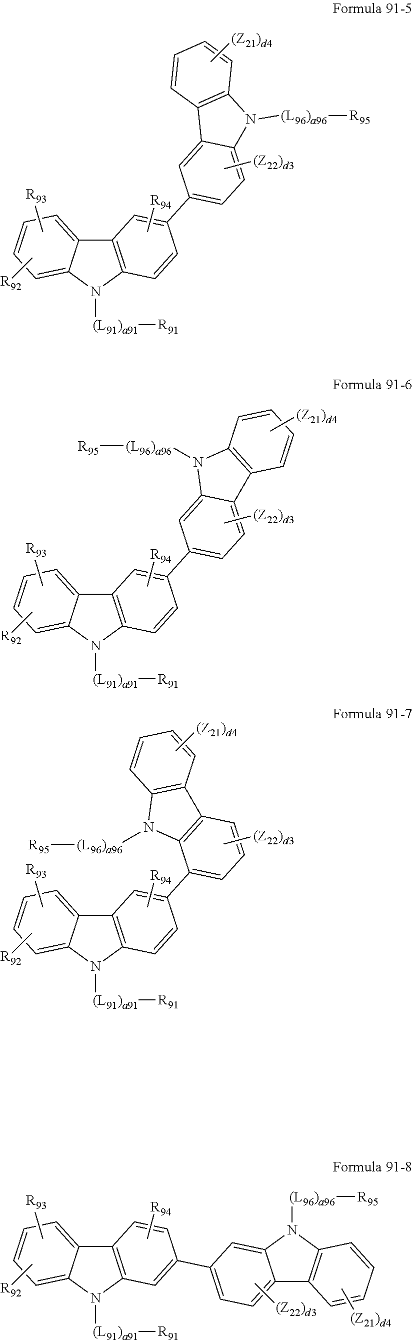

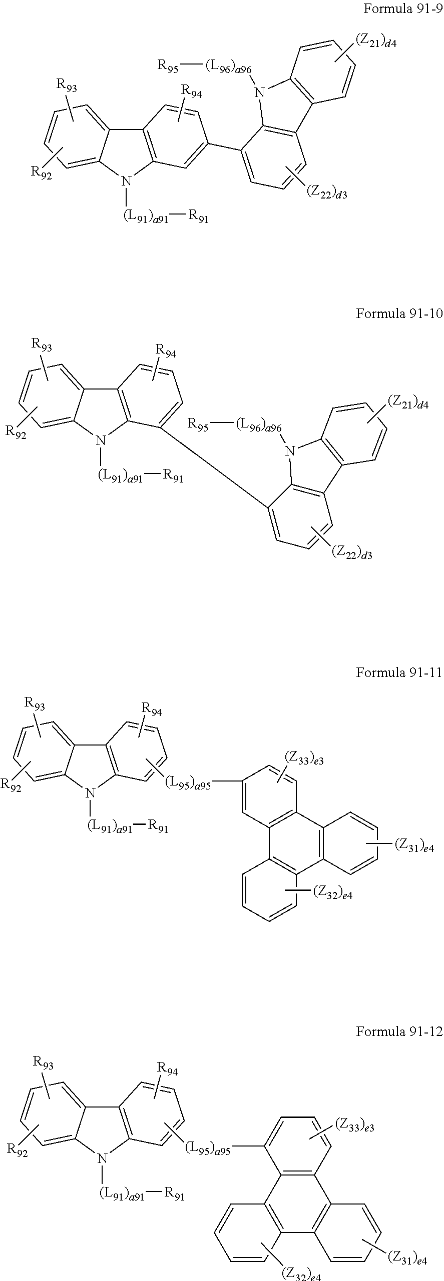

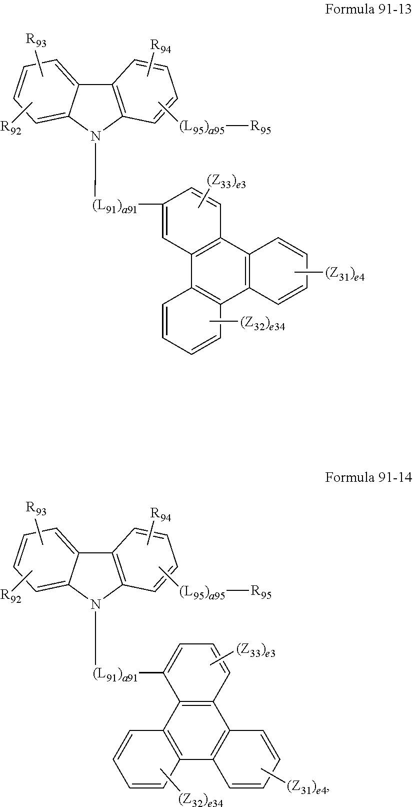

wherein L₅₁, L₅₃, a51, a53, R₅₁, and R₅₃ in Formulae 51-1 to 51-3 are the same as described above,

X₆₁, L₆₁, a61, R₆₁, b61, R₇₁ to R₇₉, and b79 in Formulae 61-1 to 61-6 are the same as described above,

in Formulae 91-1 to 91-14,

L₉₁, a91, and R₉₁ to R₉₅ are the same as described above,

L₉₆ is the same as described above in connection with L₉₁,

a96 may be 1 or 2,

Z₃₁ to Z₃₃ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a pyridinylphenyl group, a naphthyl group, an anthracenyl group, a pyrenyl group, a phenanthrenyl group, a fluorenyl group, a triphenylenyl group, a pyridinyl group, a phenylpyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, a carbazolyl group, a dibenzofuranyl group, a dibenzothiophenyl group, and —Si(Q₃₃)(Q₃₄)(Q₃₅),

wherein Q₃₃ to Q₃₅ may each independently be selected from a C₁-C₂₀ alkyl group, a C₁-C₂₀ alkoxy group, a phenyl group, a biphenyl group, a terphenyl group, a pyridinylphenyl group, a naphthyl group, a pyridinyl group, and a phenylpyridinyl group,

e3 may be an integer selected from 0 to 3, and

e4 may be an integer selected from 0 to 4.

### Third Compound Represented by Formula 81

In Formula 81, Z₁₁ may be N or C(Rₐ), Z₁₂ may be N or C(R_{b}), Z₁₁ may be N or C(R_{c}), Z₁₄ may be N or C(R_{d}), Z₁₅ may be N or C(Rₑ), Z₁₀ may be N or C(R_{f}), Z₁₇ may be N or C(R_{g}), Z₁₈ may be N or C(Rₕ), Z₁₉ may be N or C(Rᵢ), and at least one selected from Z₁₁ to Z₁₉ may be N.

Accordingly, since the third compound easily accepts electrons when an electric field is applied thereto, an organic light-emitting device including the third compound may have a low driving voltage. Since the third compound also includes a triphenylene core, the third compound may have a bipolar structure. Therefore, in the organic light-emitting device including the third compound represented by Formula 81, a flow of hole and electron may be balanced. Thus, the organic light-emitting device including the third compound may have high efficiency.

R₈₁ to R₉₀ and Rₐ to Rᵢ in Formula 81 may each independently be selected from hydrogen, deuterium, a substituted or unsubstituted C₁-C₁₀ alkyl group, and a substituted or unsubstituted C₆-C₁₂ aryl group.

In one or more embodiments, R₈₁ to R₉₀ and Rₐ to Rᵢ in Formula 81 may each independently be selected from hydrogen, deuterium, a C₁-C₁₀ alkyl group, and groups represented by Formulae 5-1 to 5-15, but embodiments are not limited thereto.

The number of 6-membered rings substituted with a triphenylene core in Formula 81 may be six or less,

L₈₁ in Formula 81 may be selected from a substituted or unsubstituted phenylene group, a substituted or unsubstituted biphenylene group, and a substituted or unsubstituted terphenylene group,

a81 to a83 in Formula 81 may each independently be 0 or 1, and

in Formula 81, a81+a82+a83≥1.

The 6-membered rings substituted with the triphenylene core in Formula 81 may refer to all 6-membered rings directly or indirectly linked to the triphenylene core and may include a 6-membered ring including a carbon atom, a nitrogen atom, or any combinations thereof.

The third compound may be represented by Formula 81-1 or 81-2:

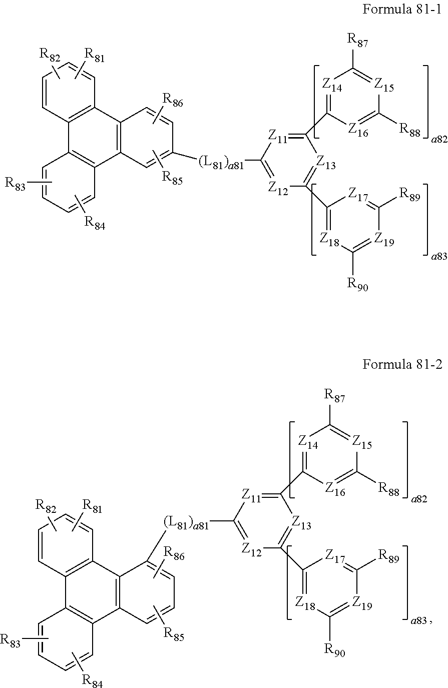

wherein various substituents in Formulae 81-1 and 81-2 are the same as described above.

The third compound represented by Formula 81 may have at least one kink structure.

The kink structure refers to a structure in which two linking portions that link three rings to one another do not form a linear structure with each other. For example, in the case of a phenylene group, an o-phenylene group and an m-phenylene group, in which linking portions do not form a linear structure, have the kink structure. A p-phenylene group, in which the linking portions form a linear structure, does not have a kink structure.

The kink structure in Formula 81 may be formed around a linking group (L₈₁) and/or arylene group/heteroarylene group.

In one or more embodiments, when a81 of Formula 81 is zero, that is, when *-(L₈₁)ₐ₈₁-*′ is a single bond, the kink structure may be formed around an arylene group/heteroarylene group.

In one or more embodiments, when a81 of Formula 81A is 1, the kink structure may be formed around a linking group (L₈₁) in Formula 81. In one or more embodiments, L₈₁ in Formula 81 may be a substituted or unsubstituted phenylene group having a kink structure, a substituted or unsubstituted biphenylene group having a kink structure, or a substituted or unsubstituted terphenylene group having a kink structure. In one or more embodiments, L₈₁ in Formula 81 may be selected from groups represented by Formulae 8-1 to 8-36:

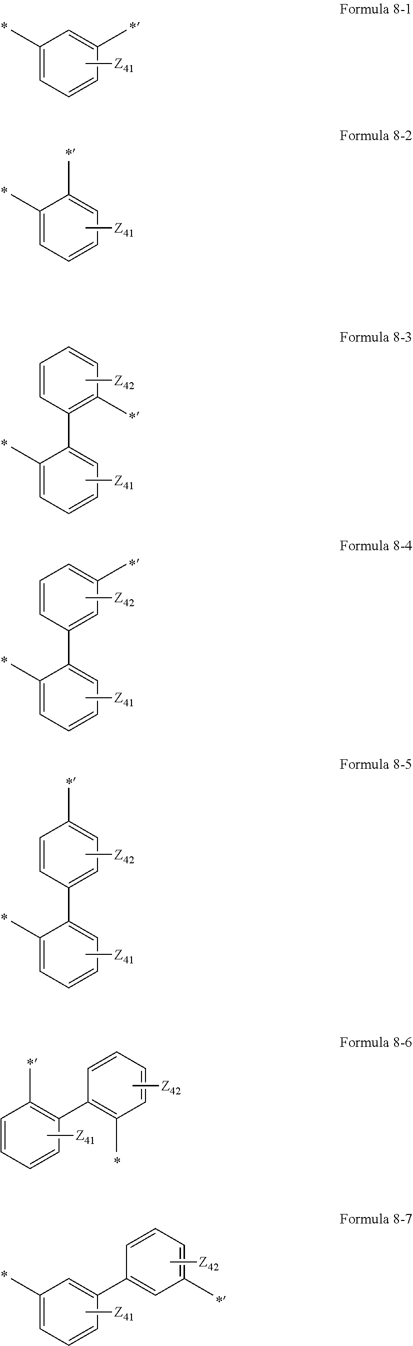

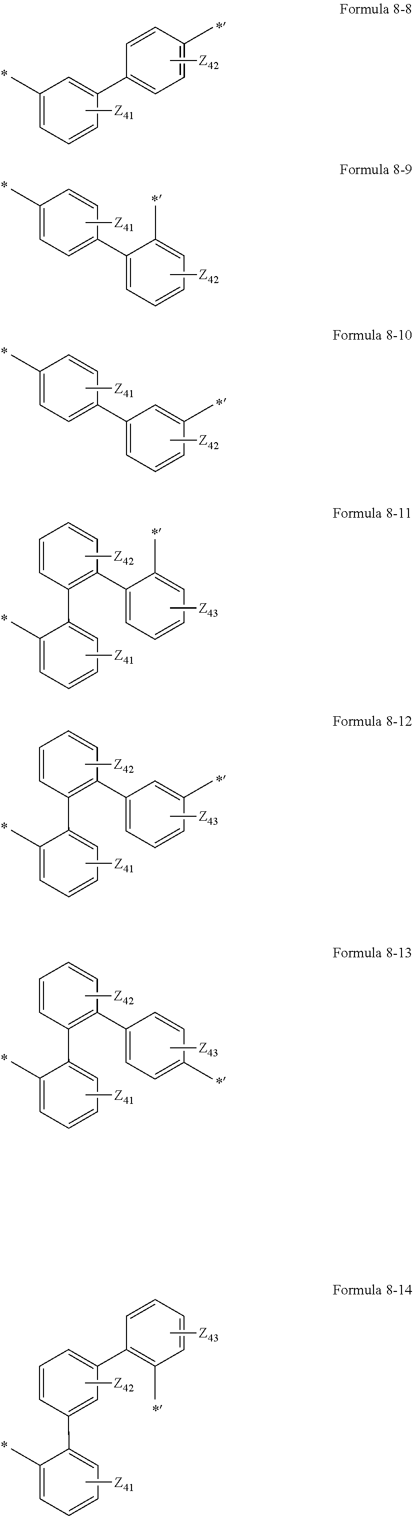

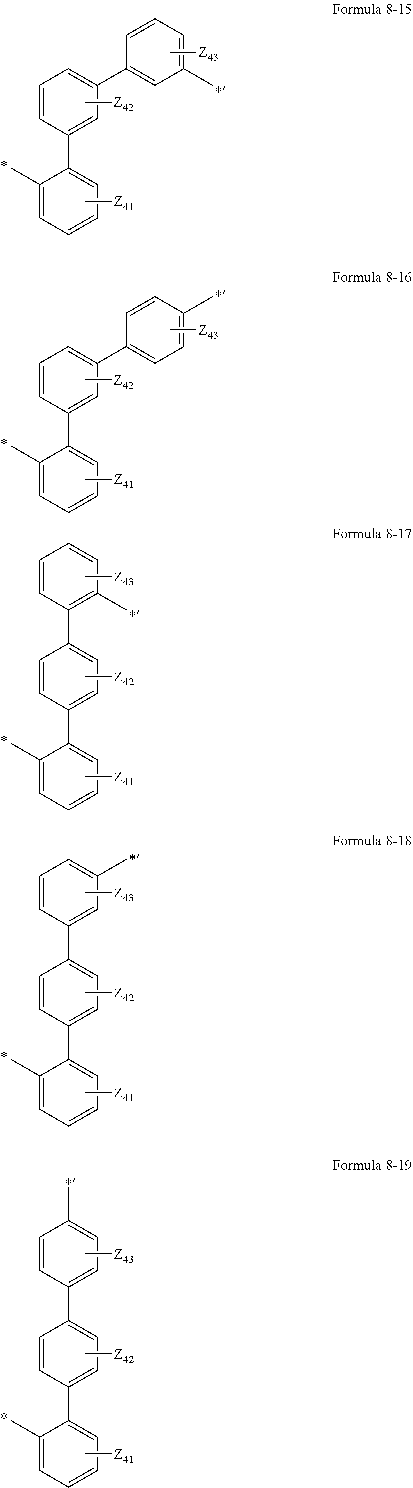

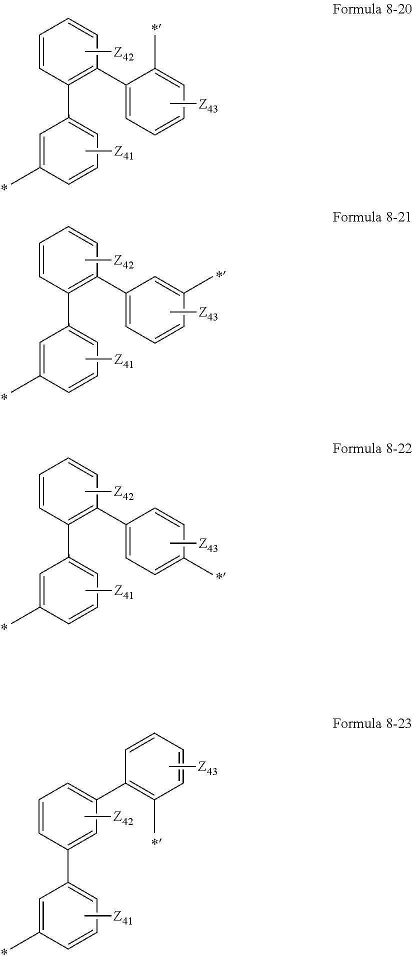

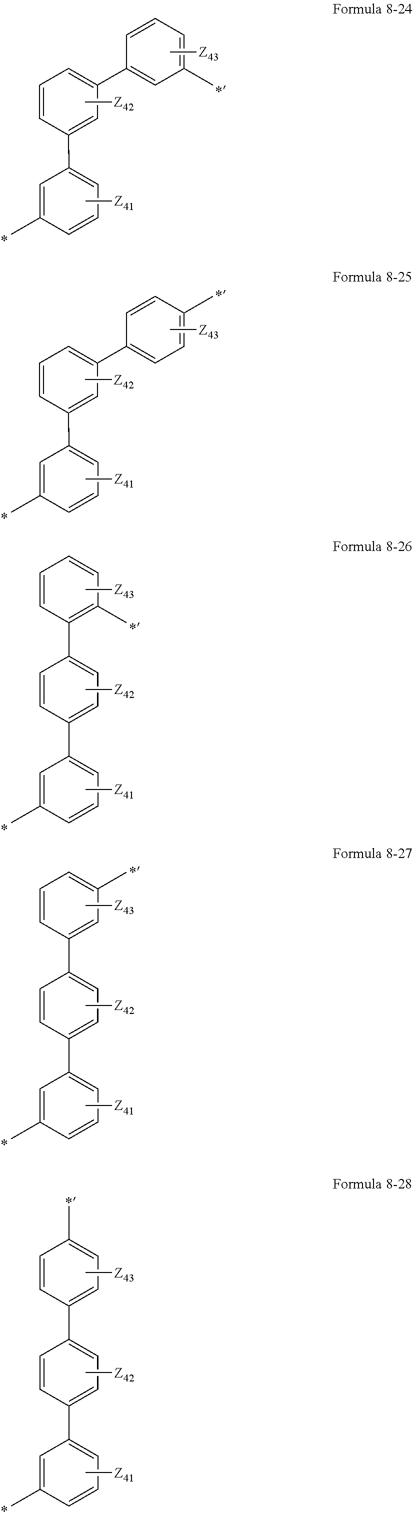

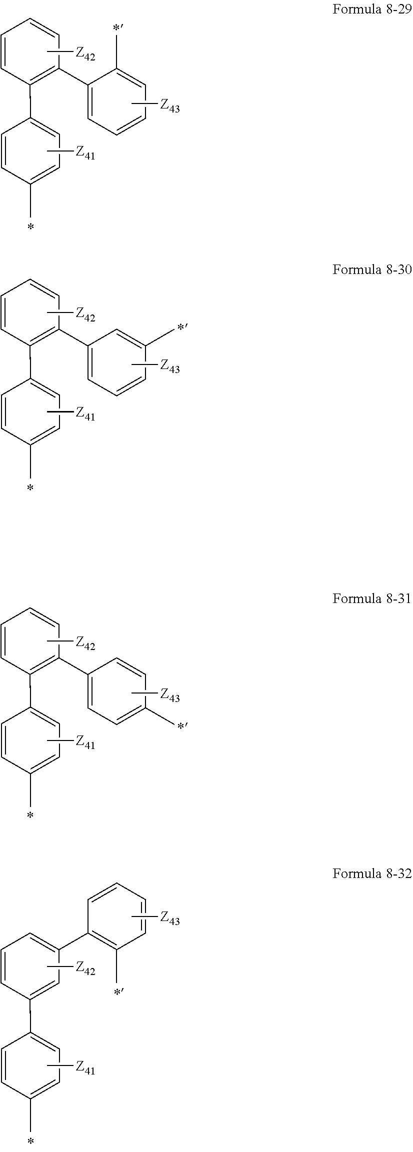

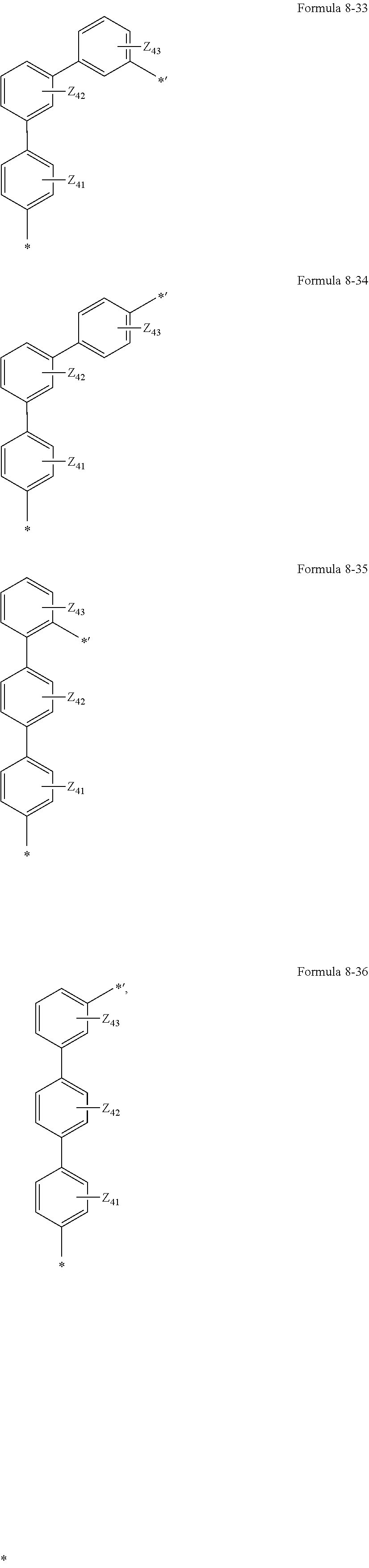

wherein Z₄₁ to Z₄₃ in Formulae 8-1 to 8-36 are the same as described above in connection with R₈₁ in Formula 81, and * and *′ each indicate a binding site to a neighboring atom.

Formula 81 may have at least two kink structures. In one or more embodiments, Formula 81 may have at least two to four kink structures.

Since the third compound represented by Formula 81 has the kink structure as described above, it is possible to appropriately localize charges and effectively control a flow of a conjugated system. Thus, an organic light-emitting device including the third compound represented by Formula 81 may have a long lifespan.

Also, in the third compound represented by Formula 81, stacking between molecules may be effectively prevented. Accordingly, in the case of forming a film by using the third compound represented by Formula 81, it is possible to improve process stability and reduce a deposition temperature. The stacking prevention effect may be further improved when a81 of Formula 81 is 1.

### Specific Examples of First to Fourth Compounds

The first compound may be selected from Compounds A1 to A18,

the second compound may be selected from Compounds B1 to B30,

the third compound may be selected from Compounds A-1 to A-125,

the fourth compound may be selected from Compounds B-10 to B-161, C-10 to C-33, and D-10 to D-29, but embodiments are not limited thereto:

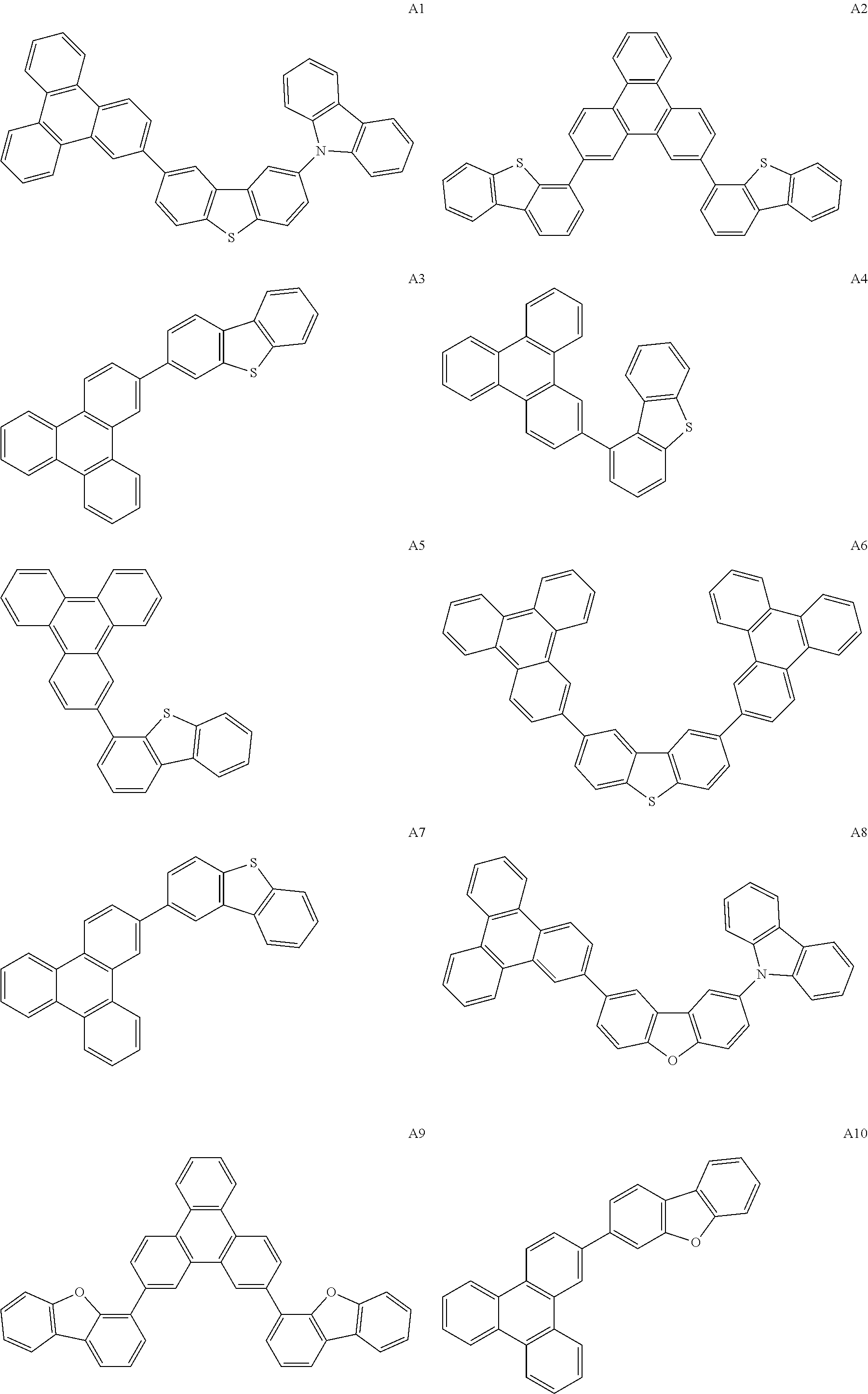

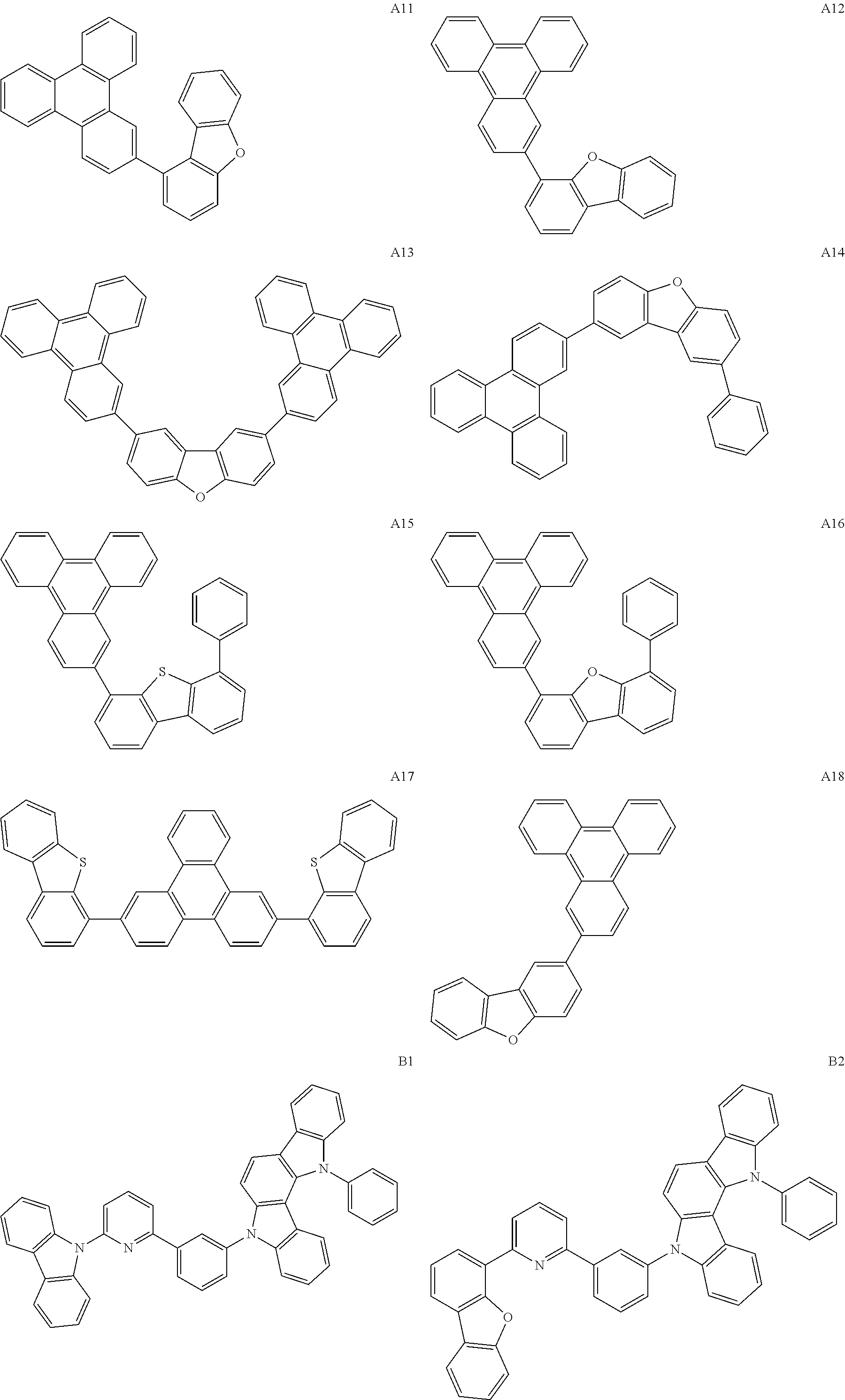

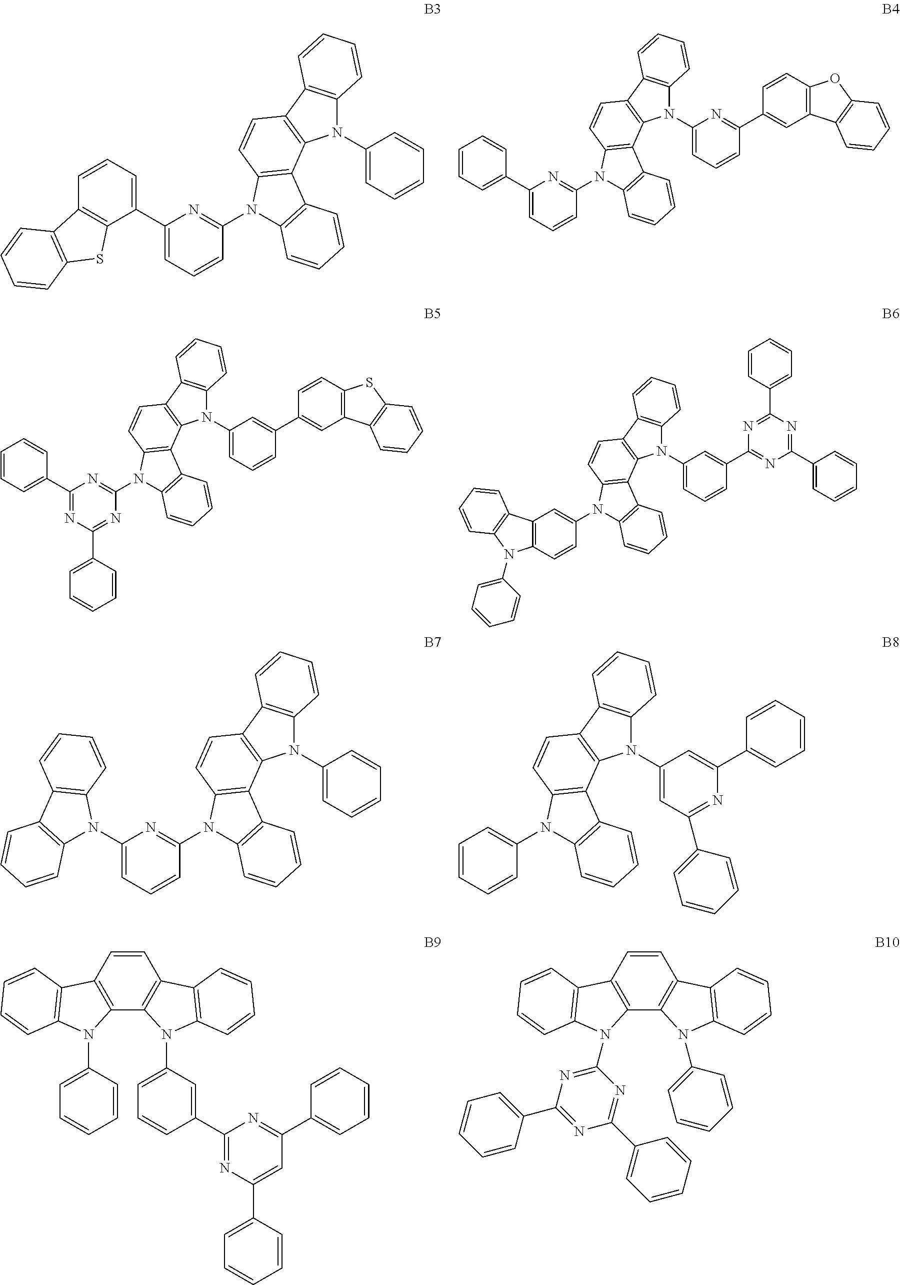

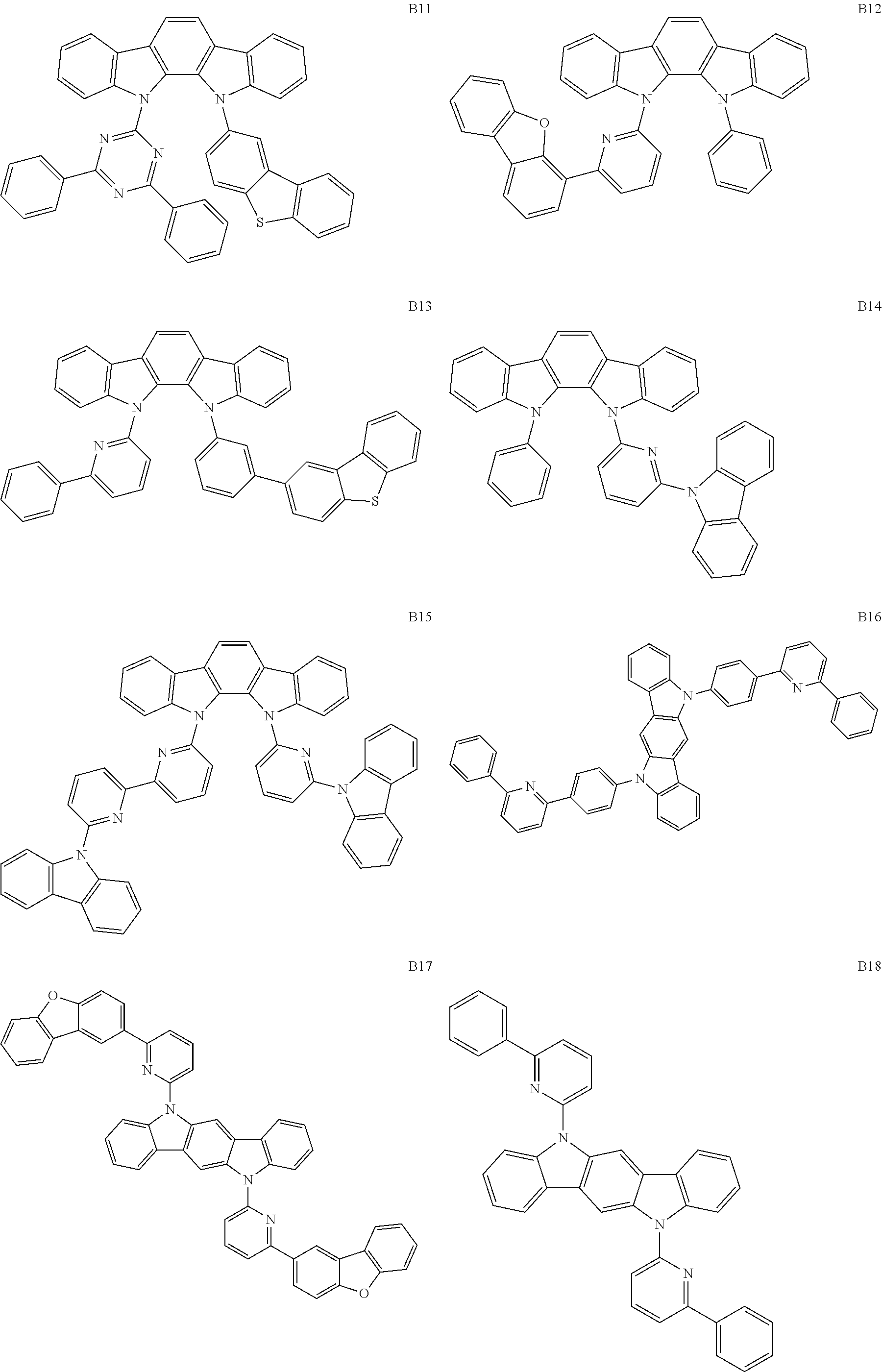

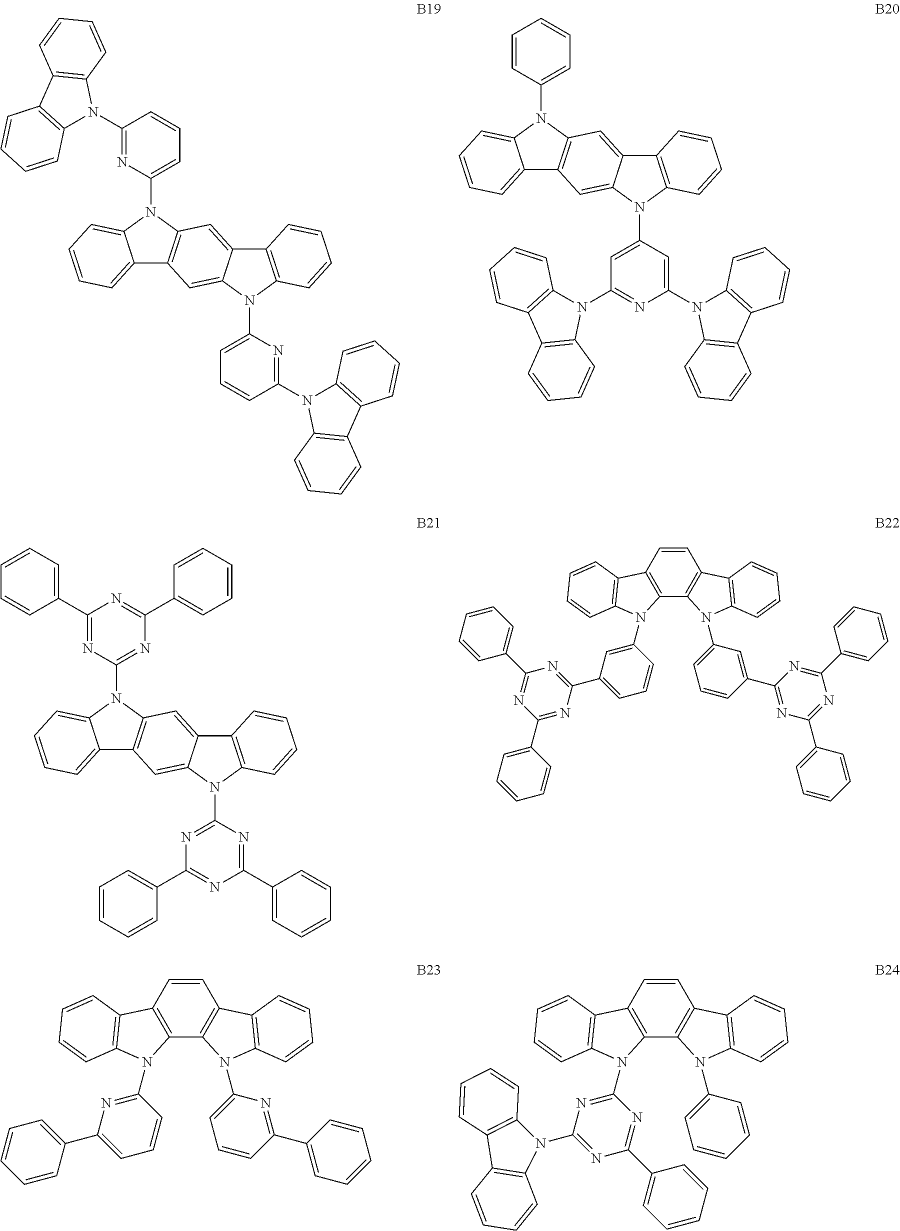

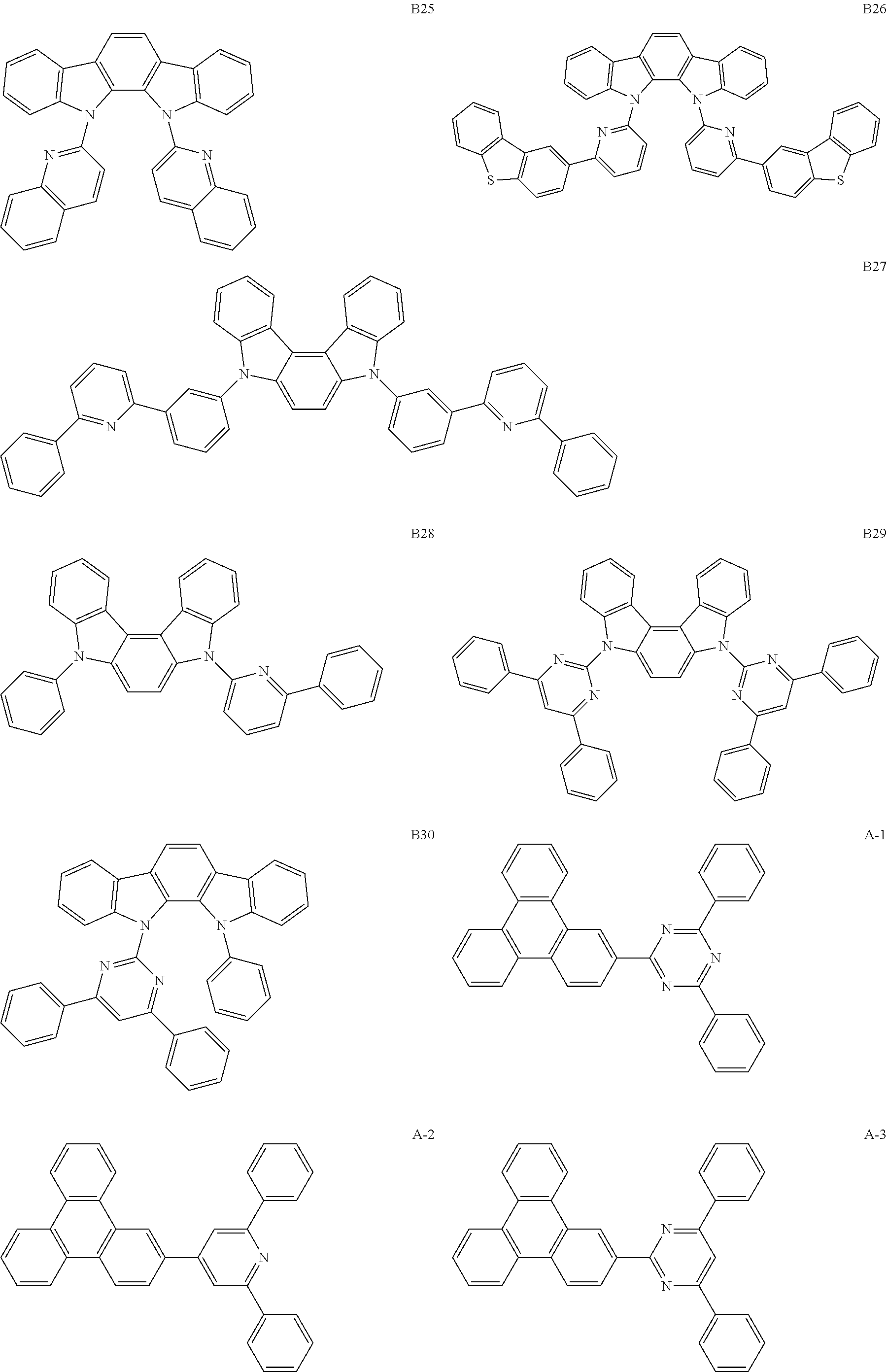

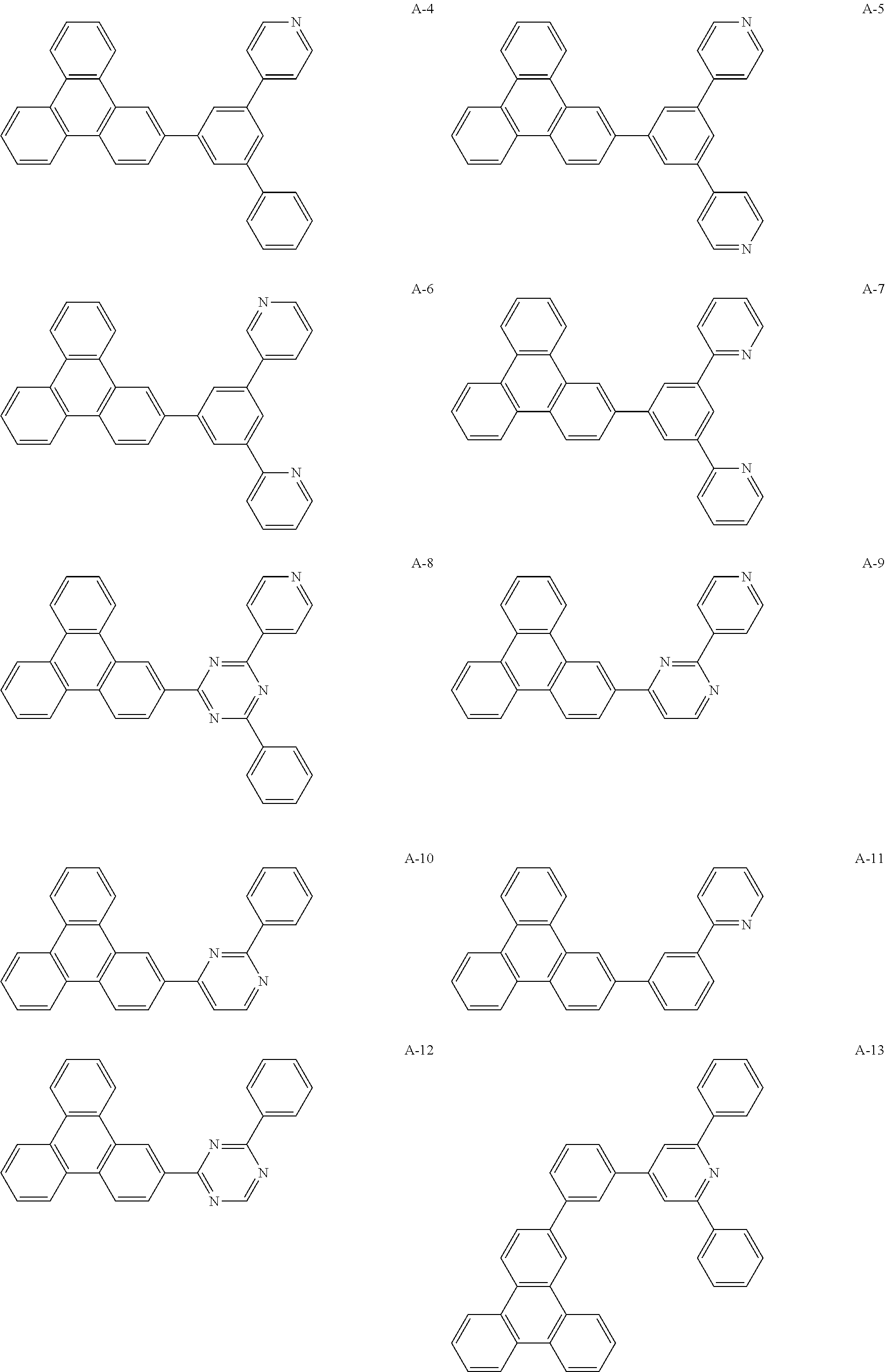

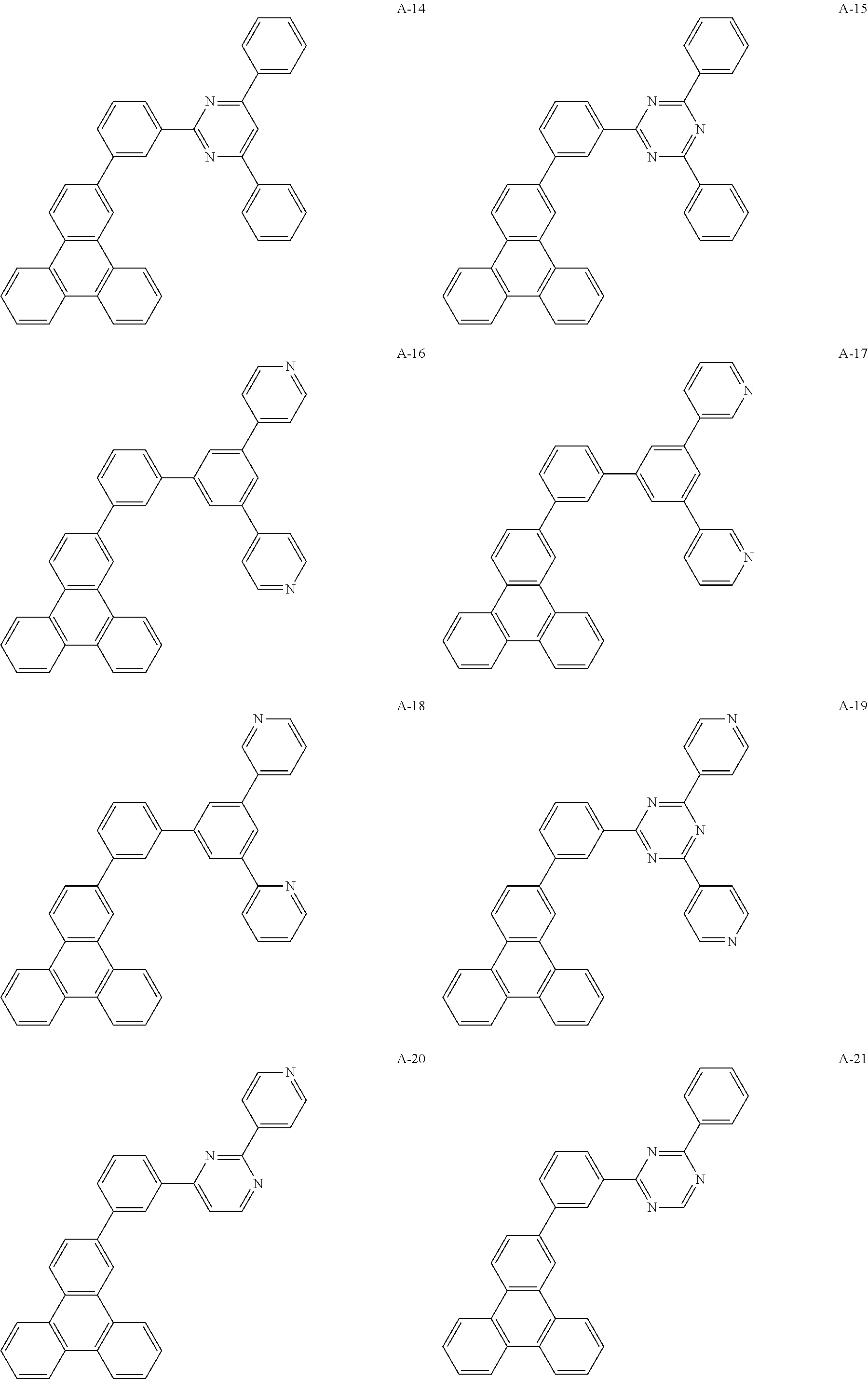

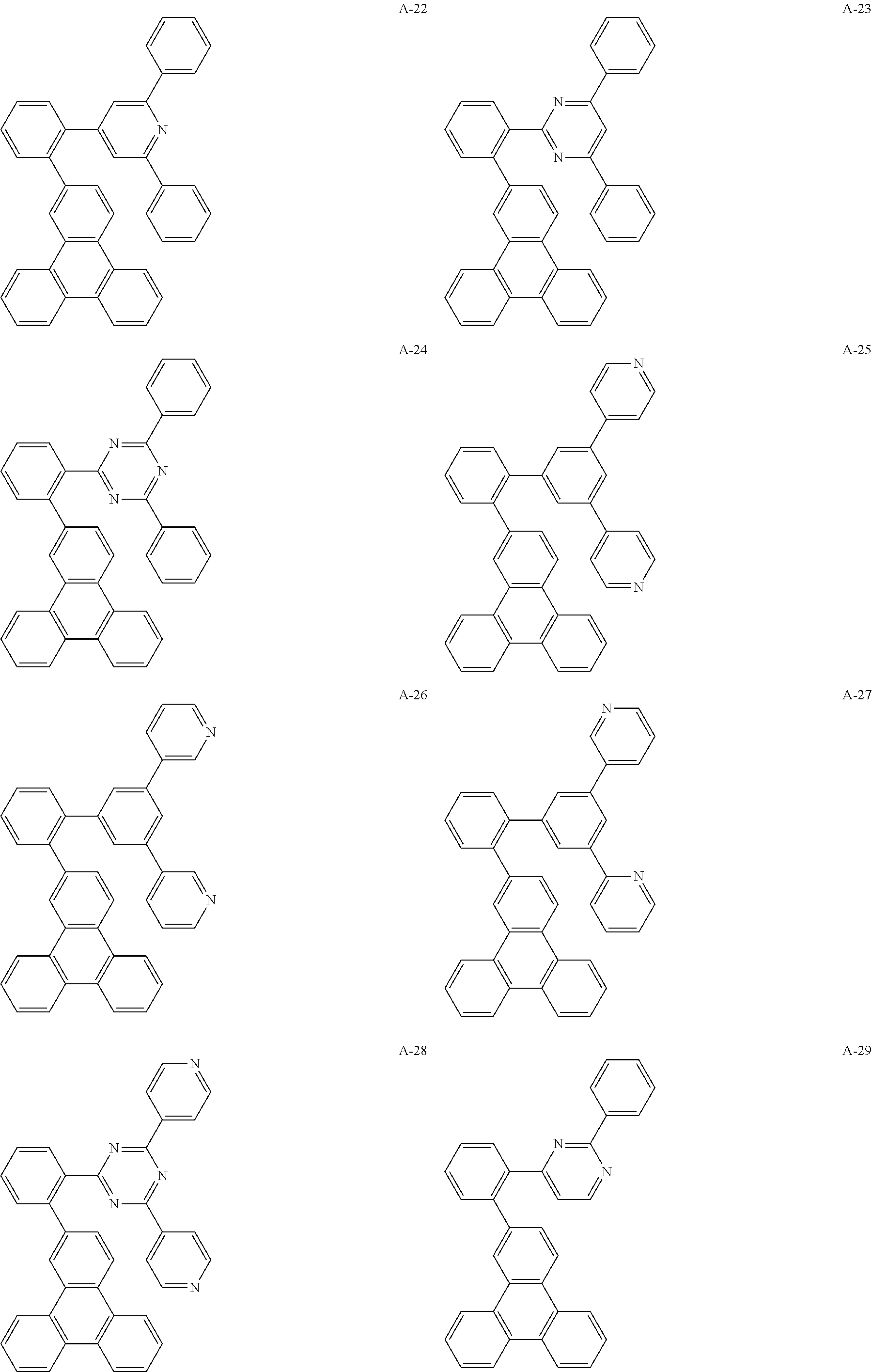

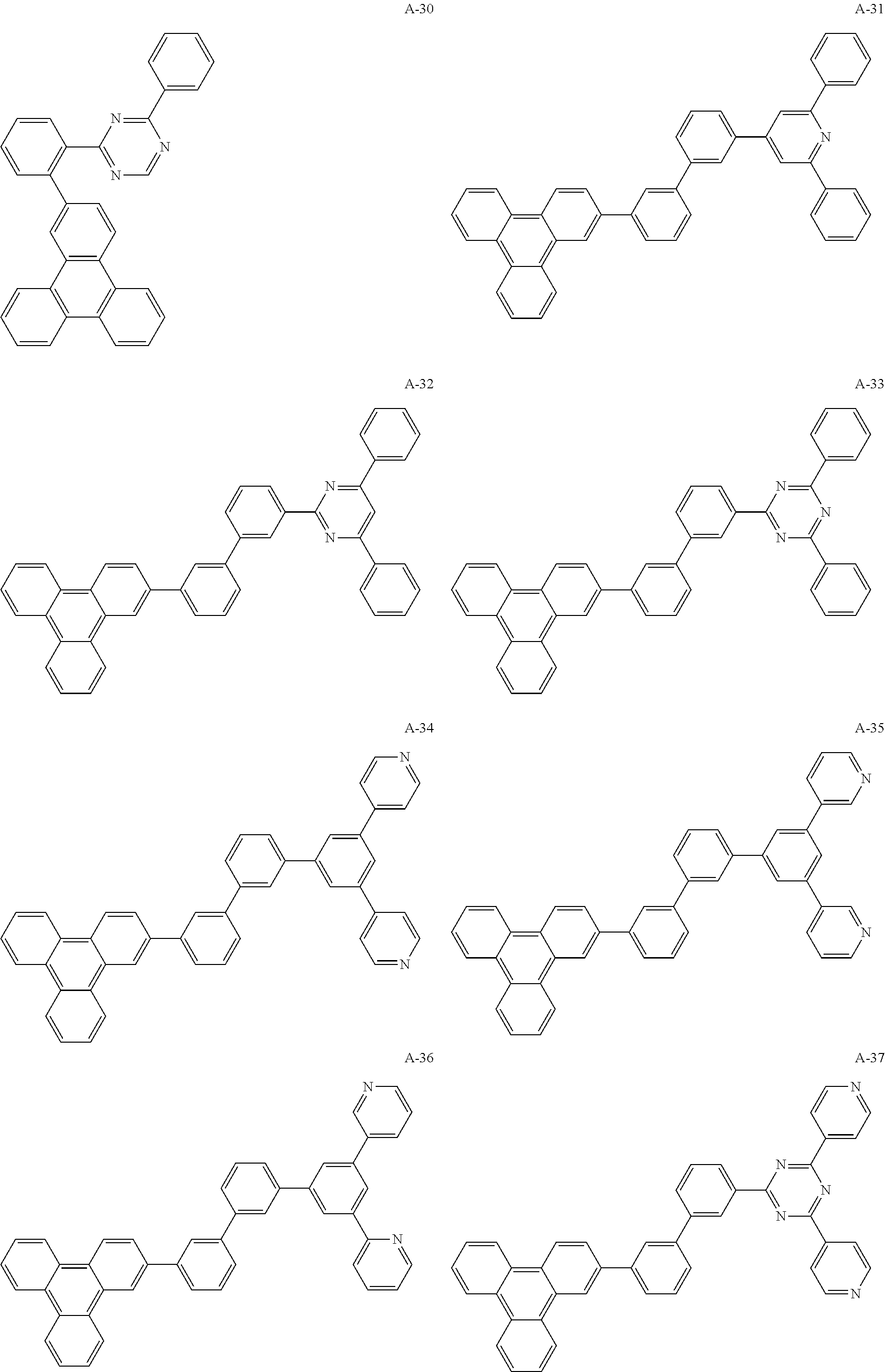

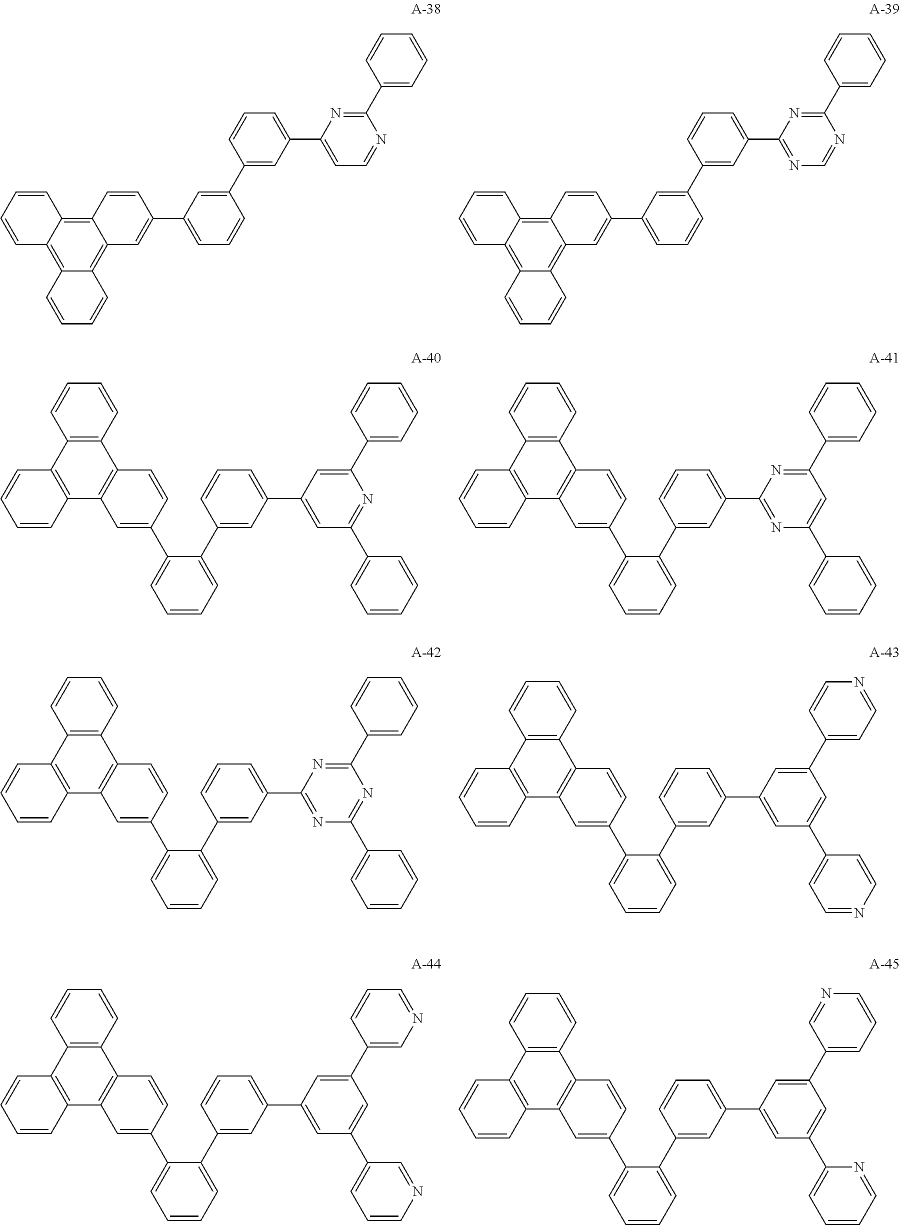

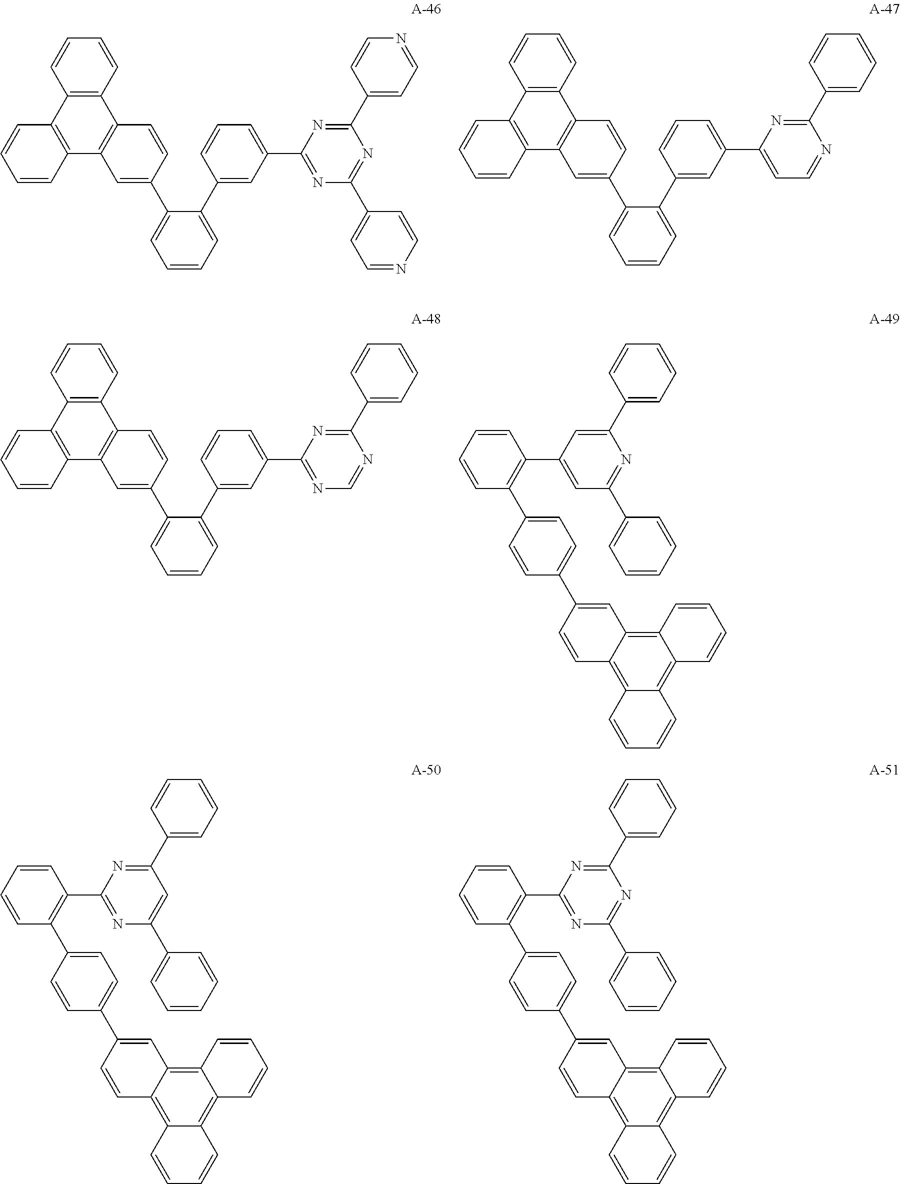

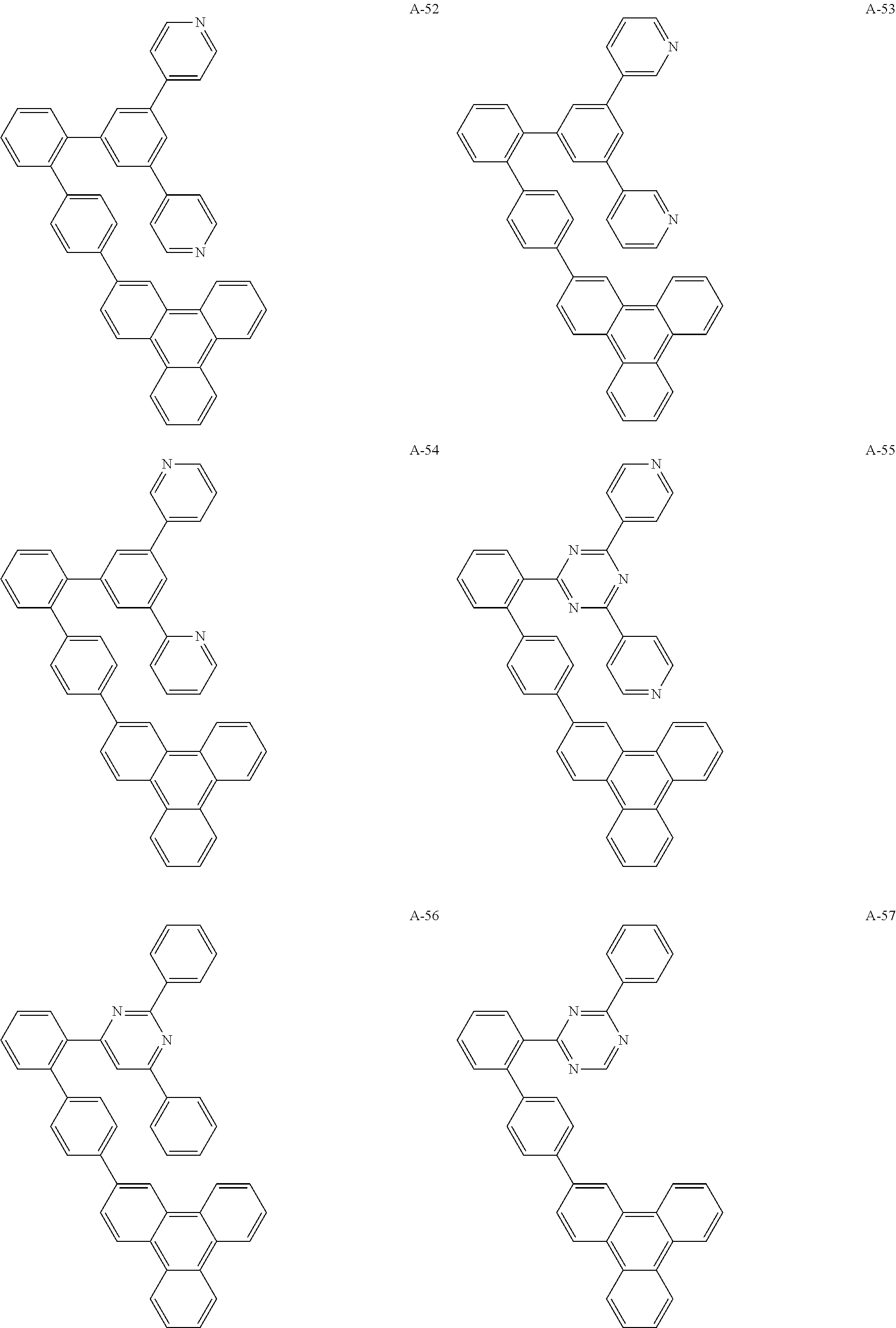

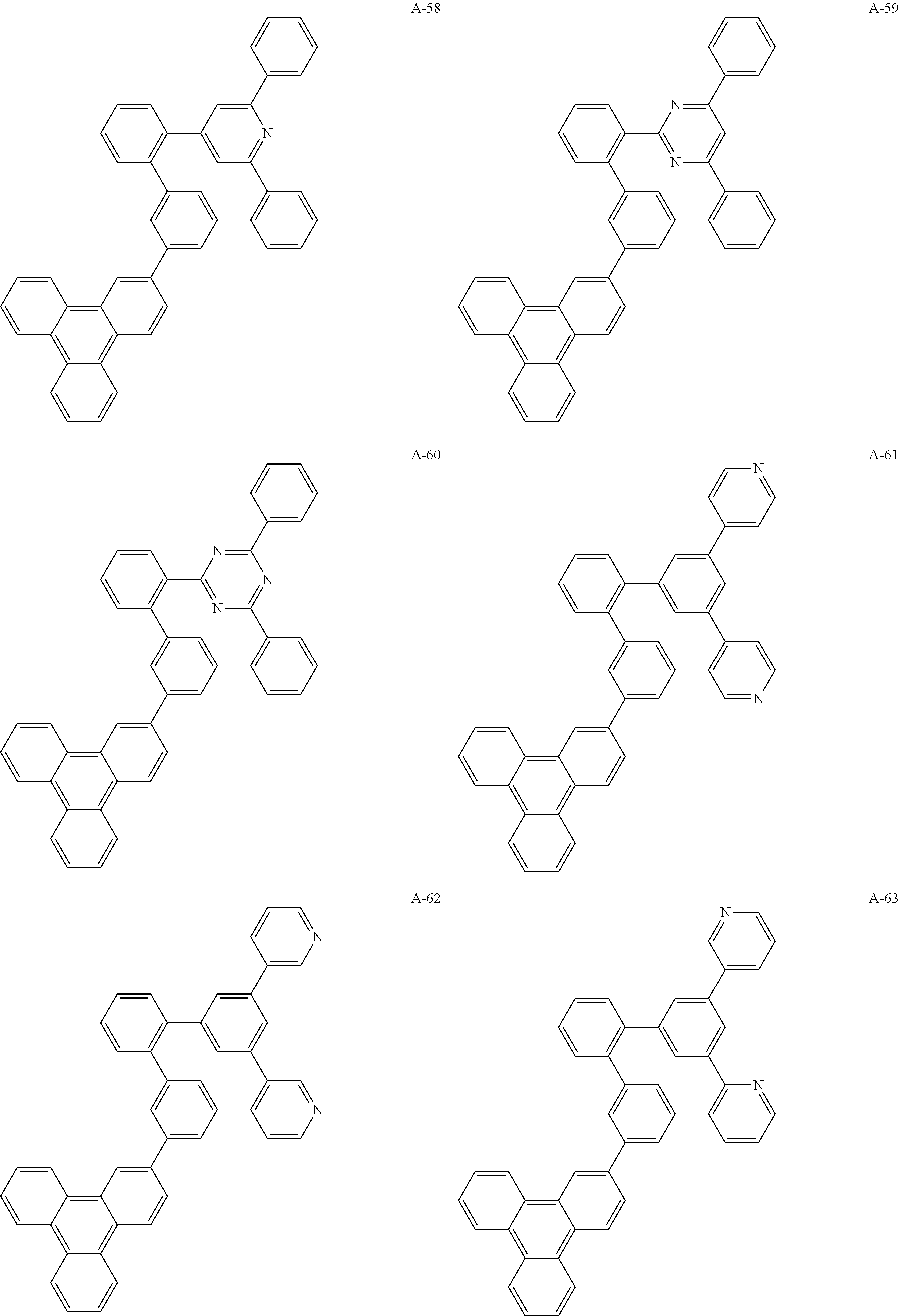 [SMILES: c1ccc(-c2cc(-c3ccccc3-c3cccc(-c4ccc5c6ccccc6c6ccccc6c5c4)c3)cc(-c3ccccc3)n2)cc1 ; c1ccc(-c2cc(-c3ccccc3)nc(-c3ccccc3-c3cccc(-c4ccc5c6ccccc6c6ccccc6c5c4)c3)n2)cc1 ; c1ccc(-c2nc(-c3ccccc3)nc(-c3ccccc3-c3cccc(-c4ccc5c6ccccc6c6ccccc6c5c4)c3)n2)cc1 ; c1cc(-c2ccc3c4ccccc4c4ccccc4c3c2)cc(-c2ccccc2-c2cc(-c3ccncc3)cc(-c3ccncc3)c2)c1 ; c1cncc(-c2cc(-c3cccnc3)cc(-c3ccccc3-c3cccc(-c4ccc5c6ccccc6c6ccccc6c5c4)c3)c2)c1 ; c1ccc(-c2cc(-c3cccnc3)cc(-c3ccccc3-c3cccc(-c4ccc5c6ccccc6c6ccccc6c5c4)c3)c2)nc1]

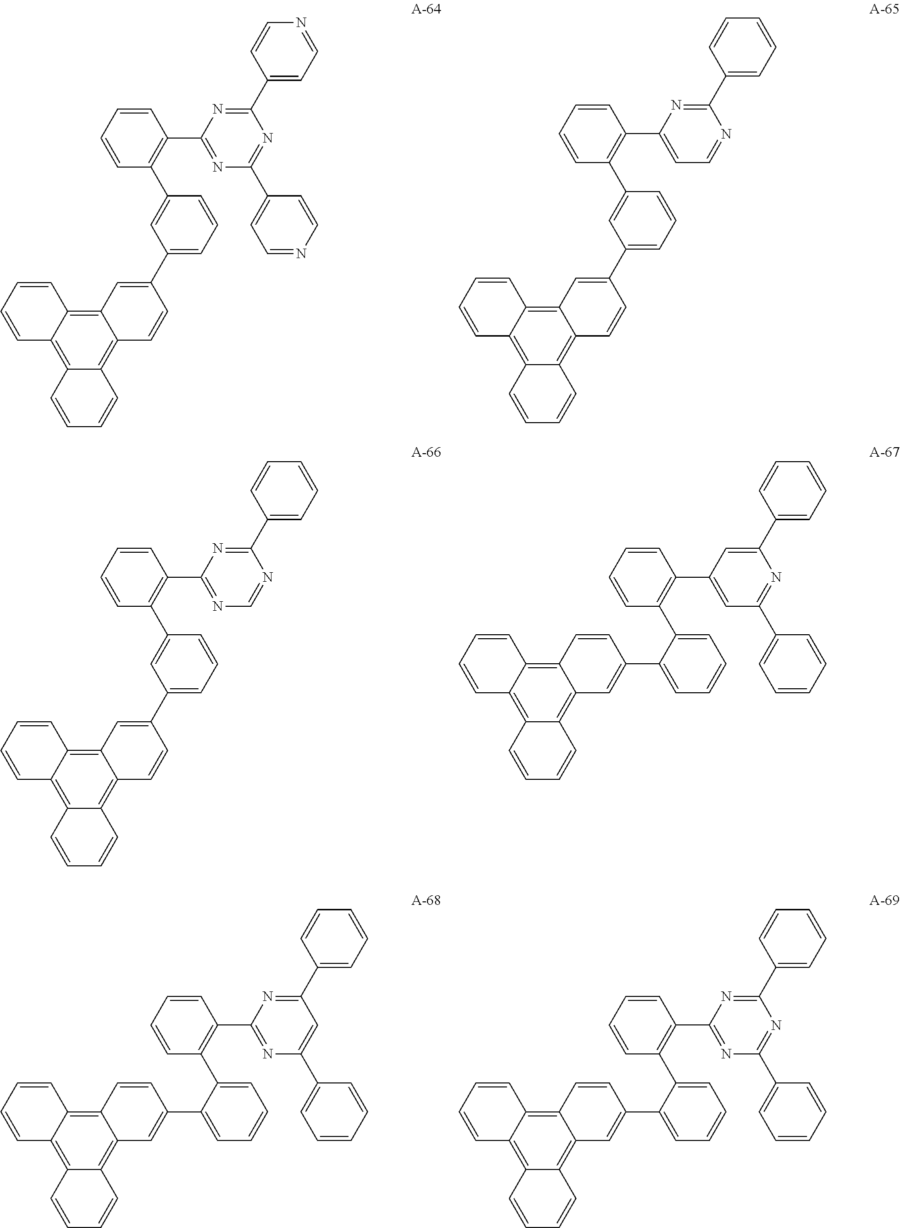 [SMILES: c1cc(-c2ccc3c4ccccc4c4ccccc4c3c2)cc(-c2ccccc2-c2nc(-c3ccncc3)nc(-c3ccncc3)n2)c1 ; c1ccc(-c2nccc(-c3ccccc3-c3cccc(-c4ccc5c6ccccc6c6ccccc6c5c4)c3)n2)cc1 ; c1ccc(-c2ncnc(-c3ccccc3-c3cccc(-c4ccc5c6ccccc6c6ccccc6c5c4)c3)n2)cc1 ; c1ccc(-c2cc(-c3ccccc3-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccccc3-c3ccc4c5ccccc5c5ccccc5c4c3)n2)cc1 ; c1ccc(-c2nc(-c3ccccc3)nc(-c3ccccc3-c3ccccc3-c3ccc4c5ccccc5c5ccccc5c4c3)n2)cc1]

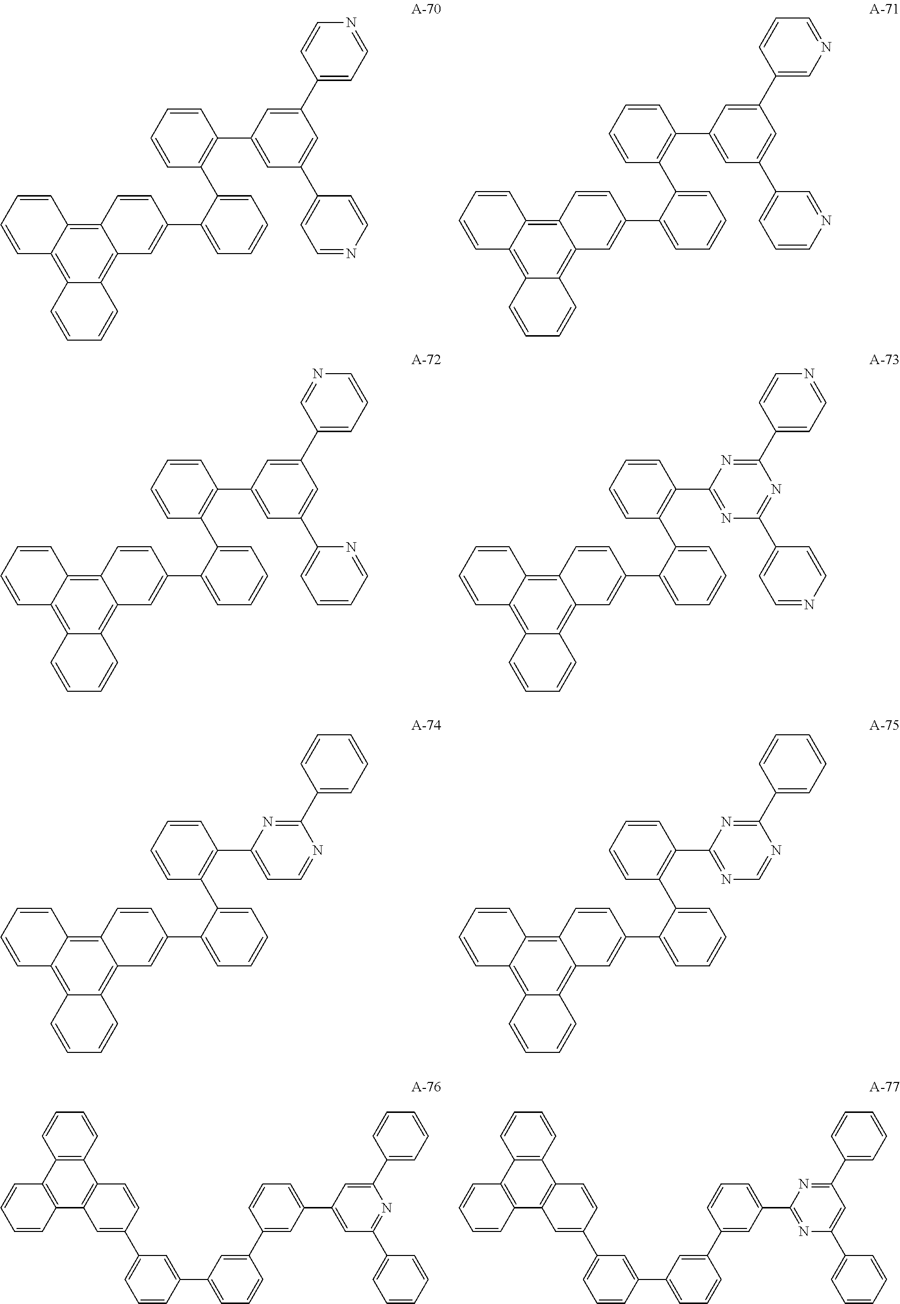

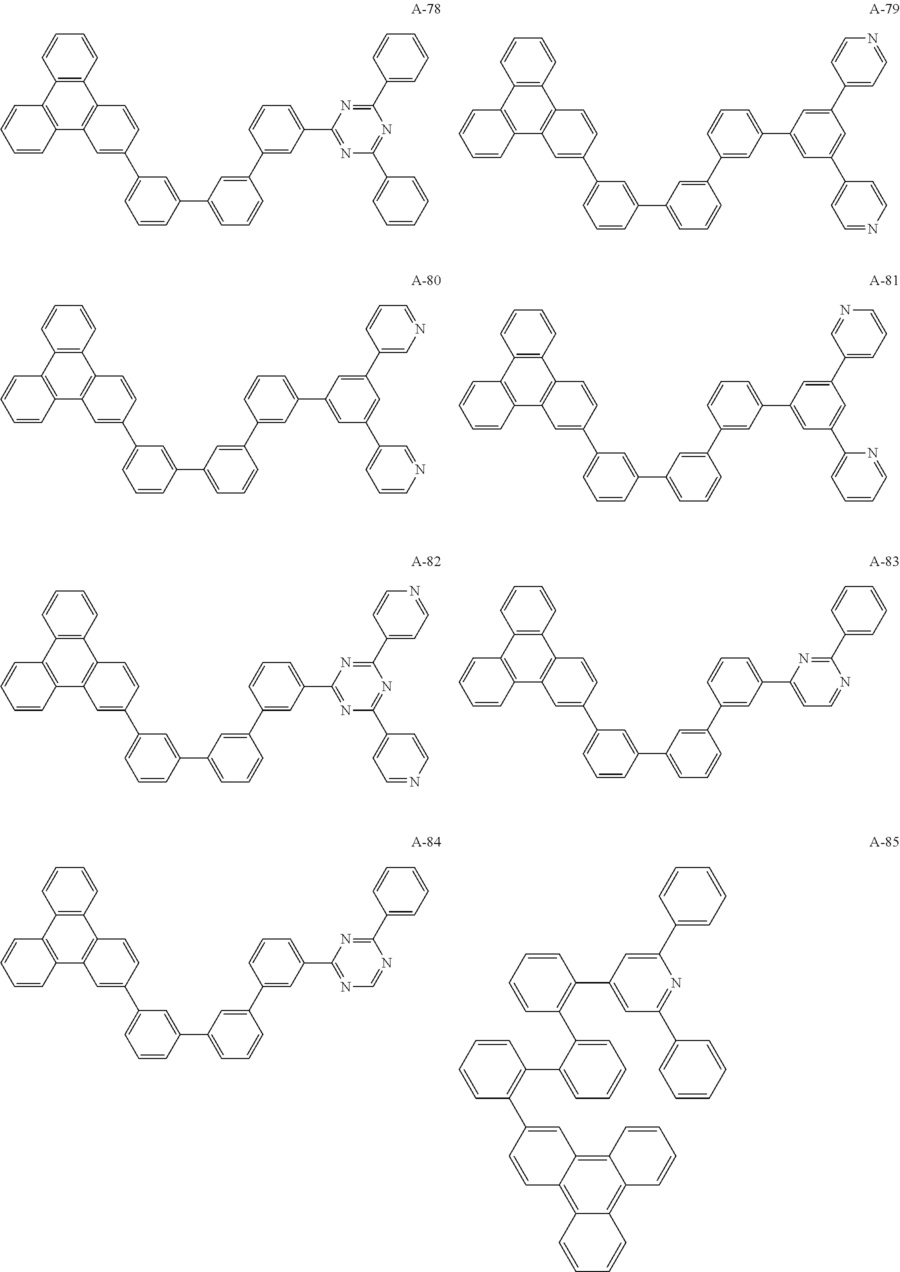

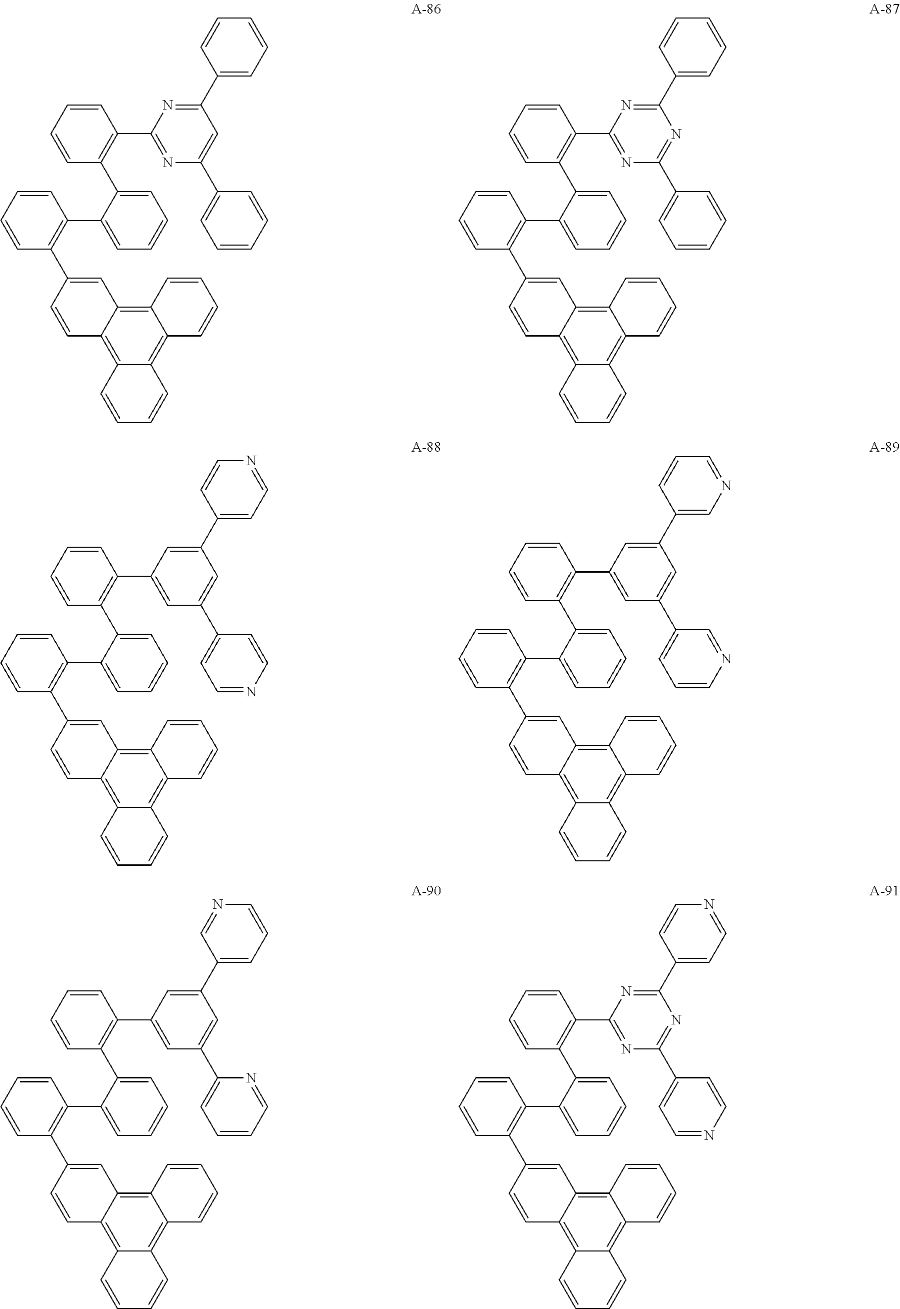

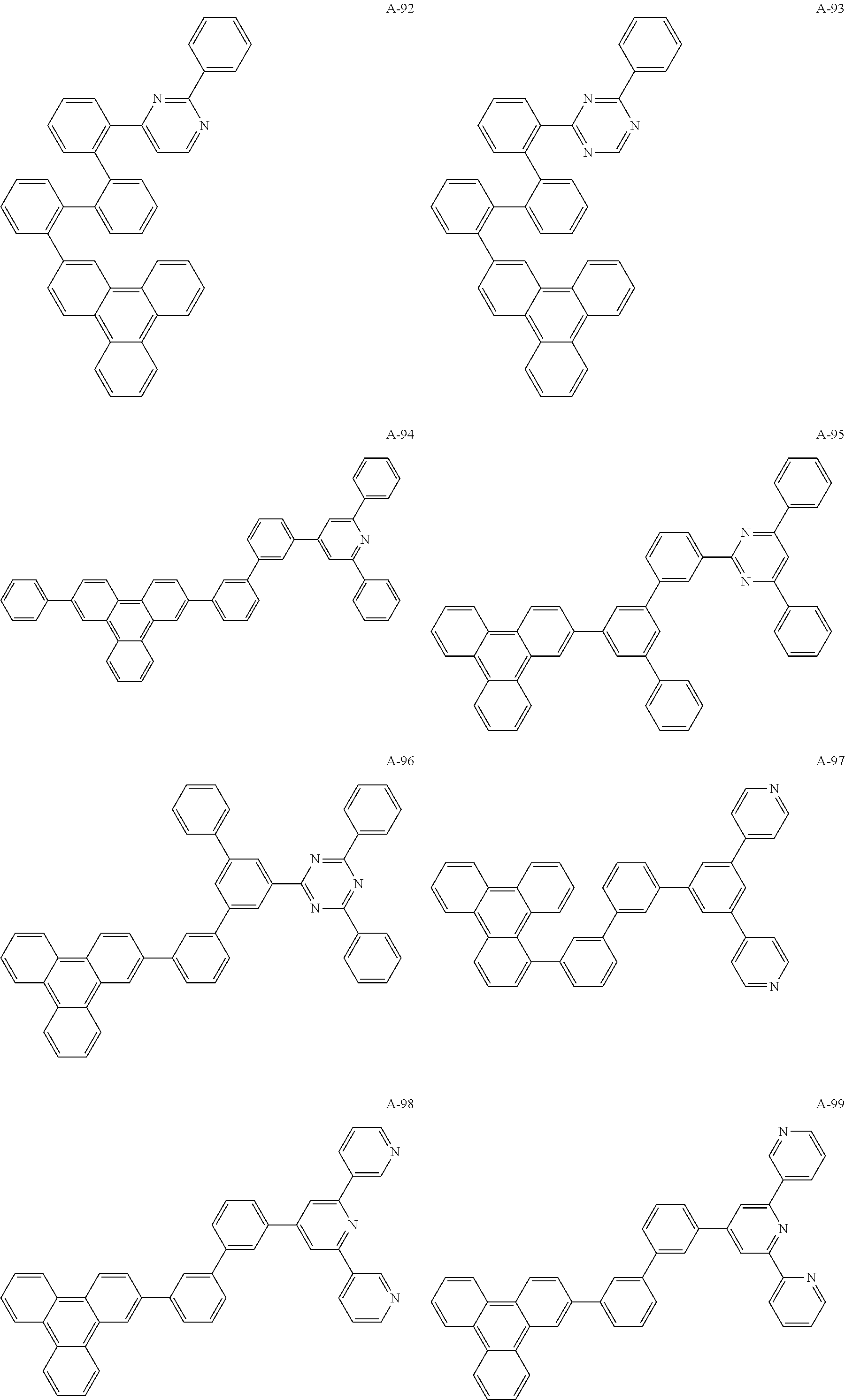

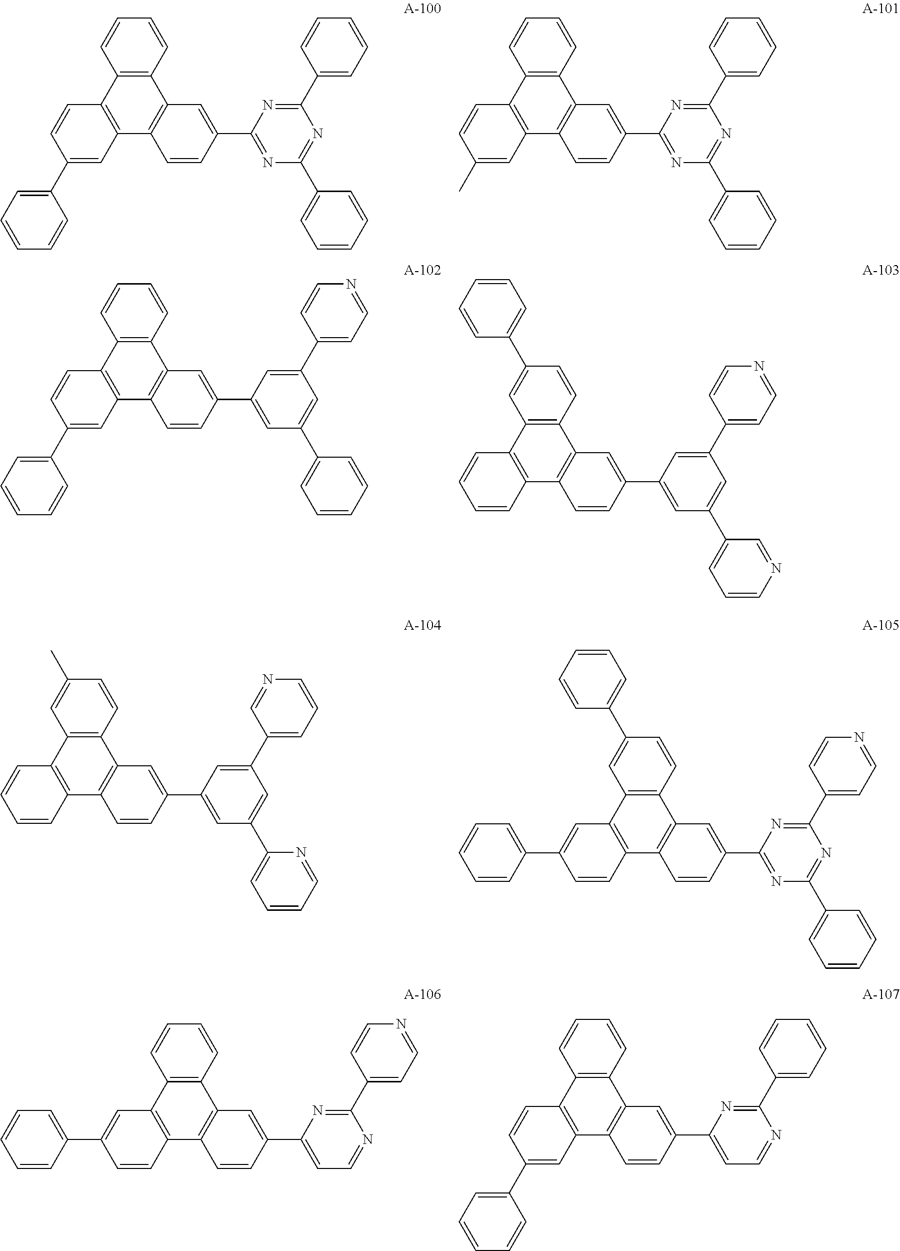

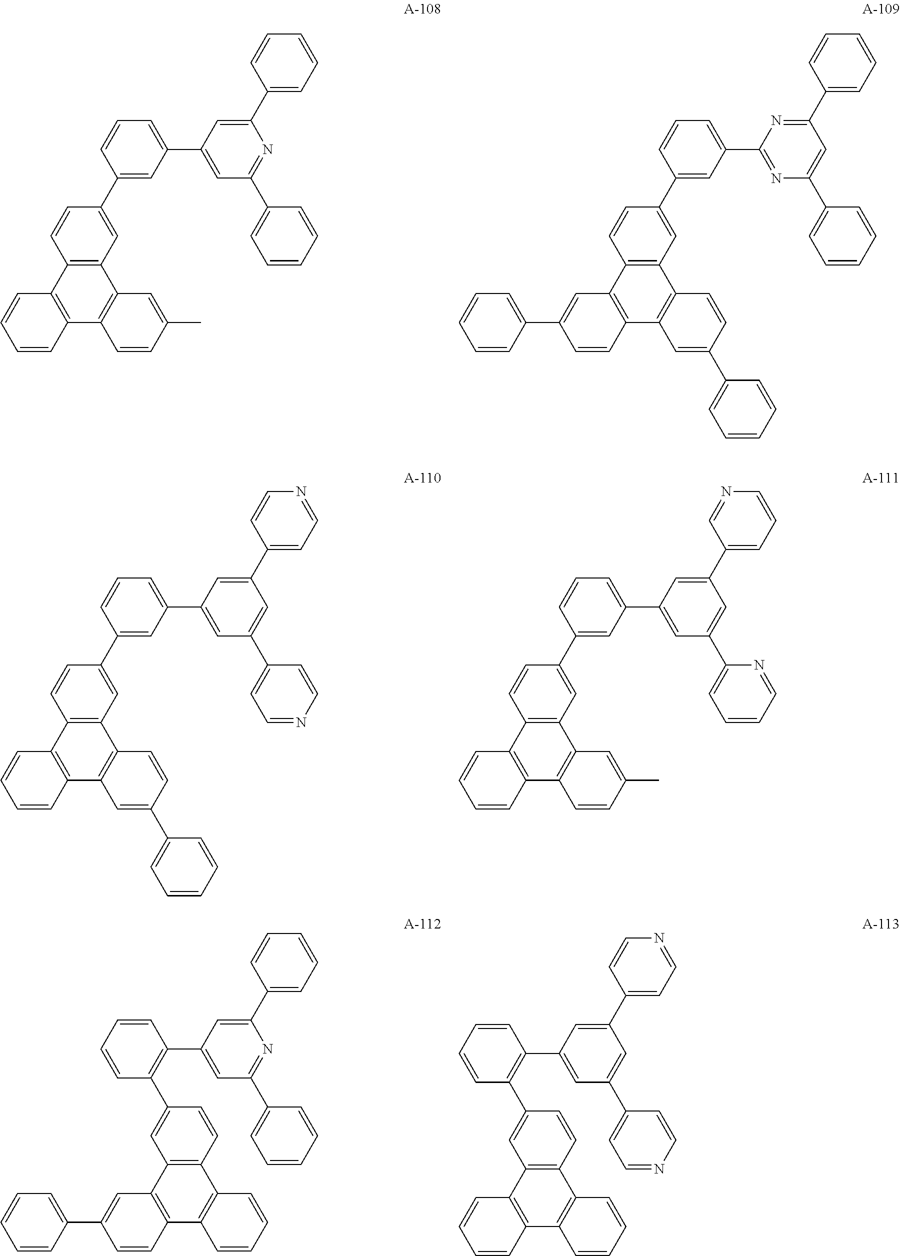 [SMILES: Cc1ccc2c3ccccc3c3ccc(-c4cccc(-c5cc(-c6ccccc6)nc(-c6ccccc6)c5)c4)cc3c2c1 ; c1ccc(-c2ccc3c(c2)c2ccc(-c4cccc(-c5nc(-c6ccccc6)cc(-c6ccccc6)n5)c4)cc2c2ccc(-c4ccccc4)cc32)cc1 ; c1ccc(-c2ccc3c(c2)c2ccccc2c2ccc(-c4cccc(-c5cc(-c6ccncc6)cc(-c6ccncc6)c5)c4)cc23)cc1 ; Cc1ccc2c3ccccc3c3ccc(-c4cccc(-c5cc(-c6cccnc6)cc(-c6ccccn6)c5)c4)cc3c2c1 ; c1ccc(-c2ccc3c4ccccc4c4ccc(-c5ccccc5-c5cc(-c6ccccc6)nc(-c6ccccc6)c5)cc4c3c2)cc1 ; c1ccc(-c2ccc3c4ccccc4c4ccccc4c3c2)c(-c2cc(-c3ccncc3)cc(-c3ccncc3)c2)c1]

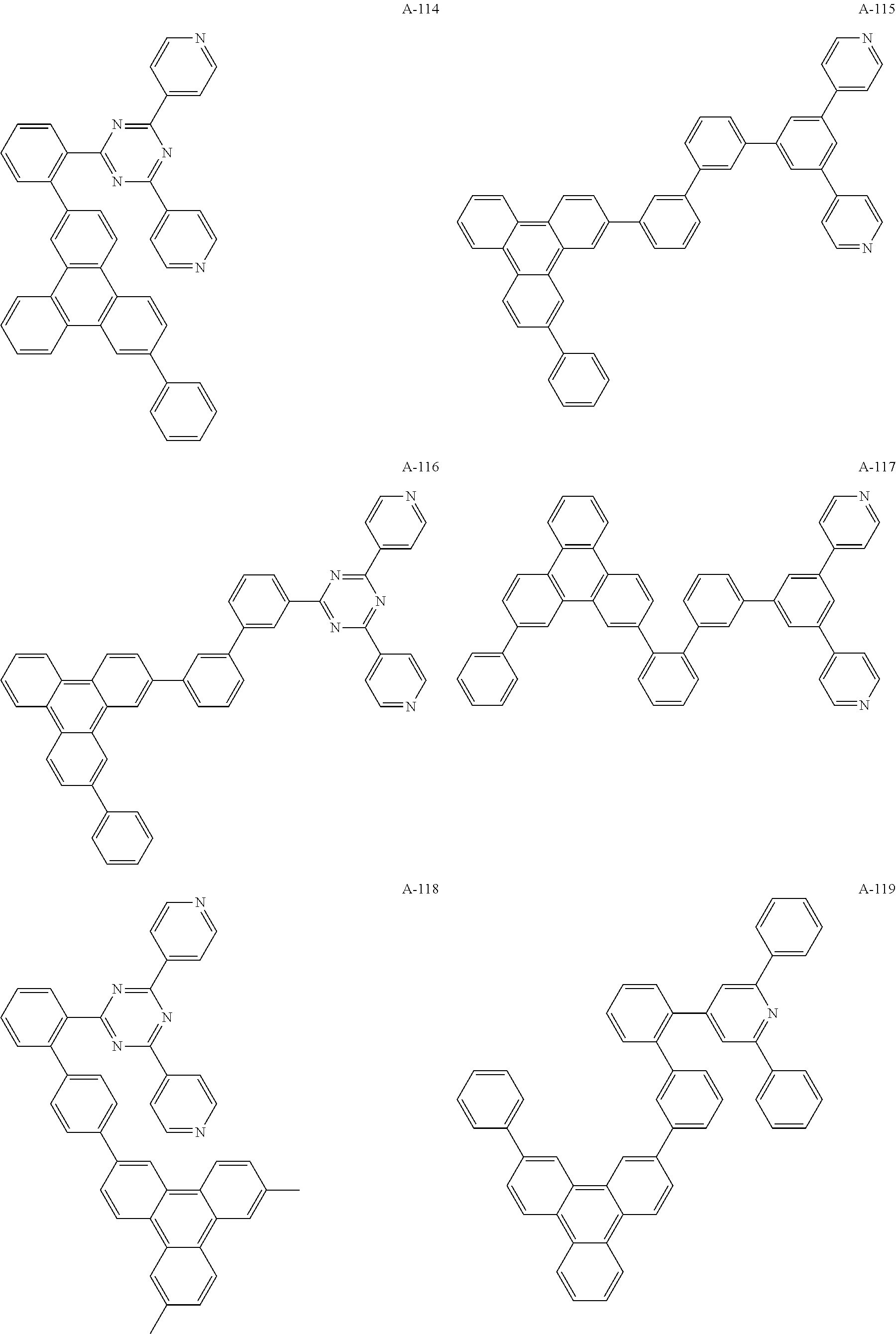

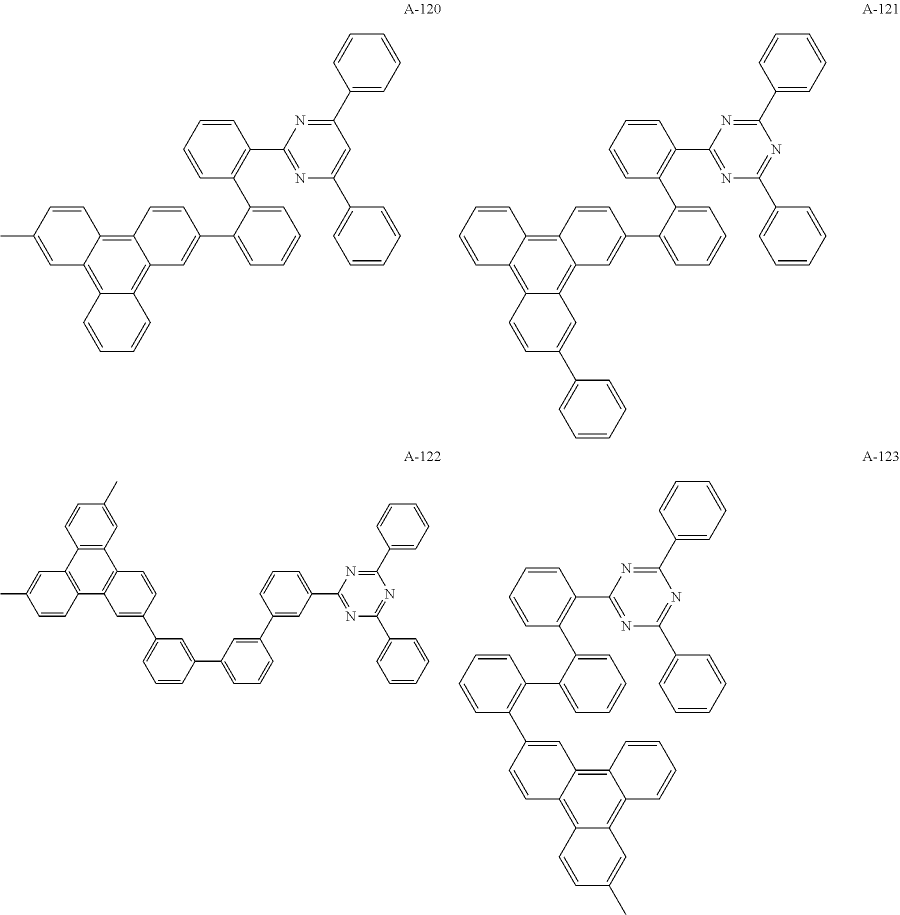

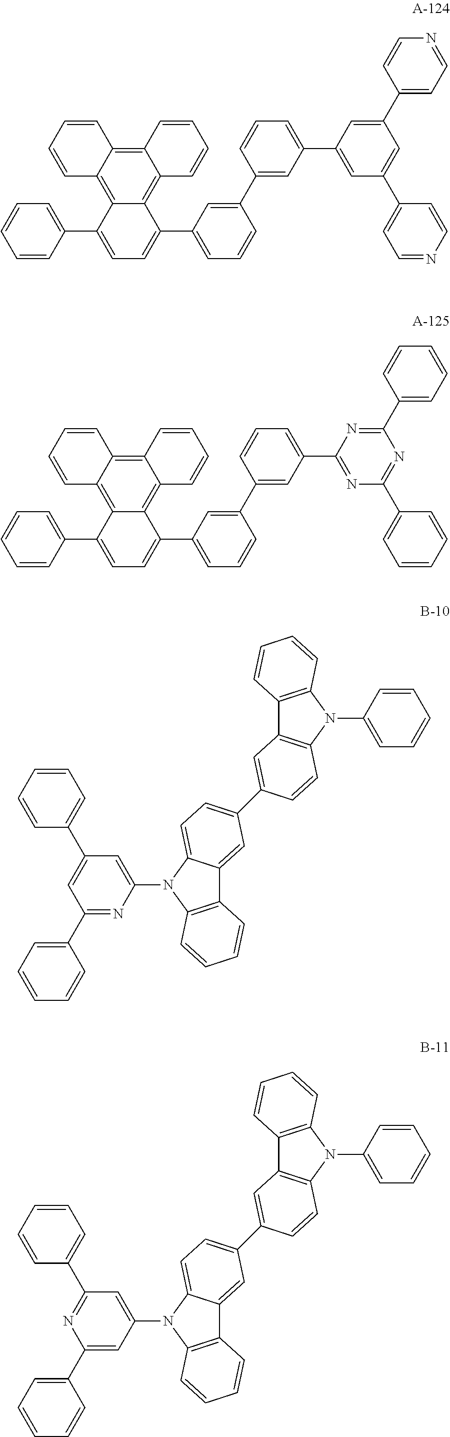 [SMILES: c1ccc(-c2ccc(-c3cccc(-c4cccc(-c5cc(-c6ccncc6)cc(-c6ccncc6)c5)c4)c3)c3c4ccccc4c4ccccc4c23)cc1 ; c1ccc(-c2nc(-c3ccccc3)nc(-c3cccc(-c4cccc(-c5ccc(-c6ccccc6)c6c7ccccc7c7ccccc7c56)c4)c3)n2)cc1 ; c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1]

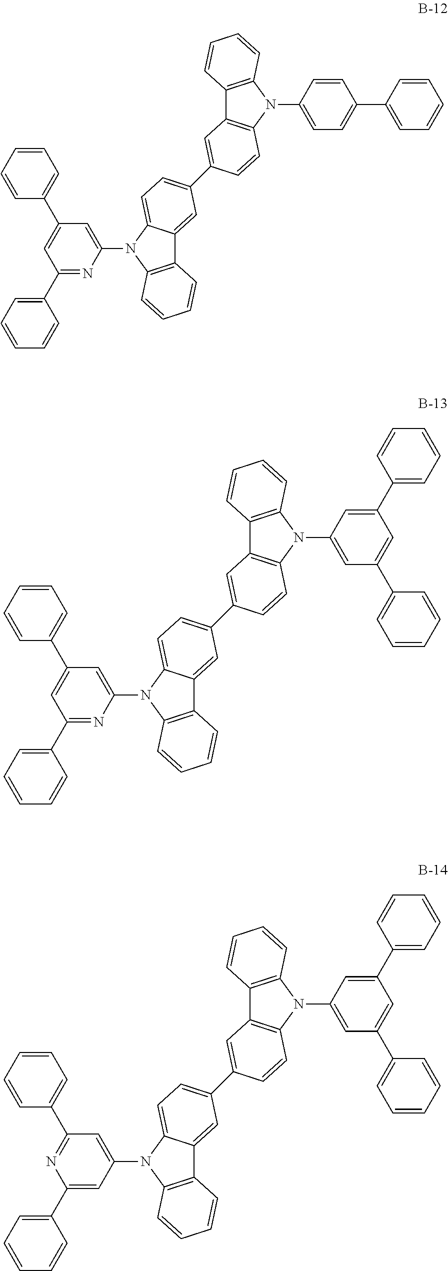 [SMILES: c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1]

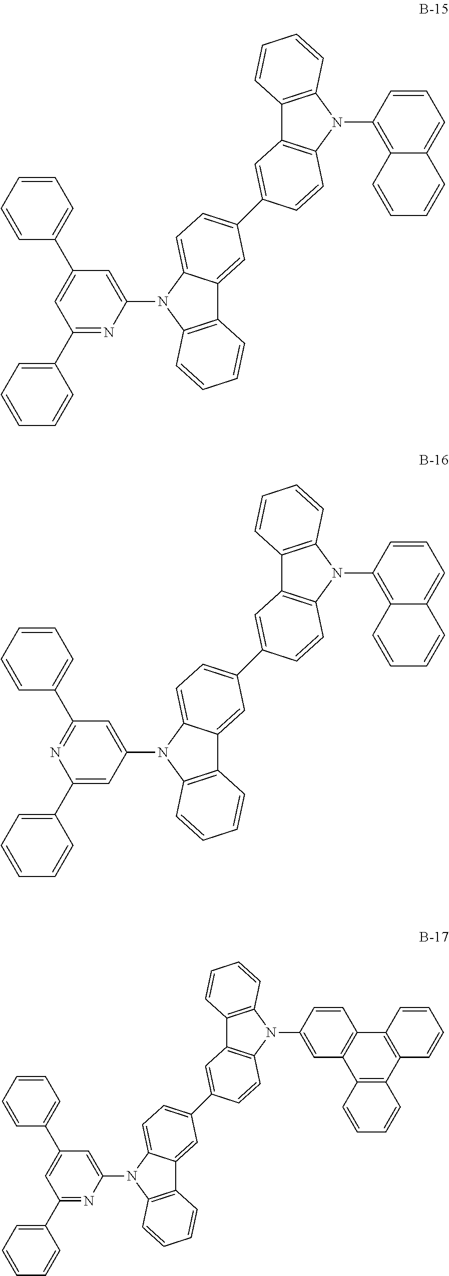 [SMILES: c1ccc(-c2cc(-c3ccccc3)nc(-n3c4ccccc4c4cc(-c5ccc6c(c5)c5ccccc5n6-c5cccc6ccccc56)ccc43)c2)cc1 ; c1ccc(-c2cc(-n3c4ccccc4c4cc(-c5ccc6c(c5)c5ccccc5n6-c5cccc6ccccc56)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1]

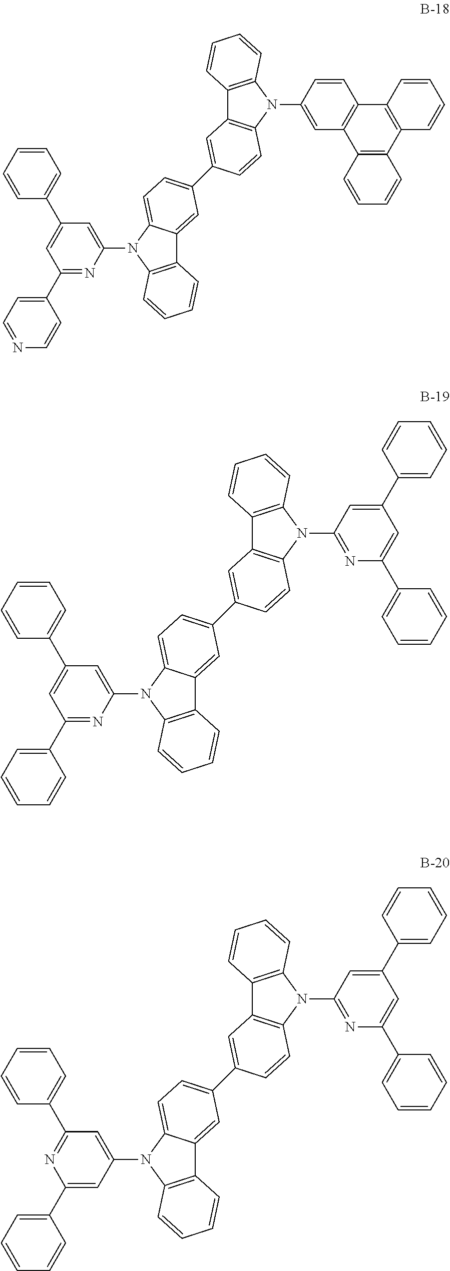 [SMILES: c1ccc(-c2cc(-c3ccncc3)nc(-n3c4ccccc4c4cc(-c5ccc6c(c5)c5ccccc5n6-c5ccc6c7ccccc7c7ccccc7c6c5)ccc43)c2)cc1 ; c1ccc(-c2cc(-c3ccccc3)nc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1]

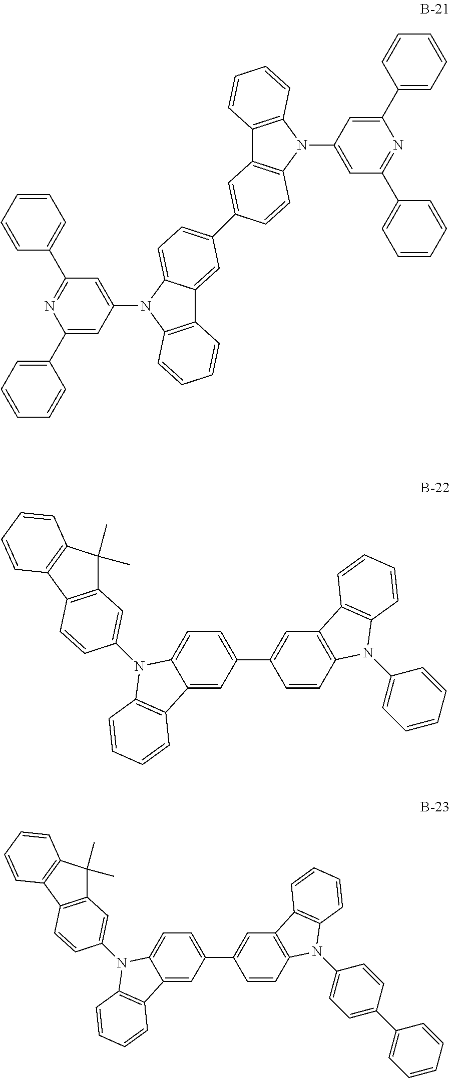 [SMILES: c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1 ; CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21]

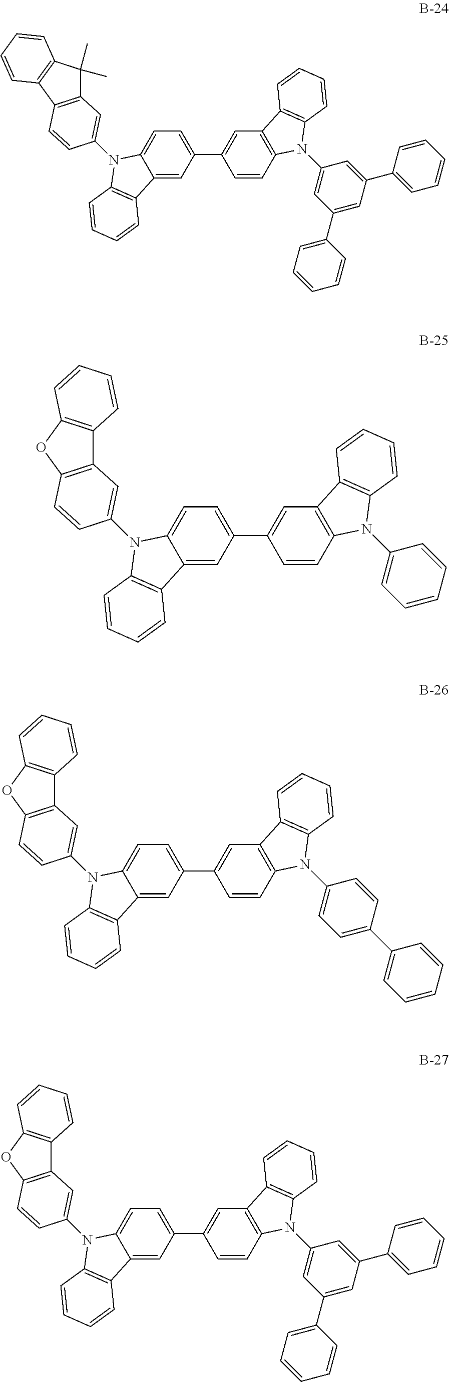 [SMILES: CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21 ; c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1]

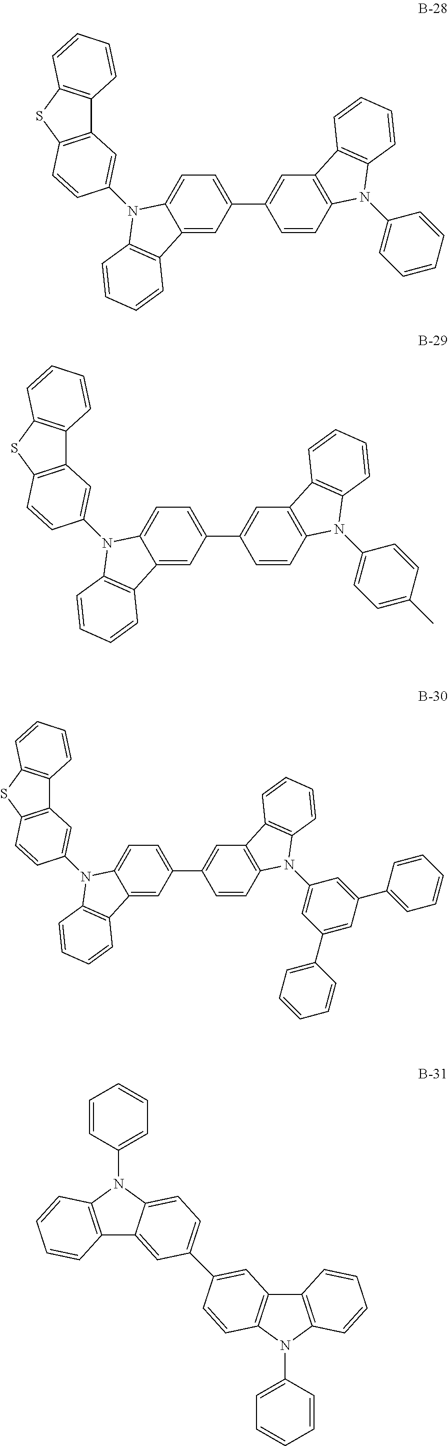 [SMILES: c1ccc(-n2c3ccccc3c3cc(-c4ccc5c(c4)c4ccccc4n5-c4ccc5sc6ccccc6c5c4)ccc32)cc1 ; Cc1ccc(-n2c3ccccc3c3cc(-c4ccc5c(c4)c4ccccc4n5-c4ccc5sc6ccccc6c5c4)ccc32)cc1 ; c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1]

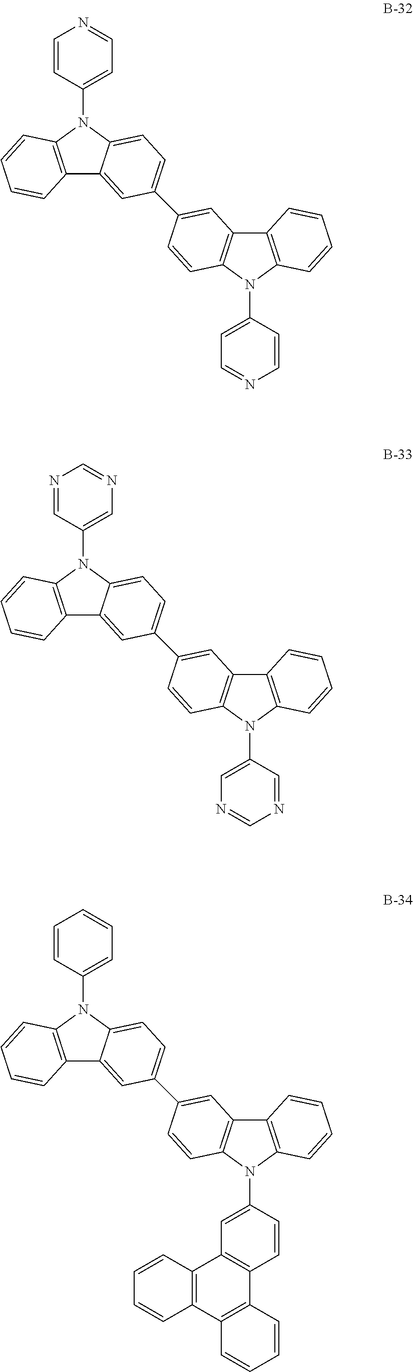 [SMILES: c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1]

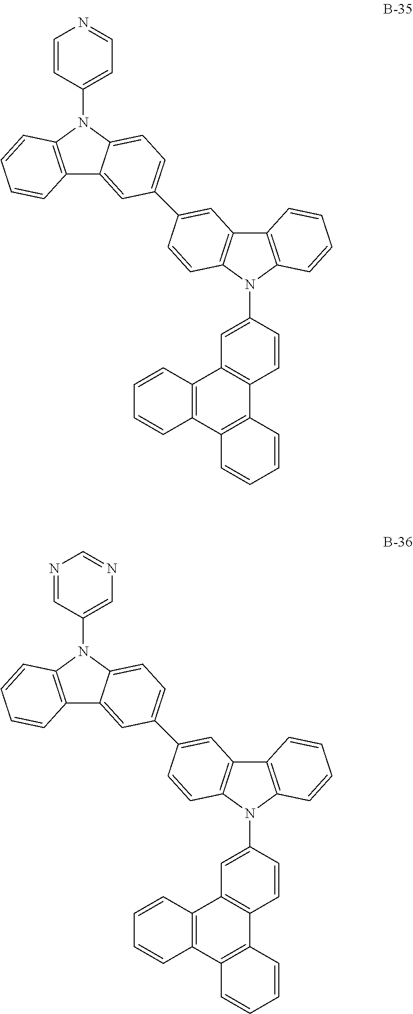 [SMILES: c1ccc2c(c1)c1ccccc1c1cc(-n3c4ccccc4c4cc(-c5ccc6c(c5)c5ccccc5n6-c5ccncc5)ccc43)ccc21 ; c1ccc2c(c1)c1ccccc1c1cc(-n3c4ccccc4c4cc(-c5ccc6c(c5)c5ccccc5n6-c5cncnc5)ccc43)ccc21]

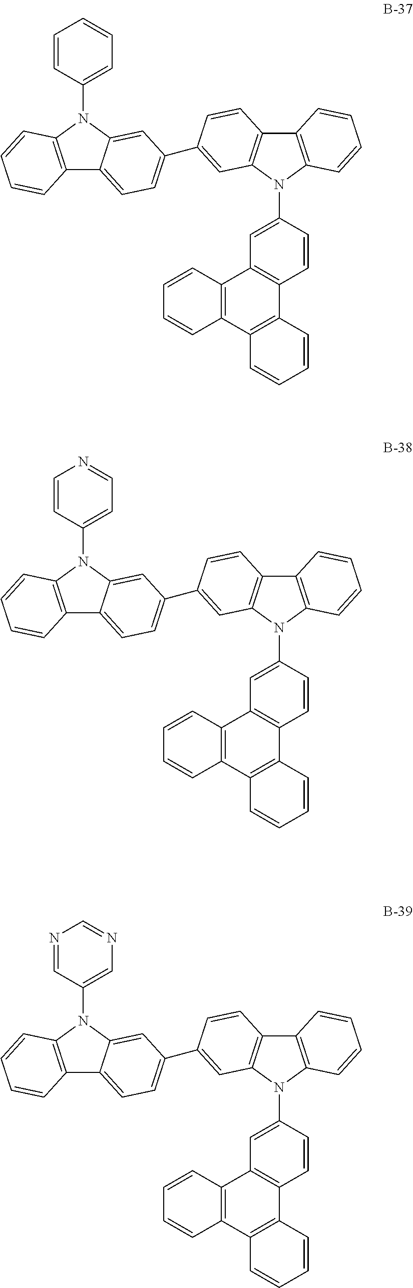 [SMILES: c1ccc(-n2c3ccccc3c3ccc(-c4ccc5c6ccccc6n(-c6ccc7c8ccccc8c8ccccc8c7c6)c5c4)cc32)cc1 ; c1ccc2c(c1)c1ccccc1c1cc(-n3c4ccccc4c4ccc(-c5ccc6c7ccccc7n(-c7ccncc7)c6c5)cc43)ccc21 ; c1ccc2c(c1)c1ccccc1c1cc(-n3c4ccccc4c4ccc(-c5ccc6c7ccccc7n(-c7cncnc7)c6c5)cc43)ccc21]

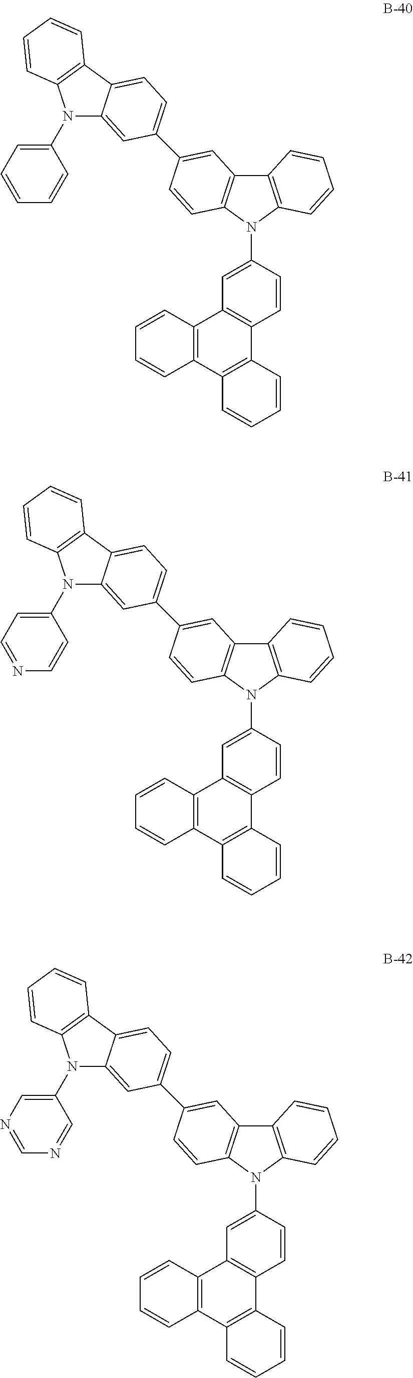 [SMILES: c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)c1ccccc1c1cc(-n3c4ccccc4c4cc(-c5ccc6c7ccccc7n(-c7ccncc7)c6c5)ccc43)ccc21 ; c1ccc2c(c1)c1ccccc1c1cc(-n3c4ccccc4c4cc(-c5ccc6c7ccccc7n(-c7cncnc7)c6c5)ccc43)ccc21]

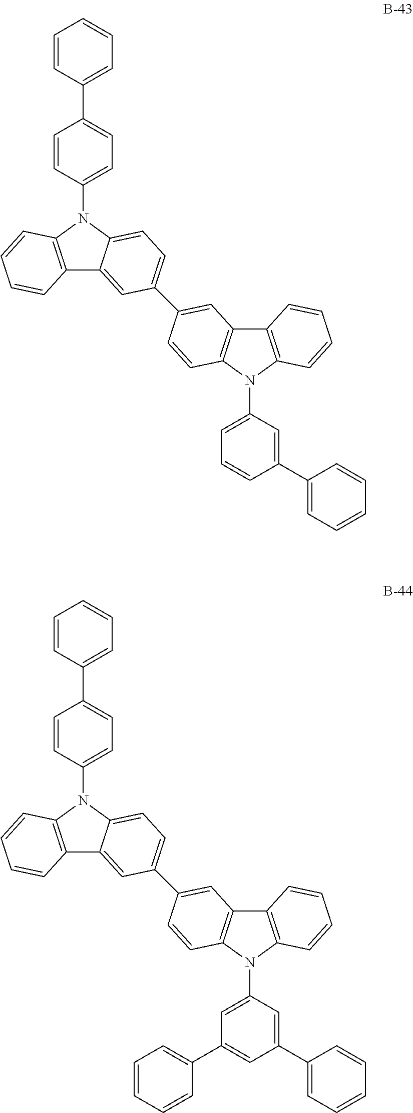 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

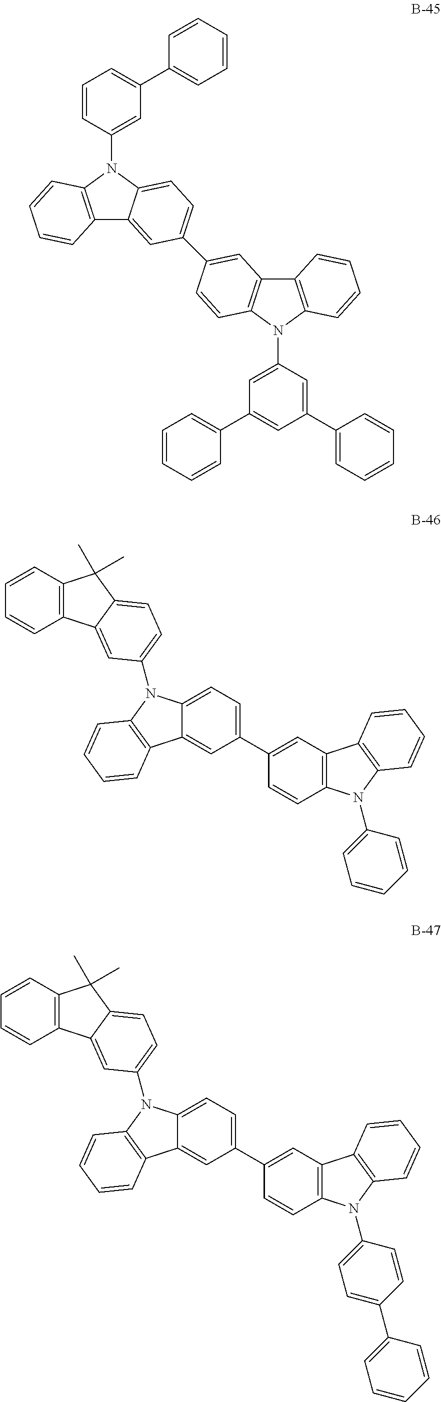 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccc(-c6ccccc6)cc5)ccc43)ccc21]

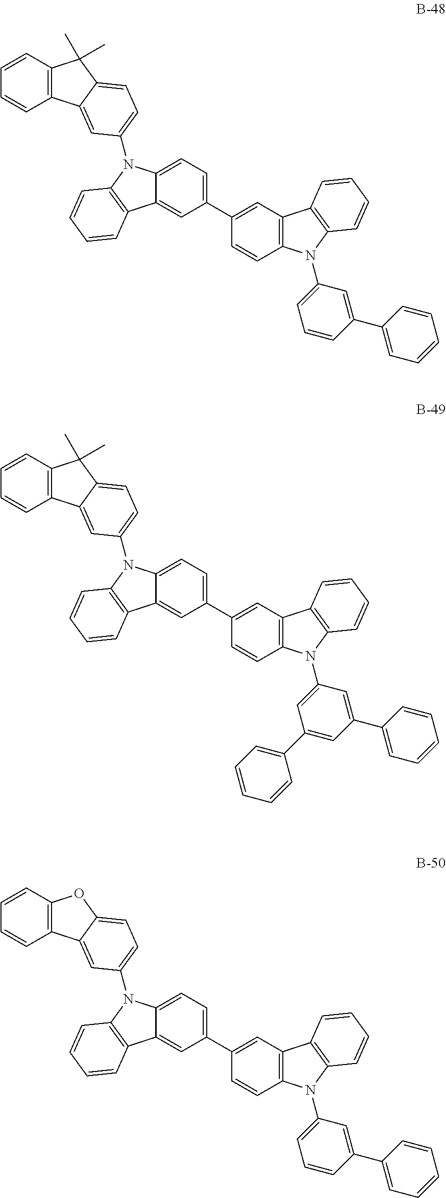 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21 ; c1ccc(-c2cccc(-n3c4ccccc4c4cc(-c5ccc6c(c5)c5ccccc5n6-c5ccc6oc7ccccc7c6c5)ccc43)c2)cc1]

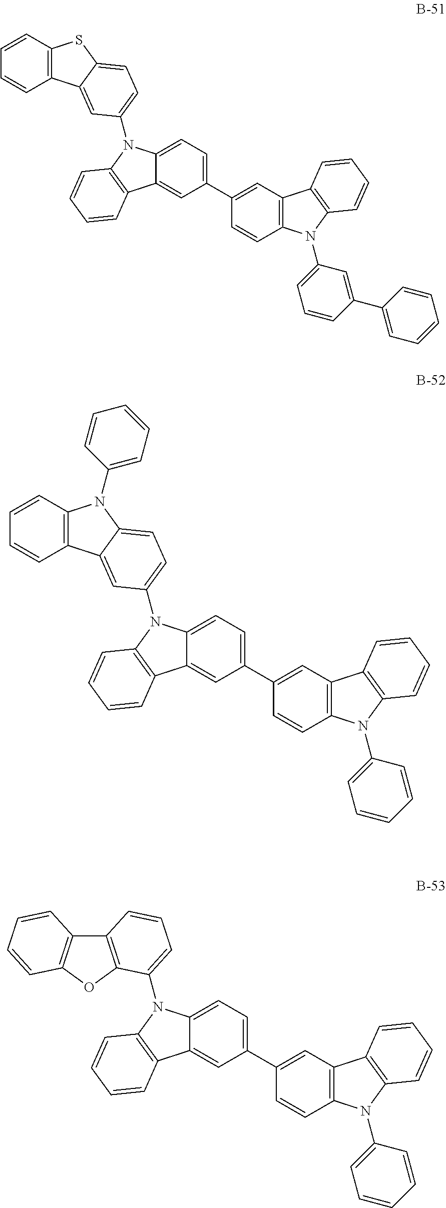 [SMILES: c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5c4oc4ccccc45)ccc32)cc1]

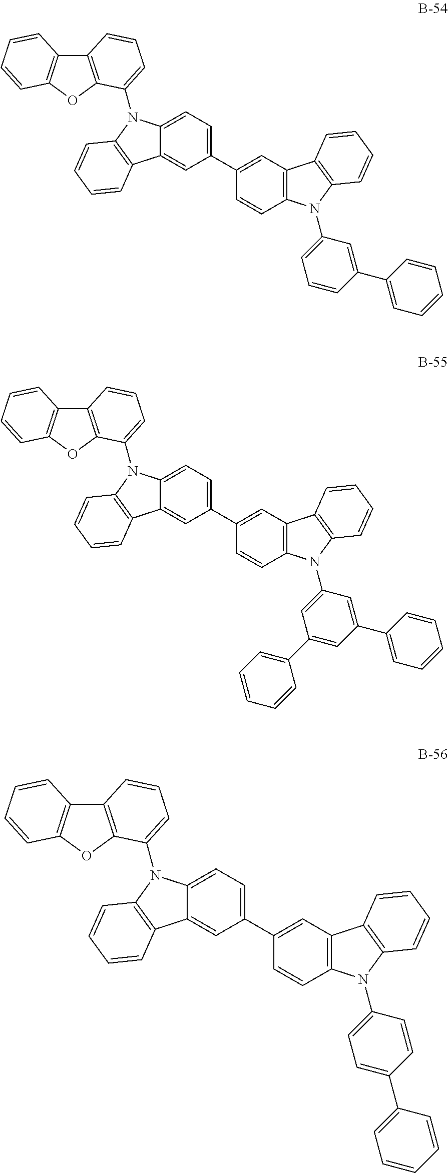 [SMILES: c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5cccc6c5oc5ccccc56)ccc43)cc2)cc1]

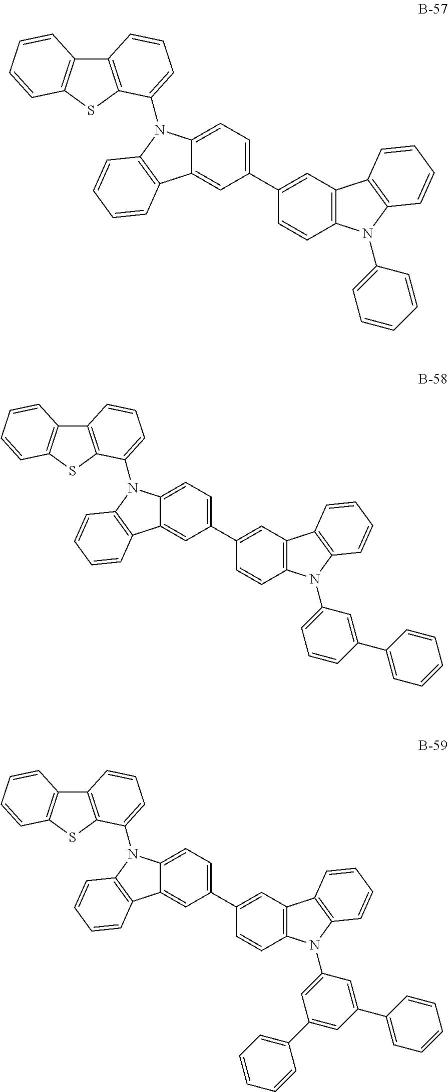 [SMILES: c1ccc(-n2c3ccccc3c3cc(-c4ccc5c(c4)c4ccccc4n5-c4cccc5c4sc4ccccc45)ccc32)cc1 ; c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1]

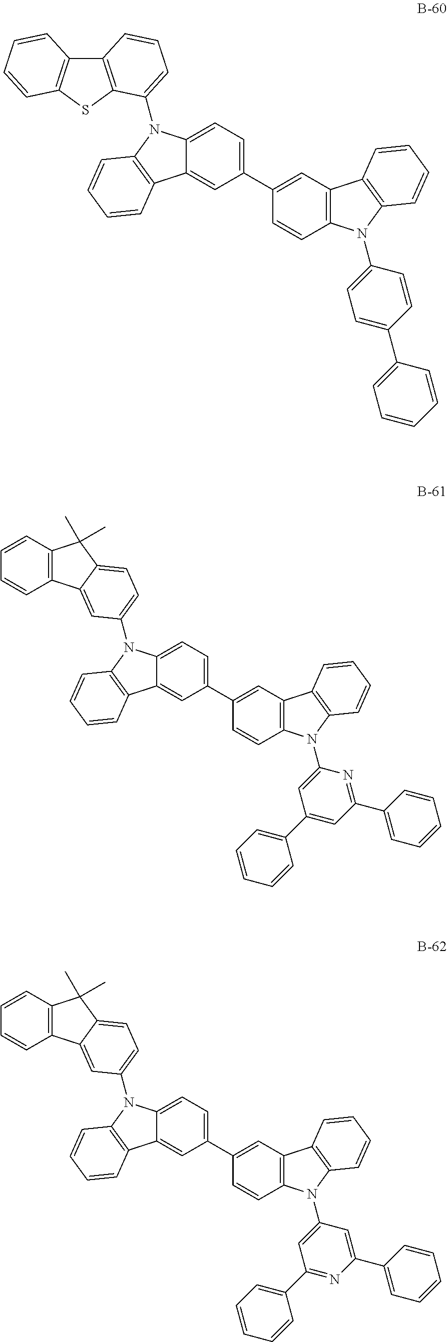 [SMILES: c1ccc(-c2ccc(-n3c4ccccc4c4cc(-c5ccc6c(c5)c5ccccc5n6-c5cccc6c5sc5ccccc56)ccc43)cc2)cc1 ; CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21 ; CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21]

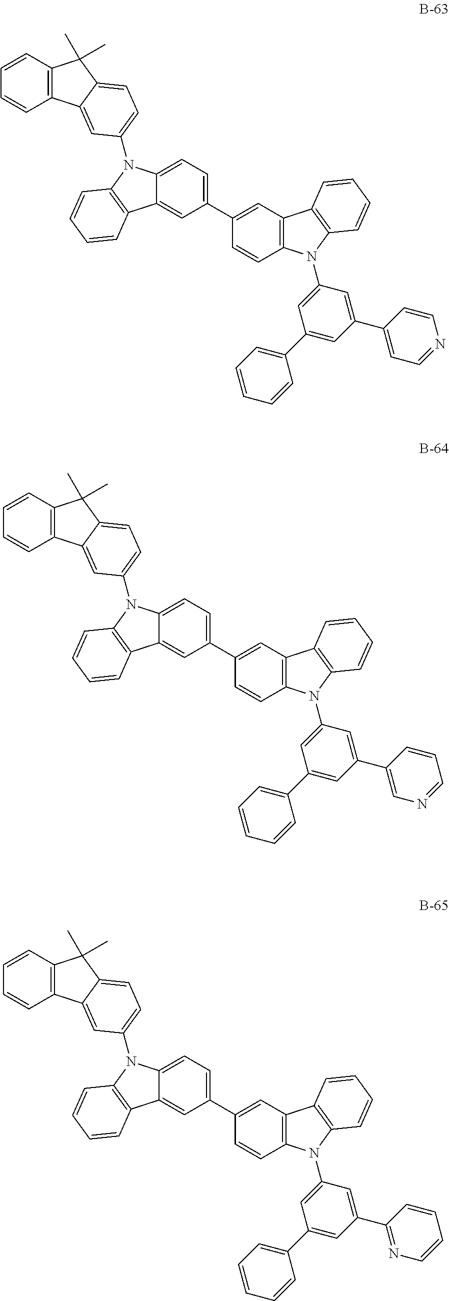 [SMILES: CC1(C)c2ccccc2-c2cc(-n3c4ccccc4c4cc(-c5ccc6c(c5)c5ccccc5n6-c5cc(-c6ccccc6)cc(-c6ccncc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6cccnc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccn6)c5)ccc43)ccc21]

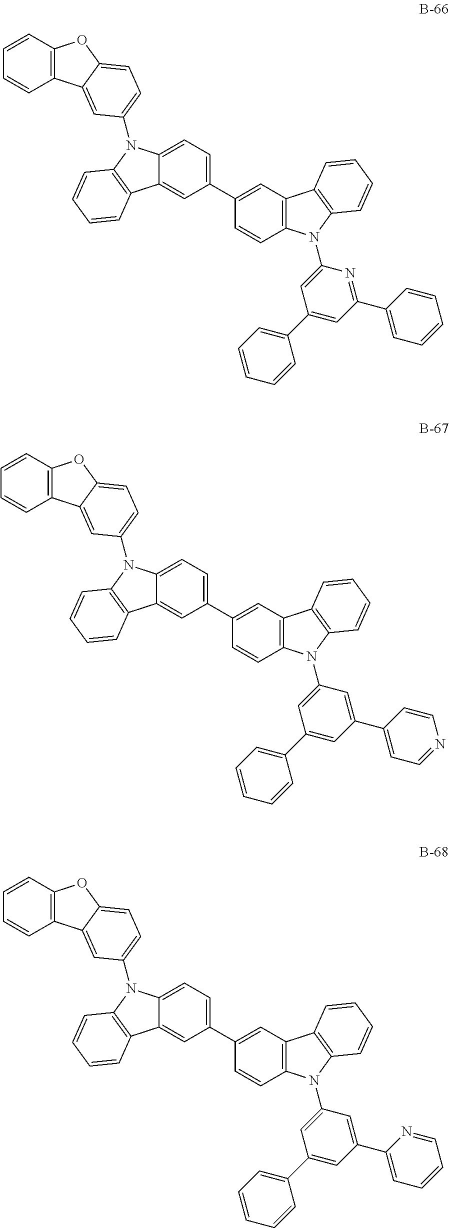 [SMILES: c1ccc(-c2cc(-c3ccccc3)nc(-n3c4ccccc4c4cc(-c5ccc6c(c5)c5ccccc5n6-c5ccc6oc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1]

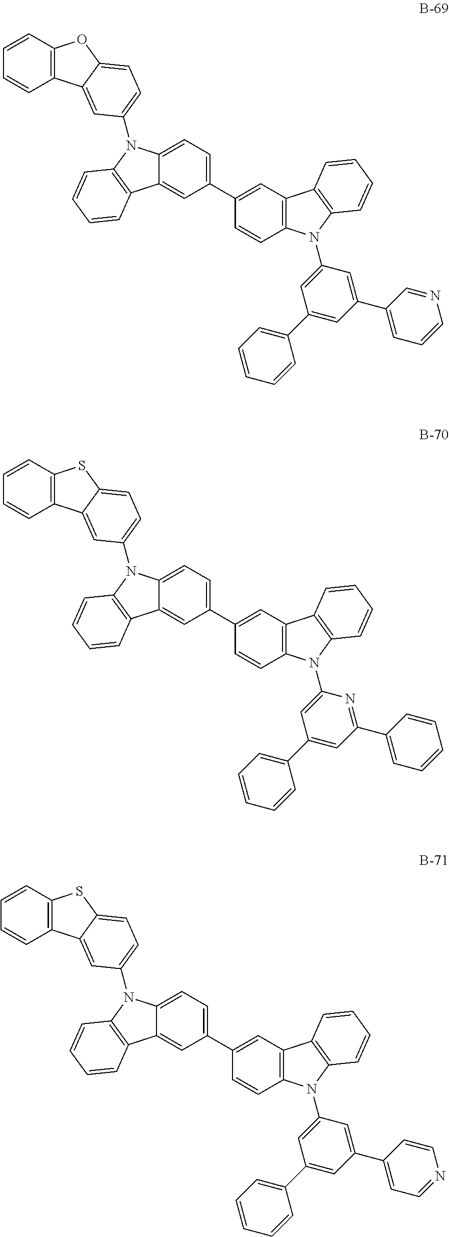 [SMILES: c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1]

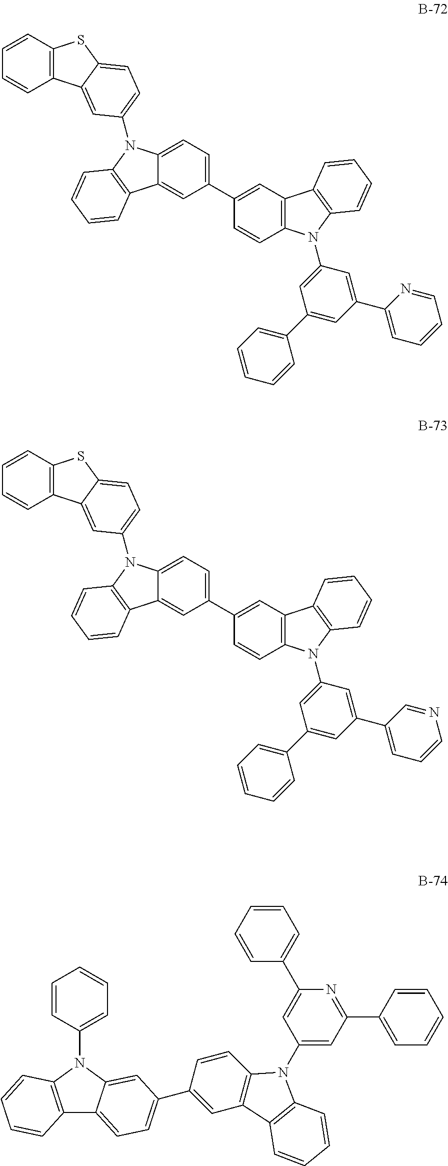 [SMILES: c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1 ; c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1]

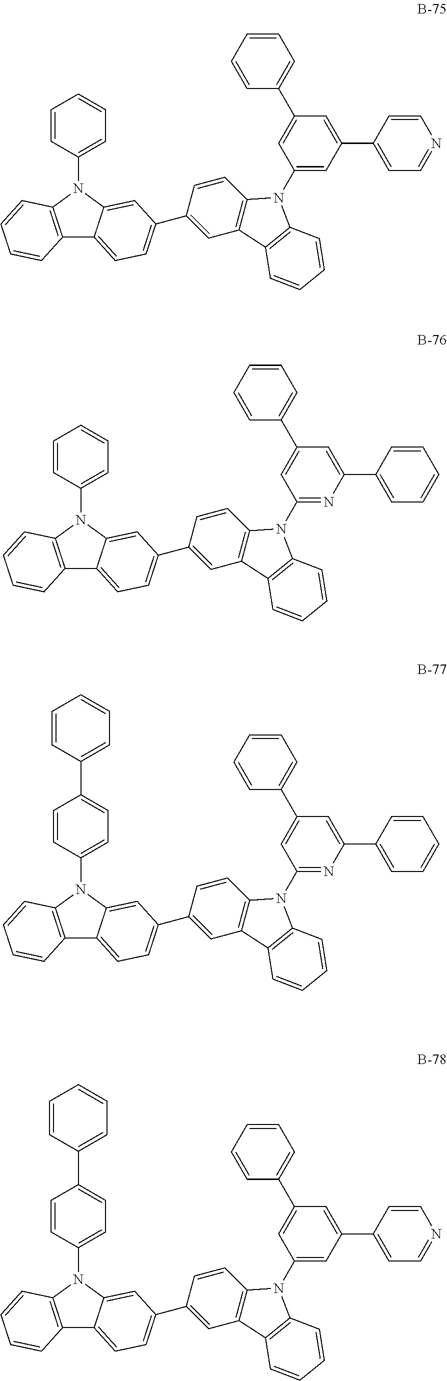 [SMILES: c1ccc(-c2cc(-c3ccncc3)cc(-n3c4ccccc4c4cc(-c5ccc6c7ccccc7n(-c7ccccc7)c6c5)ccc43)c2)cc1 ; c1ccc(-c2cc(-c3ccccc3)nc(-n3c4ccccc4c4cc(-c5ccc6c7ccccc7n(-c7ccccc7)c6c5)ccc43)c2)cc1 ; c1ccc(-c2ccc(-n3c4ccccc4c4ccc(-c5ccc6c(c5)c5ccccc5n6-c5cc(-c6ccccc6)cc(-c6ccccc6)n5)cc43)cc2)cc1 ; c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1]

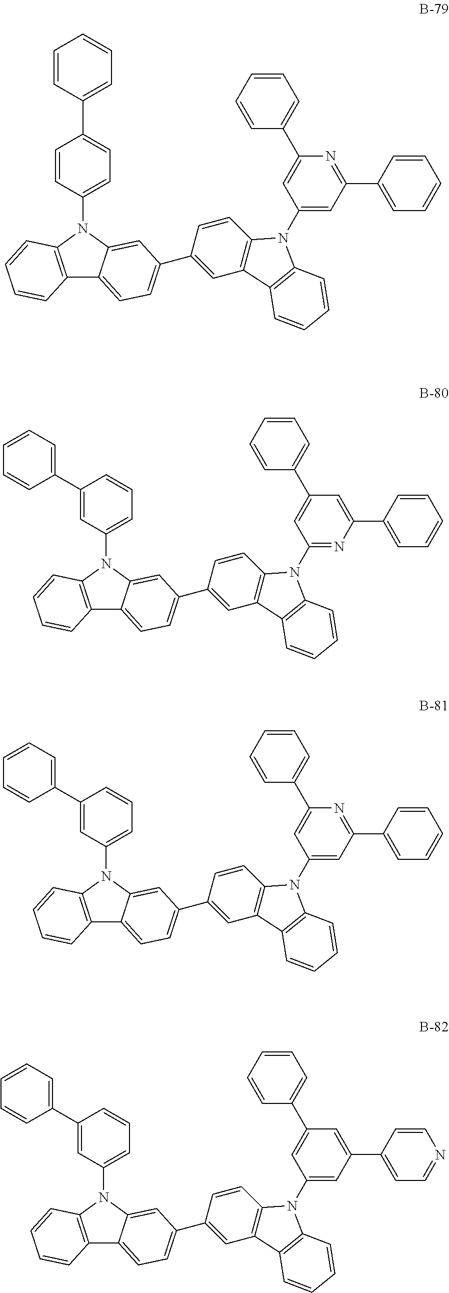 [SMILES: c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1 ; c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1 ; c1ccc(-c2cccc(-n3c4ccccc4c4ccc(-c5ccc6c(c5)c5ccccc5n6-c5cc(-c6ccccc6)nc(-c6ccccc6)c5)cc43)c2)cc1 ; c1ccc(-c2cccc(-n3c4ccccc4c4ccc(-c5ccc6c(c5)c5ccccc5n6-c5cc(-c6ccccc6)cc(-c6ccncc6)c5)cc43)c2)cc1]

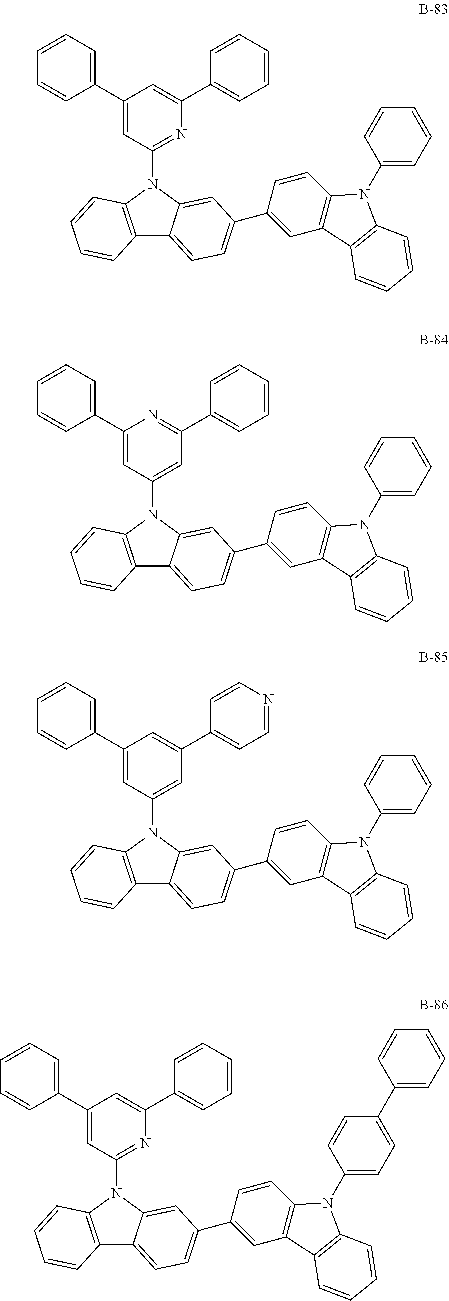 [SMILES: c1ccc(-c2cc(-c3ccccc3)nc(-n3c4ccccc4c4ccc(-c5ccc6c(c5)c5ccccc5n6-c5ccccc5)cc43)c2)cc1 ; c1ccc(-c2cc(-n3c4ccccc4c4ccc(-c5ccc6c(c5)c5ccccc5n6-c5ccccc5)cc43)cc(-c3ccccc3)n2)cc1 ; c1ccc(-c2cc(-c3ccncc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1]

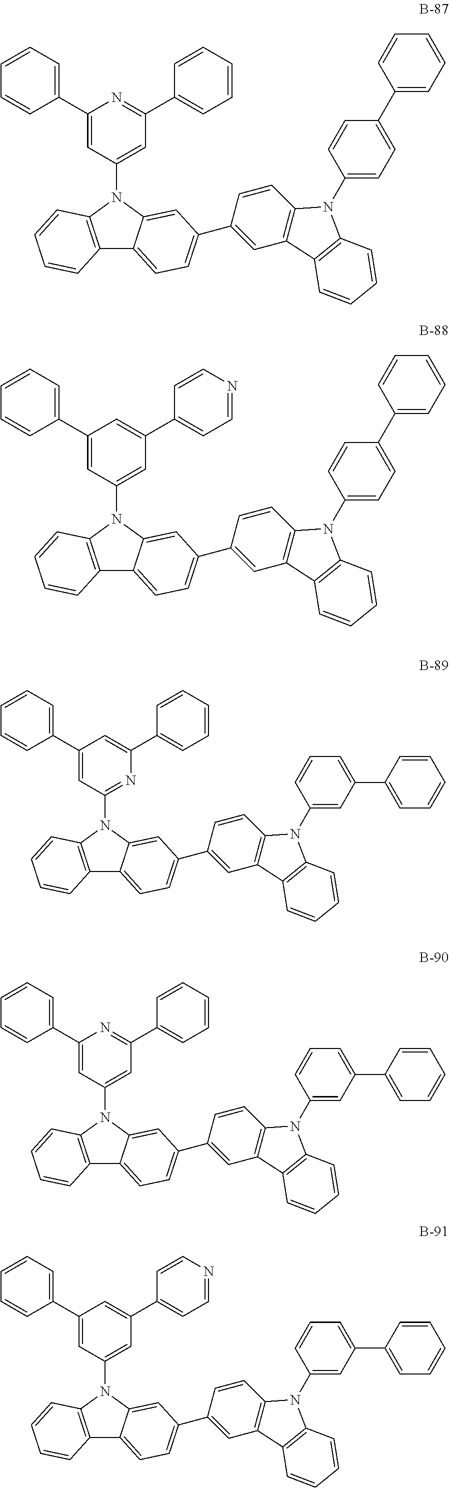 [SMILES: c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1 ; c1ccc(-c2ccc(-n3c4ccccc4c4cc(-c5ccc6c7ccccc7n(-c7cc(-c8ccccc8)cc(-c8ccncc8)c7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1 ; c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1 ; c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1]

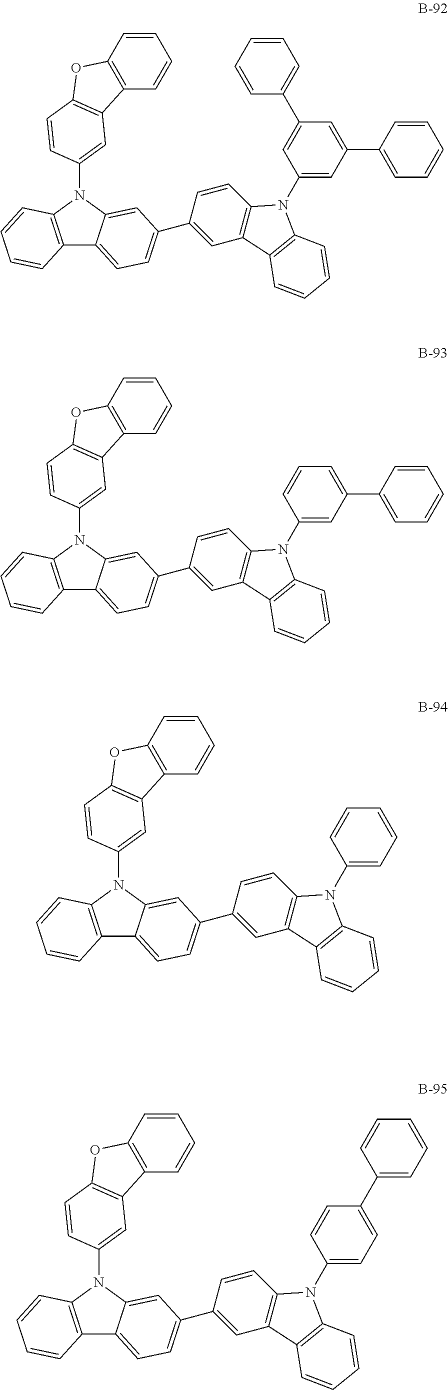 [SMILES: c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1 ; c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1 ; c1ccc(-c2ccc(-n3c4ccccc4c4cc(-c5ccc6c7ccccc7n(-c7ccc8oc9ccccc9c8c7)c6c5)ccc43)cc2)cc1]

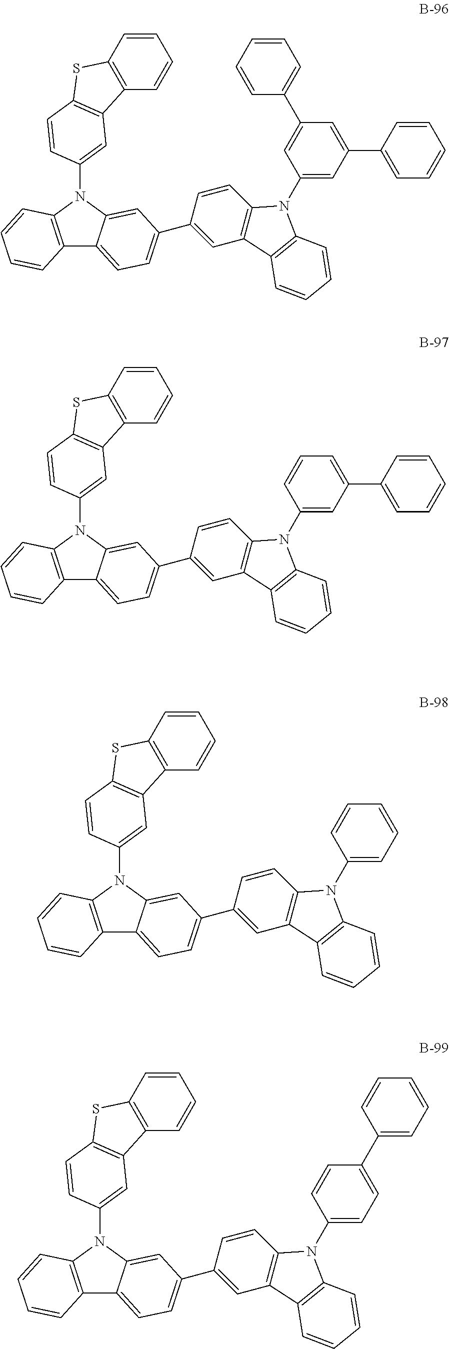 [SMILES: c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1 ; c1ccc(-c2cccc(-n3c4ccccc4c4cc(-c5ccc6c7ccccc7n(-c7ccc8sc9ccccc9c8c7)c6c5)ccc43)c2)cc1 ; c1ccc(-n2c3ccccc3c3cc(-c4ccc5c6ccccc6n(-c6ccc7sc8ccccc8c7c6)c5c4)ccc32)cc1 ; c1ccc(-c2ccc(-n3c4ccccc4c4cc(-c5ccc6c7ccccc7n(-c7ccc8sc9ccccc9c8c7)c6c5)ccc43)cc2)cc1]

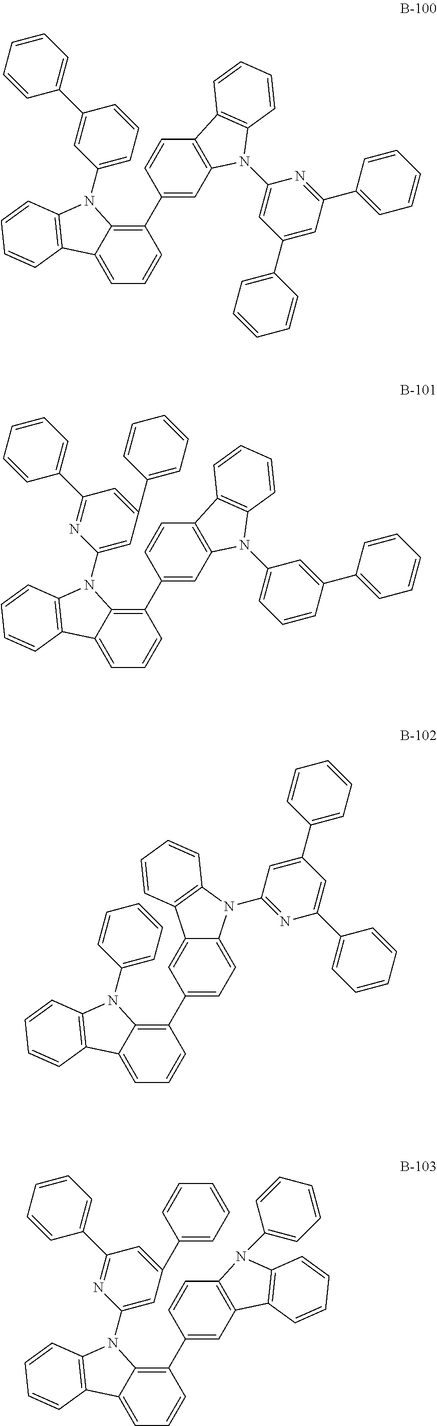 [SMILES: c1ccc(-c2cccc(-n3c4ccccc4c4cccc(-c5ccc6c7ccccc7n(-c7cc(-c8ccccc8)cc(-c8ccccc8)n7)c6c5)c43)c2)cc1 ; c1ccc(-c2cccc(-n3c4ccccc4c4ccc(-c5cccc6c7ccccc7n(-c7cc(-c8ccccc8)cc(-c8ccccc8)n7)c56)cc43)c2)cc1 ; c1ccc(-c2cc(-c3ccccc3)nc(-n3c4ccccc4c4cc(-c5cccc6c7ccccc7n(-c7ccccc7)c56)ccc43)c2)cc1 ; c1ccc(-c2cc(-c3ccccc3)nc(-n3c4ccccc4c4cccc(-c5ccc6c(c5)c5ccccc5n6-c5ccccc5)c43)c2)cc1]

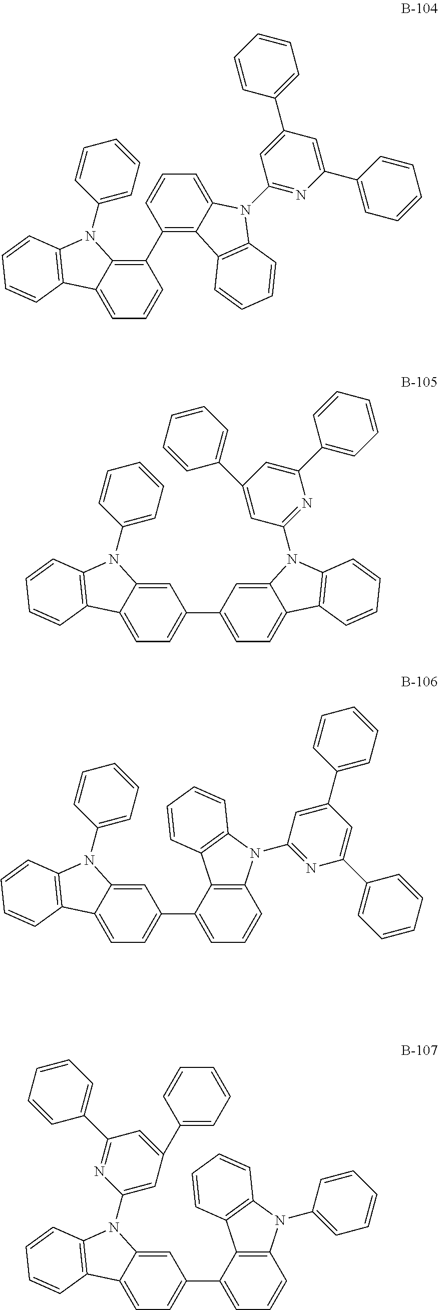 [SMILES: c1ccc(-c2cc(-c3ccccc3)nc(-n3c4ccccc4c4c(-c5cccc6c7ccccc7n(-c7ccccc7)c56)cccc43)c2)cc1 ; c1ccc(-c2cc(-c3ccccc3)nc(-n3c4ccccc4c4ccc(-c5ccc6c7ccccc7n(-c7ccccc7)c6c5)cc43)c2)cc1 ; c1ccc(-c2cc(-c3ccccc3)nc(-n3c4ccccc4c4c(-c5ccc6c7ccccc7n(-c7ccccc7)c6c5)cccc43)c2)cc1 ; c1ccc(-c2cc(-c3ccccc3)nc(-n3c4ccccc4c4ccc(-c5cccc6c5c5ccccc5n6-c5ccccc5)cc43)c2)cc1]

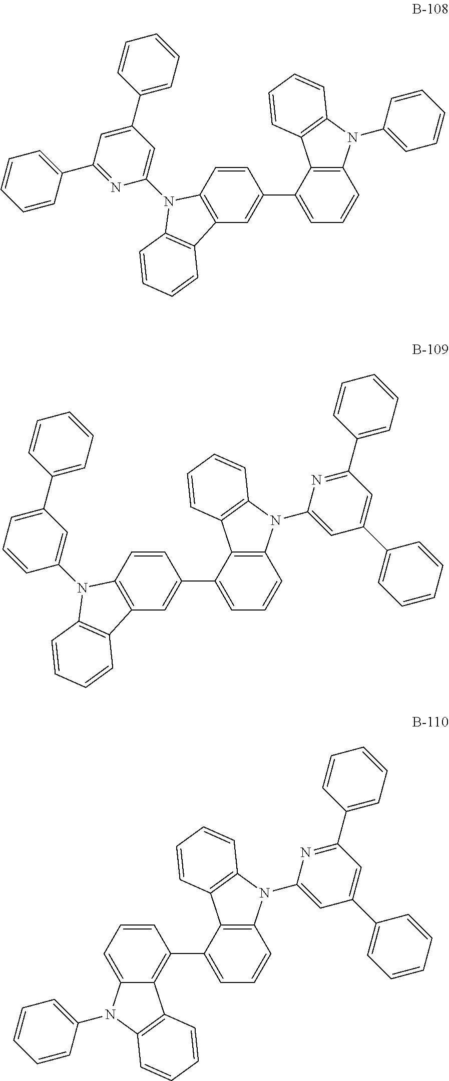 [SMILES: c1ccc(-c2cc(-c3ccccc3)nc(-n3c4ccccc4c4cc(-c5cccc6c5c5ccccc5n6-c5ccccc5)ccc43)c2)cc1 ; c1ccc(-c2cccc(-n3c4ccccc4c4cc(-c5cccc6c5c5ccccc5n6-c5cc(-c6ccccc6)cc(-c6ccccc6)n5)ccc43)c2)cc1 ; c1ccc(-c2cc(-c3ccccc3)nc(-n3c4ccccc4c4c(-c5cccc6c5c5ccccc5n6-c5ccccc5)cccc43)c2)cc1]

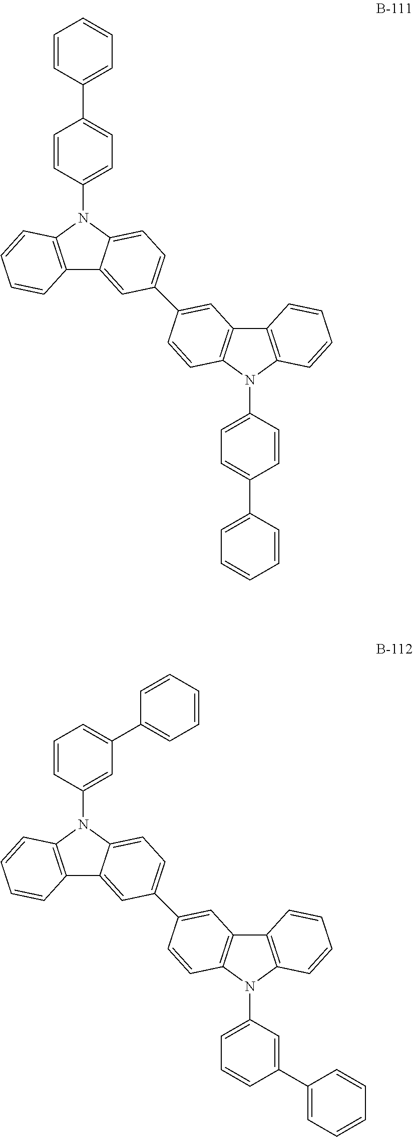 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

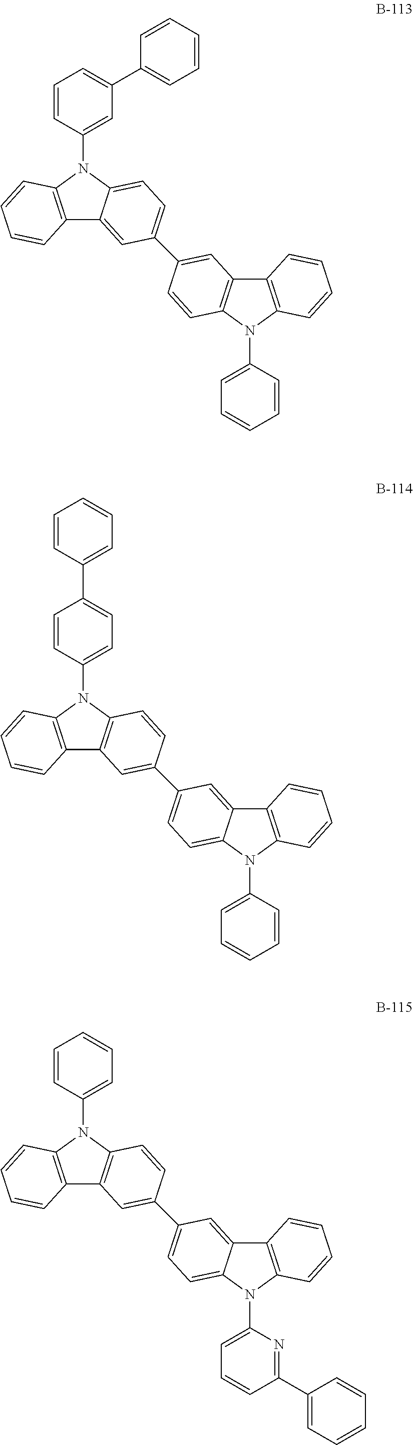 [SMILES: c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)n2)cc1]

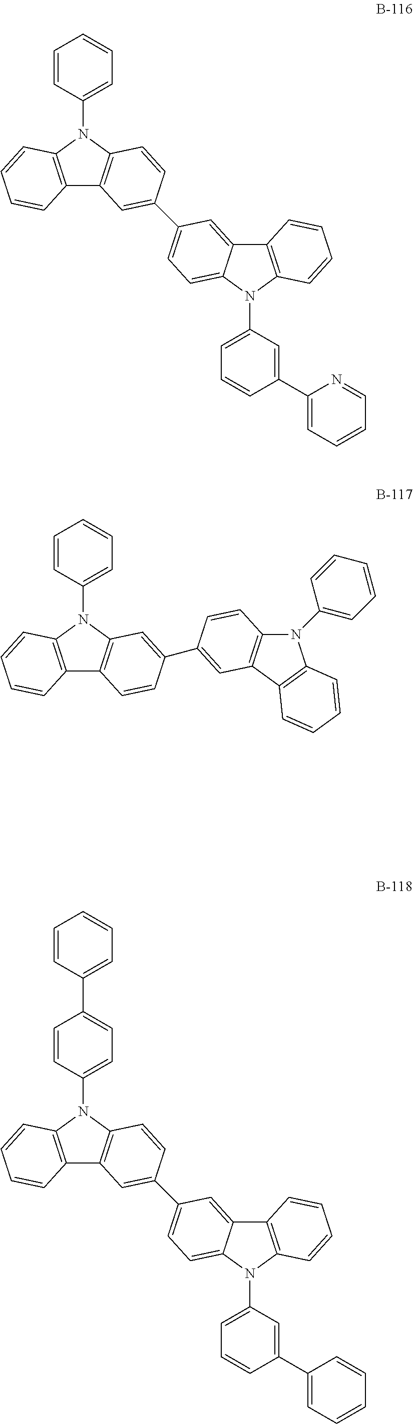 [SMILES: c1ccc(-n2c3ccccc3c3cc(-c4ccc5c(c4)c4ccccc4n5-c4cccc(-c5ccccn5)c4)ccc32)cc1 ; c1ccc(-n2c3ccccc3c3cc(-c4ccc5c6ccccc6n(-c6ccccc6)c5c4)ccc32)cc1 ; c1ccc(-c2ccc(-n3c4ccccc4c4cc(-c5ccc6c(c5)c5ccccc5n6-c5cccc(-c6ccccc6)c5)ccc43)cc2)cc1]

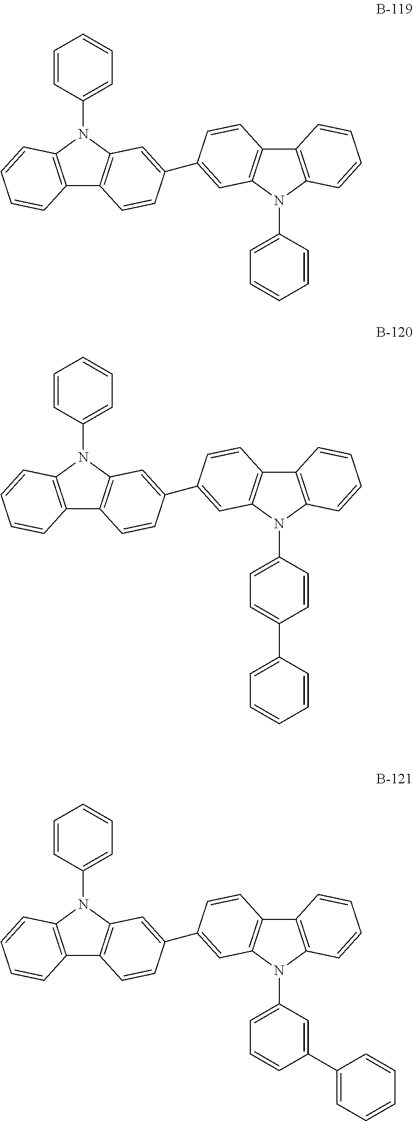 [SMILES: c1ccc(-n2c3ccccc3c3ccc(-c4ccc5c6ccccc6n(-c6ccccc6)c5c4)cc32)cc1 ; c1ccc(-c2ccc(-n3c4ccccc4c4ccc(-c5ccc6c7ccccc7n(-c7ccccc7)c6c5)cc43)cc2)cc1 ; c1ccc(-c2cccc(-n3c4ccccc4c4ccc(-c5ccc6c7ccccc7n(-c7ccccc7)c6c5)cc43)c2)cc1]

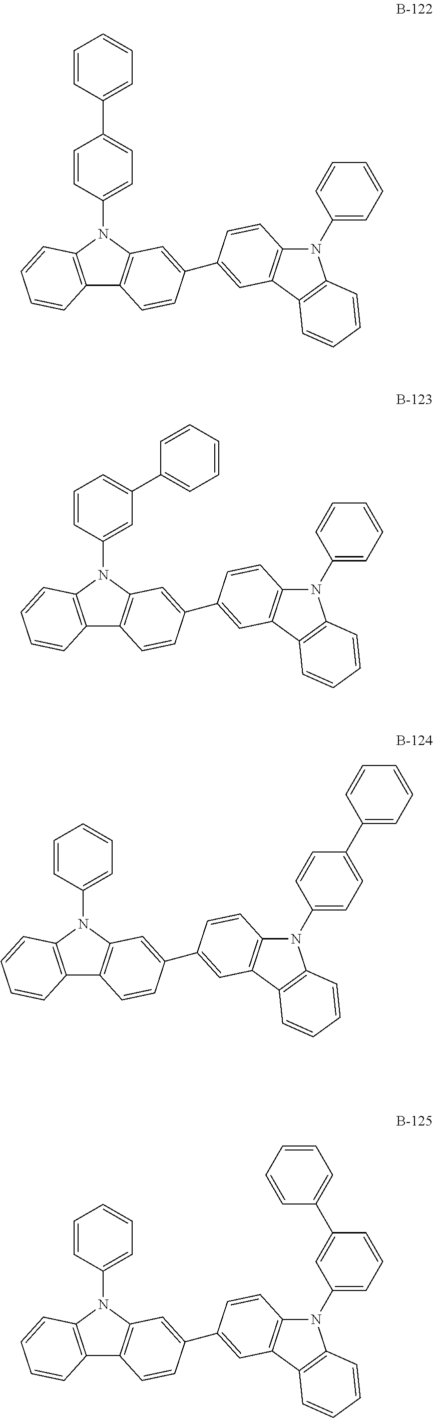 [SMILES: c1ccc(-c2ccc(-n3c4ccccc4c4ccc(-c5ccc6c(c5)c5ccccc5n6-c5ccccc5)cc43)cc2)cc1 ; c1ccc(-c2cccc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7ccccc7)c6c5)ccc43)cc2)cc1 ; c1ccc(-c2cccc(-n3c4ccccc4c4cc(-c5ccc6c7ccccc7n(-c7ccccc7)c6c5)ccc43)c2)cc1]

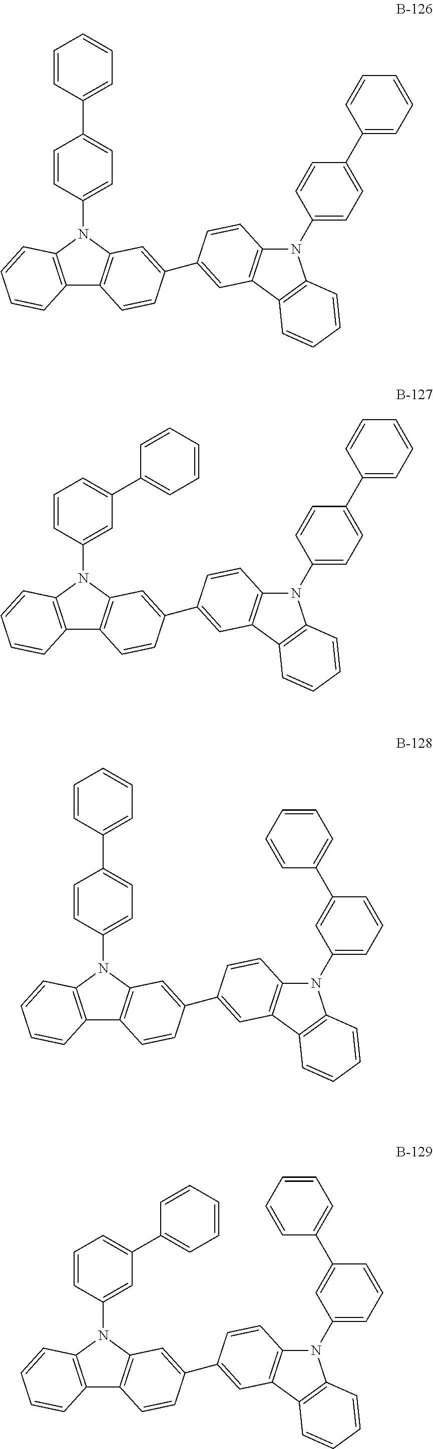 [SMILES: c1ccc(-c2ccc(-n3c4ccccc4c4cc(-c5ccc6c7ccccc7n(-c7ccc(-c8ccccc8)cc7)c6c5)ccc43)cc2)cc1 ; c1ccc(-c2ccc(-n3c4ccccc4c4cc(-c5ccc6c7ccccc7n(-c7cccc(-c8ccccc8)c7)c6c5)ccc43)cc2)cc1 ; c1ccc(-c2ccc(-n3c4ccccc4c4ccc(-c5ccc6c(c5)c5ccccc5n6-c5cccc(-c6ccccc6)c5)cc43)cc2)cc1 ; c1ccc(-c2cccc(-n3c4ccccc4c4cc(-c5ccc6c7ccccc7n(-c7cccc(-c8ccccc8)c7)c6c5)ccc43)c2)cc1]

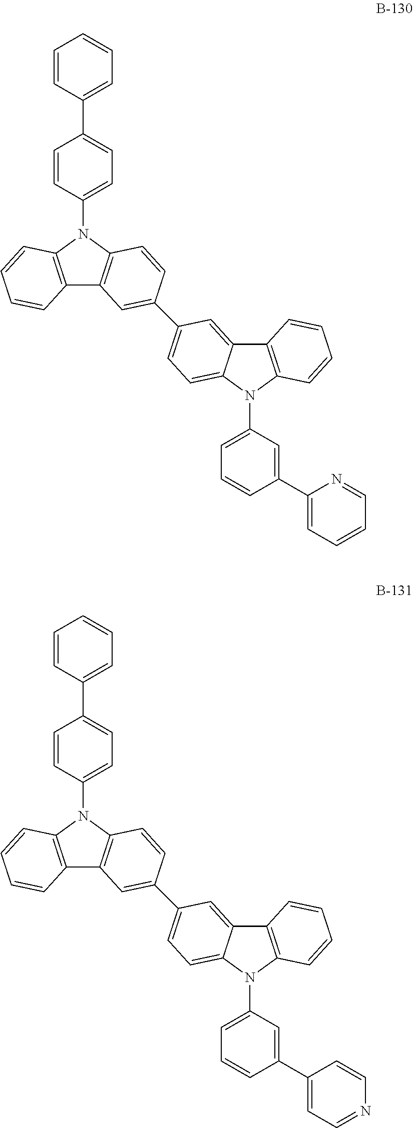 [SMILES: c1ccc(-c2ccc(-n3c4ccccc4c4cc(-c5ccc6c(c5)c5ccccc5n6-c5cccc(-c6ccccn6)c5)ccc43)cc2)cc1 ; c1ccc(-c2ccc(-n3c4ccccc4c4cc(-c5ccc6c(c5)c5ccccc5n6-c5cccc(-c6ccncc6)c5)ccc43)cc2)cc1]

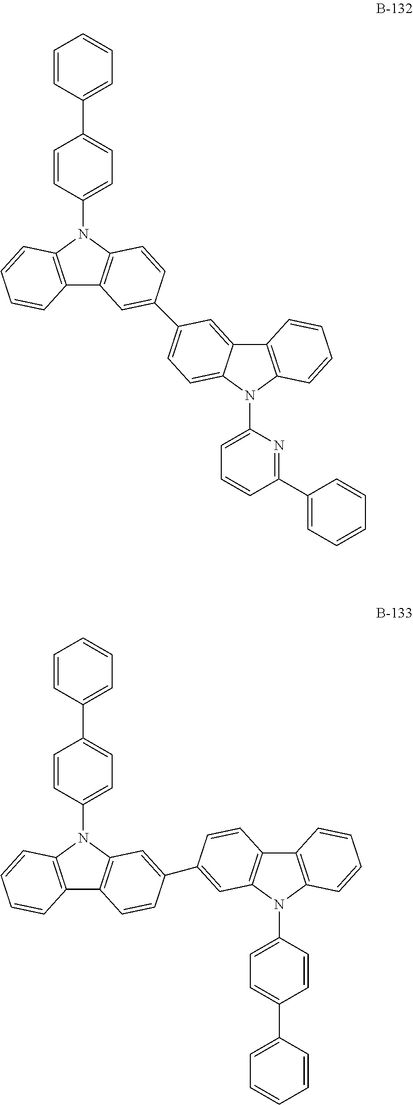 [SMILES: c1ccc(-c2ccc(-n3c4ccccc4c4cc(-c5ccc6c(c5)c5ccccc5n6-c5cccc(-c6ccccc6)n5)ccc43)cc2)cc1 ; c1ccc(-c2ccc(-n3c4ccccc4c4ccc(-c5ccc6c7ccccc7n(-c7ccc(-c8ccccc8)cc7)c6c5)cc43)cc2)cc1]

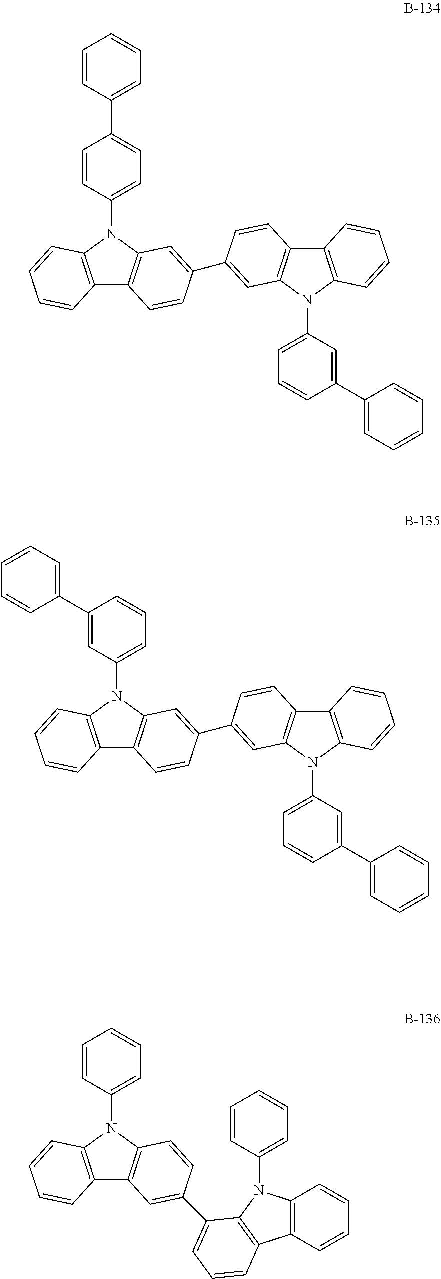 [SMILES: c1ccc(-c2ccc(-n3c4ccccc4c4ccc(-c5ccc6c7ccccc7n(-c7cccc(-c8ccccc8)c7)c6c5)cc43)cc2)cc1 ; c1ccc(-c2cccc(-n3c4ccccc4c4ccc(-c5ccc6c7ccccc7n(-c7cccc(-c8ccccc8)c7)c6c5)cc43)c2)cc1 ; c1ccc(-n2c3ccccc3c3cc(-c4cccc5c6ccccc6n(-c6ccccc6)c45)ccc32)cc1]

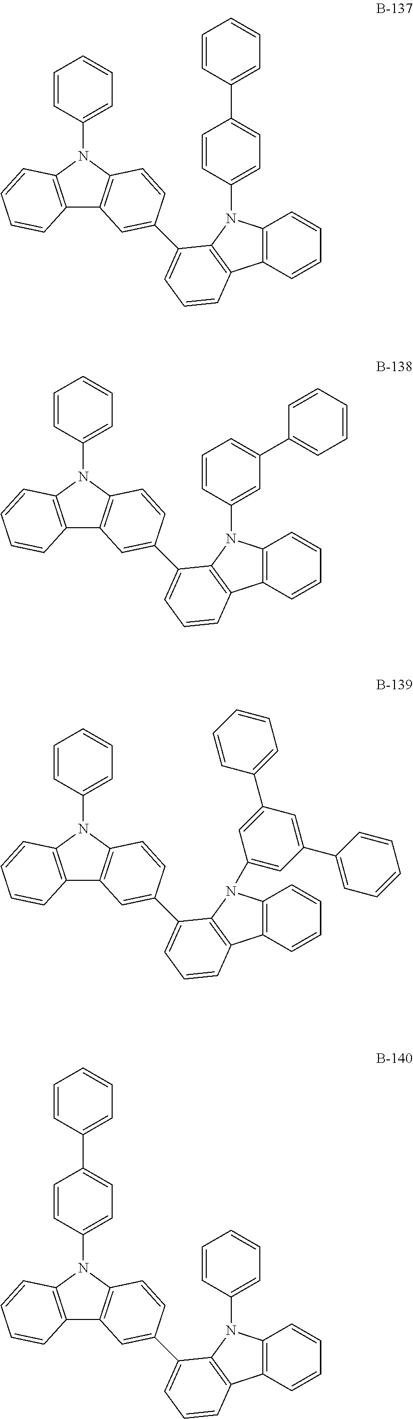 [SMILES: c1ccc(-c2ccc(-n3c4ccccc4c4cccc(-c5ccc6c(c5)c5ccccc5n6-c5ccccc5)c43)cc2)cc1 ; c1ccc(-c2cccc(-n3c4ccccc4c4cccc(-c5ccc6c(c5)c5ccccc5n6-c5ccccc5)c43)c2)cc1 ; c1ccc(-c2cc(-c3ccccc3)cc(-n3c4ccccc4c4cccc(-c5ccc6c(c5)c5ccccc5n6-c5ccccc5)c43)c2)cc1 ; c1ccc(-c2ccc(-n3c4ccccc4c4cc(-c5cccc6c7ccccc7n(-c7ccccc7)c56)ccc43)cc2)cc1]

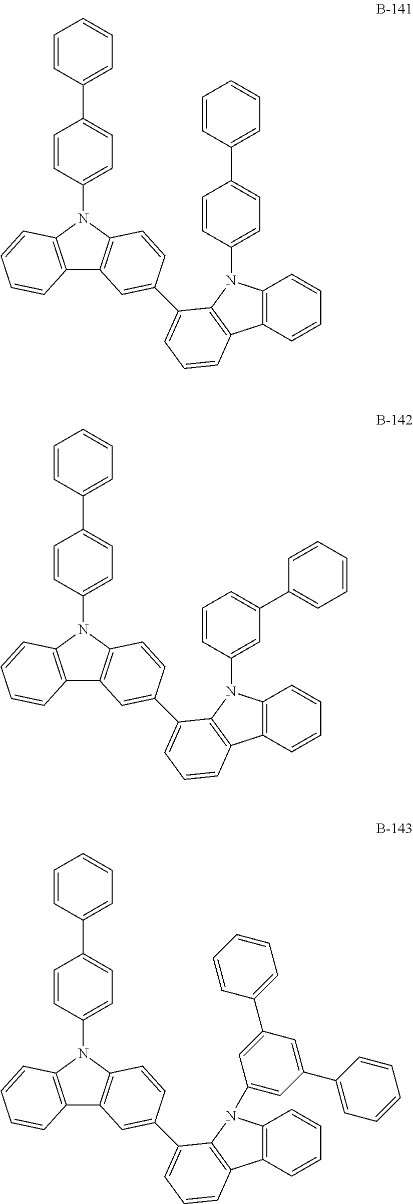 [SMILES: c1ccc(-c2ccc(-n3c4ccccc4c4cc(-c5cccc6c7ccccc7n(-c7ccc(-c8ccccc8)cc7)c56)ccc43)cc2)cc1 ; c1ccc(-c2ccc(-n3c4ccccc4c4cc(-c5cccc6c7ccccc7n(-c7cccc(-c8ccccc8)c7)c56)ccc43)cc2)cc1 ; c1ccc(-c2ccc(-n3c4ccccc4c4cc(-c5cccc6c7ccccc7n(-c7cc(-c8ccccc8)cc(-c8ccccc8)c7)c56)ccc43)cc2)cc1]

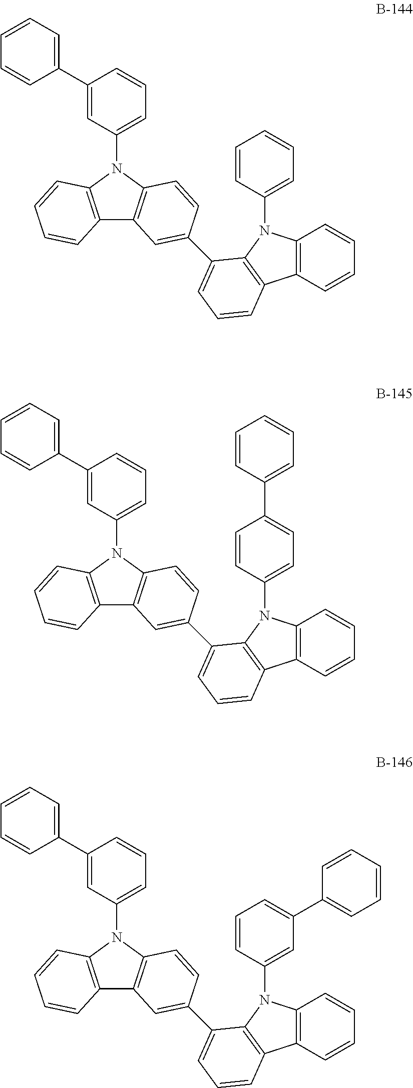 [SMILES: c1ccc(-c2cccc(-n3c4ccccc4c4cc(-c5cccc6c7ccccc7n(-c7ccccc7)c56)ccc43)c2)cc1 ; c1ccc(-c2ccc(-n3c4ccccc4c4cccc(-c5ccc6c(c5)c5ccccc5n6-c5cccc(-c6ccccc6)c5)c43)cc2)cc1 ; c1ccc(-c2cccc(-n3c4ccccc4c4cc(-c5cccc6c7ccccc7n(-c7cccc(-c8ccccc8)c7)c56)ccc43)c2)cc1]

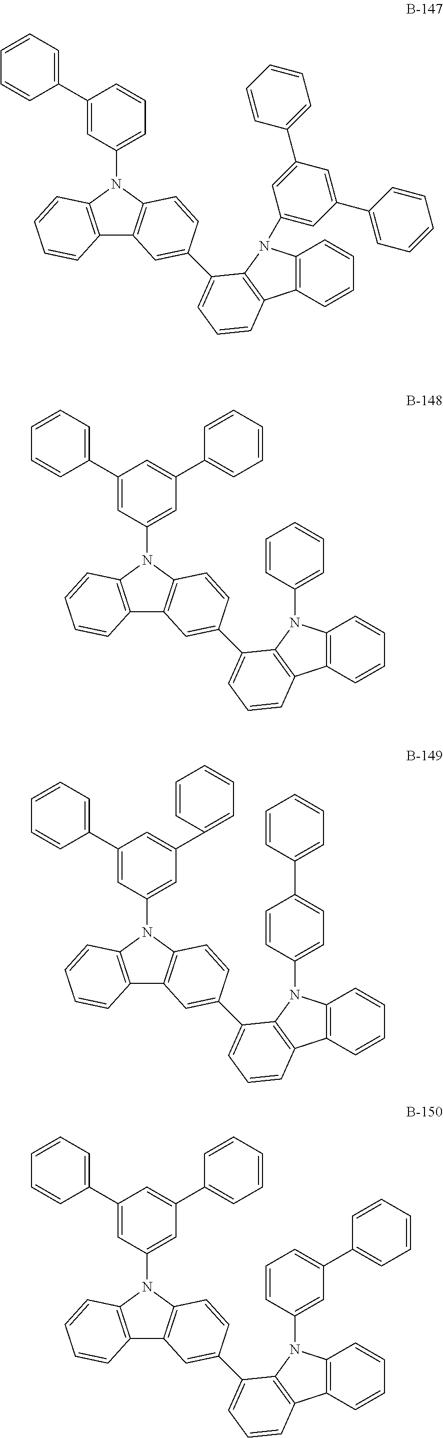 [SMILES: c1ccc(-c2cccc(-n3c4ccccc4c4cc(-c5cccc6c7ccccc7n(-c7cc(-c8ccccc8)cc(-c8ccccc8)c7)c56)ccc43)c2)cc1 ; c1ccc(-c2cc(-c3ccccc3)cc(-n3c4ccccc4c4cc(-c5cccc6c7ccccc7n(-c7ccccc7)c56)ccc43)c2)cc1 ; c1ccc(-c2ccc(-n3c4ccccc4c4cccc(-c5ccc6c(c5)c5ccccc5n6-c5cc(-c6ccccc6)cc(-c6ccccc6)c5)c43)cc2)cc1 ; c1ccc(-c2cc(-c3ccccc3)cc(-n3c4ccccc4c4cc(-c5cccc6c7ccccc7n(-c7cccc(-c8ccccc8)c7)c56)ccc43)c2)cc1]

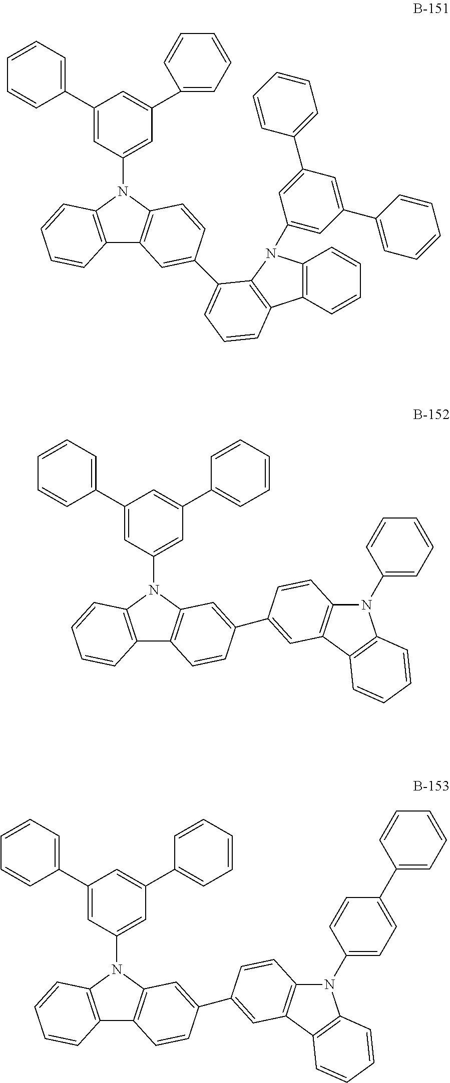 [SMILES: c1ccc(-c2cc(-c3ccccc3)cc(-n3c4ccccc4c4cc(-c5cccc6c7ccccc7n(-c7cc(-c8ccccc8)cc(-c8ccccc8)c7)c56)ccc43)c2)cc1 ; c1ccc(-c2cc(-c3ccccc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)c7)c6c5)ccc43)cc2)cc1]

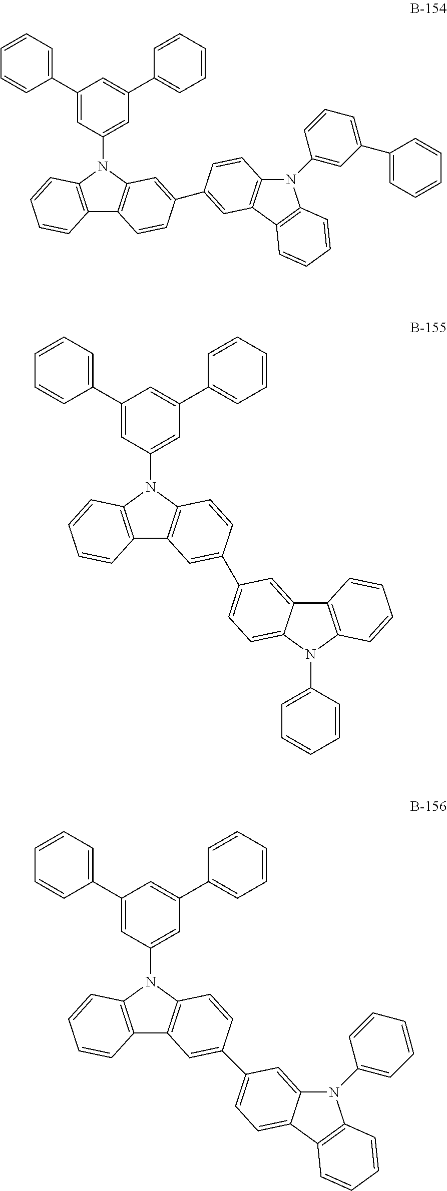 [SMILES: c1ccc(-c2cccc(-n3c4ccccc4c4cc(-c5ccc6c7ccccc7n(-c7cc(-c8ccccc8)cc(-c8ccccc8)c7)c6c5)ccc43)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cc(-c3ccccc3)cc(-n3c4ccccc4c4cc(-c5ccc6c7ccccc7n(-c7ccccc7)c6c5)ccc43)c2)cc1]

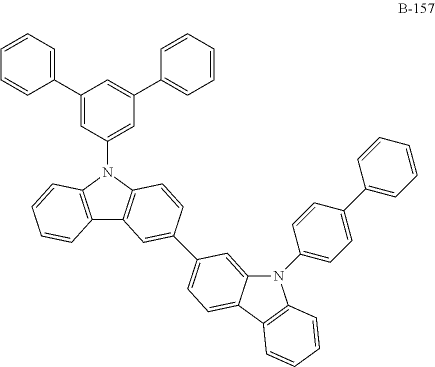 [SMILES: c1ccc(-c2ccc(-n3c4ccccc4c4ccc(-c5ccc6c(c5)c5ccccc5n6-c5cc(-c6ccccc6)cc(-c6ccccc6)c5)cc43)cc2)cc1]

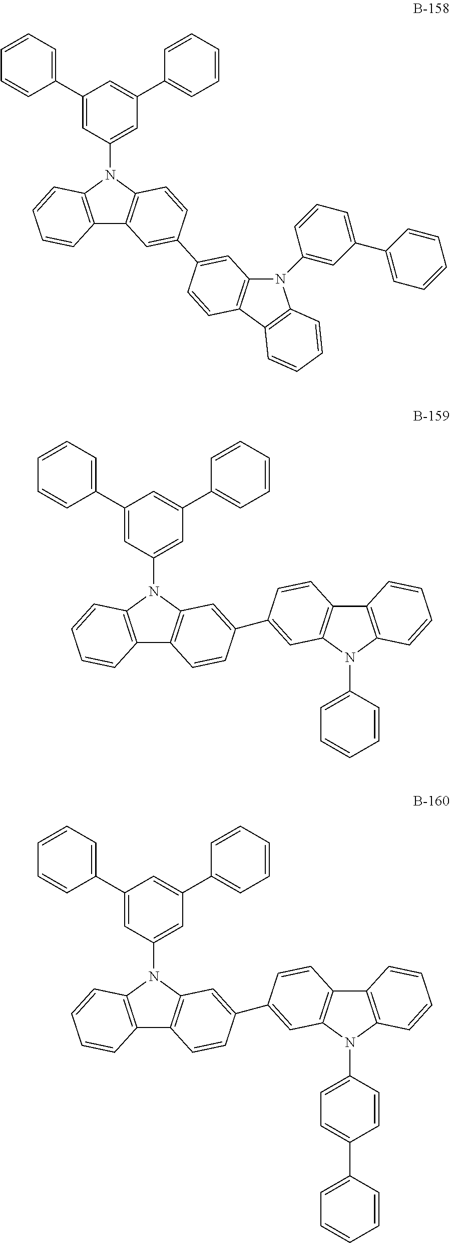 [SMILES: c1ccc(-c2cccc(-n3c4ccccc4c4ccc(-c5ccc6c(c5)c5ccccc5n6-c5cc(-c6ccccc6)cc(-c6ccccc6)c5)cc43)c2)cc1 ; c1ccc(-c2cc(-c3ccccc3)cc(-n3c4ccccc4c4ccc(-c5ccc6c7ccccc7n(-c7ccccc7)c6c5)cc43)c2)cc1 ; c1ccc(-c2ccc(-n3c4ccccc4c4ccc(-c5ccc6c7ccccc7n(-c7cc(-c8ccccc8)cc(-c8ccccc8)c7)c6c5)cc43)cc2)cc1]

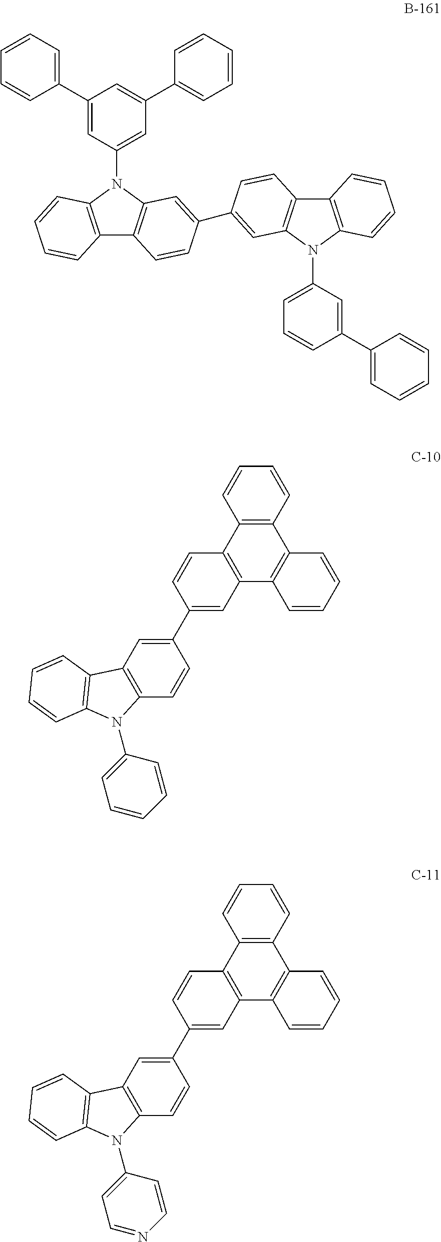 [SMILES: c1ccc(-c2cccc(-n3c4ccccc4c4ccc(-c5ccc6c7ccccc7n(-c7cc(-c8ccccc8)cc(-c8ccccc8)c7)c6c5)cc43)c2)cc1 ; c1ccc(-n2c3ccccc3c3cc(-c4ccc5c6ccccc6c6ccccc6c5c4)ccc32)cc1 ; c1ccc2c(c1)c1ccccc1c1cc(-c3ccc4c(c3)c3ccccc3n4-c3ccncc3)ccc21]

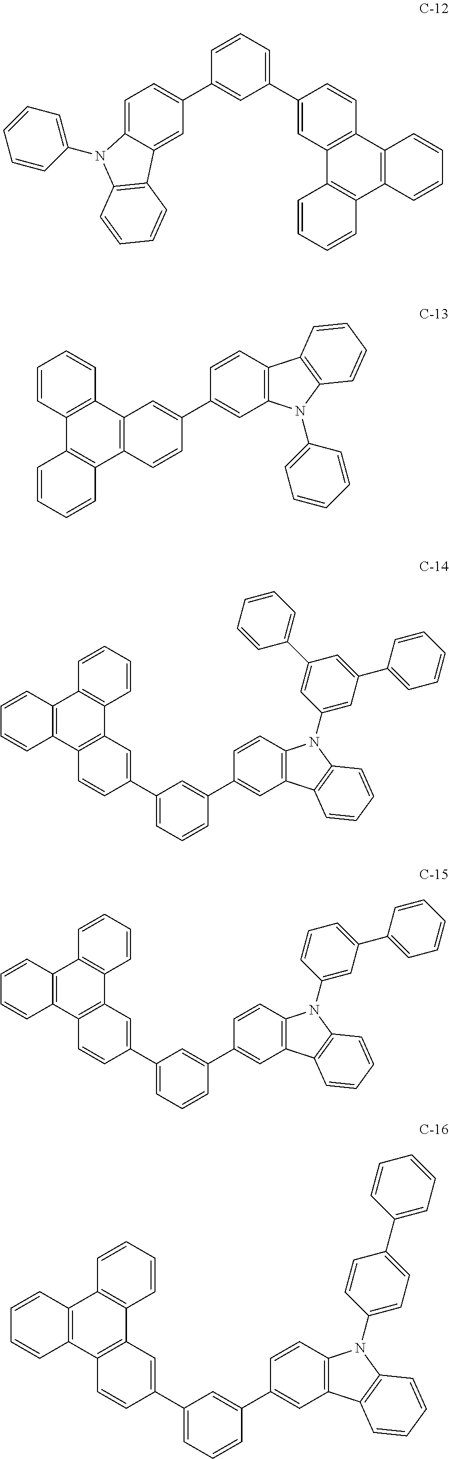 [SMILES: c1ccc(-n2c3ccccc3c3cc(-c4cccc(-c5ccc6c7ccccc7c7ccccc7c6c5)c4)ccc32)cc1 ; c1ccc(-n2c3ccccc3c3ccc(-c4ccc5c6ccccc6c6ccccc6c5c4)cc32)cc1 ; c1ccc(-c2cc(-c3ccccc3)cc(-n3c4ccccc4c4cc(-c5cccc(-c6ccc7c8ccccc8c8ccccc8c7c6)c5)ccc43)c2)cc1 ; c1ccc(-c2cccc(-n3c4ccccc4c4cc(-c5cccc(-c6ccc7c8ccccc8c8ccccc8c7c6)c5)ccc43)c2)cc1 ; c1ccc(-c2ccc(-n3c4ccccc4c4cc(-c5cccc(-c6ccc7c8ccccc8c8ccccc8c7c6)c5)ccc43)cc2)cc1]

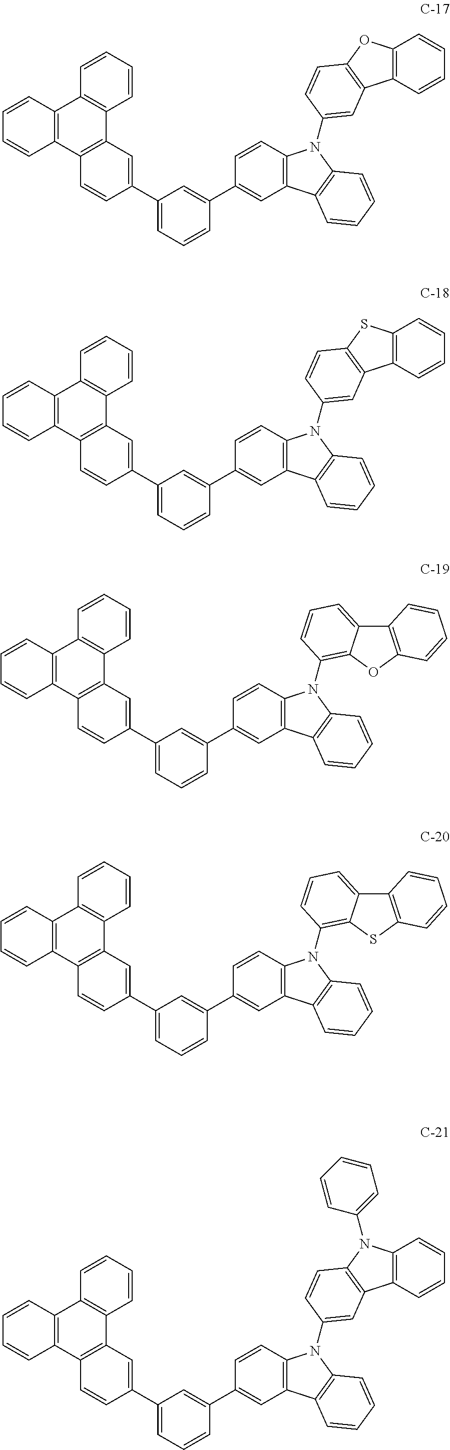 [SMILES: c1cc(-c2ccc3c4ccccc4c4ccccc4c3c2)cc(-c2ccc3c(c2)c2ccccc2n3-c2ccc3oc4ccccc4c3c2)c1 ; c1cc(-c2ccc3c4ccccc4c4ccccc4c3c2)cc(-c2ccc3c(c2)c2ccccc2n3-c2ccc3sc4ccccc4c3c2)c1 ; c1cc(-c2ccc3c4ccccc4c4ccccc4c3c2)cc(-c2ccc3c(c2)c2ccccc2n3-c2cccc3c2oc2ccccc23)c1 ; c1cc(-c2ccc3c4ccccc4c4ccccc4c3c2)cc(-c2ccc3c(c2)c2ccccc2n3-c2cccc3c2sc2ccccc23)c1 ; c1ccc(-n2c3ccccc3c3cc(-n4c5ccccc5c5cc(-c6cccc(-c7ccc8c9ccccc9c9ccccc9c8c7)c6)ccc54)ccc32)cc1]

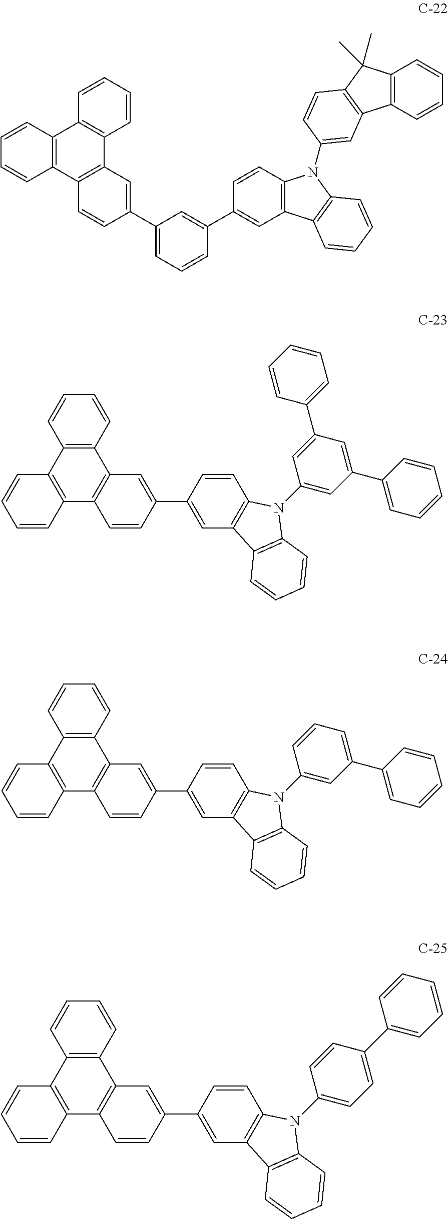 [SMILES: CC1(C)c2ccccc2-c2cc(-n3c4ccccc4c4cc(-c5cccc(-c6ccc7c8ccccc8c8ccccc8c7c6)c5)ccc43)ccc21 ; c1ccc(-c2cc(-c3ccccc3)cc(-n3c4ccccc4c4cc(-c5ccc6c7ccccc7c7ccccc7c6c5)ccc43)c2)cc1 ; c1ccc(-c2cccc(-n3c4ccccc4c4cc(-c5ccc6c7ccccc7c7ccccc7c6c5)ccc43)c2)cc1 ; c1ccc(-c2ccc(-n3c4ccccc4c4cc(-c5ccc6c7ccccc7c7ccccc7c6c5)ccc43)cc2)cc1]

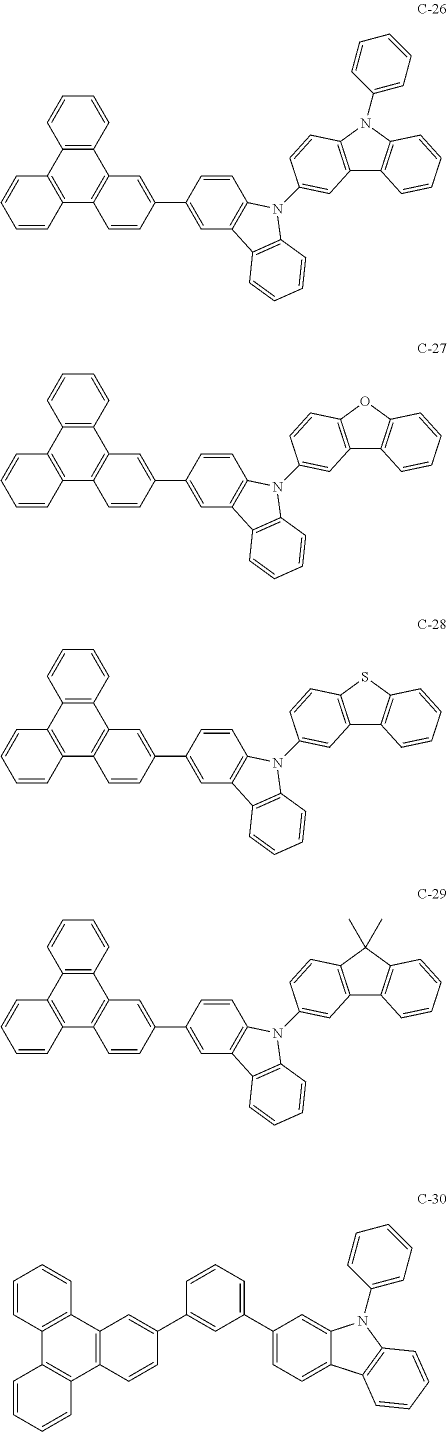 [SMILES: c1ccc(-n2c3ccccc3c3cc(-n4c5ccccc5c5cc(-c6ccc7c8ccccc8c8ccccc8c7c6)ccc54)ccc32)cc1 ; c1ccc2c(c1)oc1ccc(-n3c4ccccc4c4cc(-c5ccc6c7ccccc7c7ccccc7c6c5)ccc43)cc12 ; c1ccc2c(c1)sc1ccc(-n3c4ccccc4c4cc(-c5ccc6c7ccccc7c7ccccc7c6c5)ccc43)cc12 ; CC1(C)c2ccccc2-c2cc(-n3c4ccccc4c4cc(-c5ccc6c7ccccc7c7ccccc7c6c5)ccc43)ccc21 ; c1ccc(-n2c3ccccc3c3ccc(-c4cccc(-c5ccc6c7ccccc7c7ccccc7c6c5)c4)cc32)cc1]

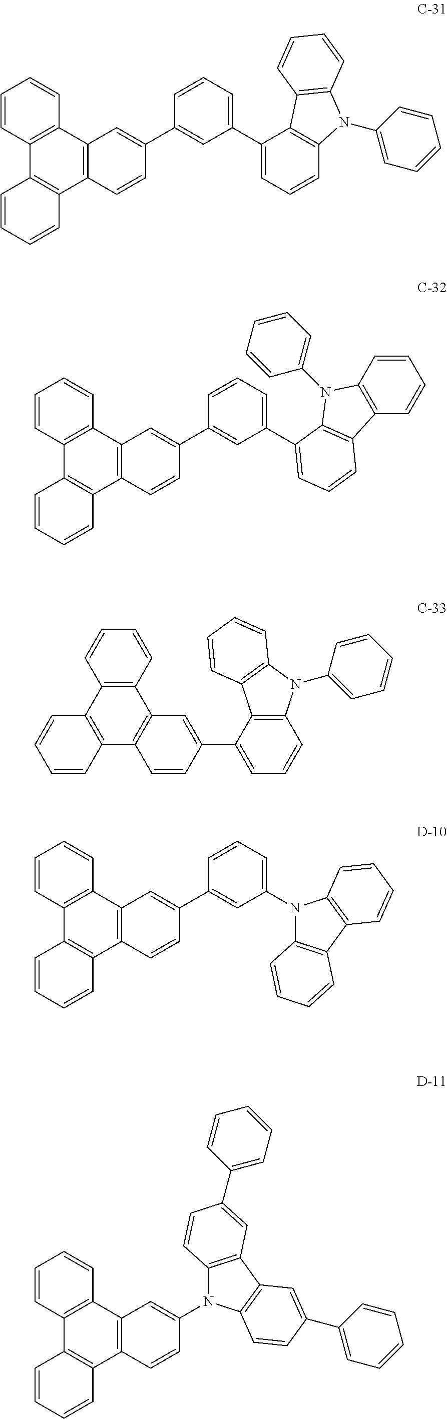 [SMILES: c1ccc(-n2c3ccccc3c3c(-c4cccc(-c5ccc6c7ccccc7c7ccccc7c6c5)c4)cccc32)cc1 ; c1ccc(-n2c3ccccc3c3cccc(-c4cccc(-c5ccc6c7ccccc7c7ccccc7c6c5)c4)c32)cc1 ; c1ccc(-n2c3ccccc3c3c(-c4ccc5c6ccccc6c6ccccc6c5c4)cccc32)cc1 ; c1cc(-c2ccc3c4ccccc4c4ccccc4c3c2)cc(-n2c3ccccc3c3ccccc32)c1 ; c1ccc(-c2ccc3c(c2)c2cc(-c4ccccc4)ccc2n3-c2ccc3c4ccccc4c4ccccc4c3c2)cc1]

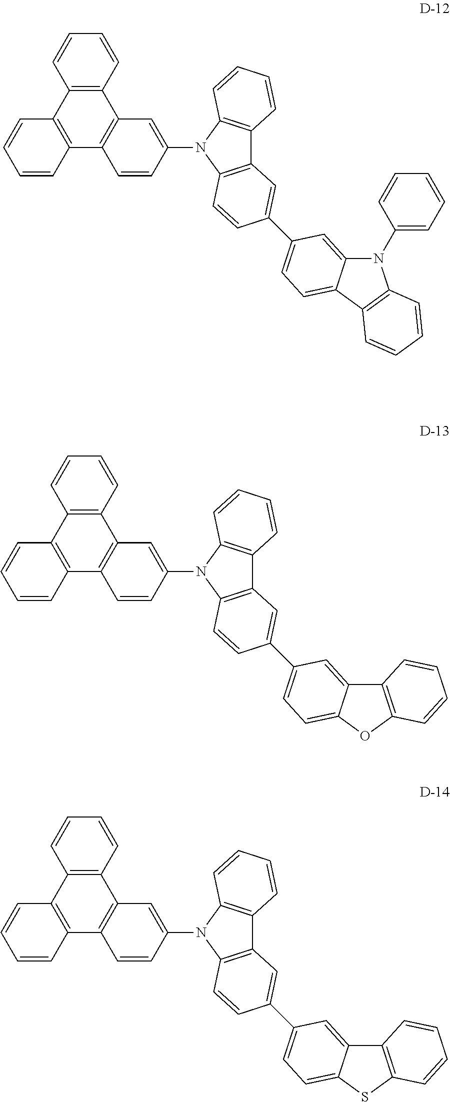 [SMILES: c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)oc1ccc(-c3ccc4c(c3)c3ccccc3n4-c3ccc4c5ccccc5c5ccccc5c4c3)cc12 ; c1ccc2c(c1)sc1ccc(-c3ccc4c(c3)c3ccccc3n4-c3ccc4c5ccccc5c5ccccc5c4c3)cc12]

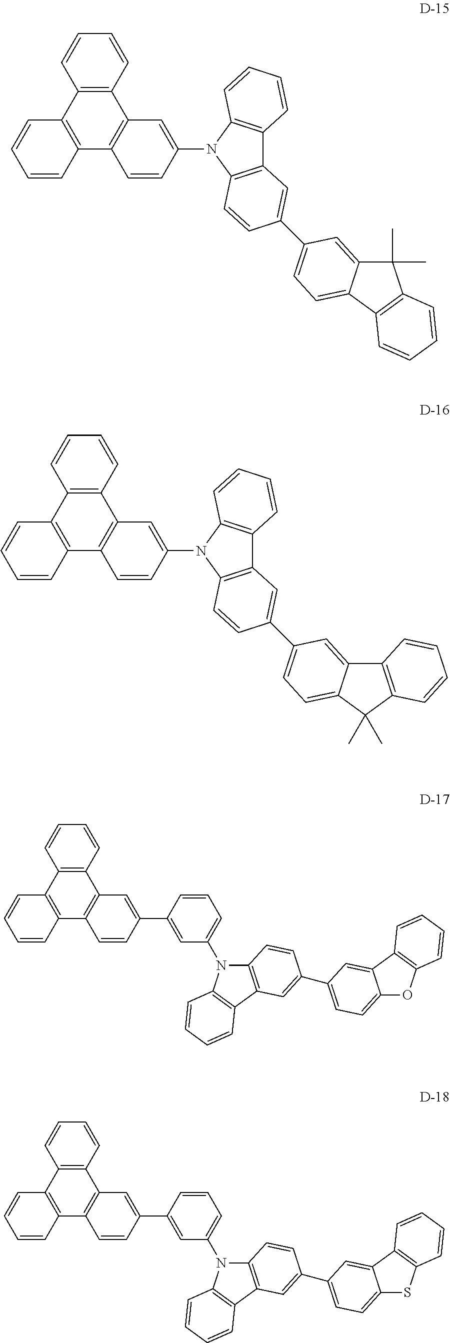 [SMILES: CC1(C)c2ccccc2-c2ccc(-c3ccc4c(c3)c3ccccc3n4-c3ccc4c5ccccc5c5ccccc5c4c3)cc21 ; CC1(C)c2ccccc2-c2cc(-c3ccc4c(c3)c3ccccc3n4-c3ccc4c5ccccc5c5ccccc5c4c3)ccc21 ; c1cc(-c2ccc3c4ccccc4c4ccccc4c3c2)cc(-n2c3ccccc3c3cc(-c4ccc5oc6ccccc6c5c4)ccc32)c1 ; c1cc(-c2ccc3c4ccccc4c4ccccc4c3c2)cc(-n2c3ccccc3c3cc(-c4ccc5sc6ccccc6c5c4)ccc32)c1]

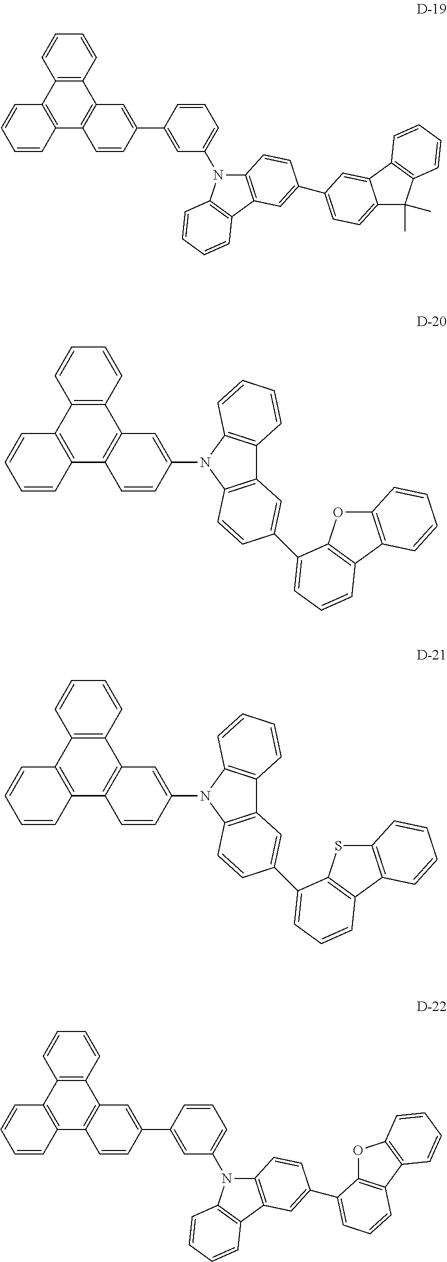 [SMILES: CC1(C)c2ccccc2-c2cc(-c3ccc4c(c3)c3ccccc3n4-c3cccc(-c4ccc5c6ccccc6c6ccccc6c5c4)c3)ccc21 ; c1ccc2c(c1)oc1c(-c3ccc4c(c3)c3ccccc3n4-c3ccc4c5ccccc5c5ccccc5c4c3)cccc12 ; c1ccc2c(c1)sc1c(-c3ccc4c(c3)c3ccccc3n4-c3ccc4c5ccccc5c5ccccc5c4c3)cccc12 ; c1cc(-c2ccc3c4ccccc4c4ccccc4c3c2)cc(-n2c3ccccc3c3cc(-c4cccc5c4oc4ccccc45)ccc32)c1]

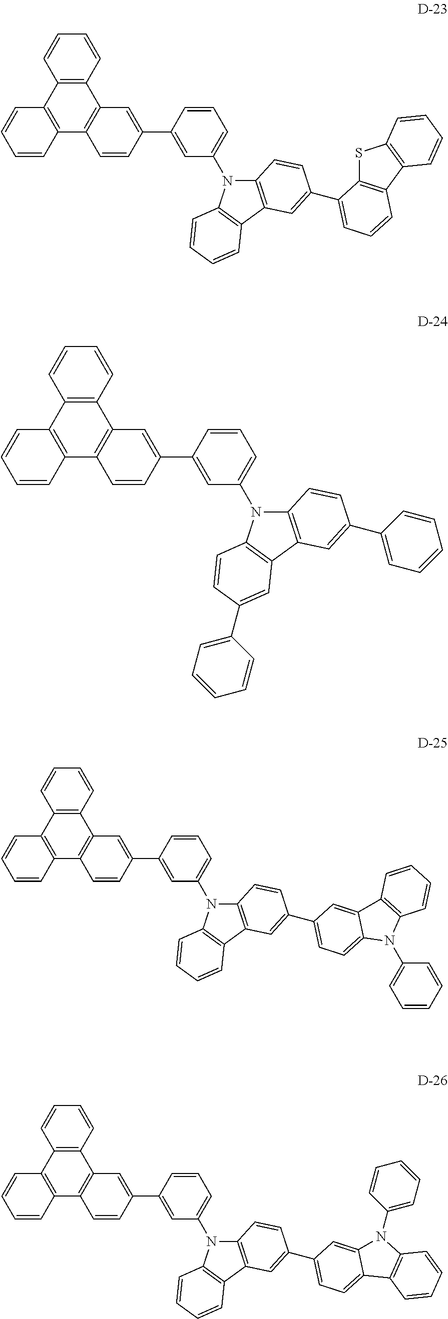 [SMILES: c1cc(-c2ccc3c4ccccc4c4ccccc4c3c2)cc(-n2c3ccccc3c3cc(-c4cccc5c4sc4ccccc45)ccc32)c1 ; c1ccc(-c2ccc3c(c2)c2cc(-c4ccccc4)ccc2n3-c2cccc(-c3ccc4c5ccccc5c5ccccc5c4c3)c2)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1ccc(-n2c3ccccc3c3ccc(-c4ccc5c(c4)c4ccccc4n5-c4cccc(-c5ccc6c7ccccc7c7ccccc7c6c5)c4)cc32)cc1]

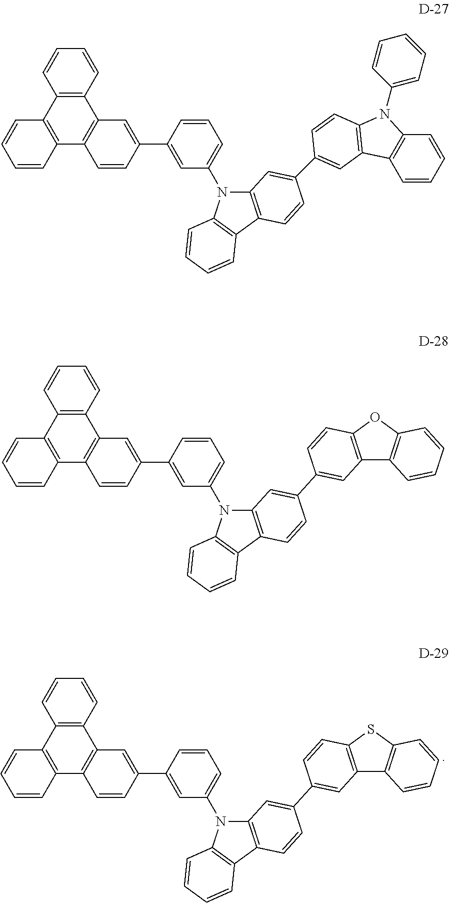 [SMILES: c1ccc(-n2c3ccccc3c3cc(-c4ccc5c6ccccc6n(-c6cccc(-c7ccc8c9ccccc9c9ccccc9c8c7)c6)c5c4)ccc32)cc1 ; c1cc(-c2ccc3c4ccccc4c4ccccc4c3c2)cc(-n2c3ccccc3c3ccc(-c4ccc5oc6ccccc6c5c4)cc32)c1 ; c1cc(-c2ccc3c4ccccc4c4ccccc4c3c2)cc(-n2c3ccccc3c3ccc(-c4ccc5sc6ccccc6c5c4)cc32)c1]

Synthesis methods of the organometallic compound represented by Formula 1 and the first to fourth compounds may be recognized by those of ordinary skill in the art by referring to Synthesis Examples to be described below.

### Organic Light-Emitting Device

The organometallic compound represented by Formula 1 may be suitable for use in an organic layer of an organic light-emitting device, for example, for use as a dopant in an emission layer of the organic layer, and at least one selected from the first to fourth compounds may be used as a host.

Therefore, in one or more embodiments, the emission layer of the organic light-emitting device may include a dopant and a host, the dopant may include one or more of organometallic compounds represented by Formula 1, and the host may include at least one selected from the first to fourth compounds.

The organic light-emitting device may have, due to the inclusion of an organic layer including the organometallic compound represented by Formula 1, a low driving voltage, high luminescent efficiency, high power, a long lifespan, and excellent color purity.

The expression that “(an organic layer) includes at least one of organometallic compounds” used herein may include a case in which “(an organic layer) includes identical organometallic compounds represented by Formula 1” and a case in which “(an organic layer) includes two or more different organometallic compounds represented by Formula 1.”

For example, the organic layer may include only Compound 1 as the organometallic compound. In this regard, Compound 1 may be included only in the emission layer of the organic light-emitting device. In one or more embodiments, the organic layer may include, as the organometallic compound, Compound 1 and Compound 2. In this regard, Compound 1 and Compound 2 may be included in an identical layer (for example, Compound 1 and Compound 2 all may exist in an emission layer).

The first electrode may be an anode, which is a hole injection electrode, and the second electrode may be a cathode, which is an electron injection electrode; or the first electrode may be a cathode, which is an electron injection electrode, and the second electrode may be an anode, which is a hole injection electrode.

In one or more embodiments, the first electrode may be an anode, the second electrode may be a cathode, the organic layer may include a hole transport region disposed between the first electrode and the emission layer and an electron transport region disposed between the emission layer and the second electrode, the hole transport region may include at least one layer selected from a hole injection layer, a hole transport layer, and an electron blocking layer, and the electron transport region may include at least one layer selected from a hole blocking layer, an electron transport layer, and an electron injection layer.

The term “organic layer” as used herein refers to a single layer and/or a multi-layer disposed between the first electrode and the second electrode in the organic light-emitting device. In one or more embodiments, the “organic layer” may include, in addition to an organic compound, an organometallic complex including metal.

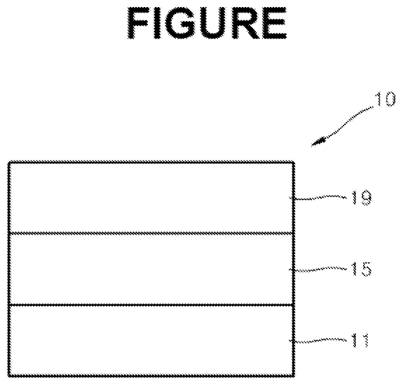

FIG. 1 is a schematic view of an organic light-emitting device 10 according to an embodiment. Hereinafter, the structure of an organic light-emitting device according to an embodiment and a method of manufacturing an organic light-emitting device according to an embodiment will be described in connection with FIG. 1. The organic light-emitting device 10 includes a first electrode 11, an organic layer 15, and a second electrode 19, which are sequentially stacked.

A substrate may be additionally disposed under the first electrode 11 or above the second electrode 19. For use as the substrate, any substrate that is used in general organic light-emitting devices may be used, and the substrate may be a glass substrate or a transparent plastic substrate, each having excellent mechanical strength, thermal stability, transparency, surface smoothness, ease of handling, and water-resistance.

The first electrode 11 may be formed by depositing or sputtering a material for forming the first electrode 11 on the substrate. The first electrode 11 may be an anode. The material for forming the first electrode 11 may be selected from materials with a high work function to facilitate hole injection. The first electrode 11 may be a reflective electrode, a semi-transmissive electrode, or a transmissive electrode. The material for forming the first electrode may be, for example, indium tin oxide (ITO), indium zinc oxide (IZO), tin oxide (SnO₂), and zinc oxide (ZnO). In one or more embodiments, magnesium (Mg), aluminum (Al), aluminum-lithium (Al—Li), calcium (Ca), magnesium-indium (Mg—In), or magnesium-silver (Mg—Ag) may be used as the material for forming the first electrode.

The first electrode 11 may have a single-layered structure or a multi-layered structure including two or more layers. For example, the first electrode 11 may have a three-layered structure of ITO/Ag/ITO, but the structure of the first electrode 110 is not limited thereto.

The organic layer 15 is disposed on the first electrode 11.

The organic layer 15 may include a hole transport region, an emission layer, and an electron transport region.

The hole transport region may be disposed between the first electrode 11 and the emission layer.

The hole transport region may include at least one selected from a hole injection layer, a hole transport layer, an electron blocking layer, and a buffer layer.

The hole transport region may include only either a hole injection layer or a hole transport layer. In one or more embodiments, the hole transport region may have a hole injection layer/hole transport layer structure or a hole injection layer/hole transport layer/electron blocking layer structure, which are sequentially stacked in this stated order from the first electrode 11.

A hole injection layer may be formed on the first electrode 11 by using one or more suitable methods selected from vacuum deposition, spin coating, casting, or Langmuir-Blodgett (LB) deposition.

When a hole injection layer is formed by vacuum deposition, the deposition conditions may vary according to a material that is used to form the hole injection layer, and the structure and thermal characteristics of the hole injection layer. For example, the deposition conditions may include a deposition temperature of about 100 to about 500° C., a vacuum pressure of about 10′⁸ to about 10′³ torr, and a deposition rate of about 0.01 to about 100 Angstroms per second (Å/sec). However, the deposition conditions are not limited thereto.

When the hole injection layer is formed using spin coating, coating conditions may vary according to the material used to form the hole injection layer, and the structure and thermal properties of the hole injection layer. For example, a coating speed may be from about 2,000 revolutions per minute (rpm) to about 5,000 rpm, and a temperature at which a heat treatment is performed to remove a solvent after coating may be from about 80° C. to about 200° C. However, the coating conditions are not limited thereto.

Conditions for forming a hole transport layer and an electron blocking layer may be understood by referring to conditions for forming the hole injection layer.

The hole transport region may include at least one selected from m-MTDATA, TDATA, 2-TNATA, NPB, (3-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzene sulfonic acid (Pani/DBSA), poly(3,4-ethylenedioxythiophene)/poly(4-styrene sulfonate) (PEDOT/PSS), polyaniline/camphor sulfonic acid (Pani/CSA), polyaniline/poly(4-styrene sulfonate) (Pani/PSS), a compound represented by Formula 201 below, and a compound represented by Formula 202:

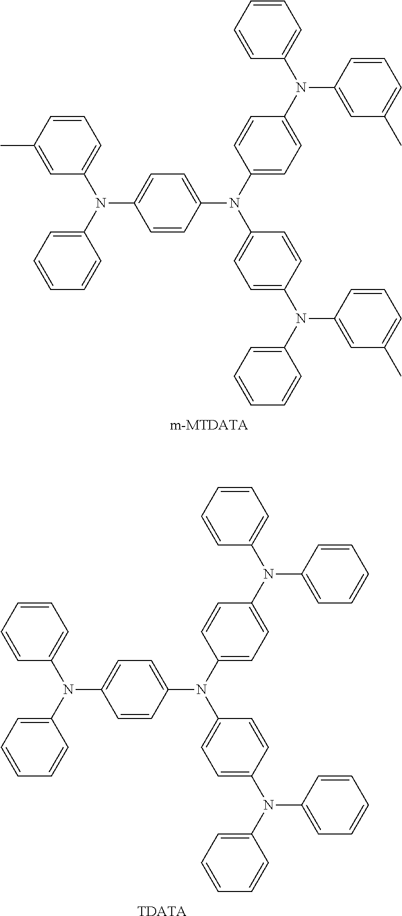

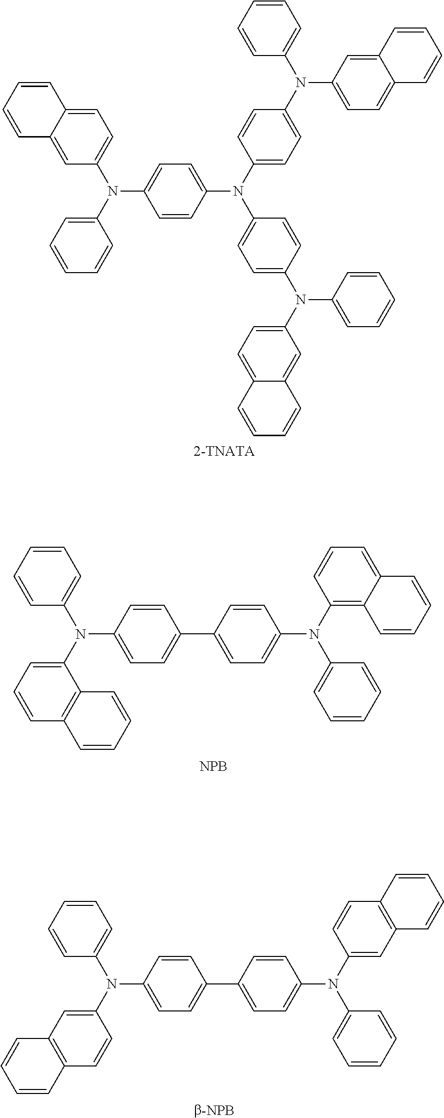 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; "2-TNATA" ; c1ccc(N(c2ccc(-c3ccc(N(c4ccccc4)c4cccc5ccccc45)cc3)cc2)c2cccc3ccccc23)cc1 ; "NPB" ; c1ccc(N(c2ccc(-c3ccc(N(c4ccccc4)c4ccc5ccccc5c4)cc3)cc2)c2ccc3ccccc3c2)cc1 ; "b-NPB"]

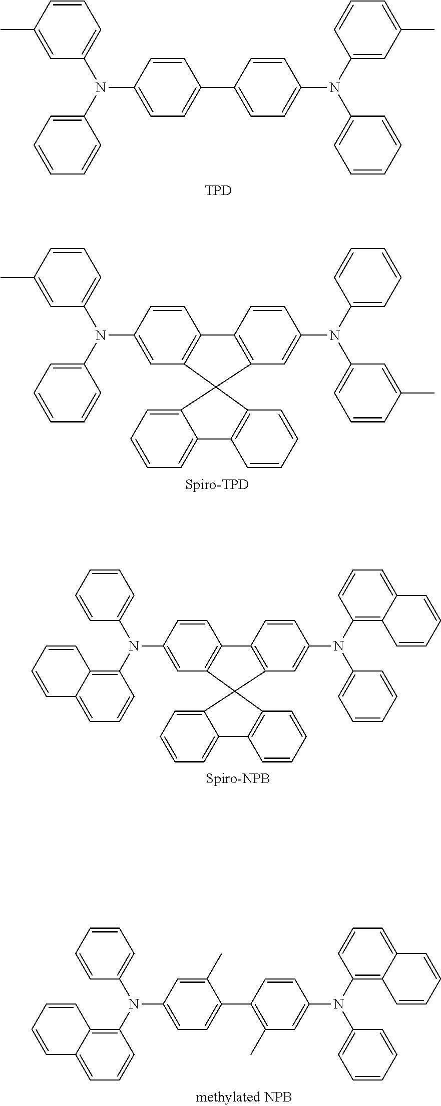

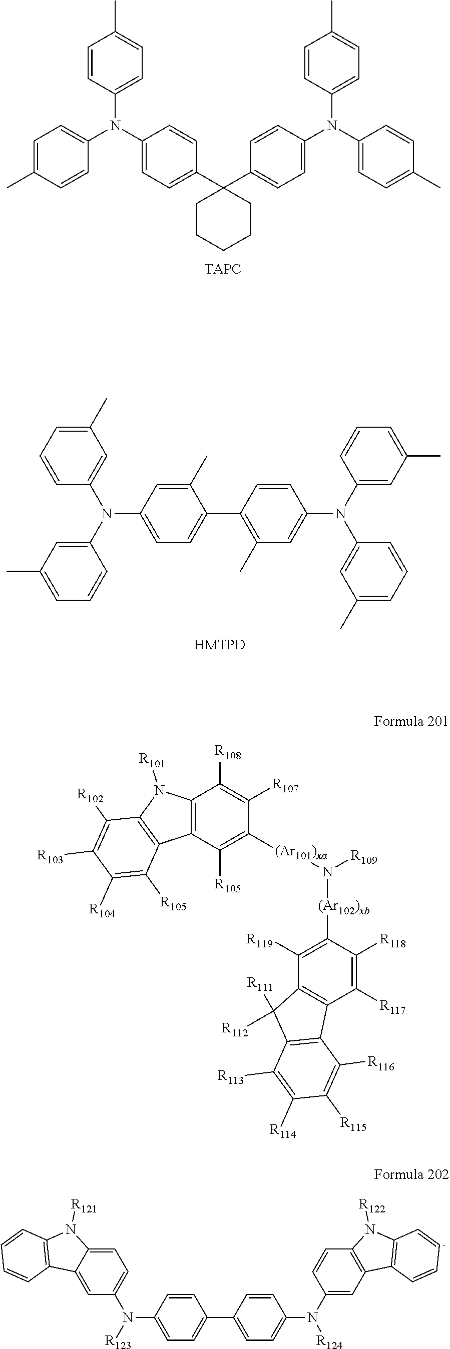

Ar₁₀₁ and Ar₁₀₂ in Formula 201 may each independently be selected from:

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group; and

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

In Formula 201, xa and xb may each independently be an integer selected from 0 to 5, or 0, 1, or 2. For example, xa is 1 and xb is 0, but xa and xb are not limited thereto.

R₁₀₁ to R₁₀₈, R₁₁₁ to R₁₁₉, and R₁₂₁ to R₁₂₄ in Formulae 201 and 202 may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, pentyl group, a hexyl group, etc.), and a C₁-C₁₀ alkoxy group (for example, a methoxy group, an ethoxy group, a propoxy group, a butoxy group, a pentoxy group, etc.);

a C₁-C₁₀ alkyl group and a C₁-C₁₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;

a phenyl group, a naphthyl group, an anthracenyl group, a fluorenyl group, and a pyrenyl group; and

a phenyl group, a naphthyl group, an anthracenyl group, a fluorenyl group, and a pyrenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group, but embodiments of the present disclosure are not limited thereto.

R₁₀₉ in Formula 201 may be selected from:

a phenyl group, a naphthyl group, an anthracenyl group, and a pyridinyl group; and

a phenyl group, a naphthyl group, an anthracenyl group, and a pyridinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, and a pyridinyl group.

According to an embodiment, the compound represented by Formula 201 may be represented by Formula 201A, but is not limited thereto:

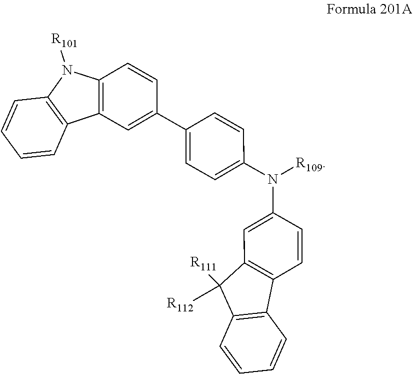

R₁₀₁, R₁₁₁, R₁₁₂, and R₁₀₉ in Formula 201A may be understood by referring to the description provided herein.

For example, the compound represented by Formula 201, and the compound represented by Formula 202 may include compounds HT1 to HT20 illustrated below, but embodiments of the present disclosure are not limited thereto:

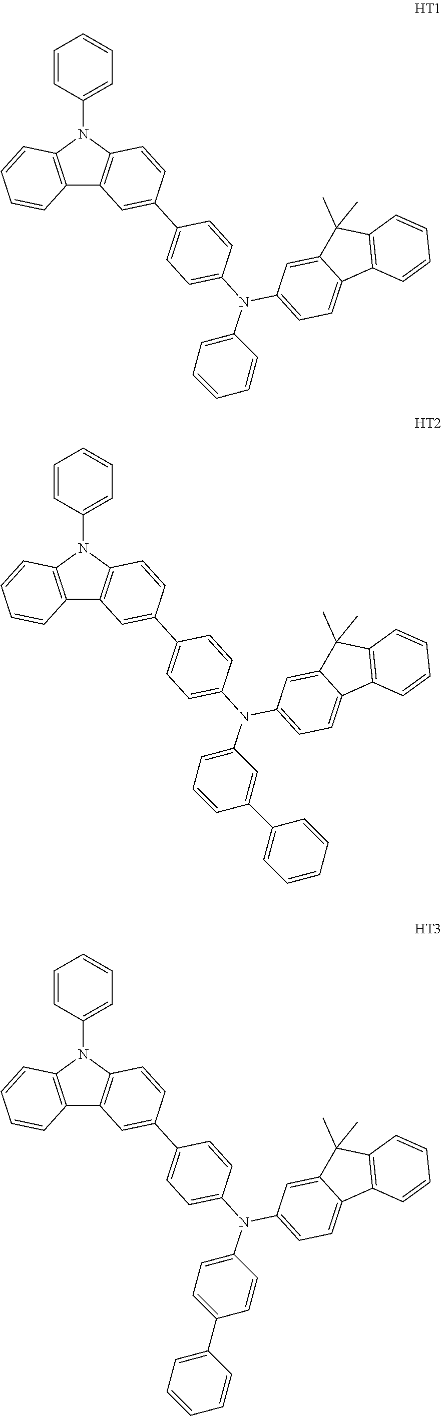 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

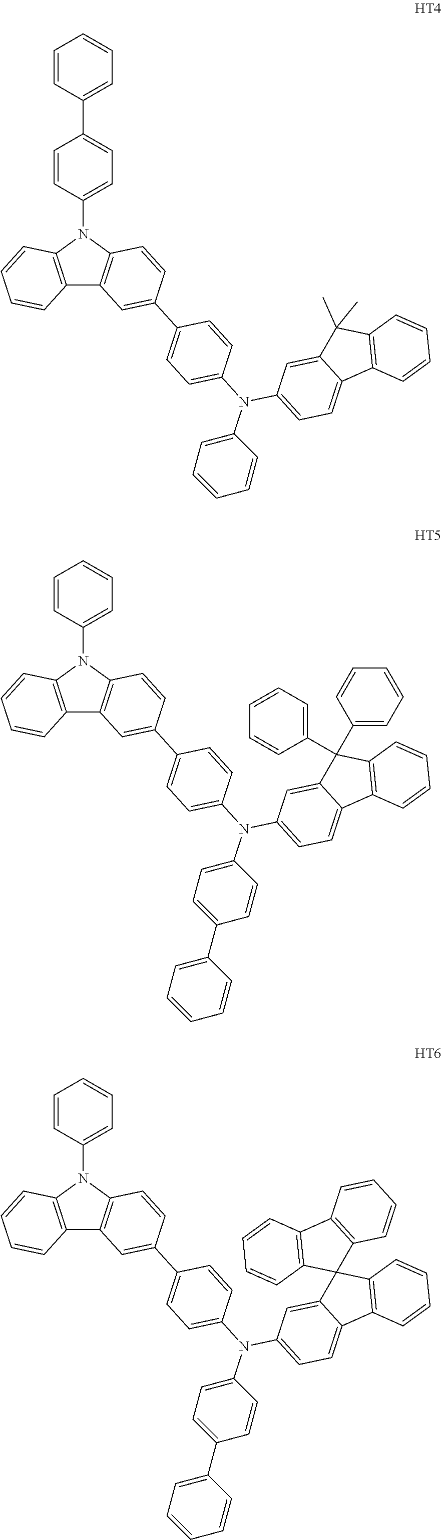 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

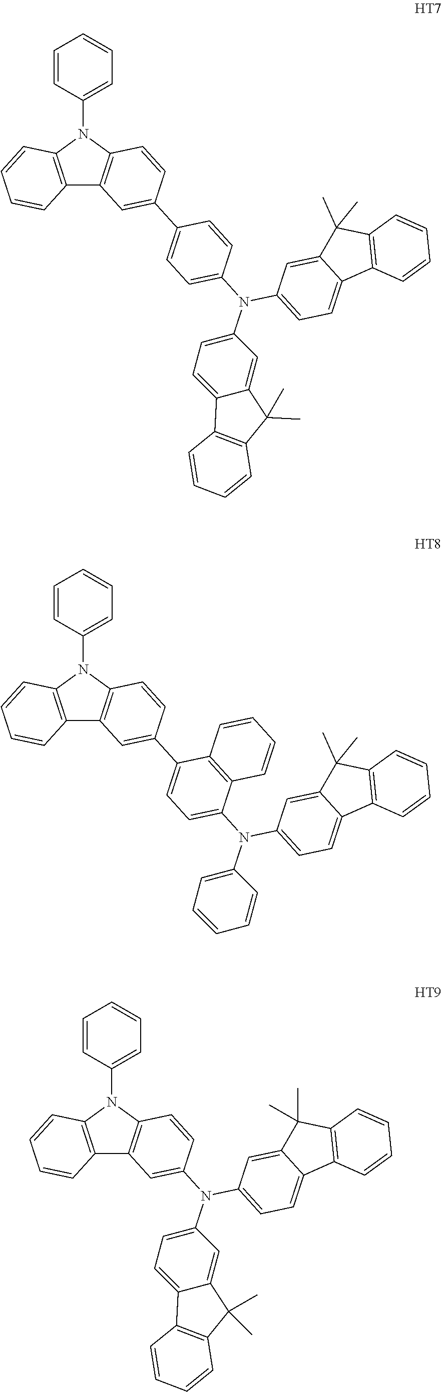 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21]

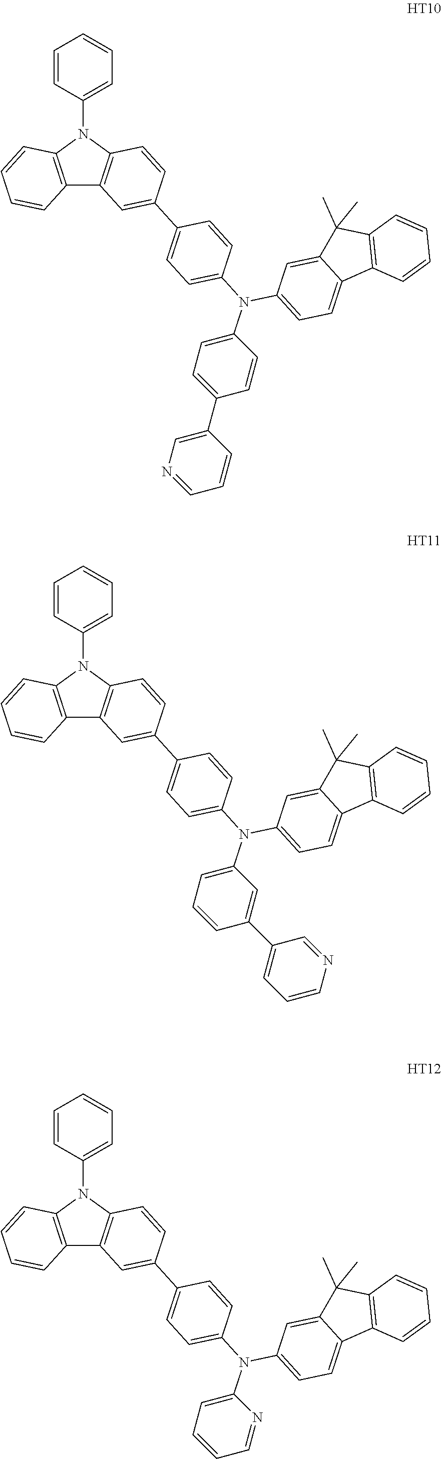 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

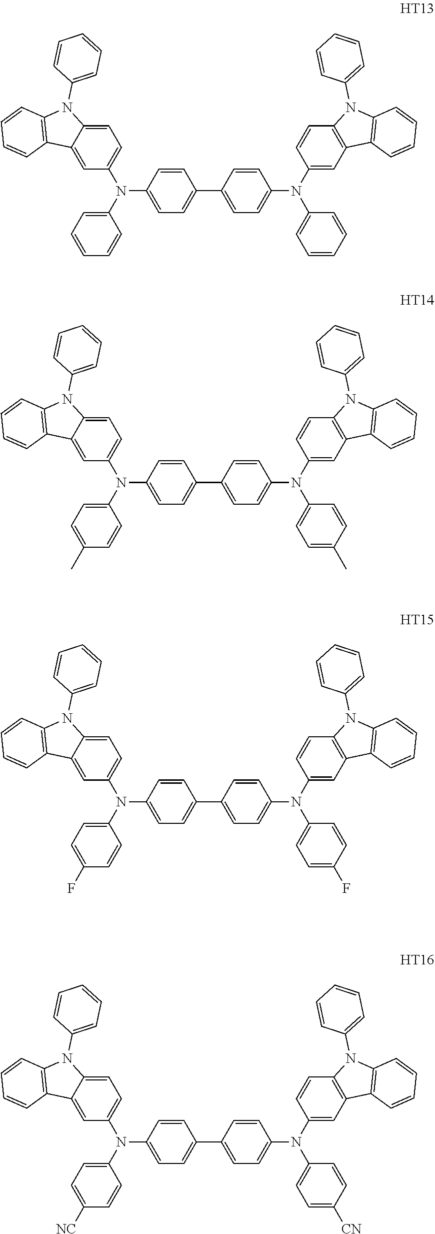 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1]

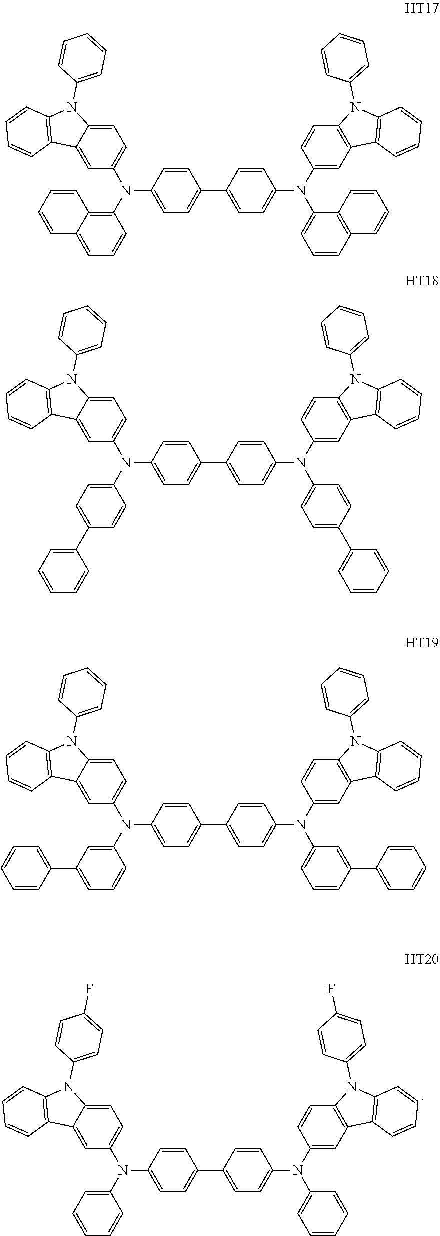 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in a range of about 100 Angstroms (Å) to about 10,000 Å, for example, about 100 Å to about 1,000 Å. When the hole transport region includes at least one of a hole injection layer and a hole transport layer, the thickness of the hole injection layer may be in a range of about 100 Å to about 10,000 Å, and for example, about 100 Å to about 1,000 Å, and the thickness of the hole transport layer may be in a range of about 50 Å to about 2,000 Å, and for example, about 100 Å to about 1,500 Å. While not wishing to be bound by theory, it is understood that when the thicknesses of the hole transport region, the hole injection layer and the hole transport layer are within these ranges, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be one selected from a quinone derivative, a metal oxide, and a cyano group-containing compound, but embodiments are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenium oxide; and a cyano group-containing compound, such as Compound HT-D1 below, but are not limited thereto.

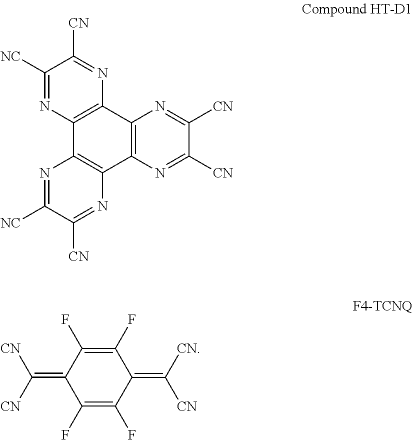

The hole transport region may include a buffer layer.

Also, the buffer layer may compensate for an optical resonance distance according to a wavelength of light emitted from the emission layer, and thus, efficiency of a formed organic light-emitting device may be improved.

Then, an emission layer (EML) may be formed on the hole transport region by vacuum deposition, spin coating, casting, LB deposition, or the like. When the emission layer is formed by vacuum deposition or spin coating, the deposition or coating conditions may be similar to those applied to form the hole injection layer although the deposition or coating conditions may vary according to the material that is used to form the emission layer.

Meanwhile, when the hole transport region includes an electron blocking layer, a material for the electron blocking layer may be selected from materials for the hole transport region described above and first through fourth compounds. However, the material for the electron blocking layer is not limited thereto. For example, when the hole transport region includes an electron blocking layer, a material for the electron blocking layer may be mCP, which will be explained later.

The emission layer may include a host and a dopant, the dopant may include the organometallic compound represented by Formula 1, and the host may include at least one selected from the first compound to the fourth compound.

When the organic light-emitting device is a full color organic light-emitting device, the emission layer may be patterned into a red emission layer, a green emission layer, and a blue emission layer. In one or more embodiments, due to a stack structure including a red emission layer, a green emission layer, and/or a blue emission layer, the emission layer may emit white light.

When the emission layer includes a host and a dopant, an amount of the dopant may be in a range of about 0.01 to about 15 parts by weight based on 100 parts by weight of the host, but is not limited thereto.

A thickness of the emission layer may be in a range of about 100 Å to about 1,000 Å, for example, about 200 Å to about 600 Å. While not wishing to be bound by theory, it is understood that when the thickness of the emission layer is within this range, excellent light-emission characteristics may be obtained without a substantial increase in driving voltage.

Then, an electron transport region may be disposed on the emission layer.

The electron transport region may include at least one selected from a hole blocking layer, an electron transport layer, and an electron injection layer.

For example, the electron transport region may have a hole blocking layer/electron transport layer/electron injection layer structure or an electron transport layer/electron injection layer structure, but the structure of the electron transport region is not limited thereto. The electron transport layer may have a single-layered structure or a multi-layered structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, at least one of BCP, Bphen, and BAlq but is not limited thereto.

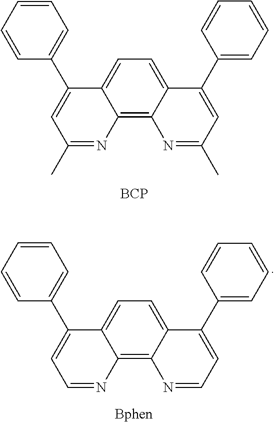

A thickness of the hole blocking layer may be in a range of about 20 Å to about 1,000 Å, for example, about 30 Å to about 300 Å. While not wishing to be bound by theory, it is understood that when the thickness of the hole blocking layer is within these ranges, the hole blocking layer may have improved hole blocking ability without a substantial increase in driving voltage.

The electron transport layer may further include at least one selected from BCP, Bphen, Alq₃, BAlq, TAZ, and NTAZ.

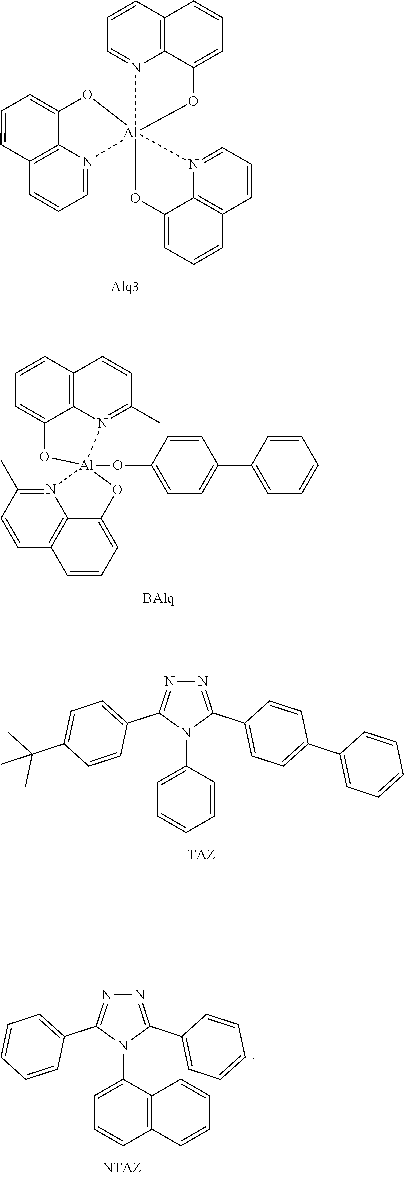

In one or more embodiments, the electron transport layer may include a compound represented by Formula 440 or Formula 441:

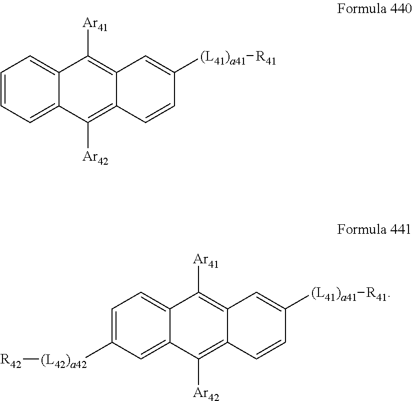

L₄₁ and L₄₂ in Formulae 440 and 441 may each independently be selected from:

a C₆-C₆₀ arylene group, a C₂-C₆₀ heteroarylene group, a divalent non-aromatic condensed polycyclic group, and a divalent non-aromatic condensed heteropolycyclic group; and

a C₆-C₆₀ arylene group, a C₂-C₆₀ heteroarylene group, a divalent non-aromatic condensed polycyclic group, and a divalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a pyrenyl group, a chrysenyl group, a phenanthrenyl group, a pyridinyl group, a pyrimidinyl group and a triazinyl group,

a41 and a42 in Formulae 440 and 441 may each independently be an integer selected from 0 to 5,

Ar₄₁ and Ar₄₂ in Formulae 440 and 441 may each independently be selected from:

a C₆-C₆₀ aryl group, a C₂-C₆₀ heteroaryl group, a monovalent non-aromatic condensed polycyclic group, and a monovalent non-aromatic condensed heteropolycyclic group; and

a C₆-C₆₀ aryl group, a C₂-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a pyrenyl group, a chrysenyl group, a phenanthrenyl group, a pyridinyl group, a pyrimidinyl group, and a triazinyl group,

R₄₁ and R₄₂ in Formulae 440 and 441 may each independently be selected from:

a benzimidazolyl group, a benzoxazolyl group, a benzothiazolyl group, benzopyrimidinyl group, an imidazopyridinyl group, a quinolinyl group, an isoquinolinyl group, a quinazolinyl group, a pyridinyl group, a pyrimidinyl group, a pyrazinyl group, a phenyl group, a naphthyl group, a pyrenyl group, a chrysenyl group, a fluorenyl group, and a phenanthrenyl group; and

a benzimidazolyl group, a benzoxazolyl group, a benzothiazolyl group, benzopyrimidinyl group, an imidazopyridinyl group, a quinolinyl group, an isoquinolinyl group, a quinazolinyl group, a pyridinyl group, a pyrimidinyl group, a pyrazinyl group, a phenyl group, a naphthyl group, a pyrenyl group, a chrysenyl group, a fluorenyl group, and a phenanthrenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a benzimidazolyl group, a benzoxazolyl group, a benzothiazolyl group, benzopyrimidinyl group, an imidazopyridinyl group, a quinolinyl group, an isoquinolinyl group, a quinazolinyl group, a pyridinyl group, a pyrimidinyl group, a pyrazinyl group, a phenyl group, a naphthyl group, a pyrenyl group, a chrysenyl group, a fluorenyl group, and a phenanthrenyl group.

L₄₁ and L₄₂ in Formulae 440 and 441 may be the same as described in connection with L₂.

In one or more embodiments, the electron transport layer may include a compound represented by Formula 442:

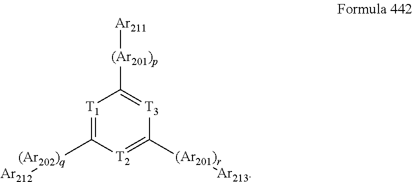

In Formula 42, T₁ may be N or C(R₂₀₁), T₂ may be N or C(R₂₀₂), T₃ may be N or C(R₂₀₃), and at least one selected from T₁ to T₃ may be N,

R₂₀₁ to R₂₀₃ in Formulae 440 and 441 may each independently selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₆-C₆₀ aryl group, a C₂-C₆₀ heteroaryl group, a monovalent non-aromatic condensed polycyclic group, and a monovalent non-aromatic condensed heteropolycyclic group; and

a C₆-C₆₀ aryl group, a C₂-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a pyrenyl group, a chrysenyl group, a phenanthrenyl group, a pyridinyl group, a pyrimidinyl group, and a triazinyl group,

Ar₂₀₁ to Ar₂₀₃ in Formulae 440 and 441 may each independently be selected from:

a C₆-C₆₀ arylene group, a C₂-C₆₀ heteroarylene group, a divalent non-aromatic condensed polycyclic group, and a divalent non-aromatic condensed heteropolycyclic group; and

a C₆-C₆₀ arylene group, a C₂-C₆₀ heteroarylene group, a divalent non-aromatic condensed polycyclic group, and a divalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a pyrenyl group, a chrysenyl group, a phenanthrenyl group, a pyridinyl group, a pyrimidinyl group, and a triazinyl group;

p, q, and r in Formulae 440 and 441 may each independently be 0, 1, or 2; and

Ar₂₁₁ and Ar₂₁₃ in Formulae 440 and 441 may each independently be selected from:

a C₆-C₆₀ aryl group, a C₂-C₆₀ heteroaryl group, a monovalent non-aromatic condensed polycyclic group, and a monovalent non-aromatic condensed heteropolycyclic group; and

a C₆-C₆₀ aryl group, a C₂-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a pyrenyl group, a chrysenyl group, a phenanthrenyl group, a pyridinyl group, a pyrimidinyl group, and a triazinyl group.

In an embodiment, at least two selected from T₁ to T₃ in Formula 442 may be N.

In one or more embodiments, T₁ to T3 in Formula 442 may all be N.

Ar₂₀₁ to Ar₂₀₃ in Formula 442 may each independently be selected from:

a phenylene group, a naphthylene group, anthrylene group, a pyrenylene group, a fluorenylene group, a triphenylenyl group, a pyridinylene group and a pyrimidinylene group; and

a phenylene group, a naphthylene group, anthrylene group, a pyrenylene group, a fluorenylene group, a triphenylenyl group, a pyridinylene group, and a pyrimidinylene group, each substituted with at least one selected from a phenyl group, a naphthyl group, an anthryl group, a pyrenyl group, a fluorenyl group, a triphenylenyl group, a pyridinyl group, and a pyrimidinyl group, but embodiments of the present disclosure are not limited thereto.

p, q, and r in Formula 442 may each independently be 0, 1, or 2. In an embodiment, p, q, and r in Formula 442 may each independently be 0 or 1, but embodiments of the present disclosure are not limited thereto.

Ar₂₁₁ to Ar₂₁₃ in Formula 442 may each independently be selected from:

a phenyl group, a naphthyl group, a pyrenyl group, a chrysenyl group, a fluorenyl group, a phenanthrenyl group, a benzimidazolyl group, a benzoxazolyl group, a benzothiazolyl group, benzopyrimidinyl group, an imidazopyrimidinyl group, a quinolinyl group, an isoquinolinyl group, a quinazolinyl group, a pyridinyl group, a pyrimidinyl group, a pyrazinyl group, and a triazinyl group; and

a phenyl group, a naphthyl group, a pyrenyl group, a chrysenyl group, a fluorenyl group, a phenanthrenyl group, a benzimidazolyl group, a benzoxazolyl group, a benzothiazolyl group, benzopyrimidinyl group, an imidazopyrimidinyl group, a quinolinyl group, an isoquinolinyl group, a quinazolinyl group, a pyridinyl group, a pyrimidinyl group, a pyrazinyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a pyrenyl group, a chrysenyl group, a phenanthrenyl group, a pyridinyl group, a pyrimidinyl group, and a triazinyl group, but embodiments of the present disclosure are not limited thereto.

In an embodiment, at least one selected from Ar₂₁₁ to Ar₂₁₃ in Formula 442 may each independently be selected from:

a benzimidazolyl group, a benzoxazolyl group, a benzothiazolyl group, benzopyrimidinyl group, an imidazopyrimidinyl group, a quinolinyl group, an isoquinolinyl group, a quinazolinyl group, a pyridinyl group, a pyrimidinyl group, a pyrazinyl group, and a triazinyl group; and

a benzimidazolyl group, a benzoxazolyl group, a benzothiazolyl group, benzopyrimidinyl group, an imidazopyrimidinyl group, a quinolinyl group, an isoquinolinyl group, a quinazolinyl group, a pyridinyl group, a pyrimidinyl group, a pyrazinyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a pyrenyl group, a chrysenyl group, a phenanthrenyl group, a pyridinyl group, a pyrimidinyl group, and a triazinyl group, but embodiments of the present disclosure are not limited thereto.

In one or more embodiments, at least one selected from Ar₂₁₁ to Ar₂₁₃ in Formula 442 may be a substituted or unsubstituted phenanthrenyl group.

For example, the electron transport region may include at least one selected from Compounds ET1 to ET16, but embodiments of the present disclosure are not limited thereto:

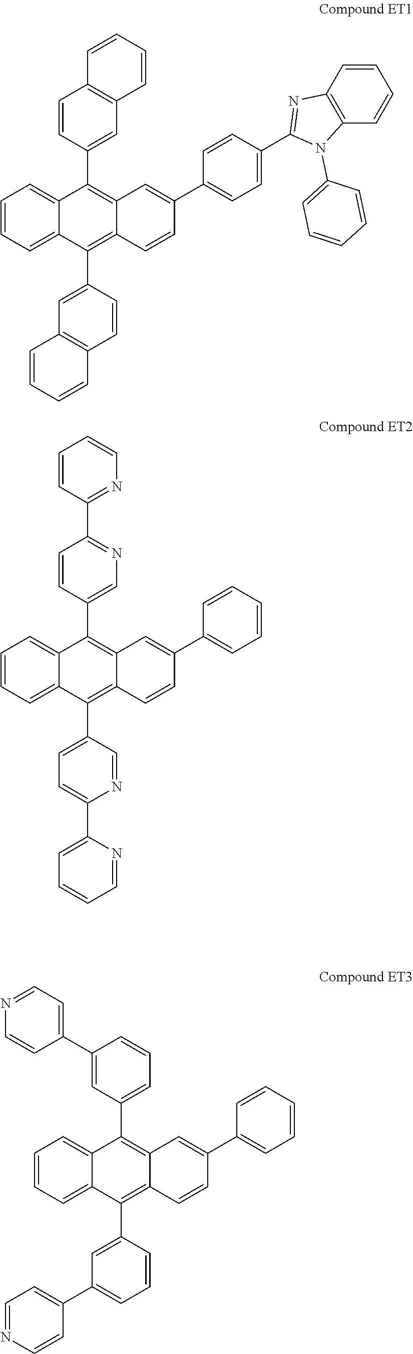

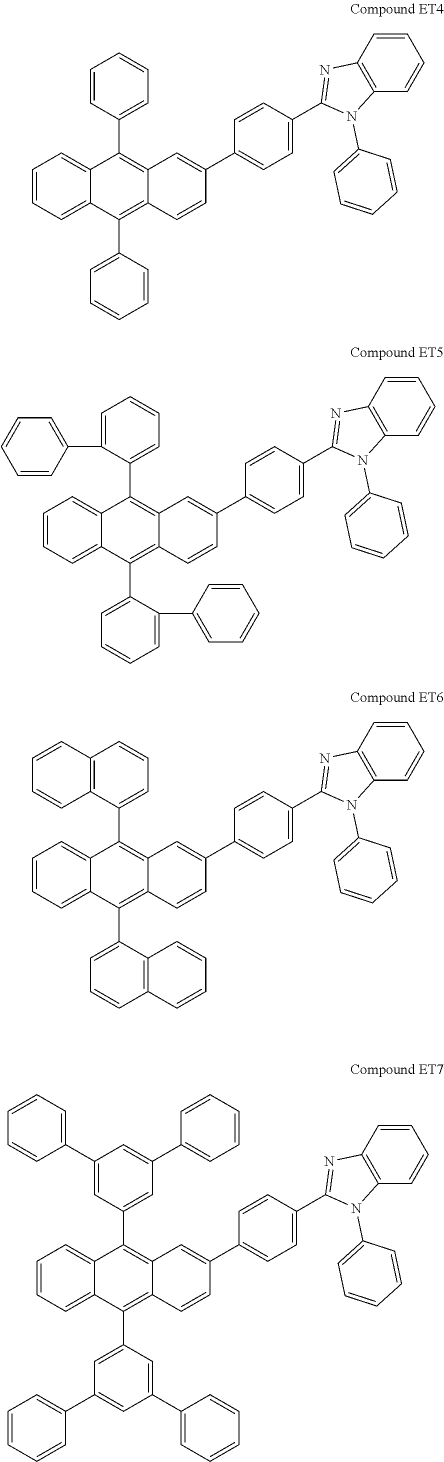 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

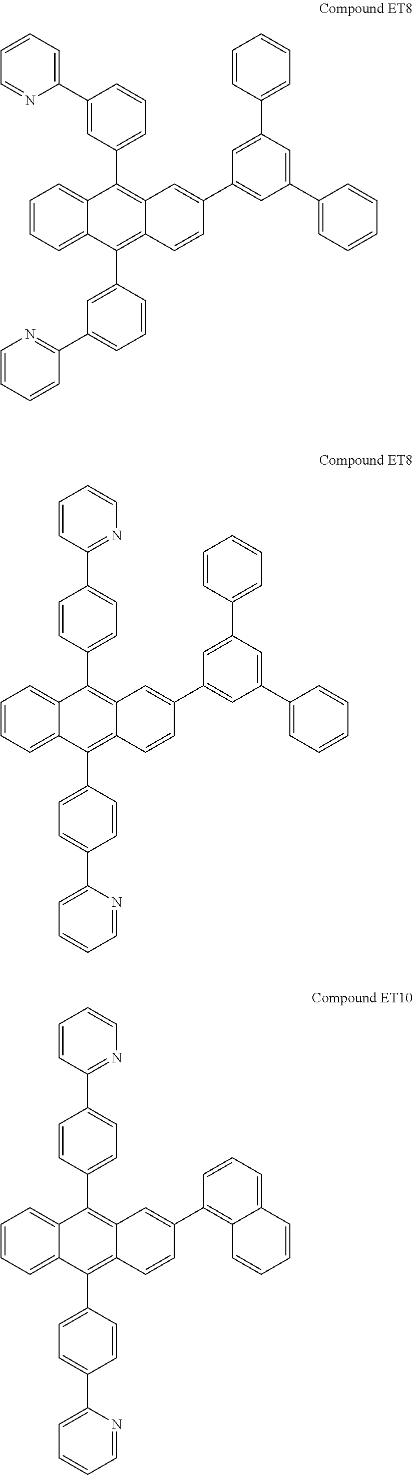 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

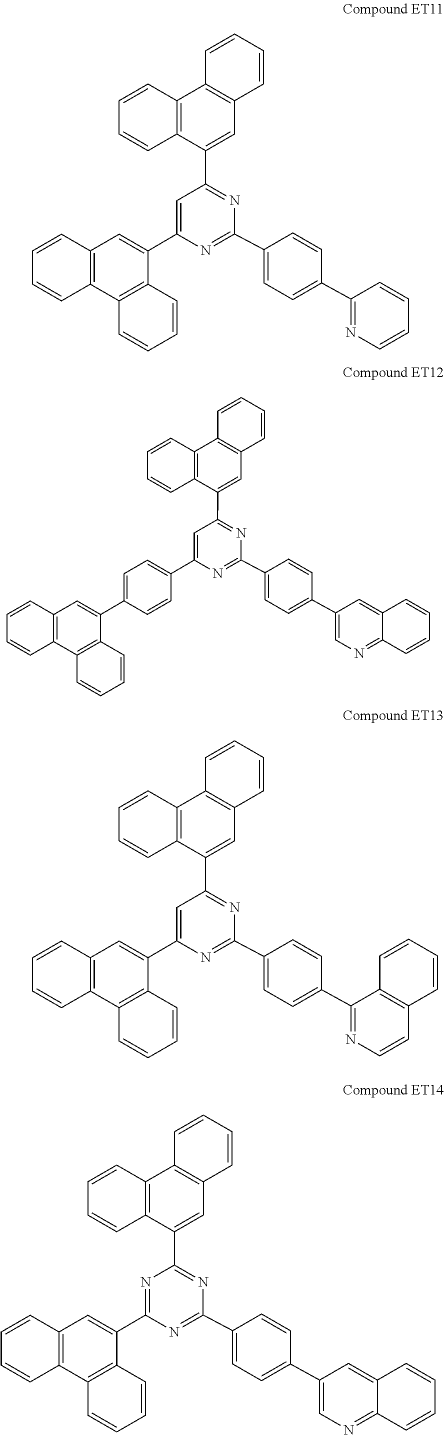 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

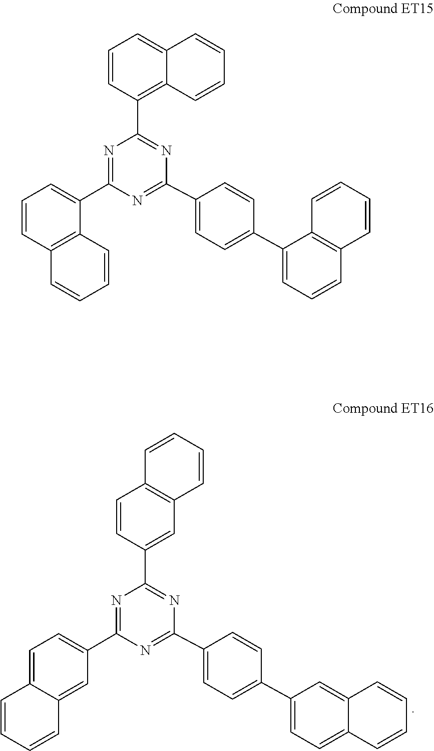 [SMILES: c1ccc2c(-c3ccc(-c4nc(-c5cccc6ccccc56)nc(-c5cccc6ccccc56)n4)cc3)cccc2c1 ; c1ccc2cc(-c3ccc(-c4nc(-c5ccc6ccccc6c5)nc(-c5ccc6ccccc6c5)n4)cc3)ccc2c1]

In an embodiment, the electron transport region may include an electron transport layer, and the electron transport layer may include a compound represented by at least one selected from Formulae 440, 441, and 442, but embodiments of the present disclosure are not limited thereto.

A thickness of the electron transport layer may be in a range of about 100 Å to about 1,000 Å, for example, about 150 Å to about 500 Å. While not wishing to be bound by theory, it is understood that when the thickness of the electron transport layer is within the range described above, the electron transport layer may have satisfactory electron transport characteristics without a substantial increase in driving voltage.

Also, the electron transport layer may further include, in addition to the materials described above, a metal-containing material.

The metal-containing material may include a Li complex. The Li complex may include, for example, Compound ET-D1 (lithium quinolate, LiQ) or ET-D2.

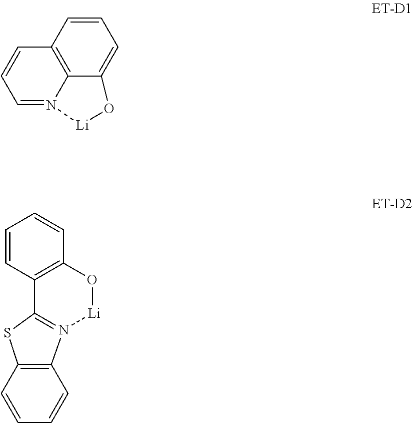

The electron transport region may include an electron injection layer (EIL) that promotes flow of electrons from the second electrode 19 thereinto.

The electron injection layer may include at least one selected from, LiF, NaCl, CsF, Li₂O, BaO, and LiQ.

A thickness of the electron injection layer may be in a range of about 1 Å to about 100 Å, for example, about 3 Å to about 90 Å. While not wishing to be bound by theory, it is understood that when the thickness of the electron injection layer is within the range described above, the electron injection layer may have satisfactory electron injection characteristics without a substantial increase in driving voltage.

The second electrode 19 is disposed on the organic layer 15. The second electrode 19 may be a cathode. A material for forming the second electrode 19 may be selected from metal, an alloy, an electrically conductive compound, and a combination thereof, which have a relatively low work function. For example, lithium (Li), magnesium (Mg), aluminum (Al), aluminum-lithium (Al—Li), calcium (Ca), magnesium-indium (Mg—In), or magnesium-silver (Mg—Ag) may be used as a material for forming the second electrode 19. In one or more embodiments, to manufacture a top emission type light-emitting device, a transmissive electrode formed using ITO or IZO may be used as the second electrode 19.

Hereinbefore, the organic light-emitting device has been described with reference to FIG. 1, but is not limited thereto.

The term “C₁-C₆₀ alkyl group” as used herein refers to a linear or branched saturated aliphatic hydrocarbon monovalent group having 1 to 60 carbon atoms. Non-limiting examples thereof include a methyl group, an ethyl group, a propyl group, an iso-butyl group, a sec-butyl group, a tert-butyl group, a pentyl group, an iso-amyl group, and a hexyl group. The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by —OA₁₀₁ (wherein A₁₀₁ is the C₁-C₆₀ alkyl group). Non-limiting examples thereof include a methoxy group, an ethoxy group, and an iso-propyloxy (iso-propoxy) group.

The term “C₂-C₆₀ alkenyl group” as used herein refers to a hydrocarbon group formed by substituting at least one carbon-carbon double bond in the middle or at the terminus of the C₂-C₆₀ alkyl group. Examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by substituting at least one carbon-carbon triple bond in the middle or at the terminus of the C₂-C₆₀ alkyl group. Examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocyclic group having 3 to 10 carbon atoms. Non-limiting examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent saturated monocyclic group having at least one heteroatom selected from N, O, P, Si and S as a ring-forming atom and 1 to 10 carbon atoms. Non-limiting examples thereof include a tetrahydrofuranyl group and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has 3 to 10 carbon atoms and at least one carbon-carbon double bond in the ring thereof, and which is not aromatic. Non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent monocyclic group that has at least one heteroatom selected from N, O, P, Si, and S as a ring-forming atom, 1 to 10 carbon atoms, and at least one carbon-carbon double bond in its ring. Examples of the C₁-C₁₀ heterocycloalkenyl group are a 2,3-dihydrofuranyl group and a 2,3-dihydrothiophenyl group. The term “C₁-C₁₀ heterocycloalkenylene group” as used herein refers to a divalent group having the same structure as the C₁-C₁₀ heterocycloalkenyl group.

A C₆-C₆₀ aryl group as used herein refers to a monovalent group having a carbocyclic aromatic system having 6 to 60 carbon atoms, and a C₆-C₆₀ arylene group as used herein refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₁-C₆₀ heteroaryl group” as used herein refers to a monovalent group having a heterocyclic aromatic system that has at least one heteroatom selected from N, O, P, Si, and S as a ring-forming atom, and 1 to 60 carbon atoms. The term “C₁-C₆₀ heteroarylene group,” as used herein refers to a divalent group having a heterocyclic aromatic system that has at least one heteroatom selected from N, O, P, Si, and S as a ring-forming atom, and 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, the rings may be fused to each other.

The term “C₆-C₆₀ aryloxy group” as used herein indicates —OA₁₀₂ (wherein A₁₀₂ is the C₆-C₆₀ aryl group), and a C₆-C₆₀ arylthio group as used herein indicates —SA₁₀₃ (wherein A₁₀₃ is the C₆-C₆₀ aryl group).

The term “monovalent non-aromatic condensed polycyclic group” as used herein refers to a monovalent group (for example, having 8 to 60 carbon atoms) that has two or more rings condensed to each other, only carbon atoms as a ring-forming atom, and which is non-aromatic in the entire molecular structure. Examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group (for example, having 2 to 60 carbon atoms) that has two or more rings condensed to each other, has a heteroatom selected from N, O, P, and S, other than carbon atoms, as a ring-forming atom, and which is non-aromatic in the entire molecular structure. Non-limiting examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group.

In the present specification, at least one substituent selected from a substituent(s) of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and

—N(Q₃₁)(Q₃₂), —Si(Q₃₃)(Q₃₄)(Q₃₅), —B(Q₃₆)(Q₃₇) and —P(═O)(Q₃₈)(Q₃₉),

wherein Q₁ to Q₉, Q₁₁ to Q₁₉, to Q₂₉, and Q₃₁ to Q₃₉ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

When a group containing a specified number of carbon atoms is substituted with any of the groups listed in the preceding paragraphs, the number of carbon atoms in the resulting “substituted” group is defined as the sum of the carbon atoms contained in the original (unsubstituted) group and the carbon atoms (if any) contained in the substituent. For example, when the term “substituted C₁-C₆₀ alkyl” refers to a C₁-C₆₀ alkyl group substituted with C₆-C₆₀ aryl group, the total number of carbon atoms in the resulting aryl substituted alkyl group is C₇-C₁₂₀.

Hereinafter, a compound and an organic light-emitting device according to embodiments are described in detail with reference to Synthesis Example and Examples. However, the organic light-emitting device is not limited thereto. The wording “B was used instead of A” used in describing Synthesis Examples means that an amount of A used was identical to an amount of B used, in terms of a molar equivalent.

### EXAMPLES

### Synthesis Example 1: Synthesis of Compound 1

Synthesis of Compound A1

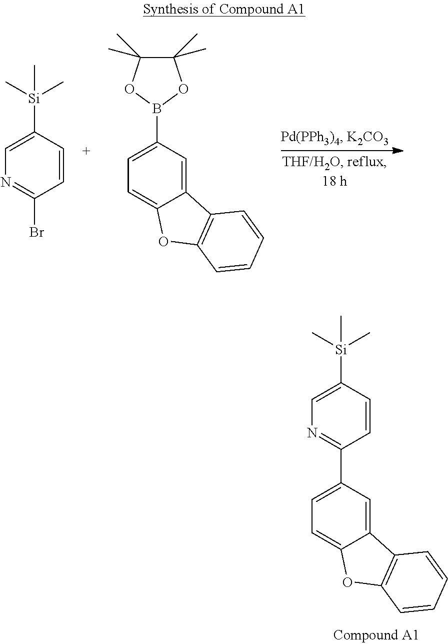

9 grams (g) (39.09 millimoles, mmol) of 2-bromo-5-(trimethylsilyl)pyridine, 13.22 g (44.96 mmol) of 2-(dibenzo[b,d]furan-2-yl)-4,4,5,5-tetramethyl-1,3,2-dioxaborolane, 2.26 g (1.95 mmol) of Pd(PPh₃)₄, and 16.21 g (117.27 mmol) of K₂CO₃ were mixed with 210 milliliters (mL) of tetrahydrofuran (THF) and 70 mL of distilled water. The mixture was agitated under reflux for 18 hours. The temperature was reduced to room temperature, the organic layer was extracted by using methylene chloride (MC), anhydrous magnesium sulfate (MgSO₄) was added thereto to perform dehydration, and the dried solution was filtered. The obtained filtrate was concentrated under reduced pressure and the obtained residual was purified by column chromatography (MC:Hexane=1:1) to obtain 7.4 g (60%) of Compound A1.

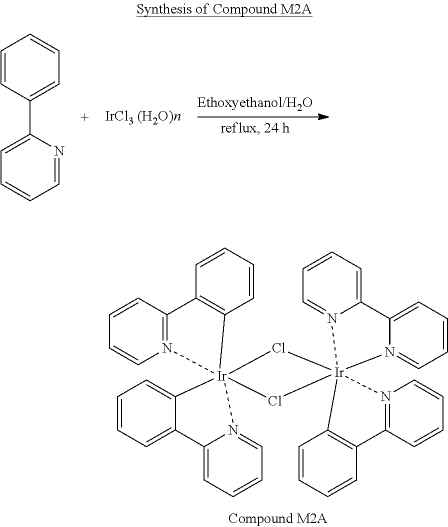

14.66 g (94.44 mmol) of 2-phenylpyridine and 14.80 g (41.97 mmol) of iridium chloride were mixed with 210 mL of ethoxyethanol and 70 mL of distilled water, and the mixture was agitated under reflux for 24 hours to carry out the reaction. Then, the temperature was reduced to room temperature. The generated solid was filtered, separated, and thoroughly washed with water, methanol, and hexane in that order. The obtained solid was dried in a vacuum oven to obtain 19.5 g (87%) of Compound M2A.

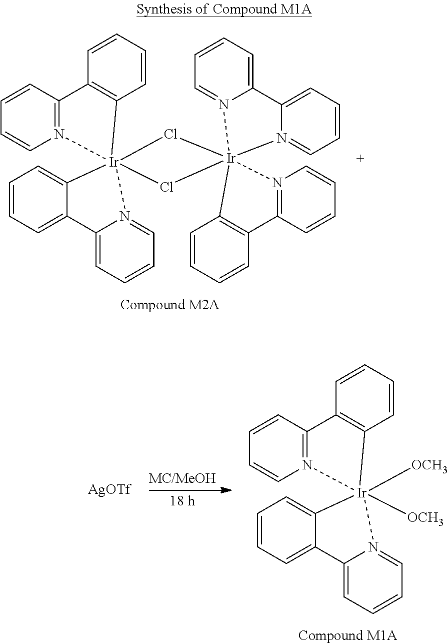

6.01 g (5.60 mmol) of Compound M2A was mixed with 45 mL of MC, 2.88 g (11.21 mmol) of AgOTf was dissolved in 15 mL of methanol and added thereto. The mixture was agitated for 18 hours at room temperature while blocking light with an aluminum foil to carry out the reaction. A solid (Compound M1A) obtained by removing a solid generated by celite filtration and decompressing a filtrate was used in the next reaction, without performing an additional purification thereon.

Synthesis of Compound 1

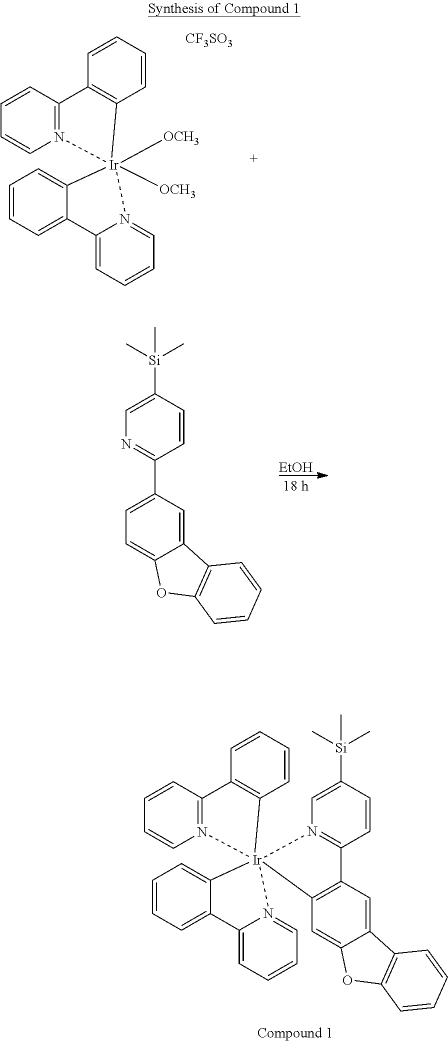

8 g (11.22 mmol) of Compound M1A and 4.28 g (13.47 mmol) of Compound A1 were mixed with 100 mL of ethanol, and the resultant was agitated under reflux for 18 hours to carry out the reaction. Then, the temperature was reduced to room temperature. The resulting mixture was filtered to separate a solid, which was then thoroughly washed with ethanol and hexane. Column chromatography was then performed thereon using MC and hexane at a ratio of 40:60 to obtain 2.54 g (28%) of Compound 1. The obtained product was confirmed by Mass and HPLC analysis.

HRMS (MALDI) calcd for C₄₂H₃₄IrN₃OSi: m/z 817.2100, Found: 817.2104

### Synthesis Example 2: Synthesis of Compound 3

Synthesis of Compound B1

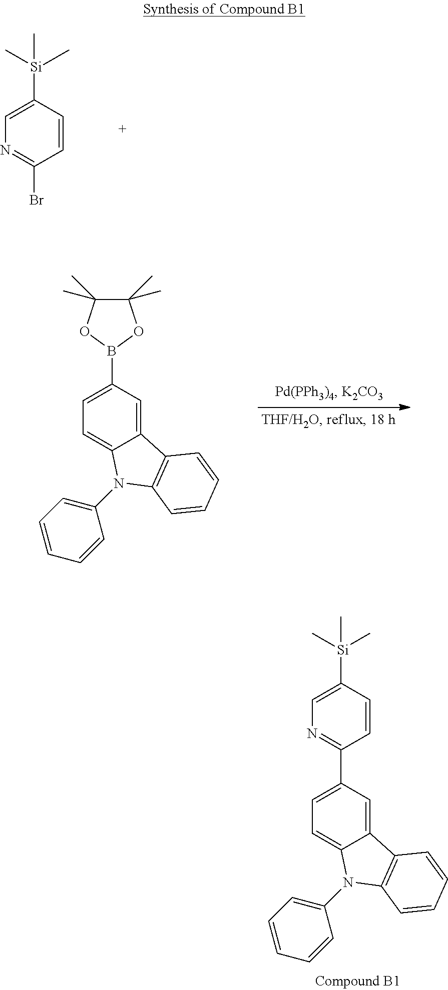 [SMILES: C[Si](C)(C)c1ccc(Br)nc1 ; CC1(C)OB(c2ccc3c(c2)c2ccccc2n3-c2ccccc2)OC1(C)C ; C[Si](C)(C)c1ccc(-c2ccc3c(c2)c2ccccc2n3-c2ccccc2)nc1]

8.5 g (55%) of Compound B1 was obtained in the same manner as Compound A1 in Synthesis Example 1, except that 13.47 g (46.92 mmol) of 9-phenyl-3-(4,4,5,5-tetramethyl-1,3,2-dioxaborolan-2-yl)-9H-carbazole were used instead of 13.22 g (44.96 mmol) of 2-(dibenzo[b,d]furan-2-yl)-4,4,5,5-tetramethyl-1,3,2-dioxaborolane.

Synthesis of Compound 3

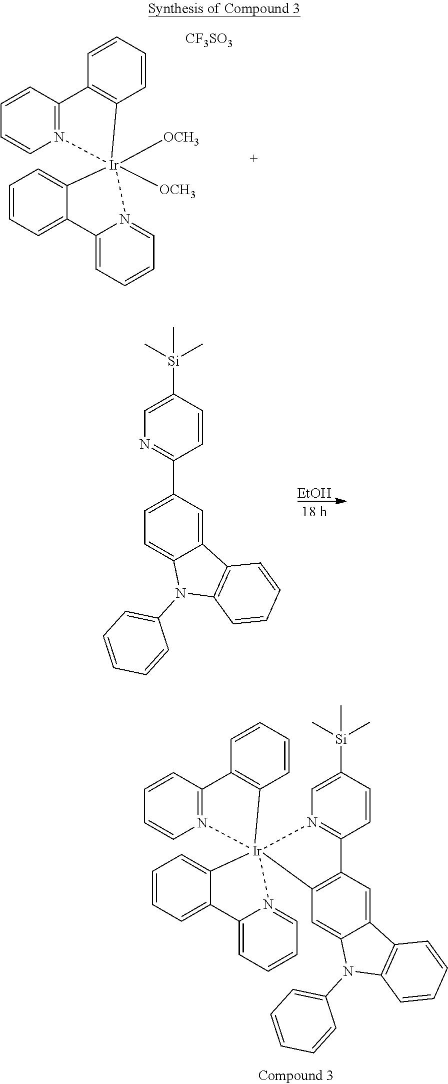

2.4 g (32%) of Compound 3 was obtained in the same manner as Compound 1 in Synthesis Example 1, except that 6.00 g (8.42 mmol) of Compound M1A and 3.97 g (10.10 mmol) of Compound B1 were respectively used instead of 8 g (11.22 mmol) of Compound M1A and 4.28 g (13.47 mmol) of Compound A1. The obtained product was confirmed by Mass and HPLC analysis.

HRMS (MALDI) calcd for C₄₈H₃₉IrN₄Si: m/z 892.2573, Found: 892.2575

### Synthesis Example 3: Synthesis of Compound 8

Synthesis of Compound C3

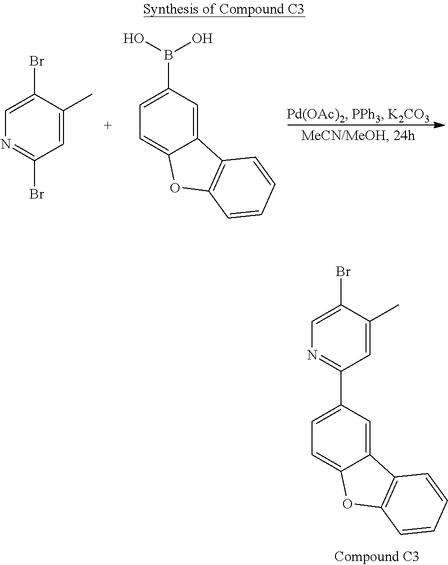

18.55 g (73.92 mmol) of 2,5-dibromo-4-methylpyridine, 18.81 g (88.70 mmol) of dibenzo[b,d]furan-2-ylboronic acid, 1.66 g (7.39 mmol) of Pd(OAc)₂, 3.88 g (14.78 mmol) of PPh₃, and 20.43 g (147.84 mmol) of K₂CO₃ were mixed with 200 mL of acetonitrile and 100 mL of methanol. The mixture was agitated at a temperature of 50° C. for 18 hours, and the resultant was cooled to room temperature and filtered. The organic layer was extracted therefrom by using MC, anhydrous magnesium sulfate (MgSO₄) was added thereto to perform dehydration, and the dried resulting solution was filtered. A filtrate was concentrated under reduced pressure and the obtained residual was purified by column chromatography (MC:Hx=60:40) to obtain 13.0 g (52%) of Compound C3.

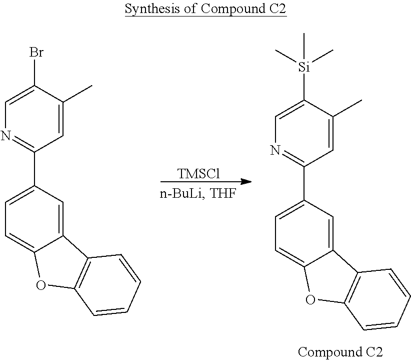 [SMILES: "TMSCl" ; Cc1cc(-c2ccc3oc4ccccc4c3c2)ncc1Br ; Cc1cc(-c2ccc3oc4ccccc4c3c2)ncc1[Si](C)(C)C]

12.24 g (36.20 mmol) of Compound C3 was mixed with THF 300 mL, and the mixture was cooled to −78° C. 33.94 mL (54.30 mmol) of 1.6 molar (M) n-BuLi was slowly added thereto and was agitated at −78° C. for 1 hour. Then, 6.89 mL (54.30 mmol) of TMSCl was added thereto, and a reaction was carried out at −78° C. for 1 hour. The temperature was then raised to room temperature and the reaction was further carried out for 12 hours. The organic layer obtained therefrom was extracted by using MC, and anhydrous magnesium sulfate was added thereto to remove water therefrom. The obtained filtrate was concentrated under reduced pressure and column chromatography was performed on the obtained residual with EA:Hexane=4:96 to obtain 8.0 g (67%) of Compound C2.

Synthesis of Compound 8

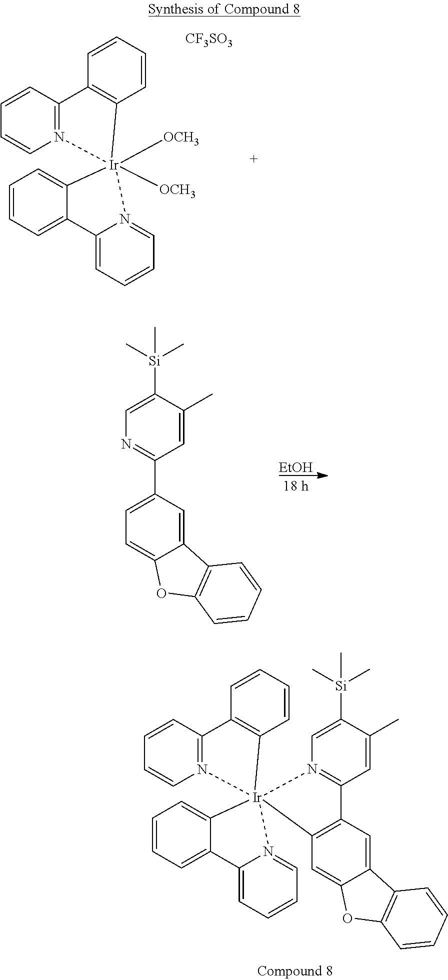

2.05 g (29%) of Compound 8 was obtained in the same manner as Compound 1 in Synthesis Example 1, except that 6.00 g (8.42 mmol) of Compound M1A and 3.35 g (10.11 mmol) of Compound C2 were respectively used instead of 8 g (11.22 mmol) of Compound M1A and 4.46 g (13.47 mmol) of Compound A1. The obtained product was confirmed by Mass and HPLC analysis.

HRMS (MALDI) calcd for C₄₃H₃₆IrN₃OSi: m/z 831.2257, Found: 831.2259

### Synthesis Example 4: Synthesis of Compound 22

Synthesis of Compound C1

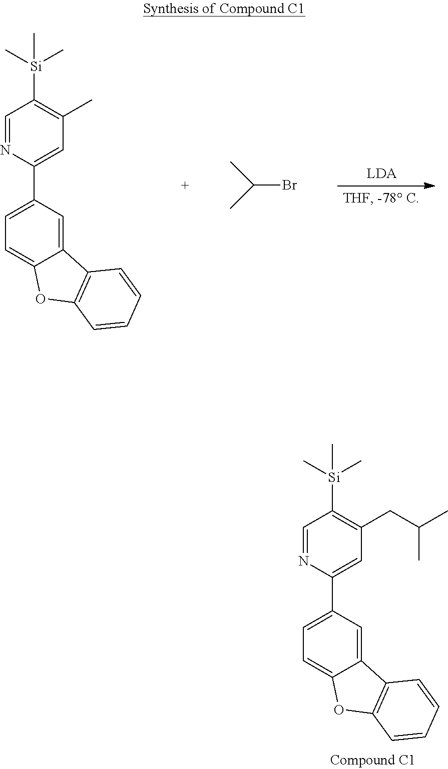

7.1 g (21.42 mmol) of Compound C2 was mixed with 100 mL of THF The mixture was cooled to −78° C. and 26.8 mL (53.54 mmol) of lithium diisopropylamide (LDA) was slowly added thereto. The resulting mixture was agitated at −78° C. for 1 hour to perform a reaction. The temperature was raised to room temperature and the reaction was further carried out for 1.5 hours. The temperature was then reduced to −78° C., 5.03 mL (53.54 mmol) of 2-bromopropane was slowly added to the resultant, the temperature was raised to room temperature, and the reaction was carried out for 12 hours. The organic layer obtained therefrom was extracted by using MC, and anhydrous magnesium sulfate was added thereto to remove water. The obtained filtrate was concentrated under reduced pressure and column chromatography was performed on the obtained residual with EA:Hexane=4:96 to obtain 6.80 g (85%) of Compound C1.

Synthesis of Compound 22

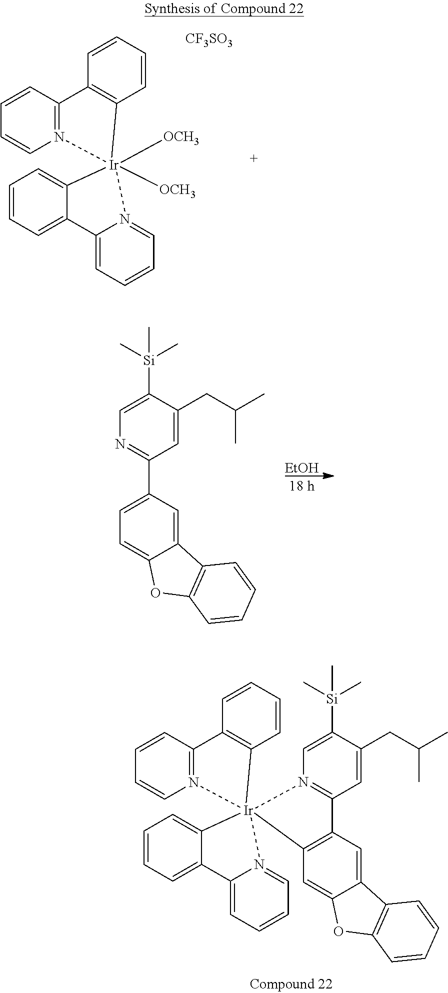

2.30 g (31%) of Compound 22 was obtained in the same manner as Compound 1 in Synthesis Example 1, except that 6.00 g (8.42 mmol) of Compound M1A and 3.77 g (10.10 mmol) of Compound C1 were respectively used instead of 8 g (11.22 mmol) of Compound M1A and 4.28 g (13.47 mmol) of Compound A1. The obtained product was confirmed by Mass and HPLC analysis.

HRMS (MALDI) calcd for C₄₆H₄₂IrN₃OSi: m/z 873.2726, Found: 873.2720

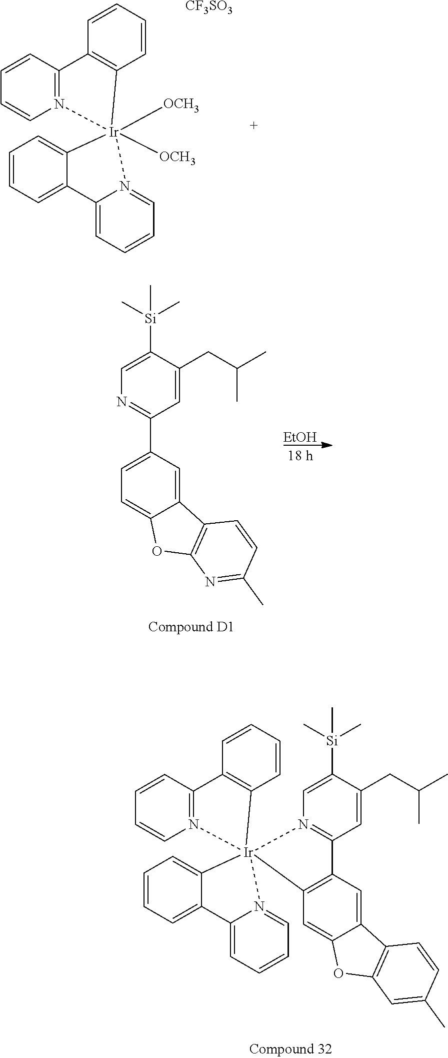

1.3 g (17%) of Compound 32 was obtained in the same manner as Compound 1 in Synthesis Example 1, except that 6.00 g (8.41 mmol) of Compound M1A and 3.92 g (10.09 mmol) of Compound D1 were respectively used instead of 8 g (11.22 mmol) of Compound M1A and 4.46 g (13.47 mmol) of Compound A1. The obtained product was confirmed by Mass and HPLC analysis.

HRMS (MALDI) calcd for C₄₆H₄₃IrN₄OSi: m/z 888.2835, Found: 888.2830

### Synthesis Example 6: Compound 35

Synthesis of Compound E3

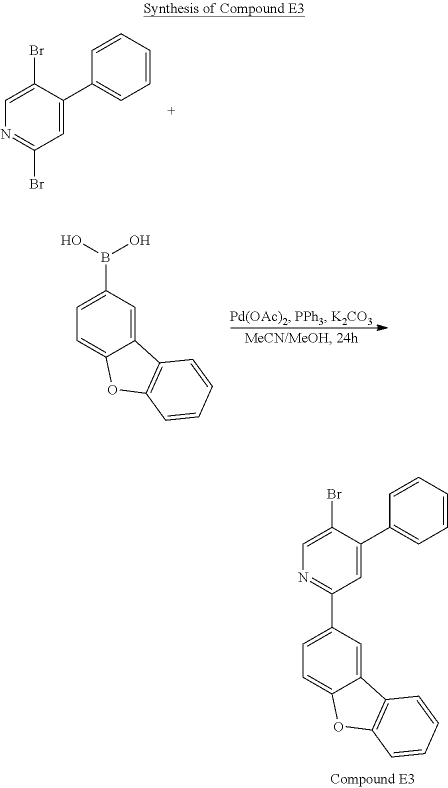

13.7 g (69%) of Compound E3 was obtained in the same manner as Compound C₃ in Synthesis Example 3, except that 15.64 g (49.97 mmol) of 2,5-dibromo-4-phenylpyridine and 12.71 g (55.96 mmol) of dibenzo[b,d]furan-2-ylboronic acid were respectively used instead of 18.55 g (73.92 mmol) of 2,5-dibromo-4-methylpyridine and 18.81 g (88.70 mmol) of dibenzo[b,d]furan-2-ylboronic acid.

Synthesis of Compound E2

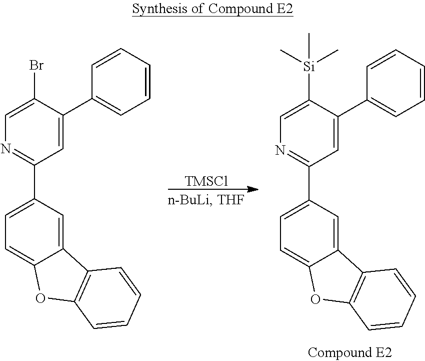 [SMILES: "TMSCl" ; Brc1cnc(-c2ccc3oc4ccccc4c3c2)cc1-c1ccccc1 ; C[Si](C)(C)c1cnc(-c2ccc3oc4ccccc4c3c2)cc1-c1ccccc1]

6.6 g (66%) of Compound E2 was obtained in the same manner as Compound C₂ in Synthesis Example 3, except that 10.17 g (25.41 mmol) of Compound E3 was used instead of 12.24 g (36.20 mmol) of Compound C3.

Synthesis of Compound 35

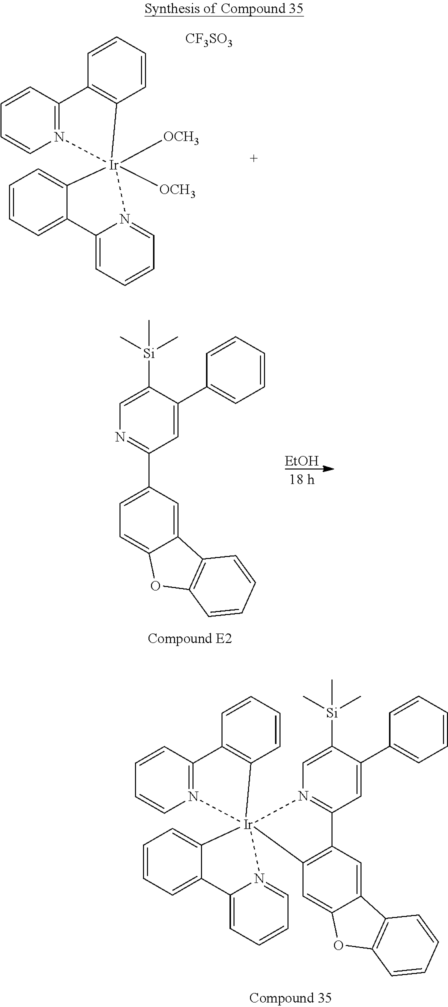

1.8 g (24%) of Compound 35 was obtained in the same manner as Compound 1 in Synthesis Example 1, except that 6.00 g (8.41 mmol) of Compound M1A and 3.97 g (10.09 mmol) of Compound E2 were respectively used instead of 8 g (11.22 mmol) of Compound M1A and 4.46 g (13.47 mmol) of Compound A1. The obtained product was confirmed by Mass and HPLC analysis.

HRMS (MALDI) calcd for C₄₈H₃₈IrN₃OSi: m/z 893.2413, Found: 893.2417

### Synthesis Example 7: Synthesis of Compound 146

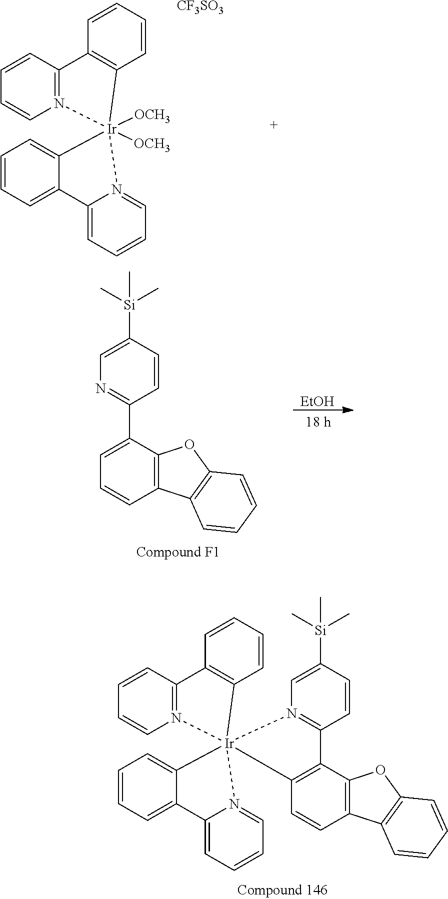

2.1 g (31%) of Compound 146 was obtained in the same manner as Compound 1 in Synthesis Example 1, except that 6.00 g (8.41 mmol) of Compound M1A and 3.2 g (10.09 mmol) of Compound F1 were respectively used instead of 8 g (11.22 mmol) of Compound M1A and 4.46 g (13.47 mmol) of Compound A1. The obtained product was confirmed by Mass and HPLC analysis.

HRMS (MALDI) calcd for C₄₂H₃₄IrN₃OSi: m/z 817.2100, Found: 817.2097

### Synthesis Example 8: Synthesis of Compound 300

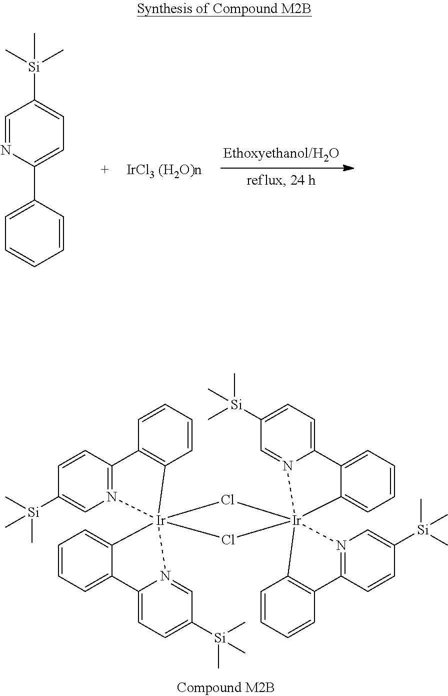

17.2 g (86%) of Compound M2B was obtained in the same manner as Compound M2A in Synthesis Example 1, except that 15.05 g (66.14 mmol) of 2-phenyl-5-(trimethylsilyl)pyridine and iridium 4.10 g (11.62 mmol) of chloride were respectively used instead of 14.66 g (94.44 mmol) of 2-phenylpyridine and 14.80 g (41.97 mmol) of iridium chloride.

Synthesis of Compound M1B

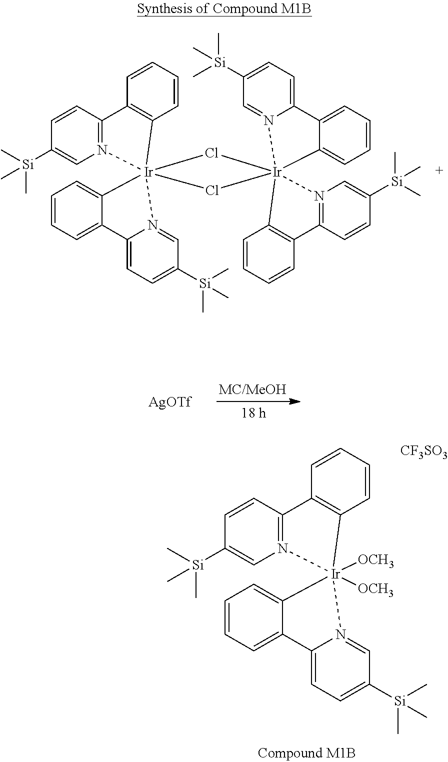

Compound M1B was obtained in the same manner as Compound M1A in Synthesis Example 1, except that 4.76 g (3.5 mmol) of Compound M2B was used instead of 6.01 g (5.60 mmol) of Compound M2A.

Synthesis of Compound 300

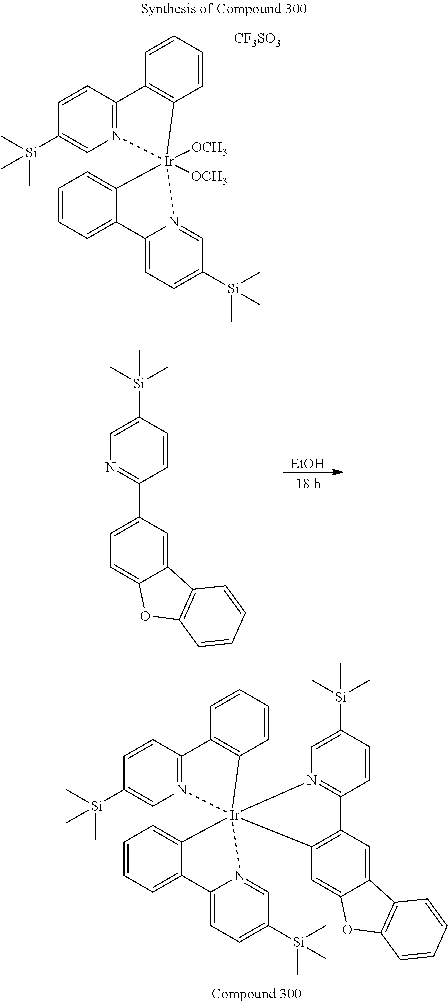

1.3 g (19%) of Compound 300 was obtained in the same manner as Compound 1 in Synthesis Example 1, except that 6.00 g (6.99 mmol) of Compound M1B and 2.66 g (8.39 mmol) of Compound A1 were respectively used instead of 8 g (11.22 mmol) of Compound M1A and 4.46 g (13.47 mmol) of Compound A1. The obtained product was confirmed by Mass and HPLC analysis.

HRMS (MALDI) calcd for C₄₈H₅₀IrN₃OSi₃: m/z 961.2891, Found: 961.2887

### Synthesis Example 9: Synthesis of Compound 305

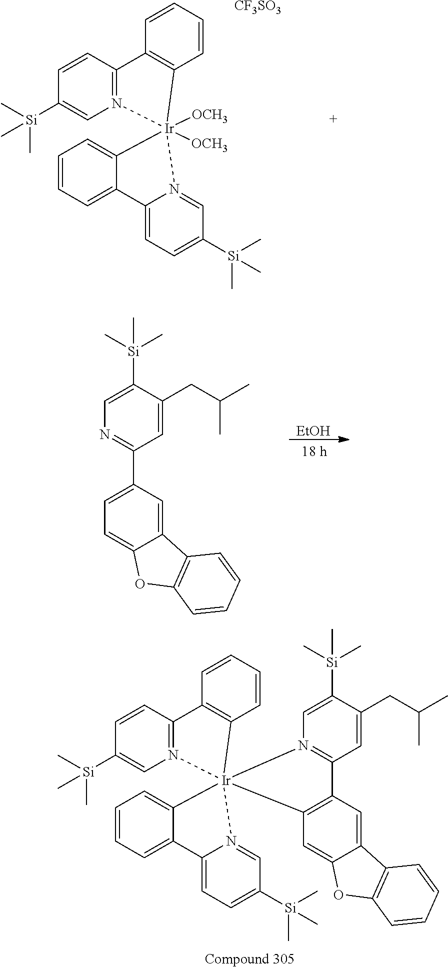

1.4 g (20%) of Compound 305 was obtained in the same manner as Compound 1 in Synthesis Example 1, except that 6.00 g (6.99 mmol) of Compound M1B and 3.14 g (8.39 mmol) of Compound C1 were respectively used instead of 8 g (11.22 mmol) of Compound M1A and 4.46 g (13.47 mmol) of Compound A1. The obtained product was confirmed by Mass and HPLC analysis.

HRMS (MALDI) calcd for C₅₂H₅₈IrN₃OSi₃: m/z 1017.3517, Found: 1017.3512

### Synthesis Example 10: Synthesis of Compound 322

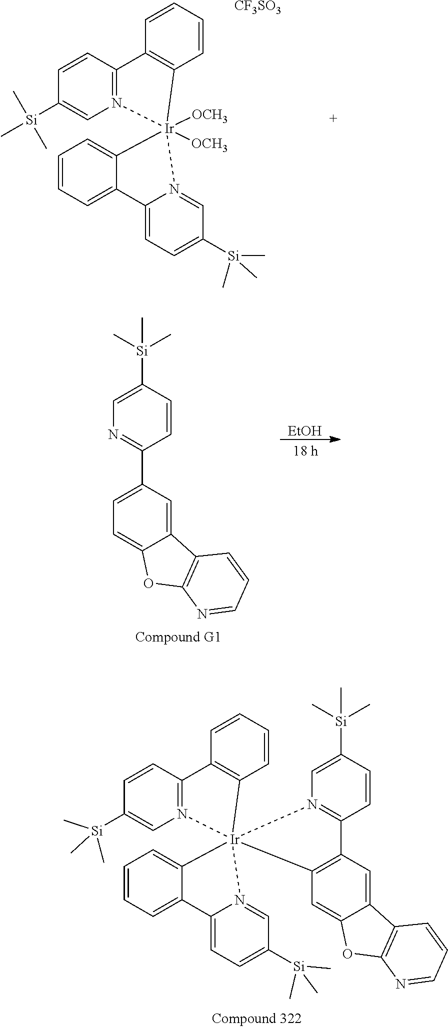

1.1 g (16%) of Compound 322 was obtained in the same manner as Compound 1 in Synthesis Example 1, except that 6.00 g (6.99 mmol) of Compound M1B and 2.78 g (8.74 mmol) of Compound G1 were respectively used instead of 8 g (11.22 mmol) of Compound M1A and 4.46 g (13.47 mmol) of Compound A1. The obtained product was confirmed by Mass and HPLC analysis.

HRMS (MALDI) calcd for C₄₇H₄₉IrN₄OSi₃: m/z 962.2843, Found: 962.2843

### Synthesis Example 11: Synthesis of Compound 327

Synthesis of Compound M2C

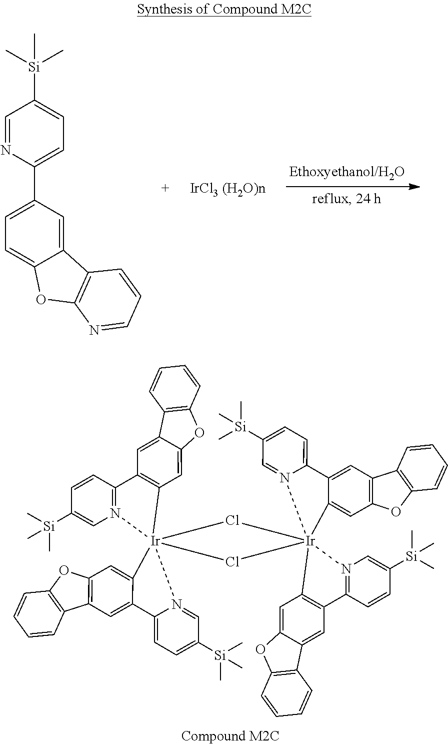

8.3 g (83%) of Compound M2C was obtained in the same manner as Compound M2A in Synthesis Example 1, except that 8.3 g (26.15 mmol) of Compound A1 and 4.10 g (11.62 mmol) of iridium chloride were respectively used instead of 14.66 g (94.44 mmol) of 2-phenylpyridine and 14.80 g (41.97 mmol) of iridium chloride.

Synthesis of Compound M1C

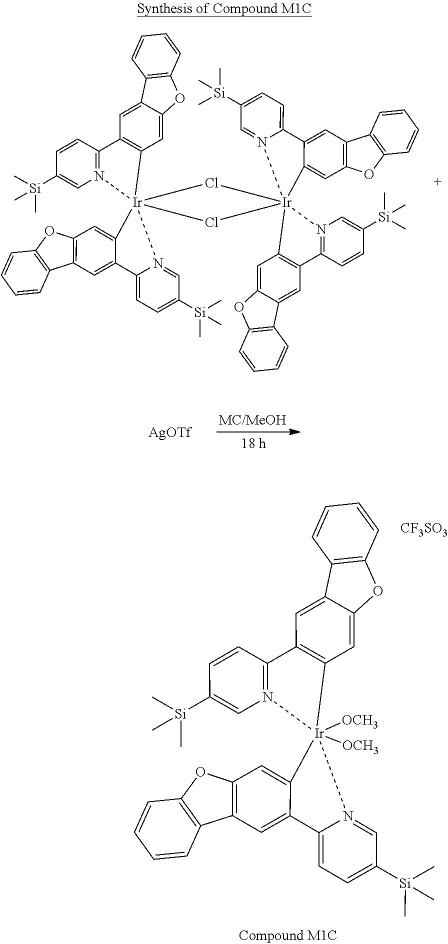

Compound M1C was obtained in the same manner as Compound M1A in Synthesis Example 1, except that 4.973 g (2.89 mmol) of Compound M2C was used instead of 6.01 g (5.60 mmol) of Compound M2A.

Synthesis of Compound 327

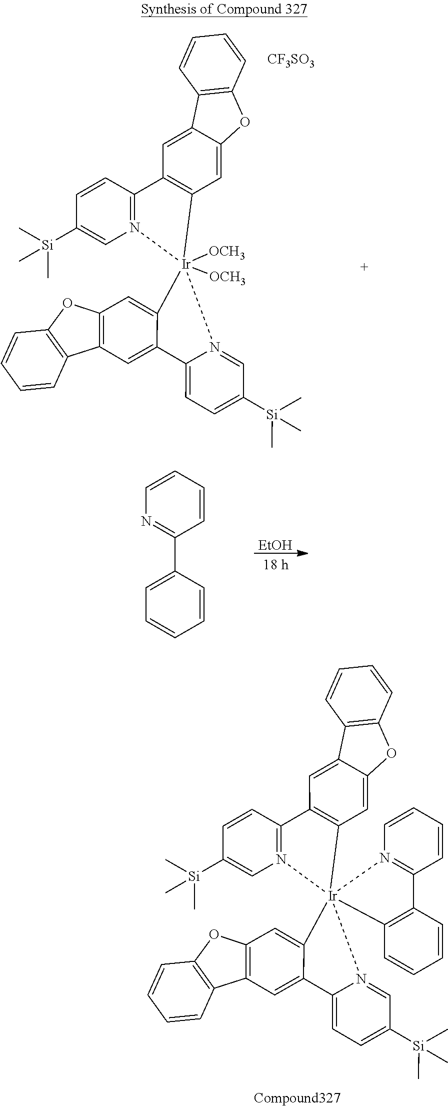

1.4 g (25%) of Compound 327 was obtained in the same manner as Compound 1 in Synthesis Example 1, except that 6.00 g (5.78 mmol) of Compound M1C and 1.08 g (6.94 mmol) of phenylpyridine were respectively used instead of 8 g (11.22 mmol) of Compound M1A and 4.46 g (13.47 mmol) of Compound A1. The obtained product was confirmed by Mass and HPLC analysis.

HRMS (MALDI) calcd for C₅₁H₄₄IrN₃O₂Si₂: m/z 979.2601, Found: 979.2603

### Synthesis Example 12: Synthesis of Compound A-1

Reaction Scheme (131)12

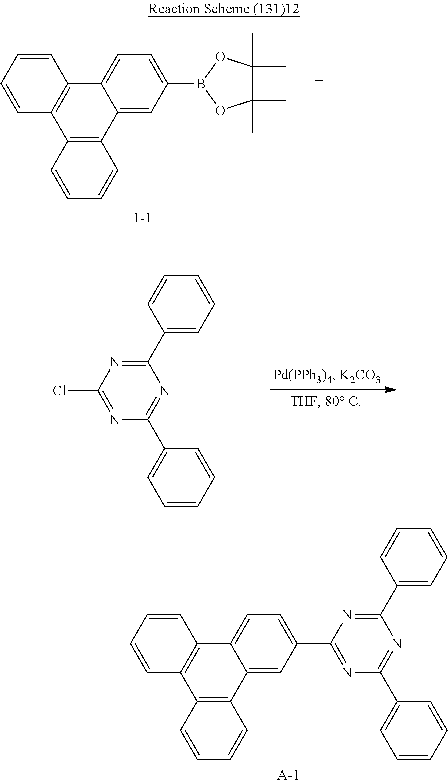 [SMILES: CC1(C)OB(c2ccc3c4ccccc4c4ccccc4c3c2)OC1(C)C ; Clc1nc(-c2ccccc2)nc(-c2ccccc2)n1 ; c1ccc(-c2nc(-c3ccccc3)nc(-c3ccc4c5ccccc5c5ccccc5c4c3)n2)cc1 ; "A-1"]

Under a nitrogen atmosphere, 20 g (56.5 mmol) of Intermediate 1-1 was dissolved in 0.2 liters (L) of tetrahydrofuran (THF), and 15.1 g (56.5 mmol) of 2-chloro-4,6-diphenyl-1,3,5-triazine and 0.65 g (0.57 mmol) of tetrakis(triphenylphosphine)palladium were added thereto and the reaction mixture was agitated. 19.5 g (141 mmol) of potassium carbonate saturated in water was added thereto, and the resultant was heated at a temperature 80° C. for 20 hours under reflux. After the reaction was completed, water was added to the reaction solution and the mixture was extracted by using dichloromethane (DCM). Then, an anhydrous magnesium sulfate (MgSO₄) was added thereto to perform dehydration. The resultant was filtered and concentrated under reduced pressure. The obtained residual was separated and purified by flash column chromatography to obtain 22.1 g (85%) of Compound A-1.

HRMS (70 eV, EI+): m/z calcd for C₃₃H₂₁N₃: 459.1735, found: 459.

Elemental Analysis: C, 86%; H, 5%

### Synthesis Example 13: Synthesis of Compound A-13

Reaction Scheme (131)13

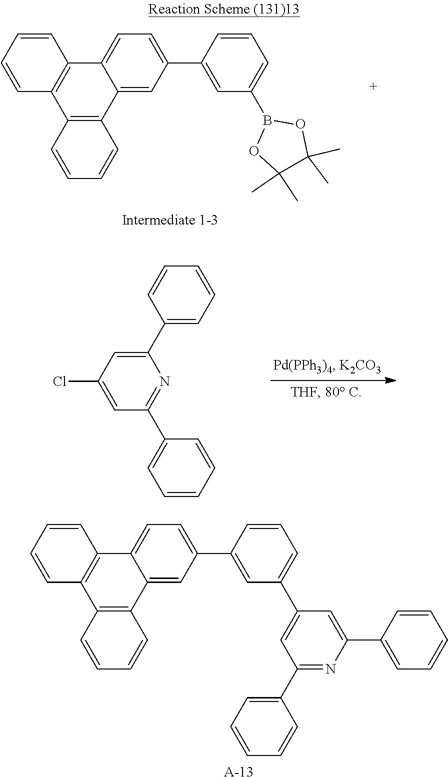

Under a nitrogen atmosphere, 20 g (46.5 mmol) of Intermediate 1-3 was dissolved in 0.2 L of tetrahydrofuran (THF), and 12.4 g (46.5 mmol) of 4-chloro-2,6-diphenylpyridine and 0.54 g (0.47 mmol) of tetrakis(triphenylphosphine)palladium were added thereto and the reaction mixture was agitated. 16.1 g (116 mmol) of potassium carbonate saturated in water was added thereto, and the resultant was heated at a temperature of 80° C. for 17 hours under reflux. After the reaction was completed, water was added to the reaction solution and the mixture was extracted by using dichloromethane (DCM). Then, an anhydrous magnesium sulfate (MgSO₄) was added thereto to perform dehydration. The resultant was filtered and concentrated under reduced pressure. The obtained residual was separated and purified by flash column chromatography to obtain 18.9 g (76%) of Compound A-13.

HRMS (70 eV, EI+): m/z calcd for C₄₁H₂₇N: 533.2143, found: 533.

Elemental Analysis: C, 92%; H, 5%

### Synthesis Example 14: Synthesis of Compound A-14

Reaction Scheme (131)14

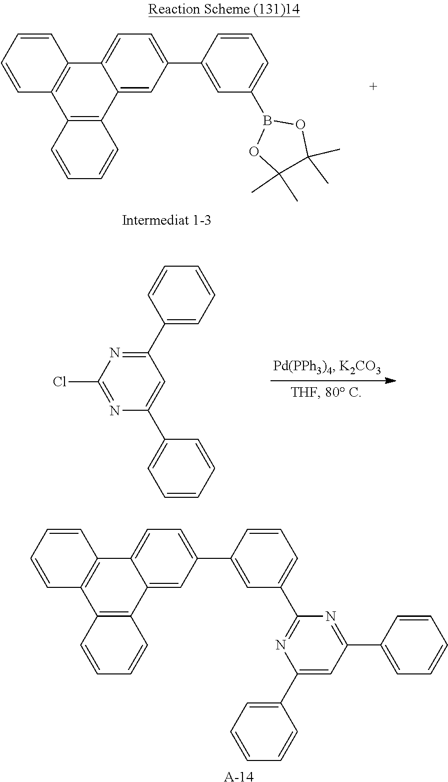

Under a nitrogen atmosphere, 20 g (46.5 mmol) of Intermediate 1-3 was dissolved in 0.2 L of tetrahydrofuran (THF), and 12.4 g (46.5 mmol) of 2-chloro-4,6-diphenylpyrimidine and 0.54 g (0.47 mmol) of tetrakis(triphenylphosphine)palladium were added thereto and the reaction mixture was agitated. Then, 16.1 g (116 mmol) of potassium carbonate saturated in water was added thereto, and the resultant was heated at a temperature of 80° C. for 15 hours under reflux. After the reaction was completed, water was added to the reaction solution and the mixture was extracted by using dichloromethane (DCM). Then, an anhydrous magnesium sulfate (MgSO₄) was added thereto to perform dehydration. The resultant was filtered and concentrated under reduced pressure. The obtained residual was separated and purified by flash column chromatography to obtain 20.4 g (82%) of Compound A-14.

HRMS (70 eV, EI+): m/z calcd for C₄₀H₂₆N₂: 534.2096, found: 534.

Elemental Analysis: C, 90%; H, 5%

### Synthesis Example 15: Compound A-15

Reaction Scheme (131)15

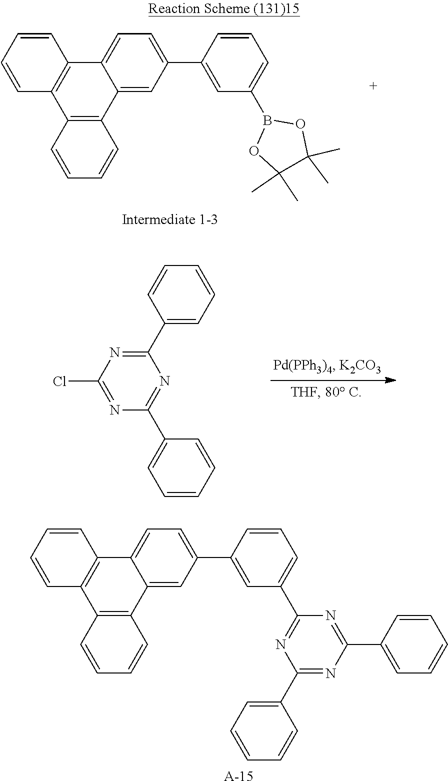

Under a nitrogen atmosphere, 20 g (46.5 mmol) of Intermediate 1-3 was dissolved in 0.2 L tetrahydrofuran (THF), and 12.4 g (46.5 mmol) of 2-chloro-4,6-diphenyl-1,3,5-triazine and 0.54 g (0.47 mmol) of triphenylphosphine)palladium were added thereto and the reaction mixture was agitated. 16.1 g (116 mmol) of potassium carbonate saturated in water was added thereto, and the resultant was heated at a temperature of 80° C. for 20 hours under reflux. After the reaction was completed, water was added to the reaction solution and the mixture was extracted by using dichloromethane (DCM). The anhydrous magnesium sulfate (MgSO₄) was added thereto to perform dehydration. The resultant was filtered and concentrated under reduced pressure. The obtained residual was separated and purified by flash column chromatography to obtain 21.2 g (85%) of Compound A-15.

HRMS (70 eV, EI+): m/z calcd for C₃₉H₂₅N₃: 535.2048, found: 535.

Elemental Analysis: C, 87%; H, 5%

### Synthesis Example 16: Synthesis of Compound A-24

Reaction Scheme (131)16

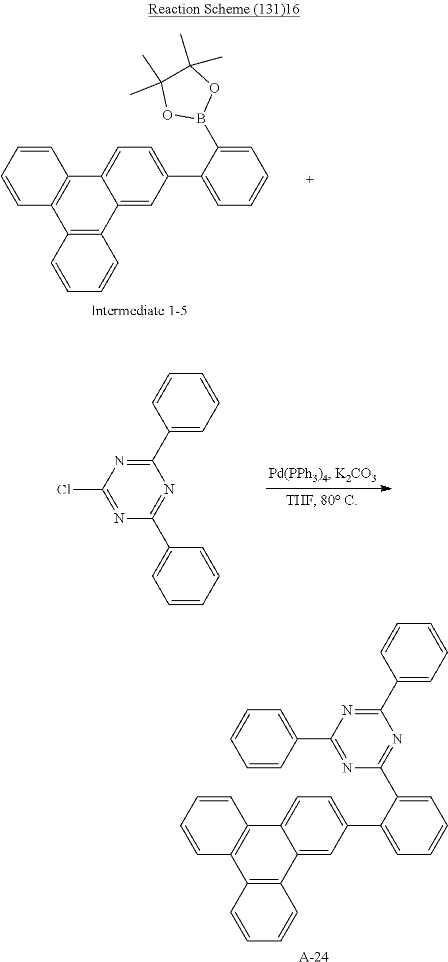

Under a nitrogen atmosphere, 20 g (46.5 mmol) of Intermediate 1-5 was dissolved in 0.2 L of tetrahydrofuran (THF), and 12.4 g (46.5 mmol) of 2-chloro-4,6-diphenyl-1,3,5-triazine and 0.54 g (0.47 mmol) of tetrakis(triphenylphosphine)palladium were added thereto and the reaction mixture was agitated. 16.1 g (116 mmol) of potassium carbonate saturated in water was added thereto, and the resultant was heated at a temperature of 80° C. for 27 hours under reflux. After the reaction was completed, water was added to the reaction solution and the mixture was extracted by using dichloromethane (DCM). Then, an anhydrous magnesium sulfate (MgSO₄) was added thereto to perform dehydration. The resultant was filtered and concentrated under reduced pressure. The obtained residual was separated and purified by flash column chromatography to obtain 19.7 g (79%) of Compound A-24.

HRMS (70 eV, EI+): m/z calcd for C₃₉H₂₅N₃: 535.2048, found: 535.

Elemental Analysis: C, 87%; H, 5%

### Synthesis Example 17: Synthesis of Compound A-33

Reaction Scheme (131)17

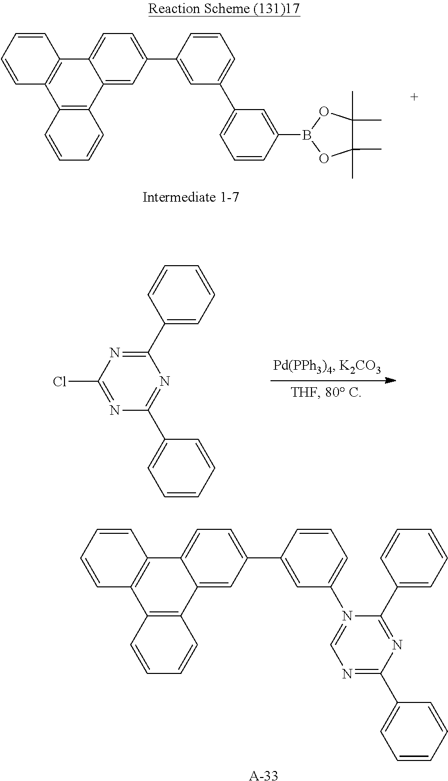

Under a nitrogen atmosphere, 20 g (39.5 mmol) of Intermediate 1-7 was dissolved in 0.2 L of tetrahydrofuran (THF), and 10.6 g (39.5 mmol) of 2-chloro-4,6-diphenyl-1,3,5-triazine and 0.46 g (0.4 mmol) of tetrakis(triphenylphosphine)palladium were added thereto and the reaction mixture was agitated. 13.6 g (98.8 mmol) of potassium carbonate saturated in water was added thereto, and the resultant was heated at a temperature of 80° C. for 23 hours under reflux. After the reaction was completed, water was added to the reaction solution and the mixture was extracted by using dichloromethane (DCM). Then, an anhydrous magnesium sulfate (MgSO₄) was added thereto to perform dehydration. The resultant was filtered and concentrated under reduced pressure. The obtained residual was separated and purified by flash column chromatography to obtain 17.9 g (74%) of Compound A-33.

HRMS (70 eV, EI+): m/z calcd for C₄₅H₂₉N₃: 611.2361, found: 611.

Elemental Analysis: C, 88%; H, 5%

### Synthesis Example 18: Synthesis of Compound A-69

Reaction Scheme (131)18

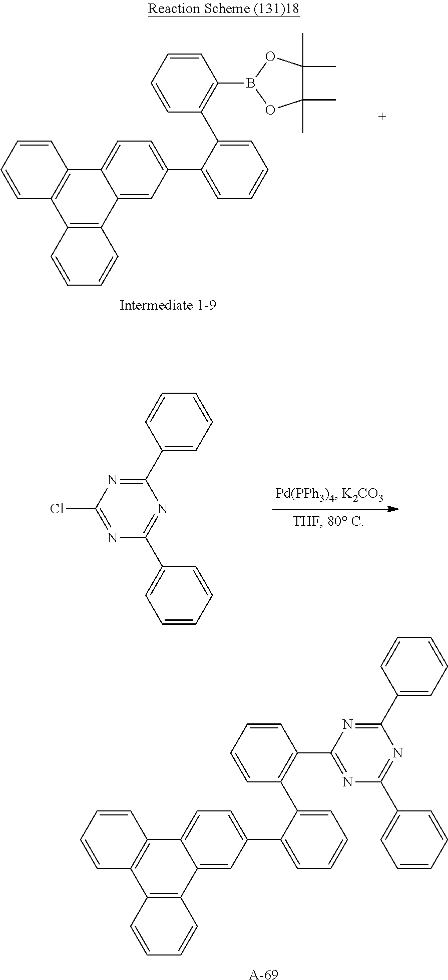 [SMILES: CC1(C)OB(c2ccccc2-c2ccccc2-c2ccc3c4ccccc4c4ccccc4c3c2)OC1(C)C ; Clc1nc(-c2ccccc2)nc(-c2ccccc2)n1 ; c1ccc(-c2nc(-c3ccccc3)nc(-c3ccccc3-c3ccccc3-c3ccc4c5ccccc5c5ccccc5c4c3)n2)cc1 ; "A-69"]

Under a nitrogen atmosphere, 20 g (39.5 mmol) of Intermediate 1-9 was dissolved in 0.2 L of tetrahydrofuran (THF), 10.6 g (39.5 mmol) of 2-chloro-4,6-diphenyl-1,3,5-triazine and 0.46 g (0.4 mmol) of tetrakis(triphenylphosphine)palladium were added thereto and the reaction mixture was agitated. 13.6 g (98.8 mmol) of potassium carbonate saturated in water was added thereto, and the resultant was heated at a temperature of 80° C. for 32 hours under reflux. After the reaction was completed, water was added to the reaction solution and the mixture was extracted by using dichloromethane (DCM). Then, an anhydrous magnesium sulfate (MgSO₄) was added thereto to perform dehydration. The resultant was filtered and concentrated under reduced pressure. The obtained residual was separated and purified by flash column chromatography to obtain 15.2 g (63%) of Compound A-69.

HRMS (70 eV, EI+): m/z calcd for C₄₅H₂₉N₃: 611.2361, found: 611.

Elemental Analysis: C, 88%; H, 5%

### Synthesis Example 19: Synthesis of Compound A-87

Reaction Scheme (131)19

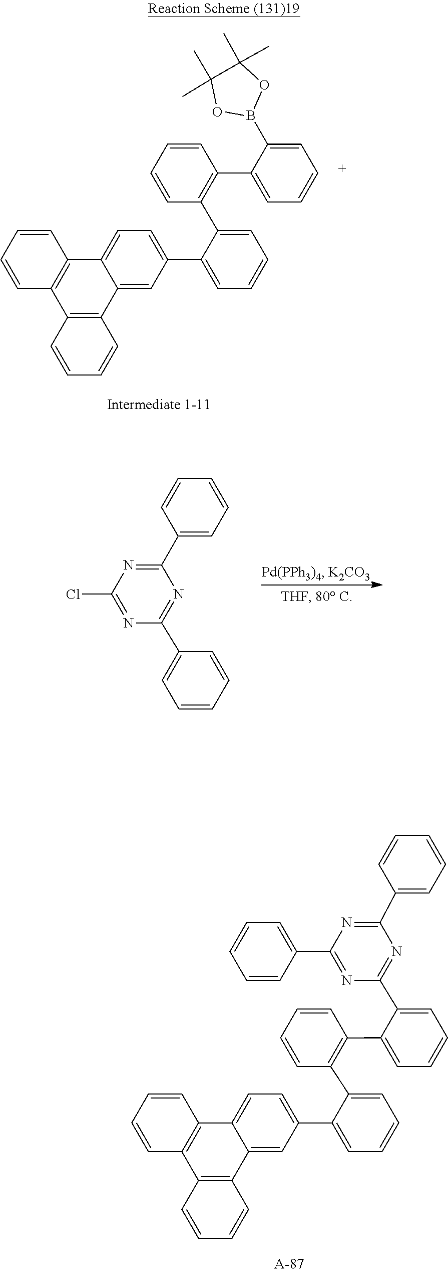 [SMILES: CC1(C)OB(c2ccccc2-c2ccccc2-c2ccccc2-c2ccc3c4ccccc4c4ccccc4c3c2)OC1(C)C ; Clc1nc(-c2ccccc2)nc(-c2ccccc2)n1 ; c1ccc(-c2nc(-c3ccccc3)nc(-c3ccccc3-c3ccccc3-c3ccccc3-c3ccc4c5ccccc5c5ccccc5c4c3)n2)cc1 ; "A-87"]

Under a nitrogen atmosphere, 20 g (34.3 mmol) of Intermediate 1-11 was dissolved in 0.2 L of tetrahydrofuran (THF), 9.19 g (34.3 mmol) of 2-chloro-4,6-diphenyl-1,3,5-triazine and 0.4 g (0.34 mmol) of tetrakis(triphenylphosphine)palladium were added thereto and the reaction mixture was agitated. 11.9 g (85.8 mmol) of potassium carbonate saturated in water was added thereto, and the resultant was heated at a temperature of 80° C. for 29 hours under reflux. After the reaction was completed, water was added to the reaction solution and the mixture was extracted by using dichloromethane (DCM). Then, an anhydrous magnesium sulfate (MgSO₄) was added thereto to perform dehydration. The resultant was filtered and concentrated under reduced pressure. The obtained residual was separated and purified by flash column chromatography to obtain 16.3 g (69%) of Compound A-87.

HRMS (70 eV, EI+): m/z calcd for C₅₁H₃₃N₃: 687.2674, found: 687.

Elemental Analysis: C, 89%; H, 5%

### Synthesis Example 20: Synthesis of Compound C-10

Reaction Scheme (131)20

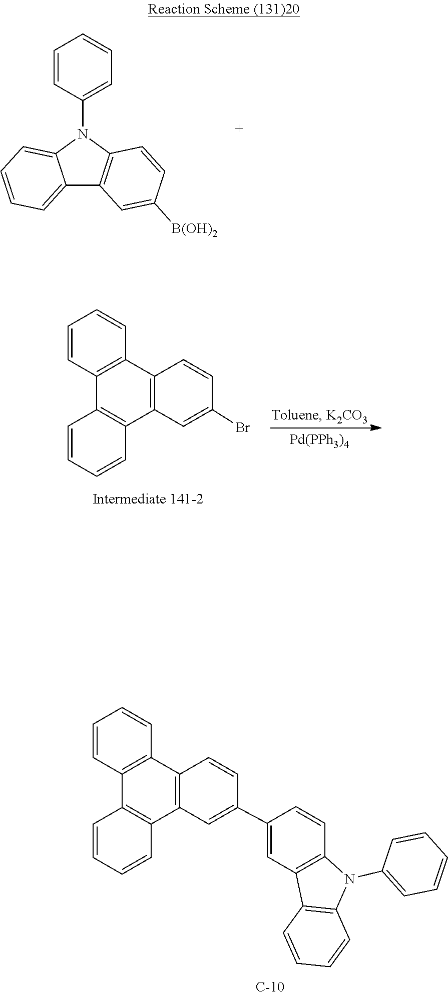

10 g (34.83 mmol) of phenylcarbazolyl boronic acid, 11.77 g (38.31 mmol) of Intermediate 141-2, 14.44 g (104.49 mmol) of potassium carbonate, 0.80 g (0.7 mmol) of tetrakis-(triphenylphosphine)palladium (0) were suspended in 140 ml of toluene and 50 ml of distilled water, and the suspended solution was refluxed and agitated for 12 hours. Then, the resultant was extracted with dichloromethane and distilled water, and the obtained organic layer was filtered through silica gel. Then, after removing an organic solution therefrom, a solid product was obtained through a silica gel column with hexane:dichloromethane=7:3 (volume to volume, v/v), and recrystallized with dichloromethane and n-hexane to obtain 14.4 g (88%) of Compound C-10.

HRMS (70 eV, EI+): m/z calcd for C₃₆H₂₃N: 469.18, found: 469

Elemental Analysis: C, 92%; H, 5%

### Synthesis Example 21: Synthesis of Compound B-10

Reaction Scheme (131)21

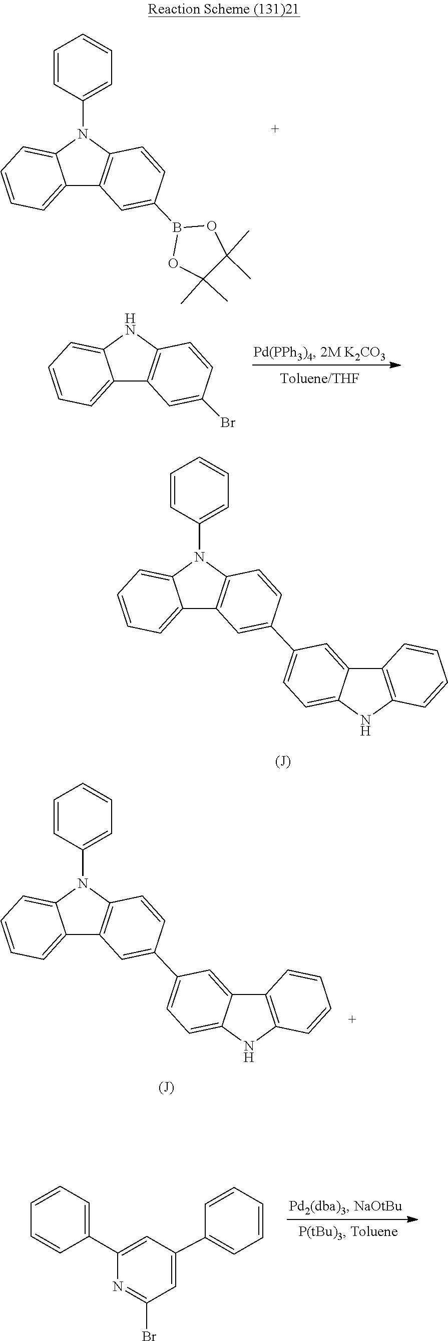

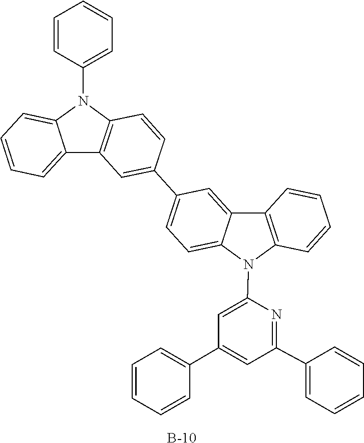

Synthesis of Compound (J)

26.96 g (81.4 mmol) of N-phenyl carbazole-3-boronic acid pinacolate and 23.96 g (97.36 mmol) of 3-bromo carbazole were mixed with 230 mL of tetrahydrofuran and 100 ml of 2 molar (M) potassium carbonate aqueous solution, and the mixture was heated under reflux in a nitrogen stream for 12 hours. After the reaction was completed, a solid produced by pouring methanol into the reactant was filtered and then dissolved in chlorobenzene, activated carbon and anhydrous magnesium sulfate were added thereto, and the mixture was agitated. The solution was filtered and recrystallized by using chlorobenzene and methanol to obtain 22.6 g (68%) of Compound (J).

HRMS (70 eV, EI+): m/z calcd for C₃₀H₂₀N₂: 408.16, found: 408

Elemental Analysis: C, 88%; H, 5%

Synthesis of Compound B-10

22.42 g (54.88 mmol) of Compound (J), 20.43 g (65.85 mmol) of 2-bromo-4,6-diphenylpyridine, and 7.92 g (82.32 mmol) of tertiary butoxy sodium were dissolved in 400 ml of toluene, and 1.65 g (1.65 mmol) of palladium dibenzylideneamine and 1.78 g (4.39 mmol) of tertiary butyl phosphorus were added in a dropwise fashion. The reaction solution was heated under reflux at 110° C. under a nitrogen stream for 12 hours. After the reaction was completed, a solid produced by pouring methanol into the reactant was filtered and then dissolved in chlorobenzene, activated carbon and anhydrous magnesium sulfate were added to thereto, and the mixture was agitated. The solution was filtered and recrystallized by using chlorobenzene and methanol to obtain 28.10 g (80%) of Compound B-10.

HRMS (70 eV, EI+): m/z calcd for C₄₇H₃₁N₃: 637.25, found: 637

Elemental Analysis: C, 89%; H, 5%

### Synthesis Example 22: Synthesis of Compound B-31

Reaction Scheme (131)22

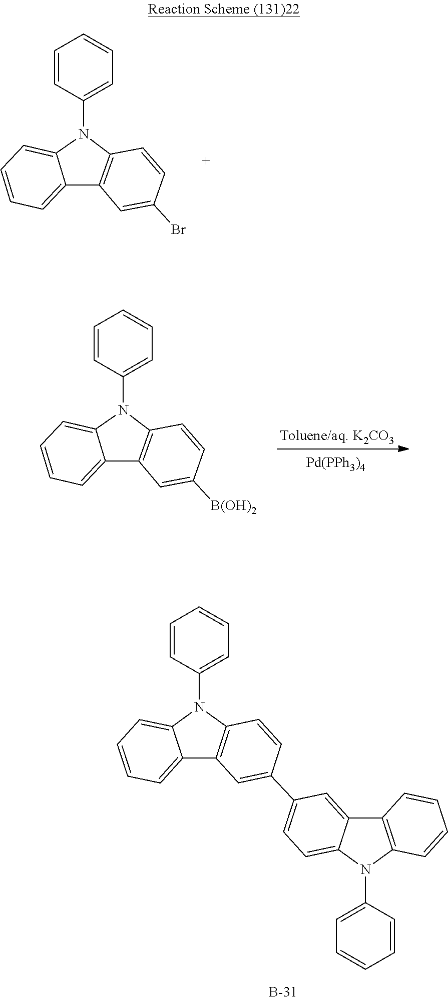

9.97 g (30.95 mmol) of phenylcarbazolyl bromide, 9.78 g (34.05 mmol) of phenylcarbazolyl boronic acid, 12.83 g (92.86 mmol) of potassium carbonate, and 1.07 g (0.93 mmol) of tetrakis-(triphenylphosphine)palladium (0) were suspended in 120 ml of toluene and 50 ml of distilled water, and the suspended solution was refluxed and agitated for 12 hours. Then, the resultant was extracted with dichloromethane and distilled water, and the organic layer was filtered through silica gel therefrom. Then, after removing an organic solution therefrom, a solid product obtained was recrystallized with dichloromethane and n-hexane, obtaining 13.8 g (92%) of Compound B-31.

HRMS (70 eV, EI+): m/z calcd for C₃₆H₂₄N₂: 484.19, found: 484

Elemental Analysis: C, 89%; H, 5%

Compound 23: Synthesis of Compound B-34

Reaction Scheme (131)23

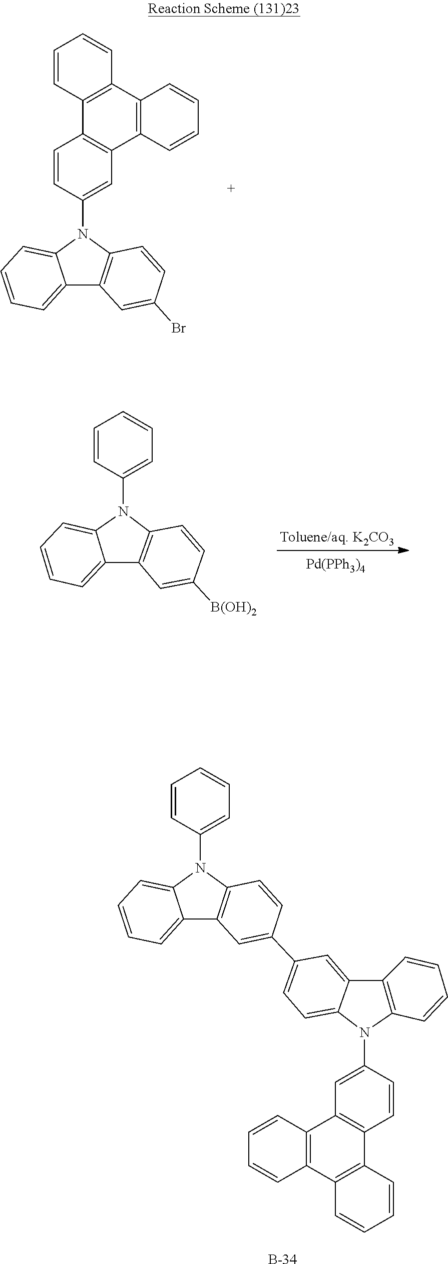

14.62 g (30.95 mmol) of triphenyl carbazolyl bromide, 9.78 g (34.05 mmol) of phenylcarbazolyl boronic acid, 12.83 g (92.86 mmol) of potassium carbonate, and 1.07 g (0.93 mmol) of tetrakis-(triphenylphosphine)palladium (0) were suspended in 120 ml of toluene and 50 ml of distilled water, and the suspended solution was refluxed and agitated for 12 hours. Then, the resultant was extracted with dichloromethane and distilled water, and the organic layer produced therein was filtered through silica gel. Then, a solid product obtained after removing an organic solution therefrom was recrystallized with dichloromethane and n-hexane to obtain 16.7 g (85%) of Compound B-34.

HRMS (70 eV, EI+): m/z calcd for C₄₇H₂₉N₂: 621.23, found: 621

Elemental Analysis: C, 91%; H, 5%

### Synthesis Example 24: Synthesis of Compound B-43

Reaction Scheme (131)24

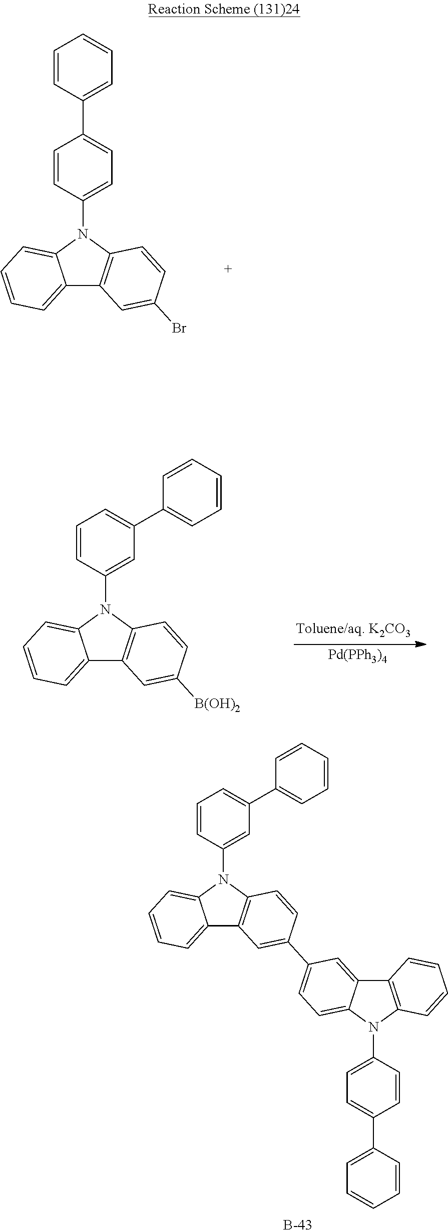

12.33 g (30.95 mmol) of biphenylcarbazolyl bromide, 12.37 g (34.05 mmol) of biphenylcarbazolyl boronic acid, 12.83 g (92.86 mmol) of potassium carbonate, and 1.07 g (0.93 mmol) of tetrakis-(triphenylphosphine)palladium (0) were suspended in 120 ml of toluene and 50 ml of distilled water, and the suspended solution was refluxed and agitated for 12 hours. Then, the resultant was extracted with dichloromethane and distilled water, and the organic layer produced therein was filtered through silica gel. Then, a solid product obtained after removing an organic solution therefrom was recrystallized with dichloromethane and n-hexane to obtain 18.7 g (92%) of Compound B-43.

HRMS (70 eV, EI+): m/z calcd for C₄₈H₃₂N₂: 636.26, found: 636

Elemental Analysis: C, 91%; H, 5%

### Synthesis Example 25: Synthesis of Compound B-114

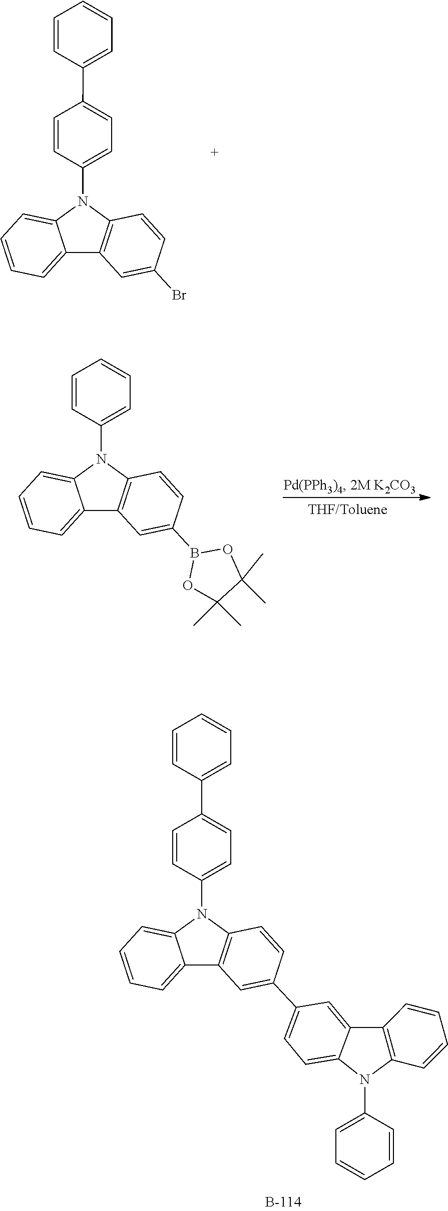 [SMILES: Brc1ccc2c(c1)c1ccccc1n2-c1ccc(-c2ccccc2)cc1 ; CC1(C)OB(c2ccc3c(c2)c2ccccc2n3-c2ccccc2)OC1(C)C ; c1ccc(-c2ccc(-n3c4ccccc4c4cc(-c5ccc6c(c5)c5ccccc5n6-c5ccccc5)ccc43)cc2)cc1]

Under a nitrogen atmosphere, 20.00 g (50.21 mmol) of 3-bromo-N-biphenylcarbazole and 18.54 g (50.21 mmol) of N-phenylcarbazole-3-boronic ester were mixed with 175 mL of tetrahydrofuran:toluene (1:1) and 75 mL of 2 M potassium carbonate solution in a 500-mL round-bottom flask with an agitator, 2.90 g (2.51 mmol) of tetrakis-(triphenylphosphine)palladium (0) was added thereto, and the mixture was heated under reflux under a nitrogen stream for 12 hours. After the reaction was completed, a solid produced by pouring the reactant into methanol was filtered, thoroughly washed with water and methanol, and dried. The result obtained therefrom was heated and dissolved in 700 mL of chlorobenzene, the solution was filtered through silica gel, and the solvent was completely removed. Then, the resultant was heated and dissolved in 400 mL of chlorobenzene and then recrystallized to obtain 19.15 g (68%) of Compound A2.

calcd. C₄₂H₂₈N₂: C, 89.97; H, 5.03; N, 5.00; found: C, 89.53; H, 4.92; N, 4.89

### Synthesis Example 26: Synthesis of Compound B-14

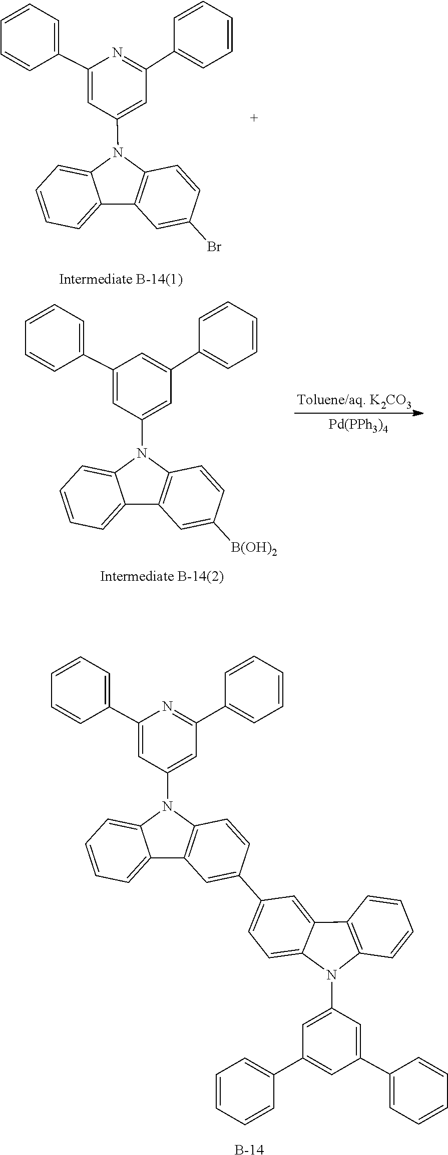

Compound B-14 was synthesized in the same manner as in Synthesis Example 24, except that Intermediate B-14(1) (3-bromo-9-(2,6-diphenylpyridin-4-yl)-9H-carbazole) and Intermediate B-14(2) ((9-([1,1′:3′,1″-terphenyl]-5′-yl)-9H-carbazol-3-yl)boronic acid) were respectively used instead of biphenylcarbazolyl bromide and biphenylcarbazolylboronic acid.

### Synthesis Example 27: Synthesis of Compound B-15

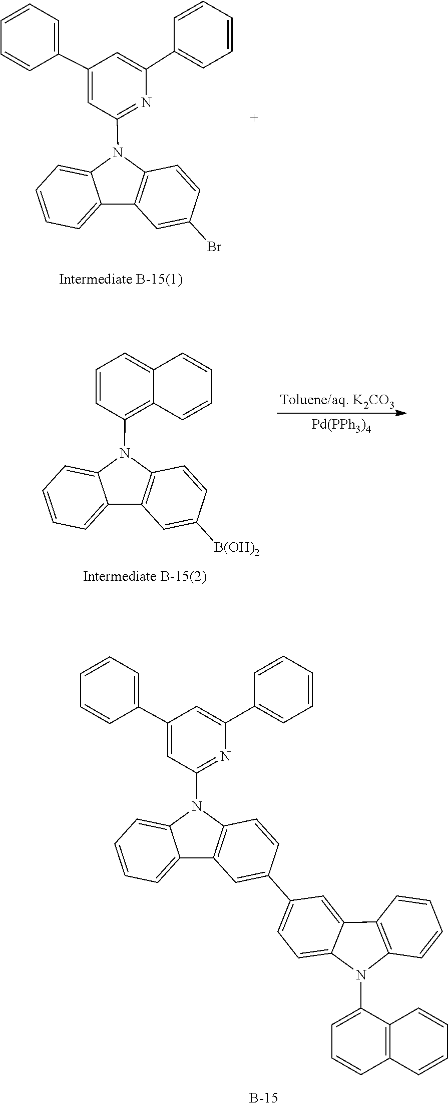

Compound B-15 was synthesized in the same manner as in Synthesis Example 24, except that Intermediate B-15(1) (3-bromo-9-(4,6-diphenylpyridin-2-yl)-9H-carbazole) and Intermediate B-15(2) ((9-(naphthalen-1-yl)-9H-carbazol-3-yl)boronic acid) were respectively used instread of biphenylcarbazolyl bromide and biphenylcarbazolylboronic acid.

### Example 1

An anode was prepared by cutting an ITO glass substrate, on which an ITO electrode was deposited, to a size of 50 mm×50 mm×0.5 mm (mm=millimeter), ultrasonically cleaning the ITO glass substrate (anode) using acetone, iso-propyl alcohol, and pure water each for 15 minutes, and exposing the ITO glass substrate (anode) to irradiation of UV for 30 minutes and ozone to clean.

Compound HT3 and F4-TCNQ (a centration of F4-TCNQ in a hole injection layer was 3 percent by weight, wt %) were co-deposited on the anode at a deposition rate of 1 Angstrom per second (Å/sec) to form a hole injection layer having a thickness of 100 Angstroms (Å), and Compound HT3 was deposited on the hole injection layer at a deposition rate of 1 Å/sec to form a hole transport layer having a thickness of 1,660 Å.

A dopant and a host were co-deposited on the hole transport layer to form an emission layer having a thickness of 400 Å.

Materials used as the dopant and the host in Examples and Comparative Examples, content of the dopant in the emission layer, a weight ratio between two hosts when two host were used are shown in Table 2 below.

Compound ET16 and LiQ were co-deposited on the emission layer at a weight ratio of 5:5 to form an electron transport layer having a thickness of 360 Å. Then, LiQ was deposited on the electron transport layer to form an electron injection layer having a thickness of 5 Å, and an A1 electrode having a thickness of 800 Å was formed on the electron injection layer, thereby completing the manufacture of an organic light-emitting device.

### Evaluation Example 1: Evaluation on Characteristics of Organic Light-Emitting Devices

The driving voltage, current efficiency, power efficiency, and lifespan (T₉₅) of the organic light-emitting devices manufactured in Examples 1 to 8 and Comparative Examples 1 and 2 were evaluated. Results thereof are shown in Table 2. This evaluation was performed using a current-voltage meter (Keithley 2400) and a luminescence meter (Minolta Cs-1,000A), and the lifespan (T₉₅) (at 6000 nit) was evaluated by measuring the amount of time that elapsed until luminance was reduced to 95% of the initial brightness of 100%.

TABLE 2

| | | | | | Driving | Current | Power | Lifespan |
|---|---|---|---|---|---|---|---|---|
| | | Host | | Weight ratio of | Voltage | Efficiency | Efficiency | (hr) |
| | Dopant | host 1 | host 2 | host 1:host 2 | (V) | (cd/A) | (lm/W) | (T₉₅) |
| Example 1 | Compound 1 | Compound | Compound | 5:5 | 4.3 | 53 | 38.7 | 250 |
| | (10 wt %) | B-31 | A-14 | | | | | |
| Example 2 | Compound 1 | Compound | Compound | 5:5 | 4.1 | 55 | 42.1 | 265 |
| | (10 wt %) | B-43 | A-15 | | | | | |
| Example 3 | Compound 3 | Compound | Compound | 5:5 | 4.5 | 50 | 34.9 | 190 |
| | (10 wt %) | B-43 | B-14 | | | | | |
| Example 4 | Compound 3 | Compound | Compound | 5:5 | 4.2 | 51 | 38.1 | 200 |
| | (10 wt %) | B-43 | B-15 | | | | | |
| Example 5 | Compound | Compound | Compound | 5:5 | 4.1 | 55 | 42.1 | 290 |
| | 146 | B-31 | A-14 | | | | | |
| | (10 wt %) | | | | | | | |
| Example 6 | Compound | Compound | Compound | 5:5 | 4.0 | 56 | 44.0 | 320 |
| | 146 | B-43 | A-15 | | | | | |
| | (10 wt %) | | | | | | | |
| Example 7 | Compound | Compound | Compound | 5:5 | 3.7 | 51 | 43.3 | 215 |
| | 322 | B-31 | A-14 | | | | | |
| | (10 wt %) | | | | | | | |
| Example 8 | Compound | Compound | Compound | 5:5 | 3.6 | 53 | 46.2 | 240 |
| | 322 | B-43 | A-15 | | | | | |
| | (10 wt %) | | | | | | | |
| Comparative | Ir(ppy)₃ | Compound | Compound | 5:5 | 4.8 | 45 | 29.4 | 110 |
| Example 1 | (10 wt %) | B-31 | B-14 | | | | | |
| Comparative | Comparative | Compound | Compound | 5:5 | 4.6 | 48 | 32.8 | 135 |
| Example 2 | Compound A | B-31 | B-14 | | | | | |
| | (10 wt %) | | | | | | | |

Compound 1 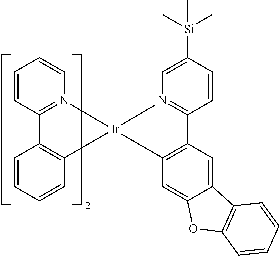
Compound 3 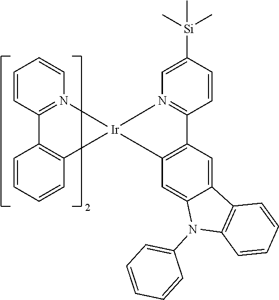
Compound 8 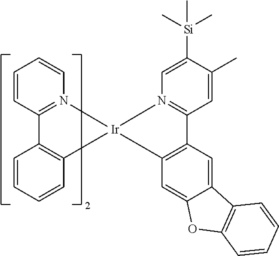
Compound 22 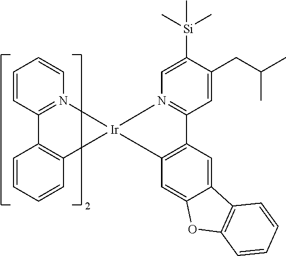
Compound 32 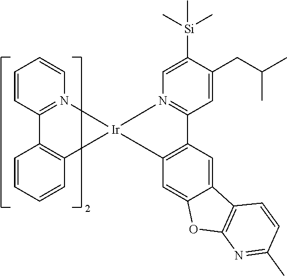
Compound 35 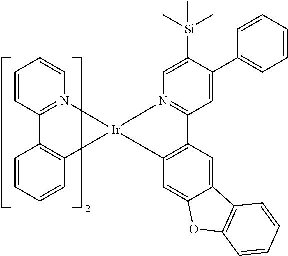
Compound 146 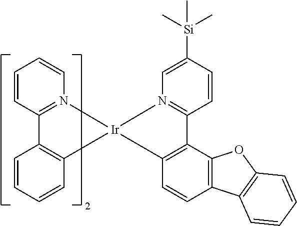
Compound 300 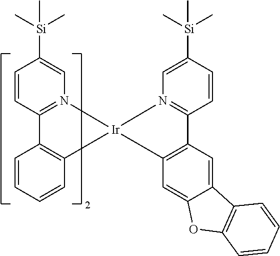
Compound 305 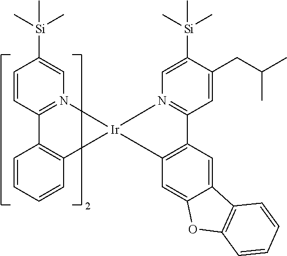
Compound 322 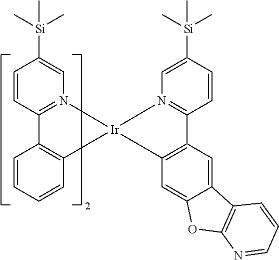
Compound 327 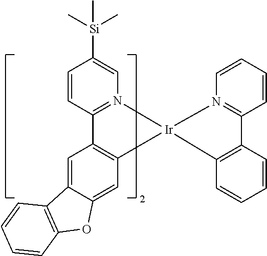
Comparative Compound A 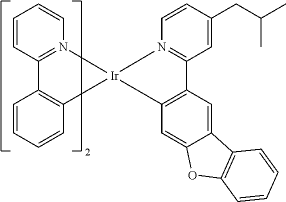
A-14 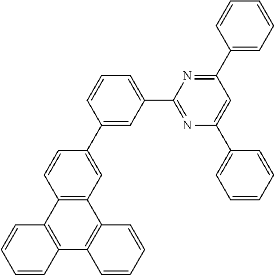 [SMILES: c1ccc(-c2cc(-c3ccccc3)nc(-c3cccc(-c4ccc5c6ccccc6c6ccccc6c5c4)c3)n2)cc1]
A-15 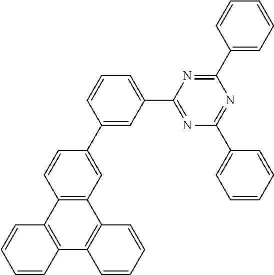 [SMILES: c1ccc(-c2nc(-c3ccccc3)nc(-c3cccc(-c4ccc5c6ccccc6c6ccccc6c5c4)c3)n2)cc1]
B-14 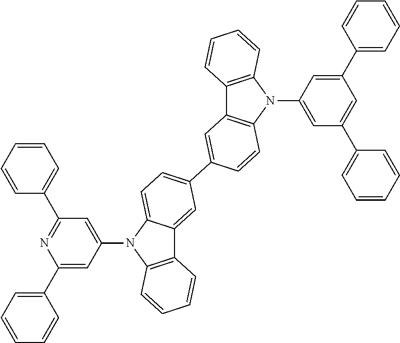 [SMILES: c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1]
B-15 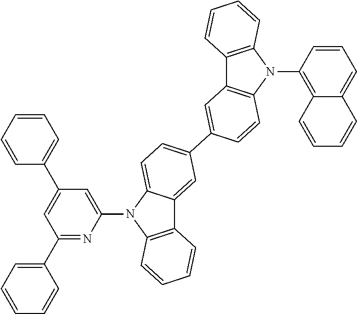 [SMILES: c1ccc(-c2cc(-c3ccccc3)nc(-n3c4ccccc4c4cc(-c5ccc6c(c5)c5ccccc5n6-c5cccc6ccccc56)ccc43)c2)cc1]
B-31 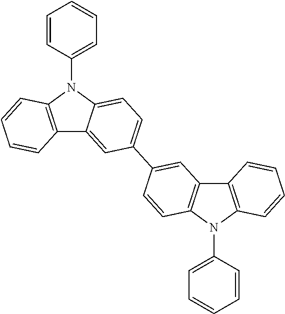 [SMILES: c1ccc(-n2c3ccccc3c3cc(-c4ccc5c(c4)c4ccccc4n5-c4ccccc4)ccc32)cc1]
B-43

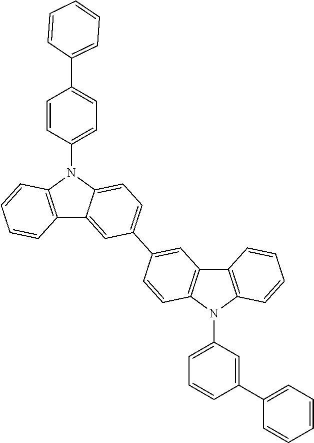 [SMILES: c1ccc(-c2ccc(-n3c4ccccc4c4cc(-c5ccc6c(c5)c5ccccc5n6-c5cccc(-c6ccccc6)c5)ccc43)cc2)cc1]

Referring to table 2, it was confirmed that the organic light-emitting devices of Examples 1 to 8 had excellent driving voltage, current efficiency, power efficiency, and lifespan characteristics, compared to those of Comparative Examples 1 and 2.

According to one or more embodiments, the organic light-emitting device may have low driving voltage, high efficiency, and long lifespan characteristics.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the present disclosure as defined by the following claims.

What is claimed is:

1. An organic light-emitting device comprising:
a first electrode;
a second electrode facing the first electrode; and
an organic layer disposed between the first electrode and the second electrode,
wherein the organic layer comprises an emission layer,
wherein the emission layer further comprises:
i) an organometallic compound represented by Formula 1; and
ii) at least one selected from a first compound represented by Formula 51, a second compound represented by Formula 61, a third compound represented by Formula 81, and a fourth compound represented by Formula 91: 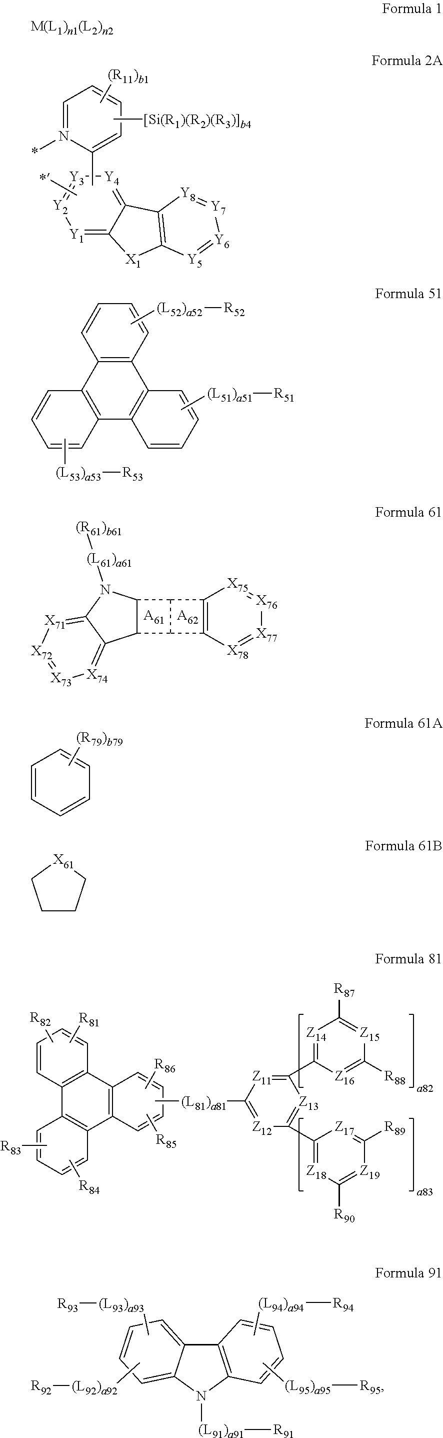
wherein
M in Formula 1 is selected from iridium (Ir), platinum (Pt), osmium (Os), titanium (Ti), zirconium (Zr), hafnium (Hf), europium (Eu), terbium (Tb), thulium (Tm), and rhodium (Rₕ),
in Formula 1, L₁ is a ligand represented by Formula 2A, and n1 is 1, 2, or 3,
wherein when n1 is two or more, two or more groups L₁ are identical to or different from each other,
in Formula 1, L₂ is selected from a monovalent organic ligand, a divalent organic ligand, a trivalent organic ligand, and a tetravalent organic ligand, and n2 is 0, 1, 2, 3, or 4, wherein when n2 is two or more, two or more groups L₂ are identical to or different from each other,
L₁ and L₂ in Formula 1 are different from each other,
R₁ to R₃ in Formula 2A are each independently selected from a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, and —Si(Q₅₁)(Q₅₂)(Q₅₃),
X₁ in Formula 2A is selected from O, S, S(═O)₂, N(R₂₁), and Si(R₂₂)(R₂₃),
in Formula 2A, Y₁ is N, C(R₄₁), or C linked to a pyridine ring, Y₂ is N, C(R₄₂) or C linked to a pyridine ring, Y₃ is N, C(R₄₃) or C linked to a pyridine ring, Y₄ is N, C(R₄₄), or C linked to a pyridine ring, Y₅ is N or C(R₄₅), Y₆ is N or C(R₄₆), Y₇ is N or C(R₄₇), Y₈ is N or C(R₄₈), one selected from Y₁ to Y₄ is C linked to a pyridine ring, and at least one of Y₁ to Y₈ is N,
R₁₁, R₂₁ to R₂₃, and R₄₁ to R₄₈ in Formula 2A are each independently selected from hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), and —P(═O)(Q₈)(Q₉),
two or more groups R₁₁ are optionally linked to form a saturated or unsaturated C₄-C₆₀ ring,
two or more selected from R₄₁ to R₄₄ are optionally linked to form a saturated or unsaturated C₄-C₆₀ ring,
two or more selected from R₄₅ to R₄₈ are optionally linked to form a saturated or unsaturated C₄-C₆₀ ring,
in Formula 2A, b1 is an integer selected from 0 to 3, and b4 is an integer selected from 1 to 4,
* and *′ in Formula 2A each indicate a binding site to M in Formula 1,
ring A₈₁ in Formula 61 is represented by Formula 61A,
ring A₆₂ in Formula 61 is represented by Formula 61B,
X₆₁ in Formula 61B is N-[(L₆₂)ₐ₆₂-(R₆₂)_{b62}], S, O, S(═O), S(═O)₂, C(═O), C(R₆₃)(R₆₄), Si(R₆₃)(R₆₄), P(R₆₃), P(═O)(R₆₃), or C═N(R₆₃),
in Formula 61, X₇₁ is C(R₇₁) or N, X₇₂ is C(R₇₂) or N, X₇₃ is C(R₇₃) or N, X₇₄ is C(R₇₄) or N, X₇₅ is C(R₇₅) or N, X₇₆ is C(R₇₈) or N, X₇₇ is C(R₇₇) or N, and X₇₈ is C(R₇₈) or N,
L₅₁ to L₅₃, L₈₁, L₆₂, and L₉₁ to L₉₅ in Formulae 51, 61, 61B, and 91 are each independently selected from a single bond, a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,
a51 to a53, a61, a62, and a91 to a95 in Formulae 51, 61, 61B, and 91 are each independently an integer selected from 1 to 5,
R₅₁ to R₅₃, R₆₁ to R₆₄, R₇₁ to R₇₉, and R₉₁ to R₉₅ in Formulae 51, 61, 61A, 61B, and 91 are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁₁)(Q₁₂), —Si(Q₁₃)(Q₁₄)(Q₁₅), and —B(Q₁₆)(Q₁₇),
b61, b62, and b79 in Formulae 61A and 61B are each independently an integer selected from 0 to 3,
in Formula 81, Z₁₁ is N or C(Rₐ), Z₁₂ is N or C(R_{b}), Z₁₃ is N or C(R_{c}), Z₁₄ is N or C(R_{d}), Z₁₅ is N or C(Rₑ), Z₁₆ is N or C(R_{f}), Z₁₇ is N or C(R_{g}), Z₁₈ is N or C(Rₕ), Z₁₉ is N or C(Rᵢ), and at least one selected from Z₁₁ to Z₁₉ is N,
R₈₁ to R₉₀ and Rₐ to Rᵢ in Formula 81 are each independently selected from hydrogen, deuterium, a substituted or unsubstituted C₁-C₁₀ alkyl group, and a substituted or unsubstituted C₆-C₁₂ aryl group,
a total number of 6-membered rings substituted with a triphenylene core in Formula 81 is six or less,
L₈₁ in Formula 81 is selected from a substituted or unsubstituted phenylene group, a substituted or unsubstituted biphenylene group, and a substituted or unsubstituted terphenylene group,
a81 to a83 in Formula 81 are each independently 0 or 1,
in Formula 81, a81+a82+a83≥1,
at least one selected from L₉₁ to L₉₅ and R₉₁ to R₉₅ in Formula 91 comprises a carbazole ring or a triphenylene ring,
at least one substituent selected from a substituent(s) of the substituted C₁-C₂₀ alkylene group, the substituted C₂-C₂₀ alkenylene group, the substituted C₃-C₁₀ cycloalkylene group, the substituted C₁-C₁₀ heterocycloalkylene group, the substituted C₃-C₁₀ cycloalkenylene group, the substituted C₁-C₁₀ heterocycloalkenylene group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, the substituted monovalent non-aromatic condensed heteropolycyclic group, the substituted phenylene group, the substituted biphenylene group, and the substituted terphenylene group is selected from:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and
—N(Q₃₁)(Q₃₂), —Si(Q₃₃)(Q₃₄)(Q₃₅), and —B(Q₃₆)(Q₃₇),
wherein Q₁ to Q₉, Q₁₁ to Q₁₉, to Q₂₉, Q₃₁ to Q₃₉, and Q₅₁ to Q₅₃ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

2. The organic light-emitting device of claim 1, wherein,
in Formula 1,
M is Ir and n1+n2 is 3; or
M is Pt and n1+n2 is 2, and
the organometallic compound represented by Formula 1 is electrically neutral.

3. The organic light-emitting device of claim 1, wherein
Y₁ or Y₃ in Formula 2A is N;
Y₅ or Y₆ in Formula 2A is N; or
one or two selected from Y₁, Y₃, Y₅, and Y₆ in Formula 2A are N.

4. The organic light-emitting device of claim 1, wherein
R₁₁, R₂₁ to R₂₃, and R₄₁ to R₄₈ in Formula 2A are each independently selected from:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, C₁-C₂₀ alkyl group, and a C₁-C₂₀ alkoxy group;
a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group; and
—B(Q₆)(Q₇) and —P(═O)(Q₈)(Q₉),
wherein Q₆ to Q₉ are each independently selected from:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;
an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and
an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium, a C₁-C₁₀ alkyl group, and a phenyl group.

5. The organic light-emitting device of claim 1, wherein
R₁₁, R₂₁ to R₂₃, and R₄₁ to R₄₈ in Formula 2A are each independently selected from hydrogen, deuterium, —F, a cyano group, a nitro group, —SF₅, —CH₃, —CD₃, —CD₂H, —CDH₂, —CF₃, —CF₂H, —CFH₂, groups represented by Formulae 9-1 to 9-17, and groups represented by Formulae 10-1 to 10-30: 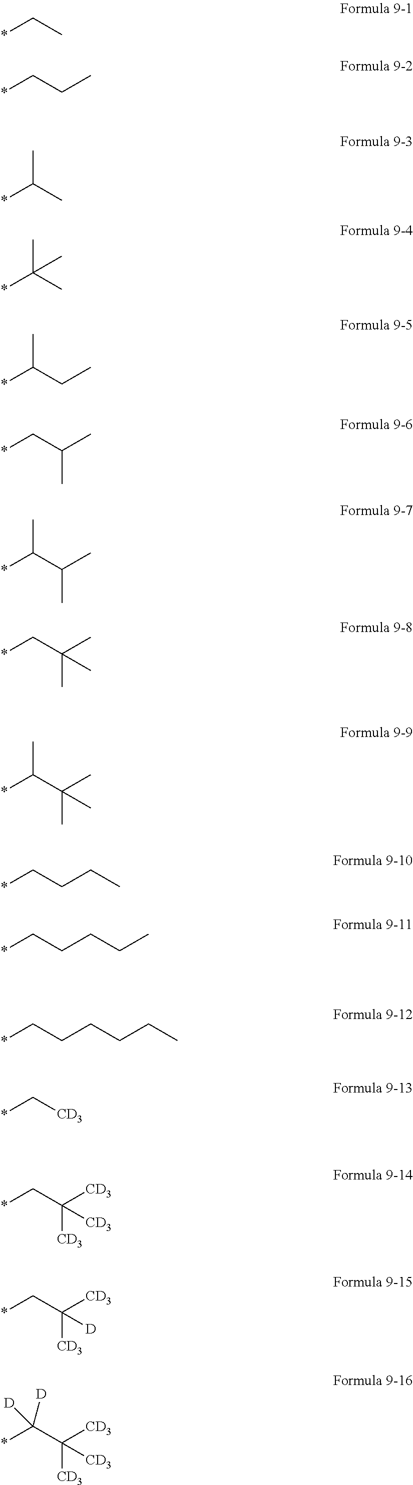 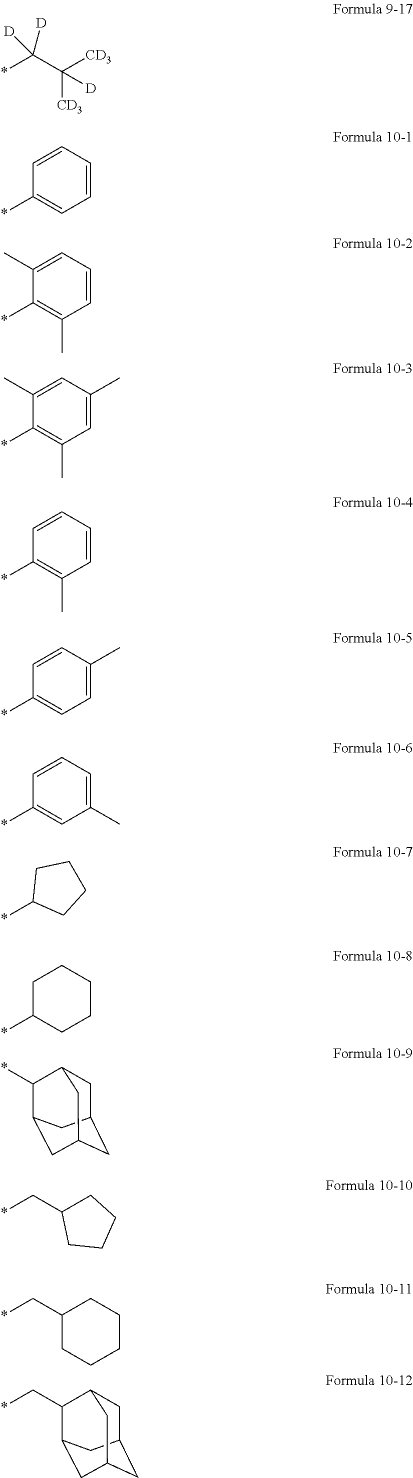 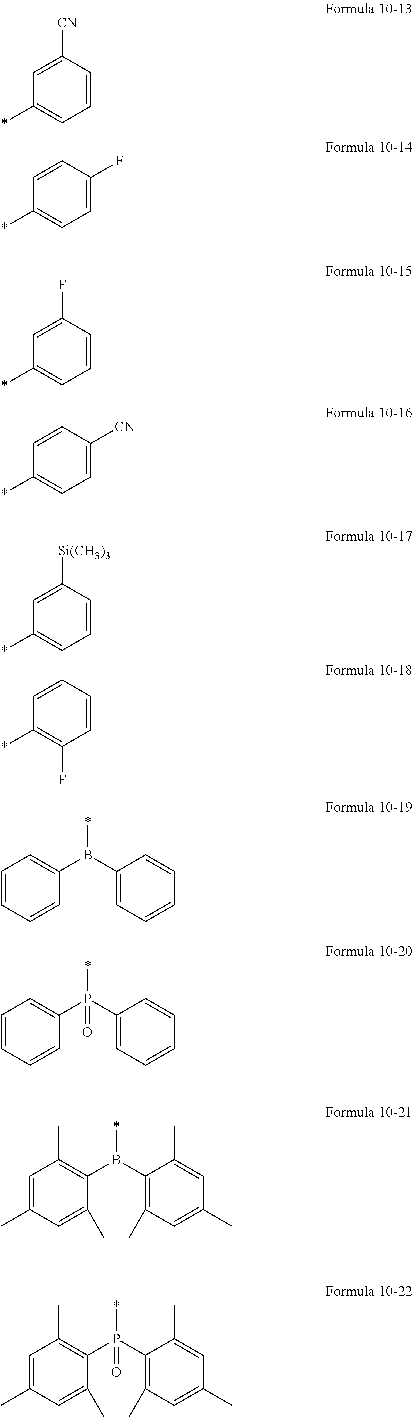 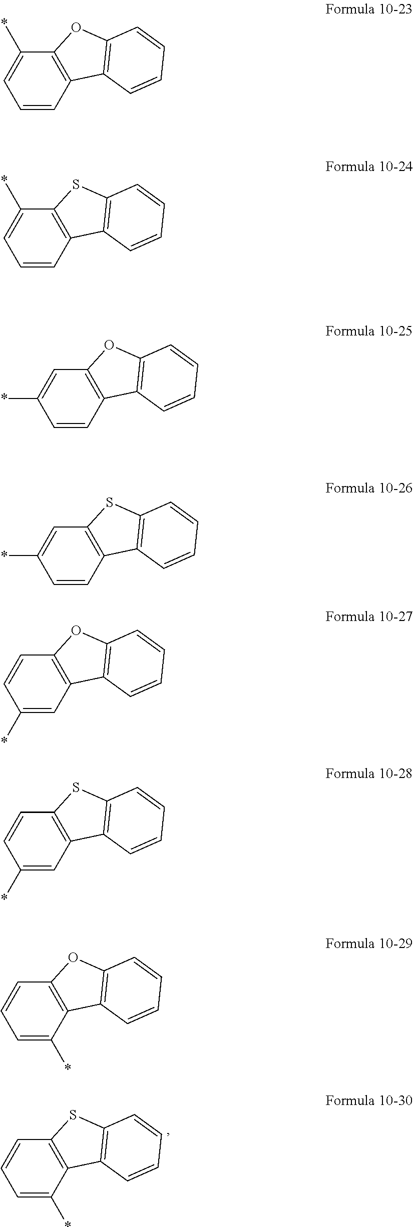
wherein * in Formulae 9-1 to 9-17 and 10-1 to 10-30 indicates a binding site to a neighboring atom.

6. The organic light-emitting device of claim 1, wherein
L₁ in Formula 1 is selected from ligands represented by Formulae 2A-1 to 2A-16: 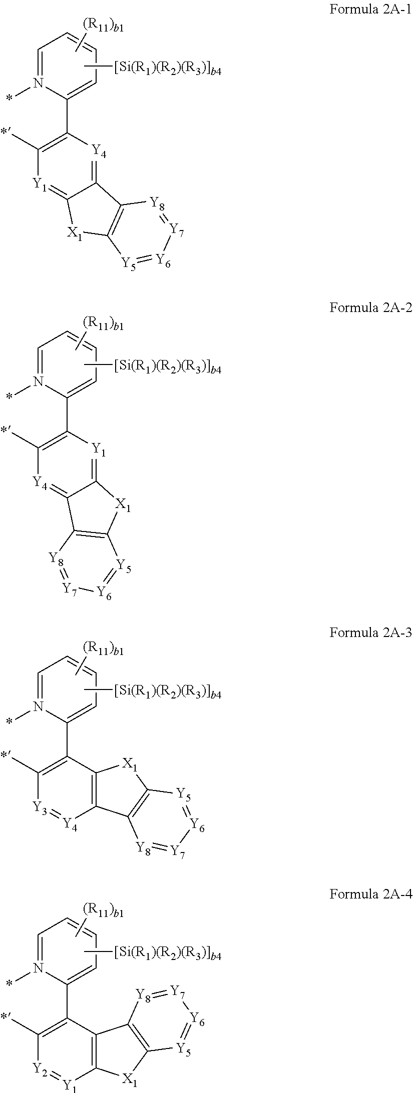 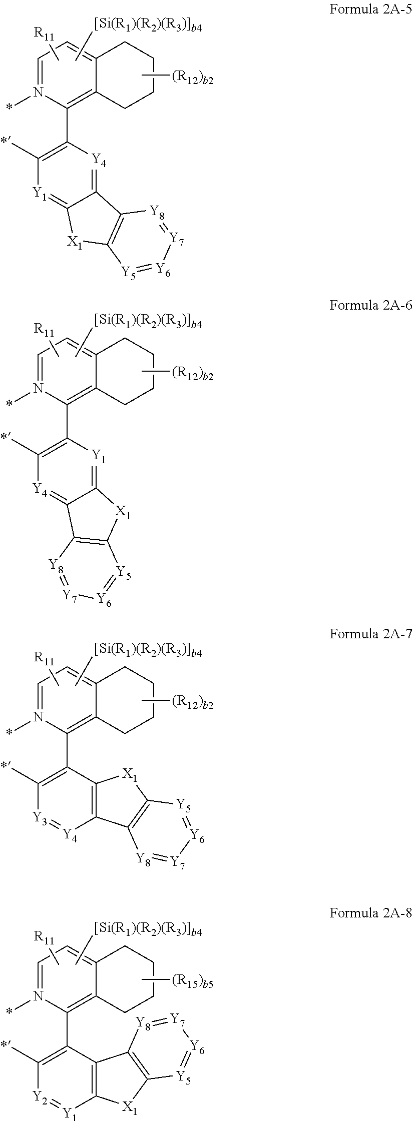 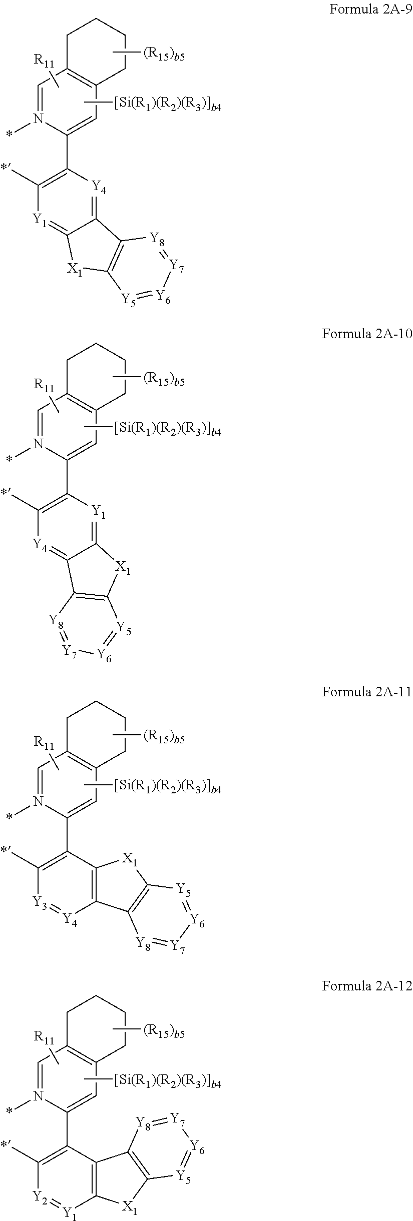 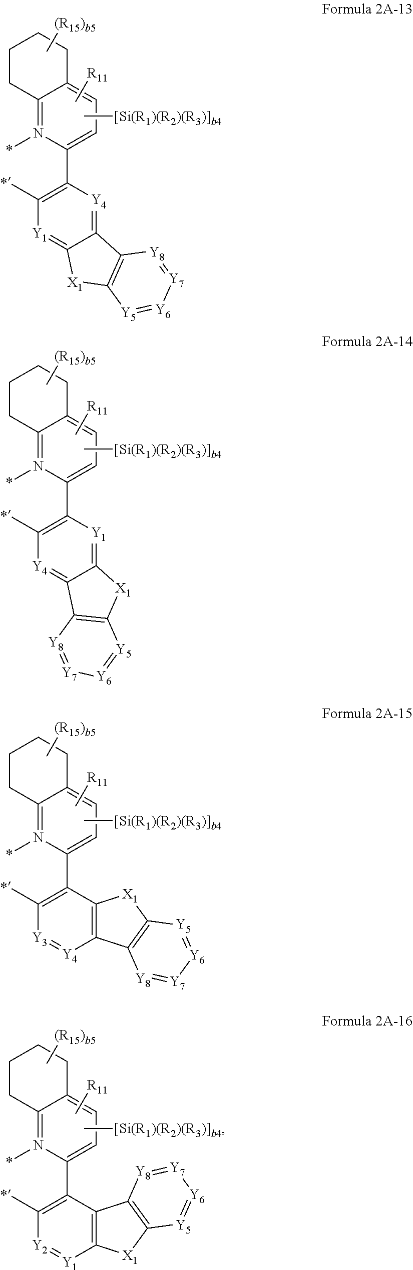
wherein, in Formulae 2A-1 to 2A-16,
R₁ to R₃, X₁, Y₁ to Y₈, R₁₁, b1, and b4 are the same as in claim 1,
R₁₅ is the same as in connection with R₁₁,
b5 is an integer selected from 0 to 8, and
* and *′ each indicate a binding site to M in Formula 1.

7. The organic light-emitting device of claim 6, wherein,
in Formulae 2A-1, 2A-2, 2A-4, 2A-5, 2A-6, 2A-8, 2A-9, 2A-10, 2A-12, 2A-13, 2A-14, and 2A-16, Y₁ is N, Y₄ is C(R₄₄), Y₅ is C(R₄₅), Y₆ is C(R₄₆), Y₇ is C(R₄₇), and Y₈ is C(R₄₈);
in Formulae 2A-1, 2A-2, 2A-4, 2A-5, 2A-6, 2A-8, 2A-9, 2A-10, 2A-12, 2A-13, 2A-14, and 2A-16, Y₁ is C(R₄₁), Y₄ is C(R₄₄), Y₅ is N, Y₆ is C(R₄₆), Y₇ is C(R₄₇), and Y₈ is C(R₄₈);
in Formulae 2A-1, 2A-2, 2A-4, 2A-5, 2A-6, 2A-8, 2A-9, 2A-10, 2A-12, 2A-13, 2A-14, and 2A-16, Y₁ is C(R₄₁), Y₄ is C(R₄₄), Y₅ is C(R₄₅), Y₆ is N, Y₇ is C(R₄₇), and Y₈ is C(R₄₈);
in Formulae 2A-1, 2A-2, 2A-4, 2A-5, 2A-6, 2A-8, 2A-9, 2A-10, 2A-12, 2A-13, 2A-14, and 2A-16, Y₁ is N, Y₄ is C(R₄₄), Y₅ is N, Y₆ is C(R₄₆), Y₇ is C(R₄₇), and Y₈ is C(R₄₈);
in Formulae 2A-1, 2A-2, 2A-4, 2A-5, 2A-6, 2A-8, 2A-9, 2A-10, 2A-12, 2A-13, 2A-14, and 2A-16, Y₁ is N, Y₄ is C(R₄₄), Y₅ is C(R₄₅), Y₆ is N, Y₇ is C(R₄₇), and Y₈ is C(R₄₈);
in Formulae 2A-3, 2A-7, 2A-11, and 2A-15, Y₃ is N, Y₄ is C(R₄₄), Y₅ is C(R₄₅), Y₆ is C(R₄₈), Y₇ is C(R₄₇), and Y₈ is C(R₄₈);
in Formulae 2A-3, 2A-7, 2A-11, and 2A-15, Y₃ is C(R₄₃), Y₄ is C(R₄₄), Y₅ is N, Y₆ is C(R₄₈), Y₇ is C(R₄₇), and Y₈ is C(R₄₈);
in Formulae 2A-3, 2A-7, 2A-11, and 2A-15, Y₃ is C(R₄₃), Y₄ is C(R₄₄), Y₅ is C(R₄₅), Ye is N, Y₇ is C(R₄₇), and Y₈ is C(R₄₈);
in Formulae 2A-3, 2A-7, 2A-11, and 2A-15, Y₃ is N, Y₄ is C(R₄₄), Y₅ is N, Y₆ is C(R₄₈), Y₇ is C(R₄₇), and Y₈ is C(R₄₈); or
in Formulae 2A-3, 2A-7, 2A-11, and 2A-15, Y₃ is N, Y₄ is C(R₄₄), Y₅ is C(R₄₅), Y₆ is N, Y₇ is C(R₄₇), and Y₈ is C(R₄₈).

8. The organic light-emitting device of claim 1, wherein
L₁ in Formula 1 is selected from groups represented by Formulae 2AA-1, 2AA-2, 2AA-3, 2AA-4, and 2AB: 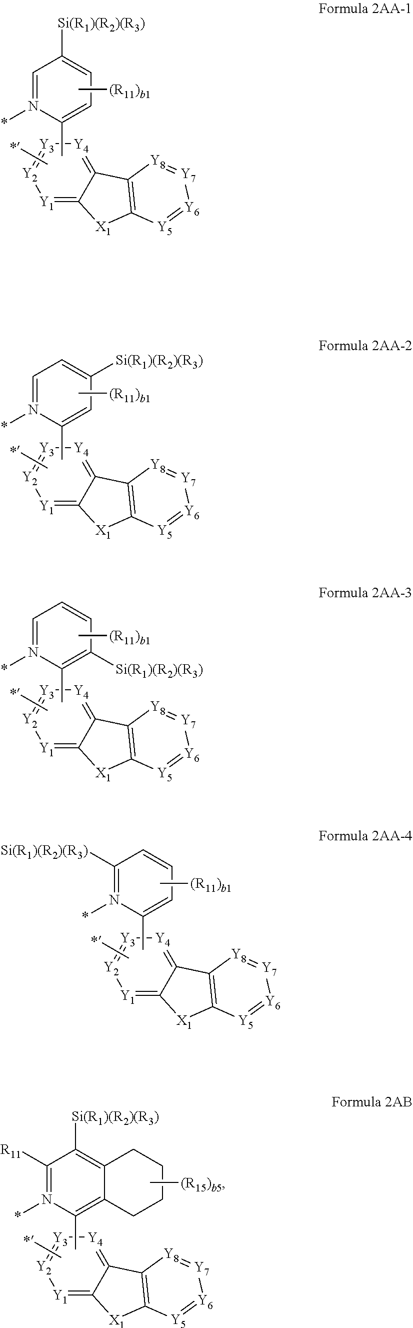
wherein, in Formulae 2AA-1, 2AA-2, 2AA-3, 2AA-4, and 2AB,
R₁ to R₃, Y₁ to Y₈, R₁₁, and b1 are the same as in claim 1,
R₁₅ is the same as in connection with R₁₁,
b5 is an integer selected from 0 to 8, and
* and *′ each indicate a binding site to M in Formula 1.

9. The organic light-emitting device of claim 1, wherein
L₁ in Formula 1 is selected from ligands represented by Formulae 2A(1) to 2A(40): 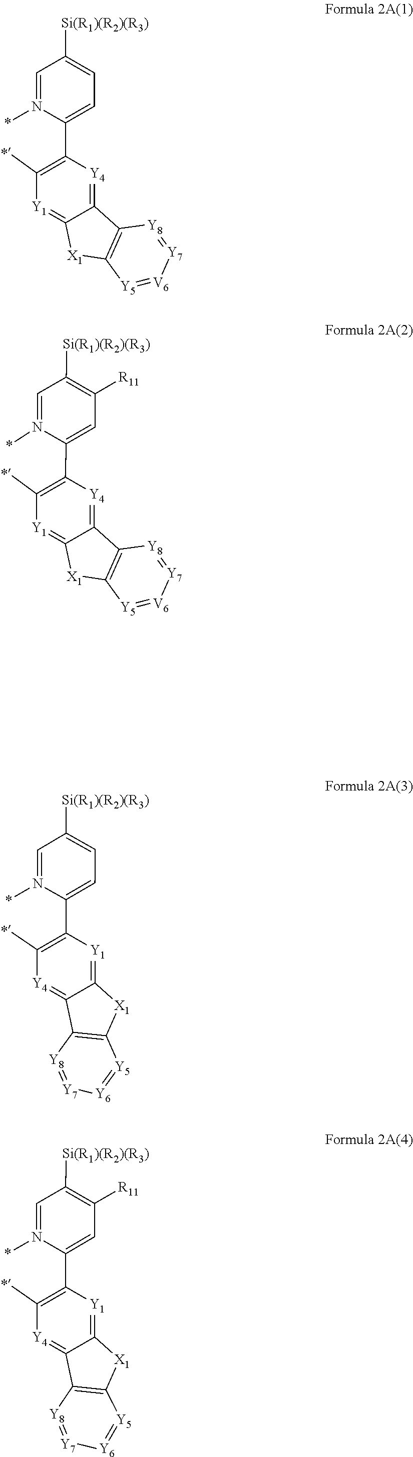 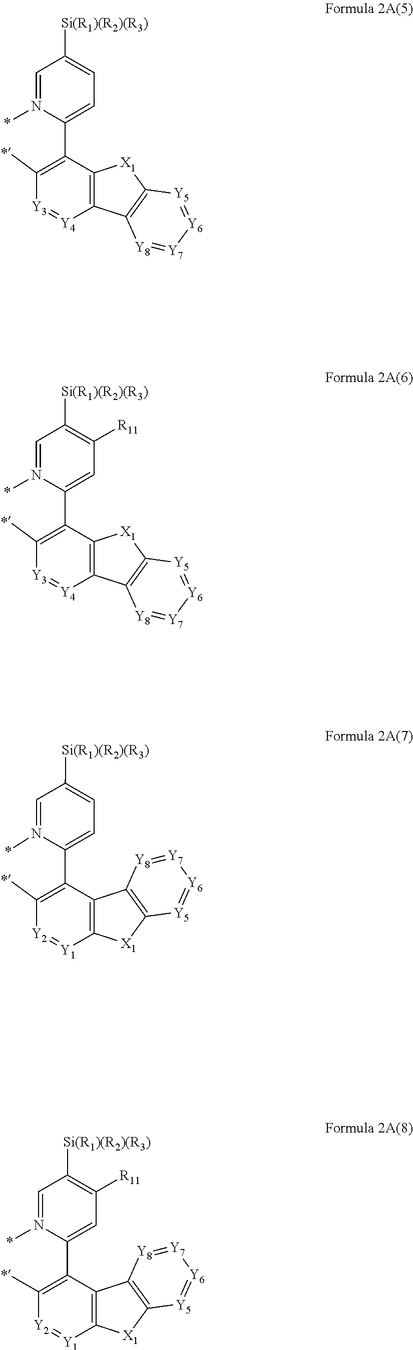 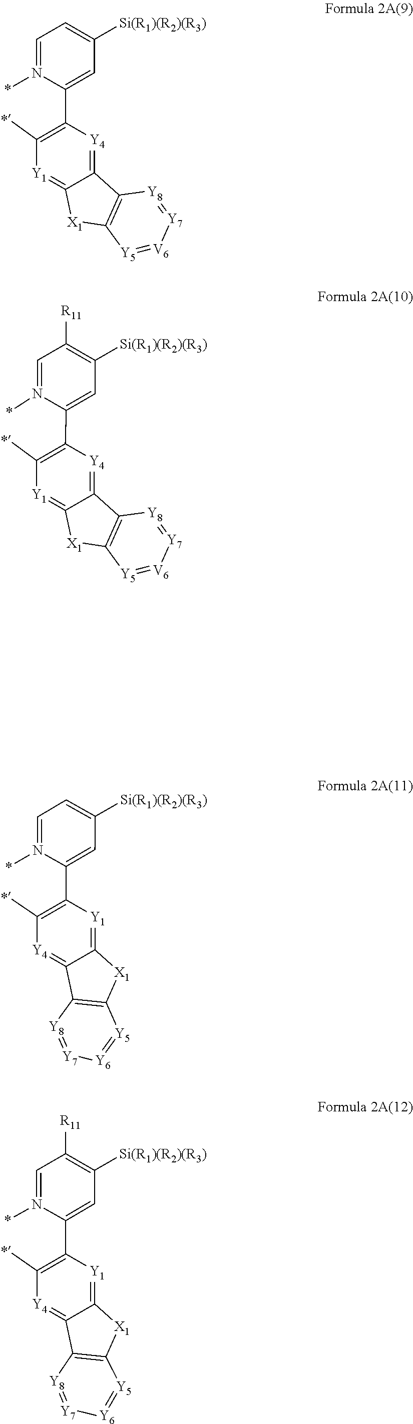 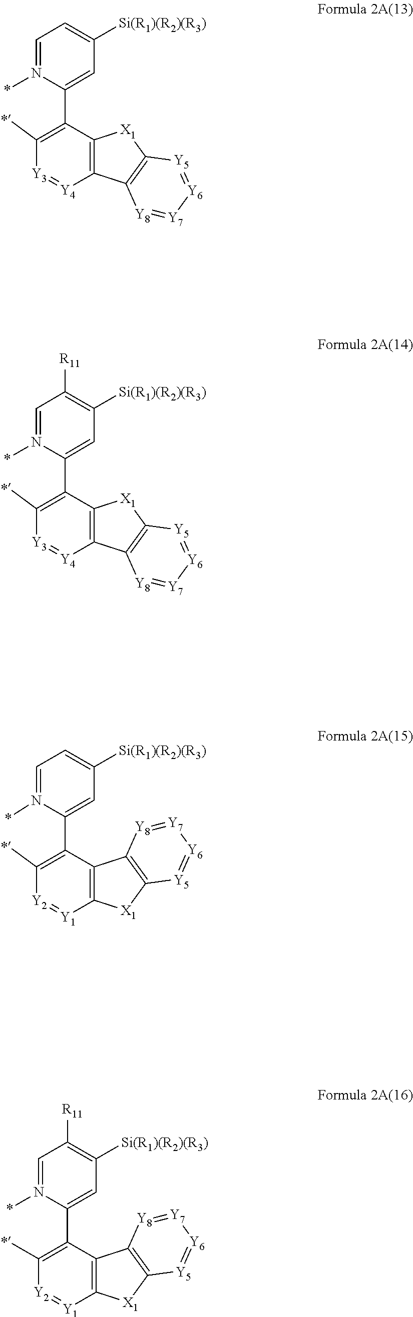 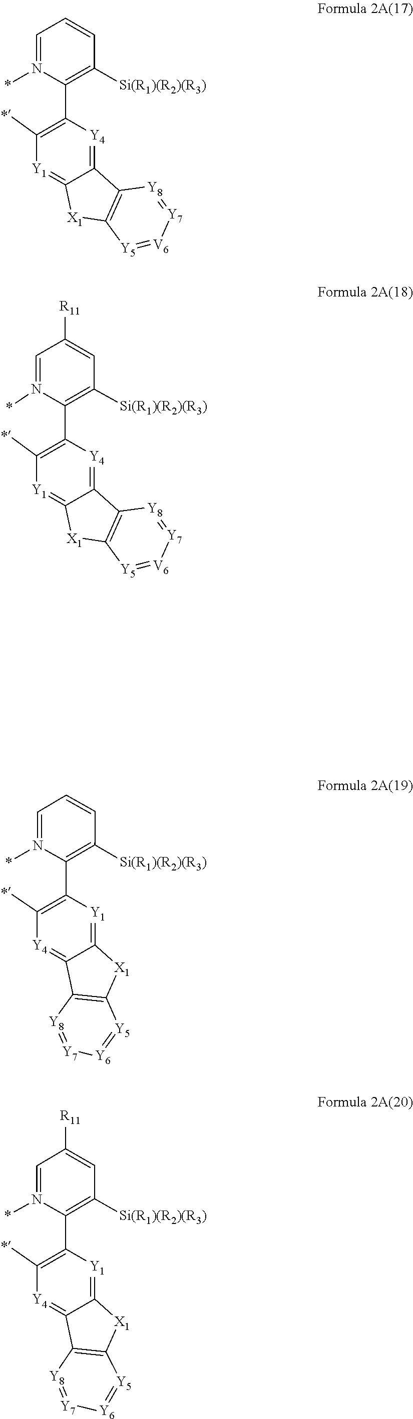 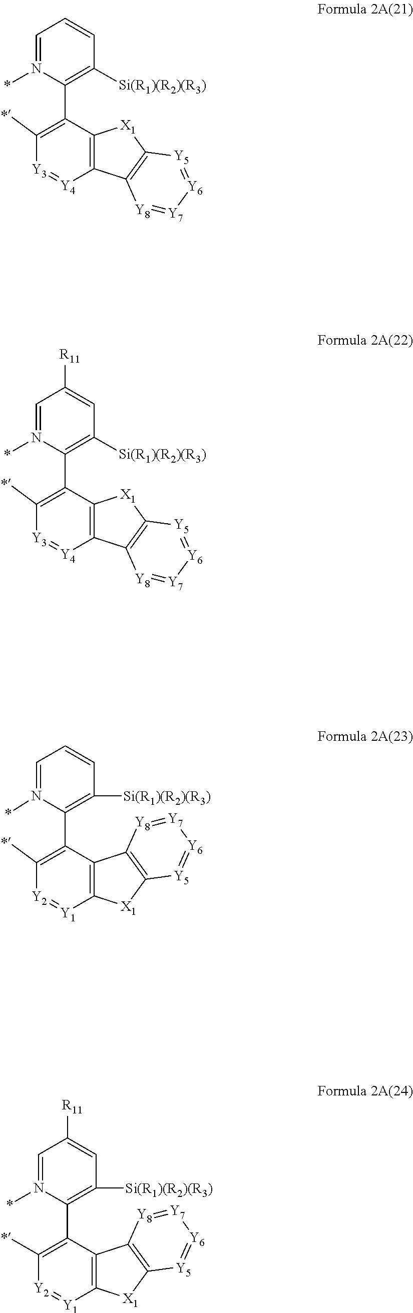 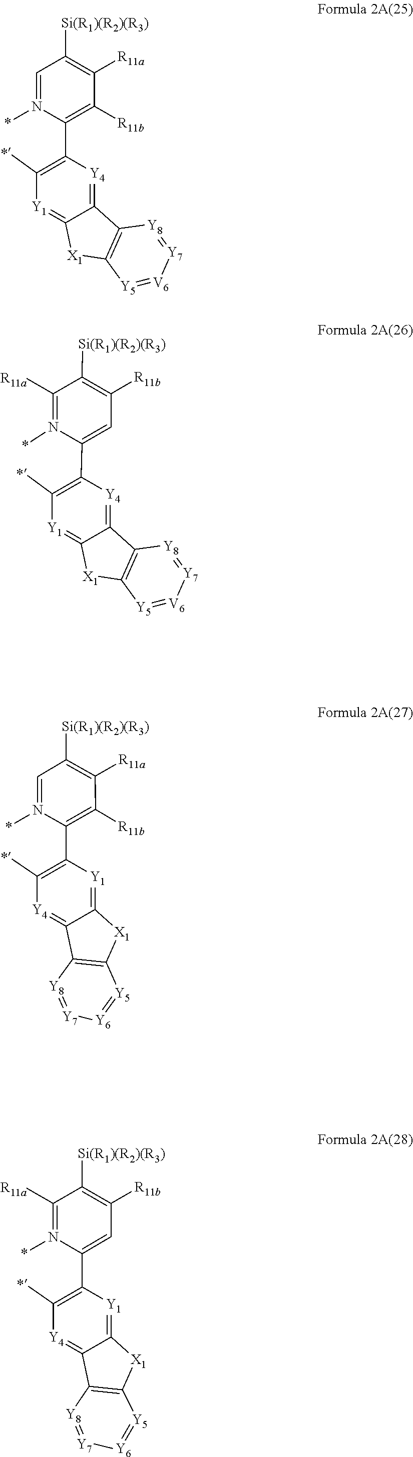 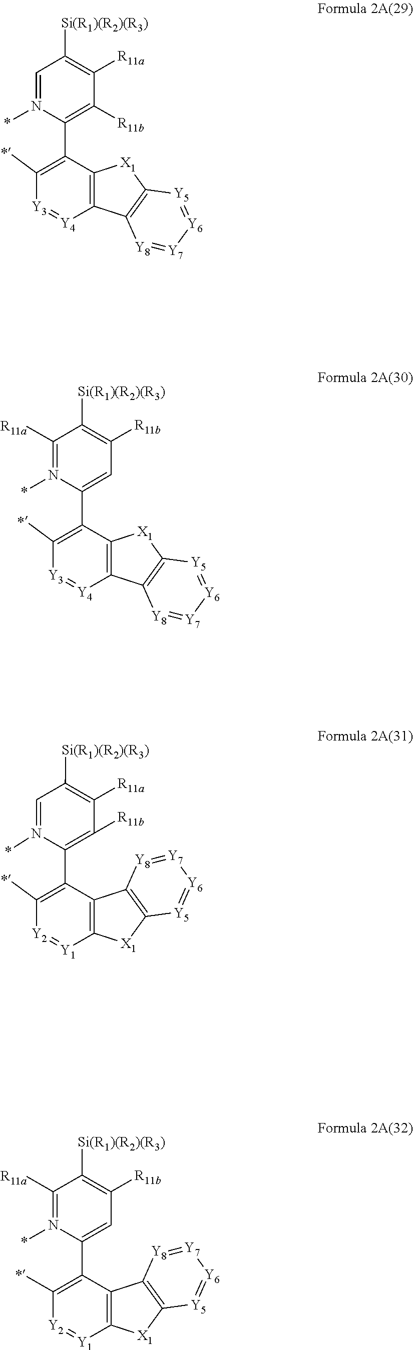 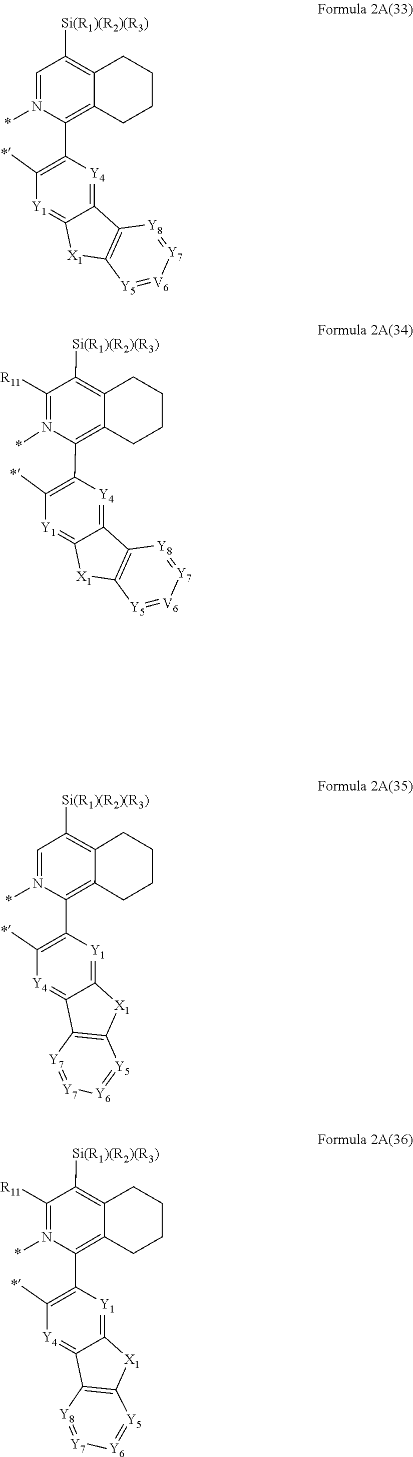 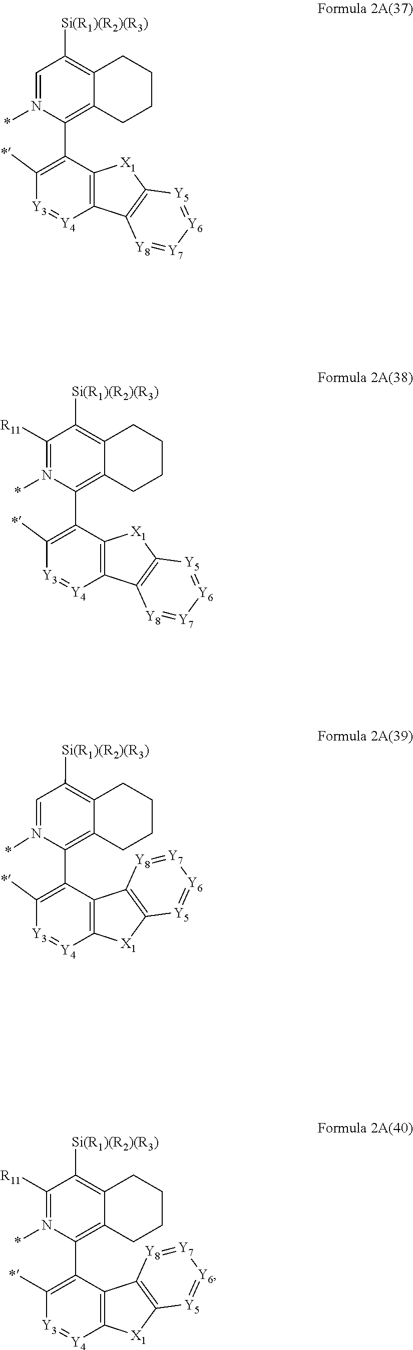
wherein, in Formulae 2A(1) and 2A(40),
R₁ to R₃, Y₁ to Y₈, and R₁₁ are the same as in claim 1,
R₁₁ₐ and R_{11b} are the same as in connection with R₁₁, provided that R₁₁, R₁₁ₐ, and R_{11b} are not hydrogen, and
* and *′ each indicate a binding site to M in Formula 1.

10. The organic light-emitting device of claim 1, wherein
L₂ in Formula 1 is selected from ligands represented by Formulae 3A to 3G: 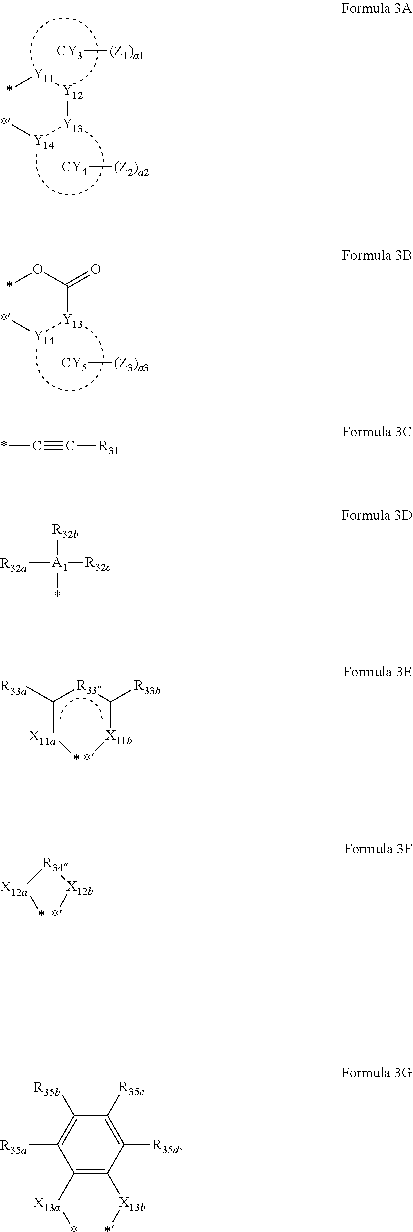
wherein, in Formulae 3A to 3G,
Y₁₁ to Y₁₆ are each independently carbon (C) or nitrogen (N), Y₁₁ and Y₁₂ are linked via a single bond or a double bond, Y₁₃ and Y₁₄ are linked via a single bond or a double bond, and Y₁₅ and Y₁₆ are linked via a single bond or a double bond,
CY₃ to CY₅ are each independently selected from a C₅-C₆₀ carbocyclic group and a C₂-C₆₀ heterocyclic group, and CY₃ and CY₄ are optionally additionally linked via an organic linking group,
a1 to a3 are each independently an integer selected from 1 to 5,
A₁ is P or As,
X₁₁ₐ, X_{11b}, X₁₂ₐ, X_{12b}, X₁₃ₐ, and X_{13b} each independently selected from N, O, N(R₃₄), P(R₃₅)(R₃₆), and As(R₃₇)(R₃₈) (provided that X₁₂ₐ, X_{12b}, X₁₃ₐ, and X₁₃b are neither N nor O),
R_{33″} and R_{34″} are each independently selected from a single bond, a double bond, a substituted or unsubstituted C₁-C₅ alkylene group, a substituted or unsubstituted C₂-C₅ alkenylene group, and a substituted or unsubstituted C₆-C₁₀ arylene group,
Z₁ to Z₃, R₃₁, R₃₂ₐ, R_{32b}, R_{32c}, R₃₃ₐ, R_{33b}, R₃₄ to R₃₈, R₃₅ₐ, R_{35b}, R_{35c}, and R_{35d} are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), and —P(═O)(Q₈)(Q₉),
* and *′ each indicate a binding site to M in Formula 1, and
at least one substituent selected from a substituent(s) of the substituted C₁-C₅ alkylene group, the substituted C₂-C₅ alkenylene group, the substituted C₆-C₁₀ arylene group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group is selected from:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and
—N(Q₃₁)(Q₃₂), —Si(Q₃₃)(Q₃₄)(Q₃₅), —B(Q₃₆)(Q₃₇), and —P(═O)(Q₃₈)(Q₃₉),
wherein Q₁ to Q₉, Q₁₁ to Q₁₉, C₂₁ to Q₂₉, and to Q₃₉ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

11. The organic light-emitting device of claim 1, wherein
L₂ in Formula 1 is selected from ligands represented by Formulae 3-1(1) to 3-1(59) and 3-111: 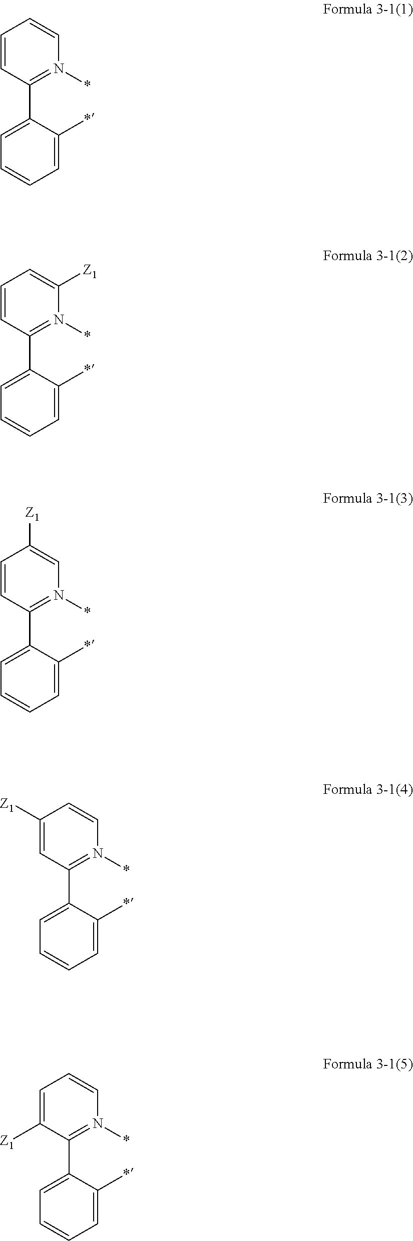 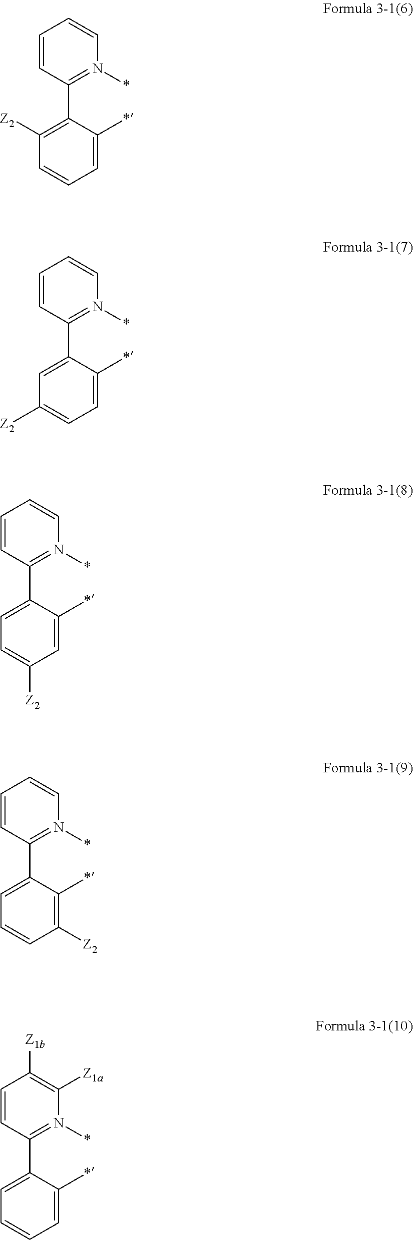 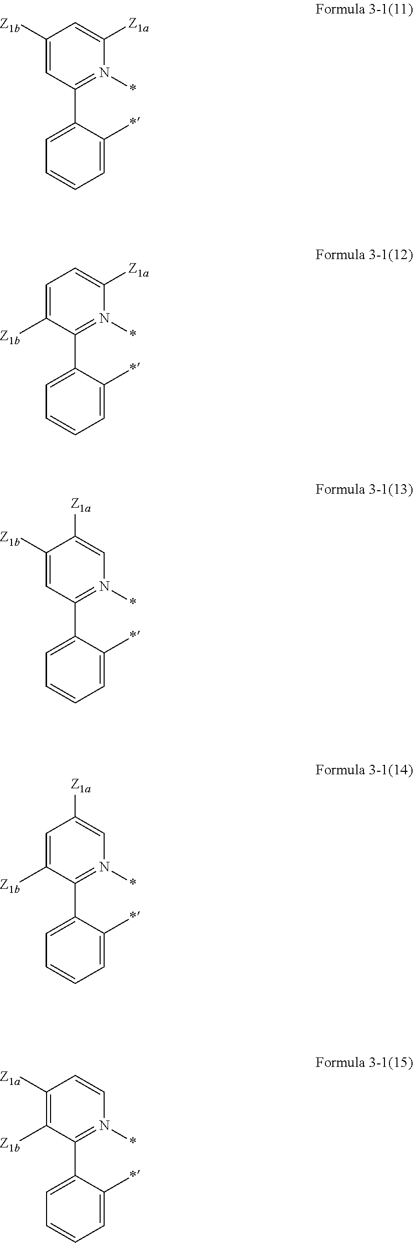 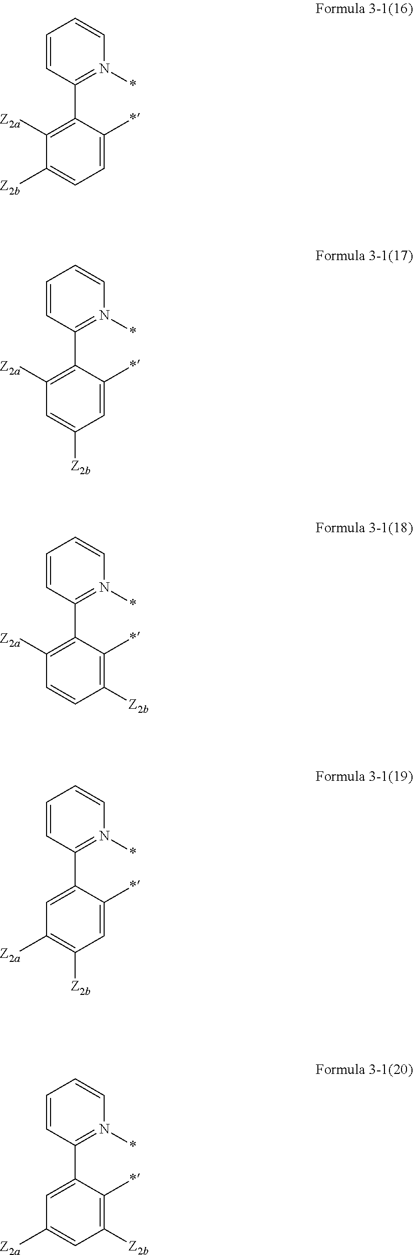 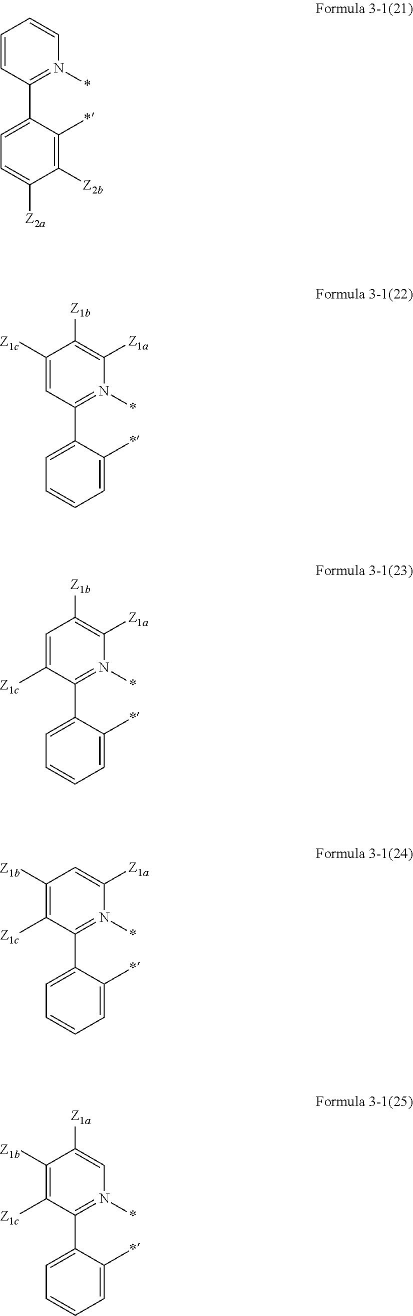 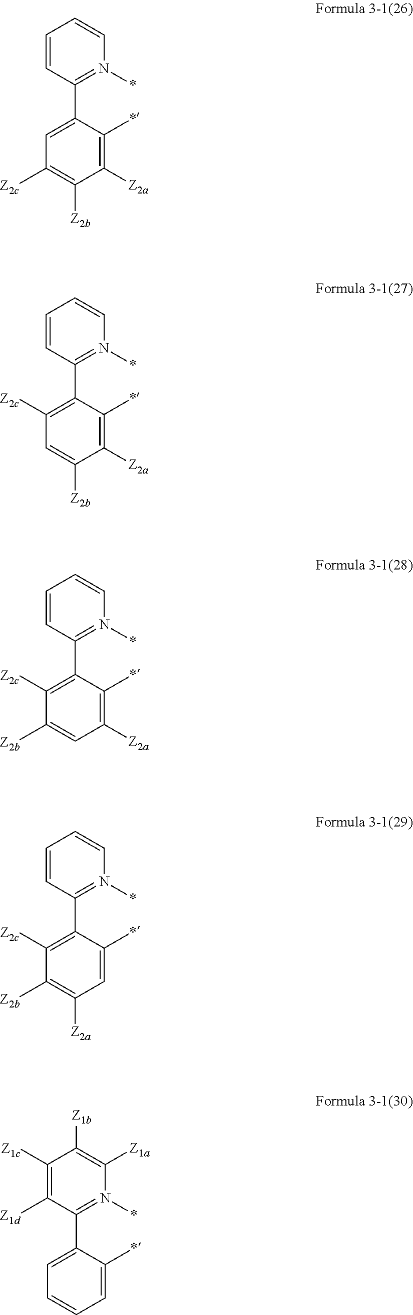 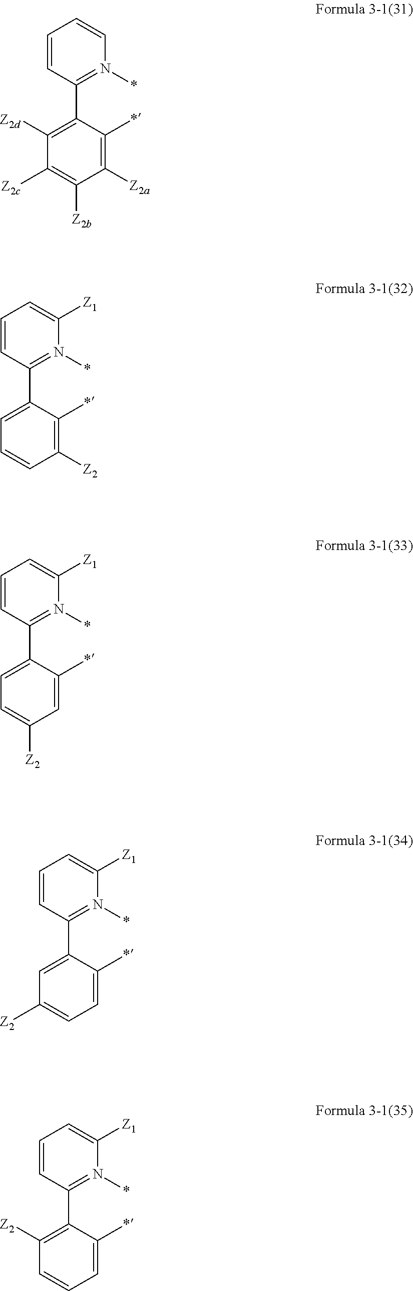 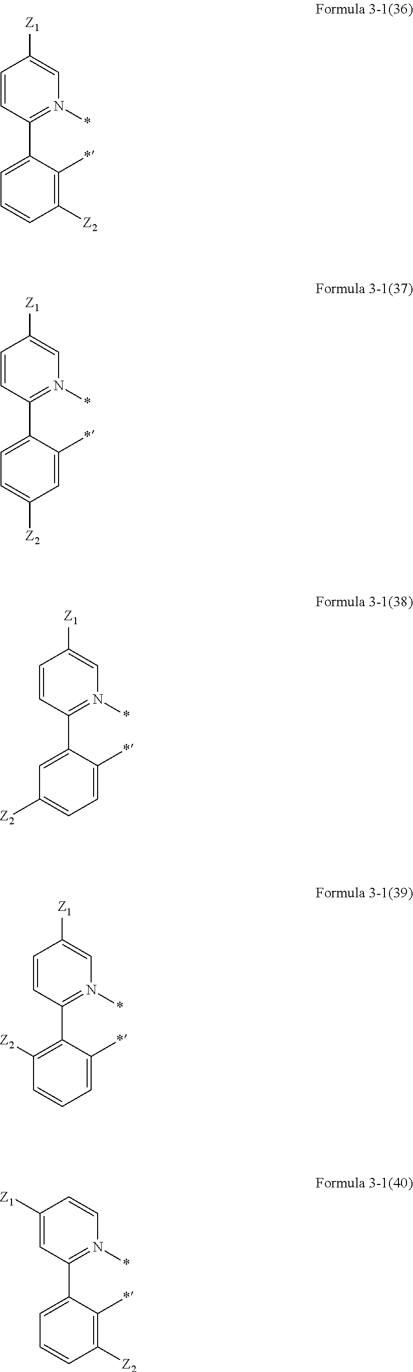 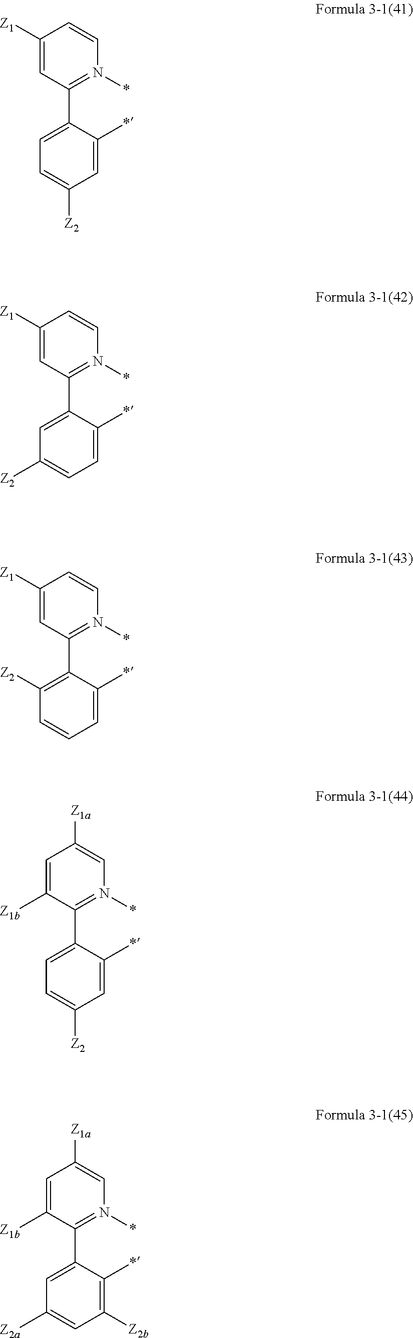 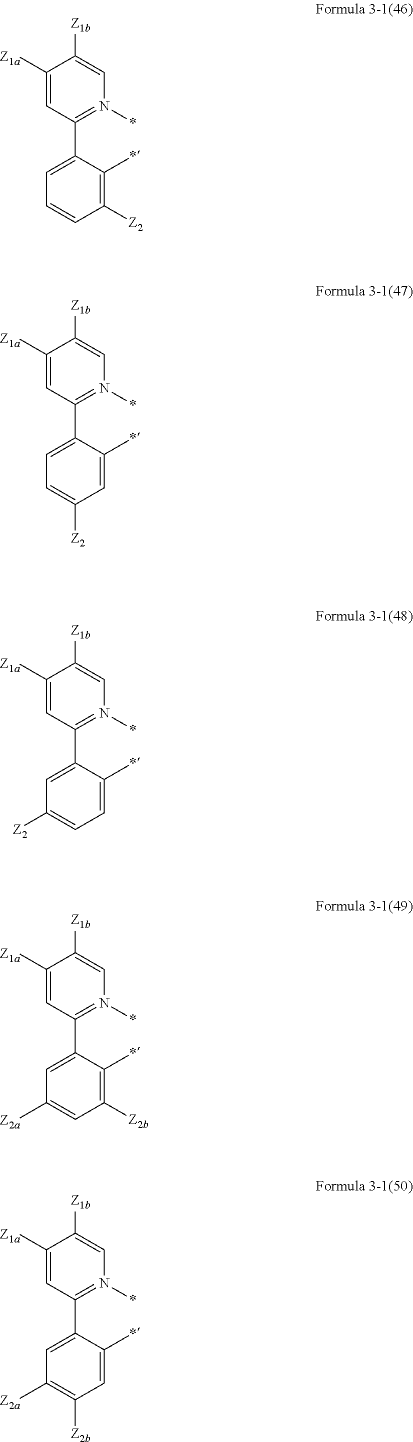 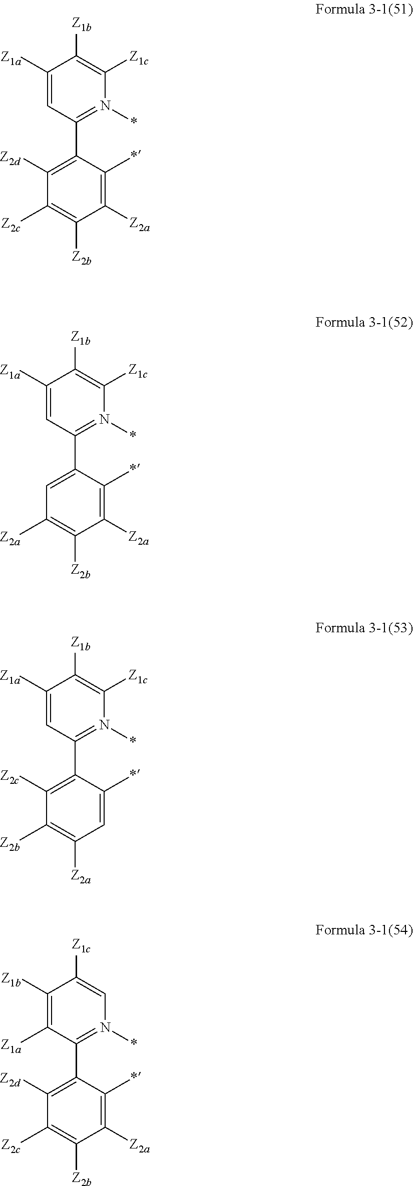 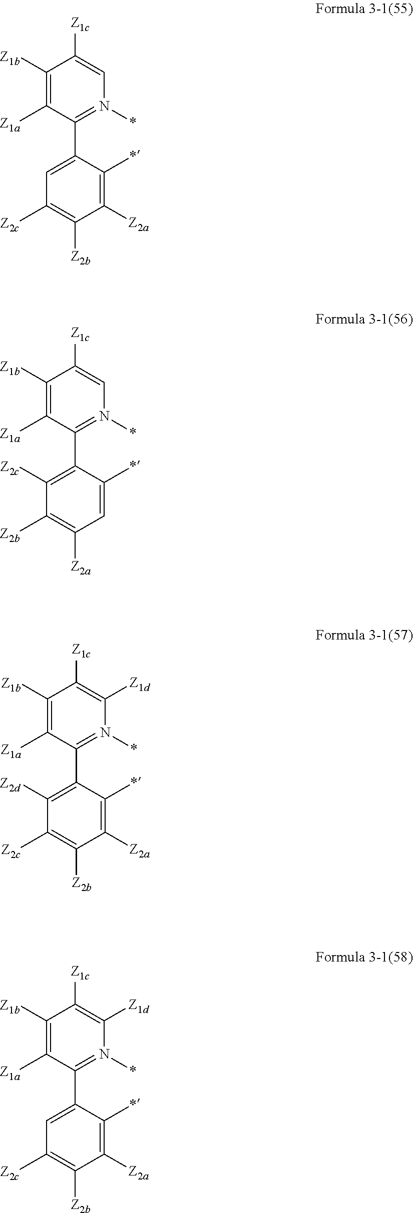 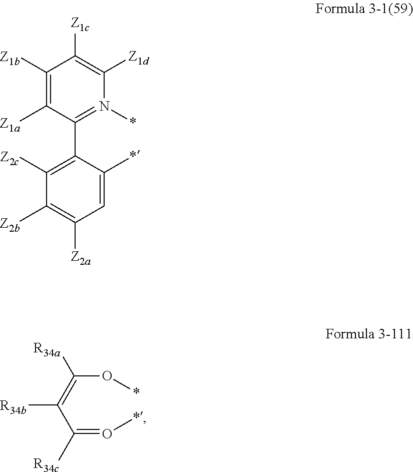
wherein, in Formulae 3-1(1) to 3-1(59) and 3-111,
Z₁, Z₂, Z₁ₐ, Z_{1b}, Z_{1c}, Z_{1d}, Z₂ₐ, Z_{2b}, Z_{2c}, Z₂d, R₃₄ₐ, R_{34b}, and R_{34c} are each independently selected from deuterium, —F, a cyano group, a nitro group, —SF₅, —CH₃, —CD₃, —CD₂H, —CDH₂, —CF₃, —CF₂H, —CFH₂, —Si(Q₃)(Q₄)(Q₅), groups represented by Formulae 9-1 to 9-17, and groups represented by Formulae 10-1 to 10-30,
Q₃ to Q₅ are each independently selected from:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;
an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group; and
an n-propyl group, an iso-propyl group, an n-butyl group, an iso-butyl group, a sec-butyl group, a tert-butyl group, an n-pentyl group, an isopentyl group, a sec-pentyl group, a tert-pentyl group, a phenyl group, and a naphthyl group, each substituted with at least one selected from deuterium and a C₁-C₁₀ alkyl group: 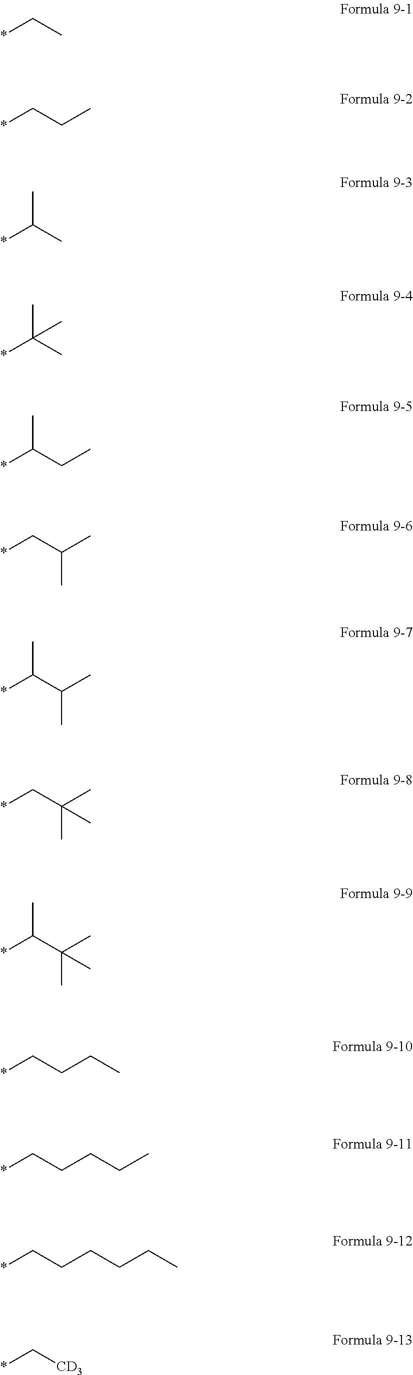 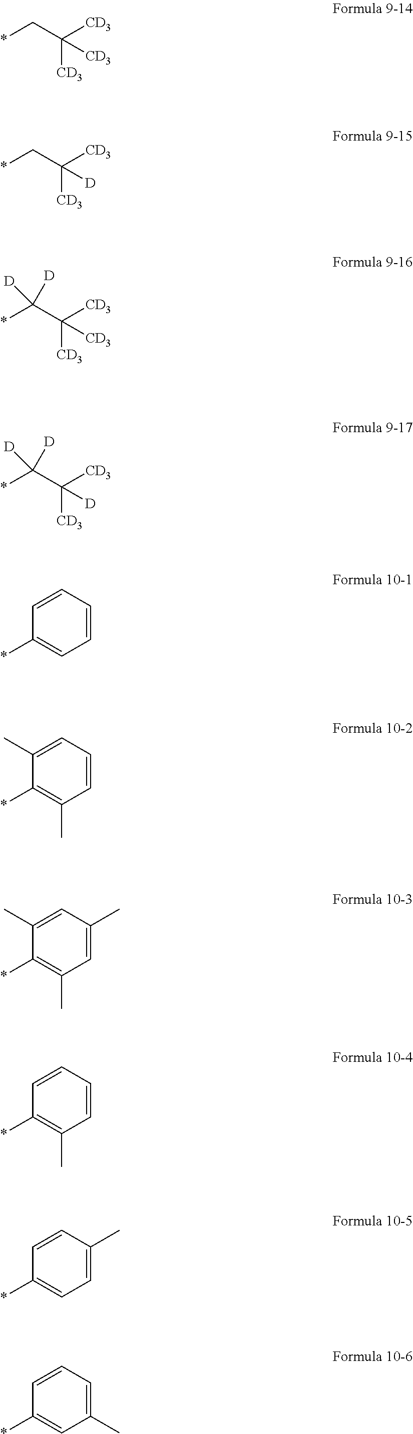 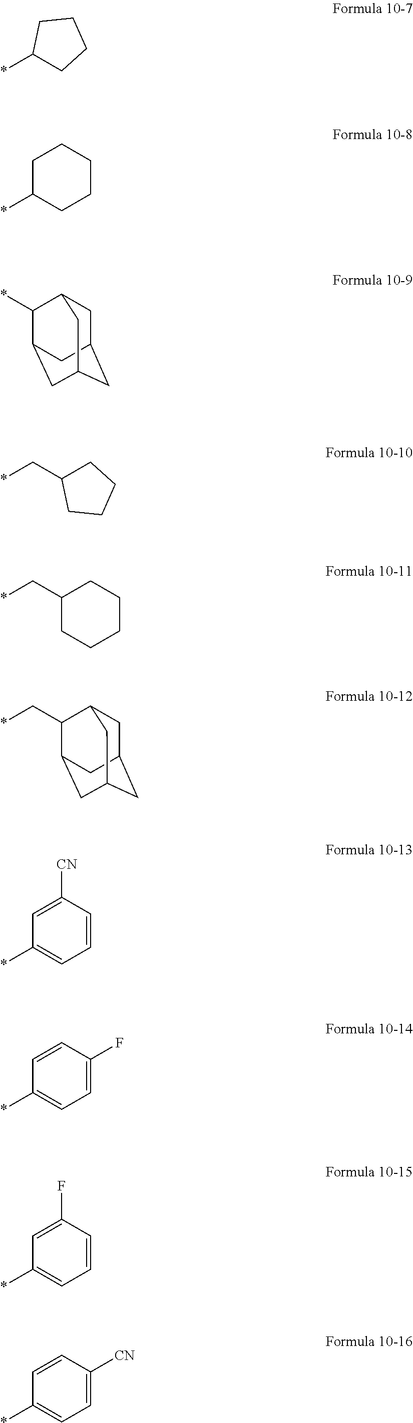 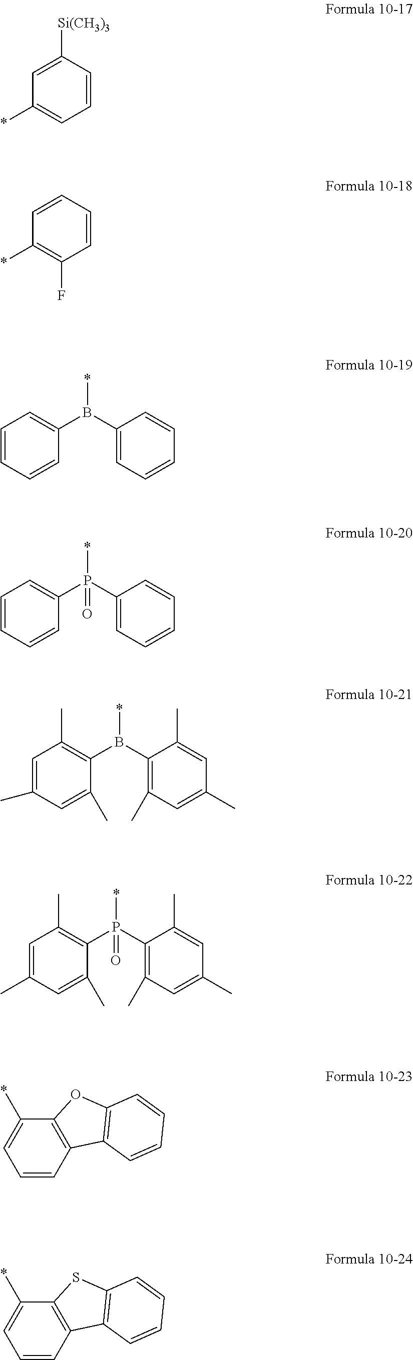 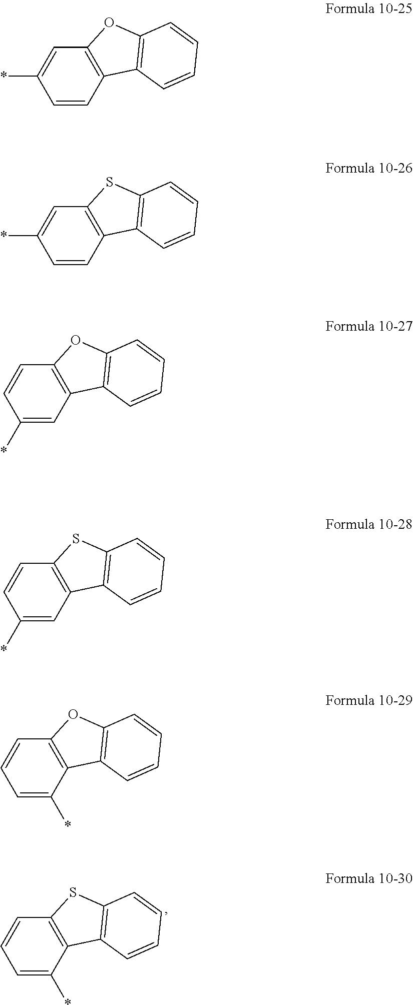 [SMILES: *c1ccc2c(c1)oc1ccccc12 ; *c1ccc2c(c1)sc1ccccc12 ; *c1ccc2oc3ccccc3c2c1 ; *c1ccc2sc3ccccc3c2c1 ; *c1cccc2oc3ccccc3c12 ; *c1cccc2sc3ccccc3c12]
wherein * in Formulae 9-1 to 9-17 and 10-1 to 10-30 indicates a binding site to a neighboring atom.

12. The organic light-emitting device of claim 1, wherein
the organometallic compound is one selected from following Compounds:

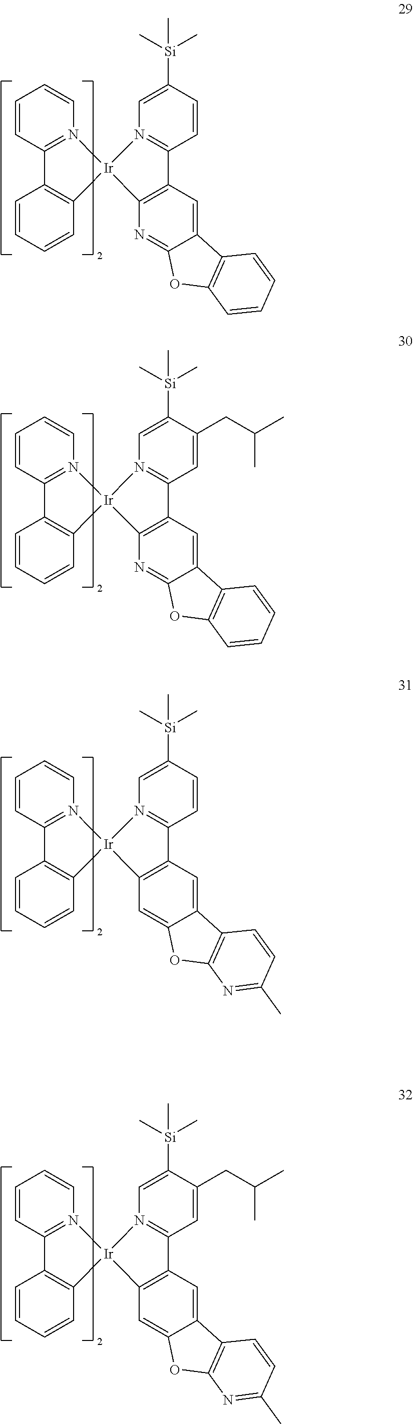

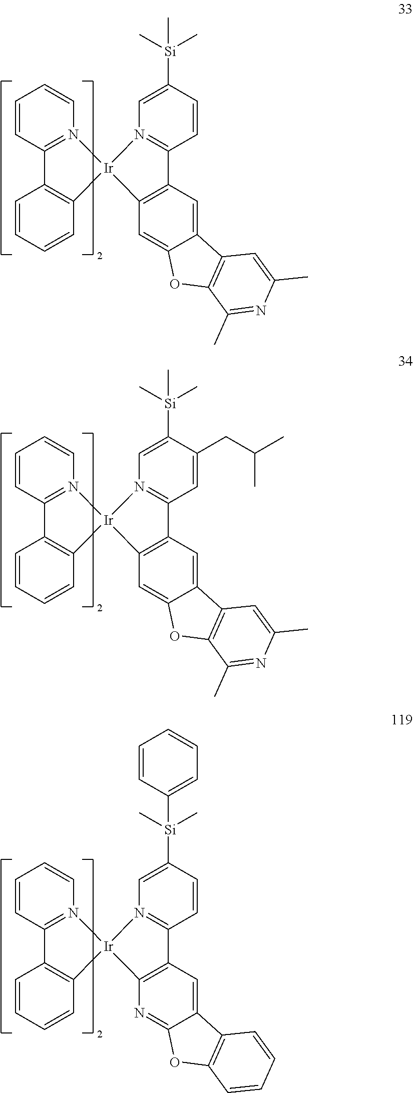

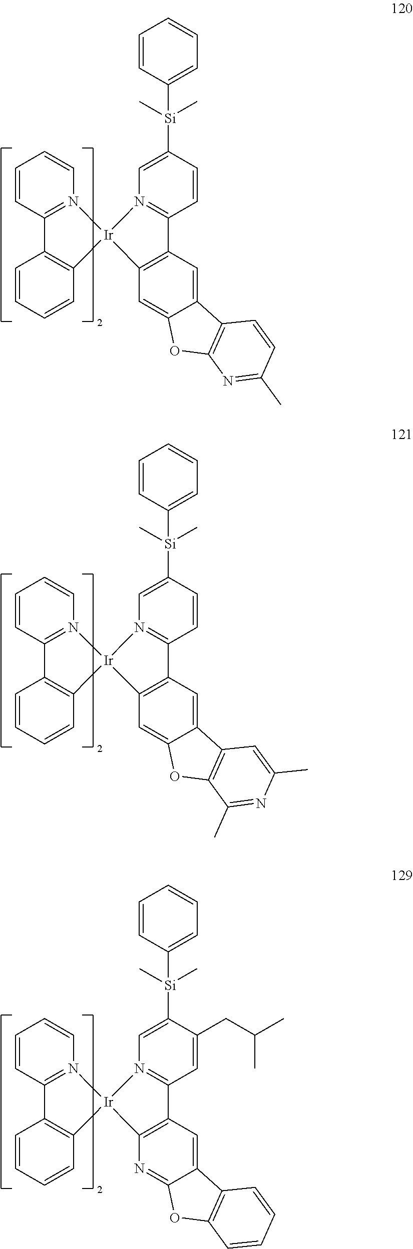

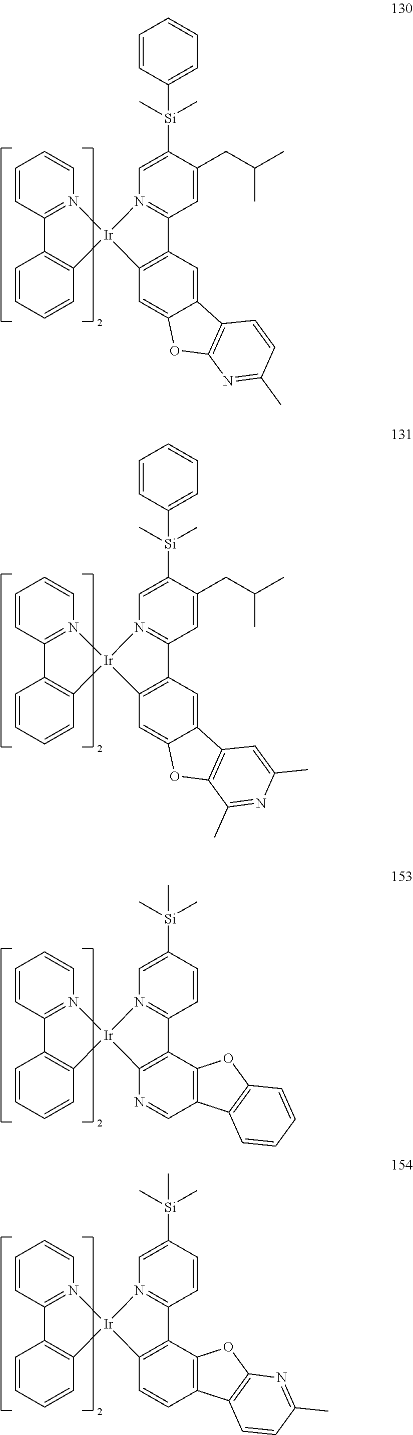

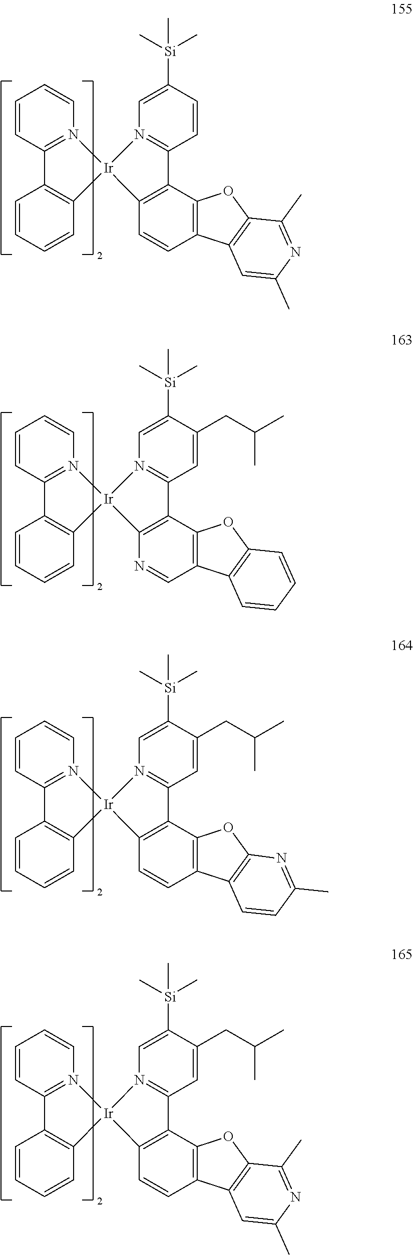

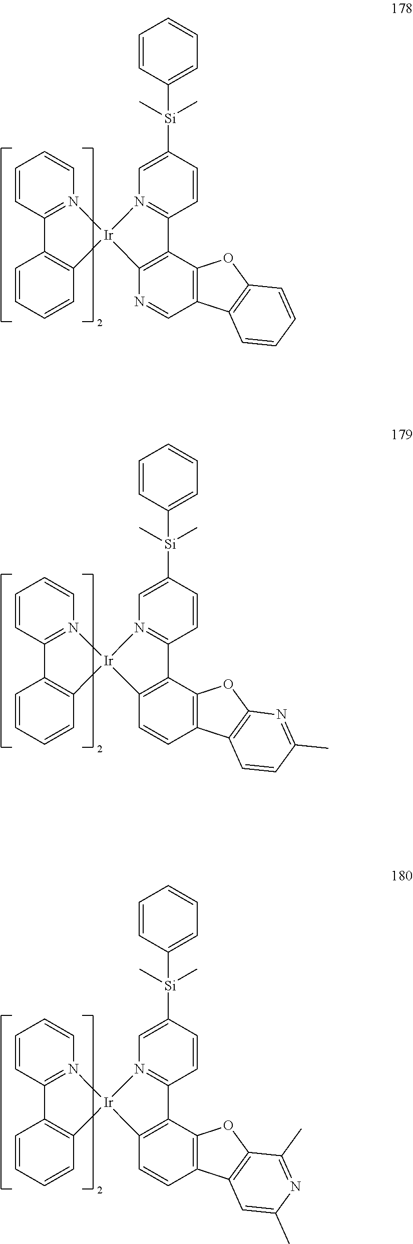

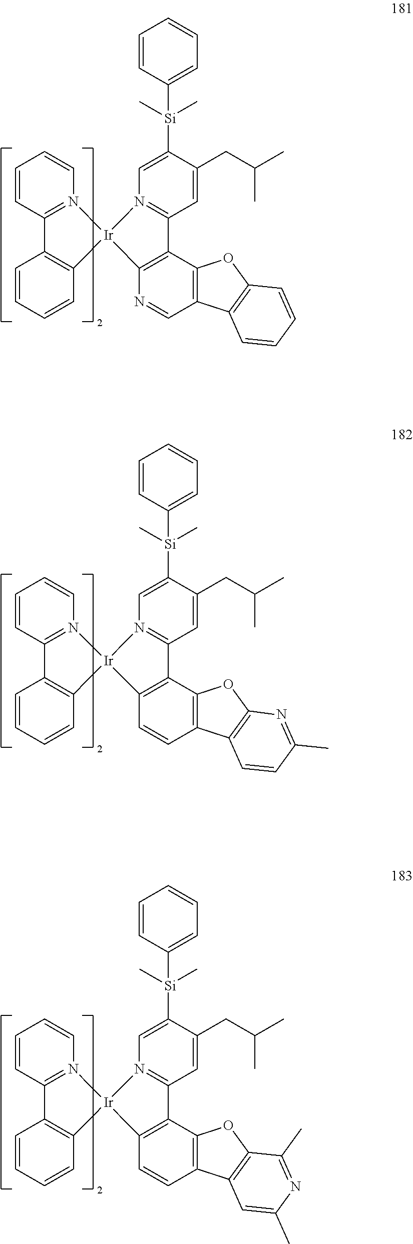

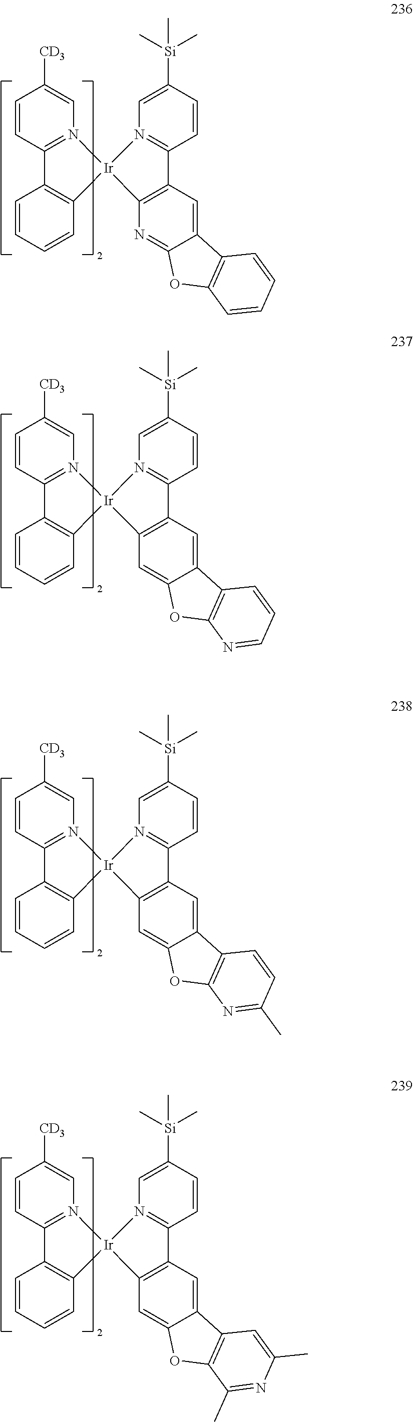

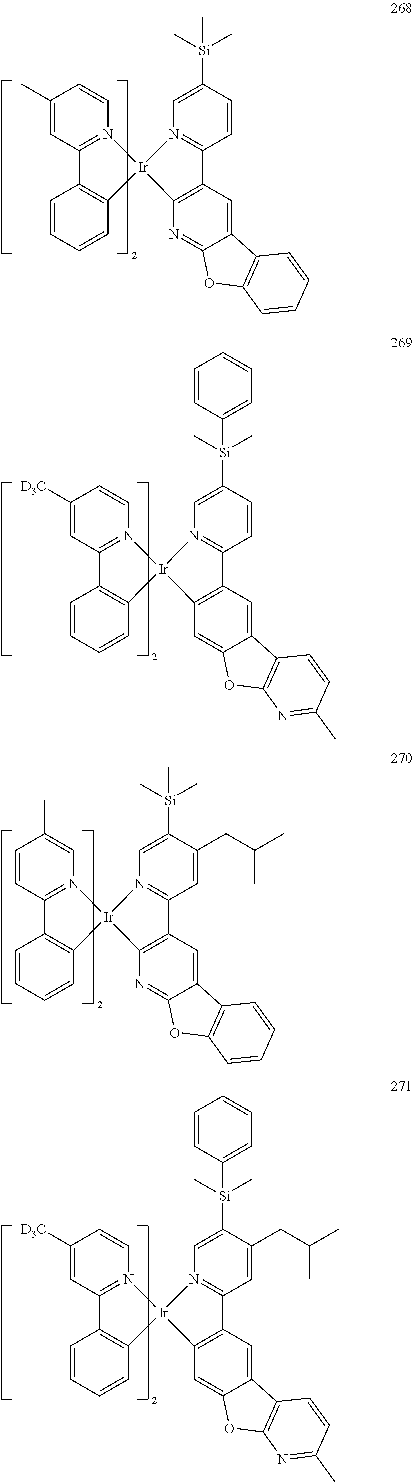

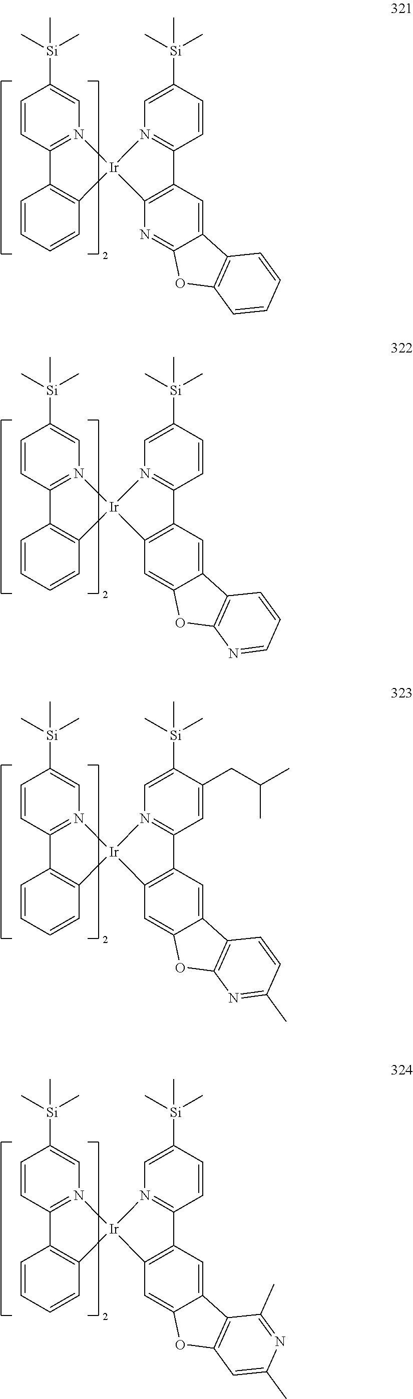

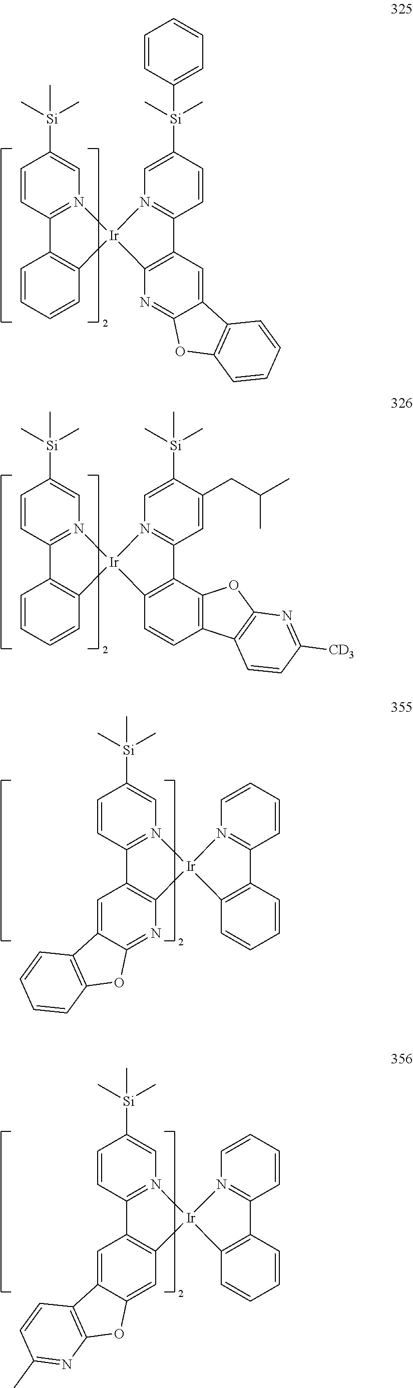

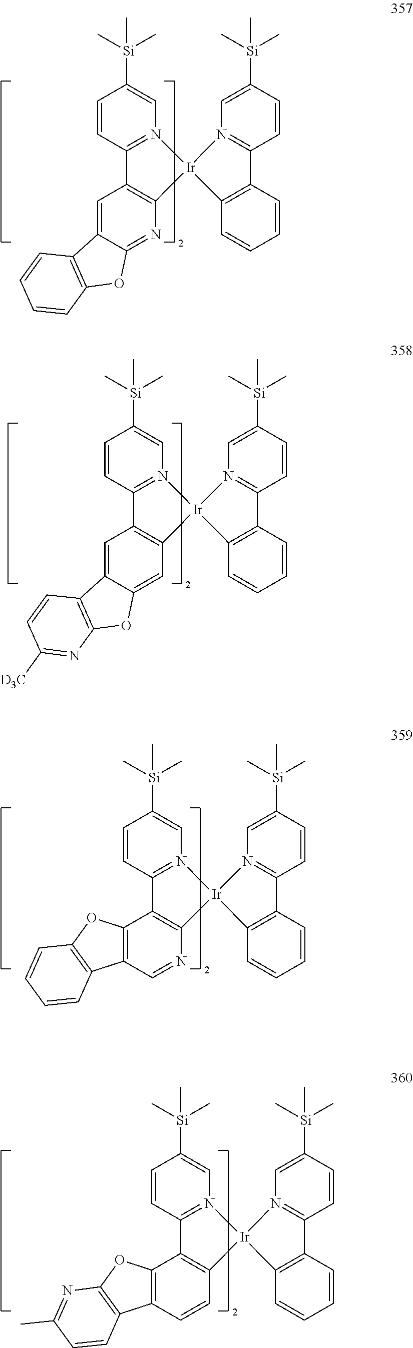

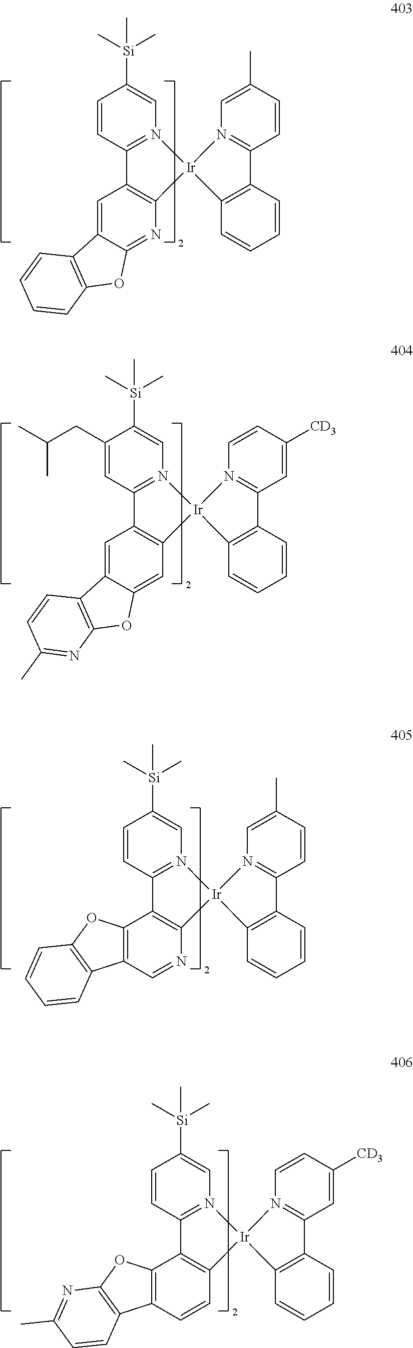

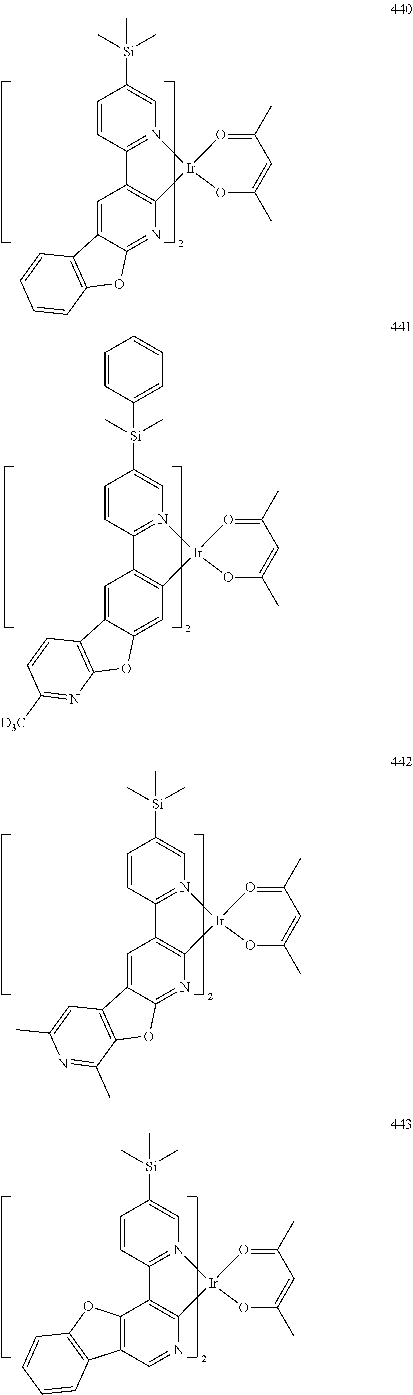

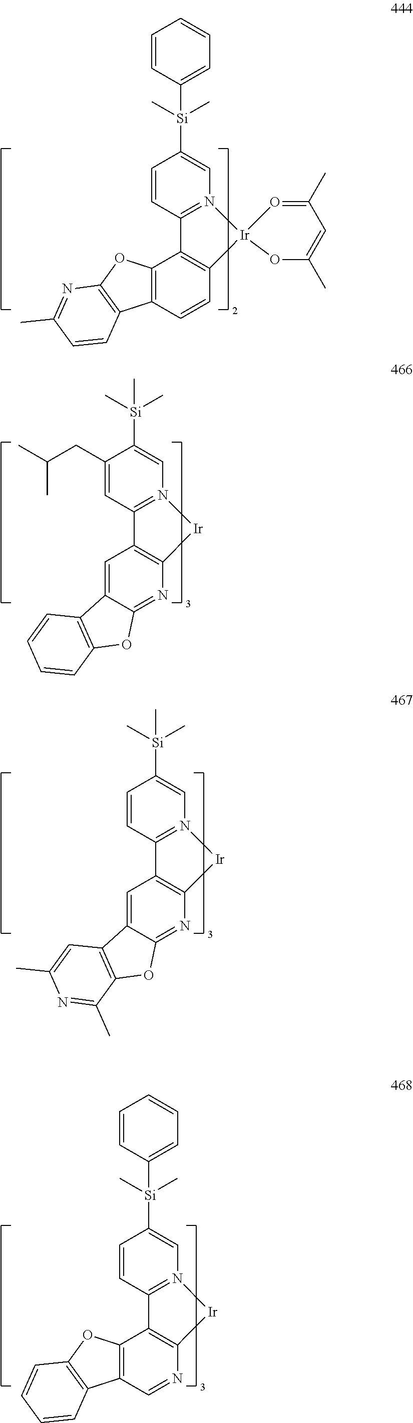

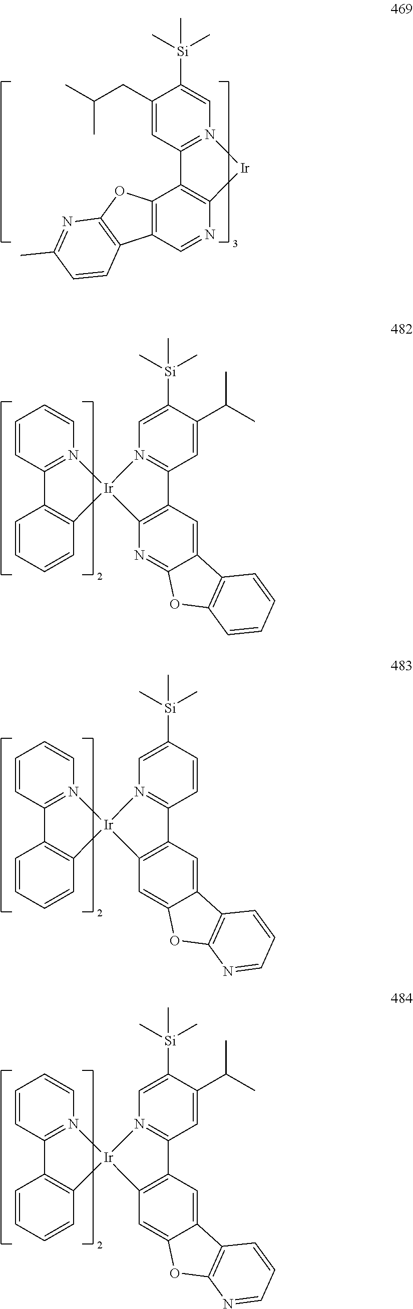

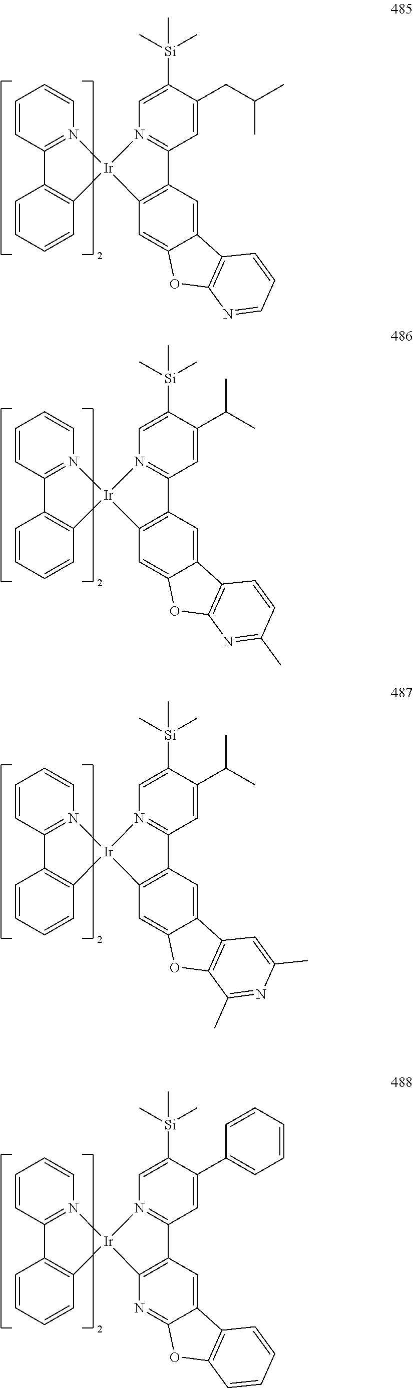

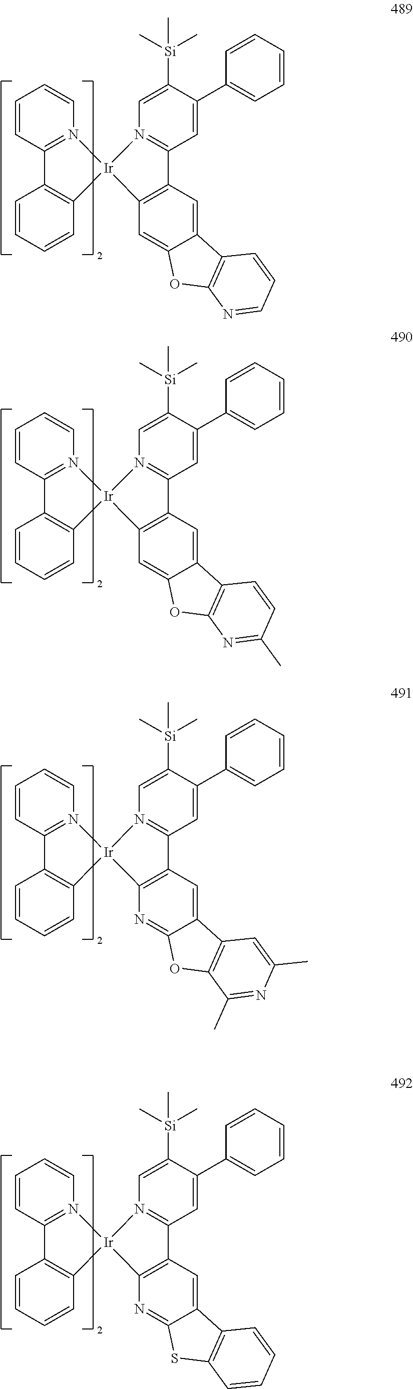

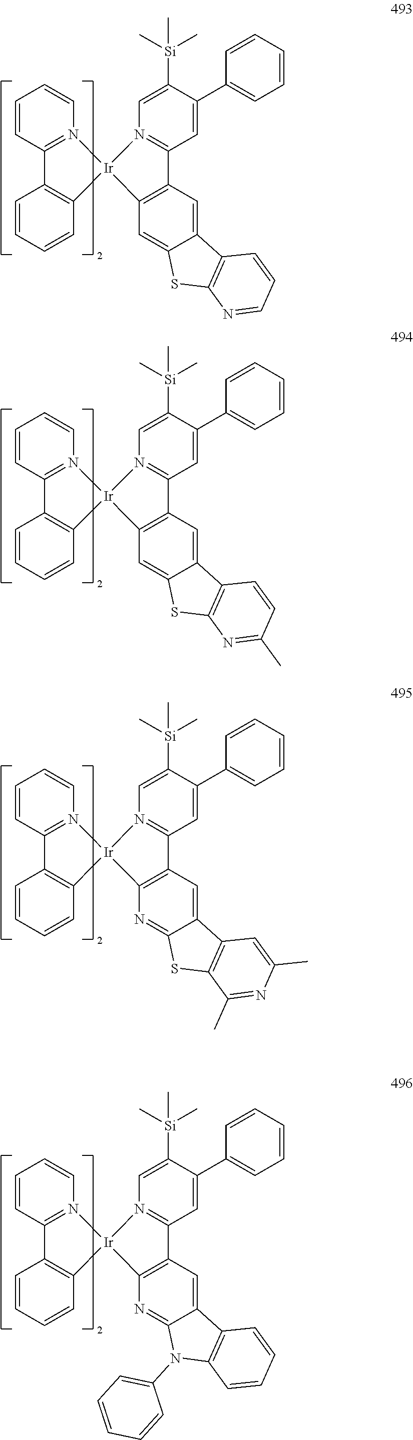

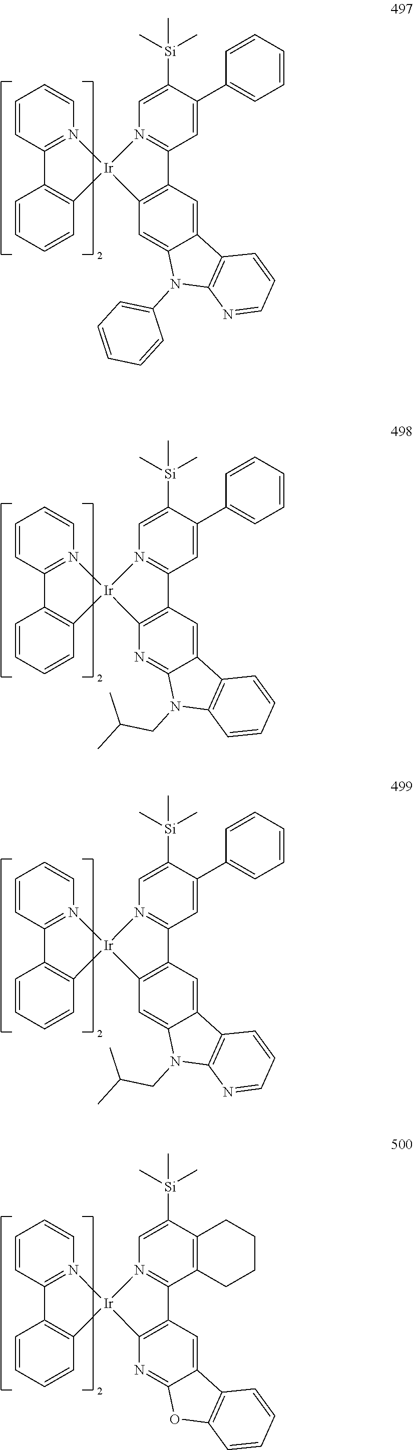

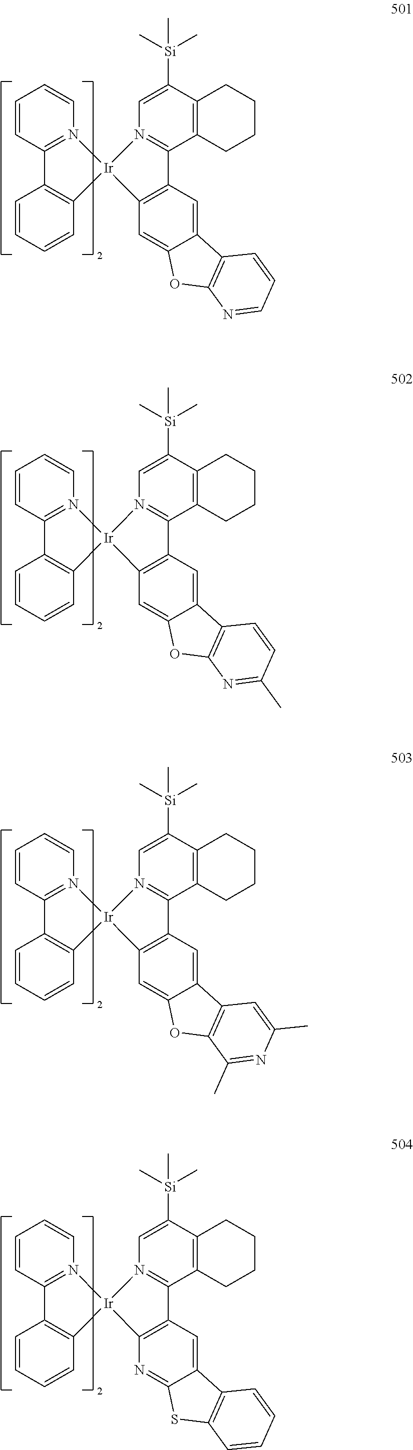

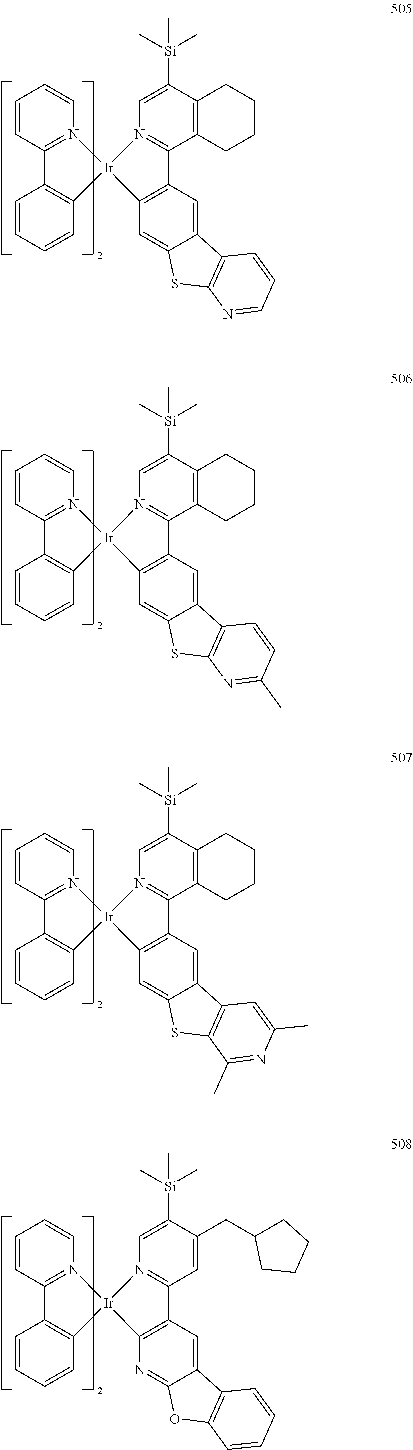

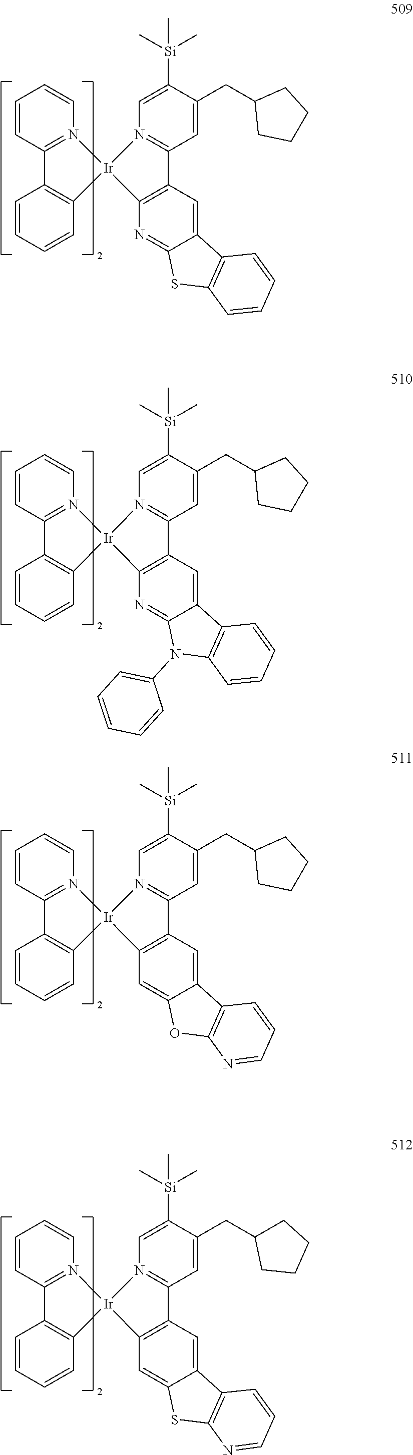

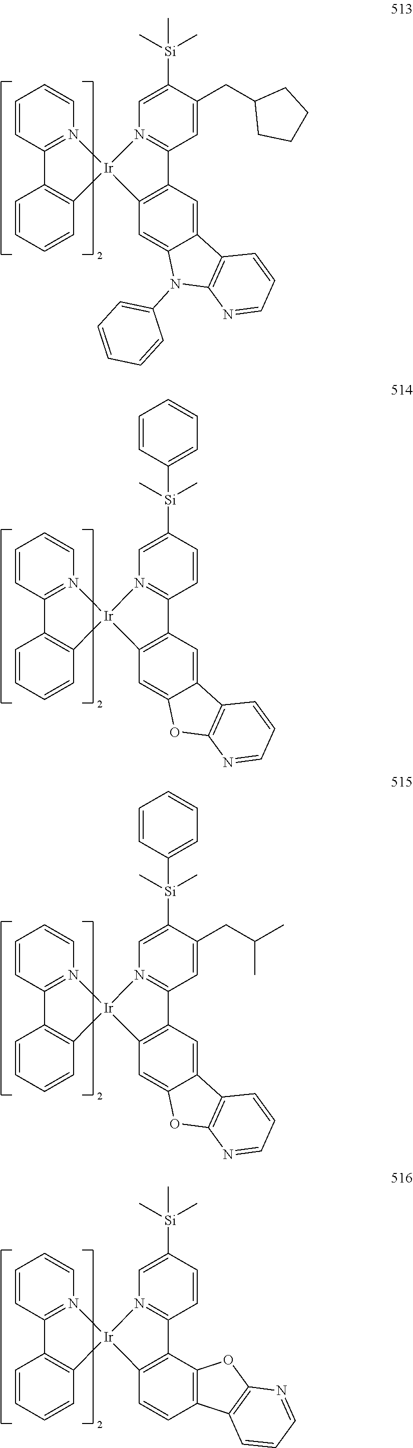

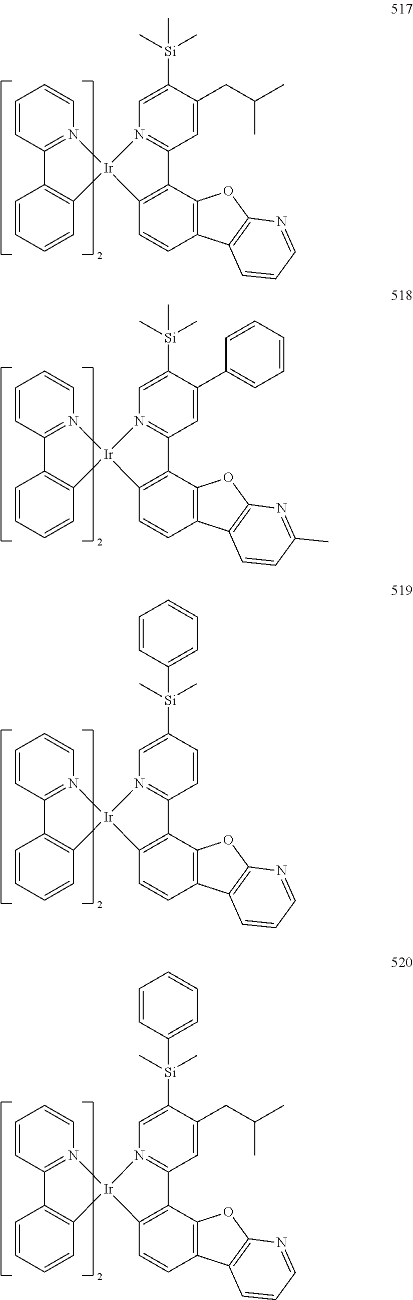

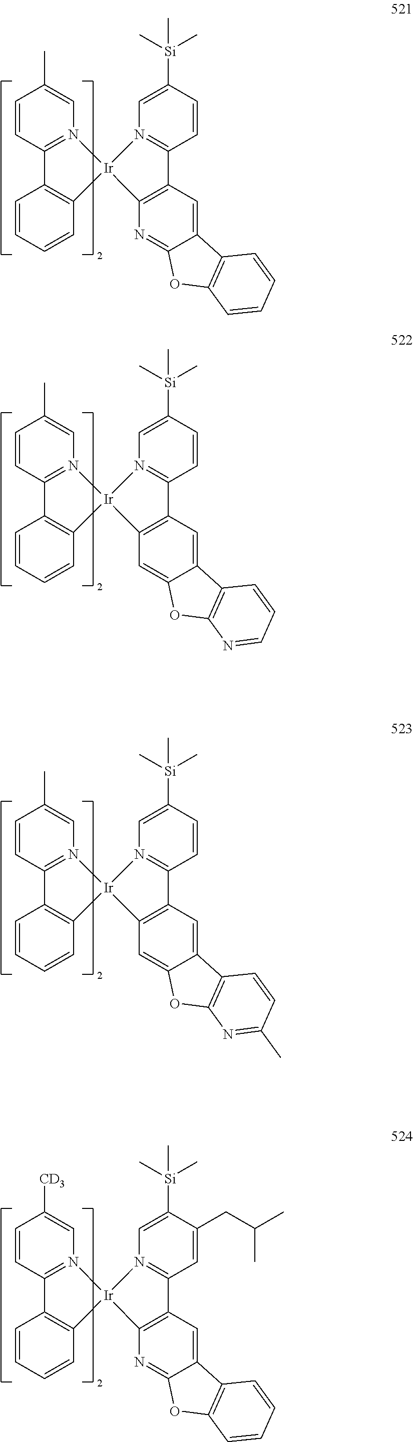

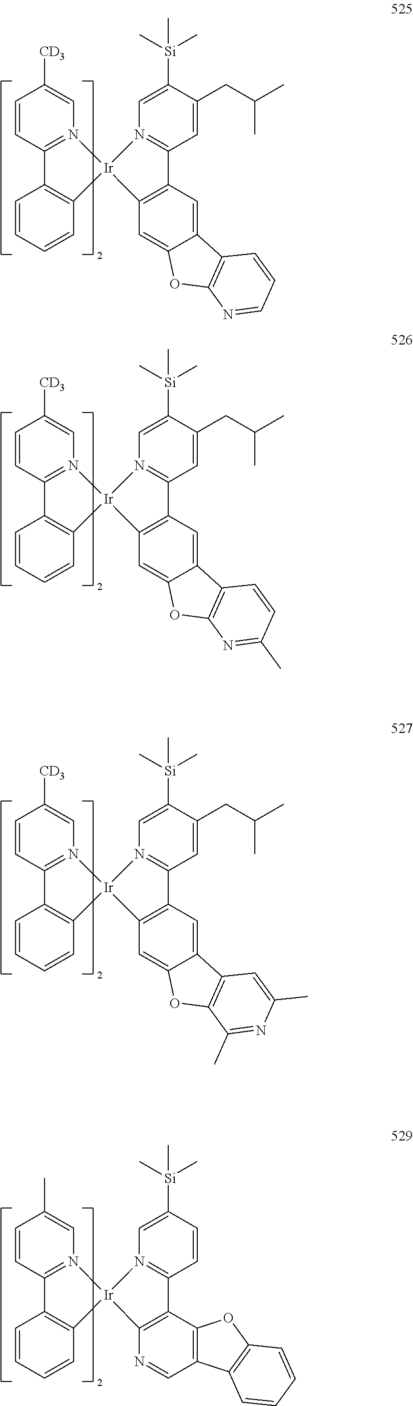

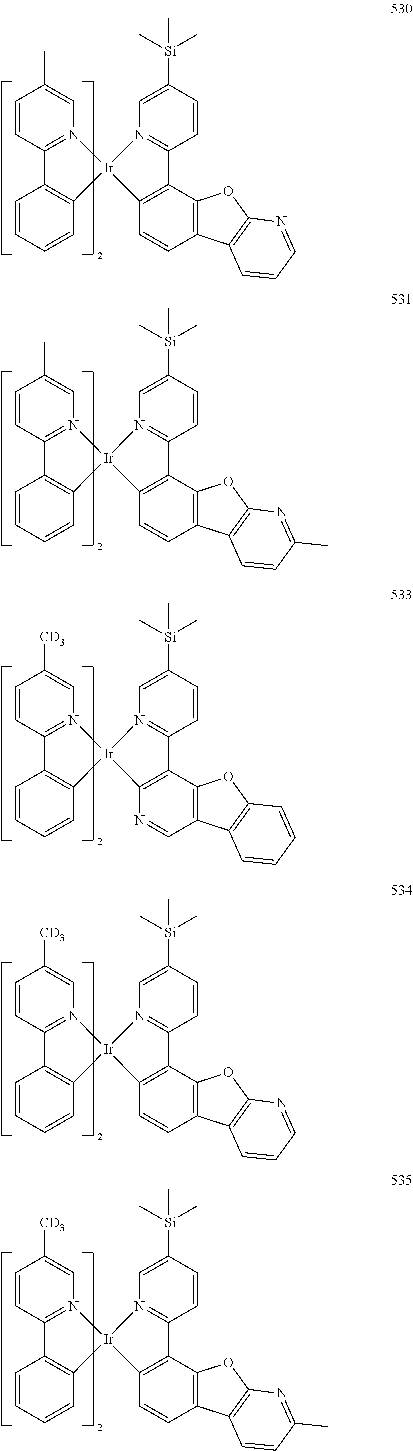

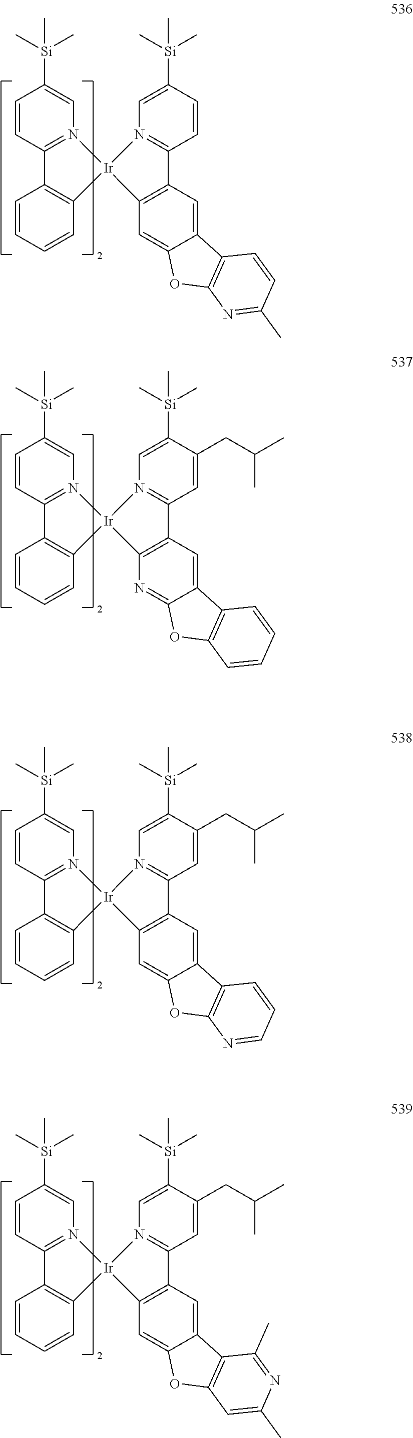

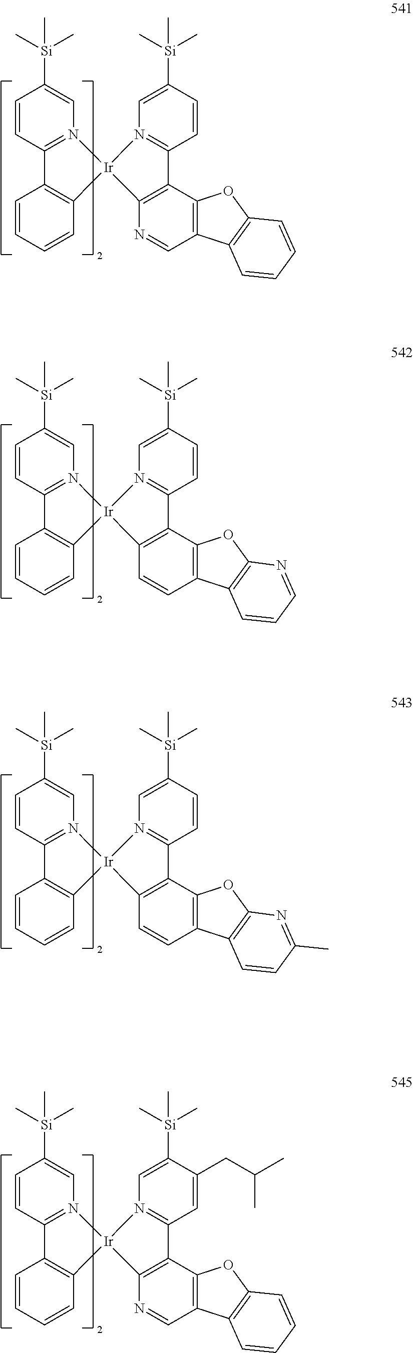

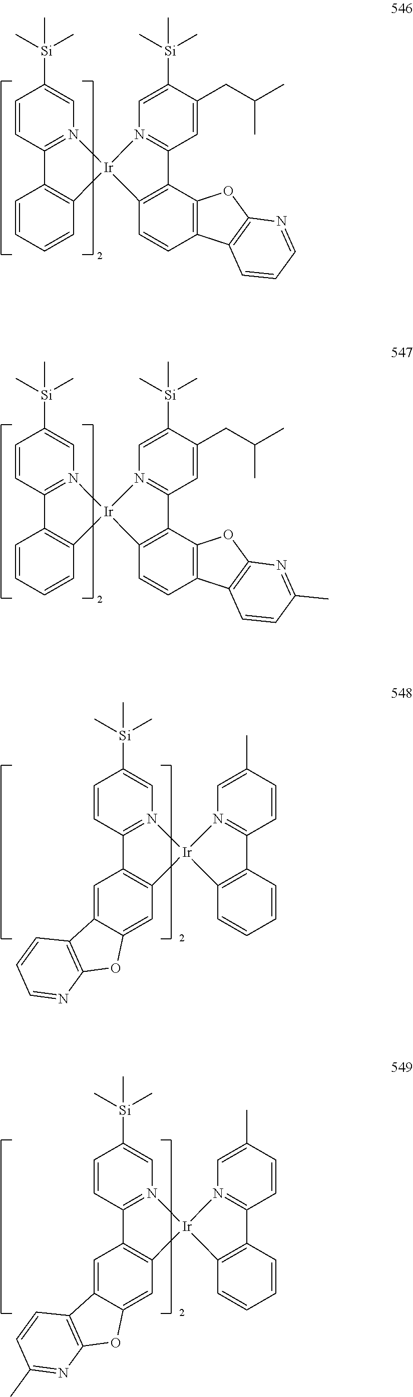

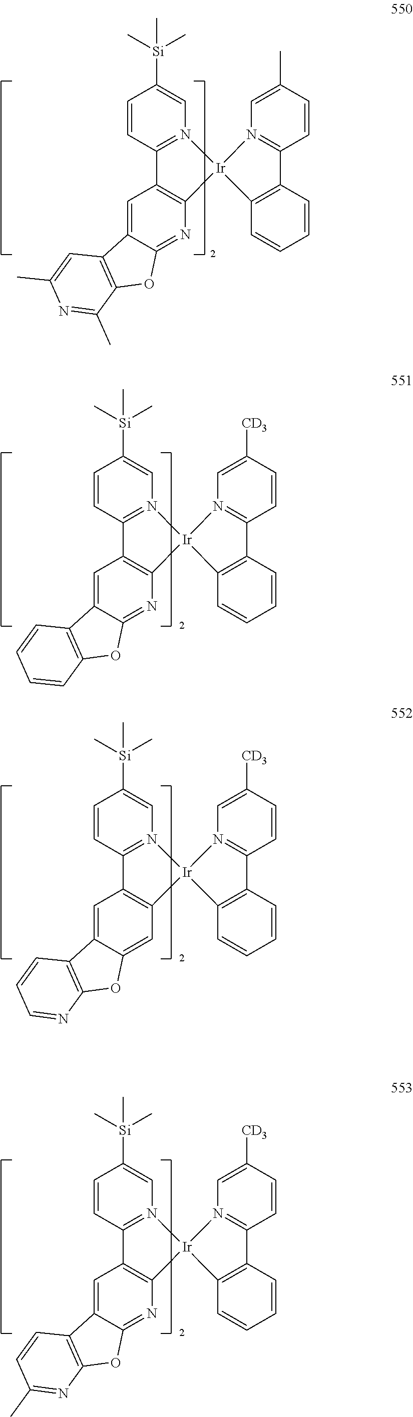

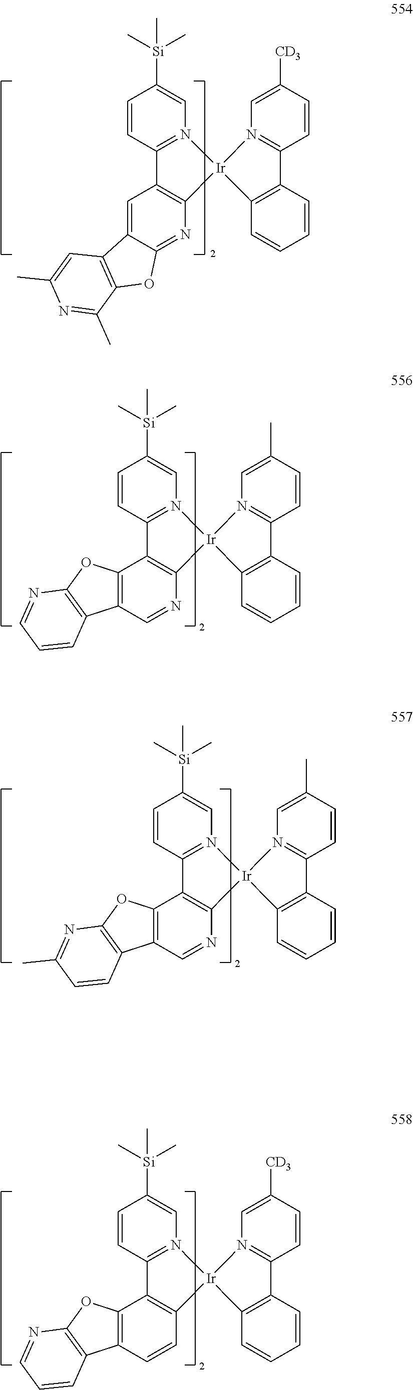

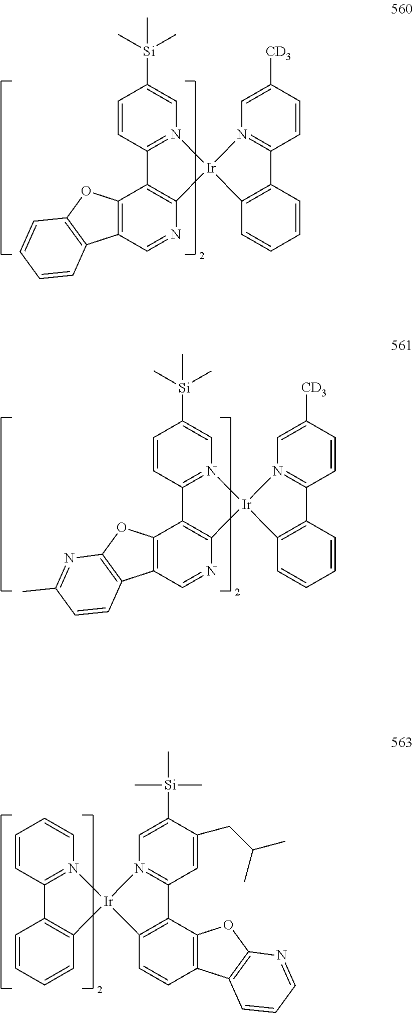

13. The organic light-emitting device of claim 1, wherein
L₅₁ to L₅₃, L₆₁, L₆₂, and to L₉₅ in Formulae 51, 61, 61B, and 91 are each independently selected from:
a single bond, a cyclopentylene group, a cyclohexylene group, a cyclopentenylene group, a cyclohexenylene group, a cycloheptenylene group, a phenylene group, a pentalenylene group, an indenylene group, a naphthylene group, an azulenylene group, a heptalenylene group, an indacenylene group, an acenaphthylene group, a benzoindenylene group, a fluorenylene group, a spiro-bifluorenylene group, a benzofluorenylene group, a dibenzofluorenylene group, a phenalenylene group, a phenanthrenylene group, an anthracenylene group, a fluoranthrenylene group, a triphenylenylene group, a pyrenylene group, a chrysenylene group, a naphthacenylene group, a picenylene group, a perylenylene group, a pentaphenylene group, a hexacenylene group, an imidazolylene group, a pyrazolylene group, a pyridinylene group, a pyrazinylene group, a pyrimidinylene group, a pyridazinylene group, a triazinylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzoxazolylene group, a benzimidazolylene group, a thiazolylene group, an isothiazolylene group, a benzothiazolylene group, an isoxazolylene group, an oxazolylene group, a triazolylene group, a tetrazolylene group, an oxadiazolylene group, an imidazopyrimidinylene group, an imidazopyridinylene group, a pyrrolylene group, a furanylene group, a thiophenylene group, a silolylene group, an isoindolylene group, an indolylene group, a benzofuranylene group, a benzothiophenylene group, a benzosilolylene group, a carbazolylene group, a dibenzofuranylene group, a dibenzothiophenylene group, a dibenzosilolylene group, a benzocarbazolylene group, a benzonaphthofuranylene group, a benzonaphthothiophenylene group, a benzonaphthosilolylene group, a pyridoindolylene group, a benzofuropyridinylene group, a benzothienopyridinylene group, a pyrimidoindolylene group, a benzofuropyrimidinylene group, a benzothionopyrimidinylene group, a phenoxazinylene group, a pyridobenzooxazinylene group, and a pyridobenzothiazinylene group; and
a cyclopentylene group, a cyclohexylene group, a cyclopentenylene group, a cyclohexenylene group, a cycloheptenylene group, a phenylene group, a pentalenylene group, an indenylene group, a naphthylene group, an azulenylene group, a heptalenylene group, an indacenylene group, an acenaphthylene group, a benzoindenylene group, a fluorenylene group, a spiro-bifluorenylene group, a benzofluorenylene group, a dibenzofluorenylene group, a phenalenylene group, a phenanthrenylene group, an anthracenylene group, a fluoranthrenylene group, a triphenylenylene group, a pyrenylene group, a chrysenylene group, a naphthacenylene group, a picenylene group, a perylenylene group, a pentaphenylene group, a hexacenylene group, an imidazolylene group, a pyrazolylene group, a pyridinylene group, a pyrazinylene group, a pyrimidinylene group, a pyridazinylene group, a triazinylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzoxazolylene group, a benzimidazolylene group, a thiazolylene group, an isothiazolylene group, a benzothiazolylene group, an isoxazolylene group, an oxazolylene group, a triazolylene group, a tetrazolylene group, an oxadiazolylene group, an imidazopyrimidinylene group, an imidazopyridinylene group, a pyrrolylene group, a furanylene group, a thiophenylene group, a silolylene group, an isoindolylene group, an indolylene group, a benzofuranylene group, a benzothiophenylene group, a benzosilolylene group, a carbazolylene group, a dibenzofuranylene group, a dibenzothiophenylene group, a dibenzosilolylene group, a benzocarbazolylene group, a benzonaphthofuranylene group, a benzonaphthothiophenylene group, a benzonaphthosilolylene group, a pyridoindolylene group, a benzofuropyridinylene group, a benzothienopyridinylene group, a pyrimidoindolylene group, a benzofuropyrimidinylene group, a benzothienopyrimidinylene group, a phenoxazinylene group, a pyridobenzooxazinylene group, and a pyridobenzothiazinylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a pyridinylphenyl group, a naphthyl group, an anthracenyl group, a pyrenyl group, a phenanthrenyl group, a fluorenyl group, a triphenylenyl group, a pyridinyl group, a phenylpyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, a carbazolyl group, a dibenzofuranyl group, a dibenzothiophenyl group, and —Si(Q₃₃)(Q₃₄)(Q₃₅),
wherein Q₃₃ to Q₃₅ are each independently selected from a C₁-C₂₀ alkyl group, a C₁-C₂₀ alkoxy group, a phenyl group, a biphenyl group, a terphenyl group, a pyridinylphenyl group, a naphthyl group, a pyridinyl group, and a phenylpyridinyl group.

14. The organic light-emitting device of claim 1, wherein
L₅₁ to L₅₃, L₆₁, L₆₂, and to L₉₅ in Formulae 51, 61, 61B, and 91 are each independently selected from a single bond and groups represented by Formulae 4-1 to 4-36: 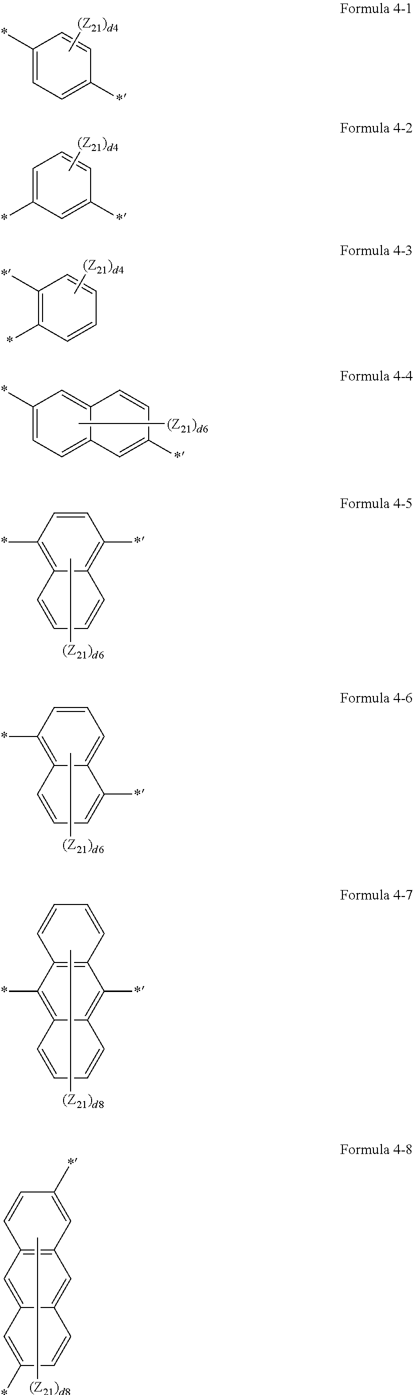 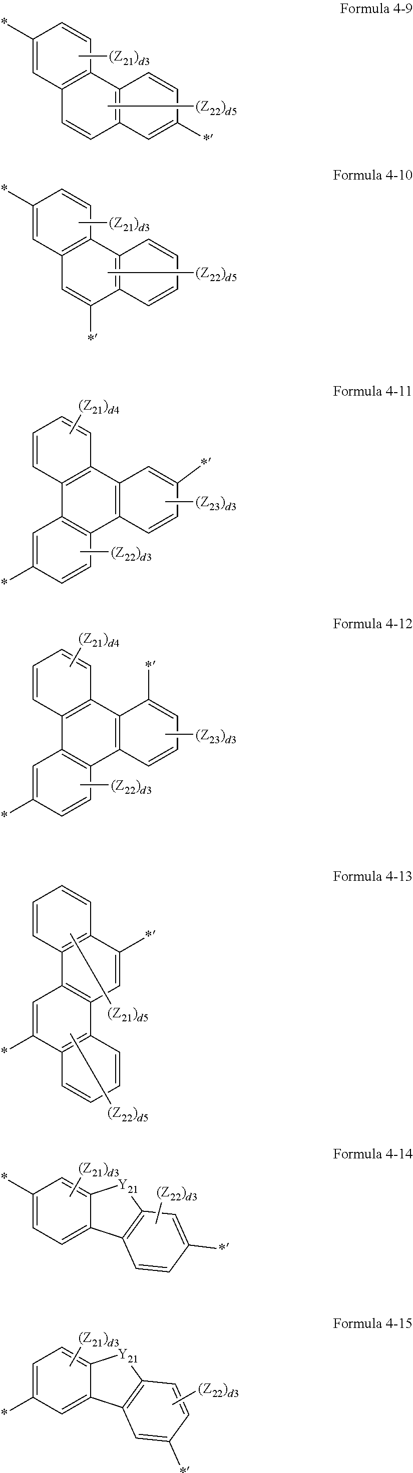 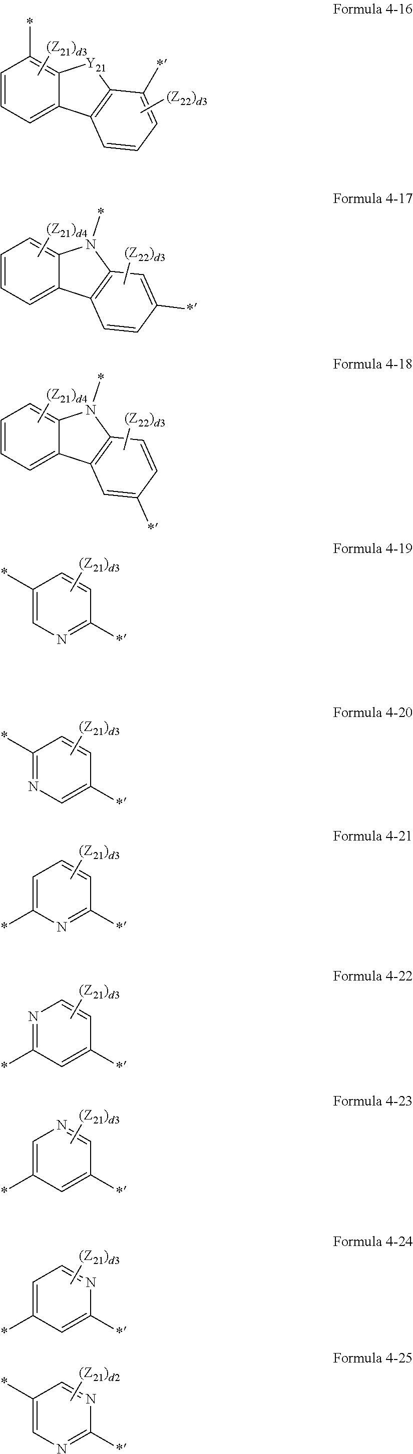 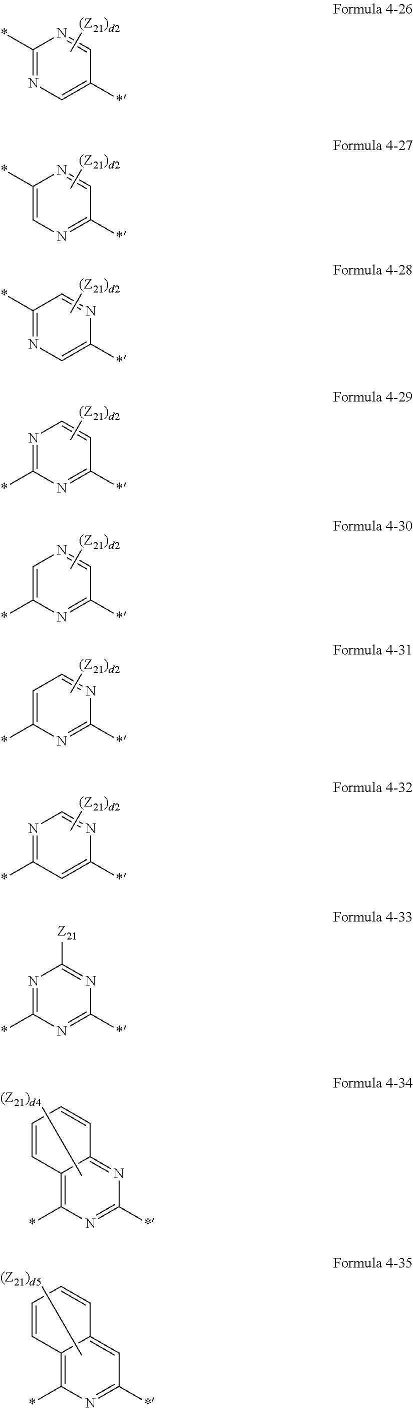 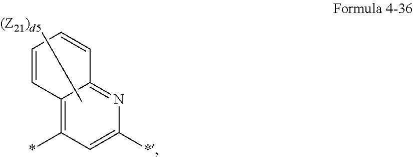
wherein, in Formulae 4-1 to 4-36,
Y₂₁ is O, S, N(Z₂₃), C(Z₂₃)(Z₂₄), or Si(Z₂₃)(Z₂₄),
Z₂₁ to Z₂₄ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a pyridinylphenyl group, a naphthyl group, an anthracenyl group, a pyrenyl group, a phenanthrenyl group, a fluorenyl group, a triphenylenyl group, a pyridinyl group, a phenylpyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, a carbazolyl group, a dibenzofuranyl group, a dibenzothiophenyl group, and —Si(Q₃₃)(Q₃₄)(Q₃₅),
wherein Q₃₃ to Q₃s are each independently selected from a C₁-C₂₀ alkyl group, a C₁-C₂₀ alkoxy group, a phenyl group, a biphenyl group, a terphenyl group, a pyridinylphenyl group, a naphthyl group, a pyridinyl group, and a phenylpyridinyl group,
d2 is an integer selected from 0 to 2,
d3 is an integer selected from 0 to 3,
d4 is an integer selected from 0 to 4,
d5 is an integer selected from 0 to 5,
d6 is an integer selected from 0 to 6,
d8 is an integer selected from 0 to 8, and
* and *′ each indicate a binding site to a neighboring atom.

15. The organic light-emitting device of claim 1, wherein
R₅₁ to R₅₃, R₆₁ to R₆₄, R₇₁ to R₇₉, and R₉₁ to R₉₅ in Formulae 51, 61, 61A, 61B and 91 are each independently selected from:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;
a C₁-C₂₀ alkyl group and a C₁-C₂₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;
a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a benzoindenyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, an imidazolyl group, a pyrazolyl group, a pyridinyl group, a pyrazinyl group, a pyrimidinyl group, a pyridazinyl group, a triazinyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a thiazolyl group, an isothiazolyl group, a benzothiazolyl group, an isoxazolyl group, an oxazolyl group, a triazolyl group, a tetrazolyl group, an oxadiazolyl group, an imidazopyrimidinyl group, an imidazopyridinyl group, a pyrrolyl group, a furanyl group, a thiophenyl group, a silolyl group, an isoindolyl group, an indolyl group, a benzofuranyl group, a benzothiophenyl group, a benzosilolyl group, a carbazolyl group, a dibenzofuranyl group, a dibenzothiophenyl group, a dibenzosilolyl group, a benzocarbazolyl group, a benzonaphthofuranyl group, a benzonaphthothiophenyl group, a benzonaphthosilolyl group, a pyridoindolyl group, a benzofuropyridinyl group, a benzothienopyridinyl group, a pyrimidoindolyl group, a benzofuropyrimidinyl group, a benzothienopyrimidinyl group, a phenoxazinyl group, a pyridobenzooxazinyl group, and a pyridobenzothiazinyl group;
a cyclopentyl group, a cyclohexyl group, a cyclopentenyl group, a cyclohexenyl group, a cycloheptenyl group, a phenyl group, a pentalenyl group, an indenyl group, a naphthyl group, an azulenyl group, a heptalenyl group, an indacenyl group, an acenaphthyl group, a benzoindenyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, an imidazolyl group, a pyrazolyl group, a pyridinyl group, a pyrazinyl group, a pyrimidinyl group, a pyridazinyl group, a triazinyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzimidazolyl group, a thiazolyl group, an isothiazolyl group, a benzothiazolyl group, an isoxazolyl group, an oxazolyl group, a triazolyl group, a tetrazolyl group, an oxadiazolyl group, an imidazopyrimidinyl group, an imidazopyridinyl group, a pyrrolyl group, a furanyl group, a thiophenyl group, a silolyl group, an isoindolyl group, an indolyl group, a benzofuranyl group, a benzothiophenyl group, a benzosilolyl group, a carbazolyl group, a dibenzofuranyl group, a dibenzothiophenyl group, a dibenzosilolyl group, a benzocarbazolyl group, a benzonaphthofuranyl group, a benzonaphthothiophenyl group, a benzonaphthosilolyl group, a pyridoindolyl group, a benzofuropyridinyl group, a benzothienopyridinyl group, a pyrimidoindolyl group, a benzofuropyrimidinyl group, a benzothienopyrimidinyl group, a phenoxazinyl group, a pyridobenzooxazinyl group, and a pyrimidobenzothiazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a pyridinylphenyl group, a naphthyl group, an anthracenyl group, a pyrenyl group, a phenanthrenyl group, a fluorenyl group, a triphenylenyl group, a pyridinyl group, a phenylpyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, a carbazolyl group, a dibenzofuranyl group, a dibenzothiophenyl group, and —Si(Q₃₃)(Q₃₄)(Q₃₅); and
—Si(Q₁₃)(Q₁₄)(Q₁₅),
wherein Q₁₃ to Q₁₅ and Q₃₃ to Q₃₅ are each independently selected from a C₁-C₂₀ alkyl group, a C₁-C₂₀ alkoxy group, a phenyl group, a biphenyl group, a terphenyl group, a pyridinylphenyl group, a naphthyl group, and a pyridinyl group, and a phenylpyridinyl group.

16. The organic light-emitting device of claim 1, wherein
R₅₁ to R₅₃, R₆₁ to R₆₄, R₇₁ to R₇₉, and R₉₁ to R₉₅ in Formulae 51, 61, 61A, 61B, and 91 are each independently selected from groups represented by Formulae 5-1 to 5-15, 6-1 to 6-6, and 7-1 to 7-50: 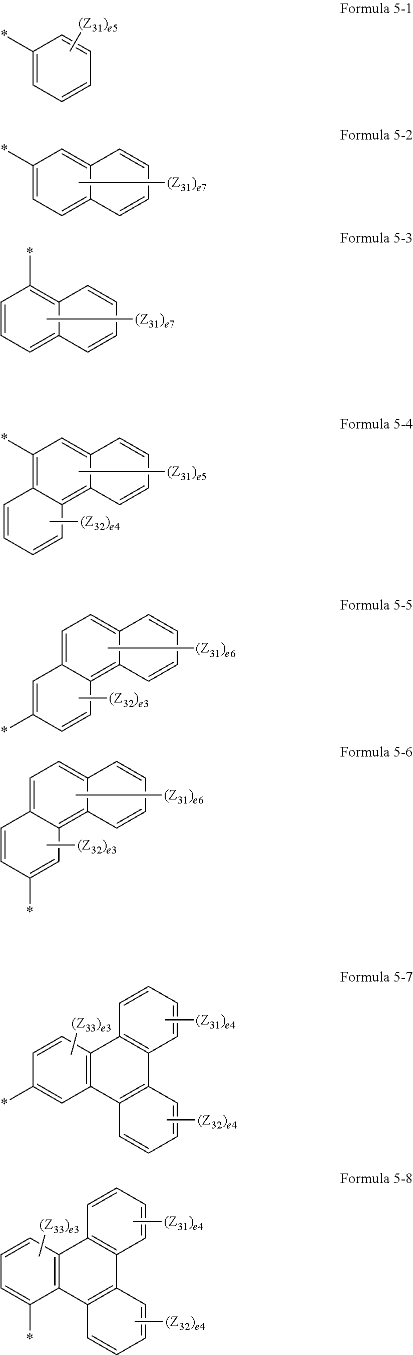 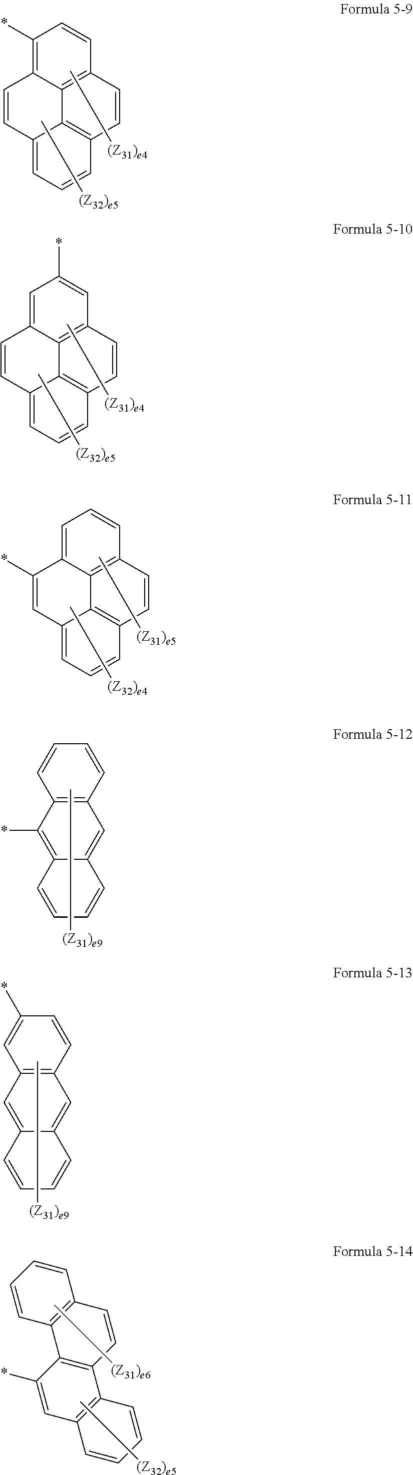 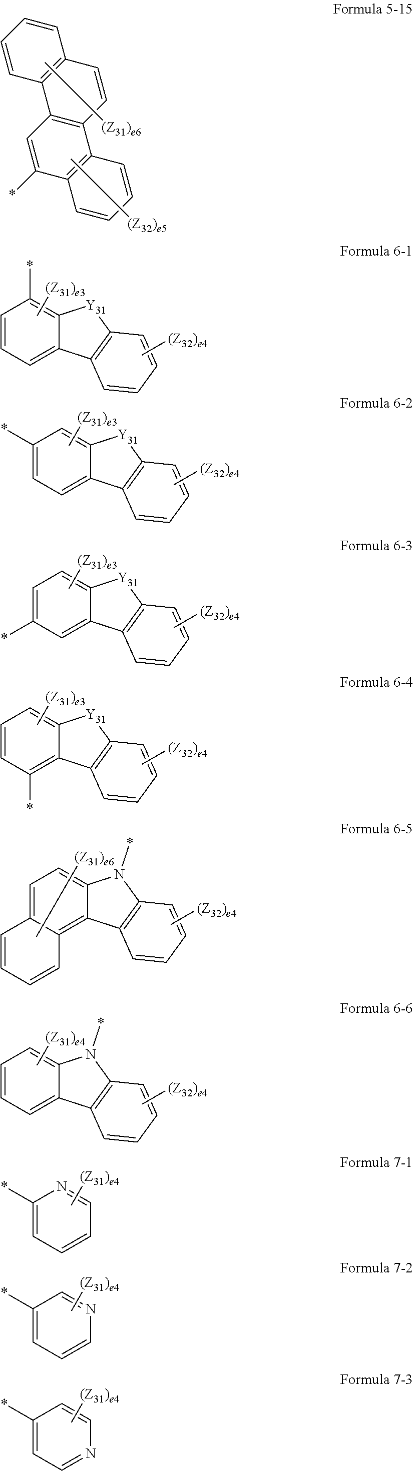 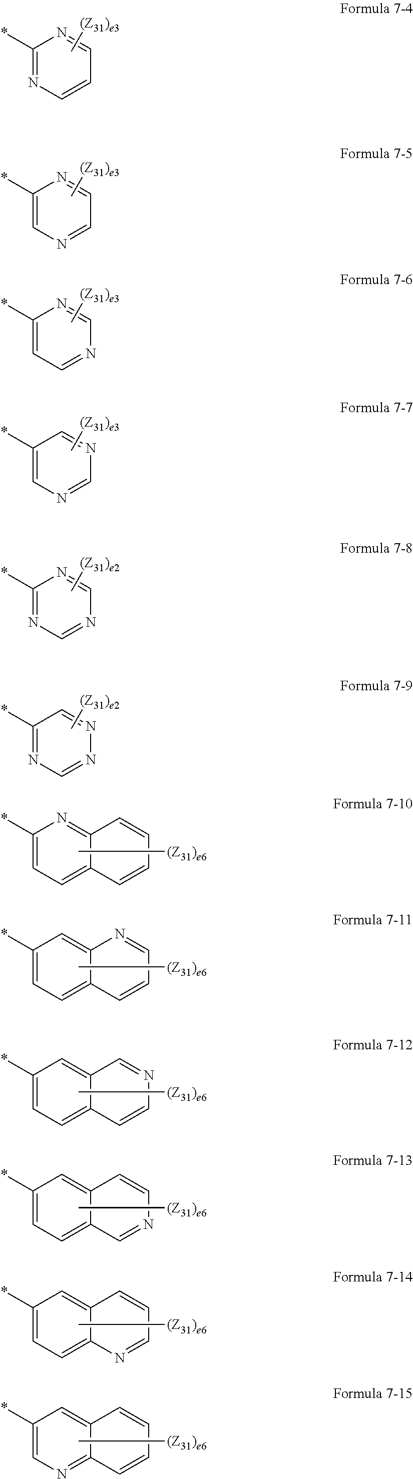 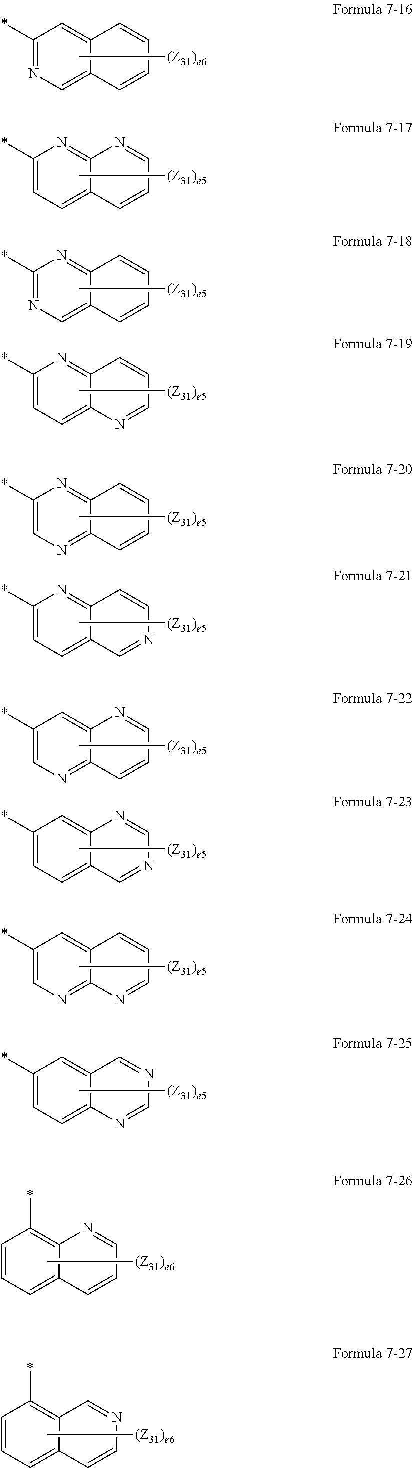 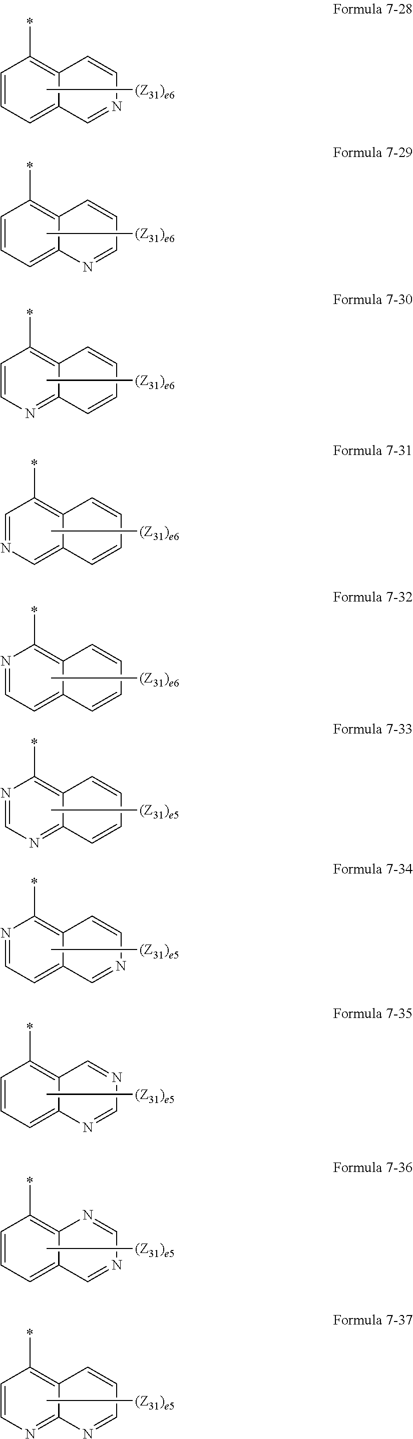 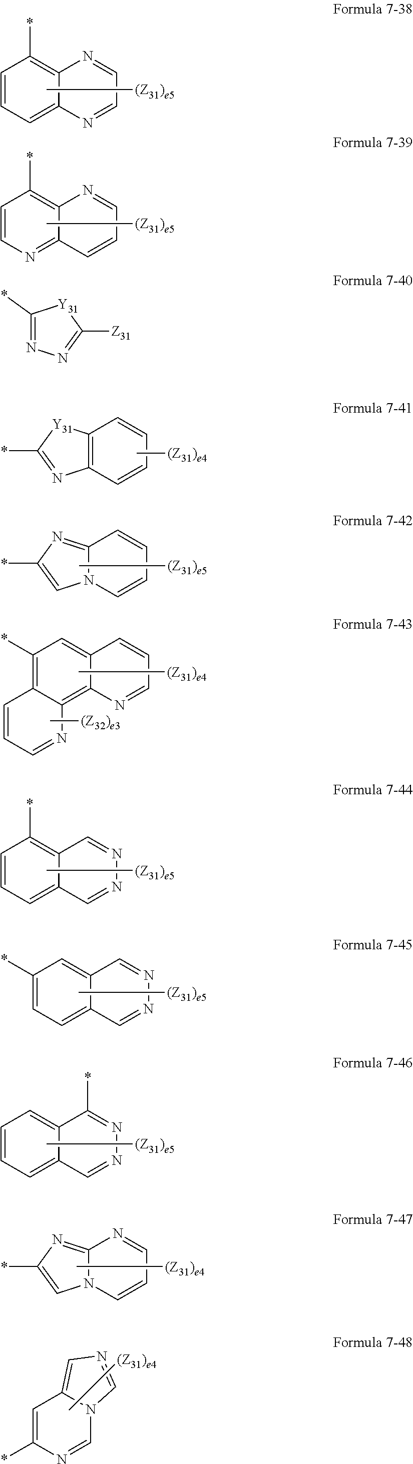 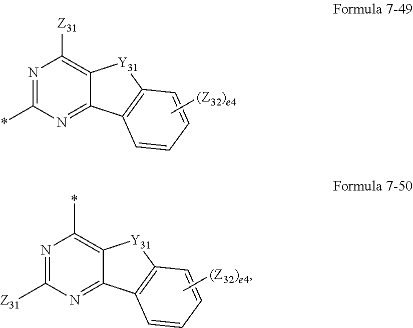
wherein, in Formulae 5-1 to 5-15, 6-1 to 6-6, and 7-1 to 7-50,
Y₃₁ is O, S, N(Z₃₃), C(Z₃₃)(Z₃₄), or Si(Z₃₃)(Z₃₄),
Z₃₁ to Z₃₄ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a pyridinylphenyl group, a naphthyl group, an anthracenyl group, a pyrenyl group, a phenanthrenyl group, a fluorenyl group, a triphenylenyl group, a pyridinyl group, a phenylpyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, a carbazolyl group, a dibenzofuranyl group, a dibenzothiophenyl group, and —Si(Q₃₃)(Q₃₄)(Q₃₅),
wherein Q₃₃ to Q₃₅ are each independently selected from a C₁-C₂₀ alkyl group, a C₁-C₂₀ alkoxy group, a phenyl group, a biphenyl group, a terphenyl group, a pyridinylphenyl group, a naphthyl group, and a pyridinyl group, and a phenylpyridinyl group,
e2 is an integer selected from 0 to 2,
e3 is an integer selected from 0 to 3,
e4 is an integer selected from 0 to 4,
e5 is an integer selected from 0 to 5,
e6 is an integer selected from 0 to 6,
e1 is an integer selected from 0 to 7,
e9 is an integer selected from 0 to 9, and
* indicates a binding site to a neighboring atom.

17. The organic light-emitting device of claim 1, wherein
the first compound is represented by one selected from Formulae 51-1 to 51-3,
the second compound is represented by one selected from Formulae 61-1 to 61-6,
the fourth compound is represented by one selected from Formulae 91-1 to 91-14: 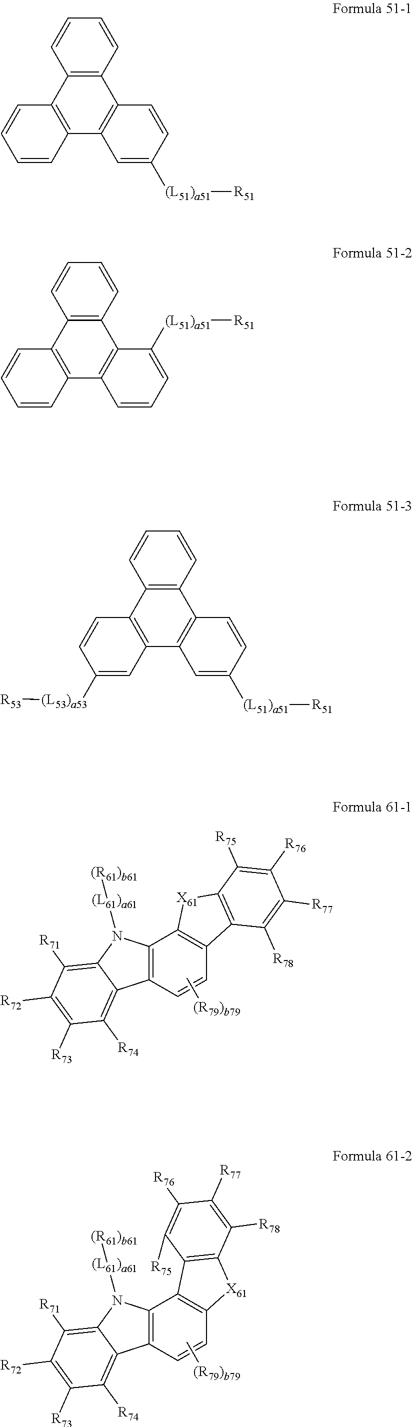 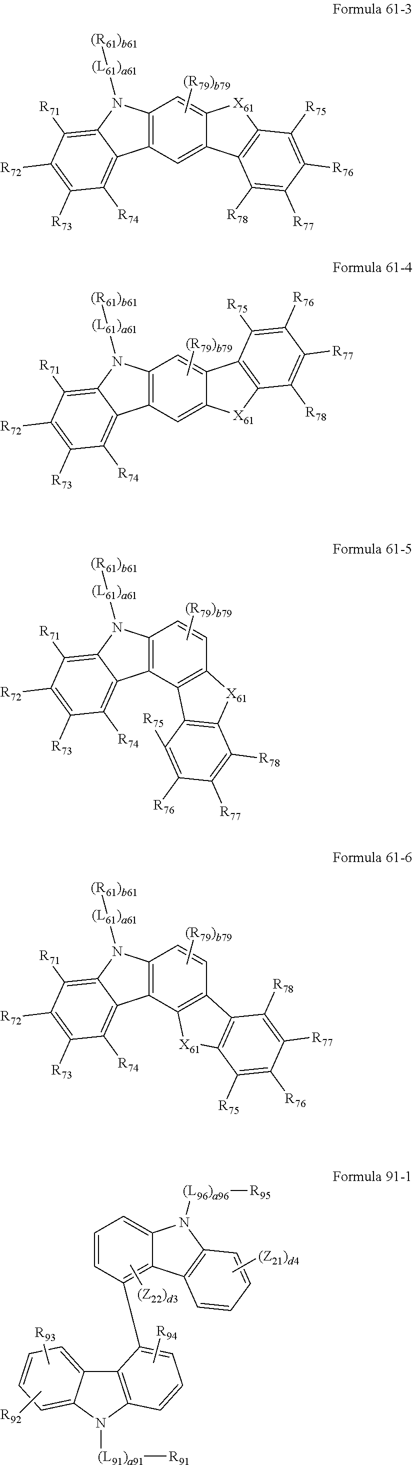 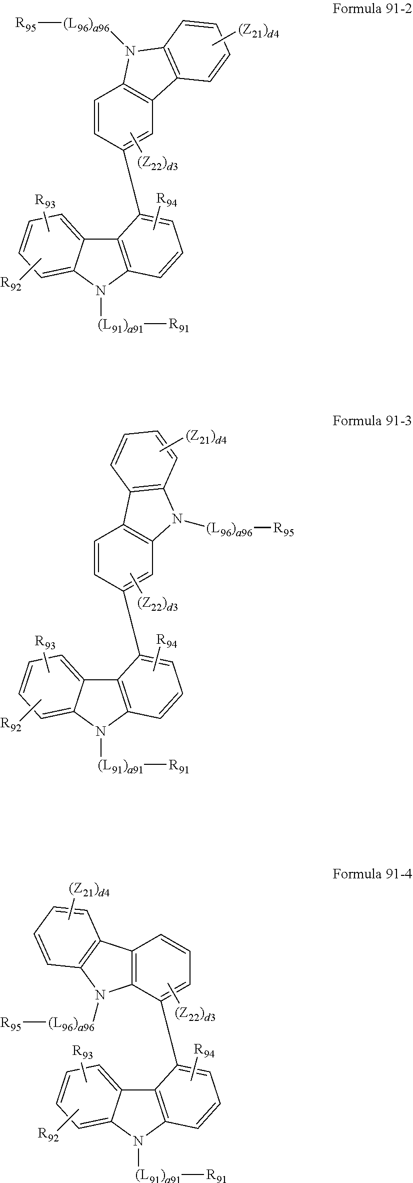 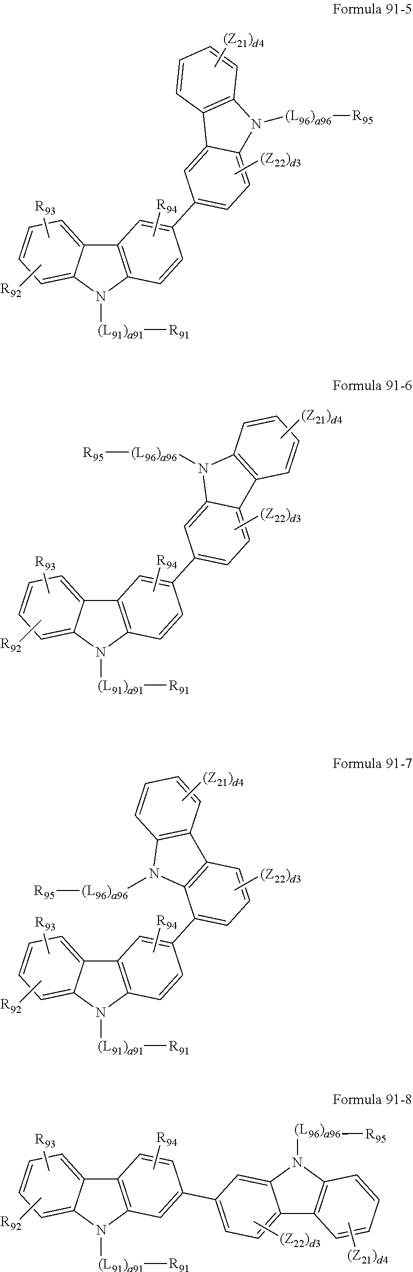 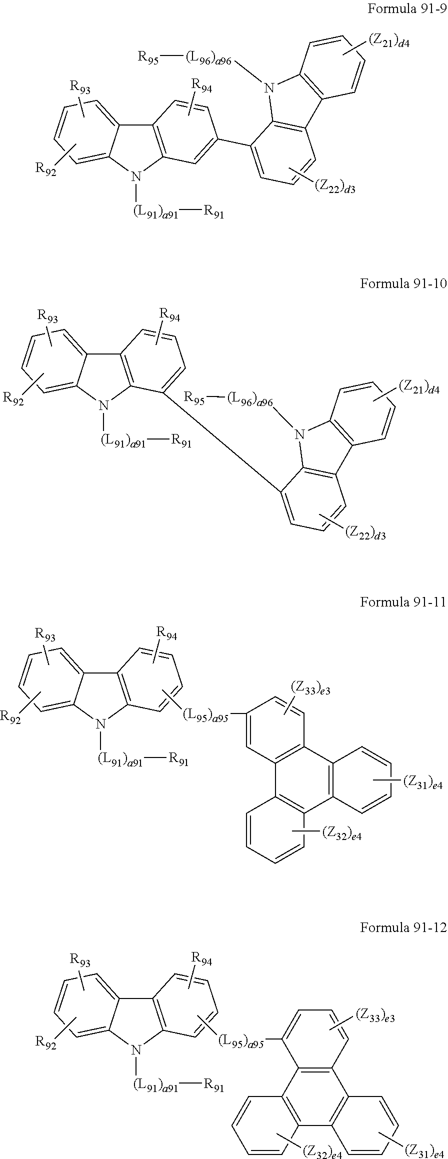 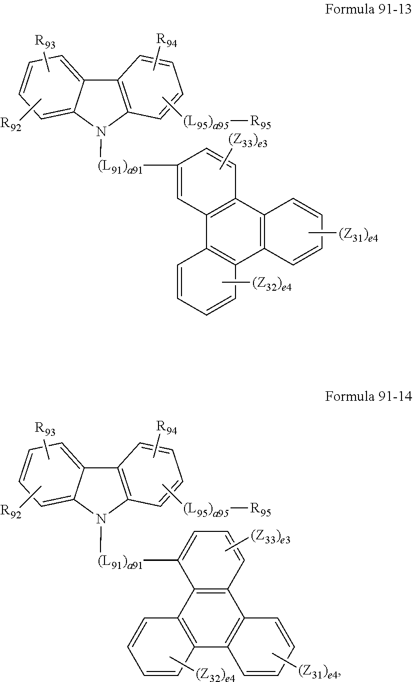
wherein L₅₁, L₅₃, a51, a53, R₅₁, and R₅₃ in Formulae 51-1 to 51-3 are the same as in claim 1,
X₆₁, L₆₁, a61, R₆₁, b61, R₇₁ to R₇₉, and b79 in Formulae 61-1 to 61-6 are the same as in claim 1,
in Formulae 91-1 to 91-14,
L₉₁, a91, and R₉₁ to R₉₅ are the same as in claim 1,
L₉₆ is the same as in connection with L₉₁,
a96 is 1 or 2,
Z₃₁ to Z₃₃ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a pyridinylphenyl group, a naphthyl group, an anthracenyl group, a pyrenyl group, a phenanthrenyl group, a fluorenyl group, a triphenylenyl group, a pyridinyl group, a phenylpyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, a quinazolinyl group, a carbazolyl group, a dibenzofuranyl group, a dibenzothiophenyl group, and —Si(Q₃₃)(Q₃₄)(Q₃₅),
wherein Q₃₃ to Q₃₅ are each independently selected from a C₁-C₂₀ alkyl group, a C₁-C₂₀ alkoxy group, a phenyl group, a biphenyl group, a terphenyl group, a pyridinylphenyl group, a naphthyl group, a pyridinyl group, and a phenylpyridinyl group,
e3 is an integer selected from 0 to 3, and
e4 is an integer selected from 0 to 4.

18. The organic light-emitting device of claim 1, wherein
a82 and a83 in Formula 81 are each independently 0 or 1, wherein at least one selected from a82 and a83 is 1,
L₈₁ in Formula 81 is one selected from groups represented by Formulae 8-1 to 8-36: 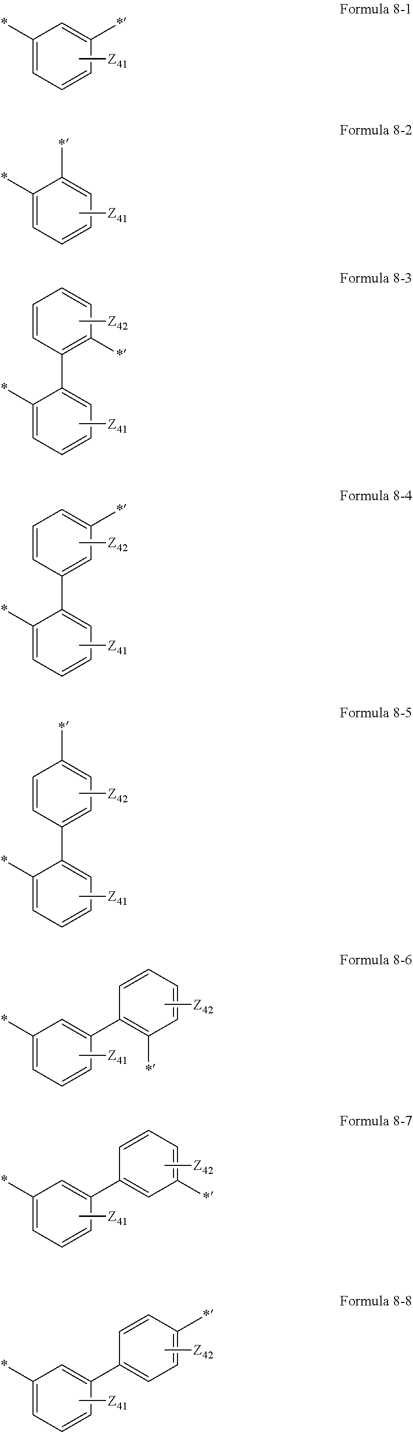 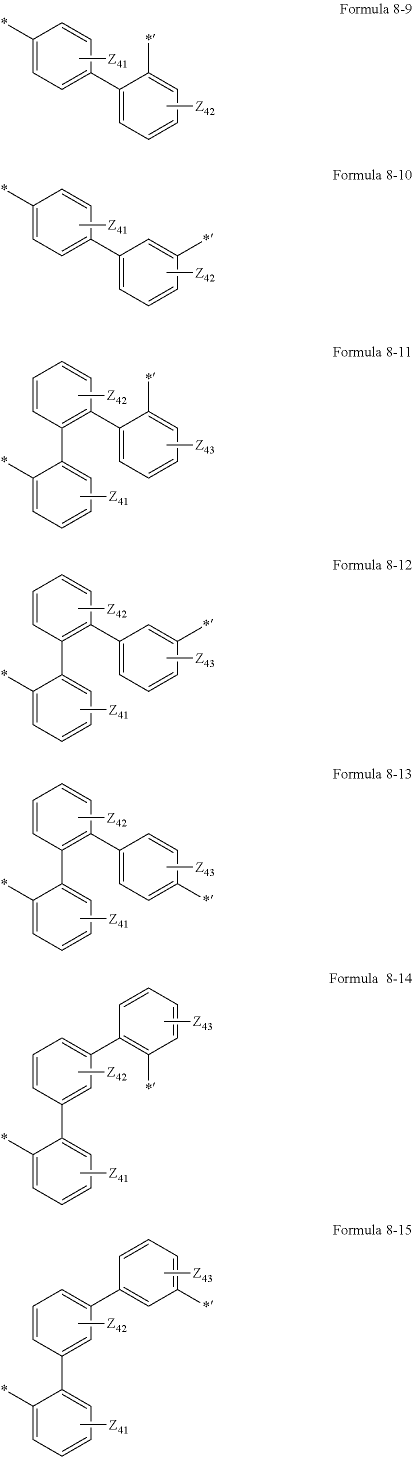 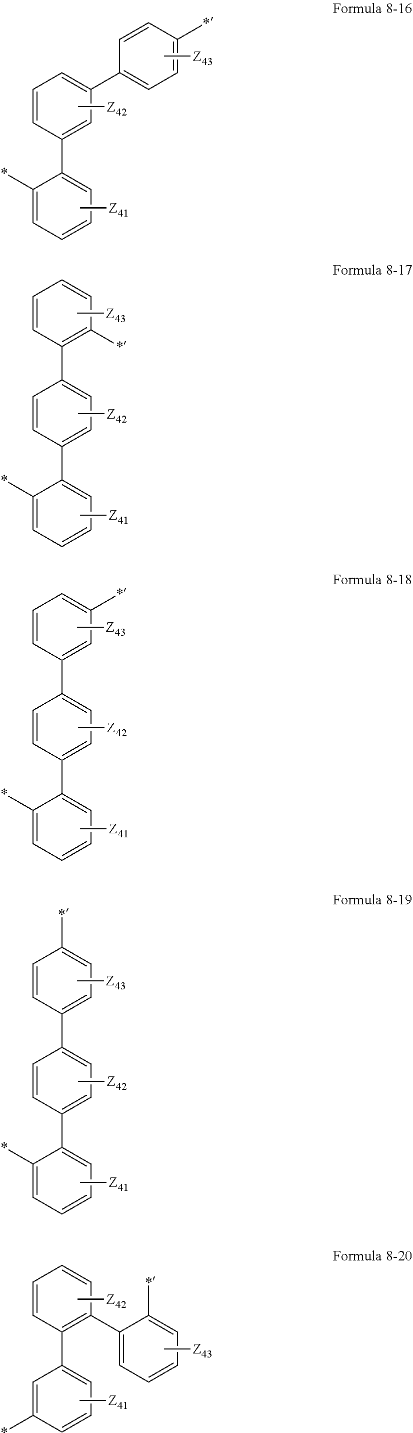 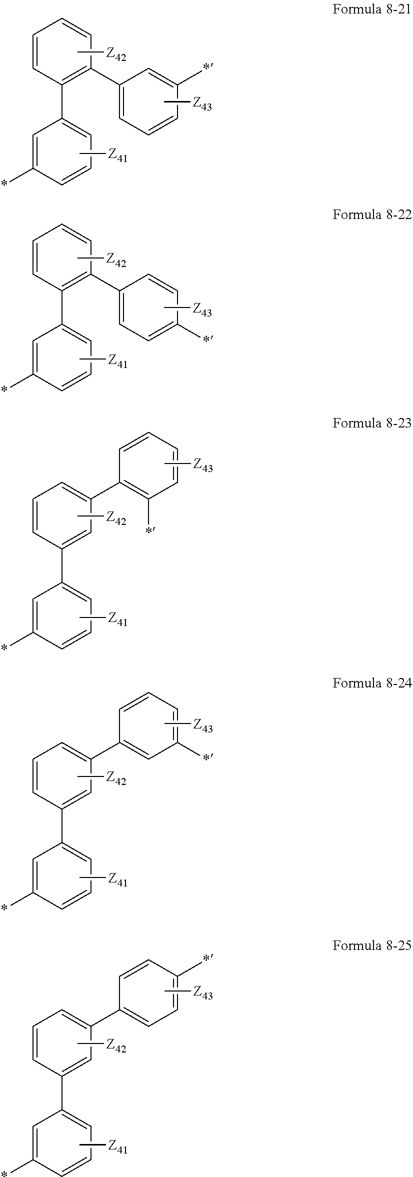 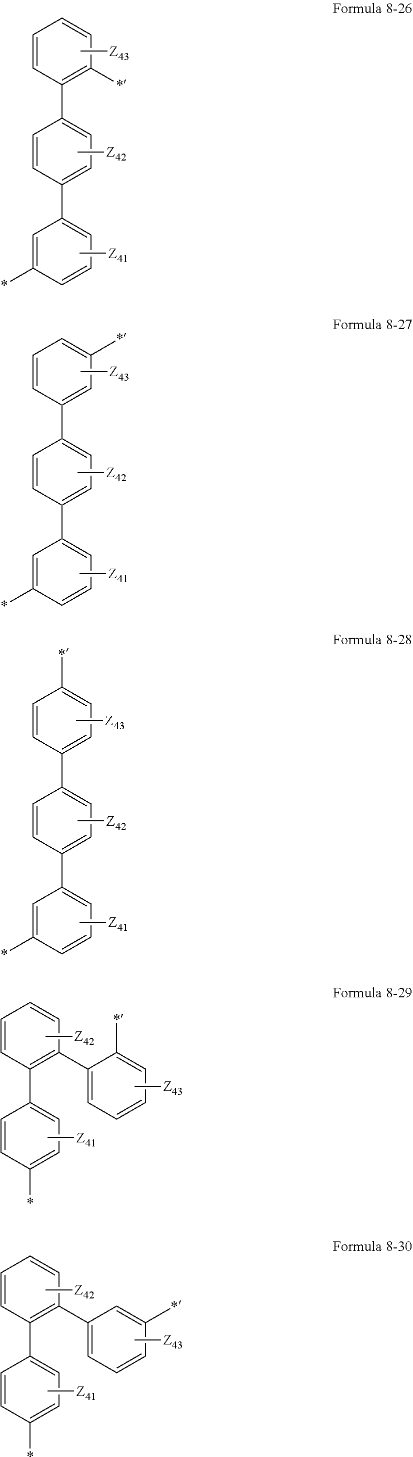 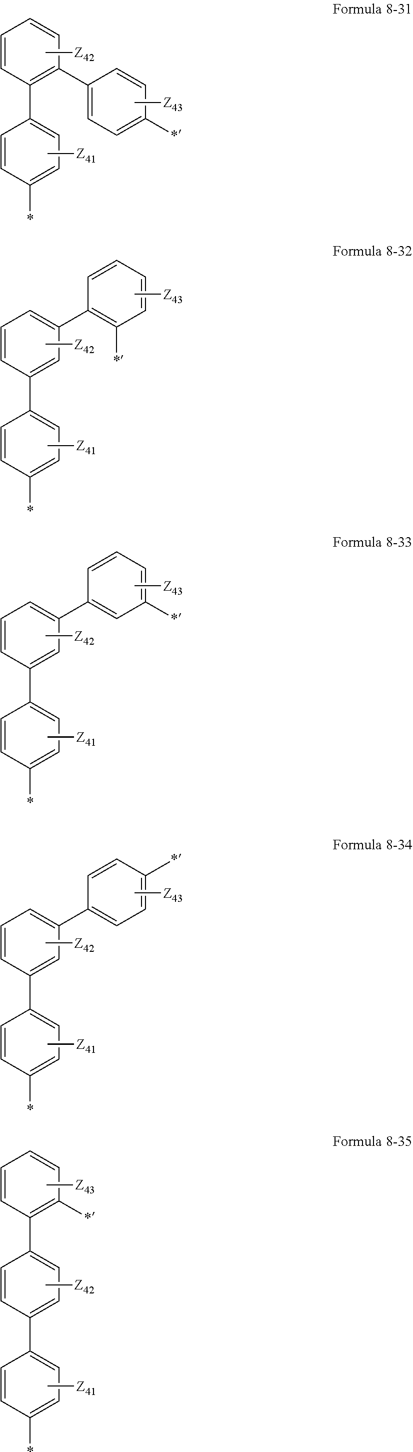 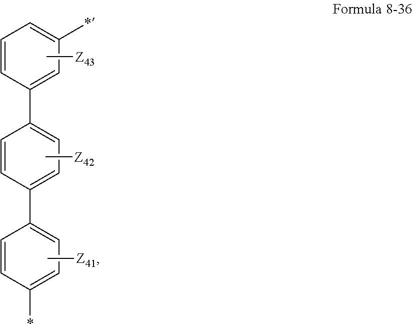
wherein Z₄₁ to Z₄₃ in Formulae 8-1 to 8-36 are the same as in connection with R₈₁ in Formula 81, and * and *′ each indicate a binding site to a neighboring atom.

19. The organic light-emitting device of claim 1, wherein
the first compound is selected from Compounds A1 to A18,
the second compound is selected from Compounds B1 to B30,
the third compound is selected from Compounds A-1 to A-125,
the fourth compound is selected from Compounds B-10 to B-161, C-10 to C-33, and D-10 to D-29:

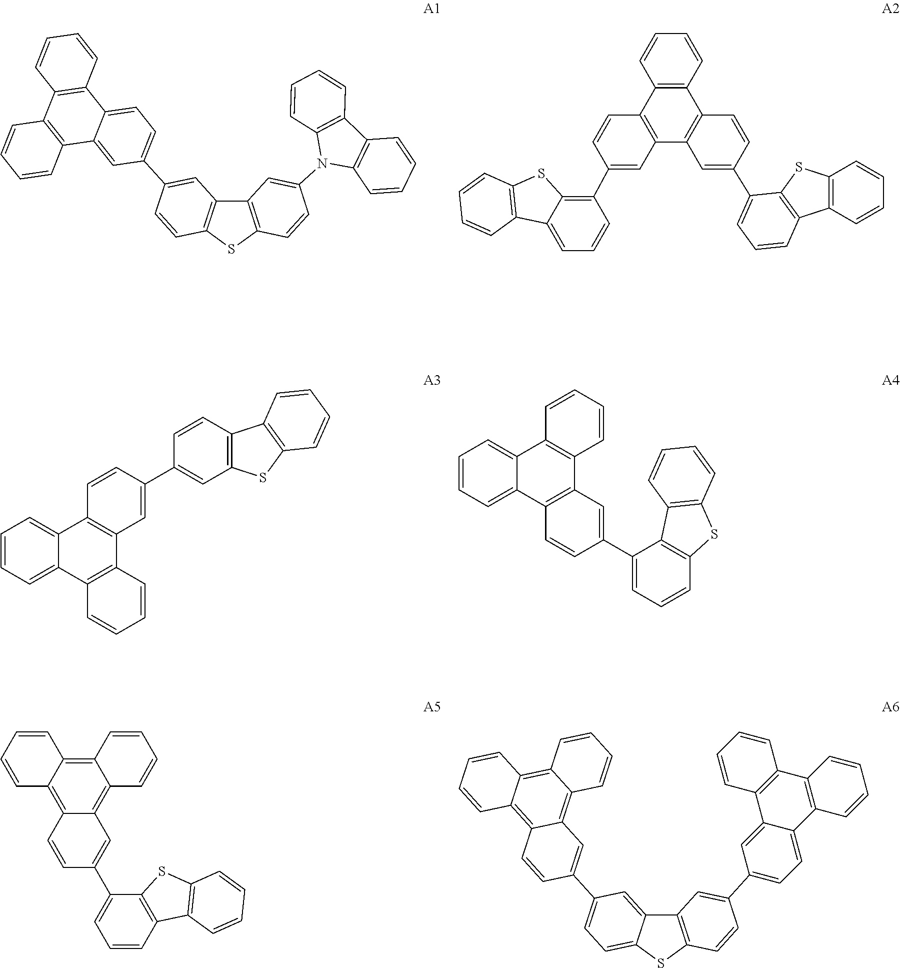 [SMILES: c1ccc2c(c1)c1ccccc1c1cc(-c3ccc4sc5ccc(-n6c7ccccc7c7ccccc76)cc5c4c3)ccc21 ; c1ccc2c(c1)sc1c(-c3ccc4c5ccccc5c5ccc(-c6cccc7c6sc6ccccc67)cc5c4c3)cccc12 ; c1ccc2c(c1)sc1cc(-c3ccc4c5ccccc5c5ccccc5c4c3)ccc12 ; c1ccc2c(c1)sc1cccc(-c3ccc4c5ccccc5c5ccccc5c4c3)c12 ; c1ccc2c(c1)sc1c(-c3ccc4c5ccccc5c5ccccc5c4c3)cccc12 ; c1ccc2c(c1)c1ccccc1c1cc(-c3ccc4sc5ccc(-c6ccc7c8ccccc8c8ccccc8c7c6)cc5c4c3)ccc21]

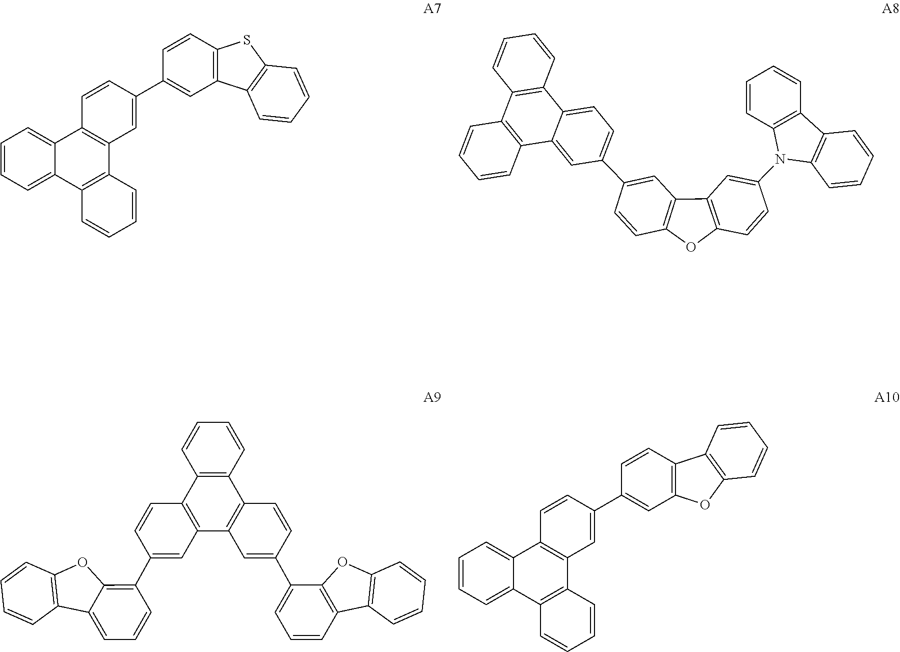

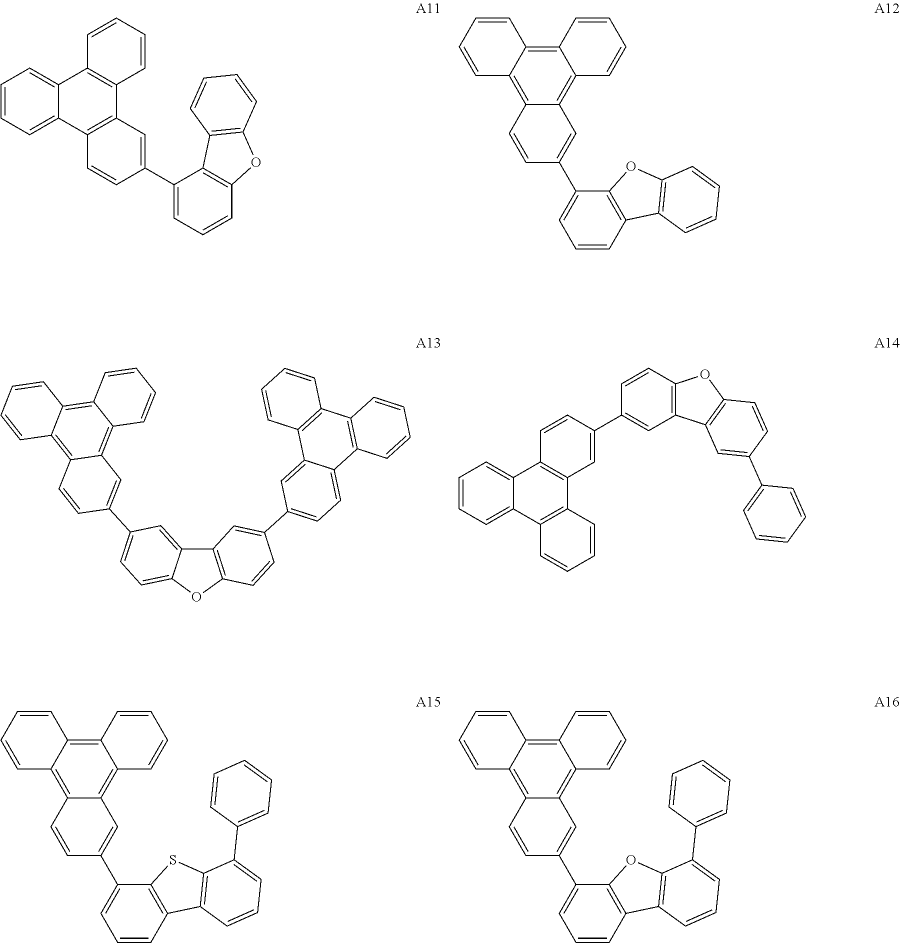 [SMILES: c1ccc2c(c1)oc1cccc(-c3ccc4c5ccccc5c5ccccc5c4c3)c12 ; c1ccc2c(c1)oc1c(-c3ccc4c5ccccc5c5ccccc5c4c3)cccc12 ; c1ccc2c(c1)c1ccccc1c1cc(-c3ccc4oc5ccc(-c6ccc7c8ccccc8c8ccccc8c7c6)cc5c4c3)ccc21 ; c1ccc(-c2ccc3oc4ccc(-c5ccc6c7ccccc7c7ccccc7c6c5)cc4c3c2)cc1 ; c1ccc(-c2cccc3c2sc2c(-c4ccc5c6ccccc6c6ccccc6c5c4)cccc23)cc1 ; c1ccc(-c2cccc3c2oc2c(-c4ccc5c6ccccc6c6ccccc6c5c4)cccc23)cc1]

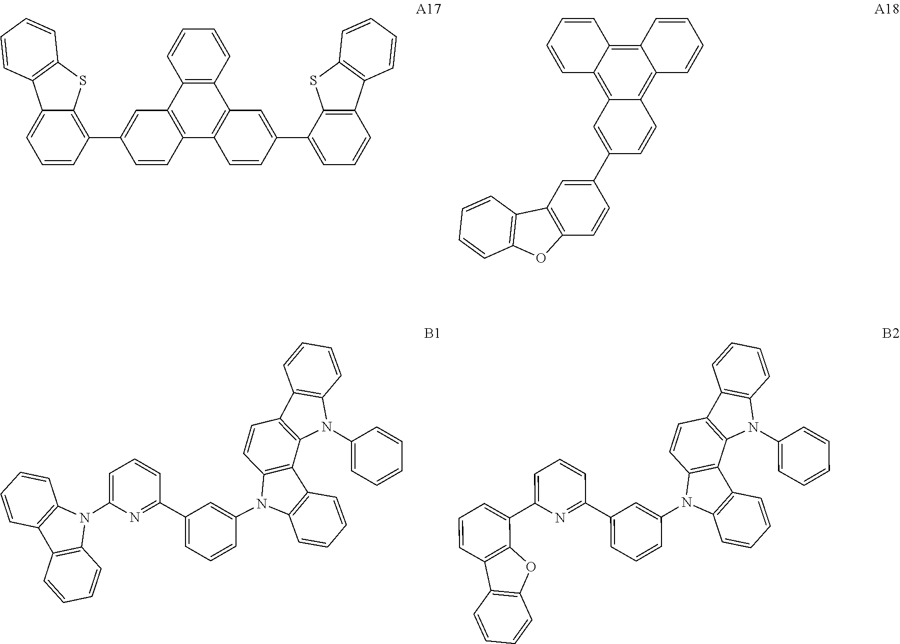 [SMILES: c1ccc2c(c1)sc1c(-c3ccc4c5ccc(-c6cccc7c6sc6ccccc67)cc5c5ccccc5c4c3)cccc12 ; c1ccc2c(c1)oc1ccc(-c3ccc4c5ccccc5c5ccccc5c4c3)cc12 ; c1ccc(-n2c3ccccc3c3ccc4c(c5ccccc5n4-c4cccc(-c5cccc(-n6c7ccccc7c7ccccc76)n5)c4)c32)cc1 ; c1ccc(-n2c3ccccc3c3ccc4c(c5ccccc5n4-c4cccc(-c5cccc(-c6cccc7c6oc6ccccc67)n5)c4)c32)cc1]

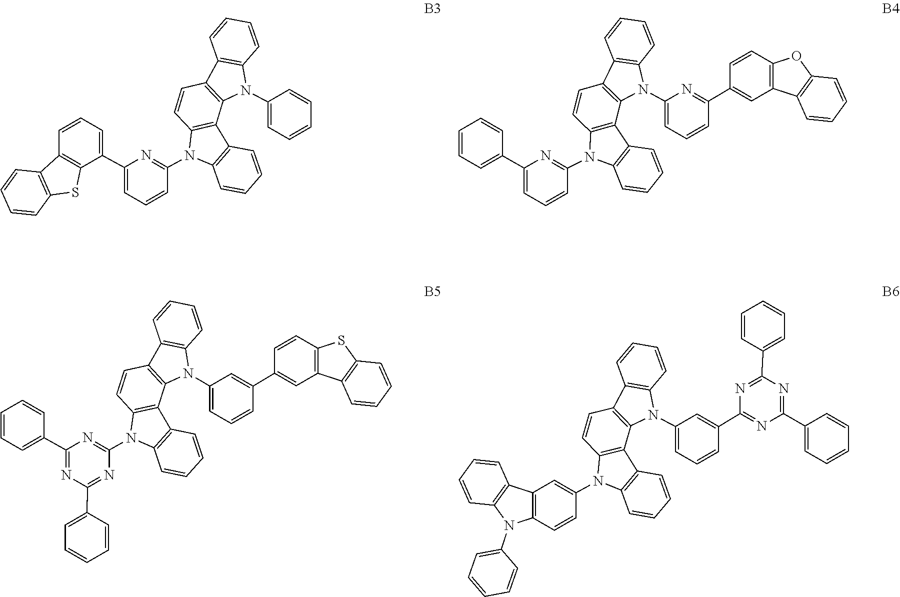 [SMILES: c1ccc(-n2c3ccccc3c3ccc4c(c5ccccc5n4-c4cccc(-c5cccc6c5sc5ccccc56)n4)c32)cc1 ; c1ccc(-c2cccc(-n3c4ccccc4c4c3ccc3c5ccccc5n(-c5cccc(-c6ccc7oc8ccccc8c7c6)n5)c34)n2)cc1 ; c1ccc(-c2nc(-c3ccccc3)nc(-n3c4ccccc4c4c3ccc3c5ccccc5n(-c5cccc(-c6ccc7sc8ccccc8c7c6)c5)c34)n2)cc1 ; c1ccc(-c2nc(-c3ccccc3)nc(-c3cccc(-n4c5ccccc5c5ccc6c(c7ccccc7n6-c6ccc7c(c6)c6ccccc6n7-c6ccccc6)c54)c3)n2)cc1]

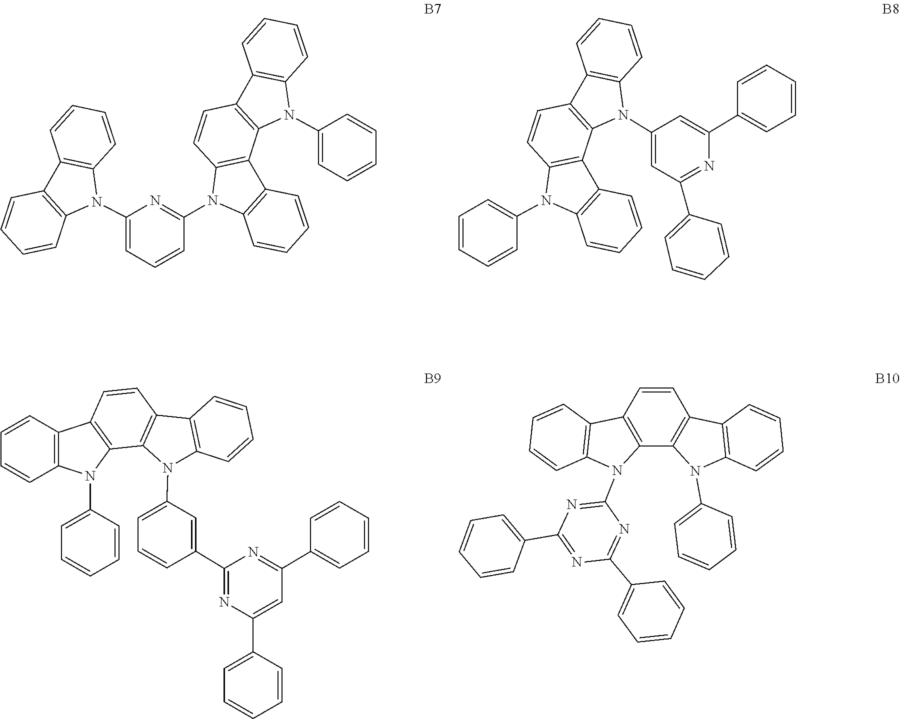 [SMILES: c1ccc(-n2c3ccccc3c3ccc4c(c5ccccc5n4-c4cccc(-n5c6ccccc6c6ccccc65)n4)c32)cc1 ; c1ccc(-c2cc(-n3c4ccccc4c4ccc5c(c6ccccc6n5-c5ccccc5)c43)cc(-c3ccccc3)n2)cc1 ; c1ccc(-c2cc(-c3ccccc3)nc(-c3cccc(-n4c5ccccc5c5ccc6c7ccccc7n(-c7ccccc7)c6c54)c3)n2)cc1 ; c1ccc(-c2nc(-c3ccccc3)nc(-n3c4ccccc4c4ccc5c6ccccc6n(-c6ccccc6)c5c43)n2)cc1]

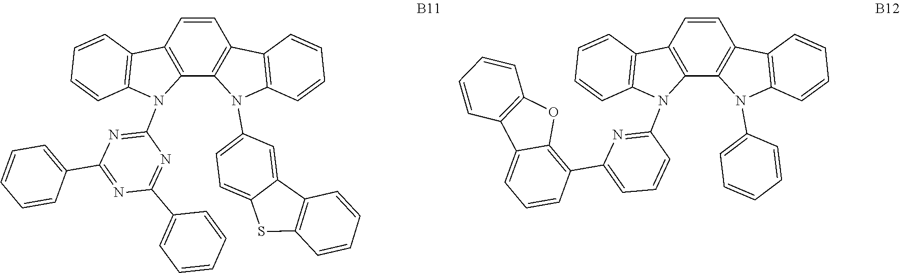 [SMILES: c1ccc(-c2nc(-c3ccccc3)nc(-n3c4ccccc4c4ccc5c6ccccc6n(-c6ccc7sc8ccccc8c7c6)c5c43)n2)cc1 ; c1ccc(-n2c3ccccc3c3ccc4c5ccccc5n(-c5cccc(-c6cccc7c6oc6ccccc67)n5)c4c32)cc1]

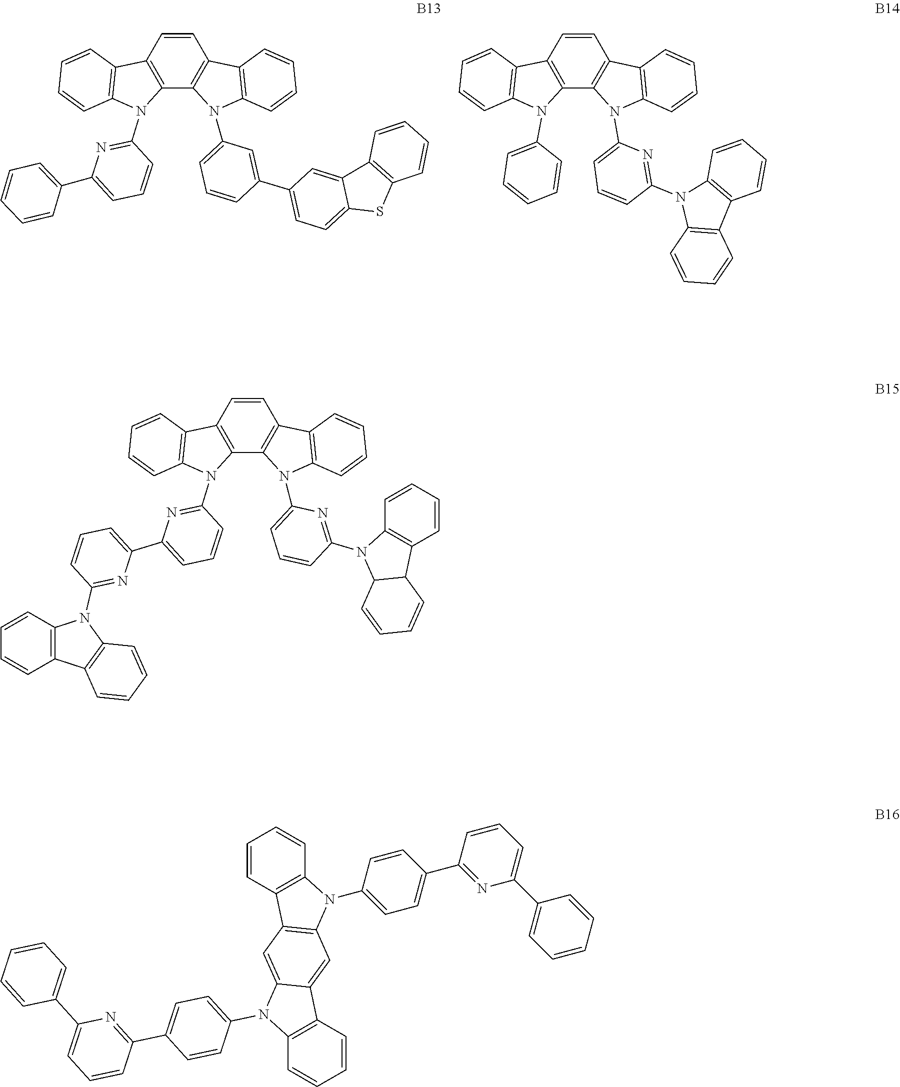 [SMILES: c1ccc(-c2cccc(-n3c4ccccc4c4ccc5c6ccccc6n(-c6cccc(-c7ccc8sc9ccccc9c8c7)c6)c5c43)n2)cc1 ; c1ccc(-n2c3ccccc3c3ccc4c5ccccc5n(-c5cccc(-n6c7ccccc7c7ccccc76)n5)c4c32)cc1 ; C1=CC2c3ccccc3N(c3cccc(-n4c5ccccc5c5ccc6c7ccccc7n(-c7cccc(-c8cccc(-n9c%10ccccc%10c%10ccccc%109)n8)n7)c6c54)n3)C2C=C1 ; c1ccc(-c2cccc(-c3ccc(-n4c5ccccc5c5cc6c(cc54)c4ccccc4n6-c4ccc(-c5cccc(-c6ccccc6)n5)cc4)cc3)n2)cc1]

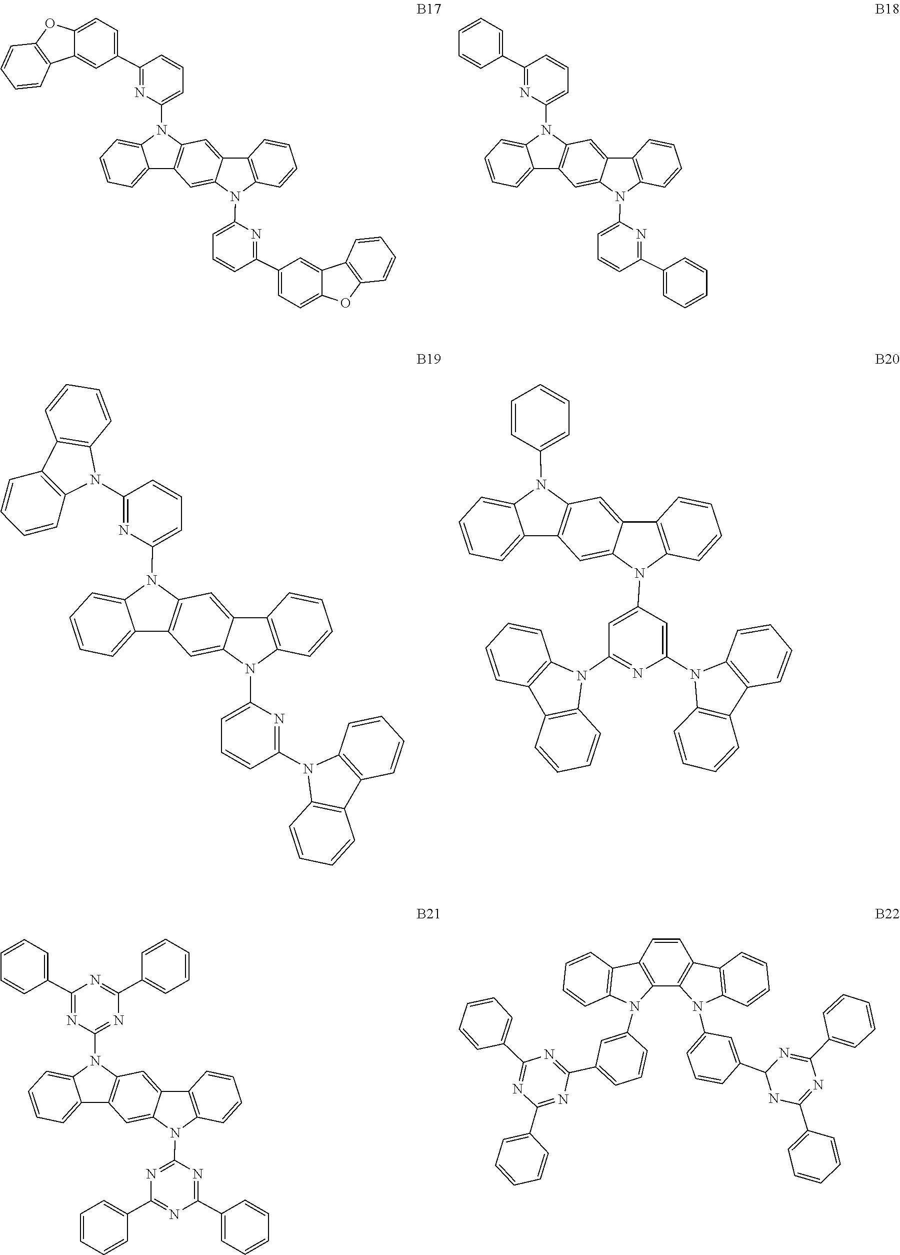

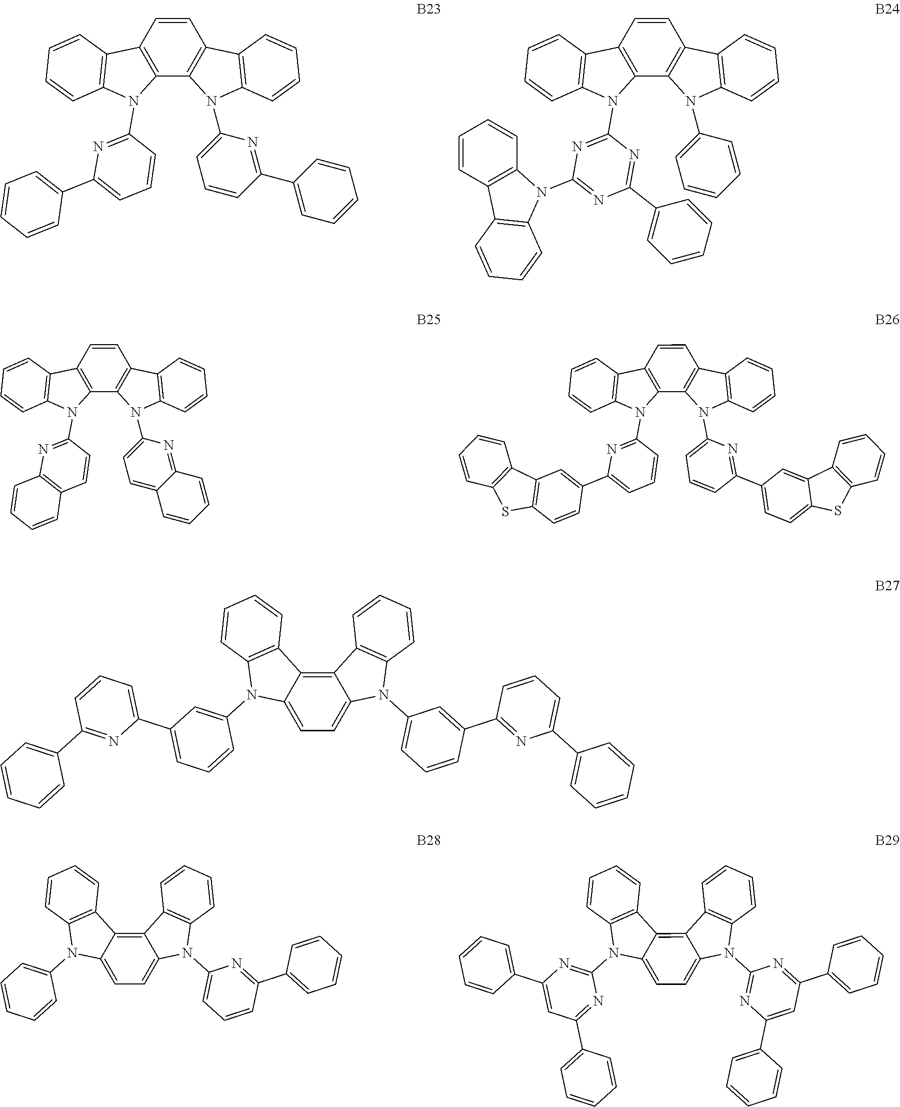

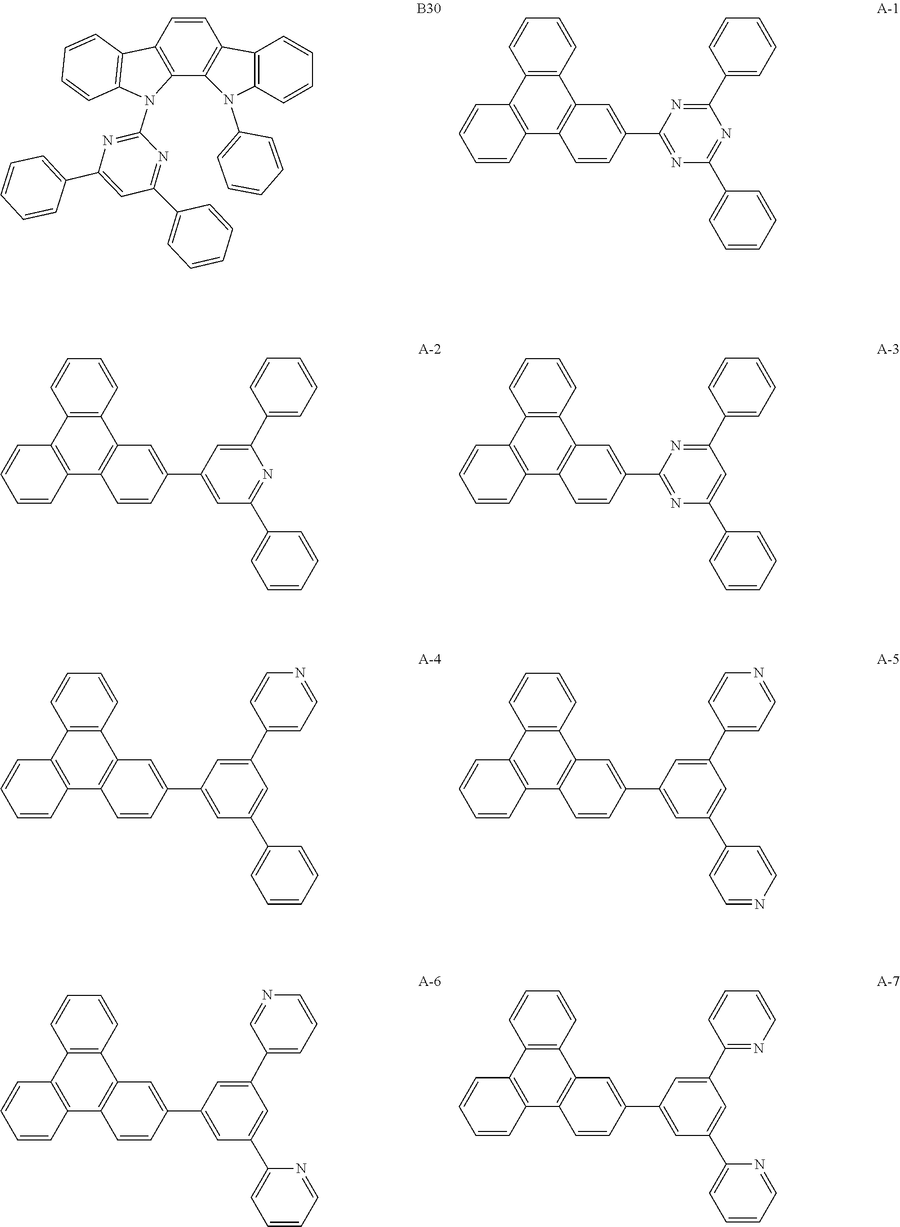

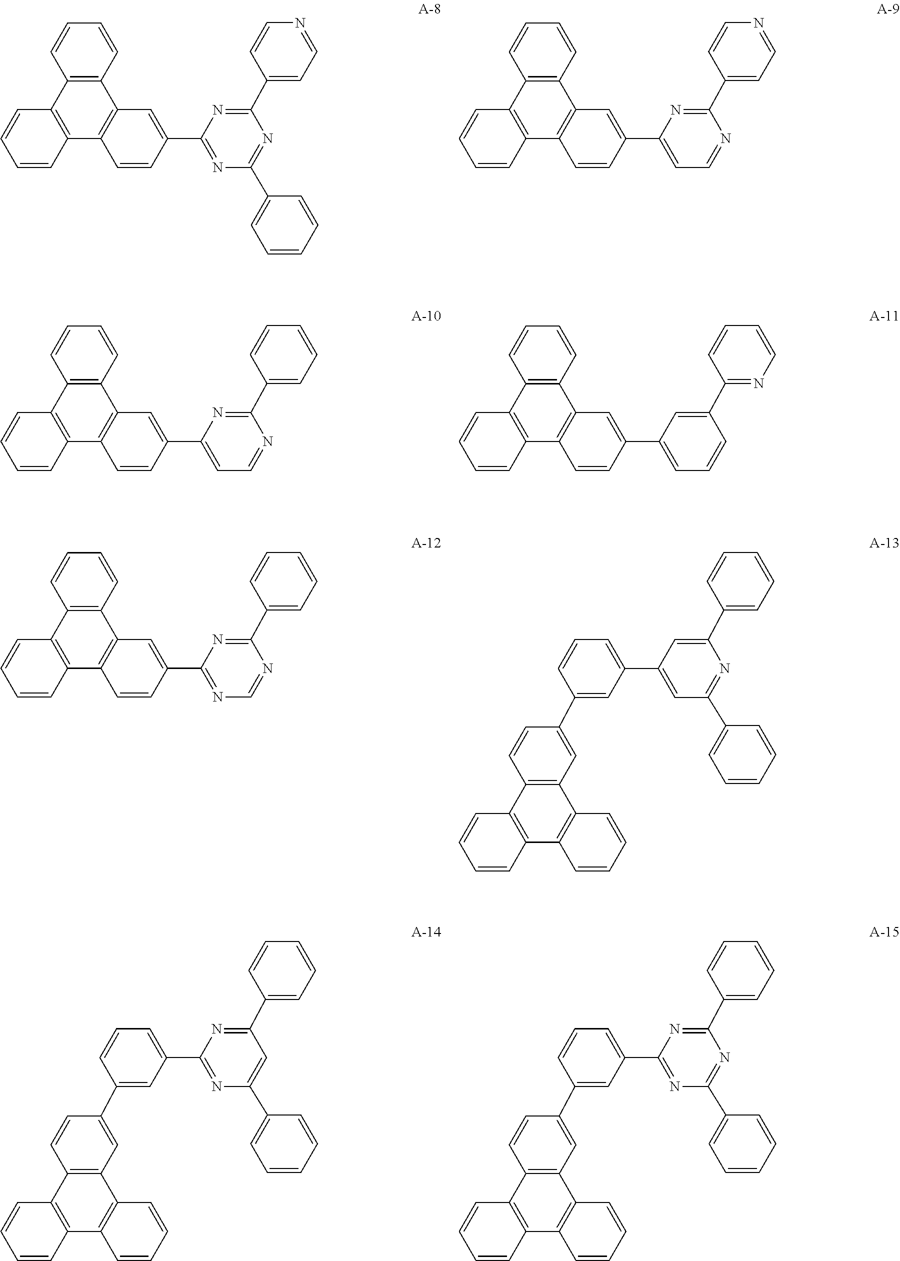

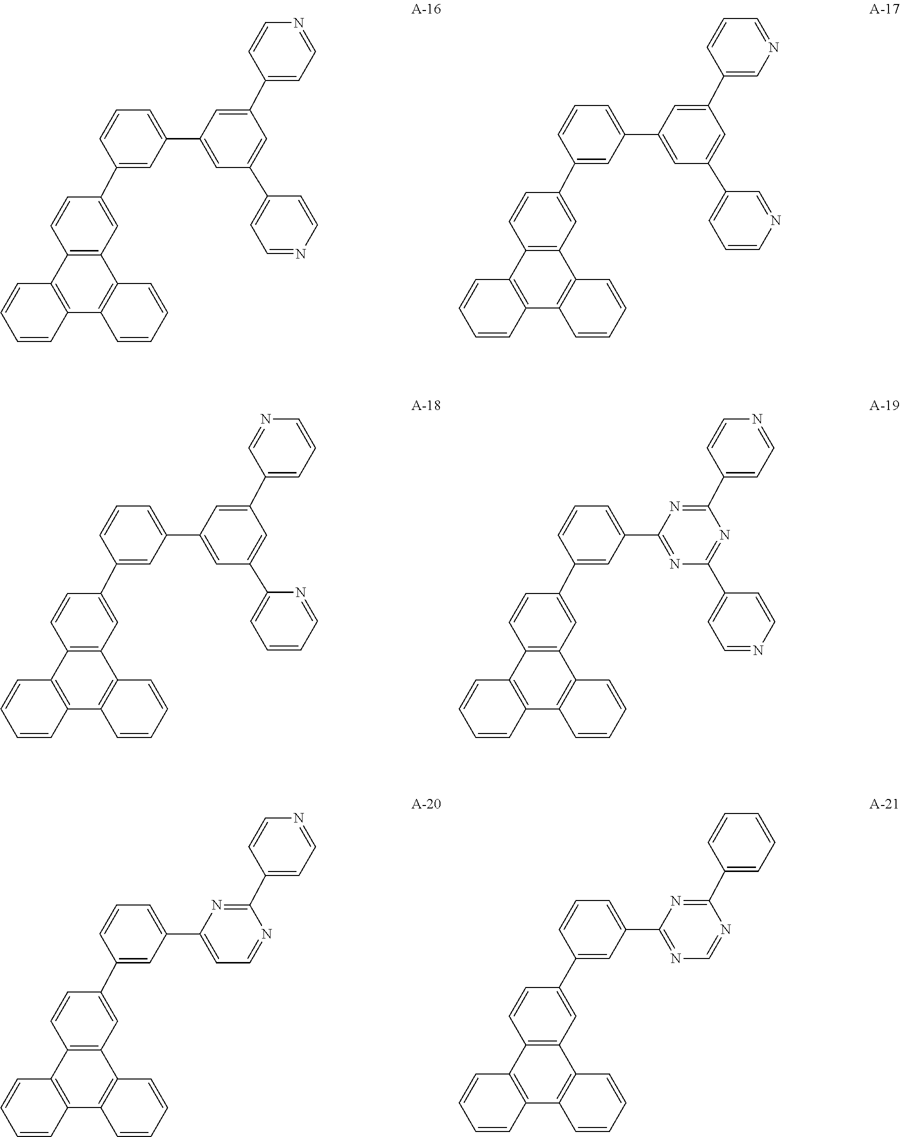 [SMILES: c1cc(-c2cc(-c3ccncc3)cc(-c3ccncc3)c2)cc(-c2ccc3c4ccccc4c4ccccc4c3c2)c1 ; c1cncc(-c2cc(-c3cccnc3)cc(-c3cccc(-c4ccc5c6ccccc6c6ccccc6c5c4)c3)c2)c1 ; c1ccc(-c2cc(-c3cccnc3)cc(-c3cccc(-c4ccc5c6ccccc6c6ccccc6c5c4)c3)c2)nc1 ; c1cc(-c2ccc3c4ccccc4c4ccccc4c3c2)cc(-c2nc(-c3ccncc3)nc(-c3ccncc3)n2)c1 ; c1cc(-c2ccc3c4ccccc4c4ccccc4c3c2)cc(-c2ccnc(-c3ccncc3)n2)c1 ; c1ccc(-c2ncnc(-c3cccc(-c4ccc5c6ccccc6c6ccccc6c5c4)c3)n2)cc1]

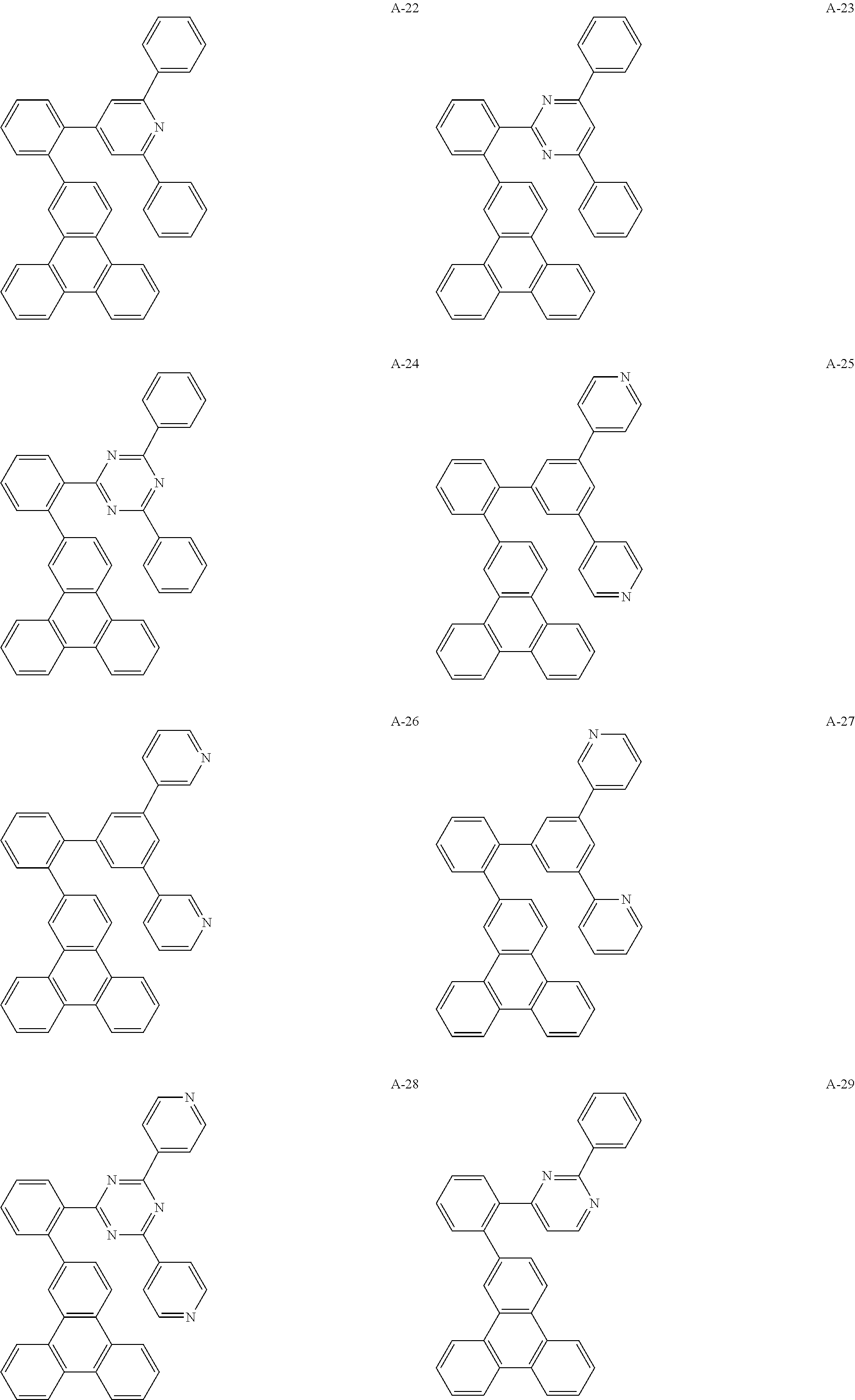

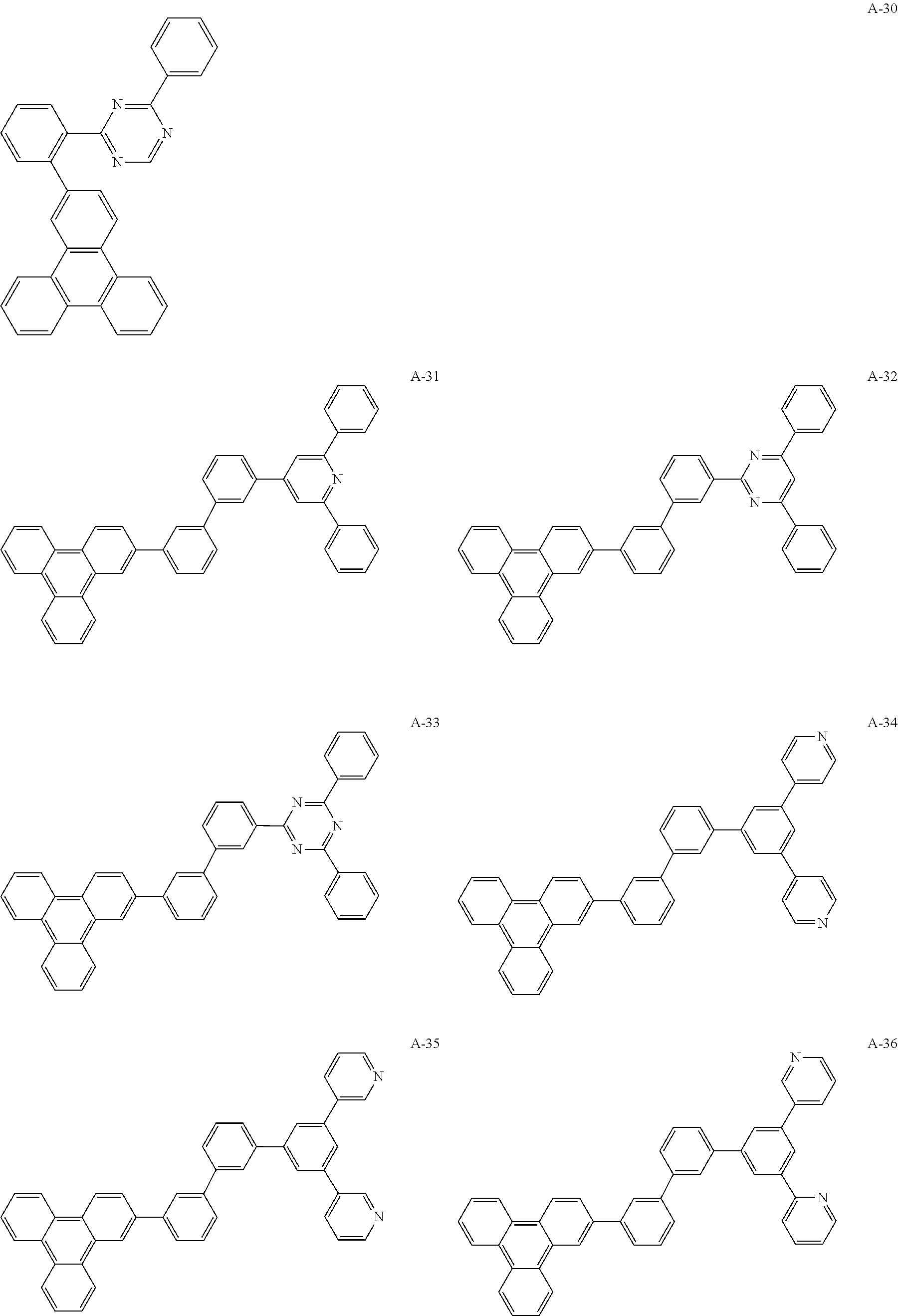

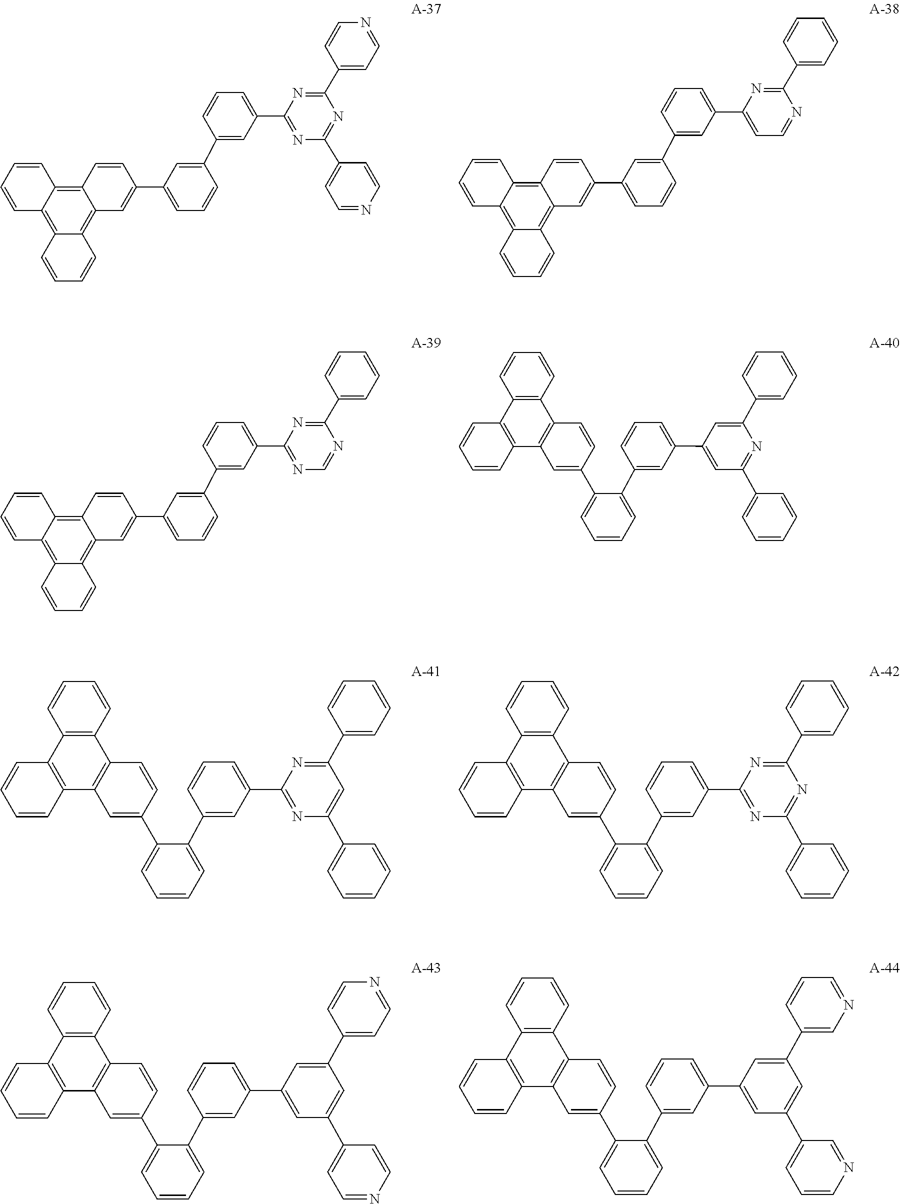

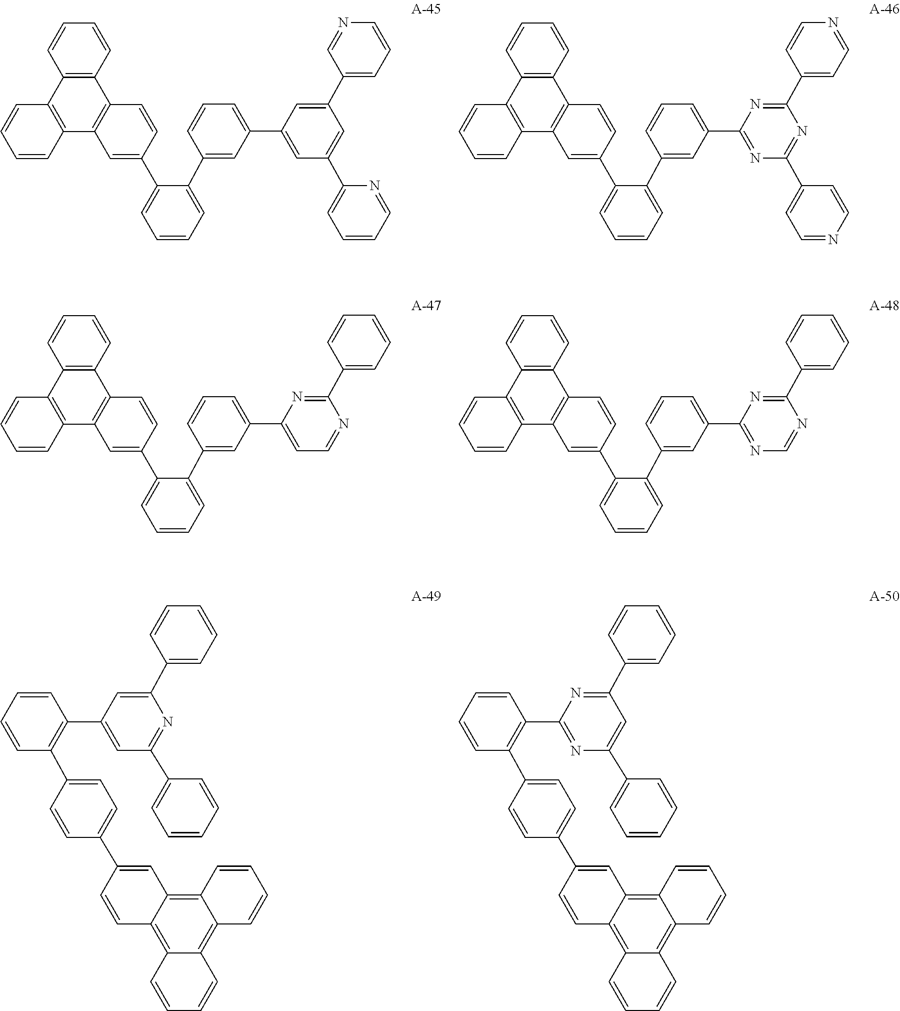 [SMILES: c1ccc(-c2cc(-c3cccnc3)cc(-c3cccc(-c4ccccc4-c4ccc5c6ccccc6c6ccccc6c5c4)c3)c2)nc1 ; c1cc(-c2nc(-c3ccncc3)nc(-c3ccncc3)n2)cc(-c2ccccc2-c2ccc3c4ccccc4c4ccccc4c3c2)c1 ; c1ccc(-c2nccc(-c3cccc(-c4ccccc4-c4ccc5c6ccccc6c6ccccc6c5c4)c3)n2)cc1 ; c1ccc(-c2ncnc(-c3cccc(-c4ccccc4-c4ccc5c6ccccc6c6ccccc6c5c4)c3)n2)cc1 ; c1ccc(-c2cc(-c3ccccc3-c3ccc(-c4ccc5c6ccccc6c6ccccc6c5c4)cc3)cc(-c3ccccc3)n2)cc1 ; c1ccc(-c2cc(-c3ccccc3)nc(-c3ccccc3-c3ccc(-c4ccc5c6ccccc6c6ccccc6c5c4)cc3)n2)cc1]

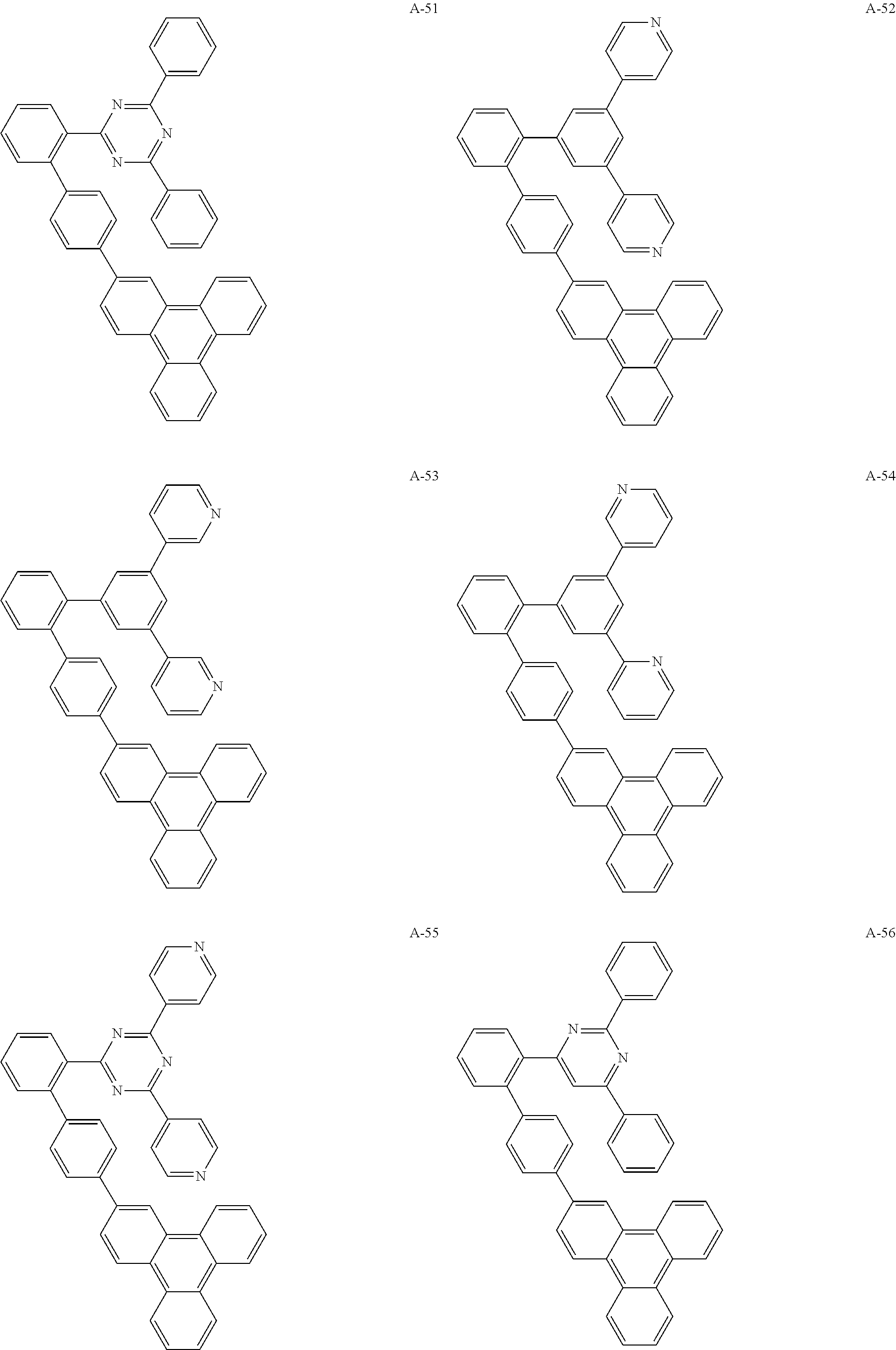 [SMILES: c1ccc(-c2nc(-c3ccccc3)nc(-c3ccccc3-c3ccc(-c4ccc5c6ccccc6c6ccccc6c5c4)cc3)n2)cc1 ; c1ccc(-c2cc(-c3ccncc3)cc(-c3ccncc3)c2)c(-c2ccc(-c3ccc4c5ccccc5c5ccccc5c4c3)cc2)c1 ; c1cncc(-c2cc(-c3cccnc3)cc(-c3ccccc3-c3ccc(-c4ccc5c6ccccc6c6ccccc6c5c4)cc3)c2)c1 ; c1ccc(-c2cc(-c3cccnc3)cc(-c3ccccc3-c3ccc(-c4ccc5c6ccccc6c6ccccc6c5c4)cc3)c2)nc1 ; c1ccc(-c2nc(-c3ccncc3)nc(-c3ccncc3)n2)c(-c2ccc(-c3ccc4c5ccccc5c5ccccc5c4c3)cc2)c1 ; c1ccc(-c2cc(-c3ccccc3-c3ccc(-c4ccc5c6ccccc6c6ccccc6c5c4)cc3)nc(-c3ccccc3)n2)cc1]

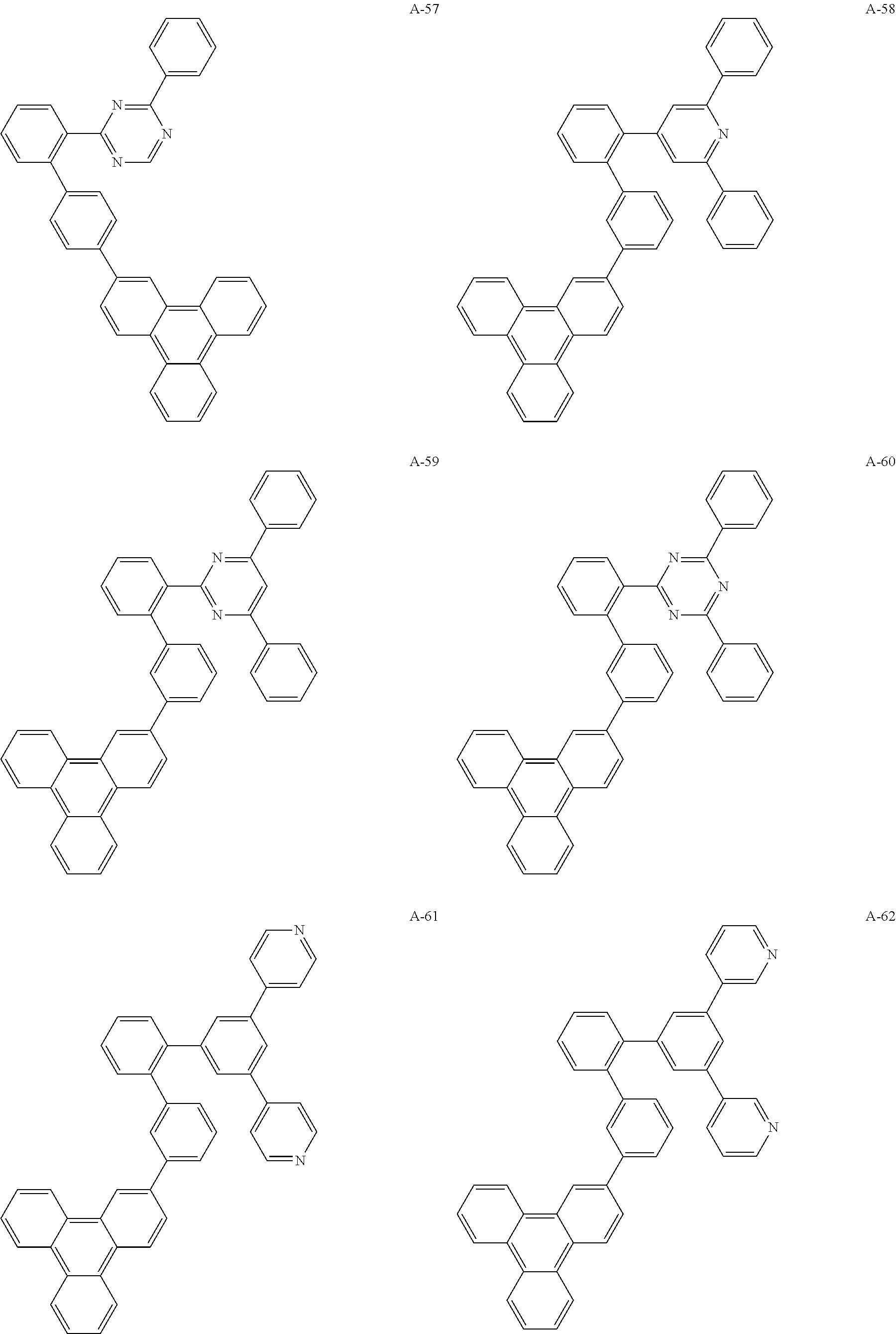 [SMILES: c1ccc(-c2ncnc(-c3ccccc3-c3ccc(-c4ccc5c6ccccc6c6ccccc6c5c4)cc3)n2)cc1 ; c1ccc(-c2cc(-c3ccccc3-c3cccc(-c4ccc5c6ccccc6c6ccccc6c5c4)c3)cc(-c3ccccc3)n2)cc1 ; c1ccc(-c2cc(-c3ccccc3)nc(-c3ccccc3-c3cccc(-c4ccc5c6ccccc6c6ccccc6c5c4)c3)n2)cc1 ; c1ccc(-c2nc(-c3ccccc3)nc(-c3ccccc3-c3cccc(-c4ccc5c6ccccc6c6ccccc6c5c4)c3)n2)cc1 ; c1cc(-c2ccc3c4ccccc4c4ccccc4c3c2)cc(-c2ccccc2-c2cc(-c3ccncc3)cc(-c3ccncc3)c2)c1 ; c1cncc(-c2cc(-c3cccnc3)cc(-c3ccccc3-c3cccc(-c4ccc5c6ccccc6c6ccccc6c5c4)c3)c2)c1]

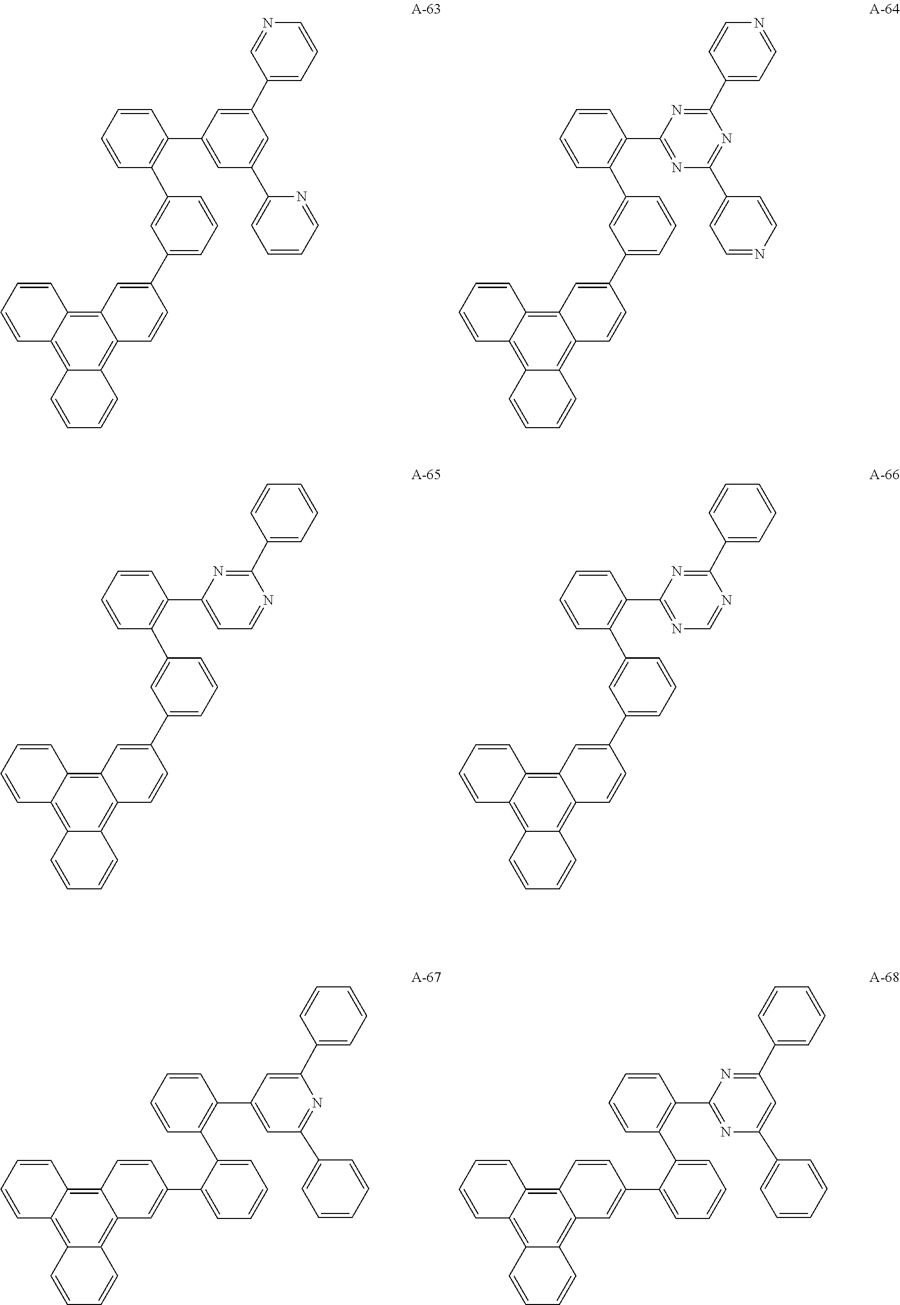 [SMILES: c1ccc(-c2cc(-c3cccnc3)cc(-c3ccccc3-c3cccc(-c4ccc5c6ccccc6c6ccccc6c5c4)c3)c2)nc1 ; c1cc(-c2ccc3c4ccccc4c4ccccc4c3c2)cc(-c2ccccc2-c2nc(-c3ccncc3)nc(-c3ccncc3)n2)c1 ; c1ccc(-c2nccc(-c3ccccc3-c3cccc(-c4ccc5c6ccccc6c6ccccc6c5c4)c3)n2)cc1 ; c1ccc(-c2ncnc(-c3ccccc3-c3cccc(-c4ccc5c6ccccc6c6ccccc6c5c4)c3)n2)cc1 ; c1ccc(-c2cc(-c3ccccc3-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccccc3-c3ccc4c5ccccc5c5ccccc5c4c3)n2)cc1]

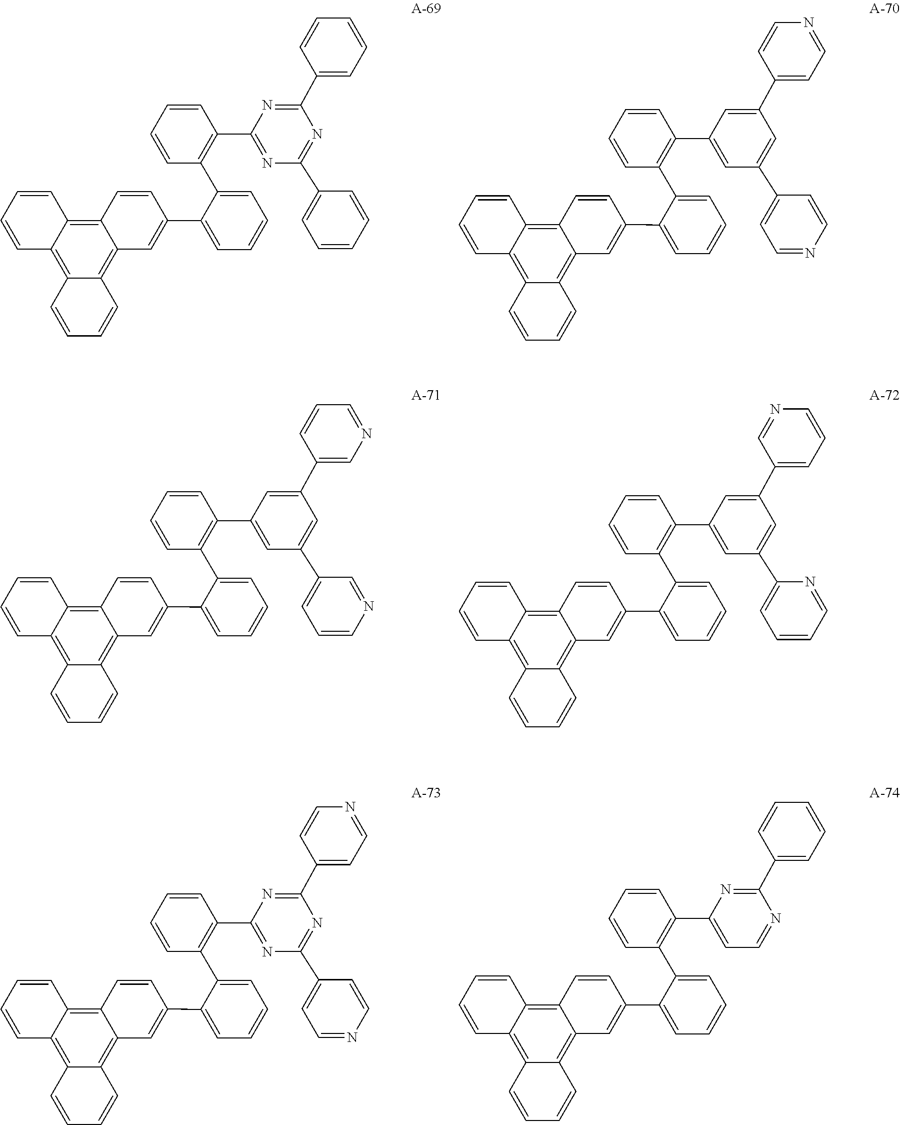 [SMILES: c1ccc(-c2nc(-c3ccccc3)nc(-c3ccccc3-c3ccccc3-c3ccc4c5ccccc5c5ccccc5c4c3)n2)cc1 ; c1ccc(-c2ccccc2-c2ccc3c4ccccc4c4ccccc4c3c2)c(-c2cc(-c3ccncc3)cc(-c3ccncc3)c2)c1 ; c1cncc(-c2cc(-c3cccnc3)cc(-c3ccccc3-c3ccccc3-c3ccc4c5ccccc5c5ccccc5c4c3)c2)c1 ; c1ccc(-c2cc(-c3cccnc3)cc(-c3ccccc3-c3ccccc3-c3ccc4c5ccccc5c5ccccc5c4c3)c2)nc1 ; c1ccc(-c2ccccc2-c2nc(-c3ccncc3)nc(-c3ccncc3)n2)c(-c2ccc3c4ccccc4c4ccccc4c3c2)c1 ; c1ccc(-c2nccc(-c3ccccc3-c3ccccc3-c3ccc4c5ccccc5c5ccccc5c4c3)n2)cc1]

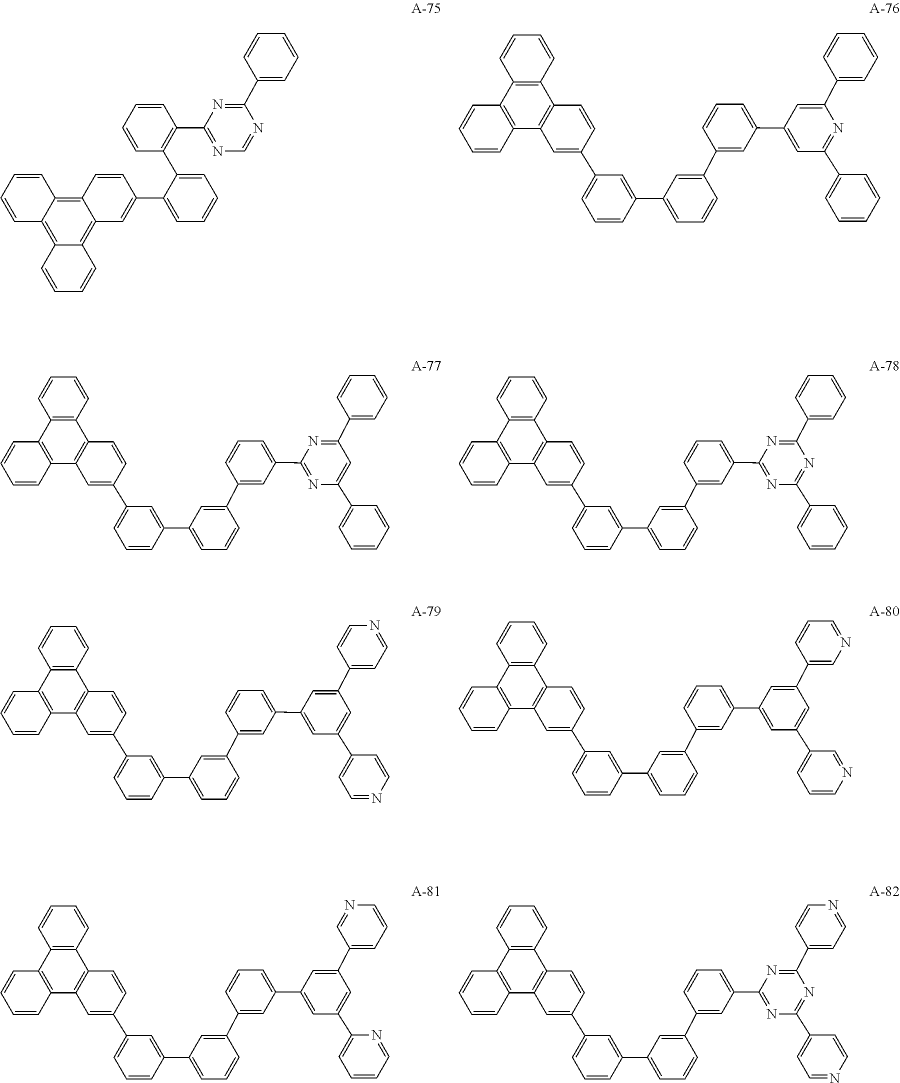

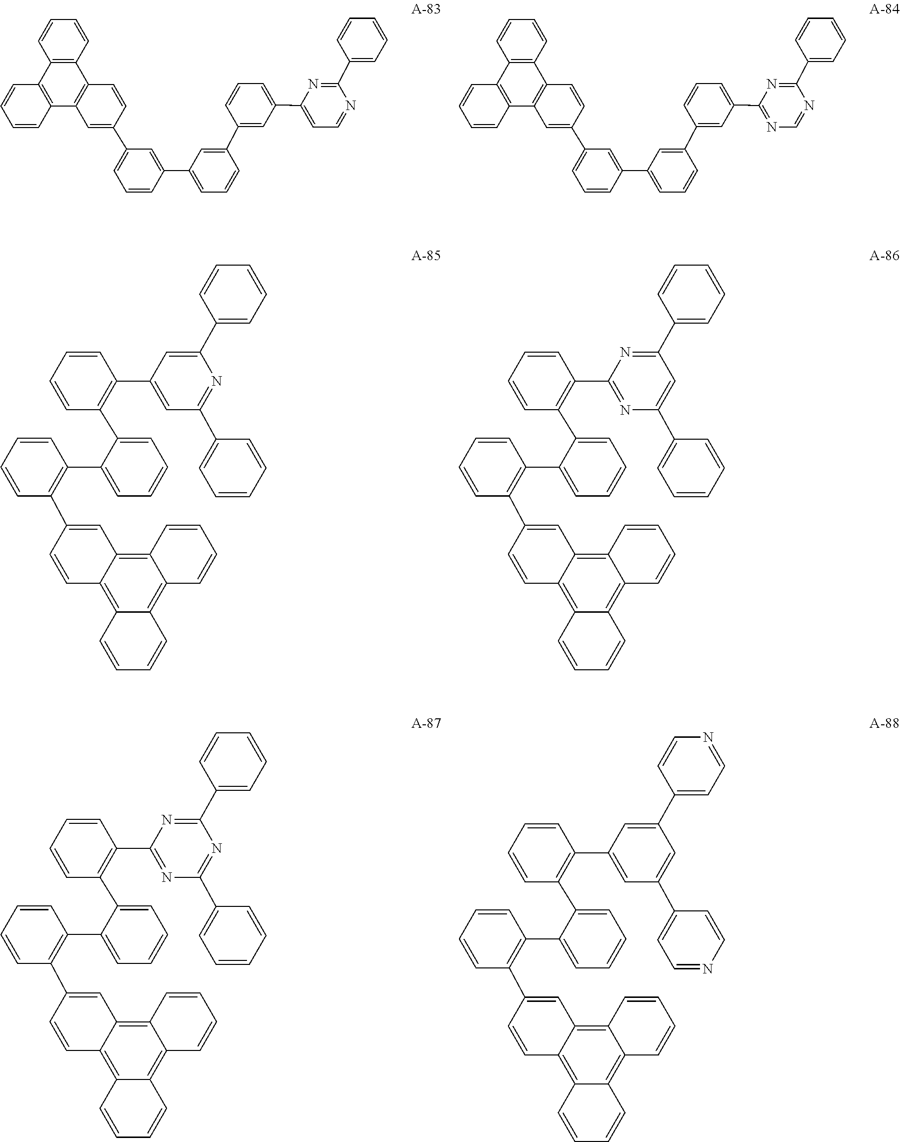

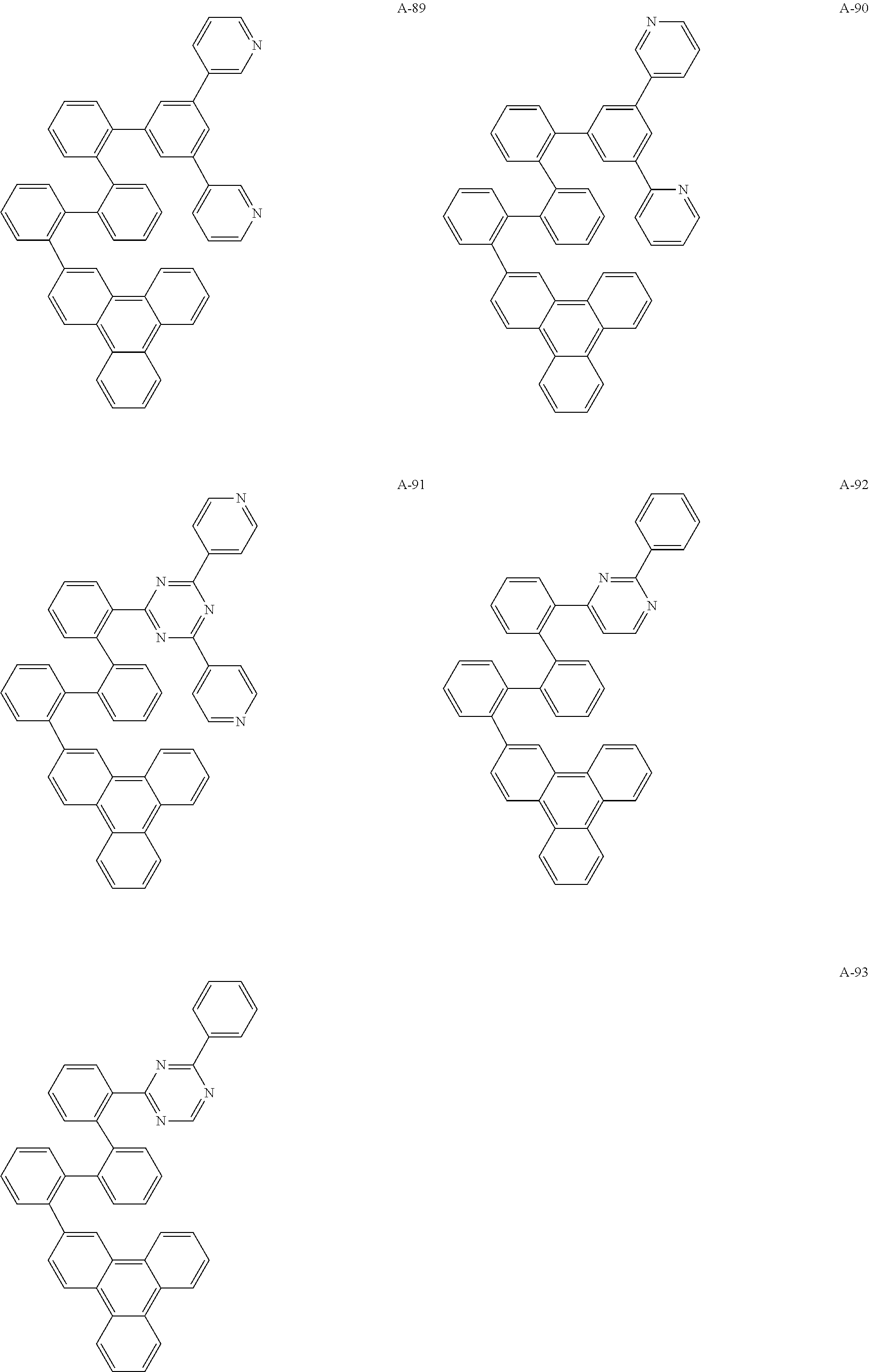 [SMILES: c1cncc(-c2cc(-c3cccnc3)cc(-c3ccccc3-c3ccccc3-c3ccccc3-c3ccc4c5ccccc5c5ccccc5c4c3)c2)c1 ; c1ccc(-c2cc(-c3cccnc3)cc(-c3ccccc3-c3ccccc3-c3ccccc3-c3ccc4c5ccccc5c5ccccc5c4c3)c2)nc1 ; c1ccc(-c2ccccc2-c2ccccc2-c2nc(-c3ccncc3)nc(-c3ccncc3)n2)c(-c2ccc3c4ccccc4c4ccccc4c3c2)c1 ; c1ccc(-c2nccc(-c3ccccc3-c3ccccc3-c3ccccc3-c3ccc4c5ccccc5c5ccccc5c4c3)n2)cc1 ; c1ccc(-c2ncnc(-c3ccccc3-c3ccccc3-c3ccccc3-c3ccc4c5ccccc5c5ccccc5c4c3)n2)cc1]

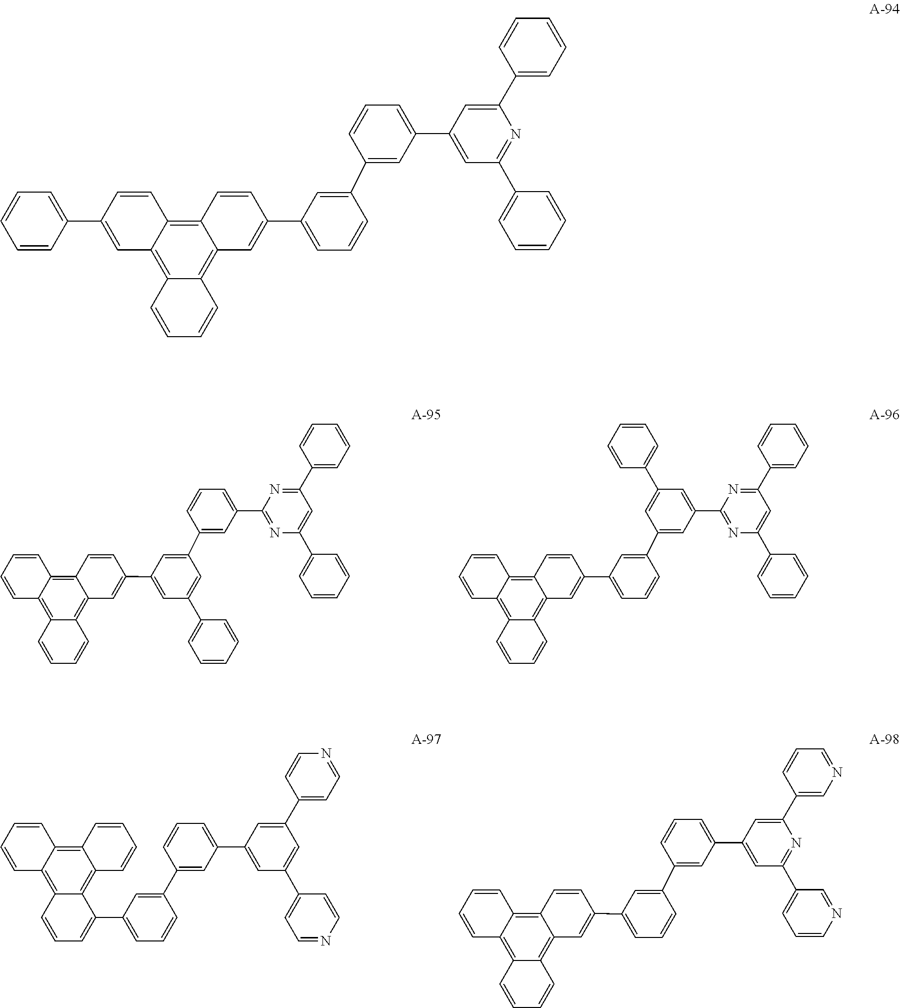 [SMILES: c1ccc(-c2ccc3c4ccc(-c5cccc(-c6cccc(-c7cc(-c8ccccc8)nc(-c8ccccc8)c7)c6)c5)cc4c4ccccc4c3c2)cc1 ; c1ccc(-c2cc(-c3cccc(-c4nc(-c5ccccc5)cc(-c5ccccc5)n4)c3)cc(-c3ccc4c5ccccc5c5ccccc5c4c3)c2)cc1 ; c1ccc(-c2cc(-c3cccc(-c4ccc5c6ccccc6c6ccccc6c5c4)c3)cc(-c3nc(-c4ccccc4)cc(-c4ccccc4)n3)c2)cc1 ; c1cc(-c2cc(-c3ccncc3)cc(-c3ccncc3)c2)cc(-c2cccc(-c3cccc4c5ccccc5c5ccccc5c34)c2)c1 ; c1cc(-c2cccc(-c3ccc4c5ccccc5c5ccccc5c4c3)c2)cc(-c2cc(-c3cccnc3)nc(-c3cccnc3)c2)c1]

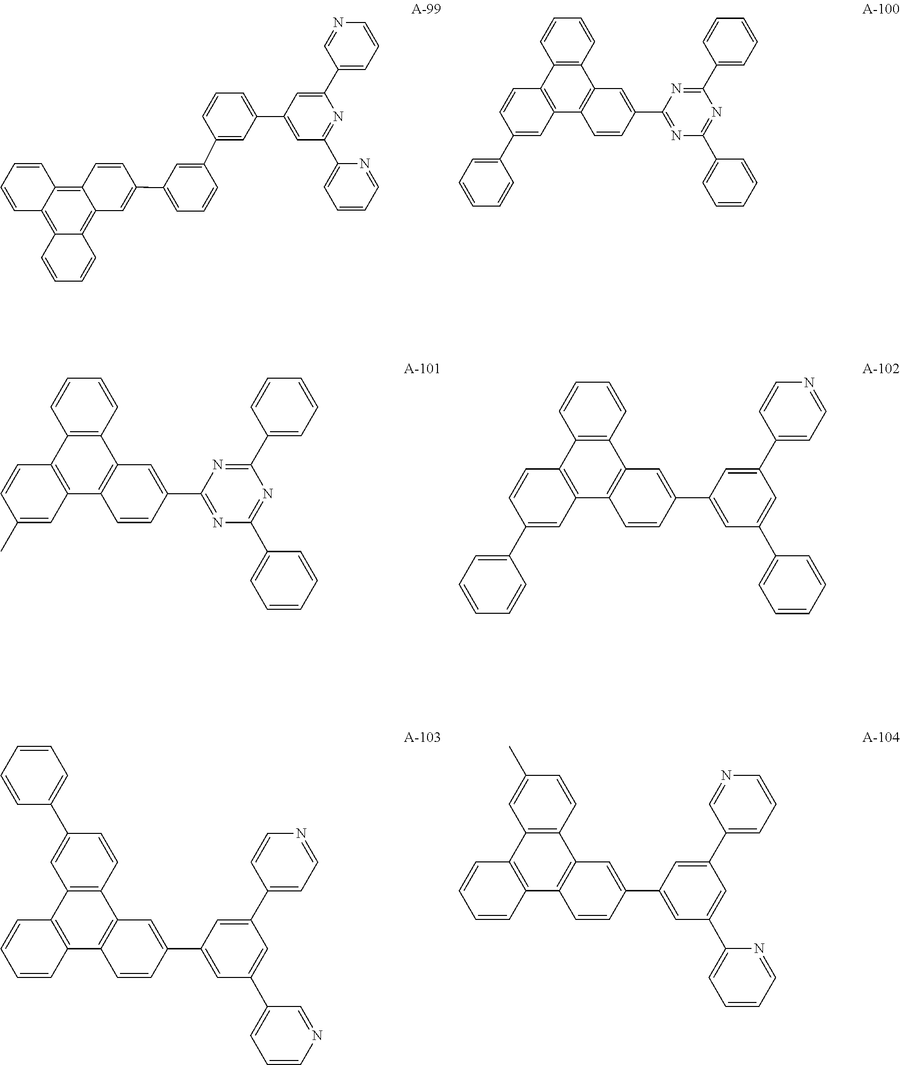 [SMILES: c1ccc(-c2cc(-c3cccc(-c4cccc(-c5ccc6c7ccccc7c7ccccc7c6c5)c4)c3)cc(-c3cccnc3)n2)nc1 ; c1ccc(-c2ccc3c4ccccc4c4cc(-c5nc(-c6ccccc6)nc(-c6ccccc6)n5)ccc4c3c2)cc1 ; Cc1ccc2c3ccccc3c3cc(-c4nc(-c5ccccc5)nc(-c5ccccc5)n4)ccc3c2c1 ; c1ccc(-c2cc(-c3ccncc3)cc(-c3ccc4c5cc(-c6ccccc6)ccc5c5ccccc5c4c3)c2)cc1 ; c1ccc(-c2ccc3c(c2)c2ccccc2c2ccc(-c4cc(-c5ccncc5)cc(-c5cccnc5)c4)cc23)cc1 ; Cc1ccc2c(c1)c1ccccc1c1ccc(-c3cc(-c4cccnc4)cc(-c4ccccn4)c3)cc12]

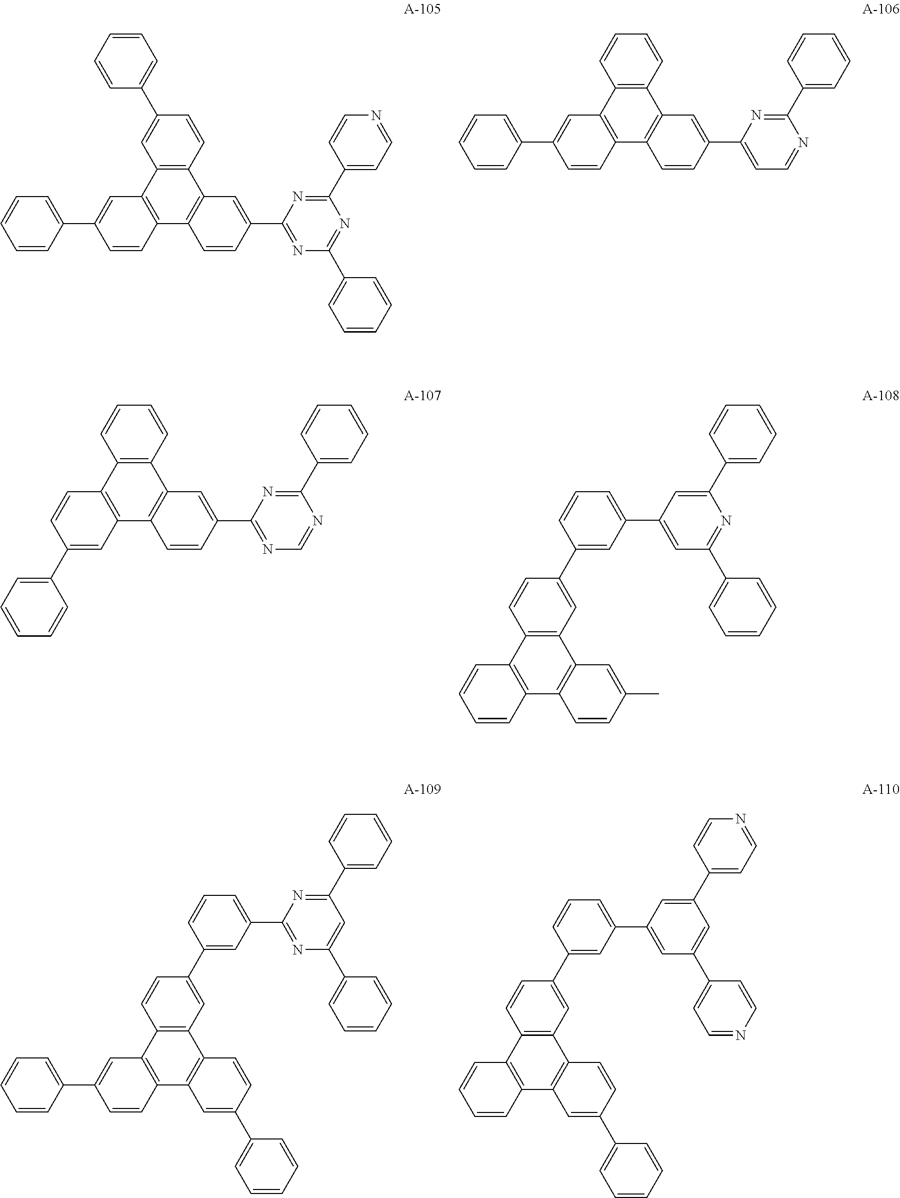 [SMILES: c1ccc(-c2ccc3c4ccc(-c5nc(-c6ccccc6)nc(-c6ccncc6)n5)cc4c4ccc(-c5ccccc5)cc4c3c2)cc1 ; c1ccc(-c2ccc3c4ccc(-c5ccnc(-c6ccccc6)n5)cc4c4ccccc4c3c2)cc1 ; c1ccc(-c2ccc3c4ccccc4c4cc(-c5ncnc(-c6ccccc6)n5)ccc4c3c2)cc1 ; Cc1ccc2c3ccccc3c3ccc(-c4cccc(-c5cc(-c6ccccc6)nc(-c6ccccc6)c5)c4)cc3c2c1 ; c1ccc(-c2ccc3c(c2)c2ccc(-c4cccc(-c5nc(-c6ccccc6)cc(-c6ccccc6)n5)c4)cc2c2ccc(-c4ccccc4)cc32)cc1 ; c1ccc(-c2ccc3c(c2)c2ccccc2c2ccc(-c4cccc(-c5cc(-c6ccncc6)cc(-c6ccncc6)c5)c4)cc23)cc1]

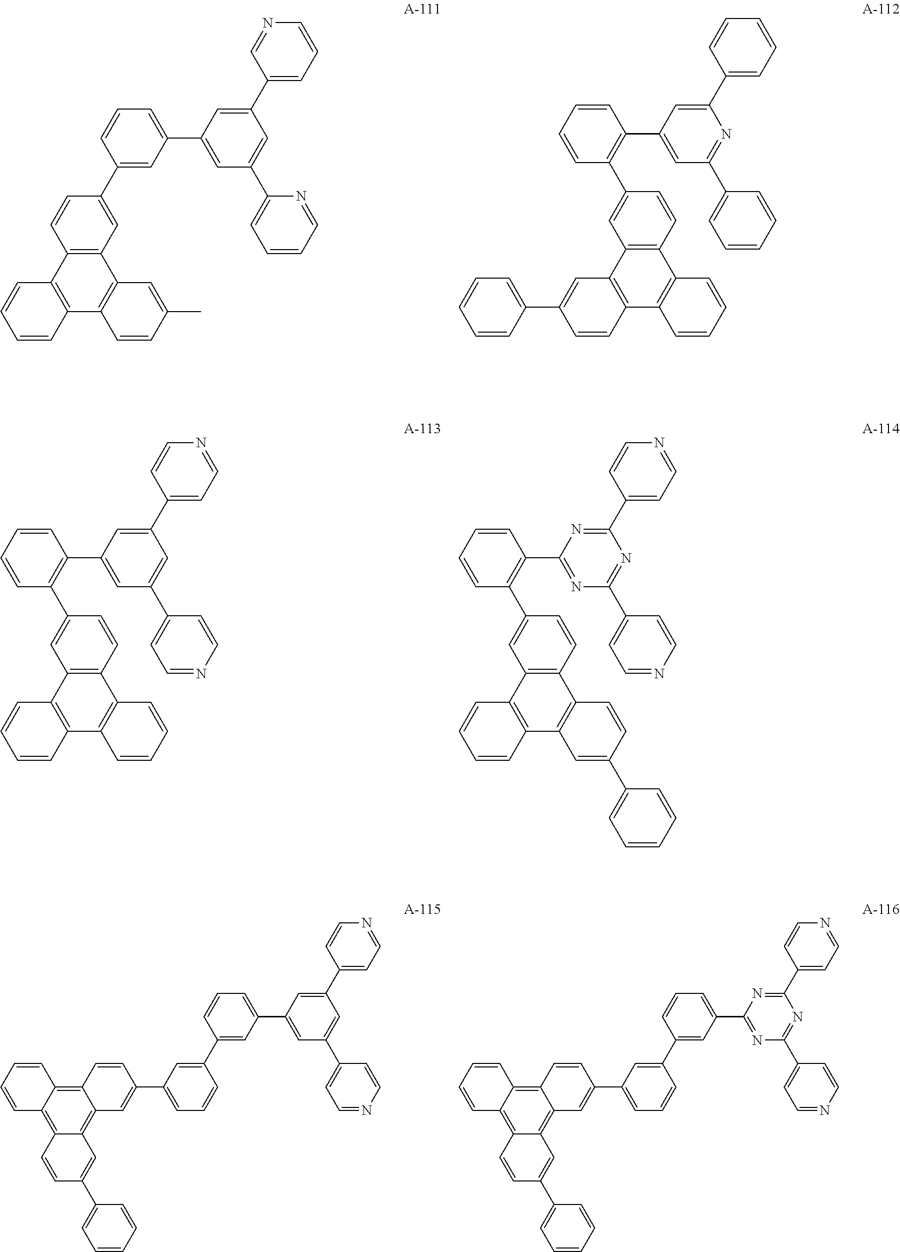 [SMILES: Cc1ccc2c3ccccc3c3ccc(-c4cccc(-c5cc(-c6cccnc6)cc(-c6ccccn6)c5)c4)cc3c2c1 ; c1ccc(-c2ccc3c4ccccc4c4ccc(-c5ccccc5-c5cc(-c6ccccc6)nc(-c6ccccc6)c5)cc4c3c2)cc1 ; c1ccc(-c2ccc3c4ccccc4c4ccccc4c3c2)c(-c2cc(-c3ccncc3)cc(-c3ccncc3)c2)c1 ; c1ccc(-c2ccc3c4ccc(-c5ccccc5-c5nc(-c6ccncc6)nc(-c6ccncc6)n5)cc4c4ccccc4c3c2)cc1 ; c1ccc(-c2ccc3c4ccccc4c4ccc(-c5cccc(-c6cccc(-c7cc(-c8ccncc8)cc(-c8ccncc8)c7)c6)c5)cc4c3c2)cc1 ; c1ccc(-c2ccc3c4ccccc4c4ccc(-c5cccc(-c6cccc(-c7nc(-c8ccncc8)nc(-c8ccncc8)n7)c6)c5)cc4c3c2)cc1]

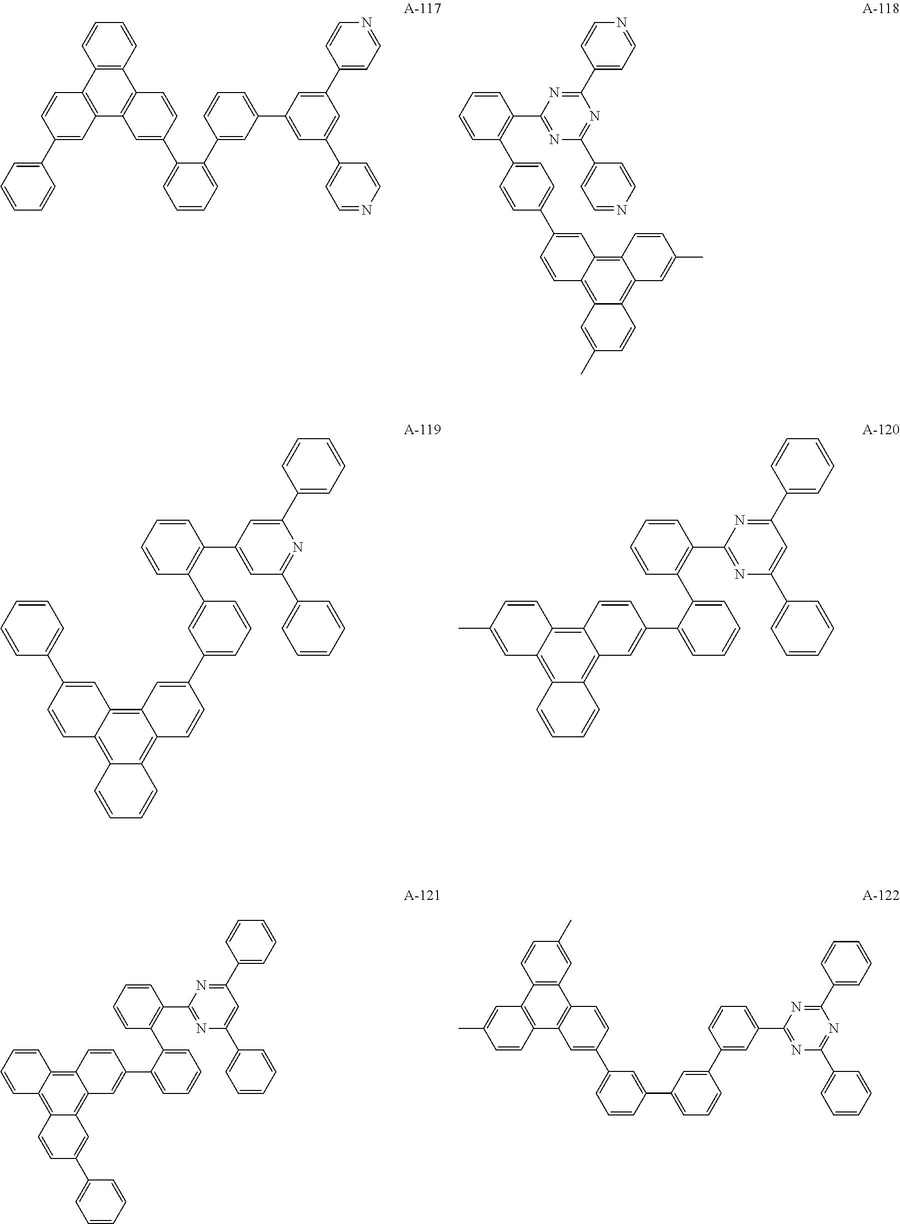

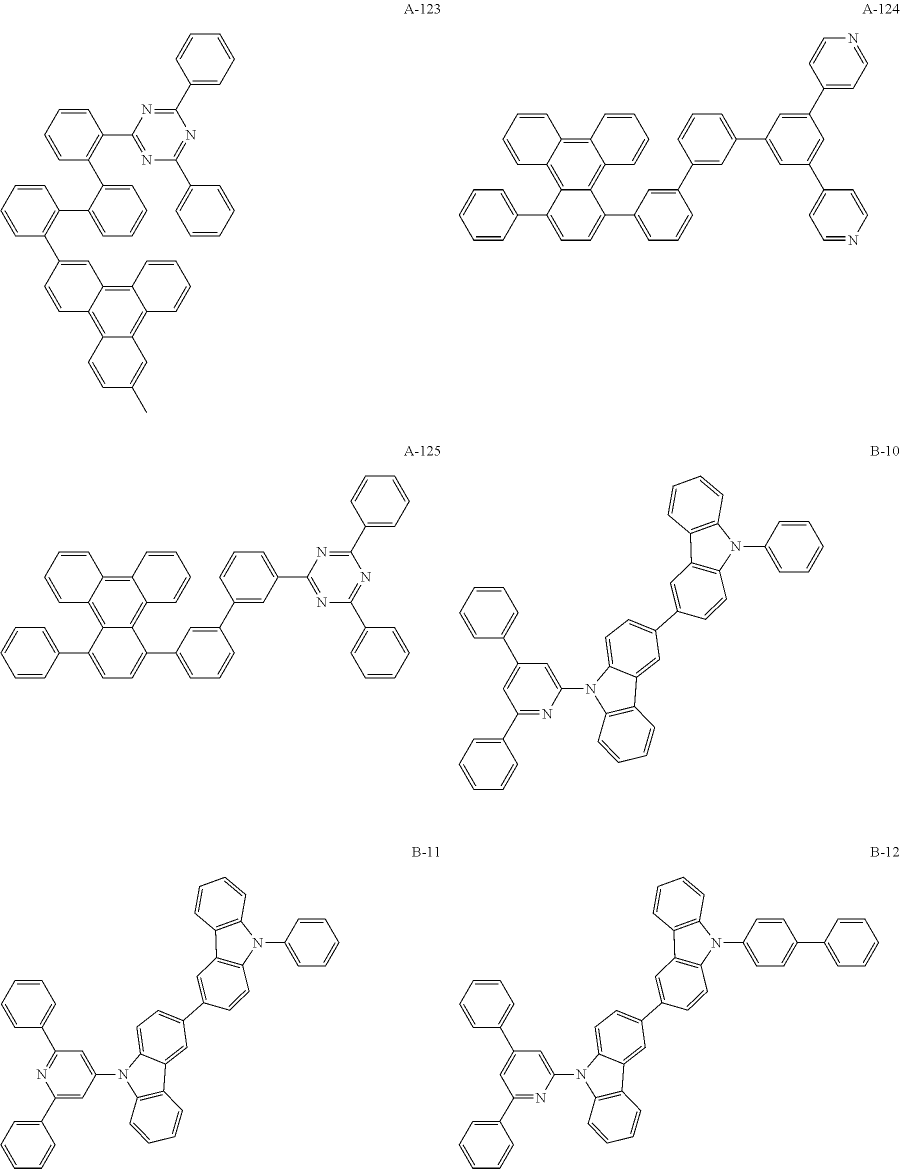

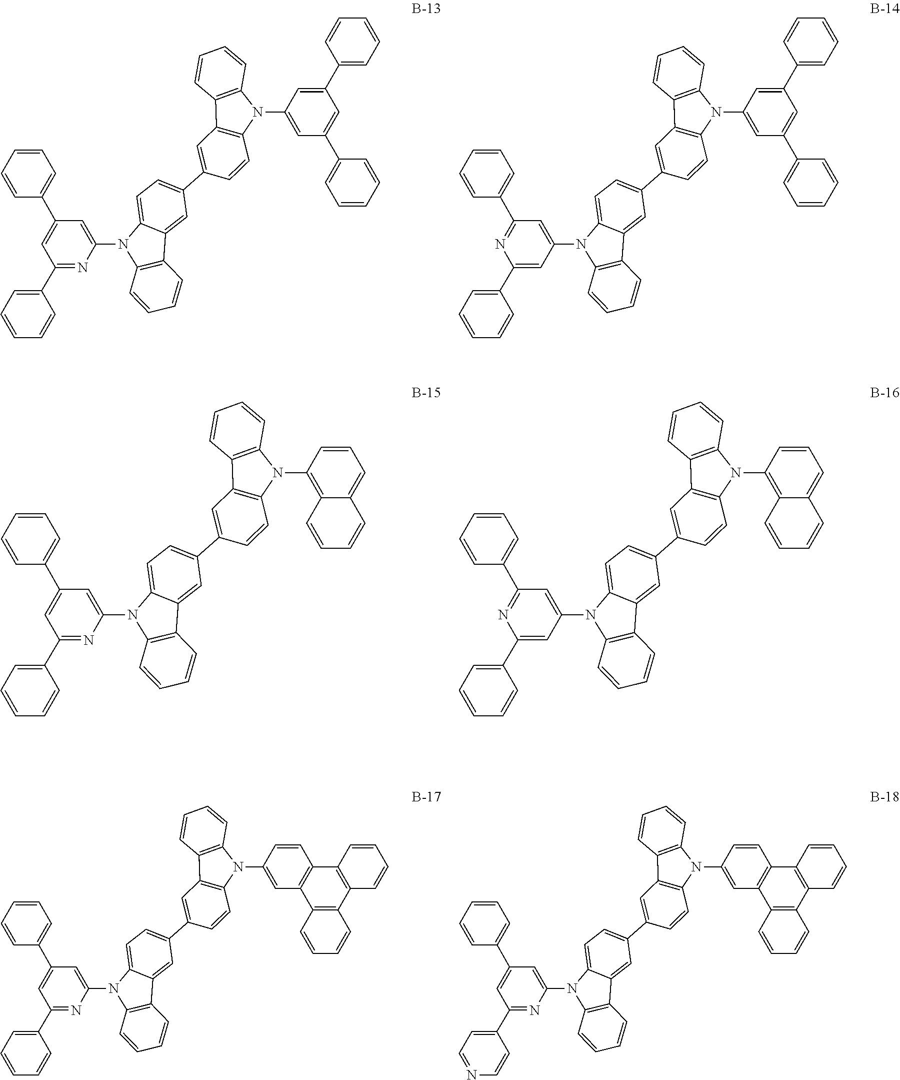

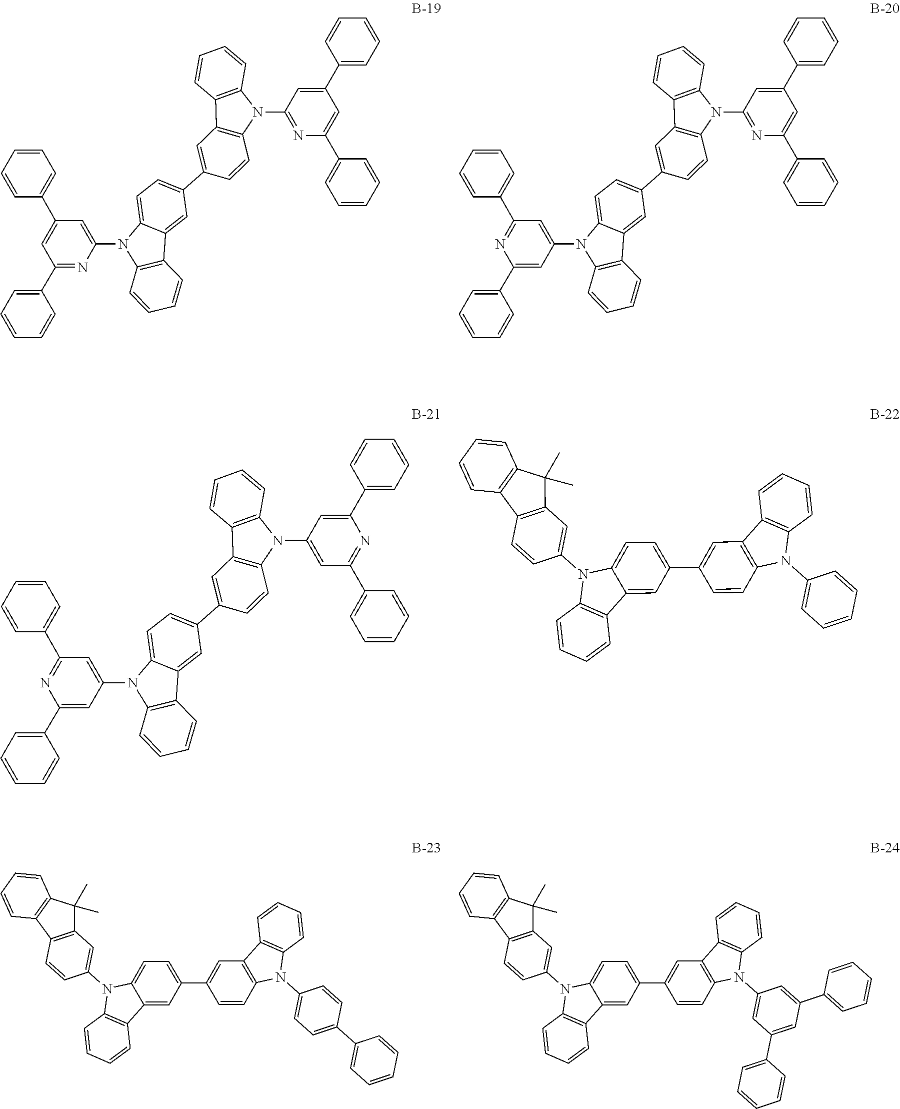

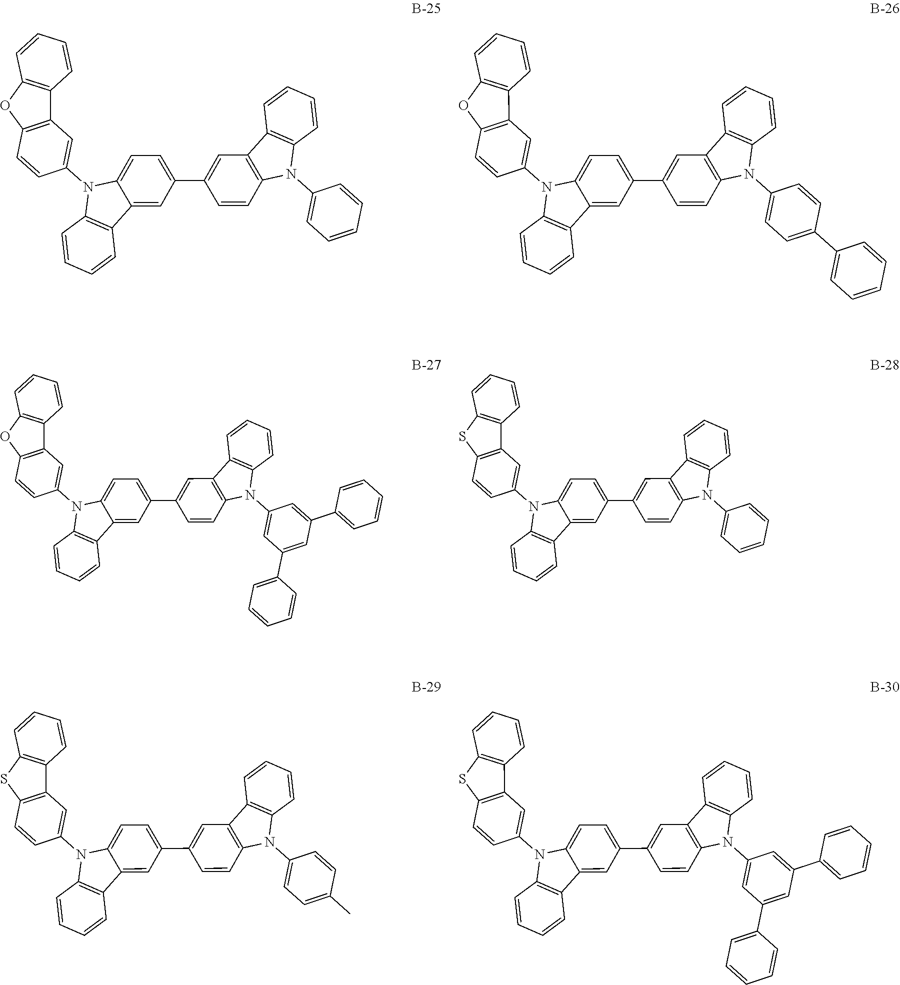

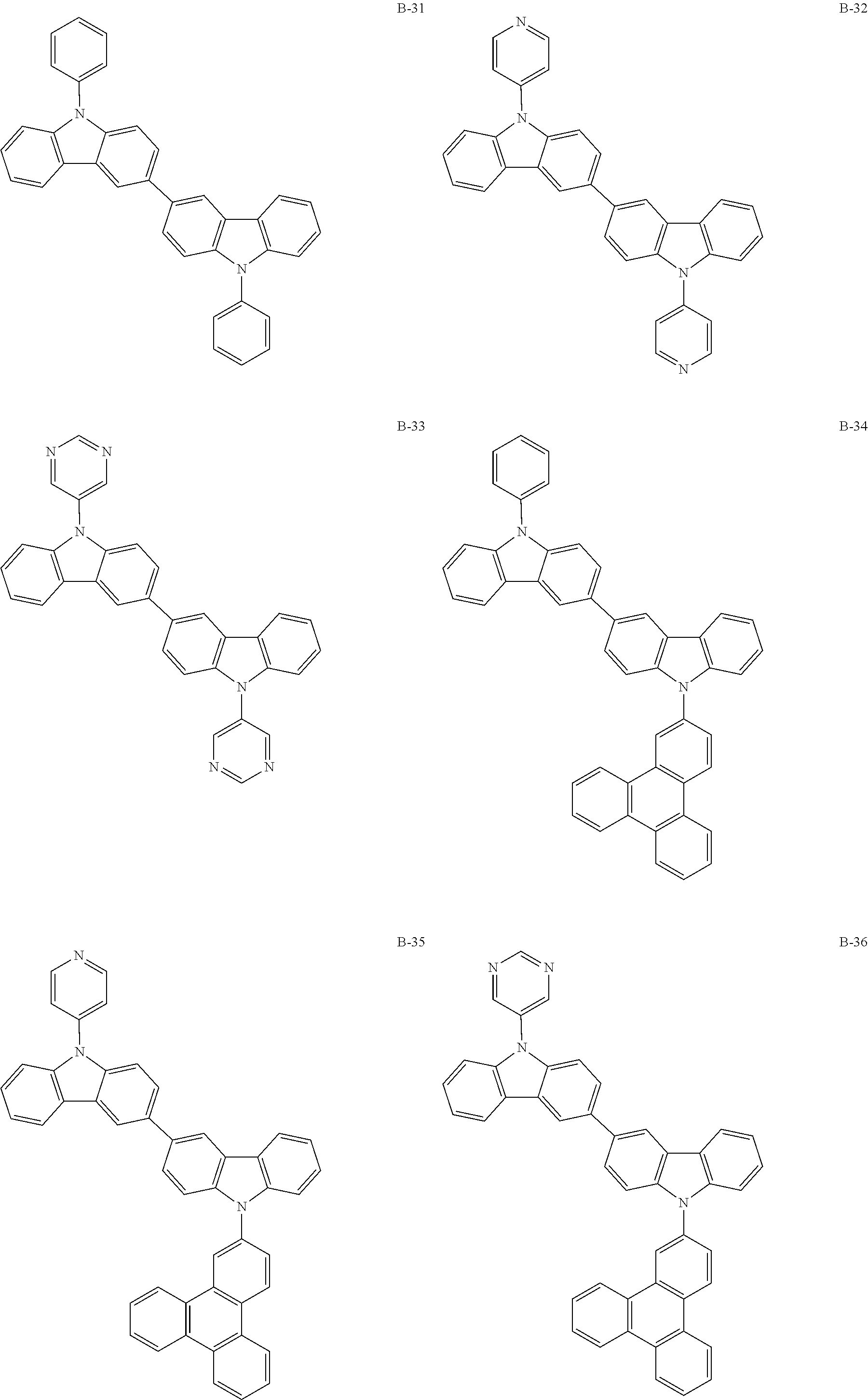 [SMILES: c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1 ; c1ccc2c(c1)c1ccccc1c1cc(-n3c4ccccc4c4cc(-c5ccc6c(c5)c5ccccc5n6-c5ccncc5)ccc43)ccc21 ; c1ccc2c(c1)c1ccccc1c1cc(-n3c4ccccc4c4cc(-c5ccc6c(c5)c5ccccc5n6-c5cncnc5)ccc43)ccc21]

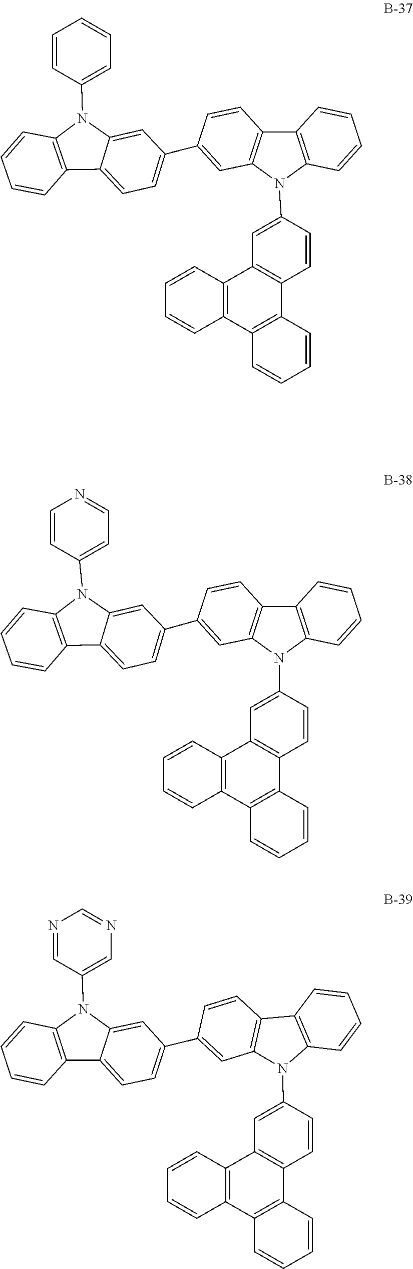 [SMILES: c1ccc(-n2c3ccccc3c3ccc(-c4ccc5c6ccccc6n(-c6ccc7c8ccccc8c8ccccc8c7c6)c5c4)cc32)cc1 ; c1ccc2c(c1)c1ccccc1c1cc(-n3c4ccccc4c4ccc(-c5ccc6c7ccccc7n(-c7ccncc7)c6c5)cc43)ccc21 ; c1ccc2c(c1)c1ccccc1c1cc(-n3c4ccccc4c4ccc(-c5ccc6c7ccccc7n(-c7cncnc7)c6c5)cc43)ccc21]

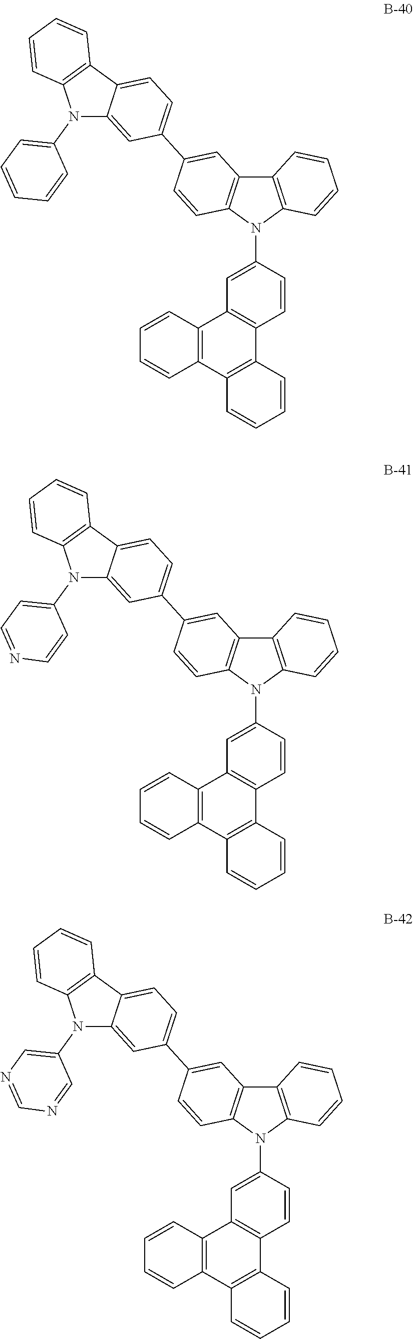 [SMILES: c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)c1ccccc1c1cc(-n3c4ccccc4c4cc(-c5ccc6c7ccccc7n(-c7ccncc7)c6c5)ccc43)ccc21 ; c1ccc2c(c1)c1ccccc1c1cc(-n3c4ccccc4c4cc(-c5ccc6c7ccccc7n(-c7cncnc7)c6c5)ccc43)ccc21]

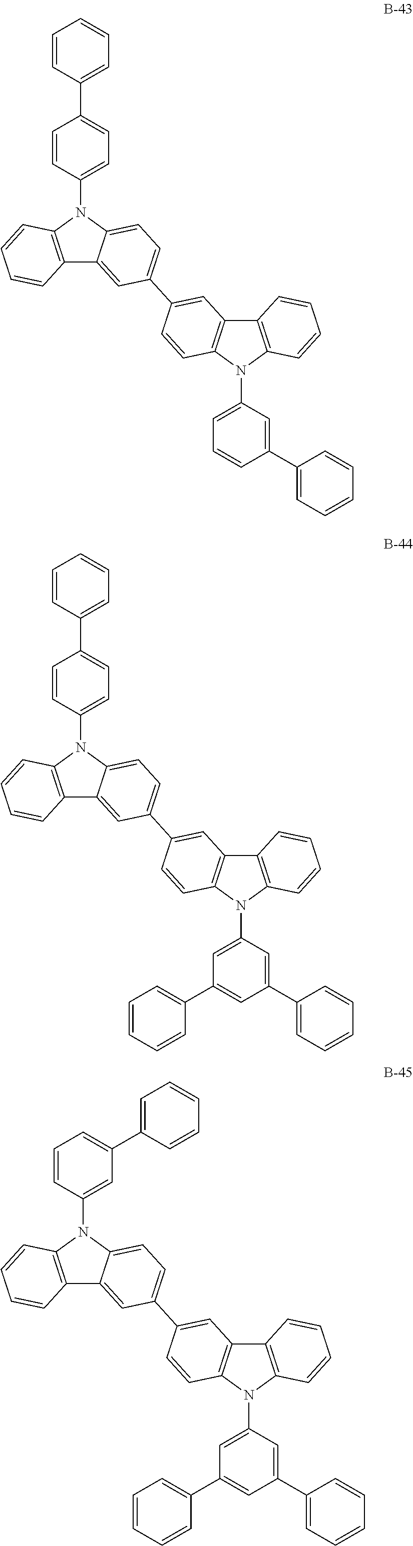

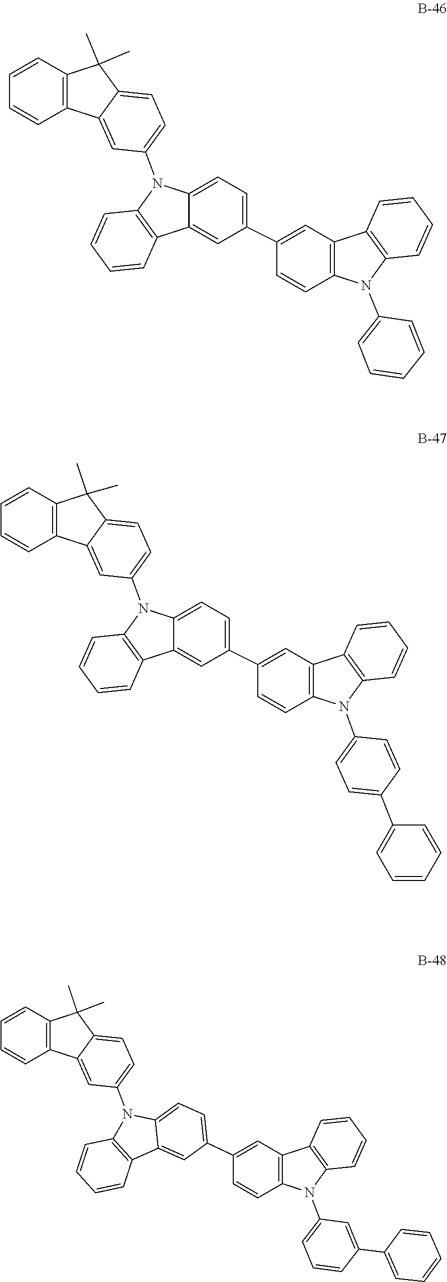 [SMILES: CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccc(-c6ccccc6)cc5)ccc43)ccc21 ; CC1(C)c2ccccc2-c2cc(-n3c4ccccc4c4cc(-c5ccc6c(c5)c5ccccc5n6-c5cccc(-c6ccccc6)c5)ccc43)ccc21]

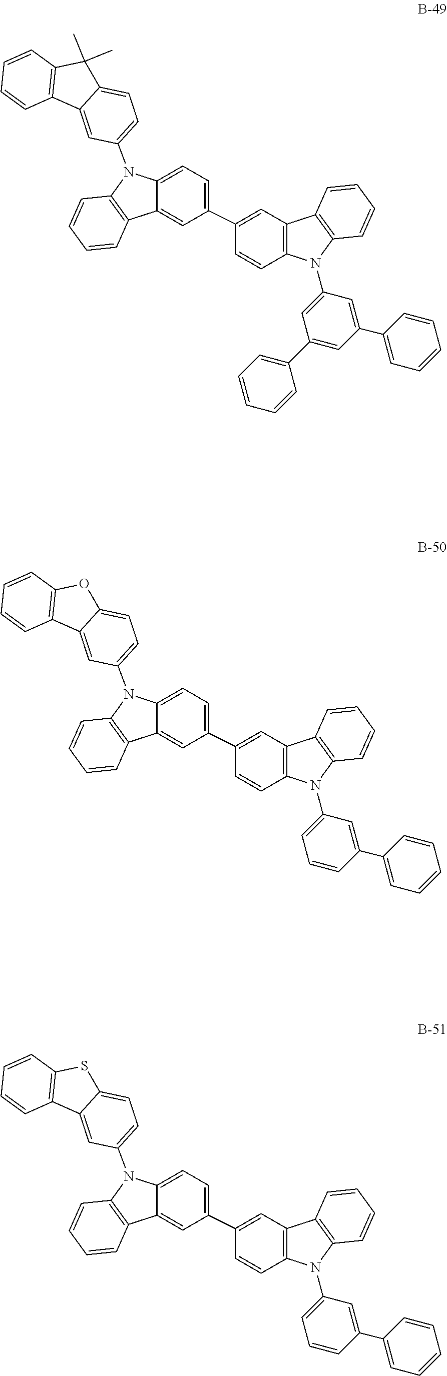 [SMILES: CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21 ; c1ccc(-c2cccc(-n3c4ccccc4c4cc(-c5ccc6c(c5)c5ccccc5n6-c5ccc6oc7ccccc7c6c5)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1]

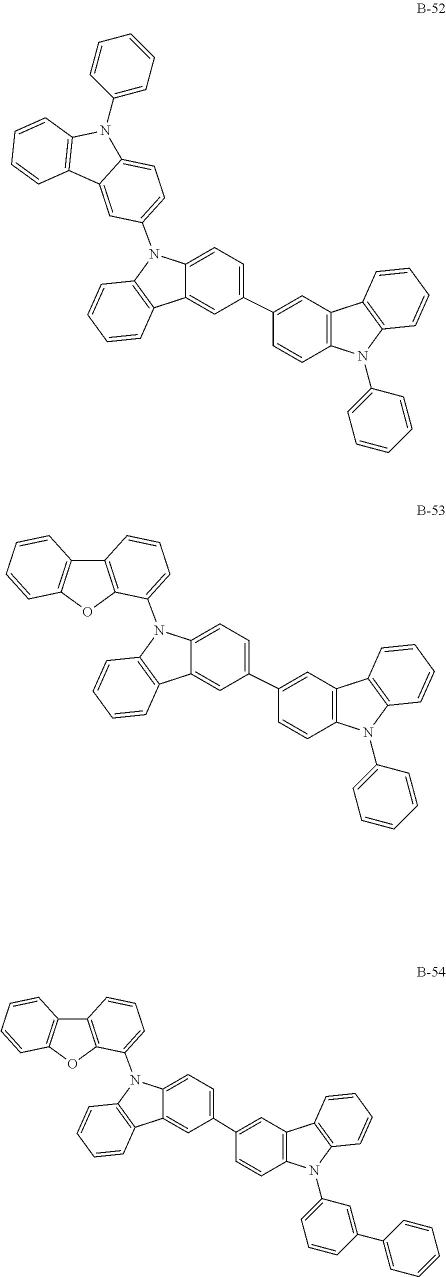 [SMILES: c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5c4oc4ccccc45)ccc32)cc1 ; c1ccc(-c2cccc(-n3c4ccccc4c4cc(-c5ccc6c(c5)c5ccccc5n6-c5cccc6c5oc5ccccc56)ccc43)c2)cc1]

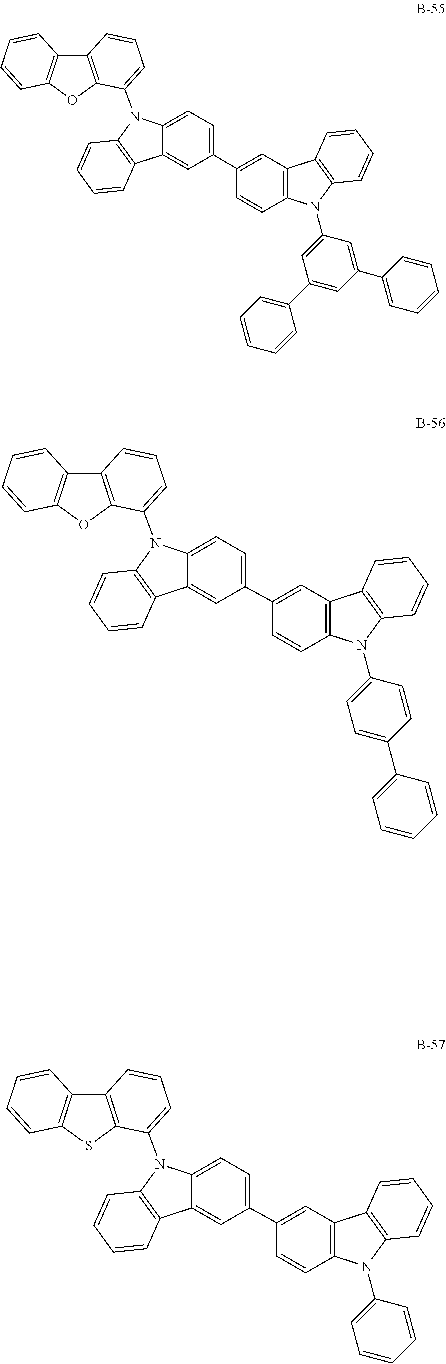 [SMILES: c1ccc(-c2cc(-c3ccccc3)cc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5cccc6c5oc5ccccc56)ccc43)cc2)cc1 ; c1ccc(-n2c3ccccc3c3cc(-c4ccc5c(c4)c4ccccc4n5-c4cccc5c4sc4ccccc45)ccc32)cc1]

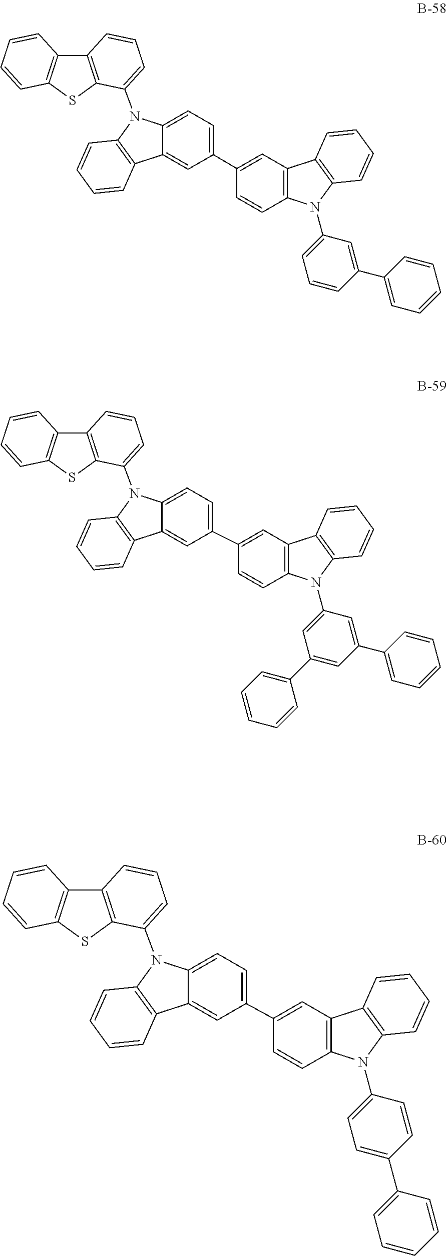 [SMILES: c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1 ; c1ccc(-c2ccc(-n3c4ccccc4c4cc(-c5ccc6c(c5)c5ccccc5n6-c5cccc6c5sc5ccccc56)ccc43)cc2)cc1]

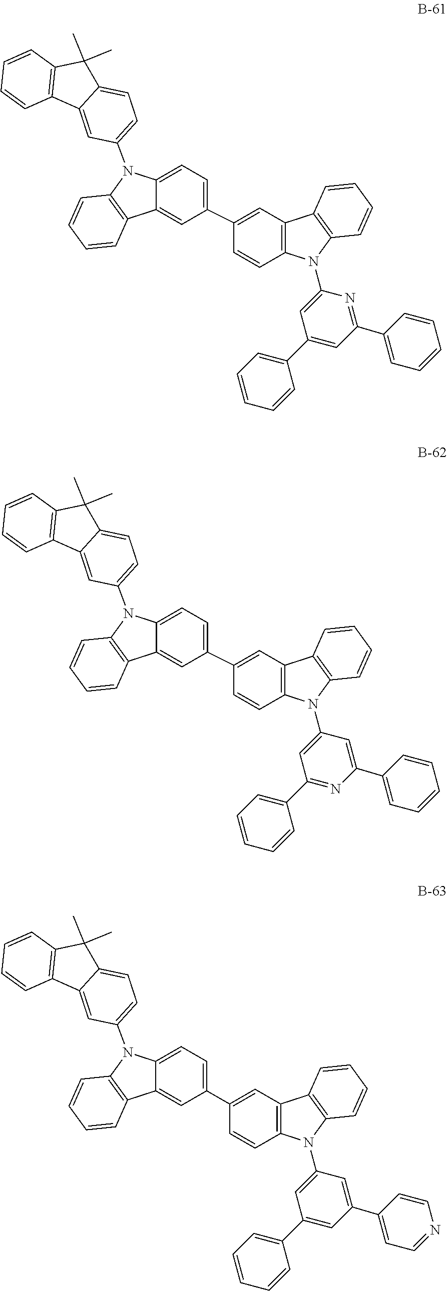 [SMILES: CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21 ; CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccncc6)c5)ccc43)ccc21]

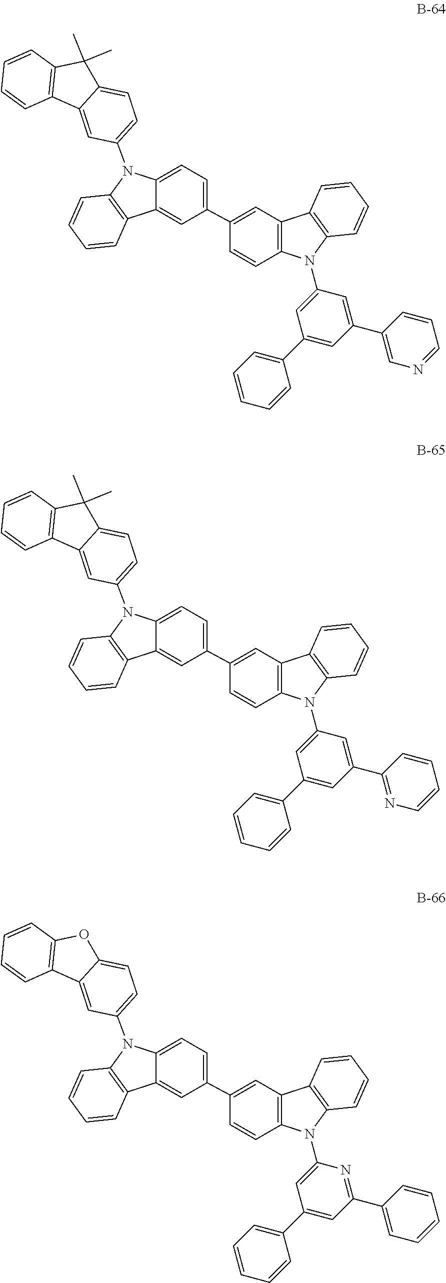 [SMILES: CC1(C)c2ccccc2-c2cc(-n3c4ccccc4c4cc(-c5ccc6c(c5)c5ccccc5n6-c5cc(-c6ccccc6)cc(-c6cccnc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccn6)c5)ccc43)ccc21 ; c1ccc(-c2cc(-c3ccccc3)nc(-n3c4ccccc4c4cc(-c5ccc6c(c5)c5ccccc5n6-c5ccc6oc7ccccc7c6c5)ccc43)c2)cc1]

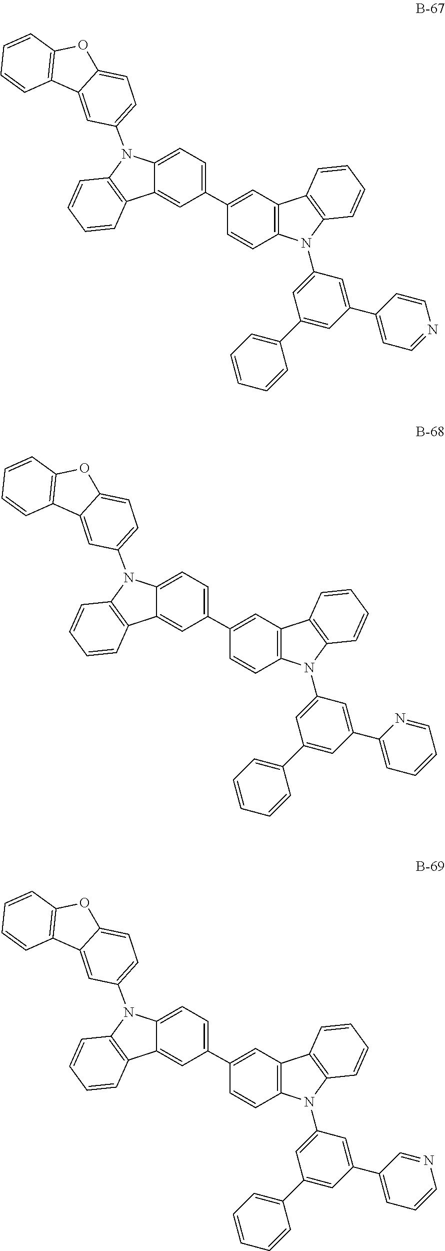 [SMILES: c1ccc(-c2cc(-c3ccncc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1]

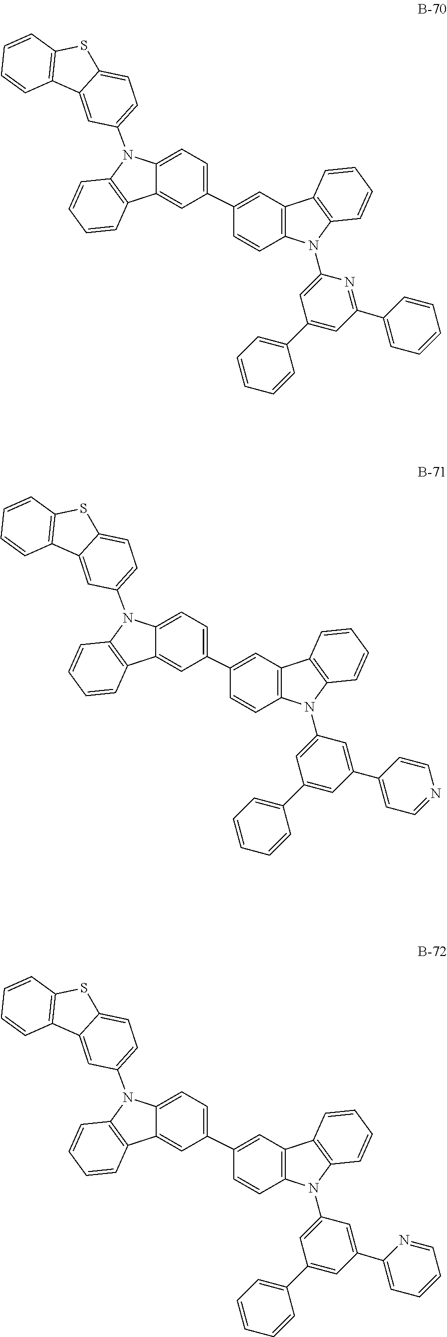 [SMILES: c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1]

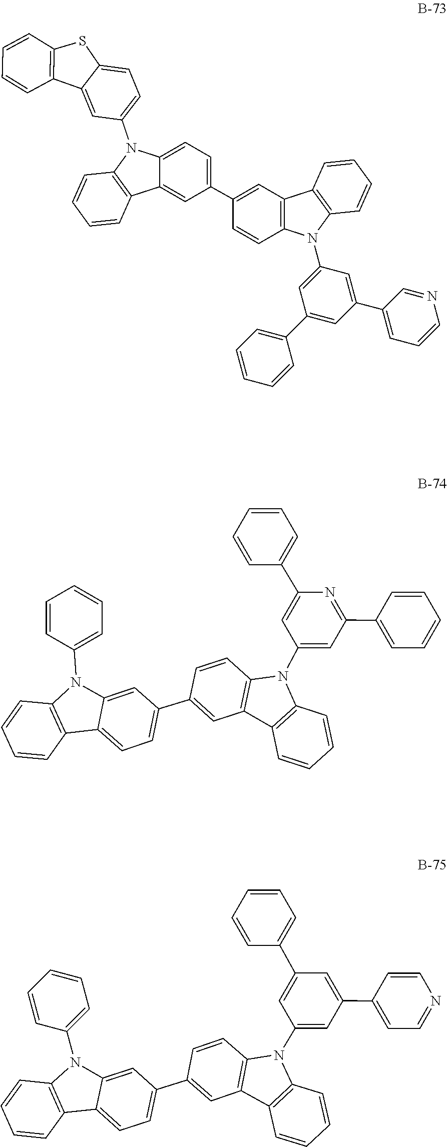 [SMILES: c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1 ; c1ccc(-c2cc(-c3ccncc3)cc(-n3c4ccccc4c4cc(-c5ccc6c7ccccc7n(-c7ccccc7)c6c5)ccc43)c2)cc1]

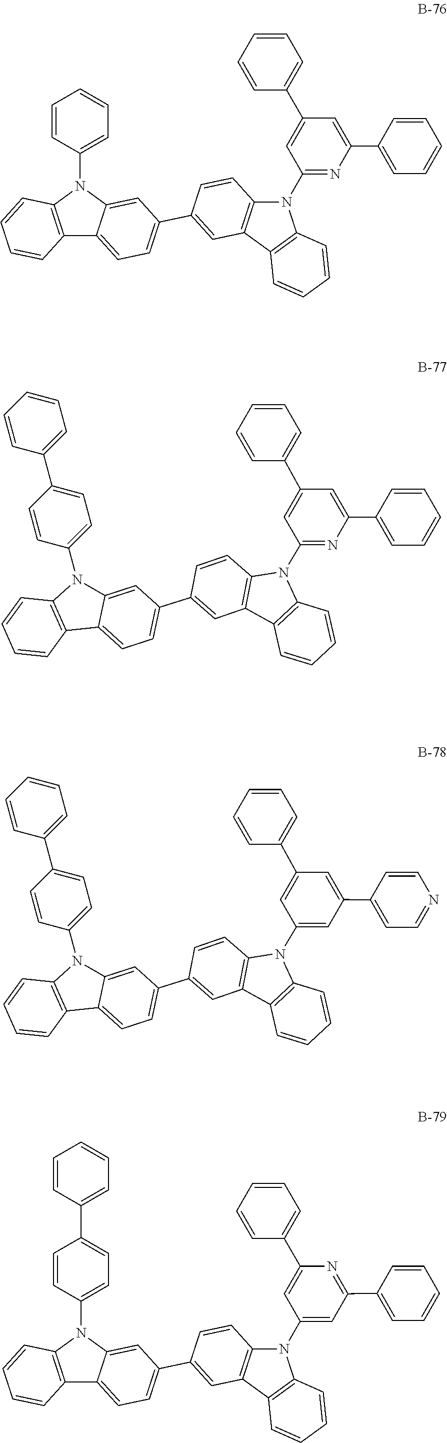 [SMILES: c1ccc(-c2cc(-c3ccccc3)nc(-n3c4ccccc4c4cc(-c5ccc6c7ccccc7n(-c7ccccc7)c6c5)ccc43)c2)cc1 ; c1ccc(-c2ccc(-n3c4ccccc4c4ccc(-c5ccc6c(c5)c5ccccc5n6-c5cc(-c6ccccc6)cc(-c6ccccc6)n5)cc43)cc2)cc1 ; c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1 ; c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1]

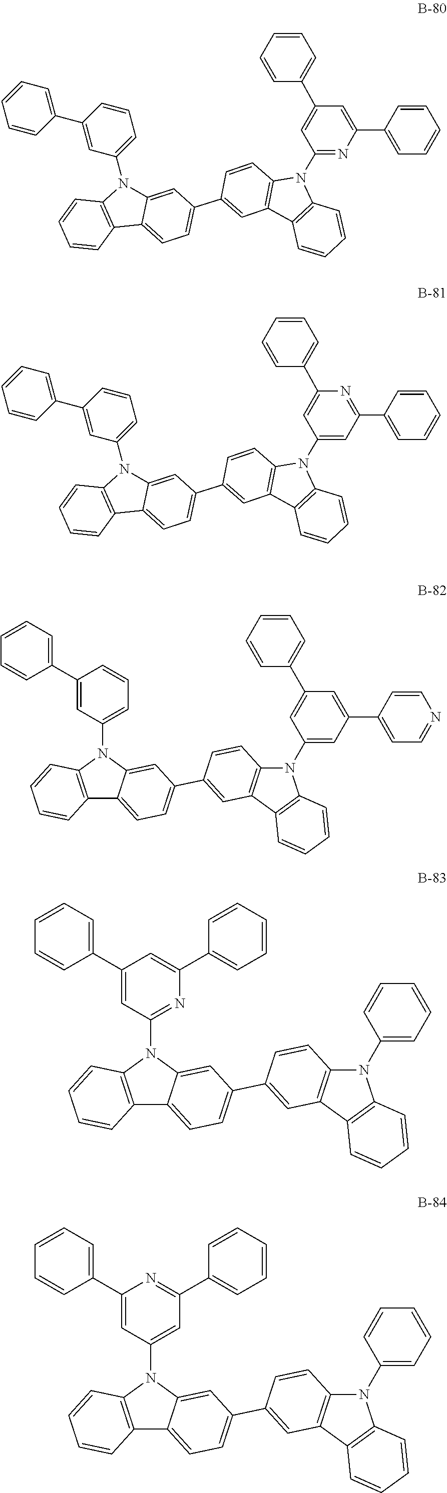 [SMILES: c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1 ; c1ccc(-c2cccc(-n3c4ccccc4c4ccc(-c5ccc6c(c5)c5ccccc5n6-c5cc(-c6ccccc6)nc(-c6ccccc6)c5)cc43)c2)cc1 ; c1ccc(-c2cccc(-n3c4ccccc4c4ccc(-c5ccc6c(c5)c5ccccc5n6-c5cc(-c6ccccc6)cc(-c6ccncc6)c5)cc43)c2)cc1 ; c1ccc(-c2cc(-c3ccccc3)nc(-n3c4ccccc4c4ccc(-c5ccc6c(c5)c5ccccc5n6-c5ccccc5)cc43)c2)cc1 ; c1ccc(-c2cc(-n3c4ccccc4c4ccc(-c5ccc6c(c5)c5ccccc5n6-c5ccccc5)cc43)cc(-c3ccccc3)n2)cc1]

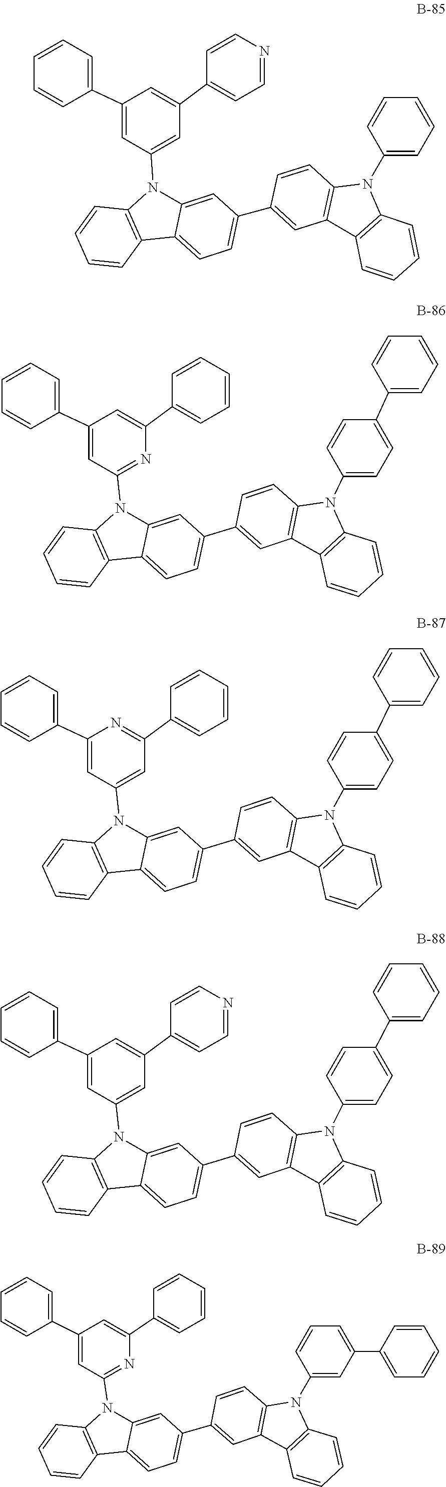 [SMILES: c1ccc(-c2cc(-c3ccncc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1 ; c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1 ; c1ccc(-c2ccc(-n3c4ccccc4c4cc(-c5ccc6c7ccccc7n(-c7cc(-c8ccccc8)cc(-c8ccncc8)c7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1]

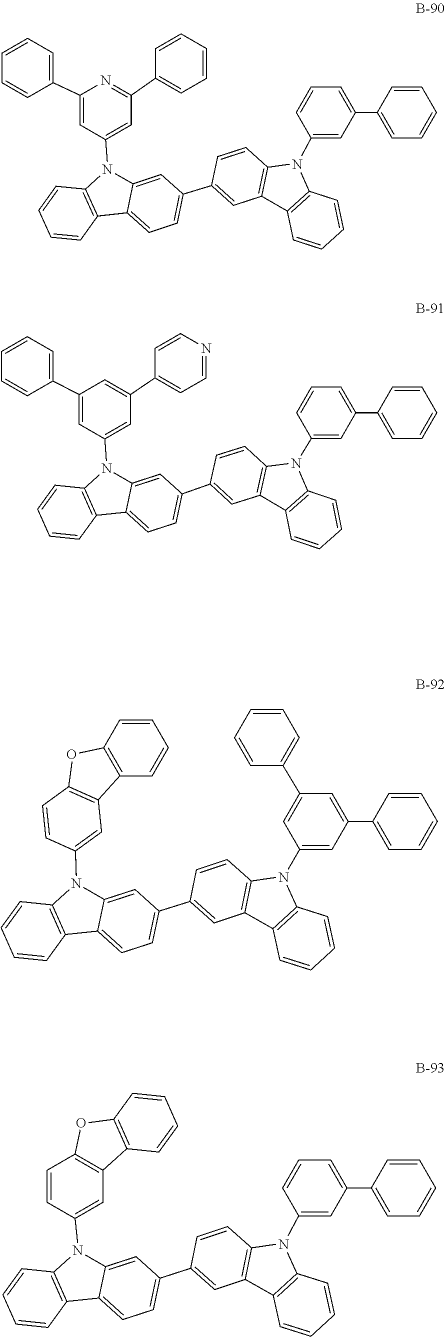 [SMILES: c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1 ; c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1 ; c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1 ; c1ccc(-c2cccc(-n3c4ccccc4c4cc(-c5ccc6c7ccccc7n(-c7ccc8oc9ccccc9c8c7)c6c5)ccc43)c2)cc1]

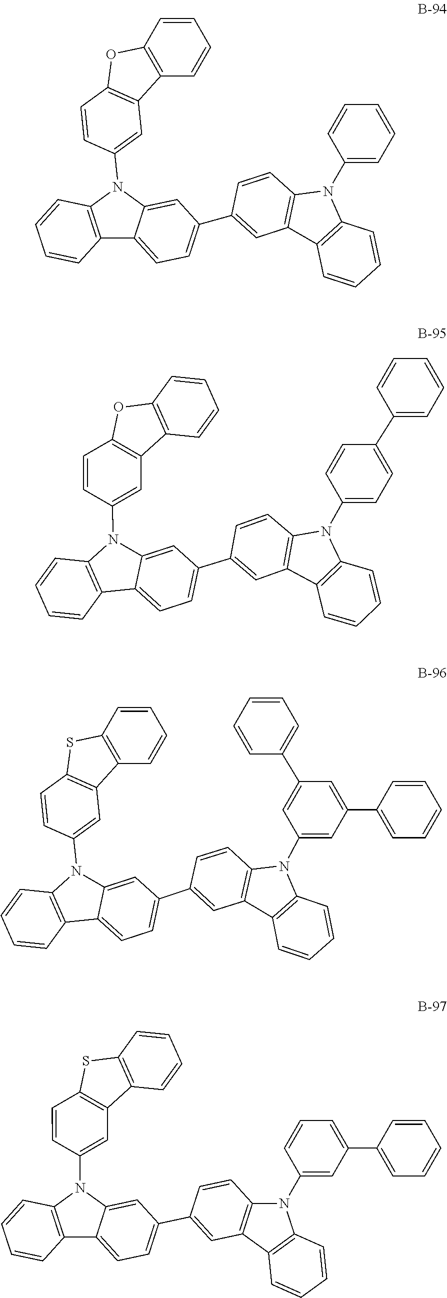 [SMILES: c1ccc(-n2c3ccccc3c3cc(-c4ccc5c6ccccc6n(-c6ccc7oc8ccccc8c7c6)c5c4)ccc32)cc1 ; c1ccc(-c2ccc(-n3c4ccccc4c4cc(-c5ccc6c7ccccc7n(-c7ccc8oc9ccccc9c8c7)c6c5)ccc43)cc2)cc1 ; c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1 ; c1ccc(-c2cccc(-n3c4ccccc4c4cc(-c5ccc6c7ccccc7n(-c7ccc8sc9ccccc9c8c7)c6c5)ccc43)c2)cc1]

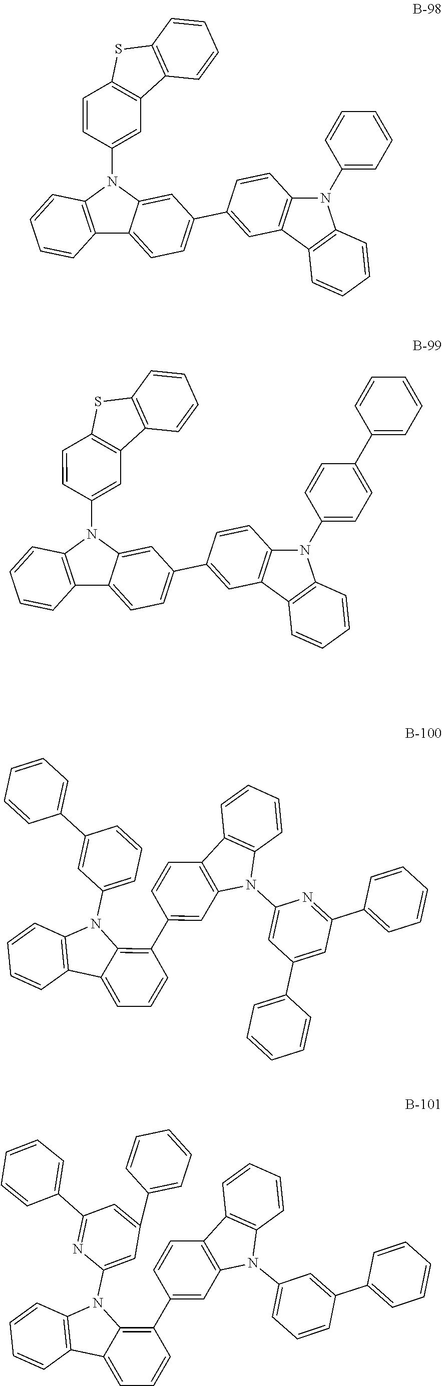 [SMILES: c1ccc(-n2c3ccccc3c3cc(-c4ccc5c6ccccc6n(-c6ccc7sc8ccccc8c7c6)c5c4)ccc32)cc1 ; c1ccc(-c2ccc(-n3c4ccccc4c4cc(-c5ccc6c7ccccc7n(-c7ccc8sc9ccccc9c8c7)c6c5)ccc43)cc2)cc1 ; c1ccc(-c2cccc(-n3c4ccccc4c4cccc(-c5ccc6c7ccccc7n(-c7cc(-c8ccccc8)cc(-c8ccccc8)n7)c6c5)c43)c2)cc1 ; c1ccc(-c2cccc(-n3c4ccccc4c4ccc(-c5cccc6c7ccccc7n(-c7cc(-c8ccccc8)cc(-c8ccccc8)n7)c56)cc43)c2)cc1]

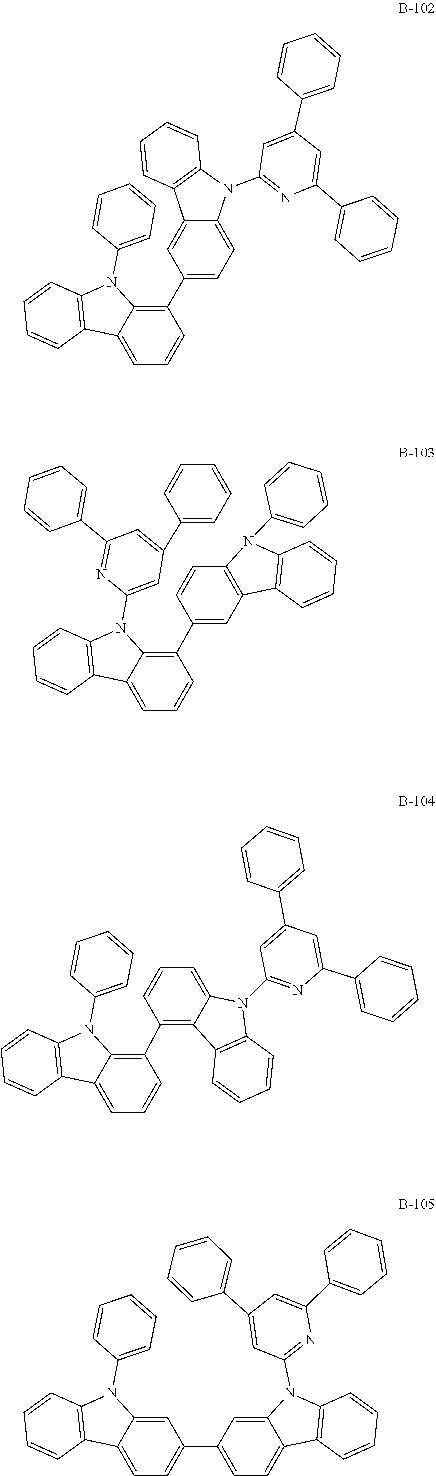 [SMILES: c1ccc(-c2cc(-c3ccccc3)nc(-n3c4ccccc4c4cc(-c5cccc6c7ccccc7n(-c7ccccc7)c56)ccc43)c2)cc1 ; c1ccc(-c2cc(-c3ccccc3)nc(-n3c4ccccc4c4cccc(-c5ccc6c(c5)c5ccccc5n6-c5ccccc5)c43)c2)cc1 ; c1ccc(-c2cc(-c3ccccc3)nc(-n3c4ccccc4c4c(-c5cccc6c7ccccc7n(-c7ccccc7)c56)cccc43)c2)cc1 ; c1ccc(-c2cc(-c3ccccc3)nc(-n3c4ccccc4c4ccc(-c5ccc6c7ccccc7n(-c7ccccc7)c6c5)cc43)c2)cc1]

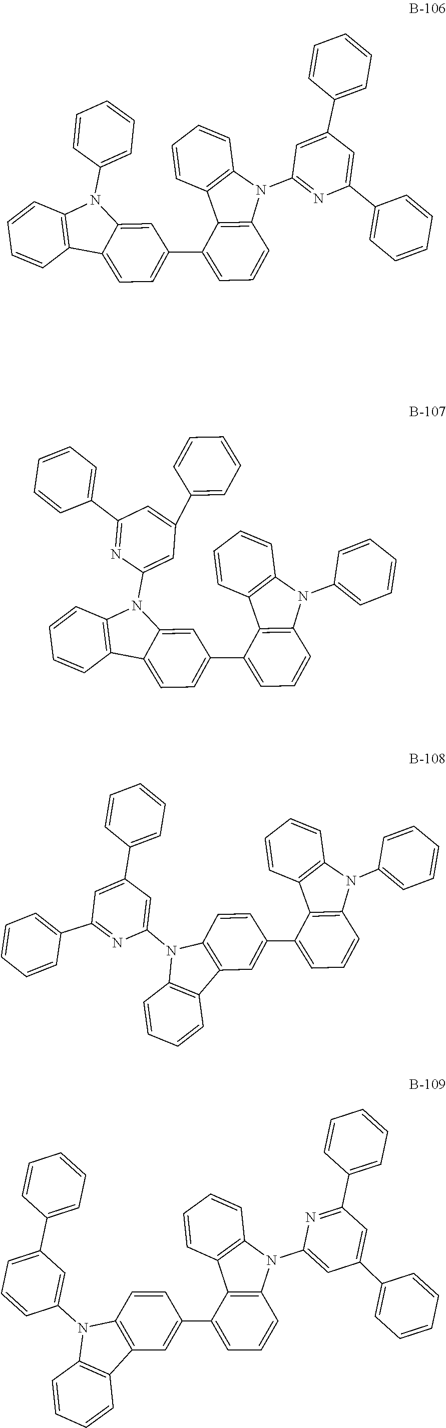 [SMILES: c1ccc(-c2cc(-c3ccccc3)nc(-n3c4ccccc4c4c(-c5ccc6c7ccccc7n(-c7ccccc7)c6c5)cccc43)c2)cc1 ; c1ccc(-c2cc(-c3ccccc3)nc(-n3c4ccccc4c4ccc(-c5cccc6c5c5ccccc5n6-c5ccccc5)cc43)c2)cc1 ; c1ccc(-c2cc(-c3ccccc3)nc(-n3c4ccccc4c4cc(-c5cccc6c5c5ccccc5n6-c5ccccc5)ccc43)c2)cc1 ; c1ccc(-c2cccc(-n3c4ccccc4c4cc(-c5cccc6c5c5ccccc5n6-c5cc(-c6ccccc6)cc(-c6ccccc6)n5)ccc43)c2)cc1]

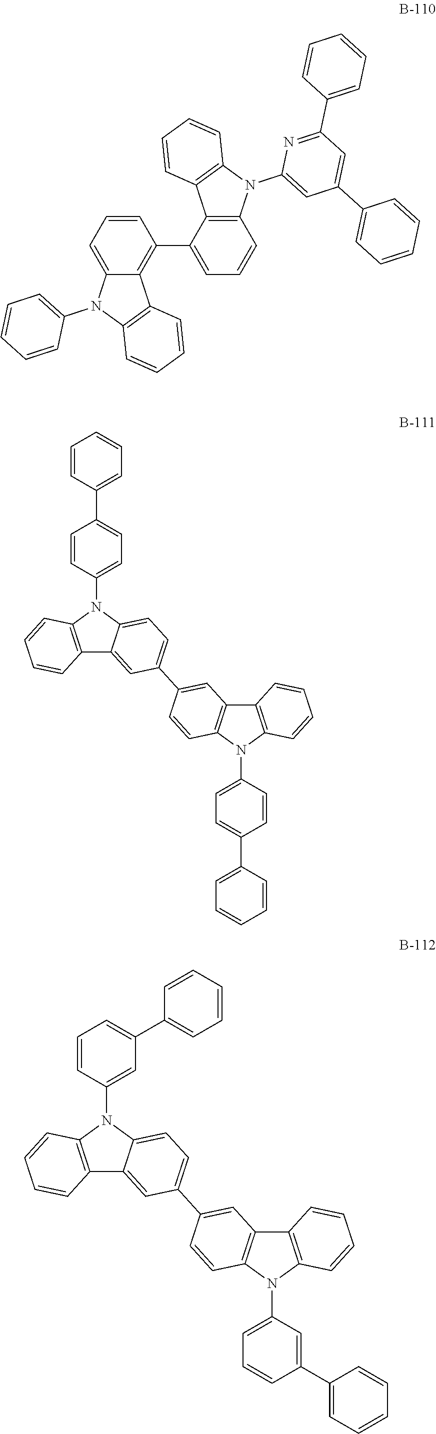 [SMILES: c1ccc(-c2cc(-c3ccccc3)nc(-n3c4ccccc4c4c(-c5cccc6c5c5ccccc5n6-c5ccccc5)cccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

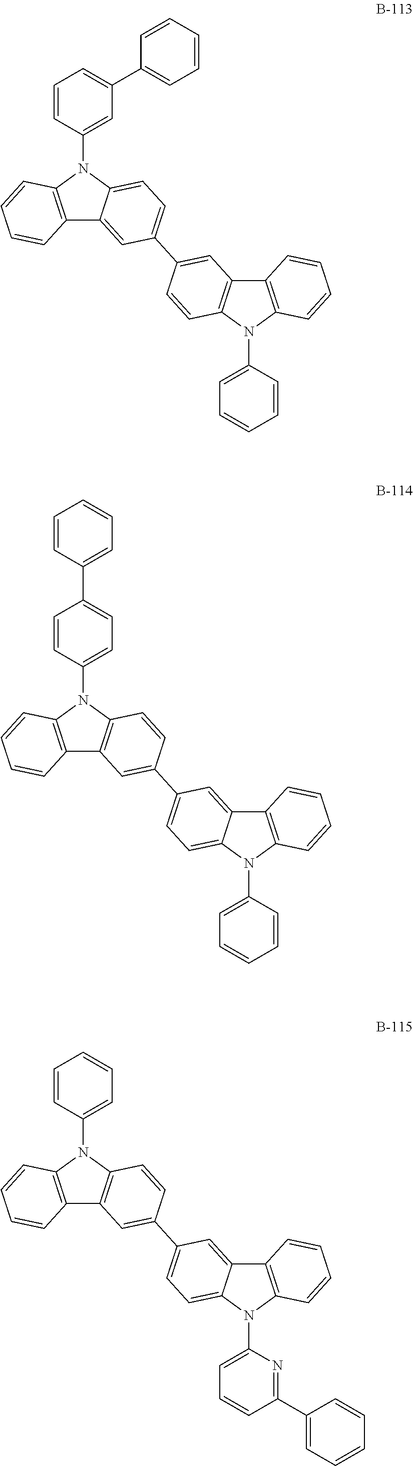 [SMILES: c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)n2)cc1]

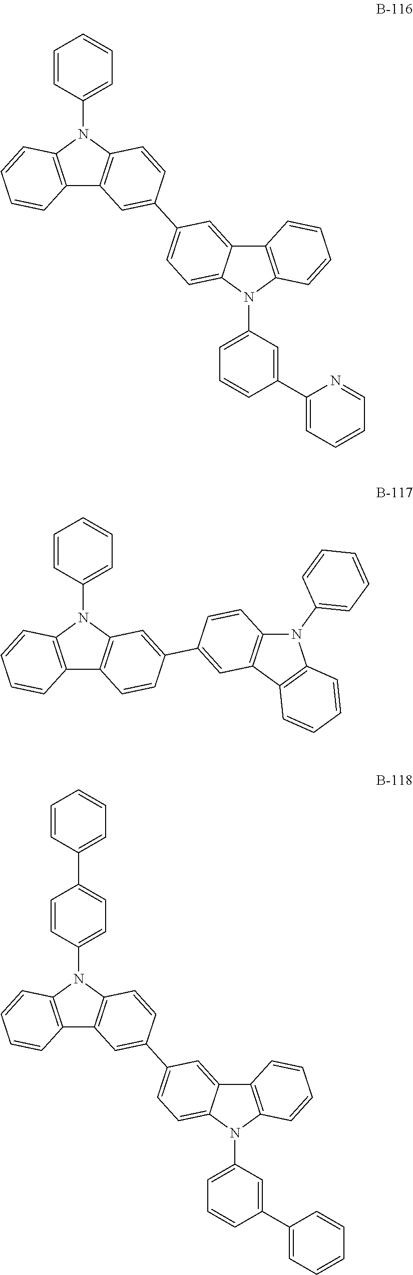 [SMILES: c1ccc(-n2c3ccccc3c3cc(-c4ccc5c(c4)c4ccccc4n5-c4cccc(-c5ccccn5)c4)ccc32)cc1 ; c1ccc(-n2c3ccccc3c3cc(-c4ccc5c6ccccc6n(-c6ccccc6)c5c4)ccc32)cc1 ; c1ccc(-c2ccc(-n3c4ccccc4c4cc(-c5ccc6c(c5)c5ccccc5n6-c5cccc(-c6ccccc6)c5)ccc43)cc2)cc1]

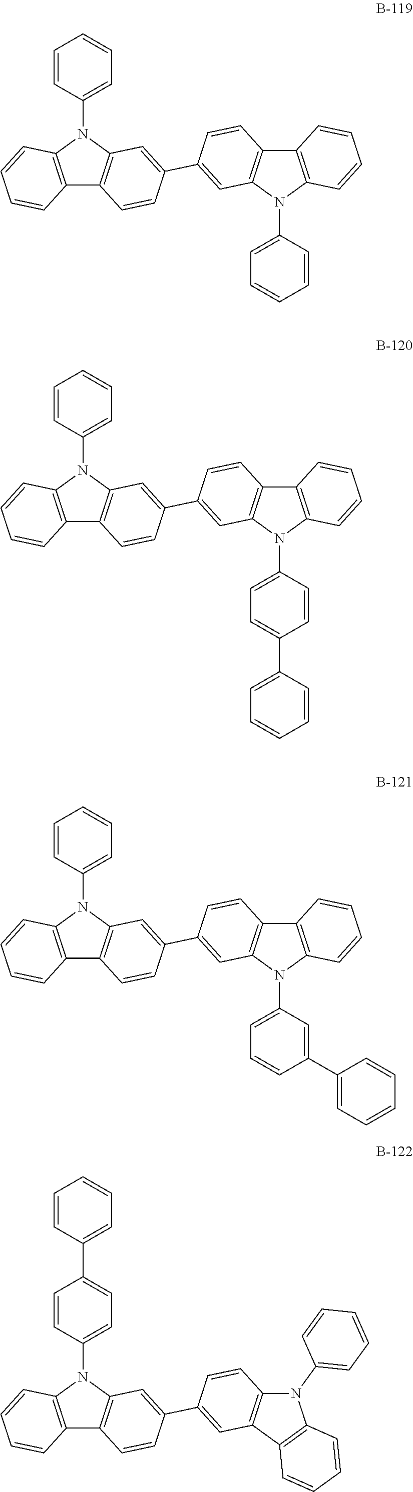 [SMILES: c1ccc(-n2c3ccccc3c3ccc(-c4ccc5c6ccccc6n(-c6ccccc6)c5c4)cc32)cc1 ; c1ccc(-c2ccc(-n3c4ccccc4c4ccc(-c5ccc6c7ccccc7n(-c7ccccc7)c6c5)cc43)cc2)cc1 ; c1ccc(-c2cccc(-n3c4ccccc4c4ccc(-c5ccc6c7ccccc7n(-c7ccccc7)c6c5)cc43)c2)cc1 ; c1ccc(-c2ccc(-n3c4ccccc4c4ccc(-c5ccc6c(c5)c5ccccc5n6-c5ccccc5)cc43)cc2)cc1]

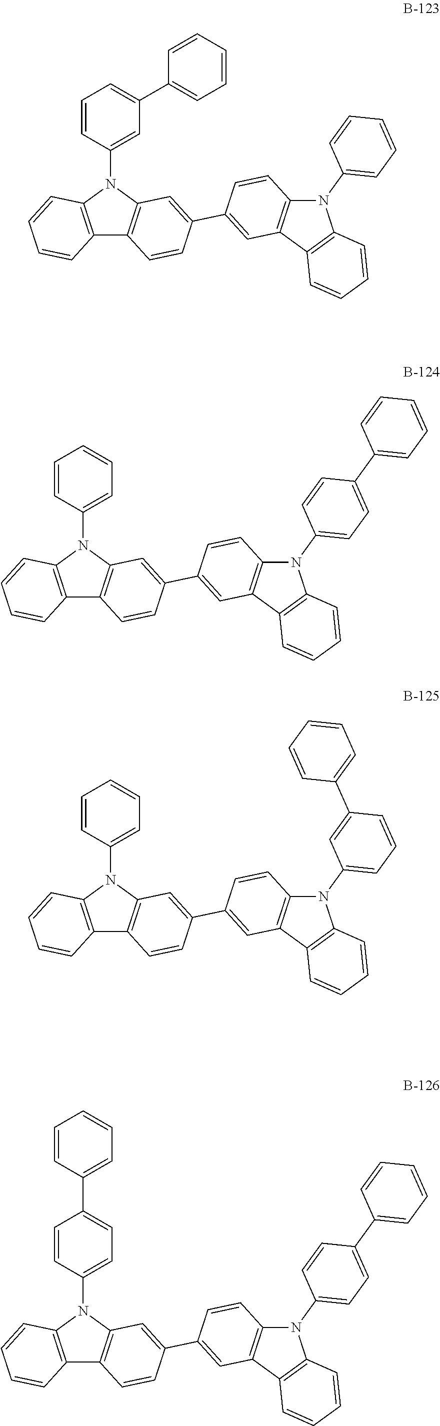 [SMILES: c1ccc(-c2cccc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7ccccc7)c6c5)ccc43)cc2)cc1 ; c1ccc(-c2cccc(-n3c4ccccc4c4cc(-c5ccc6c7ccccc7n(-c7ccccc7)c6c5)ccc43)c2)cc1 ; c1ccc(-c2ccc(-n3c4ccccc4c4cc(-c5ccc6c7ccccc7n(-c7ccc(-c8ccccc8)cc7)c6c5)ccc43)cc2)cc1]

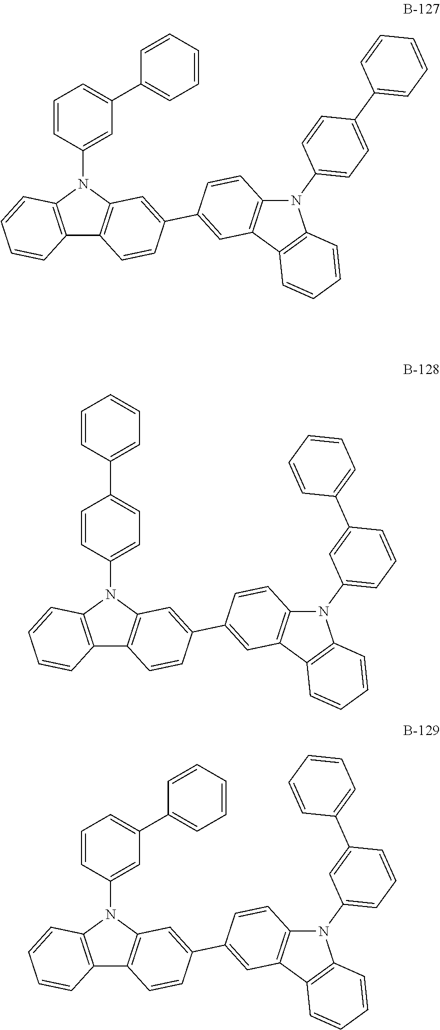 [SMILES: c1ccc(-c2ccc(-n3c4ccccc4c4cc(-c5ccc6c7ccccc7n(-c7cccc(-c8ccccc8)c7)c6c5)ccc43)cc2)cc1 ; c1ccc(-c2ccc(-n3c4ccccc4c4ccc(-c5ccc6c(c5)c5ccccc5n6-c5cccc(-c6ccccc6)c5)cc43)cc2)cc1 ; c1ccc(-c2cccc(-n3c4ccccc4c4cc(-c5ccc6c7ccccc7n(-c7cccc(-c8ccccc8)c7)c6c5)ccc43)c2)cc1]

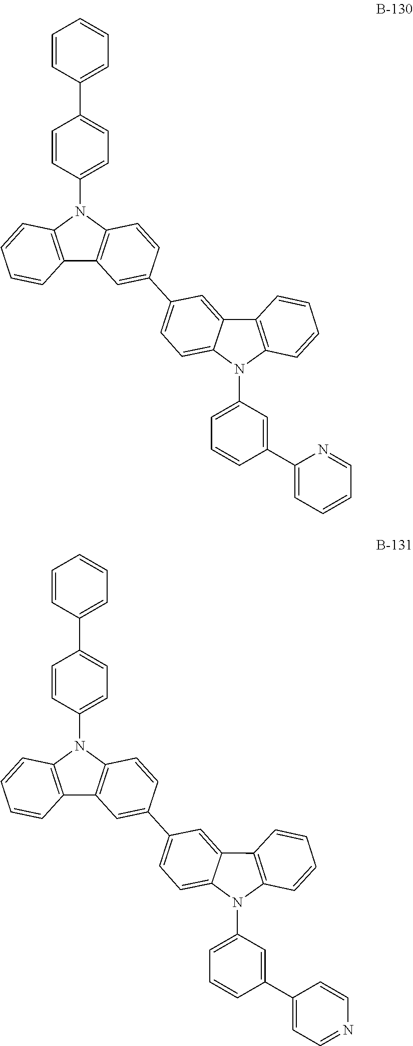 [SMILES: c1ccc(-c2ccc(-n3c4ccccc4c4cc(-c5ccc6c(c5)c5ccccc5n6-c5cccc(-c6ccccn6)c5)ccc43)cc2)cc1 ; c1ccc(-c2ccc(-n3c4ccccc4c4cc(-c5ccc6c(c5)c5ccccc5n6-c5cccc(-c6ccncc6)c5)ccc43)cc2)cc1]

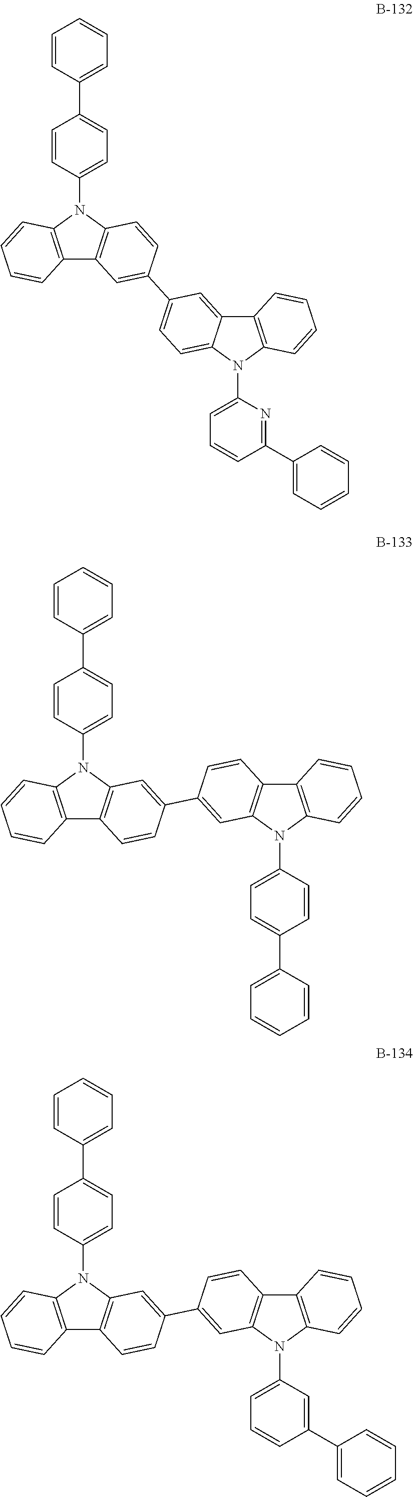 [SMILES: c1ccc(-c2ccc(-n3c4ccccc4c4cc(-c5ccc6c(c5)c5ccccc5n6-c5cccc(-c6ccccc6)n5)ccc43)cc2)cc1 ; c1ccc(-c2ccc(-n3c4ccccc4c4ccc(-c5ccc6c7ccccc7n(-c7ccc(-c8ccccc8)cc7)c6c5)cc43)cc2)cc1 ; c1ccc(-c2ccc(-n3c4ccccc4c4ccc(-c5ccc6c7ccccc7n(-c7cccc(-c8ccccc8)c7)c6c5)cc43)cc2)cc1]

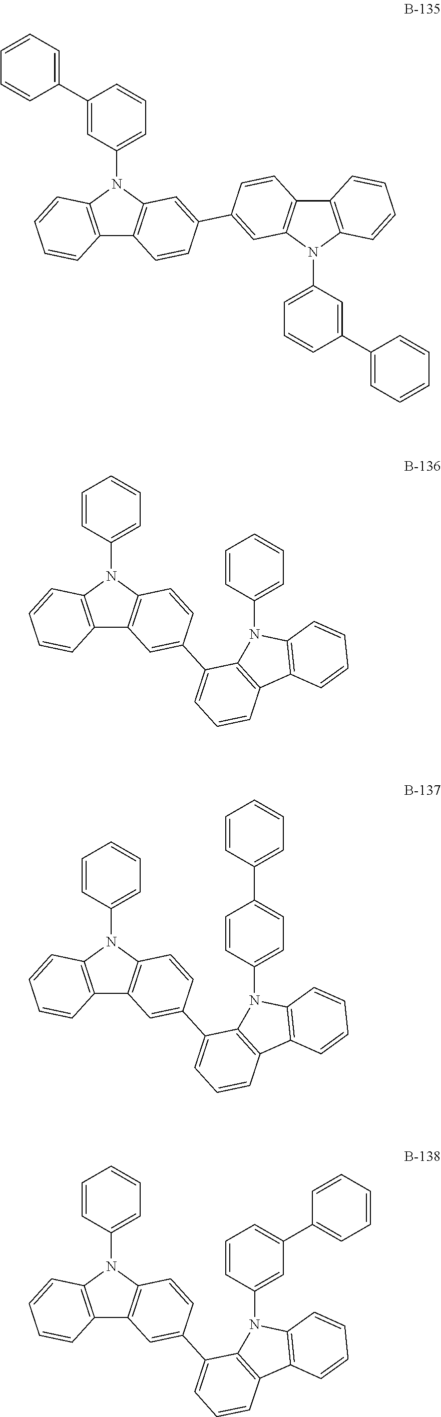 [SMILES: c1ccc(-c2cccc(-n3c4ccccc4c4ccc(-c5ccc6c7ccccc7n(-c7cccc(-c8ccccc8)c7)c6c5)cc43)c2)cc1 ; c1ccc(-n2c3ccccc3c3cc(-c4cccc5c6ccccc6n(-c6ccccc6)c45)ccc32)cc1 ; c1ccc(-c2ccc(-n3c4ccccc4c4cccc(-c5ccc6c(c5)c5ccccc5n6-c5ccccc5)c43)cc2)cc1 ; c1ccc(-c2cccc(-n3c4ccccc4c4cccc(-c5ccc6c(c5)c5ccccc5n6-c5ccccc5)c43)c2)cc1]

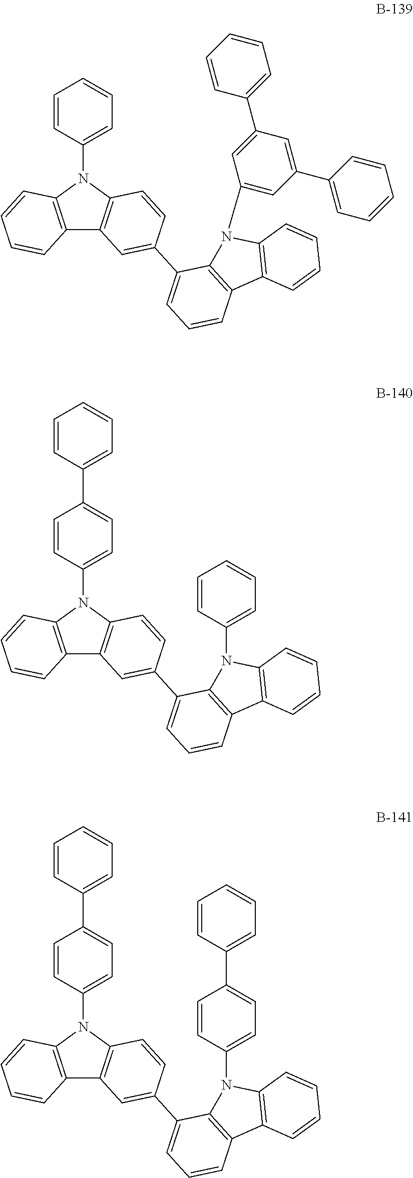 [SMILES: c1ccc(-c2cc(-c3ccccc3)cc(-n3c4ccccc4c4cccc(-c5ccc6c(c5)c5ccccc5n6-c5ccccc5)c43)c2)cc1 ; c1ccc(-c2ccc(-n3c4ccccc4c4cc(-c5cccc6c7ccccc7n(-c7ccccc7)c56)ccc43)cc2)cc1 ; c1ccc(-c2ccc(-n3c4ccccc4c4cc(-c5cccc6c7ccccc7n(-c7ccc(-c8ccccc8)cc7)c56)ccc43)cc2)cc1]

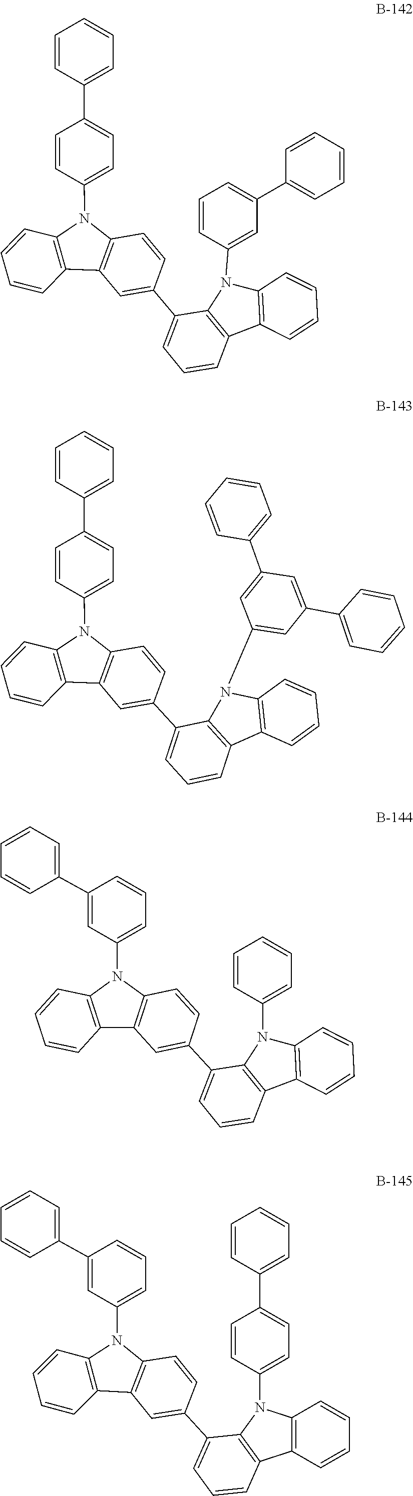 [SMILES: c1ccc(-c2ccc(-n3c4ccccc4c4cc(-c5cccc6c7ccccc7n(-c7cccc(-c8ccccc8)c7)c56)ccc43)cc2)cc1 ; c1ccc(-c2ccc(-n3c4ccccc4c4cc(-c5cccc6c7ccccc7n(-c7cc(-c8ccccc8)cc(-c8ccccc8)c7)c56)ccc43)cc2)cc1 ; c1ccc(-c2cccc(-n3c4ccccc4c4cc(-c5cccc6c7ccccc7n(-c7ccccc7)c56)ccc43)c2)cc1 ; c1ccc(-c2ccc(-n3c4ccccc4c4cccc(-c5ccc6c(c5)c5ccccc5n6-c5cccc(-c6ccccc6)c5)c43)cc2)cc1]

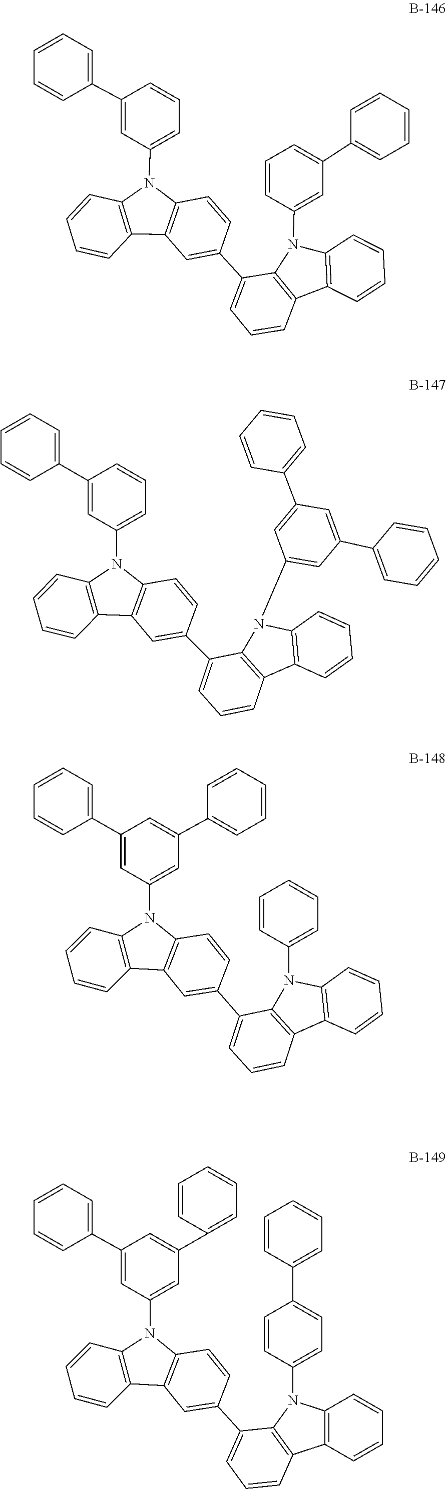 [SMILES: c1ccc(-c2cccc(-n3c4ccccc4c4cc(-c5cccc6c7ccccc7n(-c7cccc(-c8ccccc8)c7)c56)ccc43)c2)cc1 ; c1ccc(-c2cccc(-n3c4ccccc4c4cc(-c5cccc6c7ccccc7n(-c7cc(-c8ccccc8)cc(-c8ccccc8)c7)c56)ccc43)c2)cc1 ; c1ccc(-c2cc(-c3ccccc3)cc(-n3c4ccccc4c4cc(-c5cccc6c7ccccc7n(-c7ccccc7)c56)ccc43)c2)cc1 ; c1ccc(-c2ccc(-n3c4ccccc4c4cccc(-c5ccc6c(c5)c5ccccc5n6-c5cc(-c6ccccc6)cc(-c6ccccc6)c5)c43)cc2)cc1]

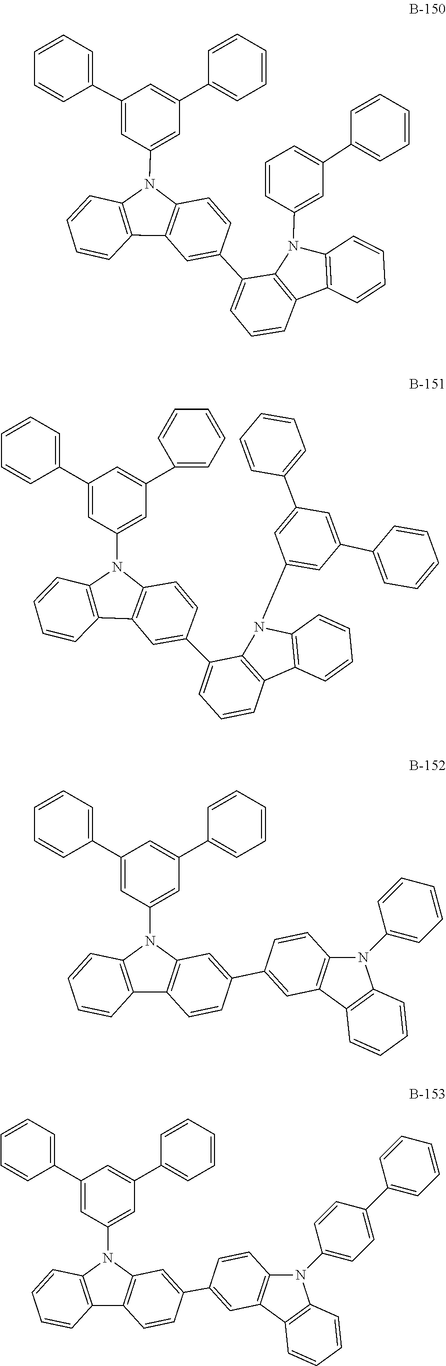 [SMILES: c1ccc(-c2cc(-c3ccccc3)cc(-n3c4ccccc4c4cc(-c5cccc6c7ccccc7n(-c7cccc(-c8ccccc8)c7)c56)ccc43)c2)cc1 ; c1ccc(-c2cc(-c3ccccc3)cc(-n3c4ccccc4c4cc(-c5cccc6c7ccccc7n(-c7cc(-c8ccccc8)cc(-c8ccccc8)c7)c56)ccc43)c2)cc1 ; c1ccc(-c2cc(-c3ccccc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)c7)c6c5)ccc43)cc2)cc1]

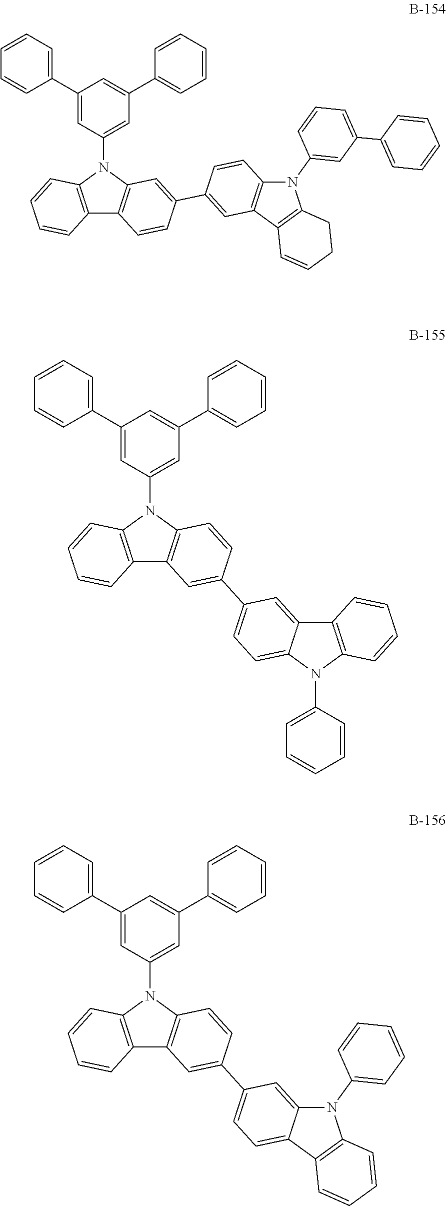 [SMILES: C1=Cc2c(n(-c3cccc(-c4ccccc4)c3)c3ccc(-c4ccc5c6ccccc6n(-c6cc(-c7ccccc7)cc(-c7ccccc7)c6)c5c4)cc23)CC1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cc(-c3ccccc3)cc(-n3c4ccccc4c4cc(-c5ccc6c7ccccc7n(-c7ccccc7)c6c5)ccc43)c2)cc1]

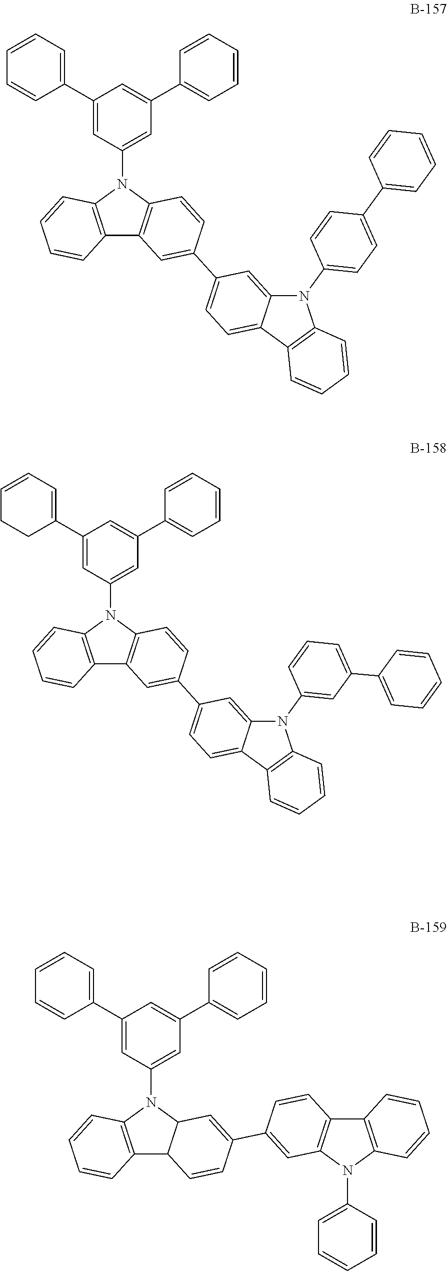 [SMILES: c1ccc(-c2ccc(-n3c4ccccc4c4ccc(-c5ccc6c(c5)c5ccccc5n6-c5cc(-c6ccccc6)cc(-c6ccccc6)c5)cc43)cc2)cc1 ; C1=CCCC(c2cc(-c3ccccc3)cc(-n3c4ccccc4c4cc(-c5ccc6c7ccccc7n(-c7cccc(-c8ccccc8)c7)c6c5)ccc43)c2)=C1 ; C1=CC2c3ccccc3N(c3cc(-c4ccccc4)cc(-c4ccccc4)c3)C2C=C1c1ccc2c3ccccc3n(-c3ccccc3)c2c1]

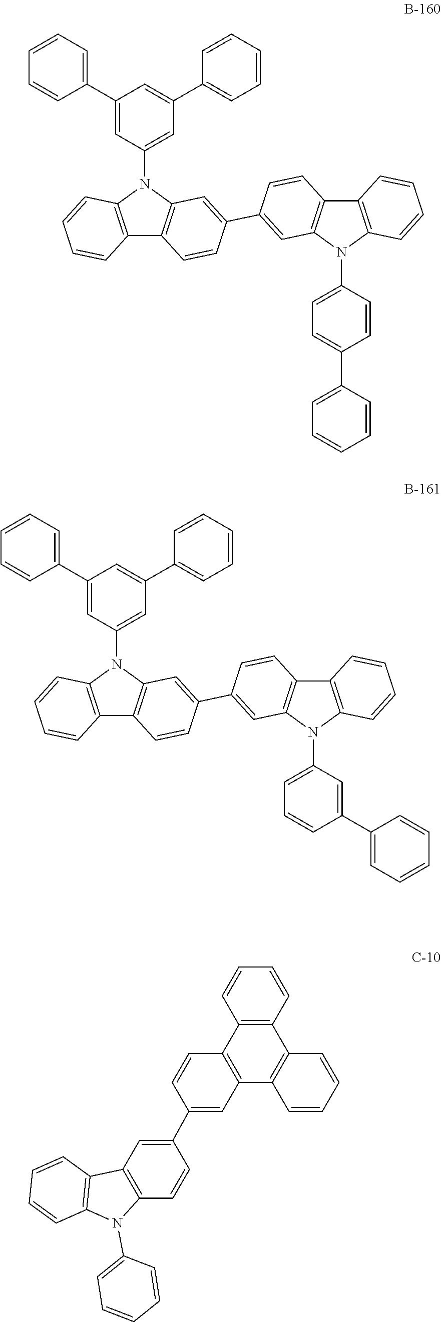 [SMILES: c1ccc(-c2ccc(-n3c4ccccc4c4ccc(-c5ccc6c7ccccc7n(-c7cc(-c8ccccc8)cc(-c8ccccc8)c7)c6c5)cc43)cc2)cc1 ; c1ccc(-c2cccc(-n3c4ccccc4c4ccc(-c5ccc6c7ccccc7n(-c7cc(-c8ccccc8)cc(-c8ccccc8)c7)c6c5)cc43)c2)cc1 ; c1ccc(-n2c3ccccc3c3cc(-c4ccc5c6ccccc6c6ccccc6c5c4)ccc32)cc1]

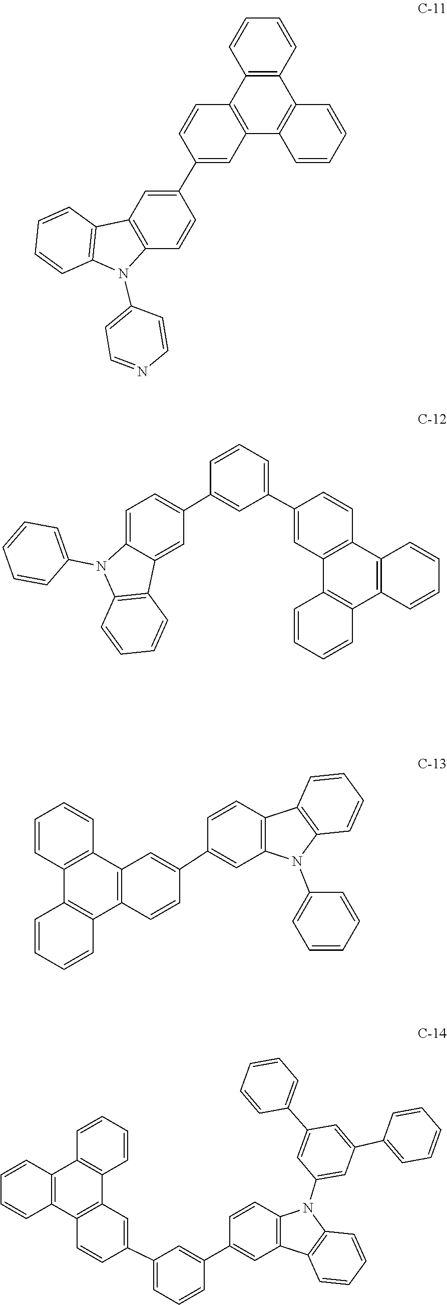 [SMILES: c1ccc2c(c1)c1ccccc1c1cc(-c3ccc4c(c3)c3ccccc3n4-c3ccncc3)ccc21 ; c1ccc(-n2c3ccccc3c3cc(-c4cccc(-c5ccc6c7ccccc7c7ccccc7c6c5)c4)ccc32)cc1 ; c1ccc(-n2c3ccccc3c3ccc(-c4ccc5c6ccccc6c6ccccc6c5c4)cc32)cc1 ; c1ccc(-c2cc(-c3ccccc3)cc(-n3c4ccccc4c4cc(-c5cccc(-c6ccc7c8ccccc8c8ccccc8c7c6)c5)ccc43)c2)cc1]

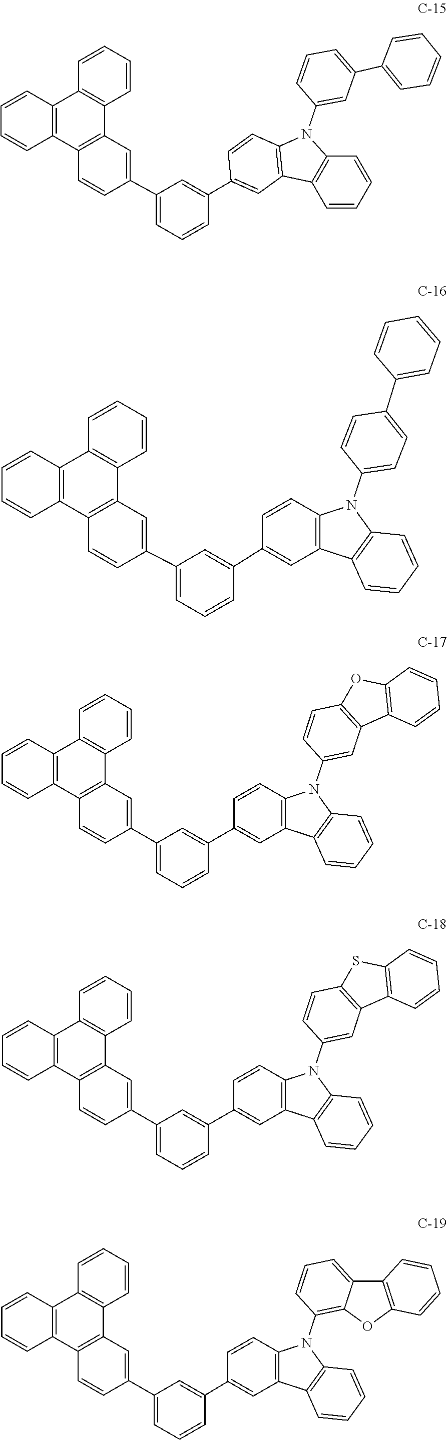 [SMILES: c1ccc(-c2cccc(-n3c4ccccc4c4cc(-c5cccc(-c6ccc7c8ccccc8c8ccccc8c7c6)c5)ccc43)c2)cc1 ; c1ccc(-c2ccc(-n3c4ccccc4c4cc(-c5cccc(-c6ccc7c8ccccc8c8ccccc8c7c6)c5)ccc43)cc2)cc1 ; c1cc(-c2ccc3c4ccccc4c4ccccc4c3c2)cc(-c2ccc3c(c2)c2ccccc2n3-c2ccc3oc4ccccc4c3c2)c1 ; c1cc(-c2ccc3c4ccccc4c4ccccc4c3c2)cc(-c2ccc3c(c2)c2ccccc2n3-c2ccc3sc4ccccc4c3c2)c1 ; c1cc(-c2ccc3c4ccccc4c4ccccc4c3c2)cc(-c2ccc3c(c2)c2ccccc2n3-c2cccc3c2oc2ccccc23)c1]

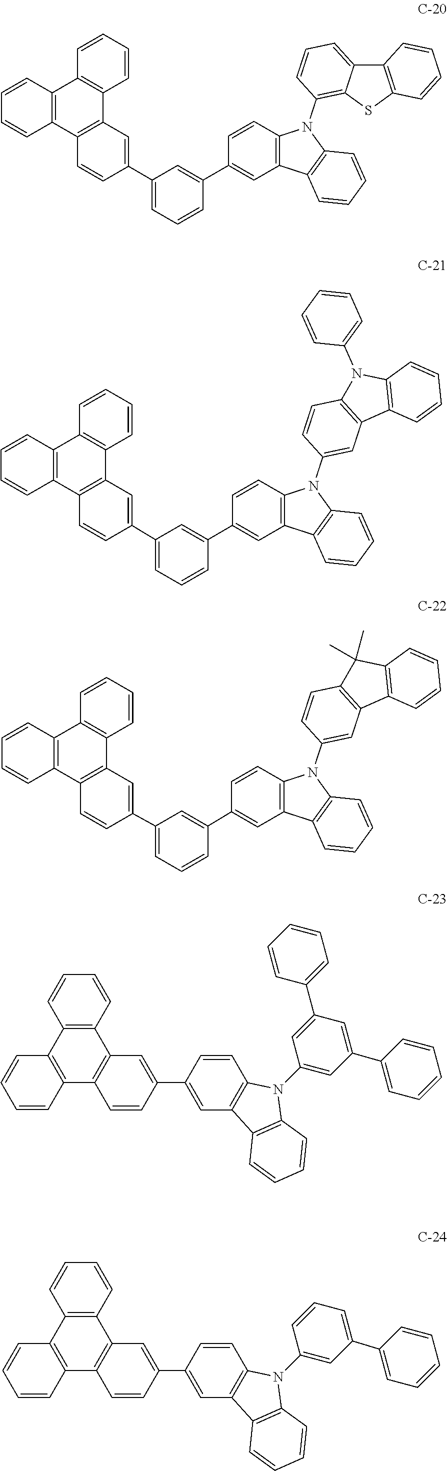 [SMILES: c1cc(-c2ccc3c4ccccc4c4ccccc4c3c2)cc(-c2ccc3c(c2)c2ccccc2n3-c2cccc3c2sc2ccccc23)c1 ; c1ccc(-n2c3ccccc3c3cc(-n4c5ccccc5c5cc(-c6cccc(-c7ccc8c9ccccc9c9ccccc9c8c7)c6)ccc54)ccc32)cc1 ; CC1(C)c2ccccc2-c2cc(-n3c4ccccc4c4cc(-c5cccc(-c6ccc7c8ccccc8c8ccccc8c7c6)c5)ccc43)ccc21 ; c1ccc(-c2cc(-c3ccccc3)cc(-n3c4ccccc4c4cc(-c5ccc6c7ccccc7c7ccccc7c6c5)ccc43)c2)cc1 ; c1ccc(-c2cccc(-n3c4ccccc4c4cc(-c5ccc6c7ccccc7c7ccccc7c6c5)ccc43)c2)cc1]

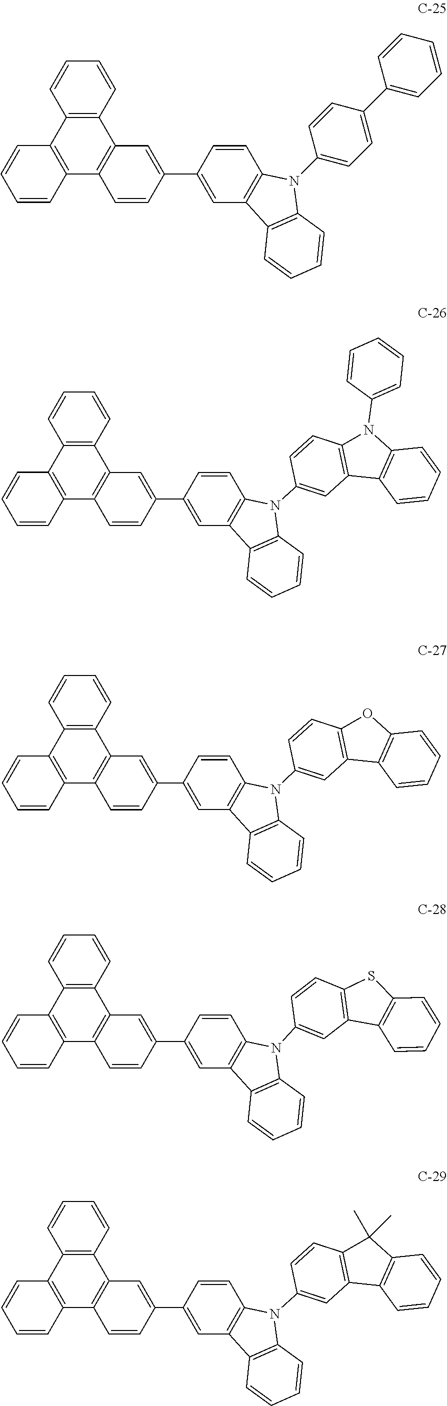 [SMILES: c1ccc(-c2ccc(-n3c4ccccc4c4cc(-c5ccc6c7ccccc7c7ccccc7c6c5)ccc43)cc2)cc1 ; c1ccc(-n2c3ccccc3c3cc(-n4c5ccccc5c5cc(-c6ccc7c8ccccc8c8ccccc8c7c6)ccc54)ccc32)cc1 ; c1ccc2c(c1)oc1ccc(-n3c4ccccc4c4cc(-c5ccc6c7ccccc7c7ccccc7c6c5)ccc43)cc12 ; c1ccc2c(c1)sc1ccc(-n3c4ccccc4c4cc(-c5ccc6c7ccccc7c7ccccc7c6c5)ccc43)cc12 ; CC1(C)c2ccccc2-c2cc(-n3c4ccccc4c4cc(-c5ccc6c7ccccc7c7ccccc7c6c5)ccc43)ccc21]

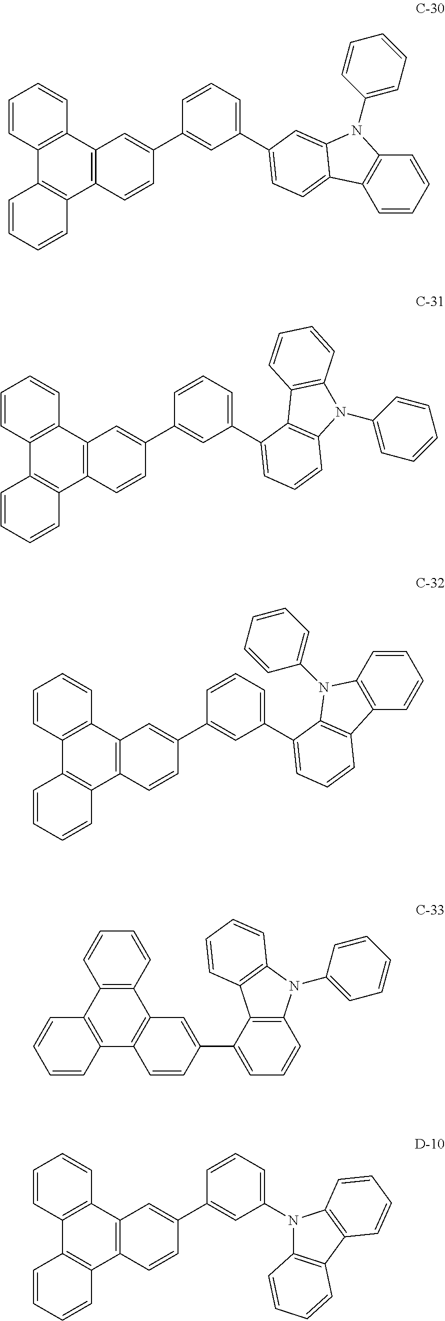 [SMILES: c1ccc(-n2c3ccccc3c3ccc(-c4cccc(-c5ccc6c7ccccc7c7ccccc7c6c5)c4)cc32)cc1 ; c1ccc(-n2c3ccccc3c3c(-c4cccc(-c5ccc6c7ccccc7c7ccccc7c6c5)c4)cccc32)cc1 ; c1ccc(-n2c3ccccc3c3cccc(-c4cccc(-c5ccc6c7ccccc7c7ccccc7c6c5)c4)c32)cc1 ; c1ccc(-n2c3ccccc3c3c(-c4ccc5c6ccccc6c6ccccc6c5c4)cccc32)cc1 ; c1cc(-c2ccc3c4ccccc4c4ccccc4c3c2)cc(-n2c3ccccc3c3ccccc32)c1]

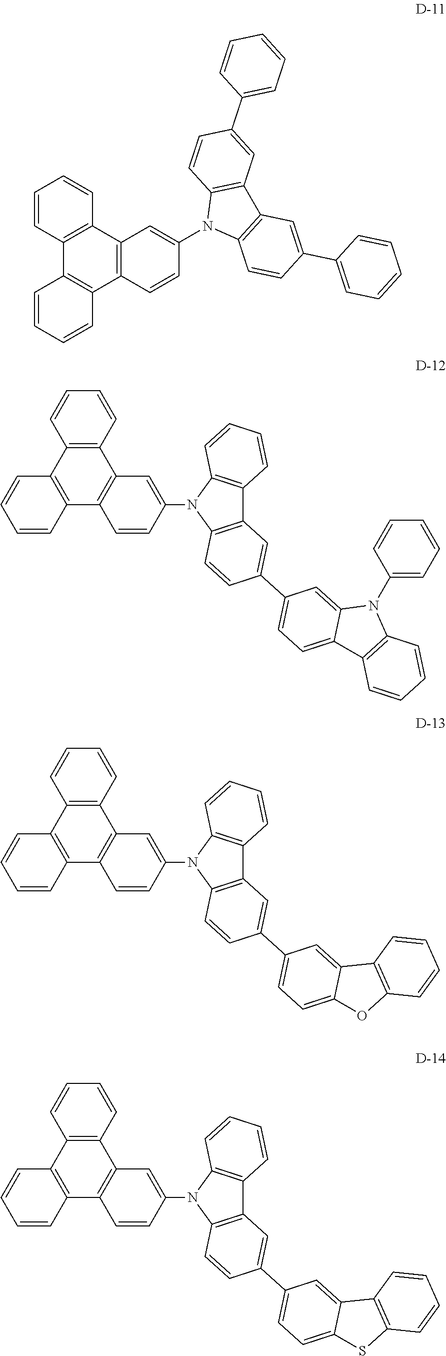 [SMILES: c1ccc(-c2ccc3c(c2)c2cc(-c4ccccc4)ccc2n3-c2ccc3c4ccccc4c4ccccc4c3c2)cc1 ; c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)oc1ccc(-c3ccc4c(c3)c3ccccc3n4-c3ccc4c5ccccc5c5ccccc5c4c3)cc12 ; c1ccc2c(c1)sc1ccc(-c3ccc4c(c3)c3ccccc3n4-c3ccc4c5ccccc5c5ccccc5c4c3)cc12]

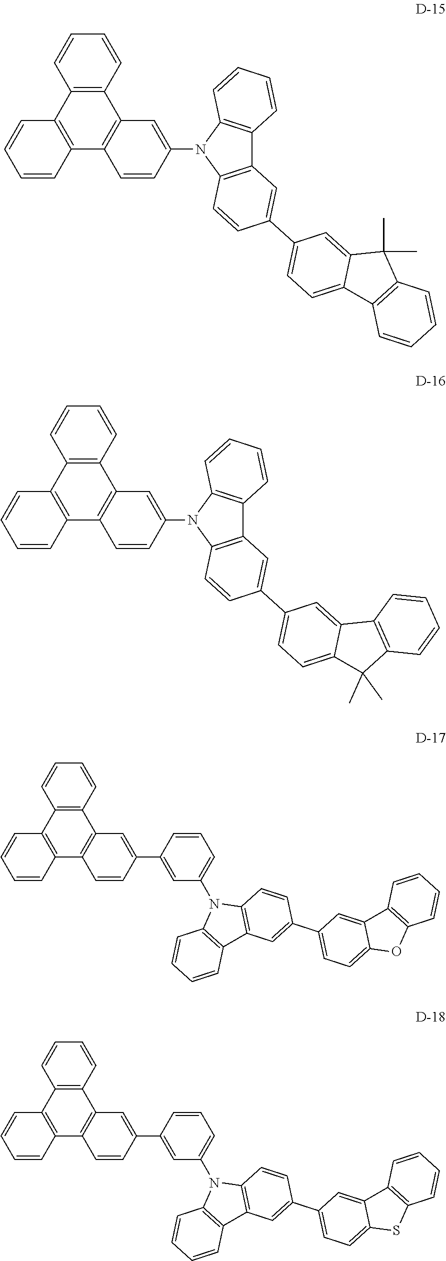 [SMILES: CC1(C)c2ccccc2-c2ccc(-c3ccc4c(c3)c3ccccc3n4-c3ccc4c5ccccc5c5ccccc5c4c3)cc21 ; CC1(C)c2ccccc2-c2cc(-c3ccc4c(c3)c3ccccc3n4-c3ccc4c5ccccc5c5ccccc5c4c3)ccc21 ; c1cc(-c2ccc3c4ccccc4c4ccccc4c3c2)cc(-n2c3ccccc3c3cc(-c4ccc5oc6ccccc6c5c4)ccc32)c1 ; c1cc(-c2ccc3c4ccccc4c4ccccc4c3c2)cc(-n2c3ccccc3c3cc(-c4ccc5sc6ccccc6c5c4)ccc32)c1]

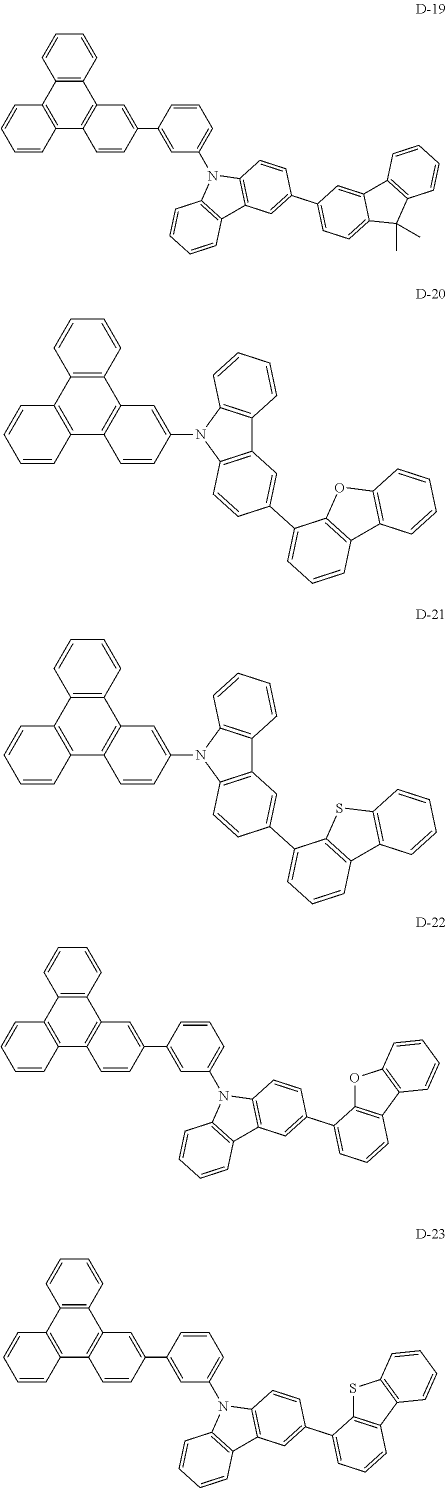 [SMILES: CC1(C)c2ccccc2-c2cc(-c3ccc4c(c3)c3ccccc3n4-c3cccc(-c4ccc5c6ccccc6c6ccccc6c5c4)c3)ccc21 ; c1ccc2c(c1)oc1c(-c3ccc4c(c3)c3ccccc3n4-c3ccc4c5ccccc5c5ccccc5c4c3)cccc12 ; c1ccc2c(c1)sc1c(-c3ccc4c(c3)c3ccccc3n4-c3ccc4c5ccccc5c5ccccc5c4c3)cccc12 ; c1cc(-c2ccc3c4ccccc4c4ccccc4c3c2)cc(-n2c3ccccc3c3cc(-c4cccc5c4oc4ccccc45)ccc32)c1 ; c1cc(-c2ccc3c4ccccc4c4ccccc4c3c2)cc(-n2c3ccccc3c3cc(-c4cccc5c4sc4ccccc45)ccc32)c1]

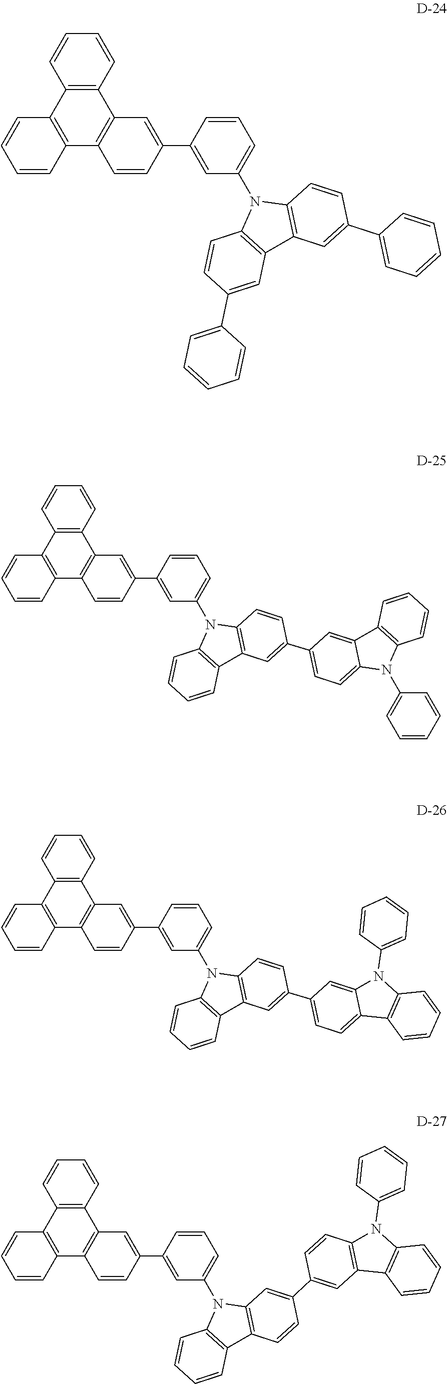 [SMILES: c1ccc(-c2ccc3c(c2)c2cc(-c4ccccc4)ccc2n3-c2cccc(-c3ccc4c5ccccc5c5ccccc5c4c3)c2)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1ccc(-n2c3ccccc3c3ccc(-c4ccc5c(c4)c4ccccc4n5-c4cccc(-c5ccc6c7ccccc7c7ccccc7c6c5)c4)cc32)cc1 ; c1ccc(-n2c3ccccc3c3cc(-c4ccc5c6ccccc6n(-c6cccc(-c7ccc8c9ccccc9c9ccccc9c8c7)c6)c5c4)ccc32)cc1]

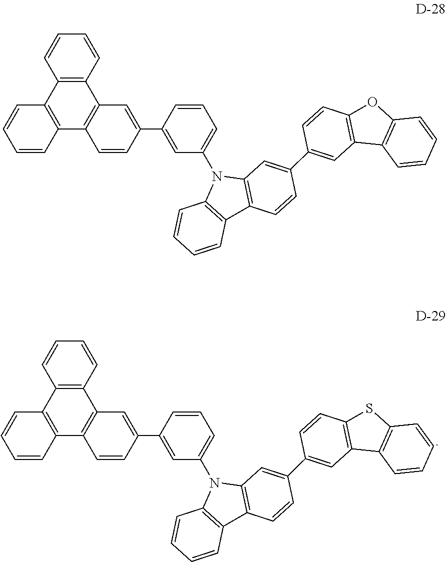 [SMILES: c1cc(-c2ccc3c4ccccc4c4ccccc4c3c2)cc(-n2c3ccccc3c3ccc(-c4ccc5oc6ccccc6c5c4)cc32)c1 ; c1cc(-c2ccc3c4ccccc4c4ccccc4c3c2)cc(-n2c3ccccc3c3ccc(-c4ccc5sc6ccccc6c5c4)cc32)c1]

20. The organic light-emitting device of claim 1, wherein
the emission layer comprises a dopant and a host,
the dopant comprises the organometallic compound, and
the host comprises at least one selected from the first to fourth compounds.
